(19) United States
(12) Patent Application Publication
(10) Pub. No.: US 2024/0409536 A1
(43) Pub. Date: Dec. 12, 2024

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Gulseher Sarah Sirin, Watertown, MA (US)

(21) Appl. No.: 18/688,088

(22) PCT Filed: Aug. 30, 2022
(86) PCT No.: PCT/US2022/075683

§ 371 Date: Feb. 29, 2024

Related U.S. Application Data
(60) Provisional application No. 63/238,424, filed on Aug. 30, 2021.
(60) Provisional application No. 63/238,687, filed on Aug. 30, 2021.
(60) Provisional application No. 63/238,693, filed on Aug. 30, 2021.
(60) Provisional application No. 63/282,902, filed on Nov. 24, 2021.
(60) Provisional application No. 63/393,202, filed on Jul. 28, 2022.

(51) Int. Cl. C07D 471/04; A61K 31/196; A61K 31/437; A61K 31/498; A61K 31/4985; A61K 31/502; C07C 233/92; C07D 237/28; C07D 241/44

(52) U.S. Cl. CPC C07D 471/04; A61K 31/196; A61K 31/437; A61K 31/498; A61K 31/4985; A61K 31/502; C07C 233/92; C07D 237/28; C07D 241/44

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/238,687, filed on Aug. 30, 2021; U.S. Application No. 63/238,693, filed on Aug. 30, 2021; U.S. Application No. 63/238,424, filed on Aug. 30, 2021; U.S. Application No. 63/282,902, filed on Nov. 24, 2021; and U.S. Application No. 63/393,202, filed on Jul. 28, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k)) and pharmaceutically acceptable salts, solvates, hydrates, tautomers, or stereoisomers thereof. The present disclosure additionally provides methods of using the compounds of the disclosure (e.g., compounds of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In another aspect, the present disclosure features a compound of Formula (I):

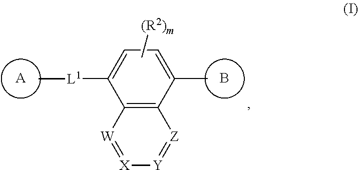

or a pharmaceutically acceptable salt, solvate, hydrate, tautomer, or stereoisomer thereof, wherein A, B, W, X, Y, Z, L¹, R², m, and subvariables thereof are as defined herein.

In another aspect, the present disclosure features a compound of Formula (II):

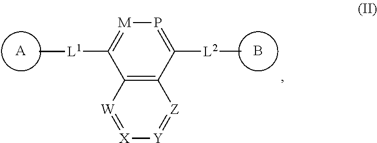

or a pharmaceutically acceptable salt, solvate, hydrate, tautomer, or stereoisomer thereof, wherein A, B, M, P, W, X, Y, Z, L¹, L², and subvariables thereof are as defined herein.

In another aspect, the present disclosure provides compounds of Formula (III):

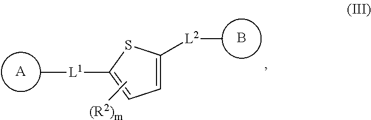

or a pharmaceutically acceptable salt, solvate, hydrate, tautomer, or stereoisomer thereof, wherein A, B, L¹, L², R², m, and subvariables thereof are as defined herein.

In another aspect, the present invention provides pharmaceutical compositions comprising a compound of Formula (I) (e.g., a compound of Formulas (I-a), (I-b), (I-c), (I-d), (I-e), (I-f), (I-g), (I-h), (I-i), (I-j), or (I-k)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) alters a target nucleic acid (e.g., DNA or RNA, e.g., a precursor RNA, e.g., a pre-mRNA), target protein, or combination thereof. In some embodiments, the compound of Formula (I) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue). In some embodiments, the presence of a compound of Formula (I) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue).

In another aspect, the present disclosure provides methods for preventing and/or treating a disease, disorder, or condition in a subject by administering a compound of Formula (I) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof in a biological sample or subject. In another aspect, the present disclosure provides methods of altering the isoform of a target protein with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k)) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I), (II), or (III) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k)) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I), (II), or (III) to a biological sample, a cell, or a subject comprises inhibition of cell growth or induction of cell death.

In another aspect, the present disclosure features kits comprising a container with a compound of Formula (I), (II), or (III) (e.g., a compound of Formulas (I-a), (I-b), (I-c), (I-d), (I-e), (I-f), (I-g), (I-h), (I-i), (I-j), (I-k), (I-k), (II-a), (II-b), (II-c), (II-d), (II-e), (III-a), (III-b), (III-c), (III-d), (III-e), (III-f), (III-g), (III-h), (III-i), (III-j), or (III-k), or a pharmaceutically acceptable salt, solvate, hydrate, tautomer, stereoisomer thereof, or a pharmaceutical composition thereof. In certain embodiments, the kits described herein further include instructions for administering the compound of Formula (I), (II), or (III) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, WO 2021/174164, WO 2021/174165, and WO 2021/174174. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972, WO 2021/174164, WO 2021/174165, and WO 2021/174174, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”).

In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substituent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I), (II), or (III) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), (II), or (III) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·×H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I), (II), or (III) refers to an amount sufficient to elicit the desired biological response, i.e., treating the condition. As will be appreciated by those of ordinary skill in this art, the effective amount of a compound of Formula (I), (II), or (III) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), (II), or (III) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I), (II), or (III)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I), (II), or (III)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure provides compounds of Formula (I-a):

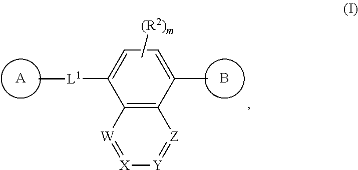

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, Y, and Z are each independently C(R^{3a}) or N, wherein at least one of W, X, Y, and Z is independently N; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; m is 0, 1, or 2; and x is 0, 1, or 2.

As generally described herein, A and B, are each independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

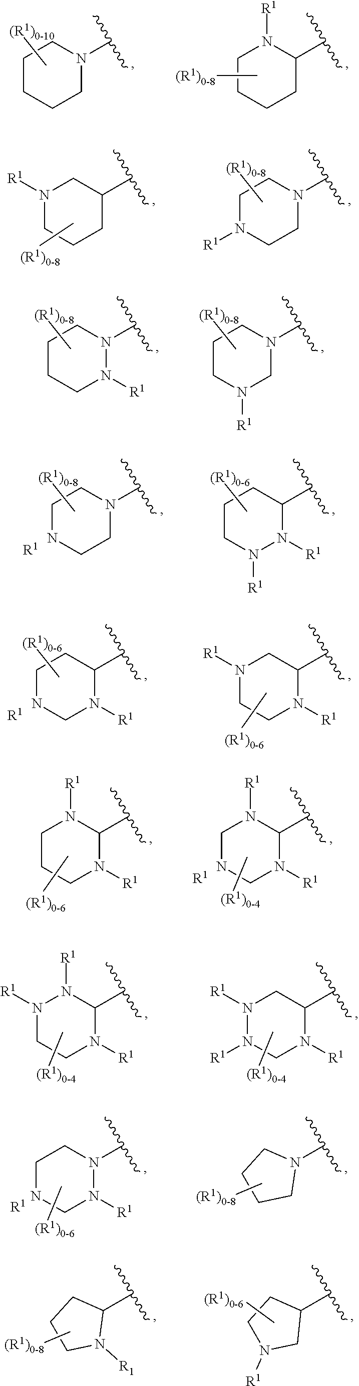

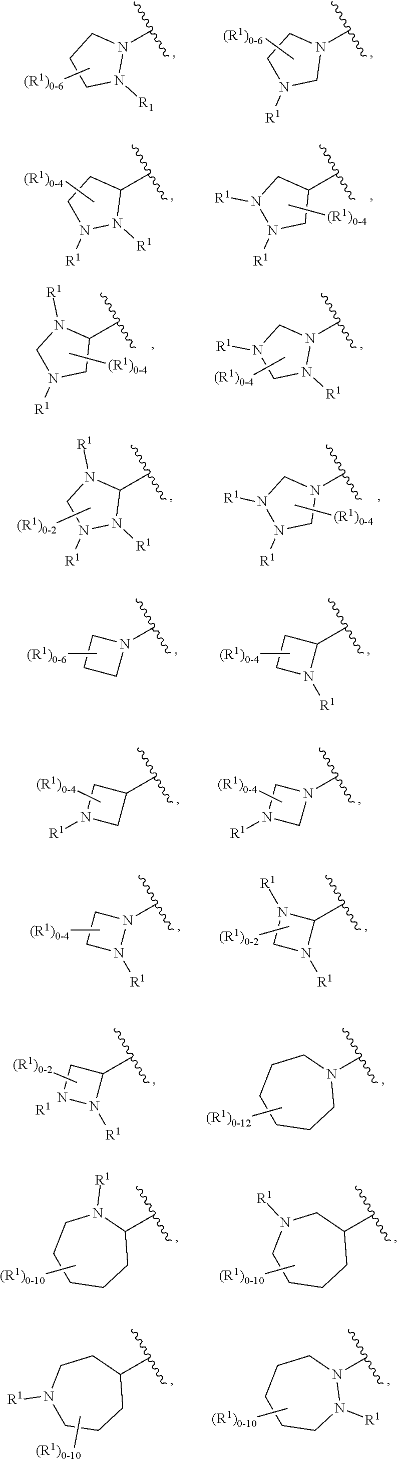

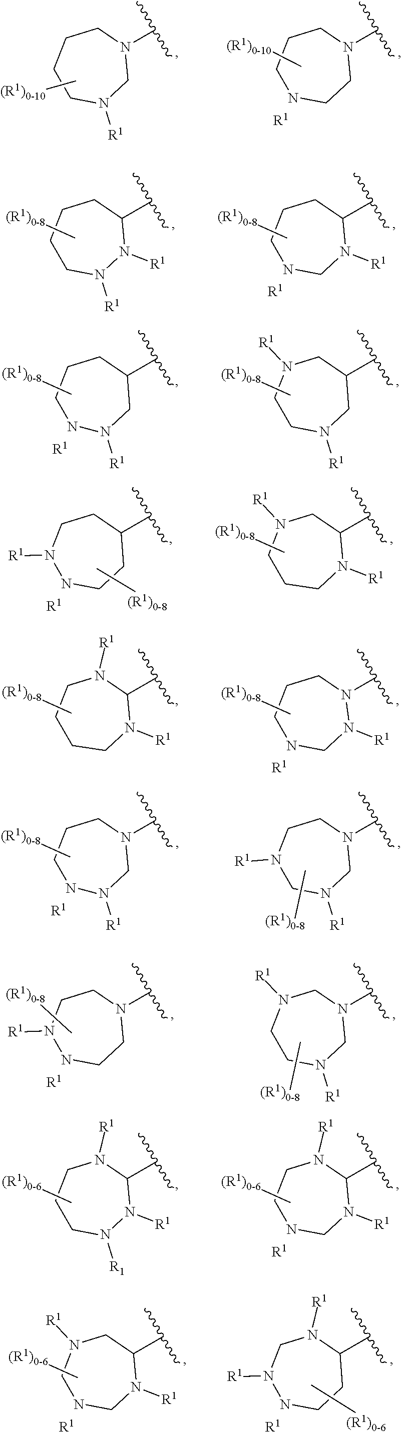

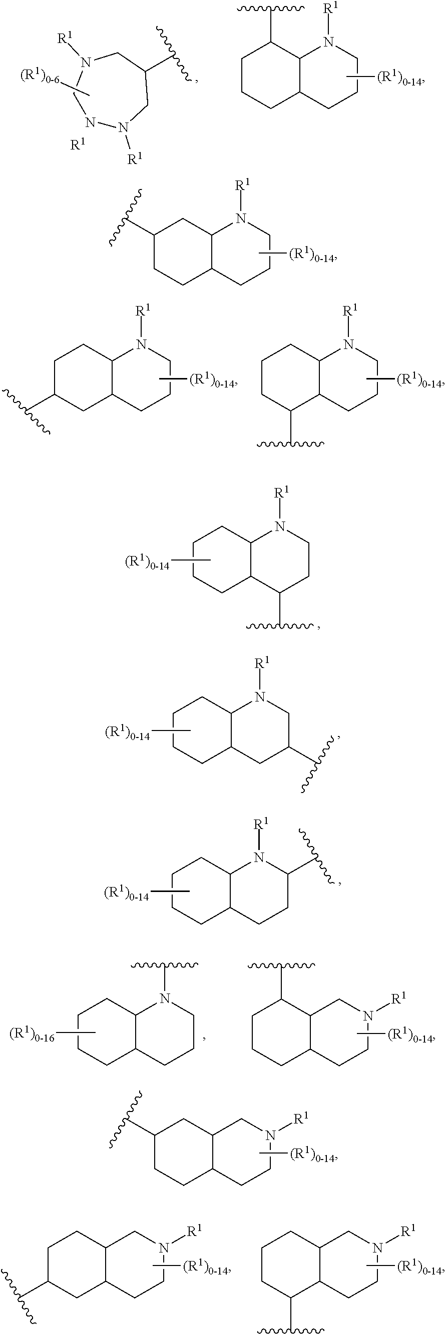

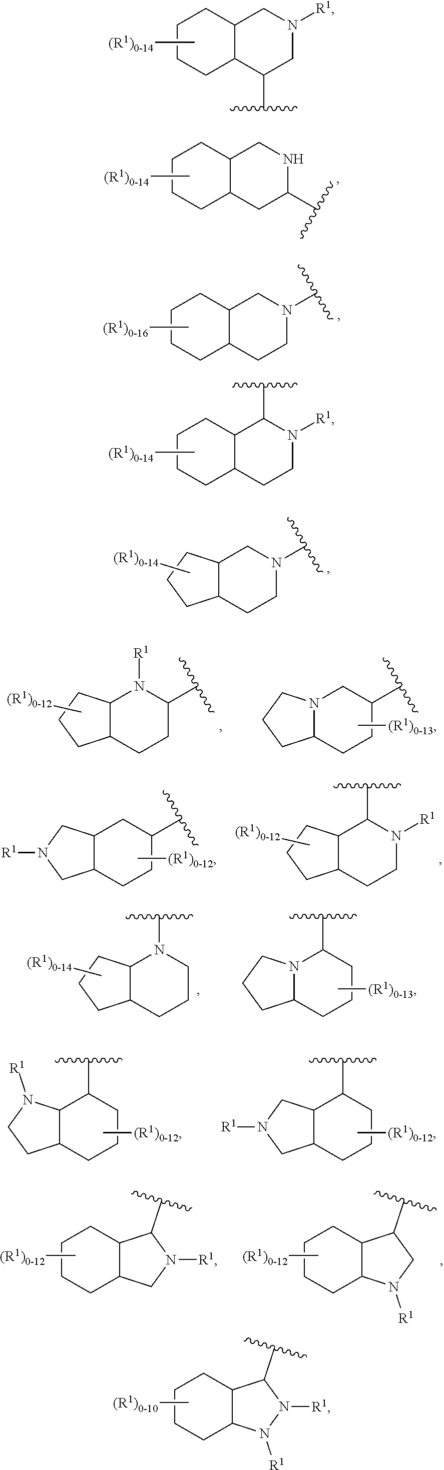

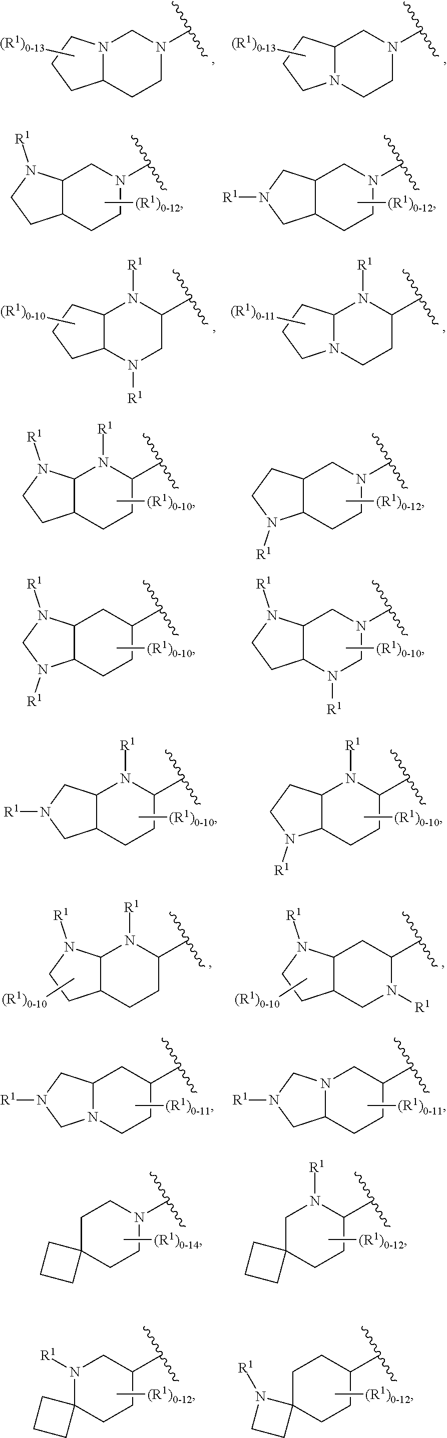

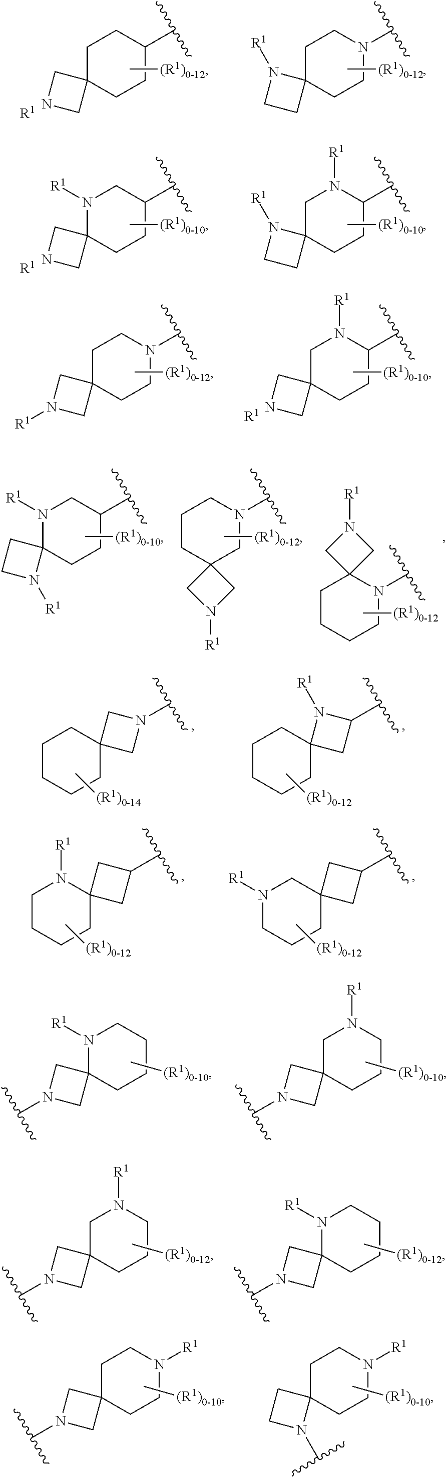

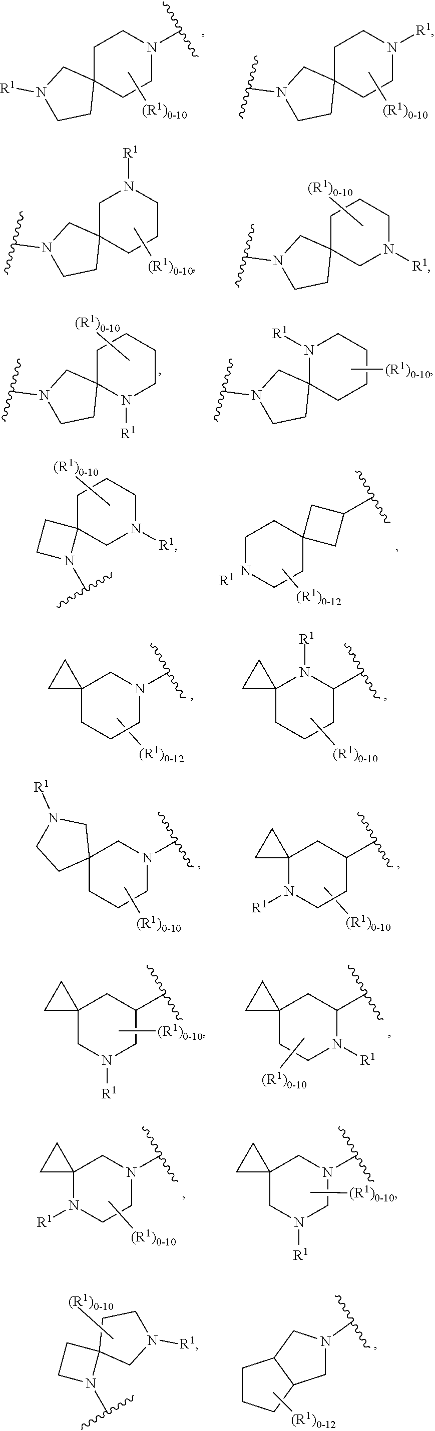

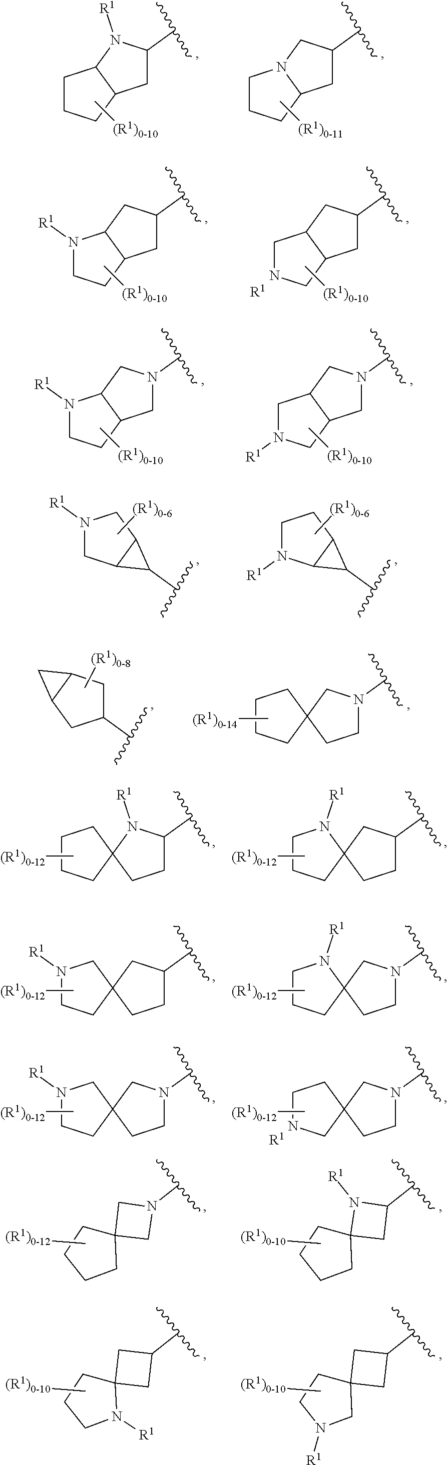

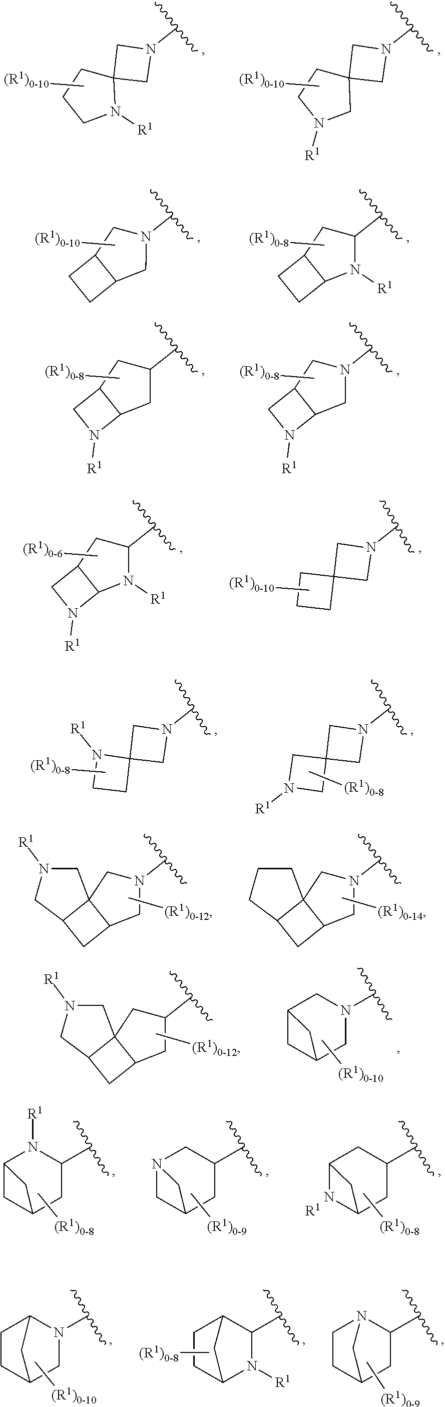

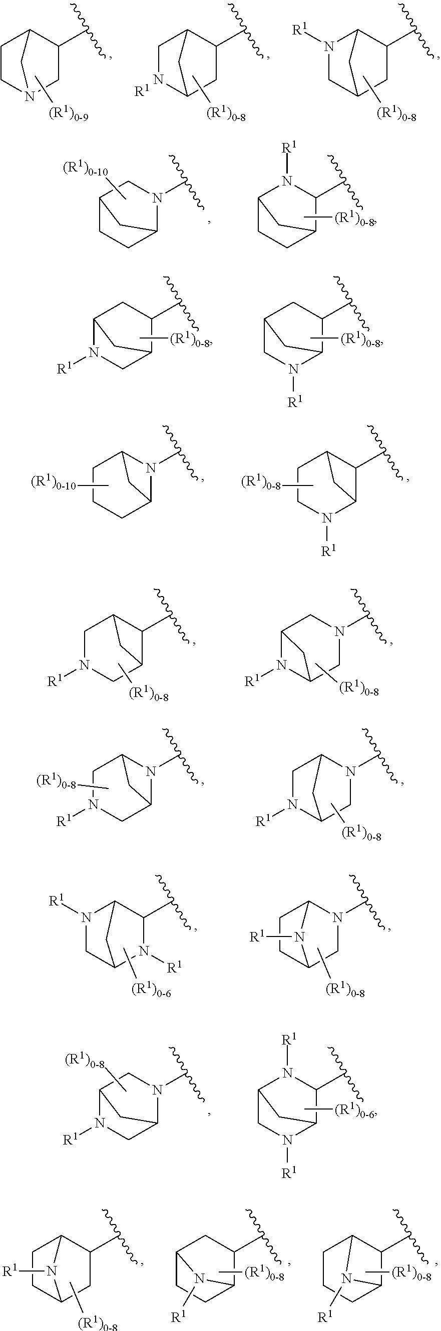

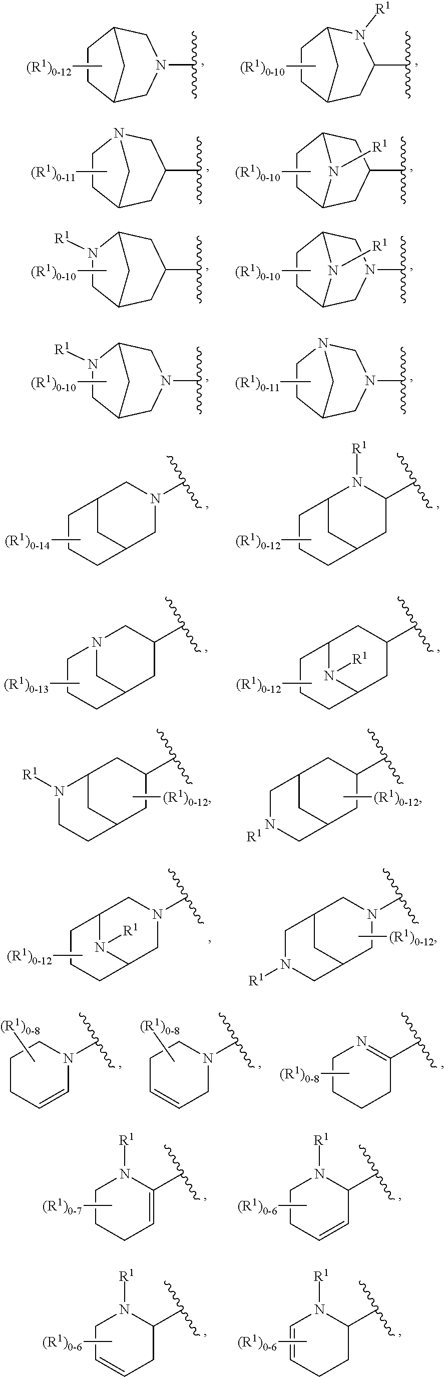

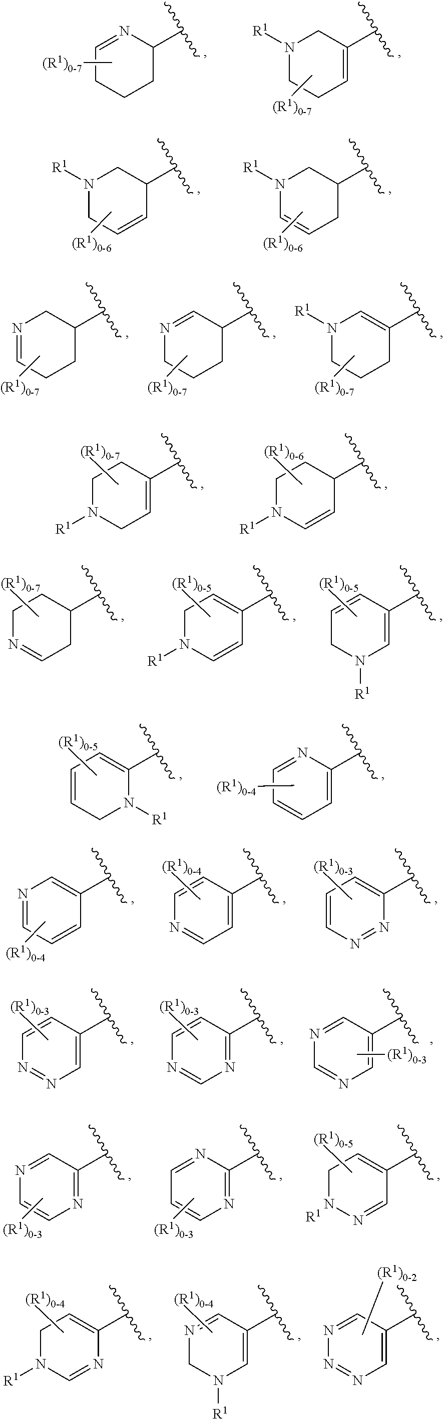

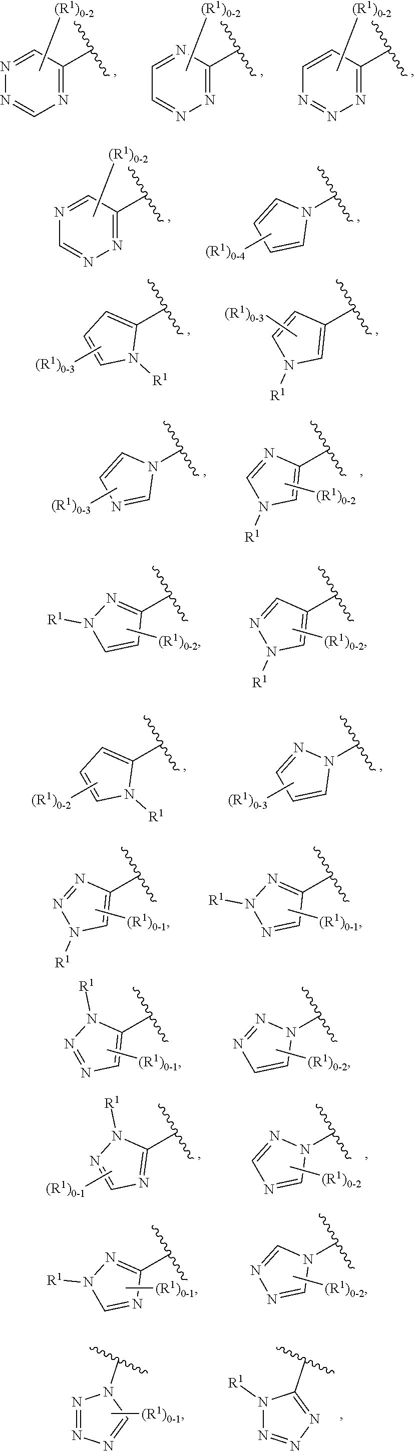

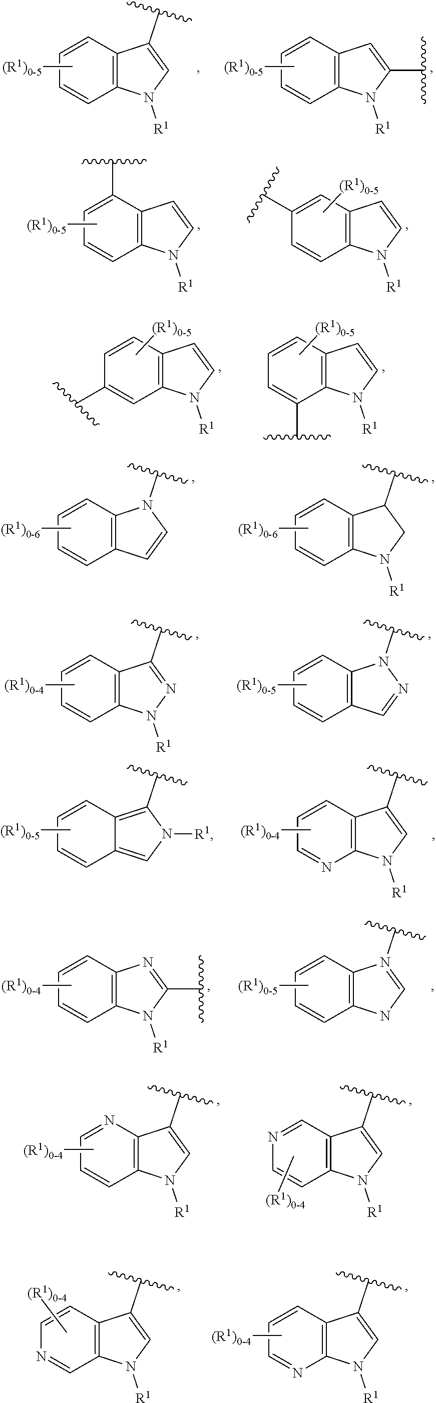

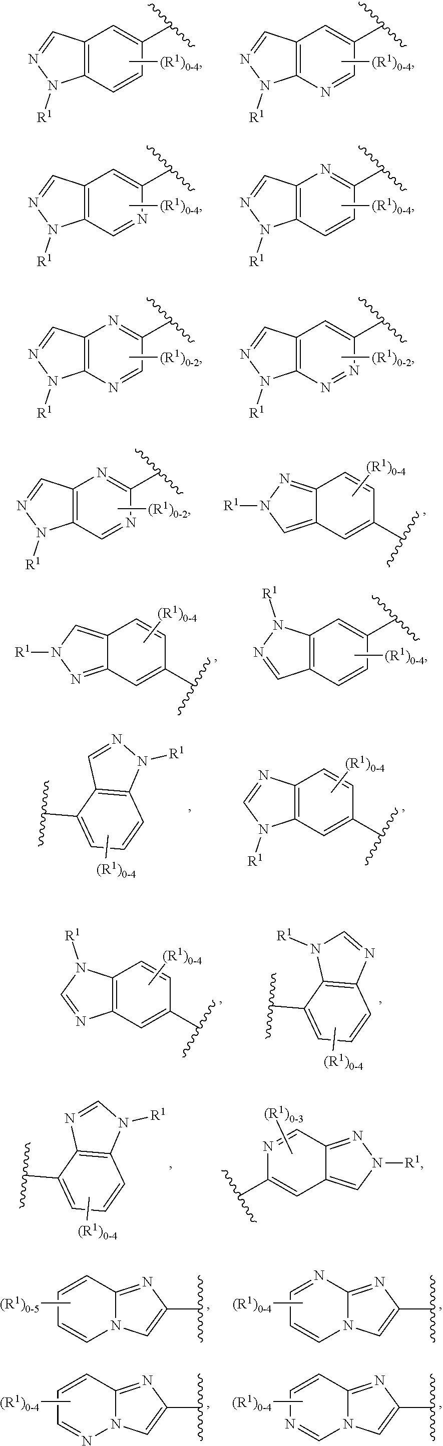

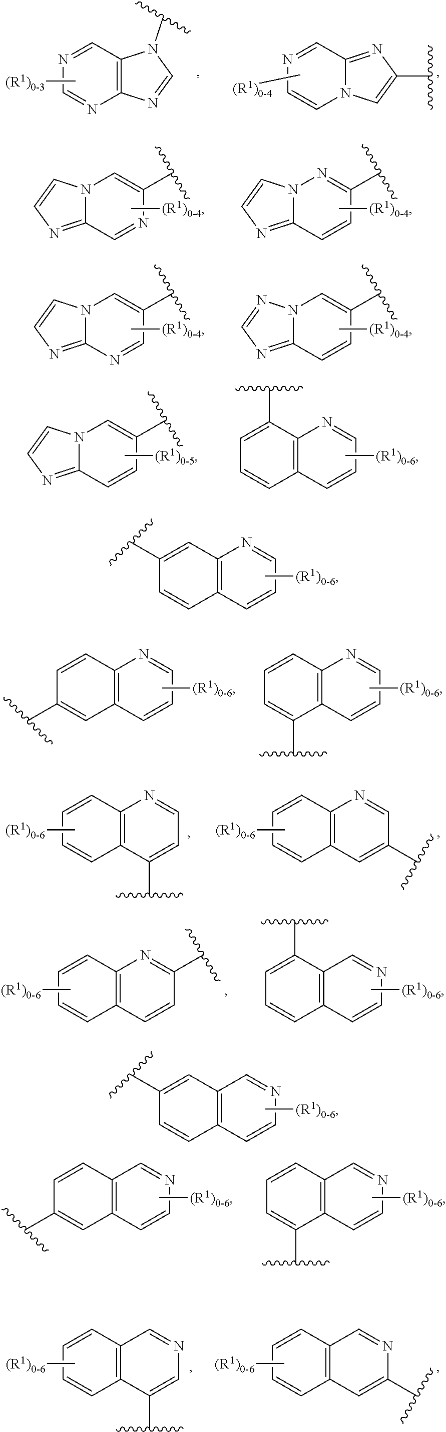

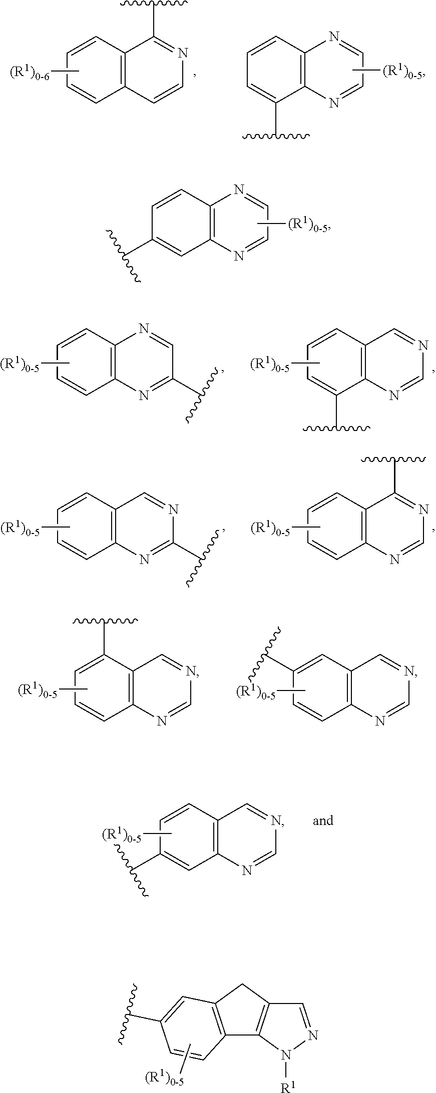

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

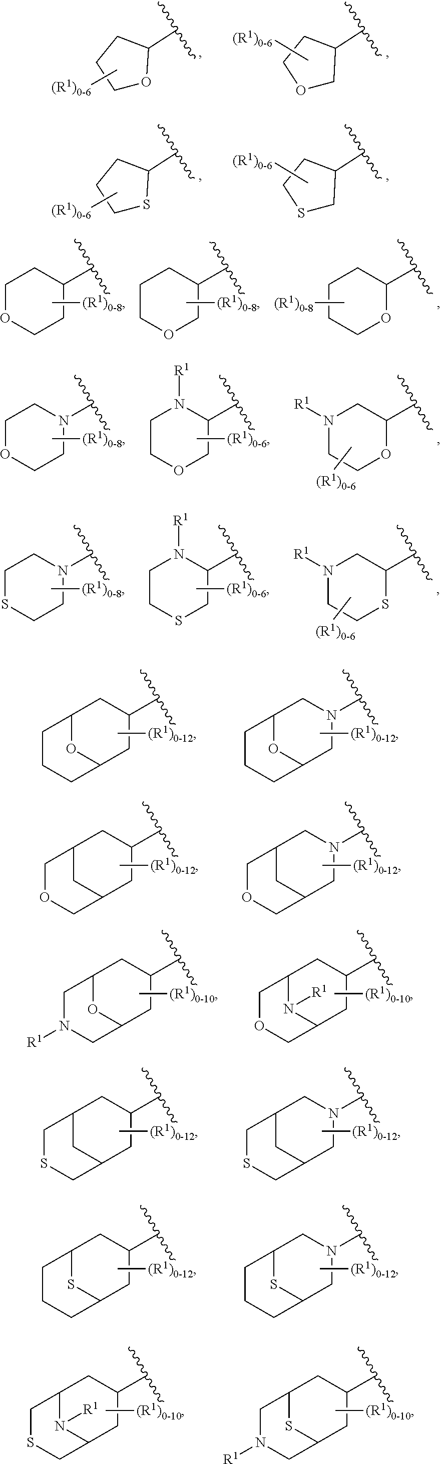

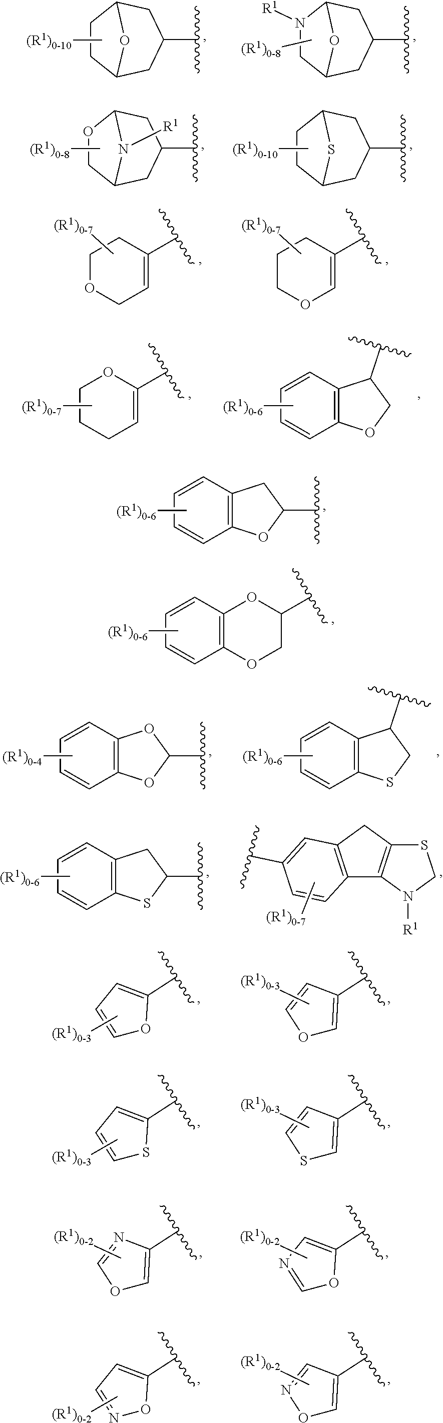

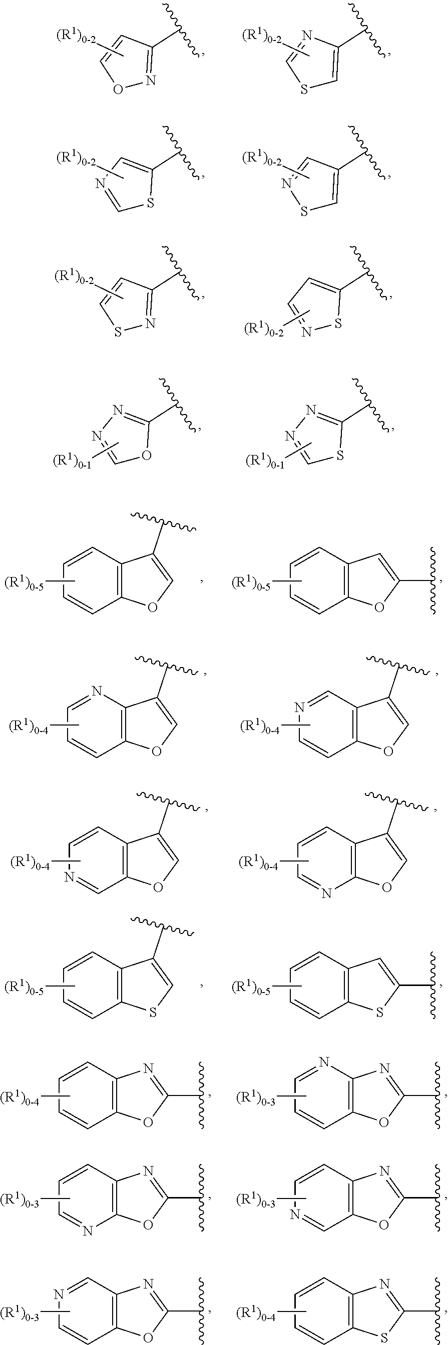

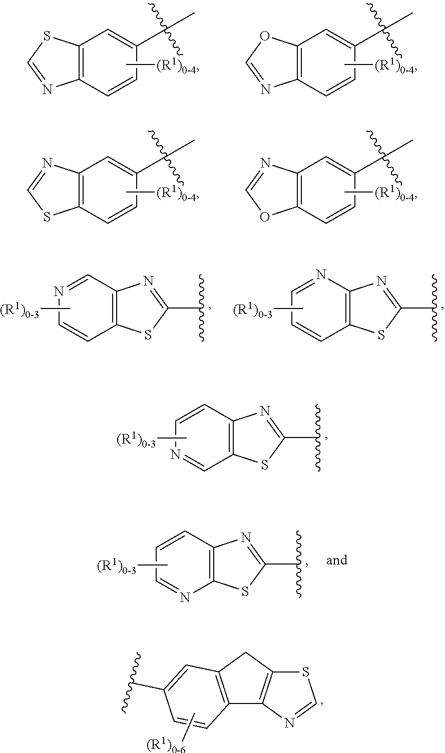

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, one of A and B is independently selected from

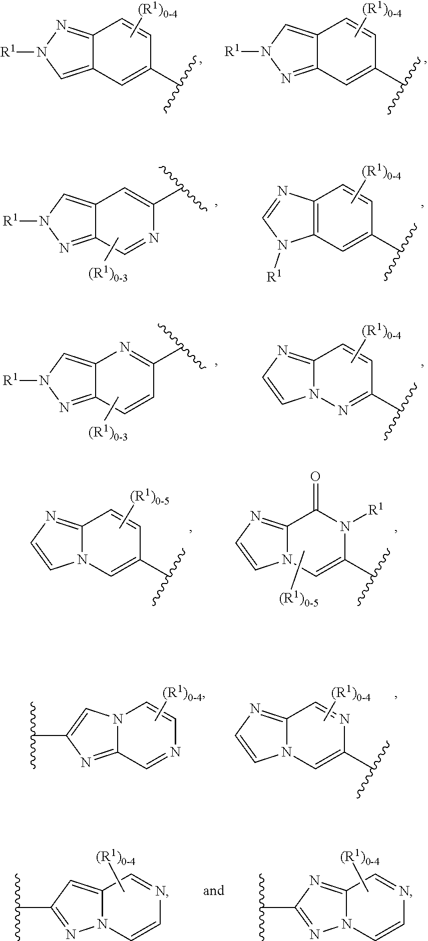

wherein R¹ is as described herein. In some embodiments, one of A and B is independently selected from

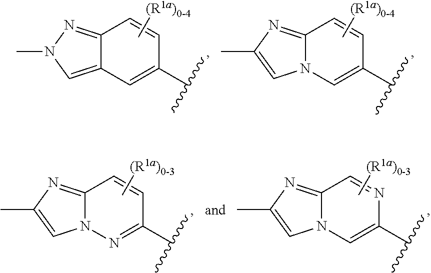

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, one of A and B is independently

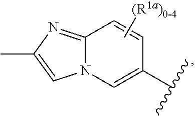

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, one of A and B is independently selected from

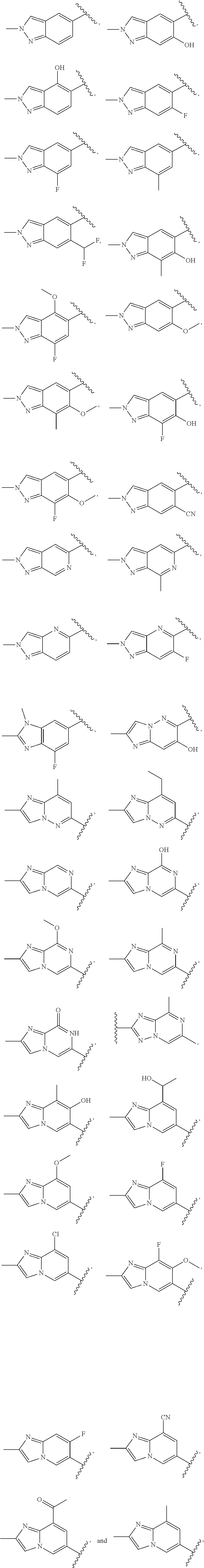

In some embodiments, one of A and B is independently selected from

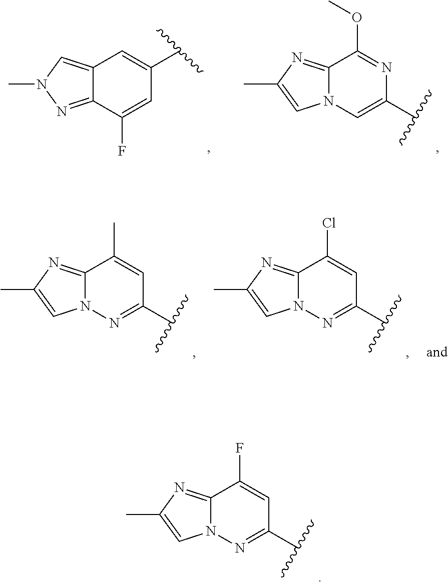 [SMILES: *c1cc(F)c2nn(C)cc2c1 ; *c1cn2cc(C)nc2c(OC)n1 ; *c1cc(C)c2nc(C)cn2n1 ; *c1cc(Cl)c2nc(C)cn2n1 ; *c1cc(F)c2nc(C)cn2n1]

In some embodiments, one of A and B is independently

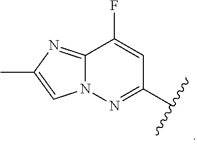 [SMILES: *c1cc(F)c2nc(C)cn2n1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-8 membered heterocyclyl optionally substituted with one or more R¹. In some embodiments, one of A and B is independently selected from

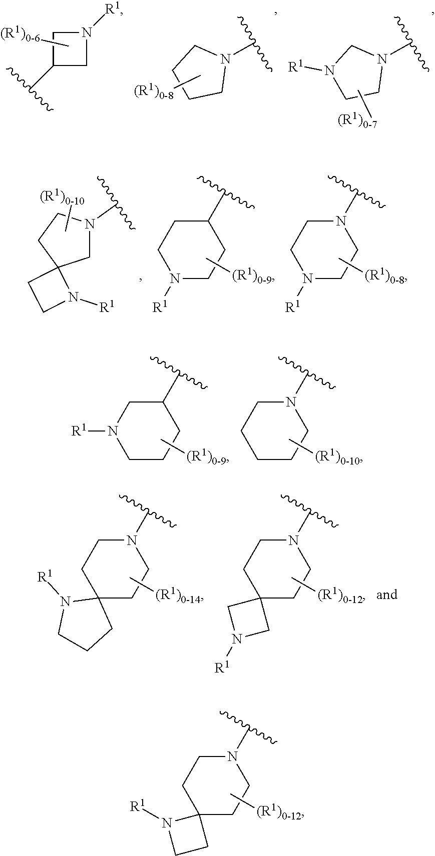

wherein R¹ is as described herein. In some embodiments, one of A and B is independently selected from

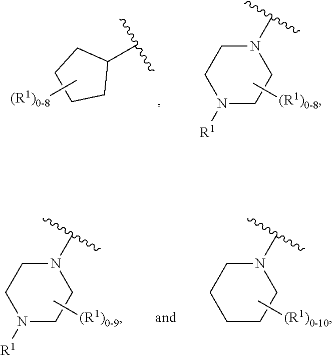

wherein R¹ is as described herein. In some embodiments, one of A and B is

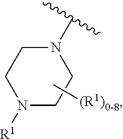

wherein R¹ is as described herein. In some embodiments, A is selected from

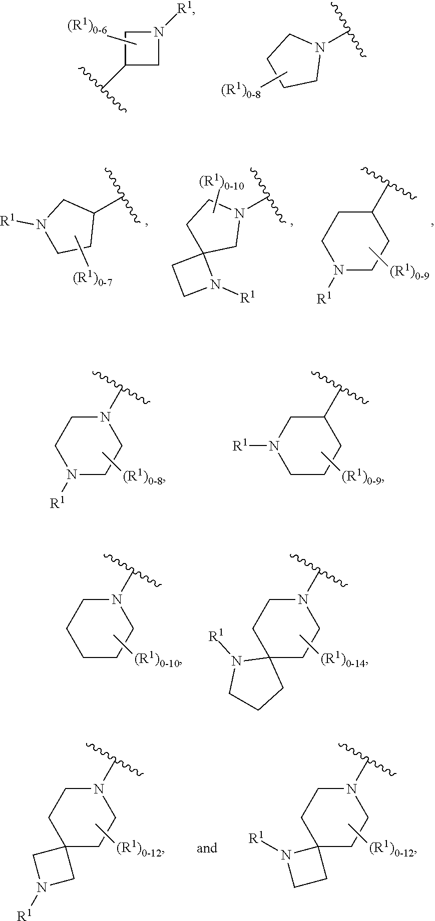

wherein R¹ is as described herein. In some embodiments, B is selected from

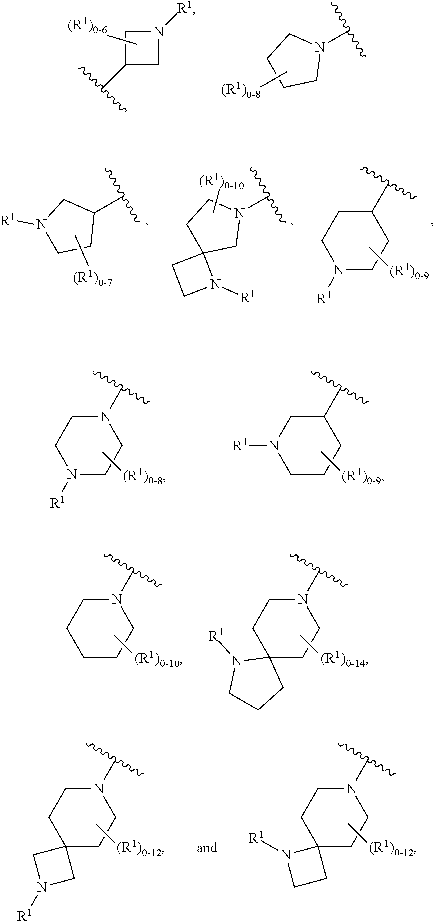

wherein R¹ is as described herein. In some embodiments, B is selected from

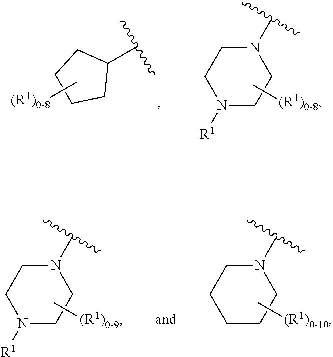

wherein R¹ is as described herein. In some embodiments, A is selected

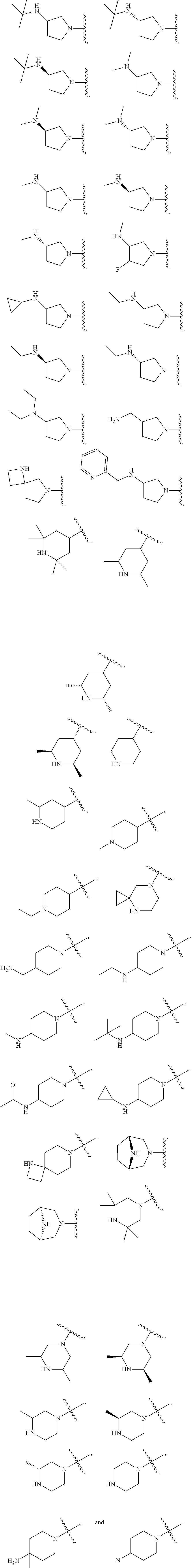

In some embodiments, A is selected one of A and B is independently selected from,

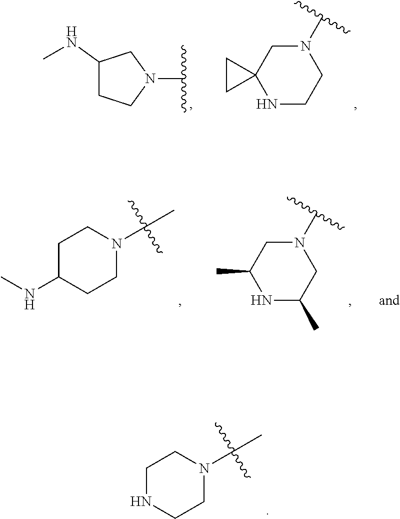

In some embodiments, one of A and B is

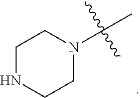 [SMILES: *N1CCNCC1]

As generally described herein, L¹ may be absent or refer to a C₁-C₆-alkylene, C₁-C₆-heteroalkylene, —O—, —C(O)—, —N(R⁴)—, —N(R⁴)C(O)—, or —C(O)N(R⁴)— group, wherein each alkylene and heteroalkylene is optionally substituted with one or more R⁵.

In some embodiments, L¹ is absent. In some embodiments, L¹ is C₁-C₆-alkylene (e.g., C₁-alkylene, C₂-alkylene, C₃-alkylene, C₄-alkylene, C₅-alkylene, or C₆-alkylene). In some embodiments, L¹ is unsubstituted C₁-C₆ alkylene. In some embodiments, L¹ is substituted C₁-C₆-alkylene, e.g., C₁-C₆ alkylene substituted with one or more R⁵. In some embodiments, L¹ is C₁-alkylene substituted with one R⁵. In some embodiments, L¹ is —CH₂—(or methylene). In some embodiments, L¹ is —C(O)—(or carbonyl).

As generally described herein, L¹ may be absent or refer to a C₁-C₆-alkylene, C₁-C₆-heteroalkylene, —O—, —C(O)—, —N(R⁴)—, —N(R⁴)C(O)—, or —C(O)N(R⁴)— group, wherein each alkylene and heteroalkylene is optionally substituted with one or more R⁵.

In some embodiments, L¹ is absent, C₁-C₆-alkylene, C₁-C₆-heteroalkylene, —N(R⁴)C(O)—, or —C(O)N(R⁴)—, wherein each alkylene and heteroalkylene is optionally substituted with one or more R⁵. In some embodiments, L¹ is unsubstituted C₁-C₆ heteroalkylene. In some embodiments, L¹ is substituted heteroalkylene, e.g., C₁-C₆ heteroalkylene substituted with one or more R⁵. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L¹ is —N(R⁴)C(O)—. In some embodiments, L¹ is —C(O)N(R⁴)—. In some embodiments, L¹ is —C(O)N(H)—.

In some embodiments, L¹ is nitrogen which is optionally substituted with R⁴. In some embodiments, L¹ is nitrogen substituted with R⁴. In some embodiments, L¹ is —N(R⁴)—, e.g., —N(CH₃)—. In some embodiments, L¹ is —NH—.

As generally described herein, W, X, Y, and Z each independently refer to C(R^{3a}) C(R^{3a})(R^{3b}), N, or N(R^{3c}), or O. In some embodiments, at least one of W, X, Y, and Z is either N or N(R^{3c}). In some embodiments, at least two of W, X, Y, and Z is N or N(R^{3c}). In some embodiments, at least two of X, Y, and Z is N or N(R^{3c}). In some embodiments, at least one of Y and Z is N or N(R^{3c}). In some embodiments, X is N. In some embodiments, X is N(R^{3c}) In some embodiments, at least one of W, X, Y, and Z is O. In some embodiments, X is O. In some embodiments, X is C(R^{3a}) (e.g., CH). In some embodiments, X is C(R^{3a})(R^{3b}). In some embodiments, Y is N. In some embodiments, Y is N(R^{3c}). In some embodiments, Y is C(R^{3a}) (e.g., CH). In some embodiments, Y is C(R^{3a})C(R^{3b}). In some embodiments, Z is N. In some embodiments, Z is N(R^{3c}). In some embodiments, Z is C(R^{3a}) (e.g., CH). In some embodiments, Z is C(R^{3a})C(R^{3b}). In some embodiments, two of X, Y, and Z are N, and the other of X, Y, and Z is C(R^{3a}) (e.g., CH). In some embodiments, one of X, Y, and Z is C(R^{3a}) (e.g., CH), and the others of X, Y, and Z are each independently N. In some embodiments, X and Y are each independently N, and Z is C(R^{3a}) (e.g., CH). In some embodiments, X is C(R^{3a}) (e.g., CH), and Y and Z are each independently N.

In some embodiments, W is C(R^{3a}) (e.g., CH) or C(R^{3a})(R^{3b}) (e.g., CH₂). In some embodiments, W is C(R^{3a}) (e.g., CH). In some embodiments, W is C(R^{3a})(R^{3b}). In some embodiments, W is C(R^{3a}) (e.g., CH), two of X, Y, and Z are N, and the other of X, Y, and Z is C(R^{3a}) (e.g., CH). In some embodiments, W is C(R^{3a}) (e.g., CH), one of X, Y, and Z is C(R^{3a}) (e.g., CH), and the others of X, Y, and Z are each independently N. In some embodiments, X and Y are each independently N, and W and Z are each independently C(R^{3a}) (e.g., CH). In some embodiments, W and X are each independently C(R^{3a}) (e.g., CH), and Y and Z are each independently N.

In some embodiments, X, Y, and Z are each independently N or C(R^{3a}), wherein at least one of X, Y, and Z is N and the bonds in the ring comprising X, Y, and Z may be single or double bonds as valency permits.

In some embodiments, X is C(R^{3a}), Y is C(R^{3a}), and Z is O. In some embodiments, X is C(R^{3a}), Y is C(R^{3a}), Z is O, and y is 0. In some embodiments, X is C(R^{3a}), Y is C(R^{3a}), Z is O, and the bond between X and Y is a double bond. In some embodiments, X is C(R^{3a}), Y is C(R^{3a}), Z is O, and the bond between Y and Z is a single bond.

In some embodiments,

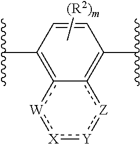

is selected from

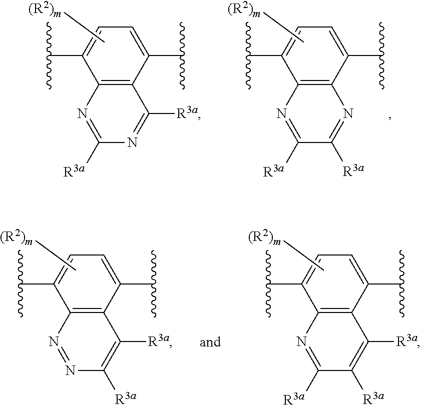

wherein R^{3a} is as defined herein. In some embodiments,

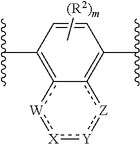

is selected from

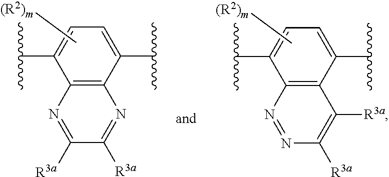

wherein R^{3a} is as defined herein. In some embodiments, R^{3a} is C₁-C₆-alkyl or C₁-C₆-heteroalkyl.

In some embodiments,

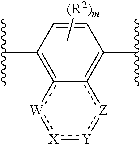

is selected from

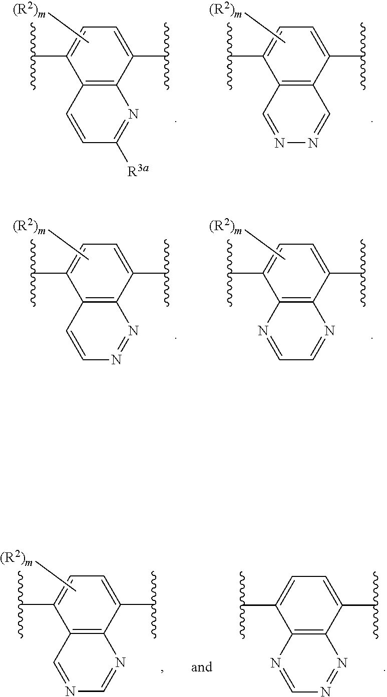

In some embodiments,

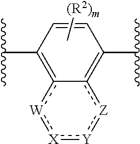

is selected from

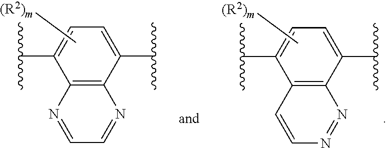

In some embodiments,

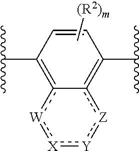

is

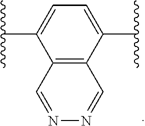 [SMILES: *c1ccc(*)c2cnncc12]

In some embodiments

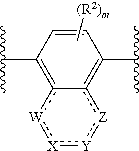

is

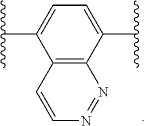 [SMILES: *c1ccc(*)c2nnccc12]

In some embodiments,

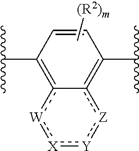

is

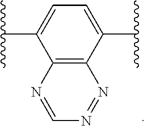 [SMILES: *c1ccc(*)c2nncnc12]

In some embodiments

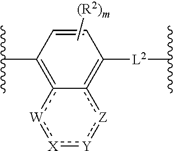

is selected from

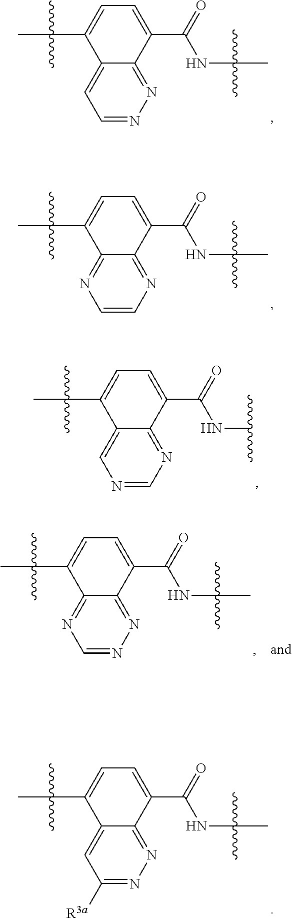

In some embodiments,

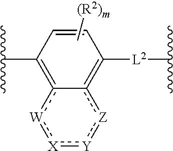

is

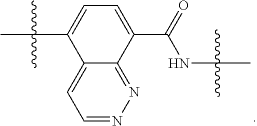 [SMILES: *NC(=O)c1ccc(*)c2ccnnc12]

In some embodiments,

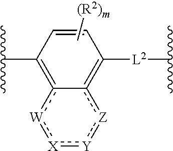

is

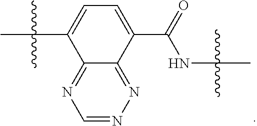 [SMILES: *NC(=O)c1ccc(*)c2ncnnc12]

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁶. In some embodiments, R¹ is C₂-C₆-alkenyl substituted with one or more R⁶. In some embodiments, R¹ is C₂-C₆-alkynyl substituted with one or more R⁶. In some embodiments, R¹ is C₁-C₆-heteroalkyl substituted with one or more R⁶. In some embodiments, R¹ is C₁-C₆-haloalkyl substituted with one or more R⁶. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁶. In some embodiments, R¹ is heterocyclyl substituted with one or more R⁶. In some embodiments, R¹ is aryl substituted with one or more R⁶. In some embodiments, R¹ is C₁-C₆ alkylene-aryl substituted with one or more R⁶. In some embodiments, R¹ is C₁-C₆ alkenylene-aryl substituted with one or more R⁶. In some embodiments, R¹ is C₁-C₆ alkylene-heteroaryl substituted with one or more R⁶. In some embodiments, R¹ is heteroaryl substituted with one or more R⁶.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is —C(O)NR^{B}R^{C}. In some embodiments, R¹ is —C(O)R^{D}. In some embodiments, R¹ is —C(O)OR^{D}. In some embodiments, R¹ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁶.

In some embodiments, R² is hydrogen. In some embodiments, R² is halo (e.g., fluoro, chloro, bromo, or iodo). In some embodiments, R² is cyano. In some embodiments, R² is C₁-C₆-alkyl. In some embodiments, R² is C₂-C₆-alkenyl. In some embodiments, R² is C₂-C₆-alkynyl. In some embodiments, R² is —OR^{A} (e.g., —OH).

In some embodiments, R^{3a} is hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}. In some embodiments, R^{3a} is hydrogen or C₁-C₆-alkyl. In some embodiments, R^{3a} is hydrogen. In some embodiments, R^{3a} is C₁-C₆-alkyl (e.g., methyl). In some embodiments, R^{3a} is halo (e.g., fluoro, chloro, bromo, or iodo). In some embodiments, R^{3a} is cyano. In some embodiments, R^{3a} is —OR^{A} (e.g., —OH). In some embodiments, R^{3a} is —NR^{B}R^{C}. In some embodiments, R^{3a} is —C(O)R^{D}. In some embodiments, R^{3a} is —C(O)OR^{D}.

In some embodiments, R⁴ is hydrogen. In some embodiments, R⁴ is C₁-C₆ alkyl. In some embodiments, R⁴ is C₁-C₆ haloalkyl (e.g., —CF₃ or —CHF₂). In some embodiments, R⁴ is methyl.

In some embodiments, R⁵ is hydrogen. In some embodiments, R⁵ is C₁-C₆-alkyl. In some embodiments, R⁵ is C₁-C₆-heteroalkyl. In some embodiments, R⁵ is C₁-C₆-haloalkyl. In some embodiments, R⁵ is cycloalkyl. In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}. In some embodiments, R⁵ is —NR^{B}R^{C}. In some embodiments, R⁵ is —C(O)R^{D} or —C(O)OR^{D}.

In some embodiments, R⁶ is C₁-C₆-alkyl. In some embodiments, R⁶ is C₂-C₆-alkenyl. In some embodiments, R⁶ is C₂-C₆-alkynyl. In some embodiments, R⁶ is C₁-C₆-heteroalkyl. In some embodiments, R⁶ is C₁-C₆-haloalkyl. In some embodiments, R⁶ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁶ is C₁-C₆-alkyl substituted with one or more R¹¹. In some embodiments, R⁶ is C₂-C₆-alkenyl substituted with one or more R¹¹. In some embodiments, R⁶ is C₂-C₆-alkynyl substituted with one or more R¹¹. In some embodiments, R⁶ is C₁-C₆-haloalkyl substituted with one or more R¹¹. In some embodiments, R⁶ is C₁-C₆-heteroalkyl substituted with one or more R¹¹.

In some embodiments, R⁶ is cycloalkyl. In some embodiments, R⁶ is heterocyclyl. In some embodiments, R⁶ is aryl. In some embodiments, R⁶ is heteroaryl. In some embodiments, R⁶ is unsubstituted cycloalkyl, unsubstituted heterocyclyl, unsubstituted aryl, or unsubstituted heteroaryl. In some embodiments, R⁶ is cycloalkyl substituted with one or more R¹¹. In some embodiments, R⁶ is heterocyclyl substituted with one or more R¹¹. In some embodiments, R⁶ is aryl substituted with one or more R¹¹. In some embodiments, R⁶ is heteroaryl substituted with one or more R¹¹.

In some embodiments, R⁶ is halo (e.g., fluoro, chloro, bromo, or iodo). In some embodiments, R⁶ is cyano. In some embodiments, R⁶ is oxo. In some embodiments, R⁶ is —OR^{A}. In some embodiments, R⁶ is —NR^{B}R^{C}. In some embodiments, R⁶ is —NR^{B}C(O)R^{D}. In some embodiments, R⁶ is —NO₂. In some embodiments, R⁶ is —C(O)NR^{B}R^{C}. In some embodiments, R⁶ is —C(O)R^{D}. In some embodiments, R⁶ is —C(O)OR^{D}. In some embodiments, R⁶ is —SR^{E}. In some embodiments, R⁶ is —S(O)ₓR^{D}.

In some embodiments, R⁷ is C₁-C₆-alkyl. In some embodiments, R⁷ is halo (e.g., fluoro, chloro, bromo, or iodo). In some embodiments, R⁷ is cyano. In some embodiments, R⁷ is oxo. In some embodiments, R⁷ is —OR^{A1} (e.g., —OH).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl.

In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁷.

In some embodiments, R^{D}, R^{E}, or both are independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, each of R^{D} and R^{E} is independently C₁-C₆ alkyl. In some embodiments, R^{D} is hydrogen. In some embodiments, R^{E} is hydrogen. In some embodiments, R^{D} is C₁-C₆ alkyl (e.g., methyl). In some embodiments, R^{E} is C₁-C₆ alkyl (e.g., methyl). In some embodiments, R^{D} is C₁-C₆ heteroalkyl. In some embodiments, R^{E} is C₁-C₆ heteroalkyl. In some embodiments, R^{D} is C₁-C₆ haloalkyl. In some embodiments, R^{E} is C₁-C₆ haloalkyl. In some embodiments, R^{D} is cycloalkyl. In some embodiments, R^{E} is cycloalkyl. In some embodiments, R^{D} is heterocyclyl. In some embodiments, R^{E} is heterocyclyl. In some embodiments, R^{D} is aryl. In some embodiments, R^{E} is aryl. In some embodiments, R^{D} is heteroaryl. In some embodiments, R^{E} is heteroaryl. In some embodiments, R^{D} is C₁-C₆ alkylene-aryl (e.g., benzyl). In some embodiments, R^{E} is C₁-C₆ alkylene-aryl (e.g., benzyl). In some embodiments, R^{D} is C₁-C₆ alkylene-heteroaryl. In some embodiments, R^{E} is C₁-C₆ alkylene-heteroaryl.

In some embodiments, R^{A1} is hydrogen. In some embodiments, R^{A1} is C₁-C₆-alkyl (e.g., methyl).

In some embodiments, m is 0, 1, or 2. In some embodiments, m is 0. In some embodiments, m is 1. In some embodiments, m is 2. In some embodiments, x is 0, 1, or 2. In some embodiments, x is 0. In some embodiments, x is 1. In some embodiments, x is 2. In some embodiments y is 0 or 1. In some embodiments, y is 0. In some embodiments, y is 1.

In some embodiments, the compound is a compound of Formula (I):

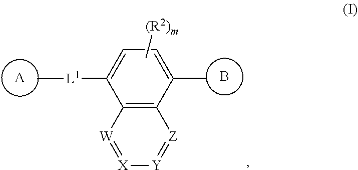

or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
W, X, Y, and Z are each independently C(R^{3a}) or N, wherein at least one of W, X, Y, and Z is independently N;
L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶
each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A};
R^{3a} is hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D};
each R⁴ is independently hydrogen, C₁-C₆-alkyl, or C₁-C₆-haloalkyl;
each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1};
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is hydrogen or C₁-C₆-alkyl;
m is 0, 1, or 2; and
x is 0, 1, or 2

In some embodiments the compound is a compound of Formula (I-a):

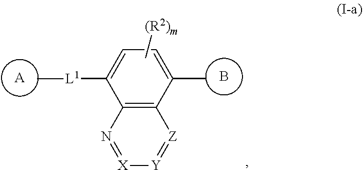

or a pharmaceutically acceptable salt, solvate, hydrate, tautomer, or stereoisomer thereof, wherein: A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), or O, wherein at least one of X, Y, and Z is N, N(R^{3c}), or O, and the bonds in the ring comprising X, Y, and Z may be single or double bonds as valency permits; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen or C₁-C₆-alkyl; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound is a compound of Formula (I-b):

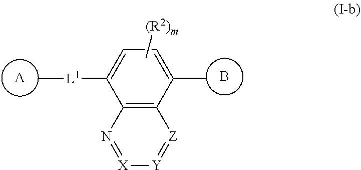

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}) or N; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-c):

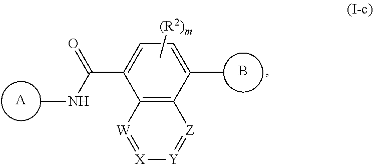

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, Y, and Z are each independently C(R^{3a}) or N, wherein at least one of W, X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-d):

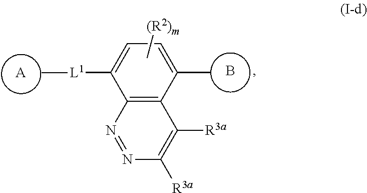

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-e):

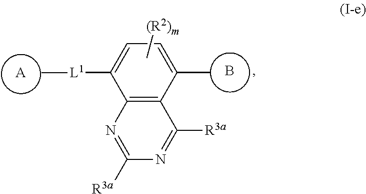

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-f):

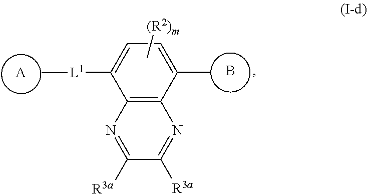

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-g):

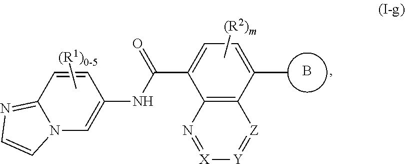

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; X, Y, and Z are each independently C(R^{3a}) or N, wherein at least one of X, Y, and Z is independently N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-h):

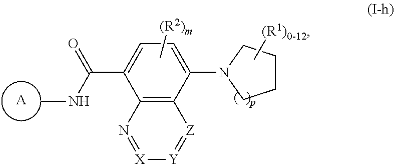

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; X, Y, and Z are each independently C(R^{3a}) or N, wherein at least one of X, Y, and Z is independently N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E} or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; p is 1, 2, 3, or 4; and x is 0, 1, or 2.

In some embodiments, the compound of Formula (I) is a compound of Formula (I-i):

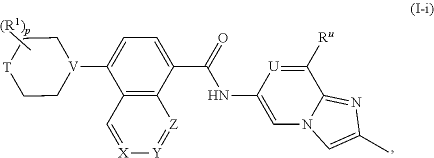

or a pharmaceutically acceptable salt, solvate, hydrate, tautomer, or stereoisomer thereof, wherein: X, Y, and Z are each independently C(R^{3a}) or N, wherein at least one of X, Y, and Z is N; T is N(R¹) or C(R¹)₂; U and V are each independently N or C; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl are optionally substituted with one or more R⁶; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; R^{U} is halo, C₁-C₆-alkyl, or C₁-C₆-haloalkyl; and p is 0, 1, or 2.

In some embodiments, the compound of Formula (I) is a compound of Formula (I-j):

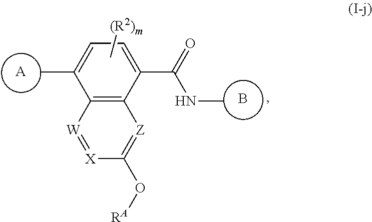

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, and Z are each independently C(R^{3a}) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}, wherein each alkyl, haloalkyl, aryl, heteroaryl, alkylene-aryl, alkylene-heteroaryl is optionally substituted with one or more R¹¹; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; x is 0, 1, or 2; and y is 1.

In some embodiments the compound of Formula (I) is a compound of Formula (I-k):

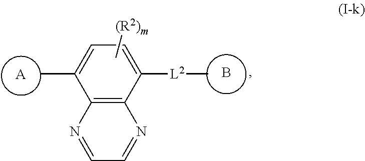

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl are optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-1):

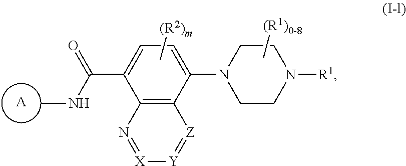

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; X, Y, and Z are each independently C(R^{3a}) or N, wherein at least one of X, Y, and Z is independently N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, NR^{B}R^{C}, —C(O)R^{D}, or —C(O)OR^{D}; each R^{3a} is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E} or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Compound | |
|---|---|
| No. | Structure |
| 100 | 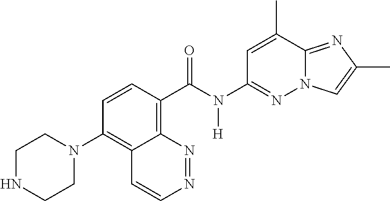 [SMILES: Cc1cn2nc(NC(=O)c3ccc(N4CCNCC4)c4ccnnc34)cc(C)c2n1] |
| 101 | 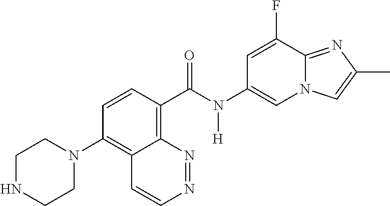 [SMILES: Cc1cn2cc(NC(=O)c3ccc(N4CCNCC4)c4ccnnc34)cc(F)c2n1] |
| 103 | 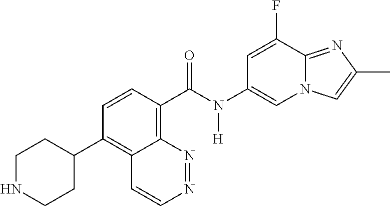 [SMILES: Cc1cn2cc(NC(=O)c3ccc(C4CCNCC4)c4ccnnc34)cc(F)c2n1] |
| 104 | 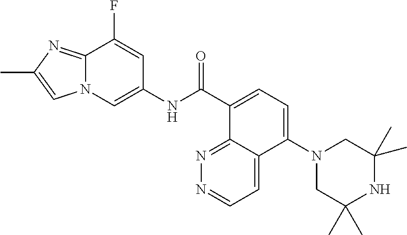 |
| 105 | 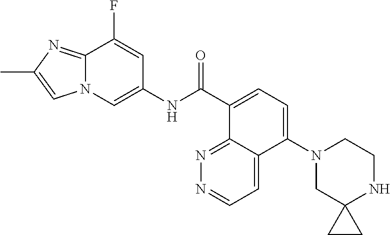 [SMILES: Cc1cn2cc(NC(=O)c3ccc(N4CCNC5(CC5)C4)c4ccnnc34)cc(F)c2n1] |
| 106 | 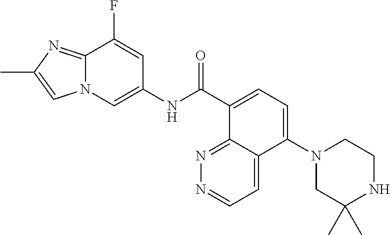 [SMILES: Cc1cn2cc(NC(=O)c3ccc(N4CCNC(C)(C)C4)c4ccnnc34)cc(F)c2n1] |
| 107 | 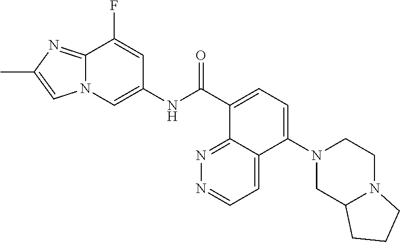 [SMILES: Cc1cn2cc(NC(=O)c3ccc(N4CCN5CCCC5C4)c4ccnnc34)cc(F)c2n1] |
| 108 | 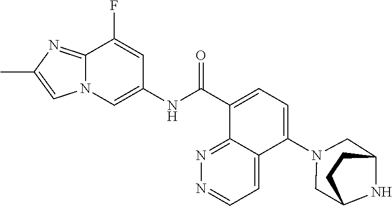 |
| 109 | 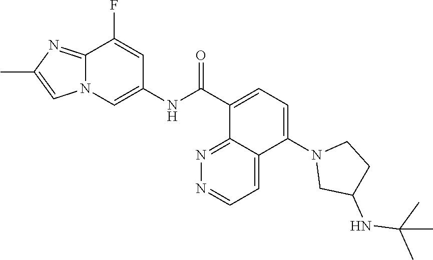 [SMILES: Cc1cn2cc(NC(=O)c3ccc(N4CCC(NC(C)(C)C)C4)c4ccnnc34)cc(F)c2n1] |
| 110 | 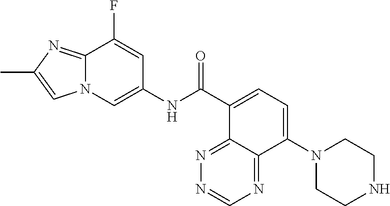 [SMILES: Cc1cn2cc(NC(=O)c3ccc(N4CCNCC4)c4ncnnc34)cc(F)c2n1] |
| 111 | 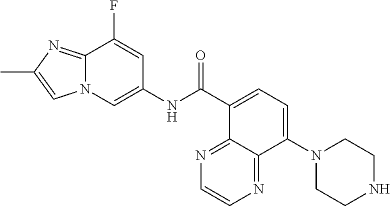 [SMILES: Cc1cn2cc(NC(=O)c3ccc(N4CCNCC4)c4nccnc34)cc(F)c2n1] |
| 112 | 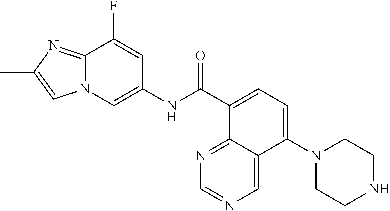 [SMILES: Cc1cn2cc(NC(=O)c3ccc(N4CCNCC4)c4cncnc34)cc(F)c2n1] |
| 113 | 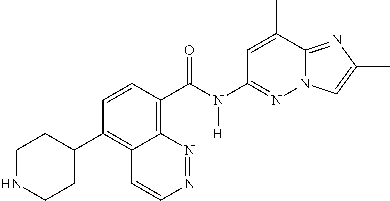 [SMILES: Cc1cn2nc(NC(=O)c3ccc(C4CCNCC4)c4ccnnc34)cc(C)c2n1] |
| 114 | 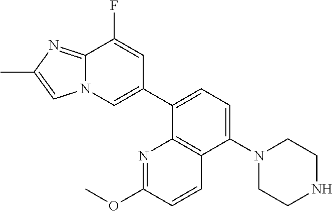 [SMILES: COc1ccc2c(N3CCNCC3)ccc(-c3cc(F)c4nc(C)cn4c3)c2n1] |
| 115 | 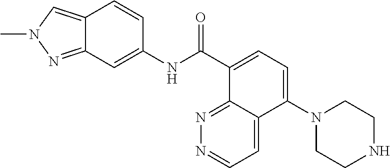 [SMILES: Cn1cc2ccc(NC(=O)c3ccc(N4CCNCC4)c4ccnnc34)cc2n1] |
| 116 | 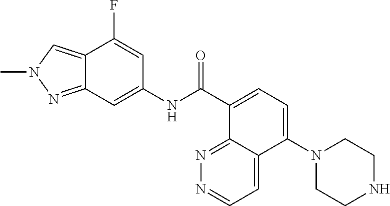 [SMILES: Cn1cc2c(F)cc(NC(=O)c3ccc(N4CCNCC4)c4ccnnc34)cc2n1] |
| 117 | 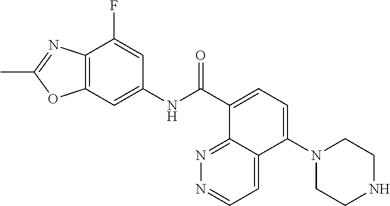 [SMILES: Cc1nc2c(F)cc(NC(=O)c3ccc(N4CCNCC4)c4ccnnc34)cc2o1] |
| 118 | 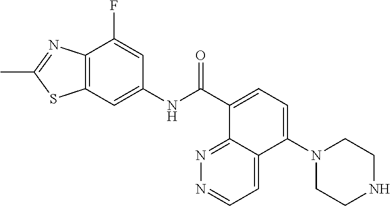 [SMILES: Cc1nc2c(F)cc(NC(=O)c3ccc(N4CCNCC4)c4ccnnc34)cc2s1] |
| 119 | 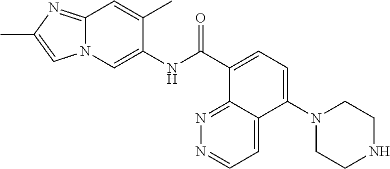 [SMILES: Cc1cn2cc(NC(=O)c3ccc(N4CCNCC4)c4ccnnc34)c(C)cc2n1] |
| 120 | 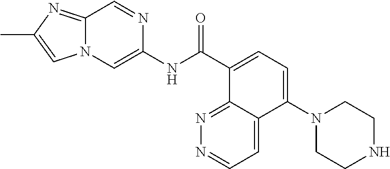 [SMILES: Cc1cn2cc(NC(=O)c3ccc(N4CCNCC4)c4ccnnc34)ncc2n1] |
| 121 | 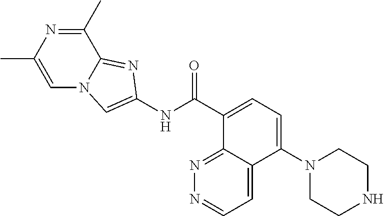 [SMILES: Cc1cn2cc(NC(=O)c3ccc(N4CCNCC4)c4ccnnc34)nc2c(C)n1] |
| 122 | 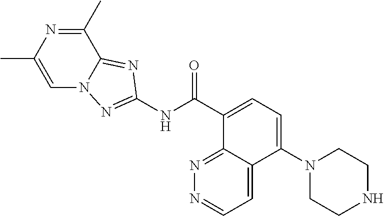 [SMILES: Cc1cn2nc(NC(=O)c3ccc(N4CCNCC4)c4ccnnc34)nc2c(C)n1] |
| 123 | 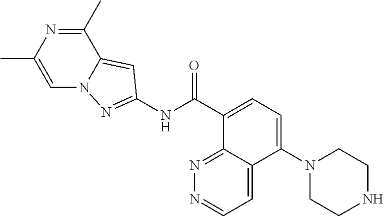 [SMILES: Cc1cn2nc(NC(=O)c3ccc(N4CCNCC4)c4ccnnc34)cc2c(C)n1] |
| 124 | 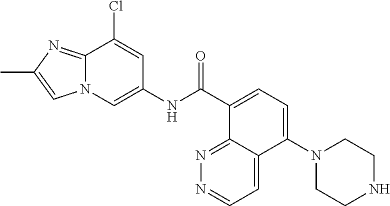 [SMILES: Cc1cn2cc(NC(=O)c3ccc(N4CCNCC4)c4ccnnc34)cc(Cl)c2n1] |
| 125 | 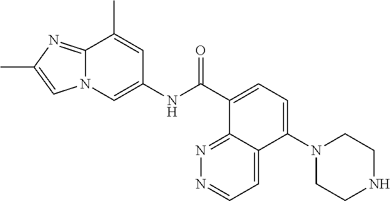 [SMILES: Cc1cn2cc(NC(=O)c3ccc(N4CCNCC4)c4ccnnc34)cc(C)c2n1] |
| 126 | 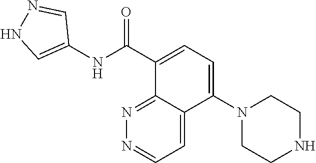 [SMILES: O=C(Nc1cn[nH]c1)c1ccc(N2CCNCC2)c2ccnnc12] |
| 127 | 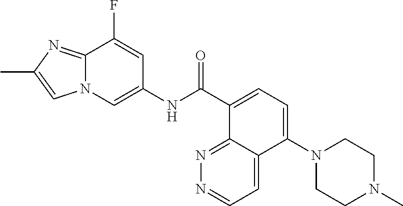 |
| 128 | 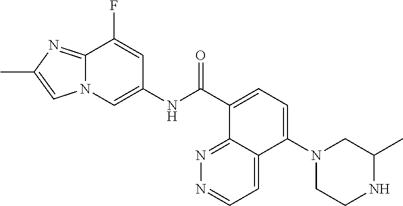 |
| 129 | 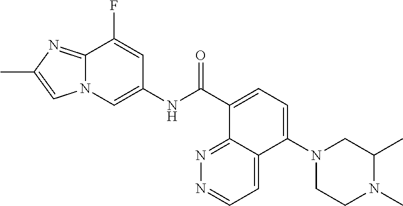 |
| 130 | 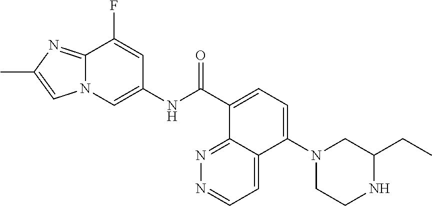 [SMILES: CCC1CN(c2ccc(C(=O)Nc3cc(F)c4nc(C)cn4c3)c3nnccc23)CCN1] |
| 131 | 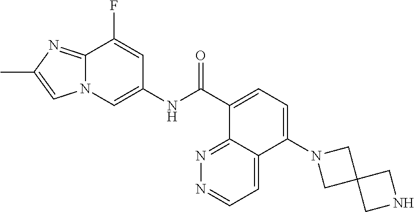 [SMILES: Cc1cn2cc(NC(=O)c3ccc(N4CC5(CNC5)C4)c4ccnnc34)cc(F)c2n1] |
| 132 | 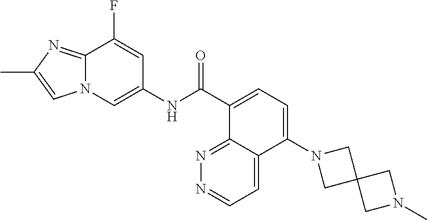 |
| 133 | 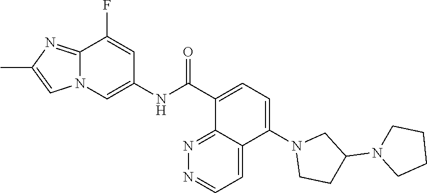 [SMILES: Cc1cn2cc(NC(=O)c3ccc(N4CCC(N5CCCC5)C4)c4ccnnc34)cc(F)c2n1] |
| 134 | 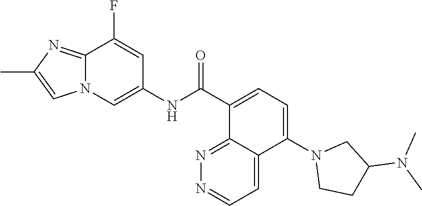 |
| 135 | 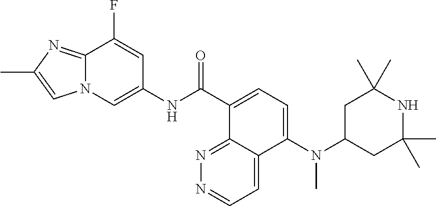 |
| 136 | 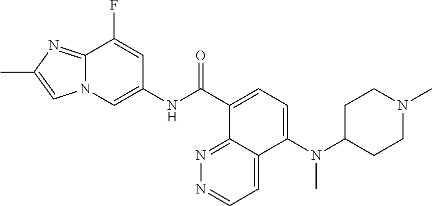 |
| 137 | 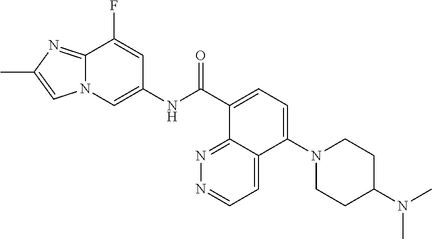 |
| 138 | 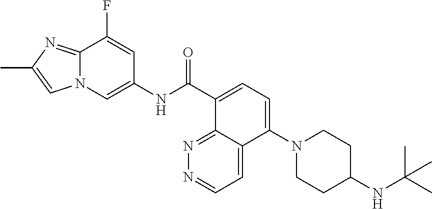 [SMILES: Cc1cn2cc(NC(=O)c3ccc(N4CCC(NC(C)(C)C)CC4)c4ccnnc34)cc(F)c2n1] |
| 139 | 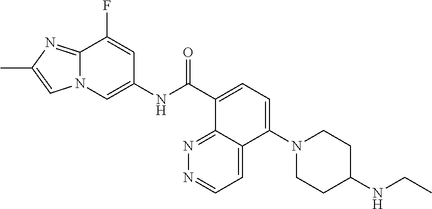 [SMILES: CCNC1CCN(c2ccc(C(=O)Nc3cc(F)c4nc(C)cn4c3)c3nnccc23)CC1] |
| 140 | 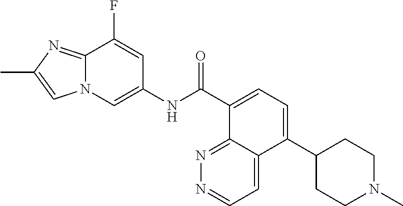 |
| 141 | 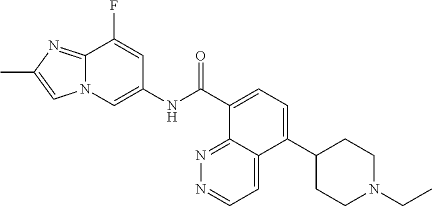 [SMILES: CCN1CCC(c2ccc(C(=O)Nc3cc(F)c4nc(C)cn4c3)c3nnccc23)CC1] |
| 142 | 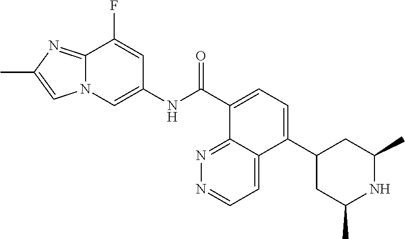 |
| 143 | 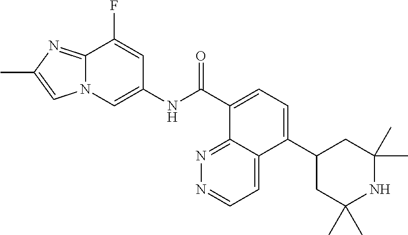 |
| 144 | 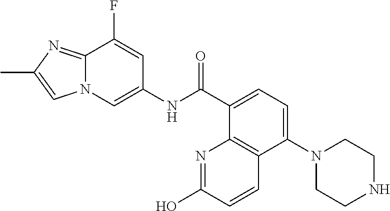 [SMILES: Cc1cn2cc(NC(=O)c3ccc(N4CCNCC4)c4ccc(O)nc34)cc(F)c2n1] |
| 146 | 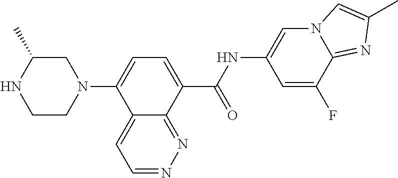 [SMILES: Cc1cn2cc(NC(=O)c3ccc(N4CCN[C@H](C)C4)c4ccnnc34)cc(F)c2n1] |
| 147 | 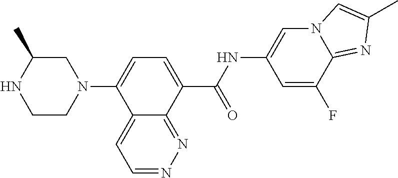 [SMILES: Cc1cn2cc(NC(=O)c3ccc(N4CCN[C@@H](C)C4)c4ccnnc34)cc(F)c2n1] |
| 148 | 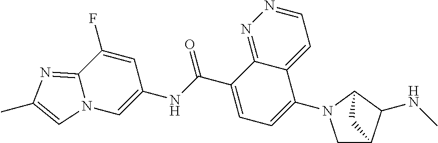 |
| 149 | 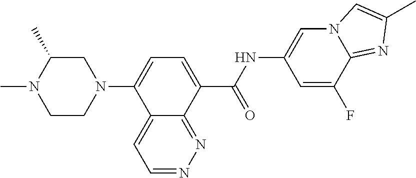 |
| 150 | 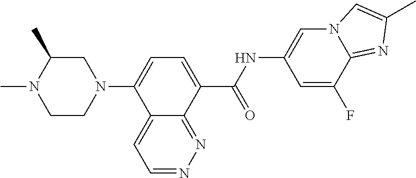 |
| 151 | 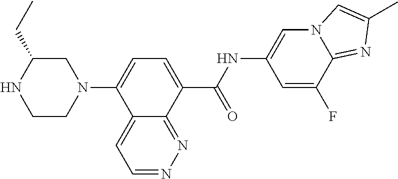 [SMILES: CC[C@@H]1CN(c2ccc(C(=O)Nc3cc(F)c4nc(C)cn4c3)c3nnccc23)CCN1] |
| 152 | 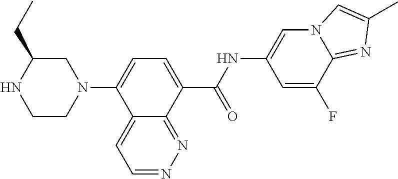 [SMILES: CC[C@H]1CN(c2ccc(C(=O)Nc3cc(F)c4nc(C)cn4c3)c3nnccc23)CCN1] |
| 153 | 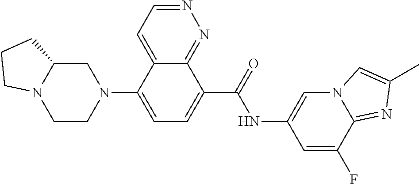 [SMILES: Cc1cn2cc(NC(=O)c3ccc(N4CCN5CCC[C@@H]5C4)c4ccnnc34)cc(F)c2n1] |
| 154 | 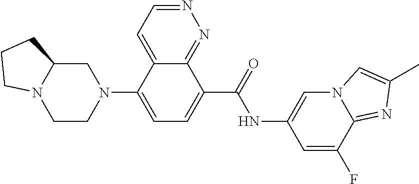 [SMILES: Cc1cn2cc(NC(=O)c3ccc(N4CCN5CCC[C@H]5C4)c4ccnnc34)cc(F)c2n1] |
| 155 | 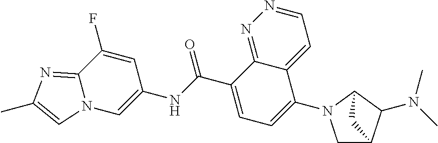 |
| 156 | 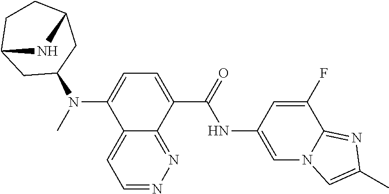 [SMILES: Cc1cn2cc(NC(=O)c3ccc(N(C)[C@@H]4C[C@H]5CC[C@@H](C4)N5)c4ccnnc34)cc(F)c2n1] |
| 157 | 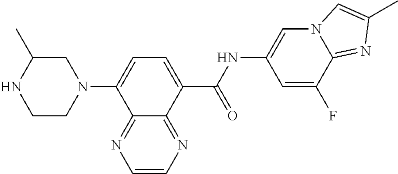 [SMILES: Cc1cn2cc(NC(=O)c3ccc(N4CCNC(C)C4)c4nccnc34)cc(F)c2n1] |
| 158 | 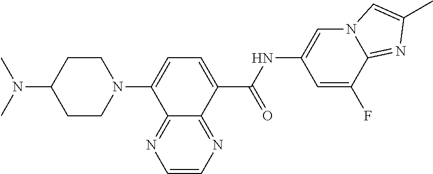 |
| 159 | 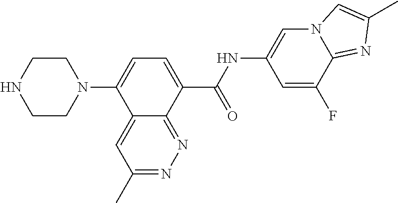 [SMILES: Cc1cc2c(N3CCNCC3)ccc(C(=O)Nc3cc(F)c4nc(C)cn4c3)c2nn1] |
| 160 | 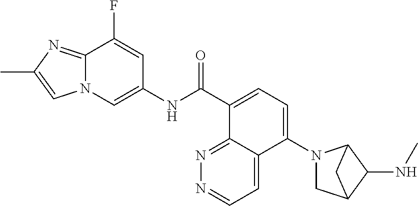 |
| 161 | 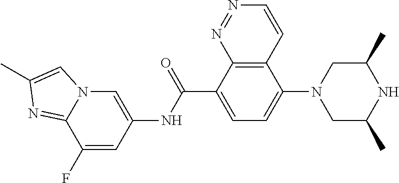 |
| 162 | 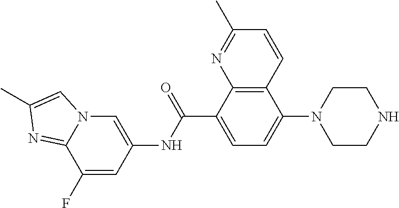 [SMILES: Cc1ccc2c(N3CCNCC3)ccc(C(=O)Nc3cc(F)c4nc(C)cn4c3)c2n1] |
| 163 | 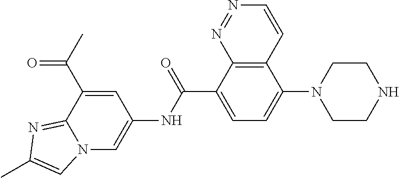 [SMILES: CC(=O)c1cc(NC(=O)c2ccc(N3CCNCC3)c3ccnnc23)cn2cc(C)nc12] |
| 164 | 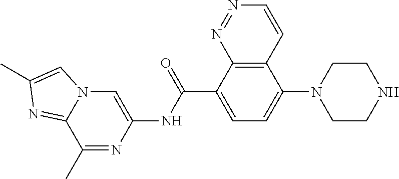 [SMILES: Cc1cn2cc(NC(=O)c3ccc(N4CCNCC4)c4ccnnc34)nc(C)c2n1] |
| 165 | 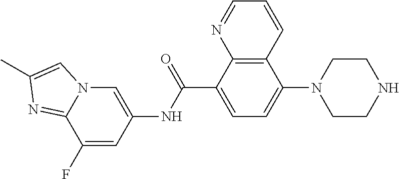 [SMILES: Cc1cn2cc(NC(=O)c3ccc(N4CCNCC4)c4cccnc34)cc(F)c2n1] |
| 166 | 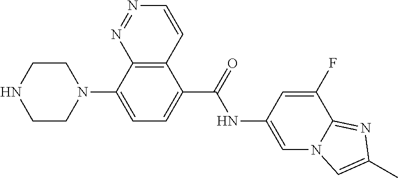 [SMILES: Cc1cn2cc(NC(=O)c3ccc(N4CCNCC4)c4nnccc34)cc(F)c2n1] |
| 167 | 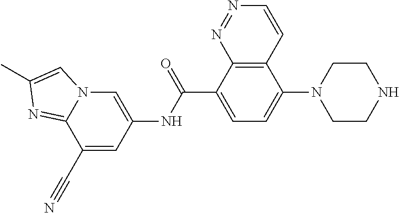 [SMILES: Cc1cn2cc(NC(=O)c3ccc(N4CCNCC4)c4ccnnc34)cc(C#N)c2n1] |
| 168 | 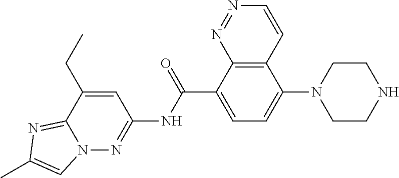 [SMILES: CCc1cc(NC(=O)c2ccc(N3CCNCC3)c3ccnnc23)nn2cc(C)nc12] |
| 169 | 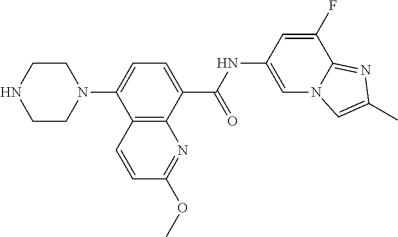 [SMILES: COc1ccc2c(N3CCNCC3)ccc(C(=O)Nc3cc(F)c4nc(C)cn4c3)c2n1] |
| 170 | 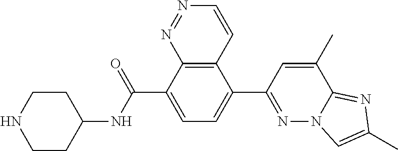 [SMILES: Cc1cn2nc(-c3ccc(C(=O)NC4CCNCC4)c4nnccc34)cc(C)c2n1] |
| 171 | 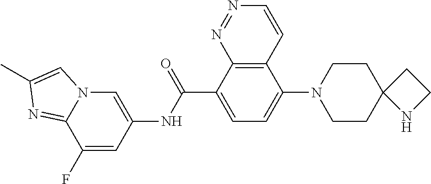 [SMILES: Cc1cn2cc(NC(=O)c3ccc(N4CCC5(CCN5)CC4)c4ccnnc34)cc(F)c2n1] |
| 172 | 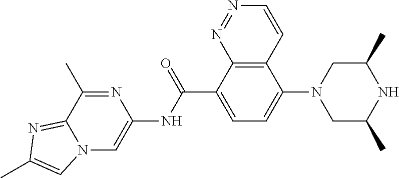 |
| 173 | 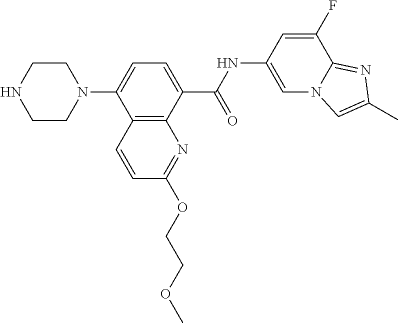 [SMILES: COCCOc1ccc2c(N3CCNCC3)ccc(C(=O)Nc3cc(F)c4nc(C)cn4c3)c2n1] |
| 176 | 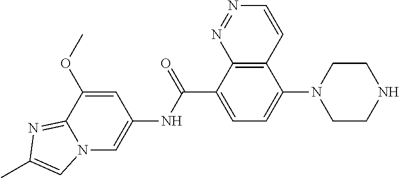 [SMILES: COc1cc(NC(=O)c2ccc(N3CCNCC3)c3ccnnc23)cn2cc(C)nc12] |
| 177 | 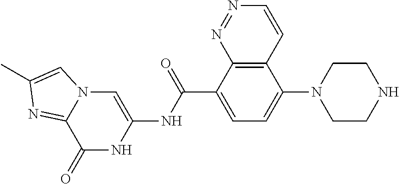 [SMILES: Cc1cn2cc(NC(=O)c3ccc(N4CCNCC4)c4ccnnc34)[nH]c(=O)c2n1] |
| 178 | 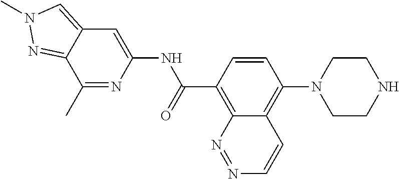 [SMILES: Cc1nc(NC(=O)c2ccc(N3CCNCC3)c3ccnnc23)cc2cn(C)nc12] |
| 179 | 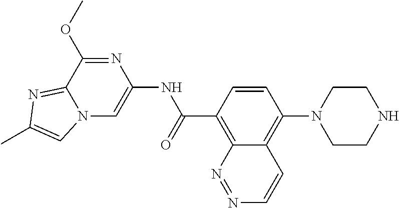 [SMILES: COc1nc(NC(=O)c2ccc(N3CCNCC3)c3ccnnc23)cn2cc(C)nc12] |
| 180 | 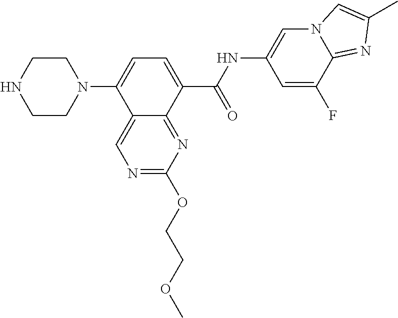 [SMILES: COCCOc1ncc2c(N3CCNCC3)ccc(C(=O)Nc3cc(F)c4nc(C)cn4c3)c2n1] |
| 181 | 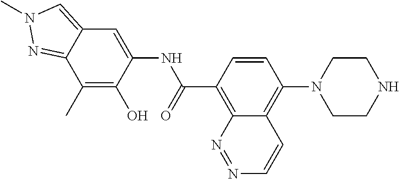 [SMILES: Cc1c(O)c(NC(=O)c2ccc(N3CCNCC3)c3ccnnc23)cc2cn(C)nc12] |
| 182 | 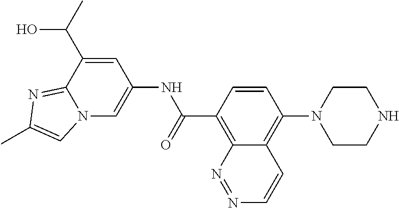 [SMILES: Cc1cn2cc(NC(=O)c3ccc(N4CCNCC4)c4ccnnc34)cc(C(C)O)c2n1] |
| 183 | 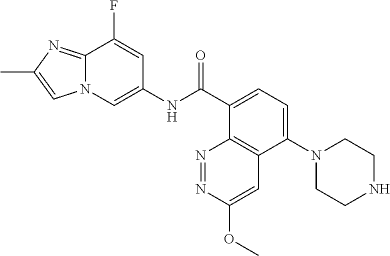 [SMILES: COc1cc2c(N3CCNCC3)ccc(C(=O)Nc3cc(F)c4nc(C)cn4c3)c2nn1] |
| 185 | 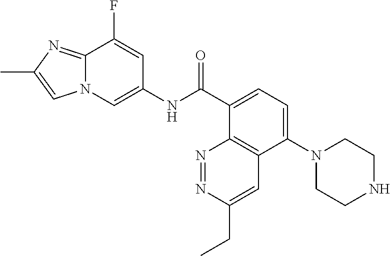 [SMILES: CCc1cc2c(N3CCNCC3)ccc(C(=O)Nc3cc(F)c4nc(C)cn4c3)c2nn1] |
| 186 | 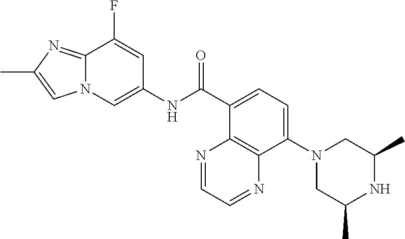 |
| 187 | 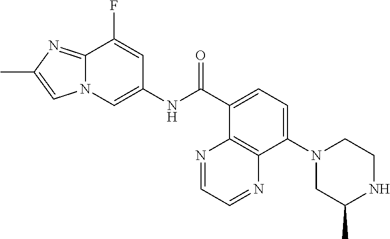 [SMILES: Cc1cn2cc(NC(=O)c3ccc(N4CCN[C@@H](C)C4)c4nccnc34)cc(F)c2n1] |
| 188 | 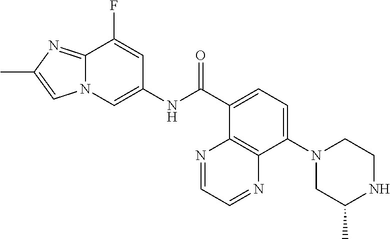 [SMILES: Cc1cn2cc(NC(=O)c3ccc(N4CCN[C@H](C)C4)c4nccnc34)cc(F)c2n1] |
| 189 | 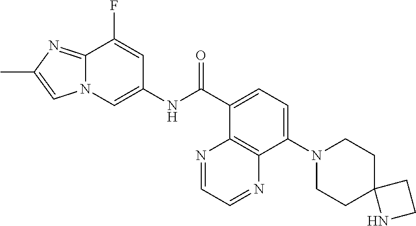 [SMILES: Cc1cn2cc(NC(=O)c3ccc(N4CCC5(CCN5)CC4)c4nccnc34)cc(F)c2n1] |
| 190 | 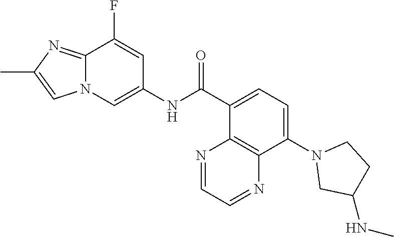 |
| 191 | 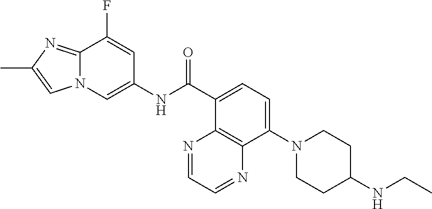 [SMILES: CCNC1CCN(c2ccc(C(=O)Nc3cc(F)c4nc(C)cn4c3)c3nccnc23)CC1] |
| 192 | 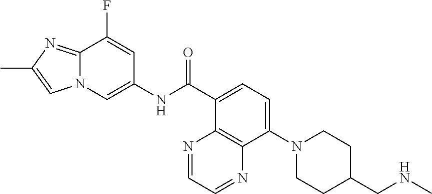 |
| 193 | 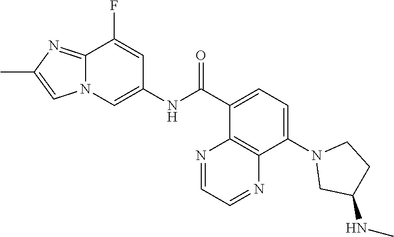 |
| 194 | 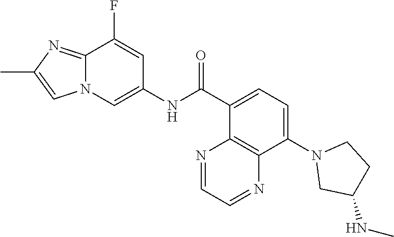 |
| 195 | 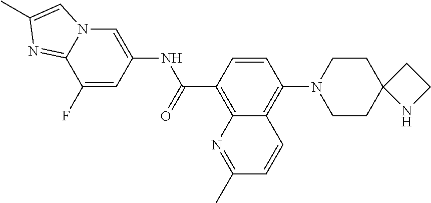 [SMILES: Cc1ccc2c(N3CCC4(CCN4)CC3)ccc(C(=O)Nc3cc(F)c4nc(C)cn4c3)c2n1] |
| 196 | 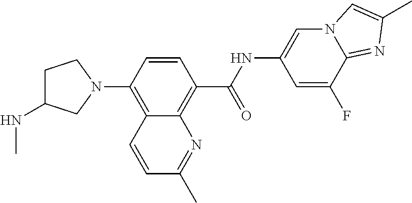 [SMILES: CNC1CCN(c2ccc(C(=O)Nc3cc(F)c4nc(C)cn4c3)c3nc(C)ccc23)C1] |
| 197 | 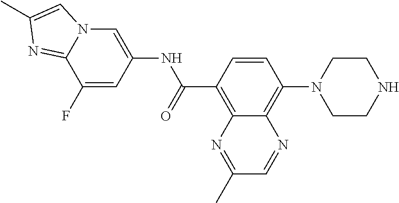 [SMILES: Cc1cnc2c(N3CCNCC3)ccc(C(=O)Nc3cc(F)c4nc(C)cn4c3)c2n1] |
| 198 | 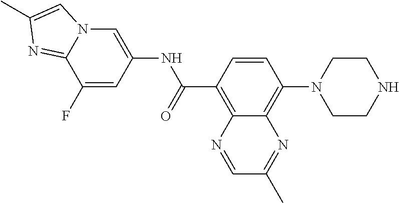 [SMILES: Cc1cnc2c(C(=O)Nc3cc(F)c4nc(C)cn4c3)ccc(N3CCNCC3)c2n1] |
| 199 | 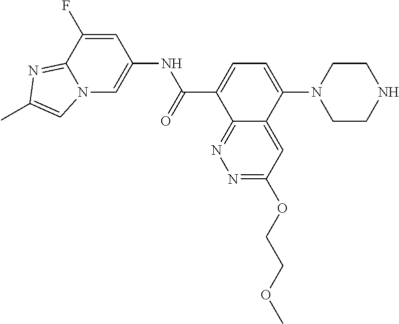 [SMILES: COCCOc1cc2c(N3CCNCC3)ccc(C(=O)Nc3cc(F)c4nc(C)cn4c3)c2nn1] |
| 200 | 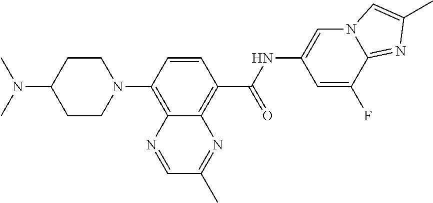 |
| 201 | 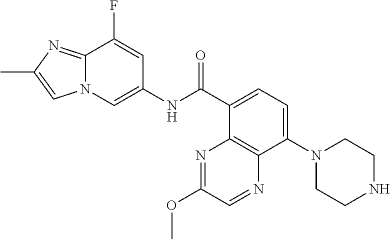 [SMILES: COc1cnc2c(N3CCNCC3)ccc(C(=O)Nc3cc(F)c4nc(C)cn4c3)c2n1] |
| 202 | 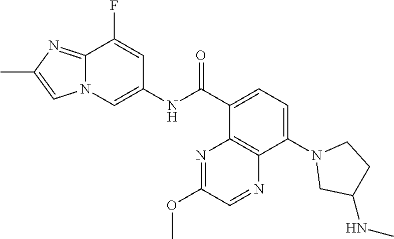 |
| 203 | 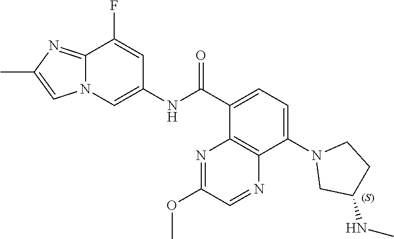 |
| 204 | 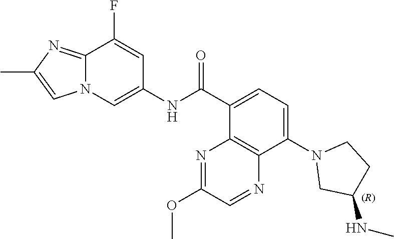 |
| 205 | 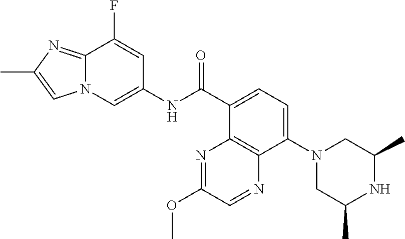 |
| 206 | 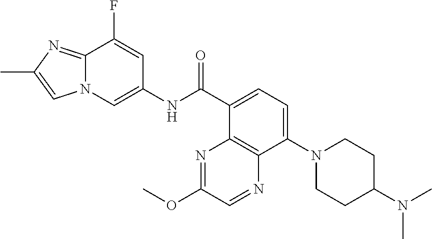 |
| 207 | 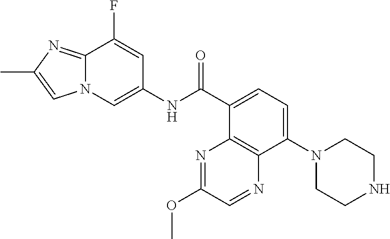 [SMILES: COc1cnc2c(N3CCNCC3)ccc(C(=O)Nc3cc(F)c4nc(C)cn4c3)c2n1] |
| 208 | 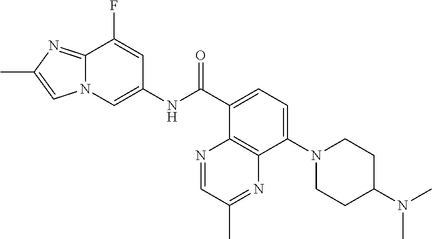 |
| 209 | 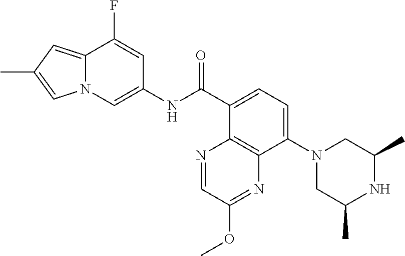 |
| 210 | 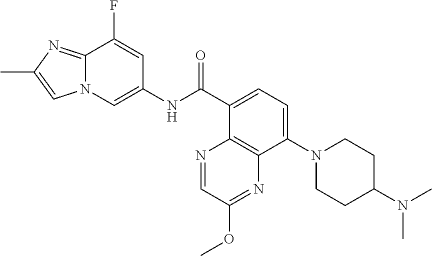 |
| 211 | 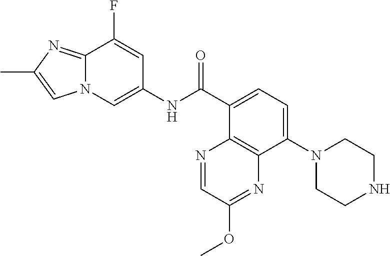 [SMILES: COc1cnc2c(C(=O)Nc3cc(F)c4nc(C)cn4c3)ccc(N3CCNCC3)c2n1] |
| 212 | 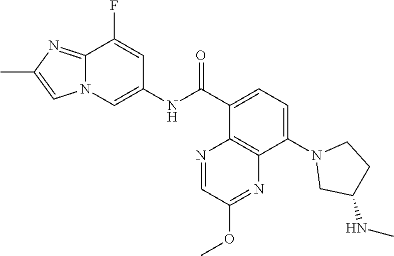 |
| 213 | 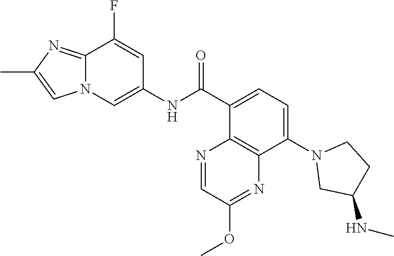 |
| 214 | 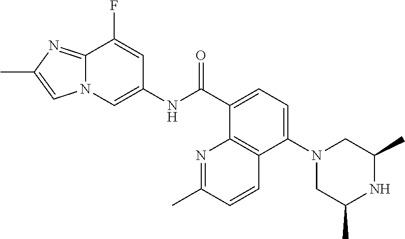 |
| 215 | 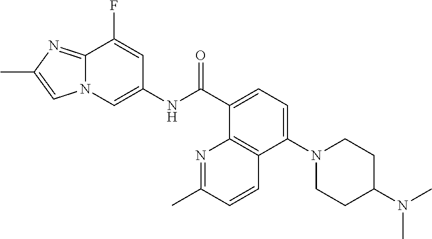 |
| 216 | 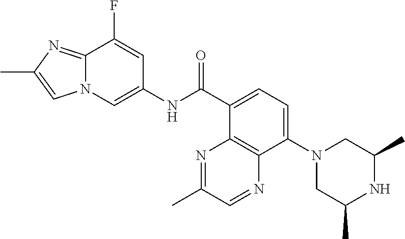 |
| 217 | 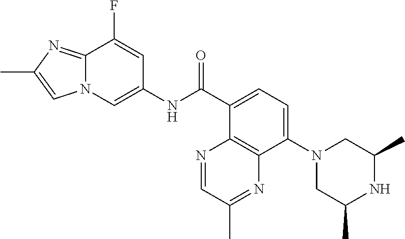 |
| 218 | 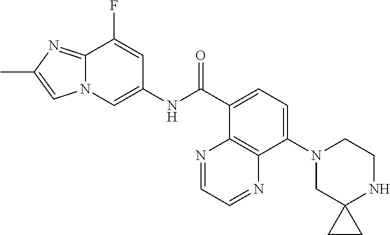 [SMILES: Cc1cn2cc(NC(=O)c3ccc(N4CCNC5(CC5)C4)c4nccnc34)cc(F)c2n1] |
| 219 | 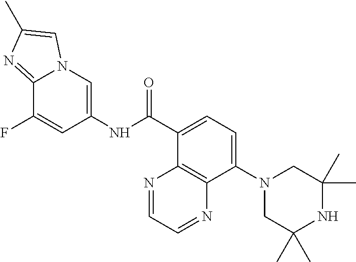 [SMILES: Cc1cn2cc(NC(=O)c3ccc(N4CC(C)(C)NC(C)(C)C4)c4nccnc34)cc(F)c2n1] |
| 220 | 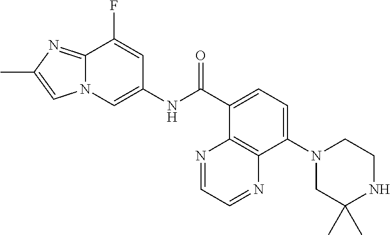 [SMILES: Cc1cn2cc(NC(=O)c3ccc(N4CCNC(C)(C)C4)c4nccnc34)cc(F)c2n1] |
| 221 | 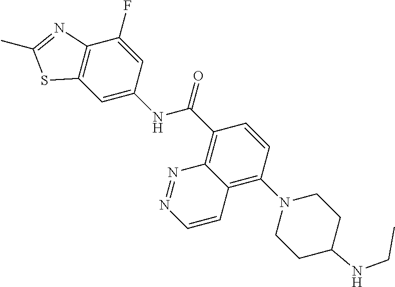 [SMILES: CCNC1CCN(c2ccc(C(=O)Nc3cc(F)c4nc(C)sc4c3)c3nnccc23)CC1] |
| 222 | 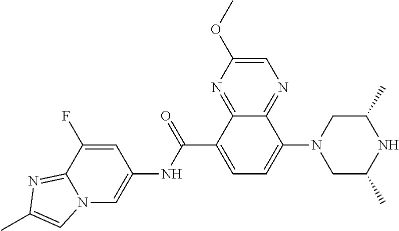 |
| 223 | 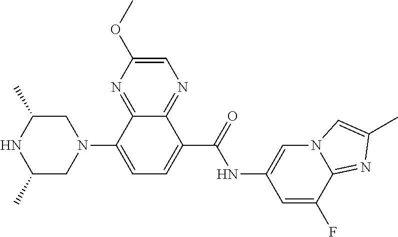 [SMILES: COc1cnc2c(C(=O)Nc3cc(F)c4nc(C)cn4c3)ccc(N3C[C@@H](C)N[C@@H](C)C3)c2n1] |
| 224 | 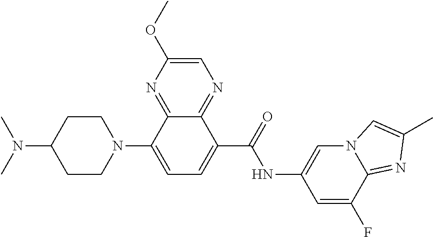 |
| 225 | 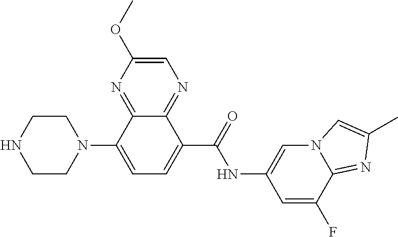 [SMILES: COc1cnc2c(C(=O)Nc3cc(F)c4nc(C)cn4c3)ccc(N3CCNCC3)c2n1] |
| 226 | 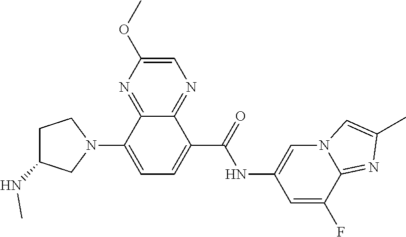 [SMILES: CN[C@@H]1CCN(c2ccc(C(=O)Nc3cc(F)c4nc(C)cn4c3)c3ncc(OC)nc23)C1] |
| 227 | 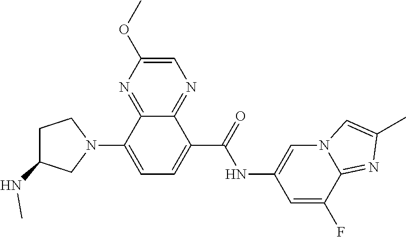 [SMILES: CN[C@H]1CCN(c2ccc(C(=O)Nc3cc(F)c4nc(C)cn4c3)c3ncc(OC)nc23)C1] |
| 228 | 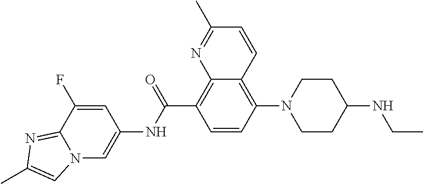 [SMILES: CCNC1CCN(c2ccc(C(=O)Nc3cc(F)c4nc(C)cn4c3)c3nc(C)ccc23)CC1] |
| 229 | 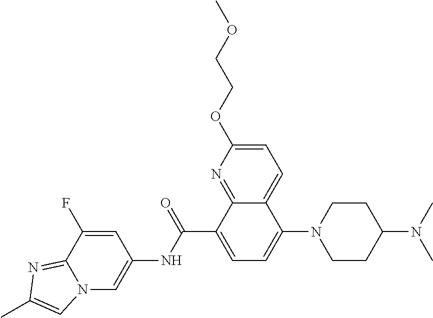 |
| 230 | 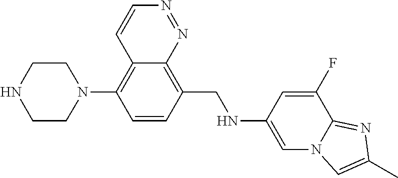 [SMILES: Cc1cn2cc(NCc3ccc(N4CCNCC4)c4ccnnc34)cc(F)c2n1] |
| 231 | 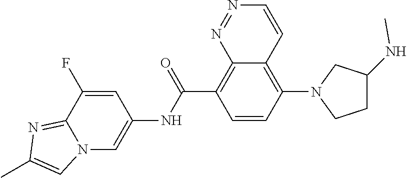 [SMILES: CNC1CCN(c2ccc(C(=O)Nc3cc(F)c4nc(C)cn4c3)c3nnccc23)C1] |
| 232 | 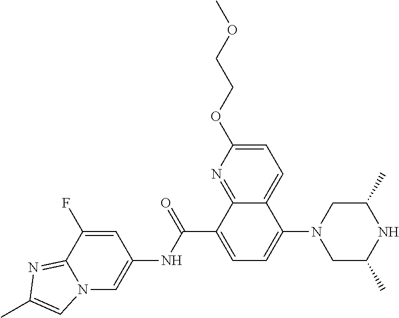 |
| 233 | 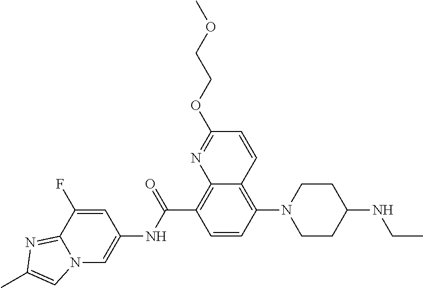 [SMILES: CCNC1CCN(c2ccc(C(=O)Nc3cc(F)c4nc(C)cn4c3)c3nc(OCCOC)ccc23)CC1] |
| 234 | 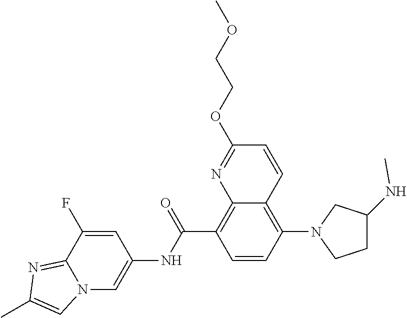 [SMILES: CNC1CCN(c2ccc(C(=O)Nc3cc(F)c4nc(C)cn4c3)c3nc(OCCOC)ccc23)C1] |
| 235 | 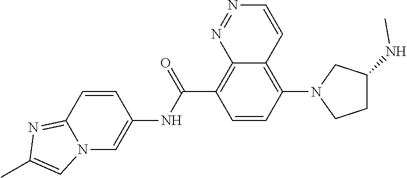 [SMILES: CN[C@@H]1CCN(c2ccc(C(=O)Nc3ccc4nc(C)cn4c3)c3nnccc23)C1] |
| 236 | 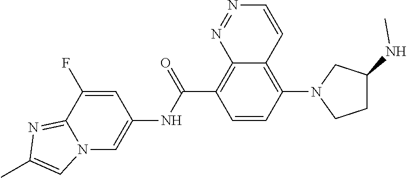 [SMILES: CN[C@H]1CCN(c2ccc(C(=O)Nc3cc(F)c4nc(C)cn4c3)c3nnccc23)C1] |
| 237 | 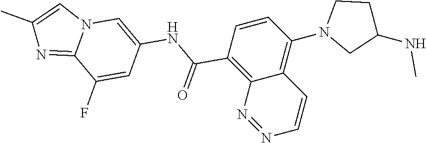 |
| 238 | 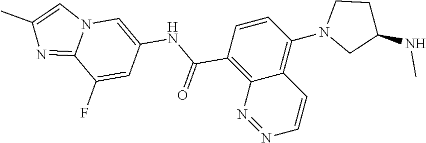 |
| 239 | 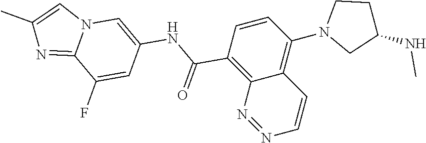 |
| 240 | 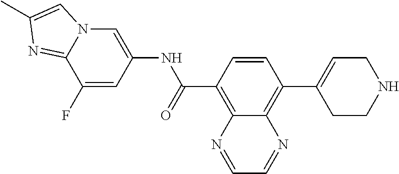 [SMILES: Cc1cn2cc(NC(=O)c3ccc(C4=CCNCC4)c4nccnc34)cc(F)c2n1] |
| 241 | 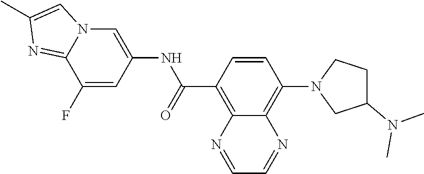 |
| 242 | 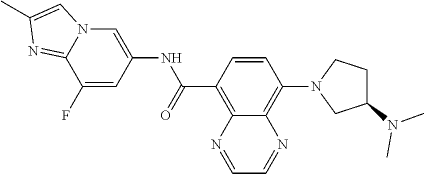 |
| 243 | 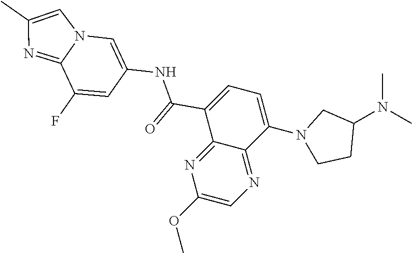 |
| 244 | 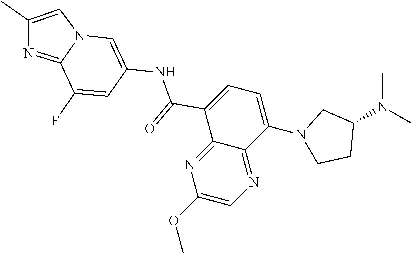 |
| 245 | 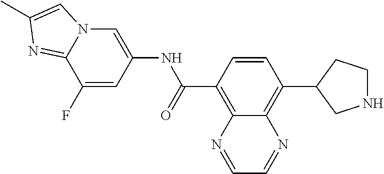 [SMILES: Cc1cn2cc(NC(=O)c3ccc(C4CCNC4)c4nccnc34)cc(F)c2n1] |
| 246 | 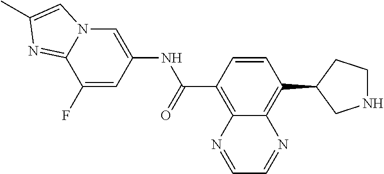 [SMILES: Cc1cn2cc(NC(=O)c3ccc([C@H]4CCNC4)c4nccnc34)cc(F)c2n1] |
| 247 | 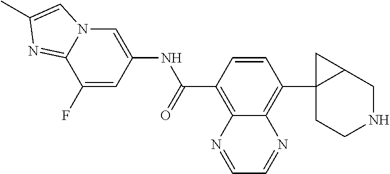 [SMILES: Cc1cn2cc(NC(=O)c3ccc(C45CCNCC4C5)c4nccnc34)cc(F)c2n1] |
| 248 | 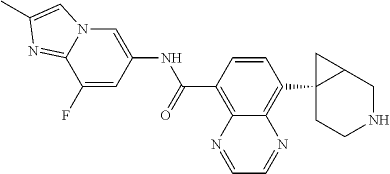 [SMILES: Cc1cn2cc(NC(=O)c3ccc([C@@]45CCNCC4C5)c4nccnc34)cc(F)c2n1] |
| 249 | 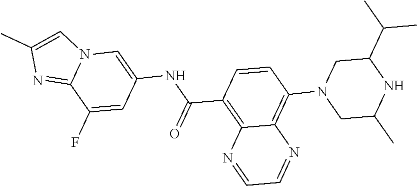 [SMILES: Cc1cn2cc(NC(=O)c3ccc(N4CC(C)NC(C(C)C)C4)c4nccnc34)cc(F)c2n1] |
| 250 | 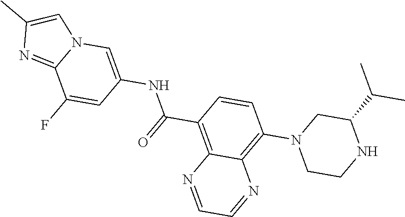 [SMILES: Cc1cn2cc(NC(=O)c3ccc(N4CCN[C@@H](C(C)C)C4)c4nccnc34)cc(F)c2n1] |
| 251 | 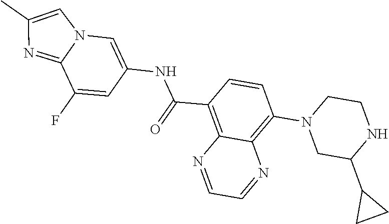 [SMILES: Cc1cn2cc(NC(=O)c3ccc(N4CCNC(C5CC5)C4)c4nccnc34)cc(F)c2n1] |
| 252 | 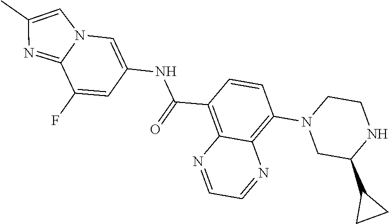 [SMILES: Cc1cn2cc(NC(=O)c3ccc(N4CCN[C@@H](C5CC5)C4)c4nccnc34)cc(F)c2n1] |
| 253 | 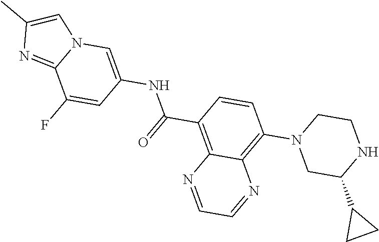 [SMILES: Cc1cn2cc(NC(=O)c3ccc(N4CCN[C@H](C5CC5)C4)c4nccnc34)cc(F)c2n1] |
| 254 | 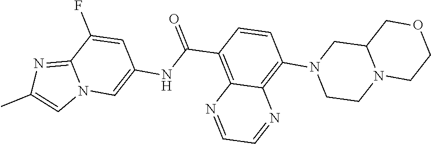 [SMILES: Cc1cn2cc(NC(=O)c3ccc(N4CCN5CCOCC5C4)c4nccnc34)cc(F)c2n1] |
| 255 | 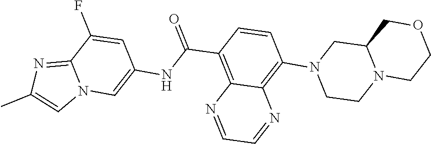 [SMILES: Cc1cn2cc(NC(=O)c3ccc(N4CCN5CCOC[C@@H]5C4)c4nccnc34)cc(F)c2n1] |
| 256 | 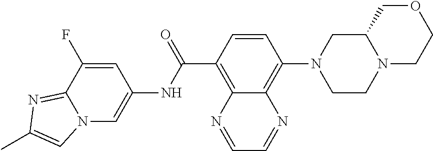 [SMILES: Cc1cn2cc(NC(=O)c3ccc(N4CCN5CCOC[C@H]5C4)c4nccnc34)cc(F)c2n1] |
| 257 | 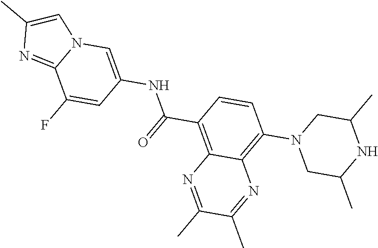 |
| 258 | 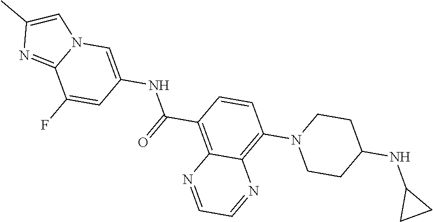 [SMILES: Cc1cn2cc(NC(=O)c3ccc(N4CCC(NC5CC5)CC4)c4nccnc34)cc(F)c2n1] |
| 259 | 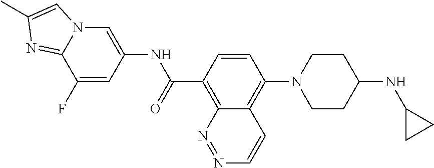 [SMILES: Cc1cn2cc(NC(=O)c3ccc(N4CCC(NC5CC5)CC4)c4ccnnc34)cc(F)c2n1] |
| 260 | 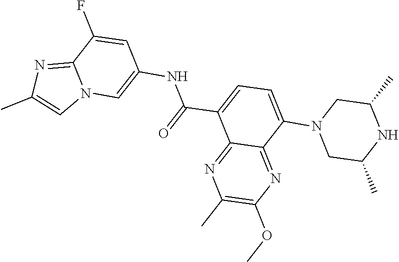 |
| 261 | 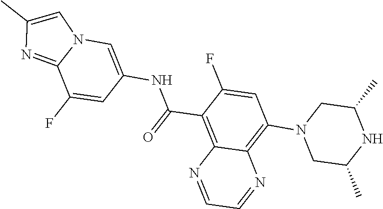 |
| 262 | 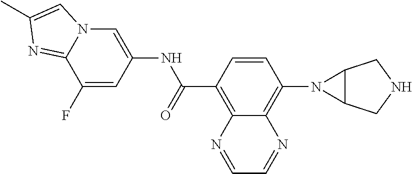 [SMILES: Cc1cn2cc(NC(=O)c3ccc(N4C5CNCC54)c4nccnc34)cc(F)c2n1] |
| 264 | 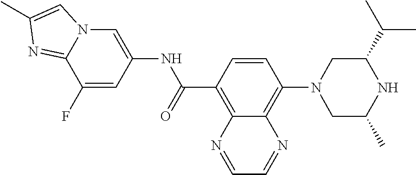 [SMILES: Cc1cn2cc(NC(=O)c3ccc(N4C[C@@H](C)N[C@@H](C(C)C)C4)c4nccnc34)cc(F)c2n1] |
| 265 | 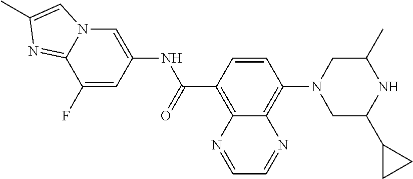 [SMILES: Cc1cn2cc(NC(=O)c3ccc(N4CC(C)NC(C5CC5)C4)c4nccnc34)cc(F)c2n1] |
| 266 | 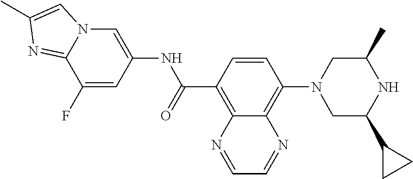 [SMILES: Cc1cn2cc(NC(=O)c3ccc(N4C[C@@H](C)N[C@@H](C5CC5)C4)c4nccnc34)cc(F)c2n1] |
| 267 | 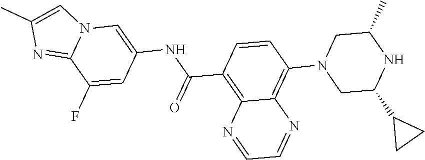 [SMILES: Cc1cn2cc(NC(=O)c3ccc(N4C[C@@H](C5CC5)N[C@@H](C)C4)c4nccnc34)cc(F)c2n1] |
| 268 | 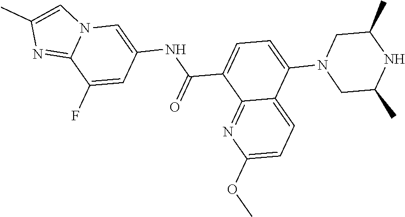 |
| 269 | 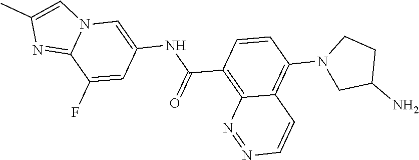 [SMILES: Cc1cn2cc(NC(=O)c3ccc(N4CCC(N)C4)c4ccnnc34)cc(F)c2n1] |
| 270 | 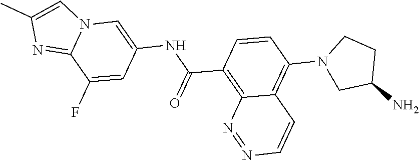 [SMILES: Cc1cn2cc(NC(=O)c3ccc(N4CC[C@@H](N)C4)c4ccnnc34)cc(F)c2n1] |
| 271 | 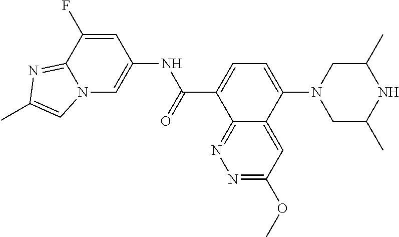 |
| 273 | 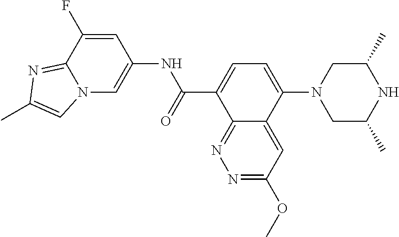 |
| 274 | 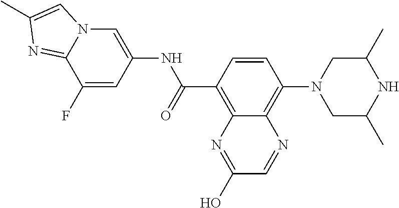 |
| 275 | 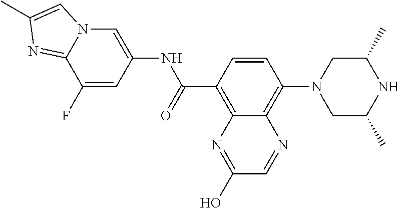 |
| 276 | 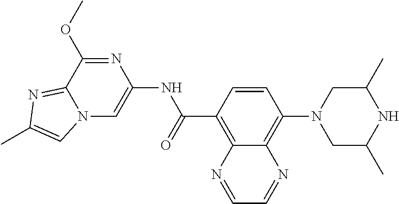 |
| 277 | 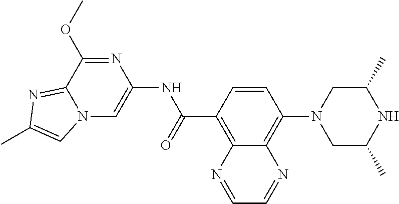 |
| 278 | 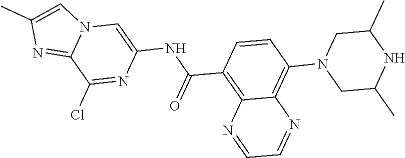 |
| 279 | 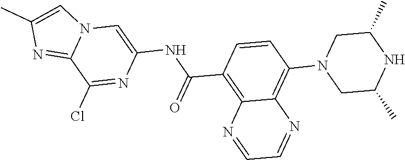 |
| 280 | 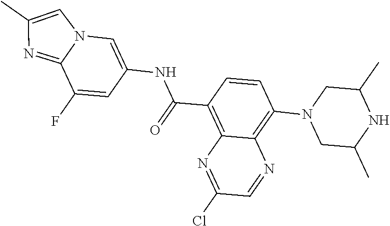 |
| 281 | 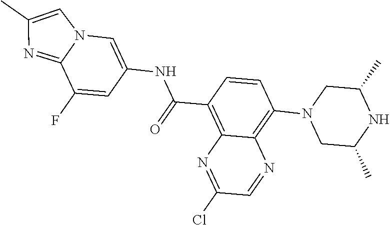 |
| 282 | 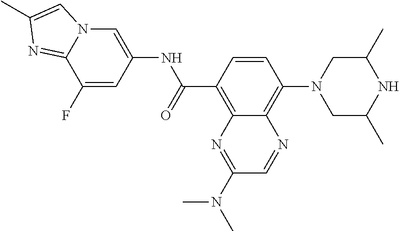 |
| 283 | 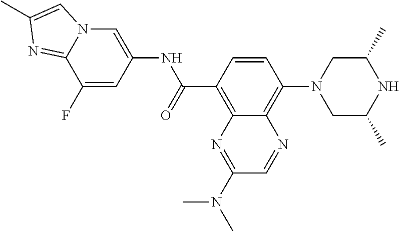 |
| 285 | 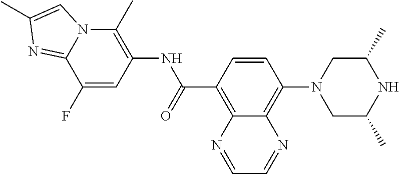 |
| 286 | 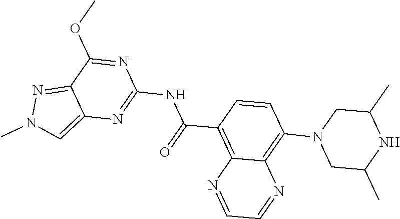 |
| 287 | 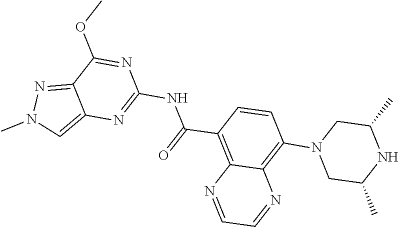 |
| 288 | 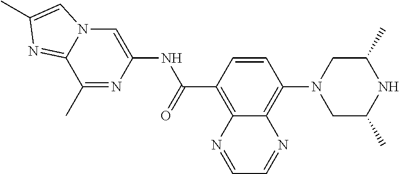 |
| 289 | 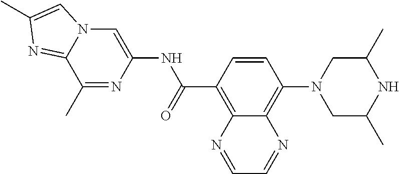 |
| 290 | 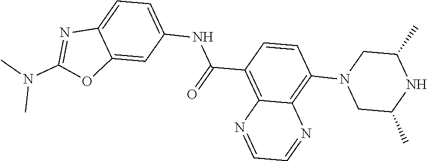 |
| 291 | 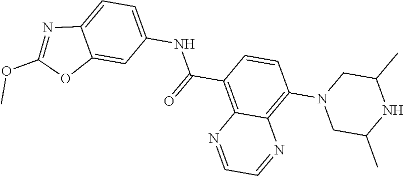 |
| 292 | 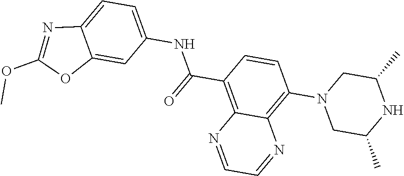 |
| 293 | 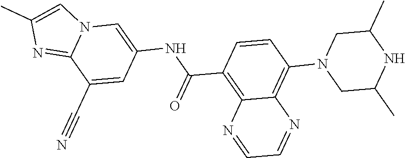 |
| 294 | 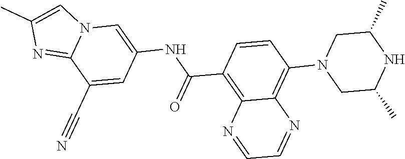 |
| 295 | 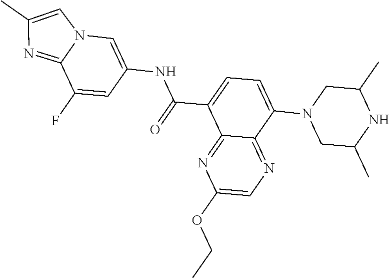 |
| 296 | 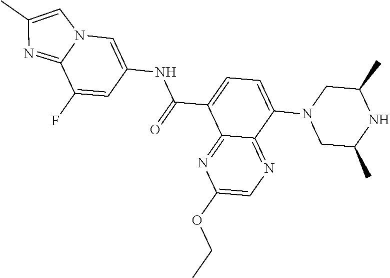 |
| 297 | 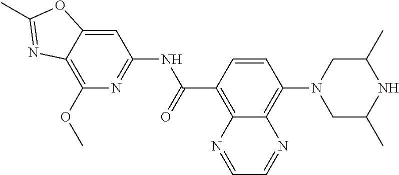 |
| 298 | 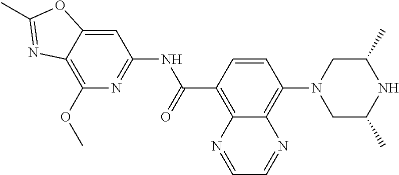 |
| 299 | 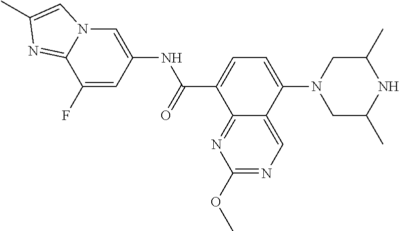 |
| 300 | 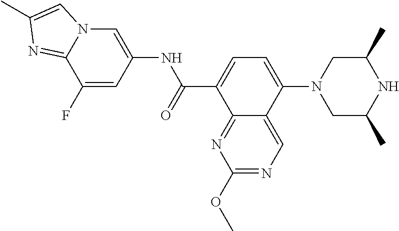 |
| 301 | 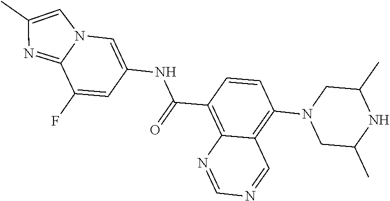 |
| 302 | 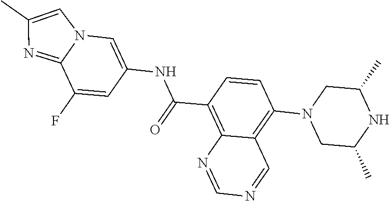 |
| 303 | 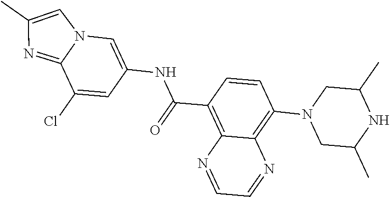 |
| 304 | 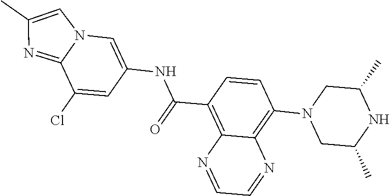 |
| 305 | 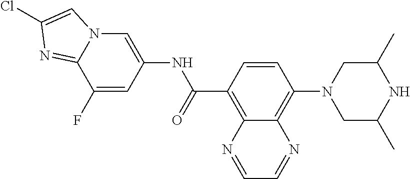 [SMILES: CC1CN(c2ccc(C(=O)Nc3cc(F)c4nc(Cl)cn4c3)c3nccnc23)CC(C)N1] |
| 306 | 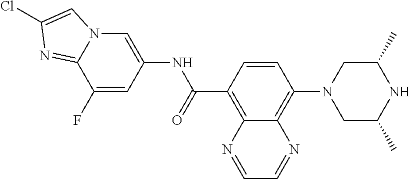 [SMILES: C[C@@H]1CN(c2ccc(C(=O)Nc3cc(F)c4nc(Cl)cn4c3)c3nccnc23)C[C@H](C)N1] |
| 307 | 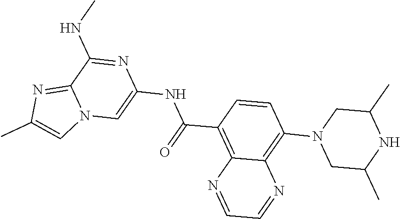 |
| 308 | 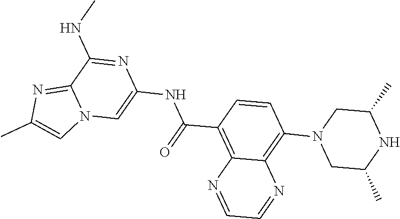 |
| 309 | 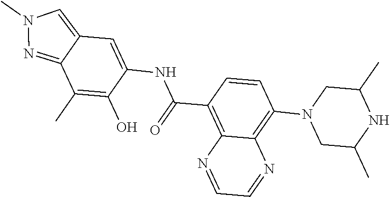 |
| 310 | 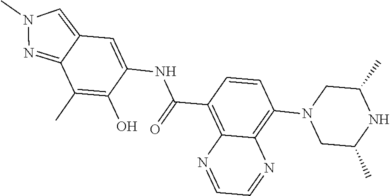 |
| 311 | 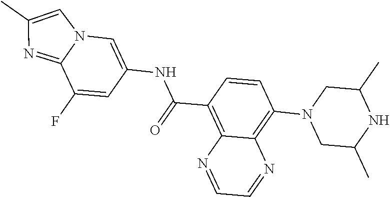 |
| 312 | 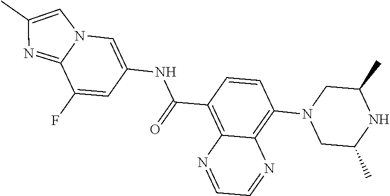 |
| 313 | 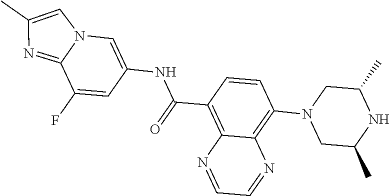 |
| 314 | 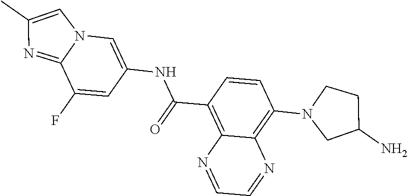 [SMILES: Cc1cn2cc(NC(=O)c3ccc(N4CCC(N)C4)c4nccnc34)cc(F)c2n1] |
| 315 | 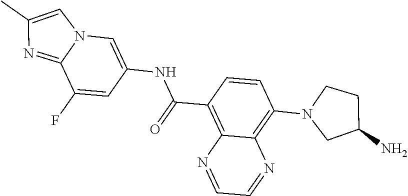 [SMILES: Cc1cn2cc(NC(=O)c3ccc(N4CC[C@@H](N)C4)c4nccnc34)cc(F)c2n1] |
| 316 | 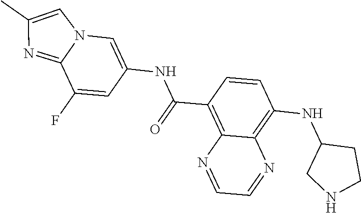 [SMILES: Cc1cn2cc(NC(=O)c3ccc(NC4CCNC4)c4nccnc34)cc(F)c2n1] |
| 318 | 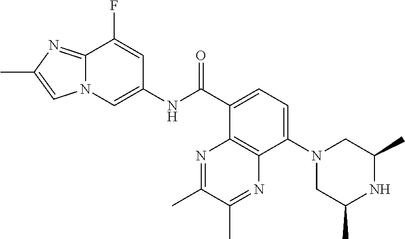 |
| 319 | 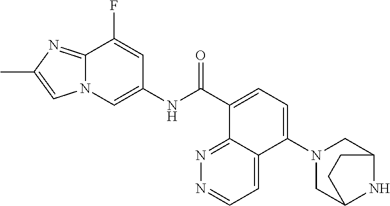 [SMILES: Cc1cn2cc(NC(=O)c3ccc(N4CC5CCC(C4)N5)c4ccnnc34)cc(F)c2n1] |
| 320 | 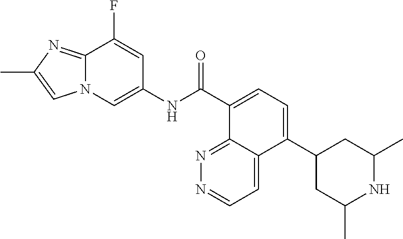 |
| 321 | 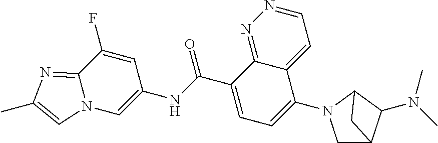 |
| 322 | 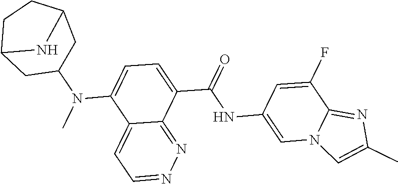 [SMILES: Cc1cn2cc(NC(=O)c3ccc(N(C)C4CC5CCC(C4)N5)c4ccnnc34)cc(F)c2n1] |
| 323 | 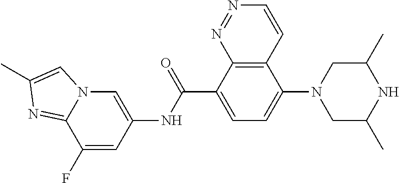 |
| 324 | 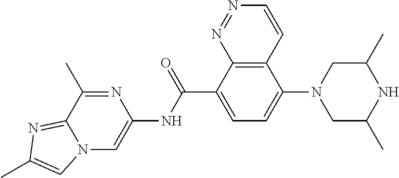 |
| 325 | 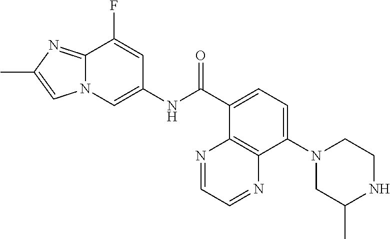 [SMILES: Cc1cn2cc(NC(=O)c3ccc(N4CCNC(C)C4)c4nccnc34)cc(F)c2n1] |
| 326 | 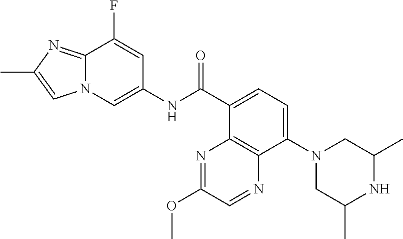 |
| 327 | 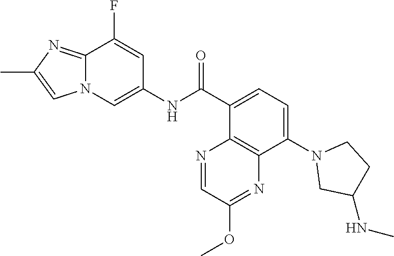 |
| 328 | 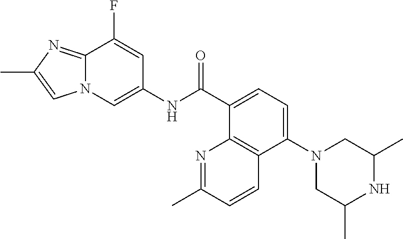 |
| 329 | 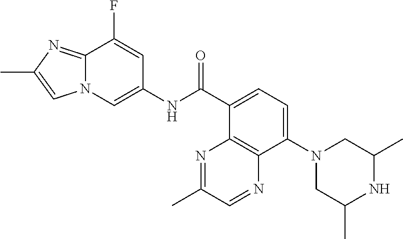 |
| 330 | 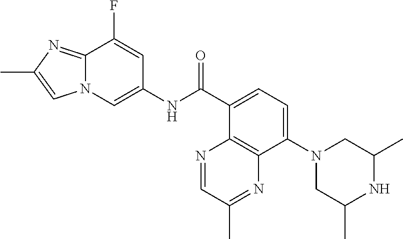 |
| 331 | 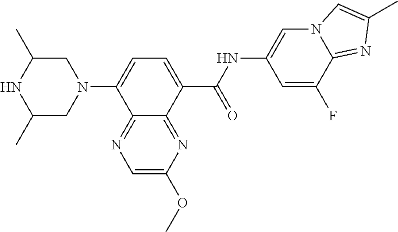 [SMILES: COc1cnc2c(N3CC(C)NC(C)C3)ccc(C(=O)Nc3cc(F)c4nc(C)cn4c3)c2n1] |
| 332 | 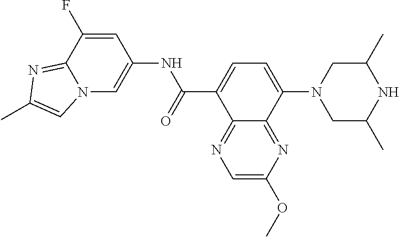 |
| 333 | 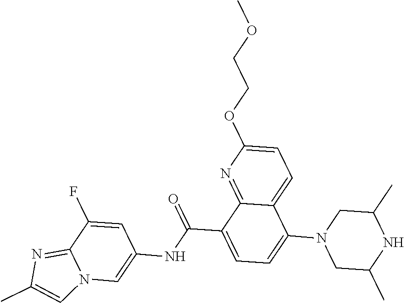 |
| 334 | 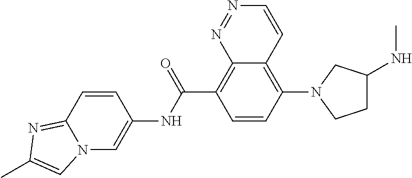 |
| 335 | 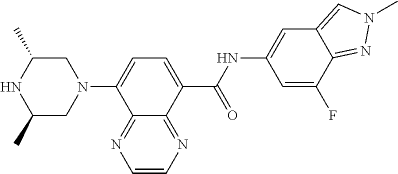 [SMILES: C[C@@H]1CN(c2ccc(C(=O)Nc3cc(F)c4nn(C)cc4c3)c3nccnc23)C[C@@H](C)N1] |
| 339 | 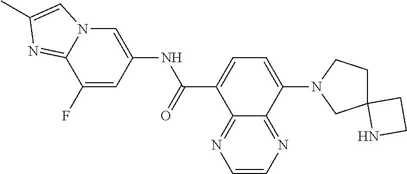 [SMILES: Cc1cn2cc(NC(=O)c3ccc(N4CCC5(CCN5)C4)c4nccnc34)cc(F)c2n1] |
| 340 | 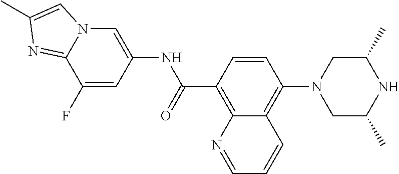 |
| 341 | 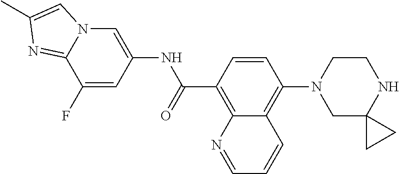 [SMILES: Cc1cn2cc(NC(=O)c3ccc(N4CCNC5(CC5)C4)c4cccnc34)cc(F)c2n1] |
| 342 | 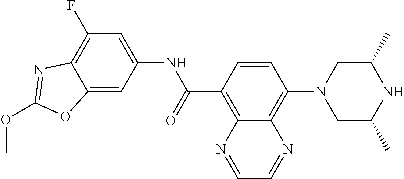 [SMILES: COc1nc2c(F)cc(NC(=O)c3ccc(N4C[C@@H](C)N[C@@H](C)C4)c4nccnc34)cc2o1] |
| 343 | 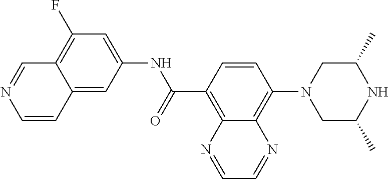 [SMILES: C[C@@H]1CN(c2ccc(C(=O)Nc3cc(F)c4cnccc4c3)c3nccnc23)C[C@H](C)N1] |
| 344 | 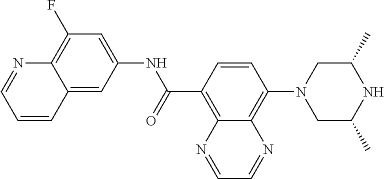 [SMILES: C[C@@H]1CN(c2ccc(C(=O)Nc3cc(F)c4ncccc4c3)c3nccnc23)C[C@H](C)N1] |
| 345 | 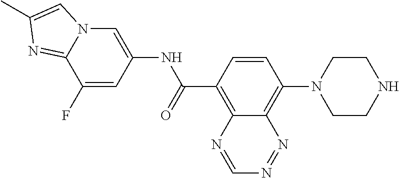 [SMILES: Cc1cn2cc(NC(=O)c3ccc(N4CCNCC4)c4nncnc34)cc(F)c2n1] |
| 346 | 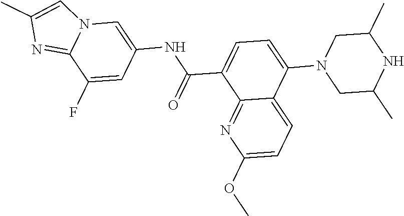 |
| 350 | 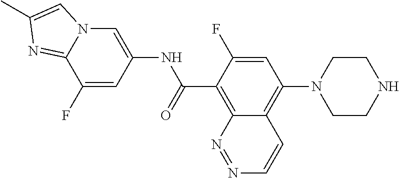 [SMILES: Cc1cn2cc(NC(=O)c3c(F)cc(N4CCNCC4)c4ccnnc34)cc(F)c2n1] |
| 351 | 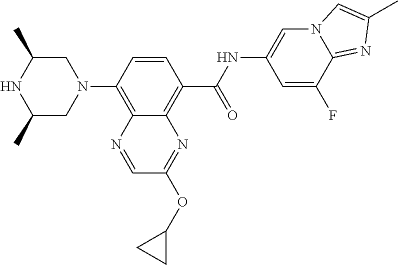 [SMILES: Cc1cn2cc(NC(=O)c3ccc(N4C[C@@H](C)N[C@@H](C)C4)c4ncc(OC5CC5)nc34)cc(F)c2n1] |
| 352 | 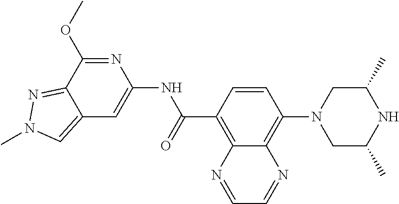 |
| 353 | 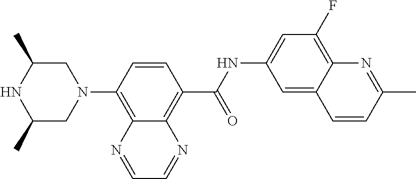 [SMILES: Cc1ccc2cc(NC(=O)c3ccc(N4C[C@@H](C)N[C@@H](C)C4)c4nccnc34)cc(F)c2n1] |
| 354 | 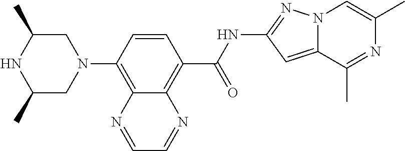 [SMILES: Cc1cn2nc(NC(=O)c3ccc(N4C[C@@H](C)N[C@@H](C)C4)c4nccnc34)cc2c(C)n1] |
| 355 | 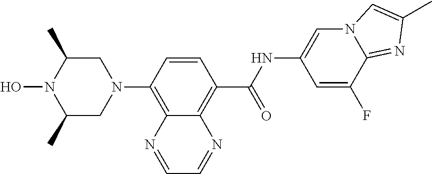 [SMILES: Cc1cn2cc(NC(=O)c3ccc(N4C[C@@H](C)N(O)[C@@H](C)C4)c4nccnc34)cc(F)c2n1] |
| 356 | 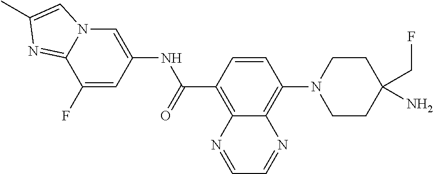 [SMILES: Cc1cn2cc(NC(=O)c3ccc(N4CCC(N)(CF)CC4)c4nccnc34)cc(F)c2n1] |
| 357 | 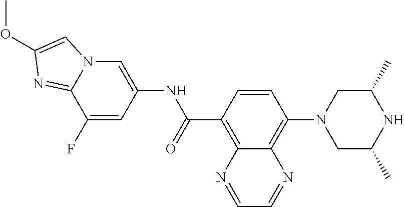 [SMILES: COc1cn2cc(NC(=O)c3ccc(N4C[C@@H](C)N[C@@H](C)C4)c4nccnc34)cc(F)c2n1] |
| 358 | 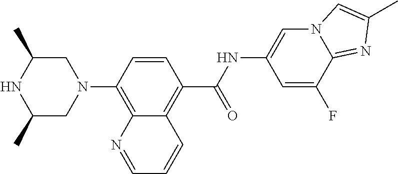 [SMILES: Cc1cn2cc(NC(=O)c3ccc(N4C[C@@H](C)N[C@@H](C)C4)c4ncccc34)cc(F)c2n1] |
| 359 | 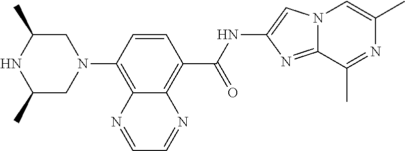 [SMILES: Cc1cn2cc(NC(=O)c3ccc(N4C[C@@H](C)N[C@@H](C)C4)c4nccnc34)nc2c(C)n1] |
| 360 | 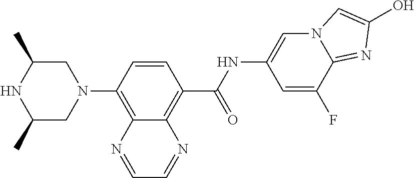 [SMILES: C[C@@H]1CN(c2ccc(C(=O)Nc3cc(F)c4nc(O)cn4c3)c3nccnc23)C[C@H](C)N1] |
| 361 | 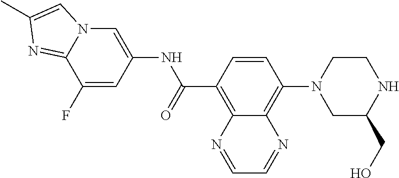 [SMILES: Cc1cn2cc(NC(=O)c3ccc(N4CCN[C@@H](CO)C4)c4nccnc34)cc(F)c2n1] |
| 362 | 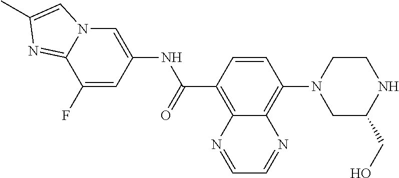 [SMILES: Cc1cn2cc(NC(=O)c3ccc(N4CCN[C@H](CO)C4)c4nccnc34)cc(F)c2n1] |
| 363 | 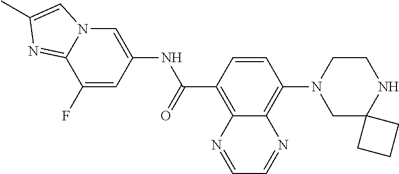 [SMILES: Cc1cn2cc(NC(=O)c3ccc(N4CCNC5(CCC5)C4)c4nccnc34)cc(F)c2n1] |
| 364 | 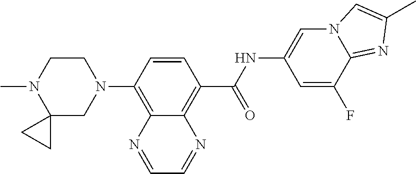 |
| 365 | 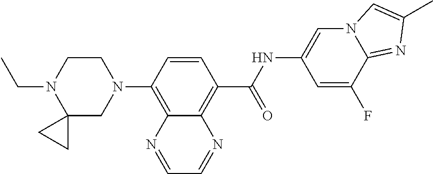 [SMILES: CCN1CCN(c2ccc(C(=O)Nc3cc(F)c4nc(C)cn4c3)c3nccnc23)CC12CC2] |
| 366 | 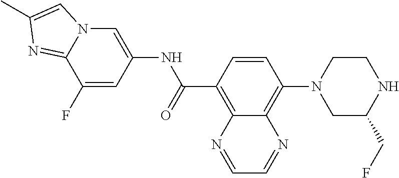 [SMILES: Cc1cn2cc(NC(=O)c3ccc(N4CCN[C@H](CF)C4)c4nccnc34)cc(F)c2n1] |
| 367 | 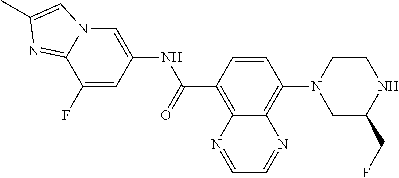 [SMILES: Cc1cn2cc(NC(=O)c3ccc(N4CCN[C@@H](CF)C4)c4nccnc34)cc(F)c2n1] |
| 368 | 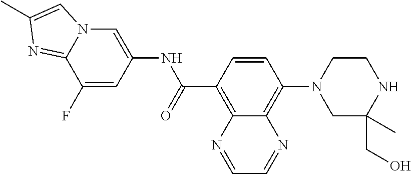 [SMILES: Cc1cn2cc(NC(=O)c3ccc(N4CCNC(C)(CO)C4)c4nccnc34)cc(F)c2n1] |
| 369 | 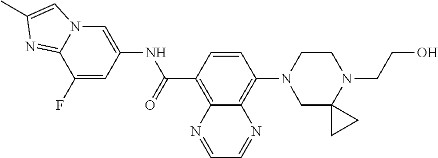 [SMILES: Cc1cn2cc(NC(=O)c3ccc(N4CCN(CCO)C5(CC5)C4)c4nccnc34)cc(F)c2n1] |
| 370 | 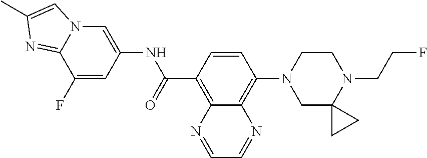 [SMILES: Cc1cn2cc(NC(=O)c3ccc(N4CCN(CCF)C5(CC5)C4)c4nccnc34)cc(F)c2n1] |
| 371 | 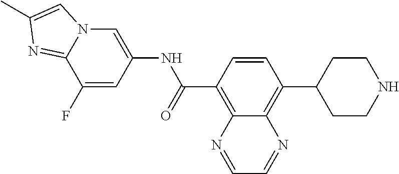 [SMILES: Cc1cn2cc(NC(=O)c3ccc(C4CCNCC4)c4nccnc34)cc(F)c2n1] |
| 372 | 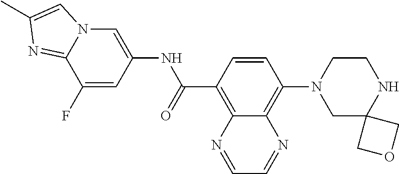 [SMILES: Cc1cn2cc(NC(=O)c3ccc(N4CCNC5(COC5)C4)c4nccnc34)cc(F)c2n1] |
| 373 | 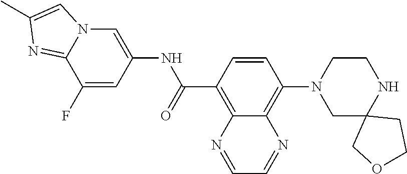 [SMILES: Cc1cn2cc(NC(=O)c3ccc(N4CCNC5(CCOC5)C4)c4nccnc34)cc(F)c2n1] |
| 374 | 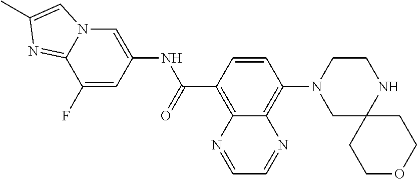 [SMILES: Cc1cn2cc(NC(=O)c3ccc(N4CCNC5(CCOCC5)C4)c4nccnc34)cc(F)c2n1] |
| 375 | 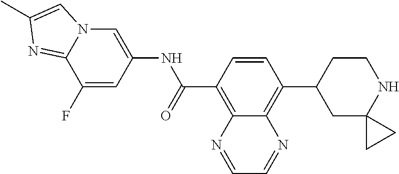 [SMILES: Cc1cn2cc(NC(=O)c3ccc(C4CCNC5(CC5)C4)c4nccnc34)cc(F)c2n1] |
| 376 | 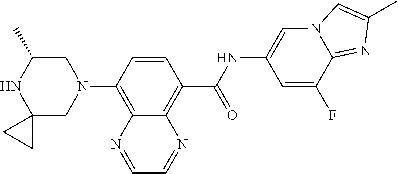 [SMILES: Cc1cn2cc(NC(=O)c3ccc(N4C[C@@H](C)NC5(CC5)C4)c4nccnc34)cc(F)c2n1] |
| 377 | 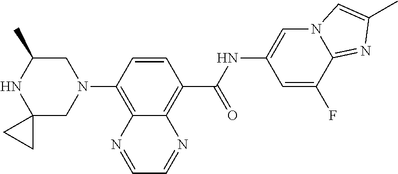 [SMILES: Cc1cn2cc(NC(=O)c3ccc(N4C[C@H](C)NC5(CC5)C4)c4nccnc34)cc(F)c2n1] |
| 378 | 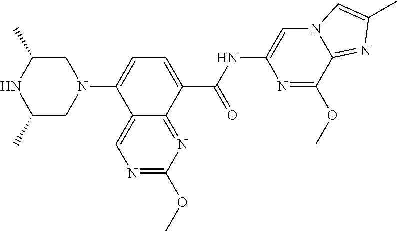 [SMILES: COc1ncc2c(N3C[C@@H](C)N[C@@H](C)C3)ccc(C(=O)Nc3cn4cc(C)nc4c(OC)n3)c2n1] |
| 379 | 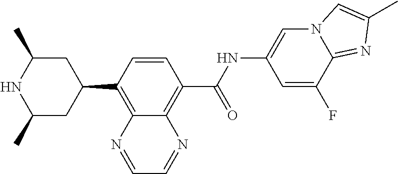 [SMILES: Cc1cn2cc(NC(=O)c3ccc([C@H]4C[C@@H](C)N[C@@H](C)C4)c4nccnc34)cc(F)c2n1] |
| 380 | 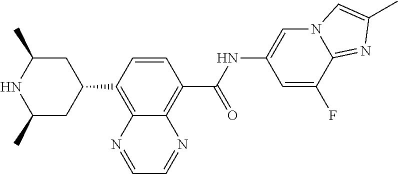 [SMILES: Cc1cn2cc(NC(=O)c3ccc([C@@H]4C[C@@H](C)N[C@@H](C)C4)c4nccnc34)cc(F)c2n1] |
| 381 | 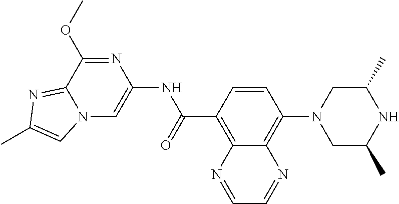 |
| 382 | 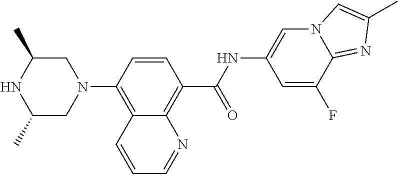 [SMILES: Cc1cn2cc(NC(=O)c3ccc(N4C[C@H](C)N[C@@H](C)C4)c4cccnc34)cc(F)c2n1] |
| 383 | 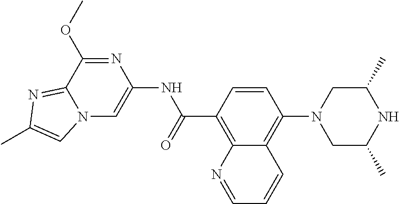 |
| 384 | 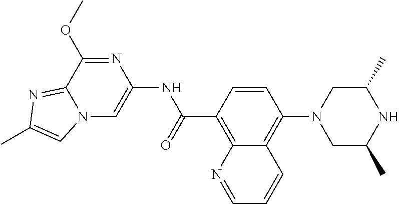 |
| 385 | 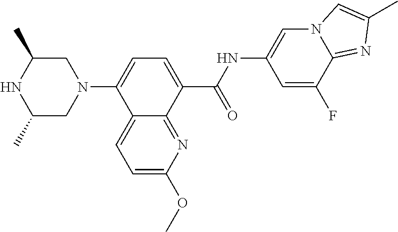 [SMILES: COc1ccc2c(N3C[C@H](C)N[C@@H](C)C3)ccc(C(=O)Nc3cc(F)c4nc(C)cn4c3)c2n1] |
| 386 | 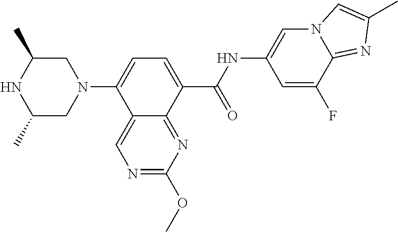 [SMILES: COc1ncc2c(N3C[C@H](C)N[C@@H](C)C3)ccc(C(=O)Nc3cc(F)c4nc(C)cn4c3)c2n1] |
| 387 | 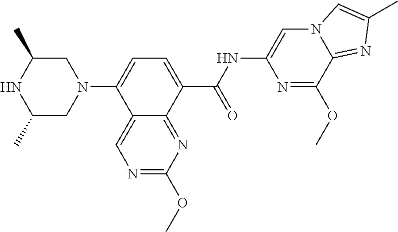 [SMILES: COc1ncc2c(N3C[C@H](C)N[C@@H](C)C3)ccc(C(=O)Nc3cn4cc(C)nc4c(OC)n3)c2n1] |
| 388 | 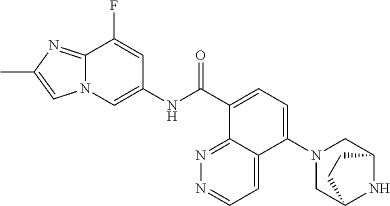 [SMILES: Cc1cn2cc(NC(=O)c3ccc(N4C[C@H]5CC[C@@H](C4)N5)c4ccnnc34)cc(F)c2n1] |
| 389 | 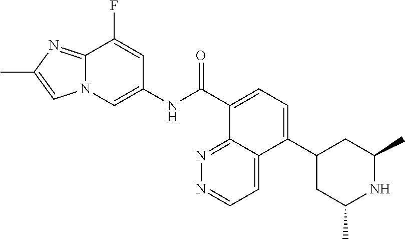 |
| 390 | 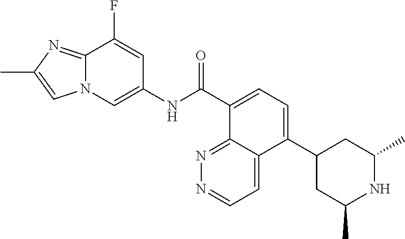 |
| 391 | 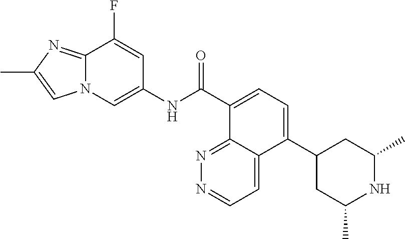 |
| 392 | 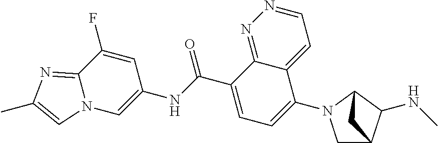 |
| 393 | 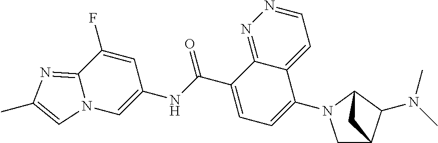 |
| 394 | 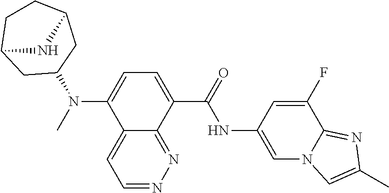 |
| 395 | 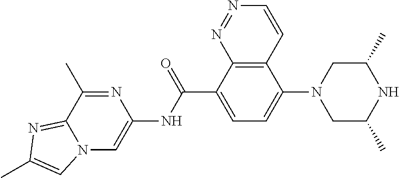 |
| 396 | 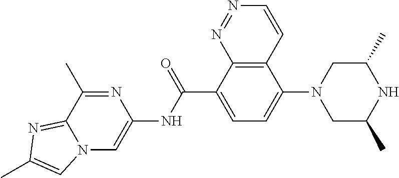 |
| 397 | 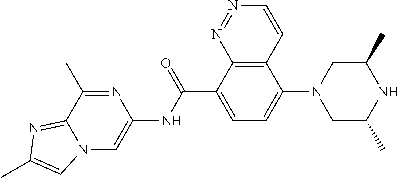 |
| 398 | 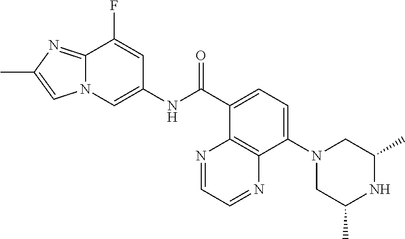 |
| 399 | 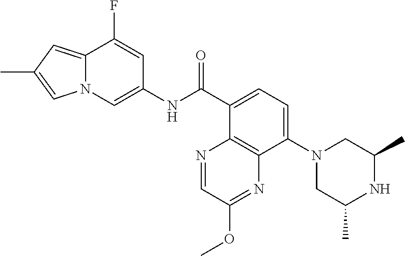 |
| 400 | 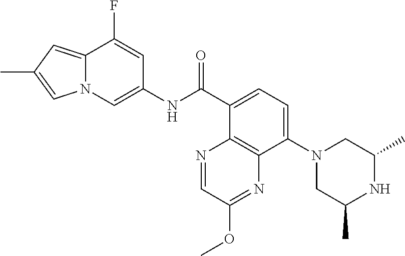 |
| 401 | 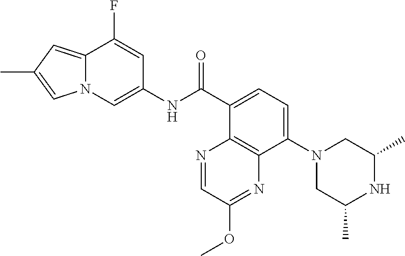 |
| 402 | 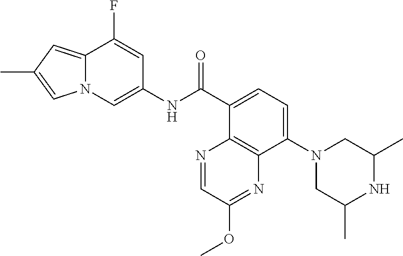 |
| 403 | 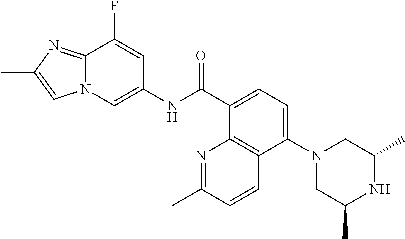 |
| 404 | 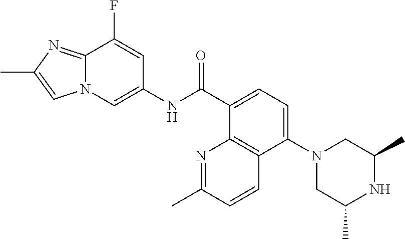 |
| 405 | 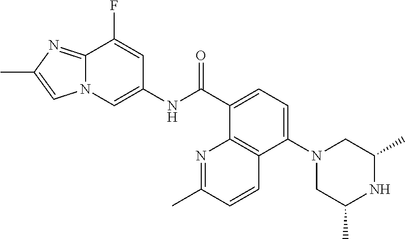 |
| 406 | 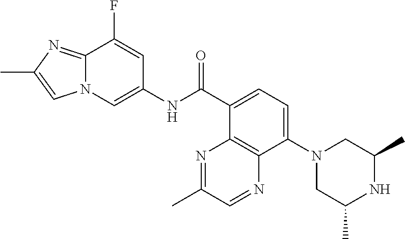 |
| 407 | 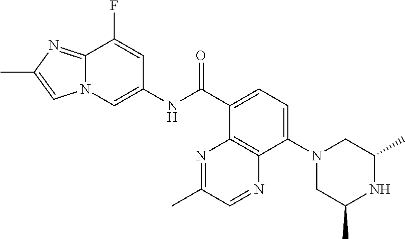 |
| 408 | 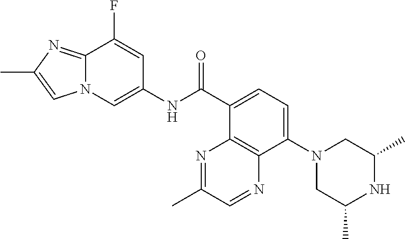 |
| 409 | 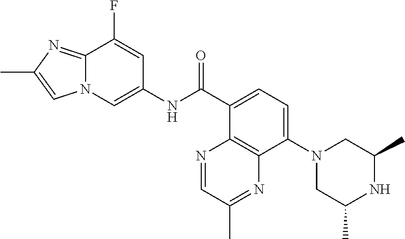 |
| 410 | 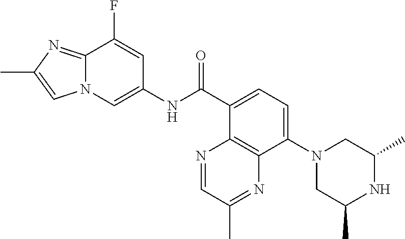 |
| 411 | 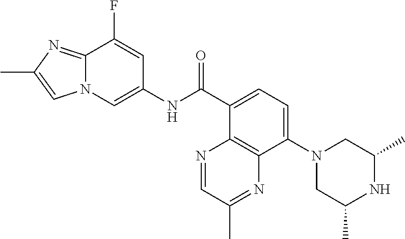 |
| 412 | 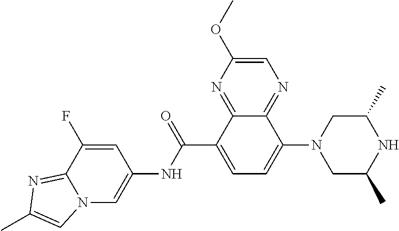 |
| 413 | 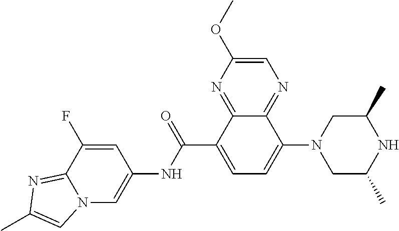 |
| 414 | 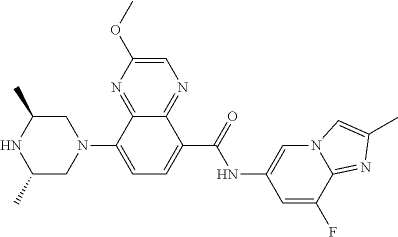 [SMILES: COc1cnc2c(C(=O)Nc3cc(F)c4nc(C)cn4c3)ccc(N3C[C@H](C)N[C@@H](C)C3)c2n1] |
| 415 | 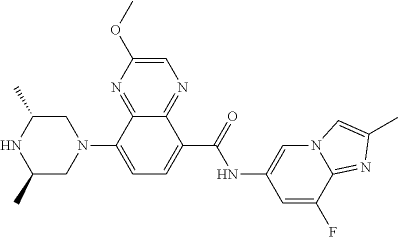 [SMILES: COc1cnc2c(C(=O)Nc3cc(F)c4nc(C)cn4c3)ccc(N3C[C@@H](C)N[C@H](C)C3)c2n1] |
| 416 | 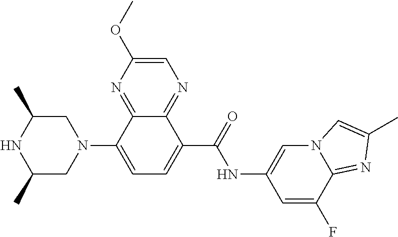 [SMILES: COc1cnc2c(C(=O)Nc3cc(F)c4nc(C)cn4c3)ccc(N3C[C@@H](C)N[C@@H](C)C3)c2n1] |
| 417 | 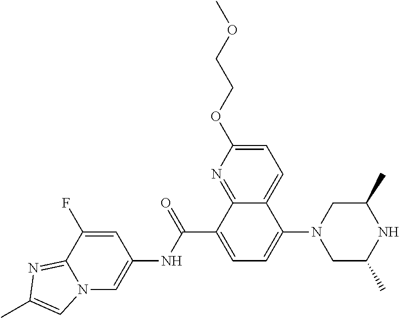 |
| 418 | 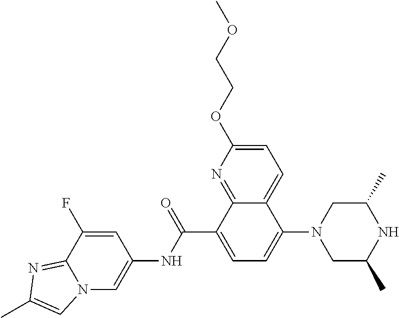 |
| 419 | 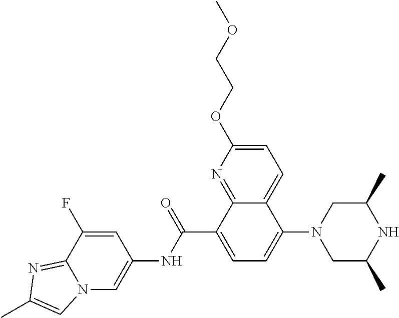 |
| 420 | 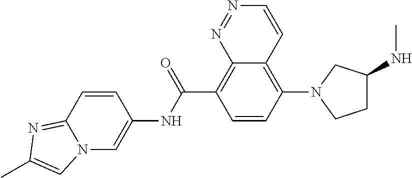 |
| 421 | 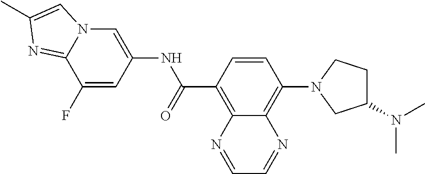 |
| 422 | 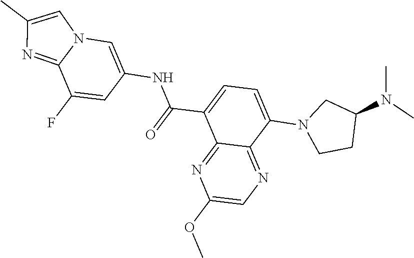 |
| 423 | 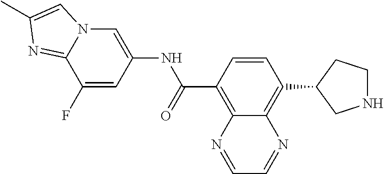 [SMILES: Cc1cn2cc(NC(=O)c3ccc([C@@H]4CCNC4)c4nccnc34)cc(F)c2n1] |
| 424 | 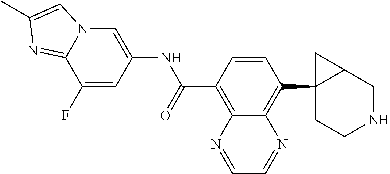 [SMILES: Cc1cn2cc(NC(=O)c3ccc([C@]45CCNCC4C5)c4nccnc34)cc(F)c2n1] |
| 425 | 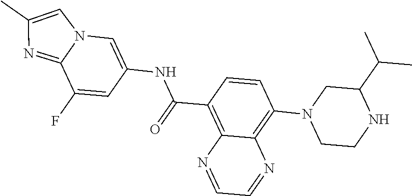 [SMILES: Cc1cn2cc(NC(=O)c3ccc(N4CCNC(C(C)C)C4)c4nccnc34)cc(F)c2n1] |
| 426 | 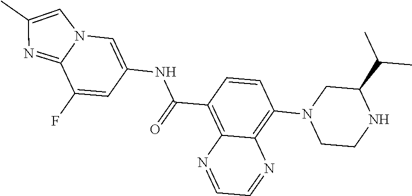 [SMILES: Cc1cn2cc(NC(=O)c3ccc(N4CCN[C@H](C(C)C)C4)c4nccnc34)cc(F)c2n1] |
| 427 | 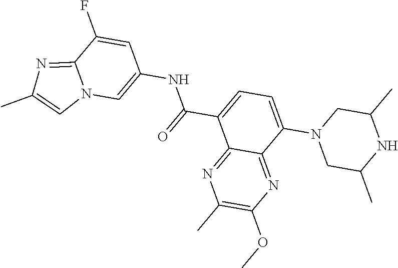 |
| 428 | 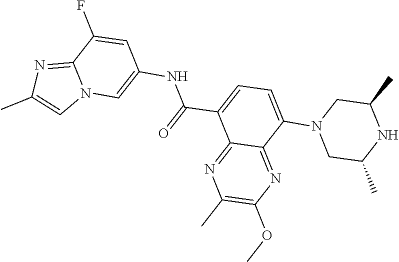 |
| 429 | 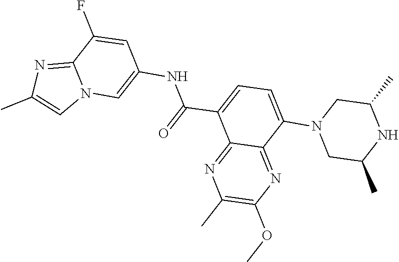 |
| 430 | 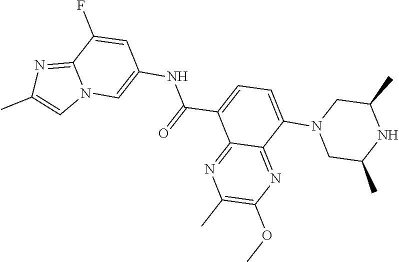 |
| 431 | 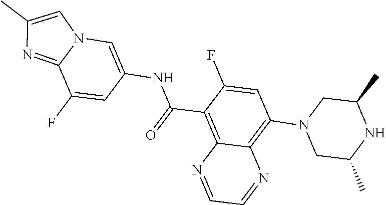 |
| 432 | 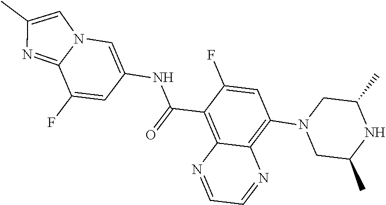 |
| 433 | 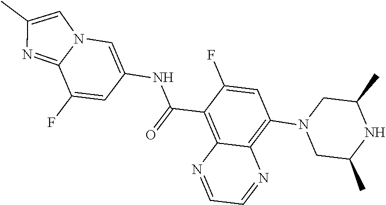 |
| 434 | 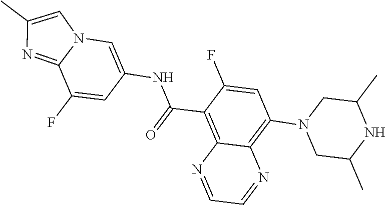 |
| 435 | 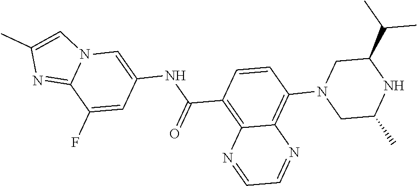 [SMILES: Cc1cn2cc(NC(=O)c3ccc(N4C[C@@H](C)N[C@H](C(C)C)C4)c4nccnc34)cc(F)c2n1] |
| 436 | 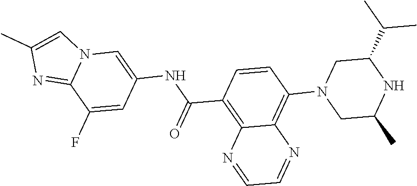 [SMILES: Cc1cn2cc(NC(=O)c3ccc(N4C[C@H](C)N[C@@H](C(C)C)C4)c4nccnc34)cc(F)c2n1] |
| 437 | 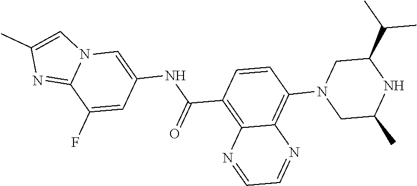 [SMILES: Cc1cn2cc(NC(=O)c3ccc(N4C[C@@H](C(C)C)N[C@@H](C)C4)c4nccnc34)cc(F)c2n1] |
| 438 | 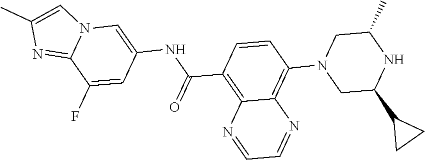 [SMILES: Cc1cn2cc(NC(=O)c3ccc(N4C[C@H](C)N[C@@H](C5CC5)C4)c4nccnc34)cc(F)c2n1] |
| 439 | 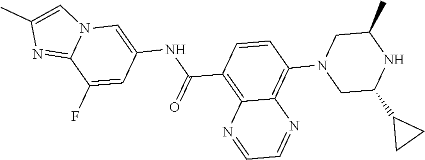 [SMILES: Cc1cn2cc(NC(=O)c3ccc(N4C[C@@H](C)N[C@H](C5CC5)C4)c4nccnc34)cc(F)c2n1] |
| 440 | 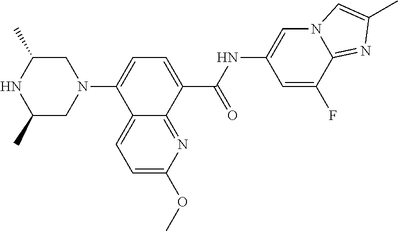 [SMILES: COc1ccc2c(N3C[C@@H](C)N[C@H](C)C3)ccc(C(=O)Nc3cc(F)c4nc(C)cn4c3)c2n1] |
| 441 | 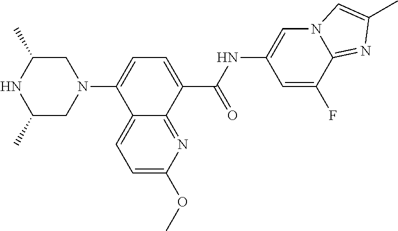 [SMILES: COc1ccc2c(N3C[C@@H](C)N[C@@H](C)C3)ccc(C(=O)Nc3cc(F)c4nc(C)cn4c3)c2n1] |
| 442 | 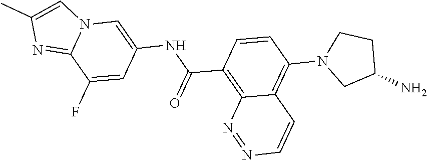 [SMILES: Cc1cn2cc(NC(=O)c3ccc(N4CC[C@H](N)C4)c4ccnnc34)cc(F)c2n1] |
| 443 | 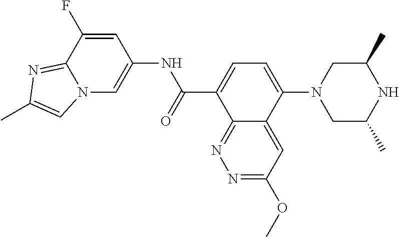 |
| 444 | 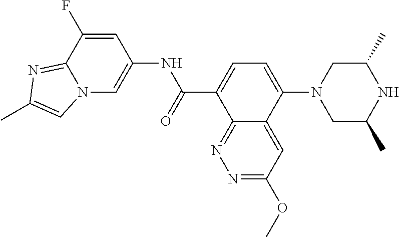 |
| 445 | 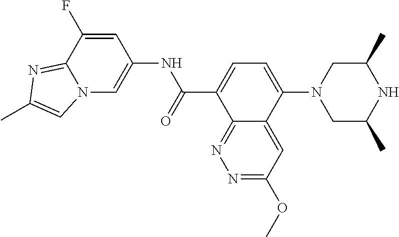 |
| 446 | 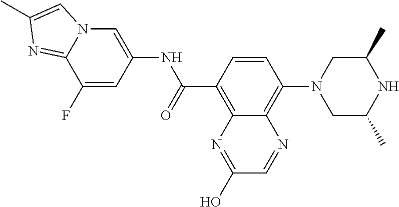 |
| 447 | 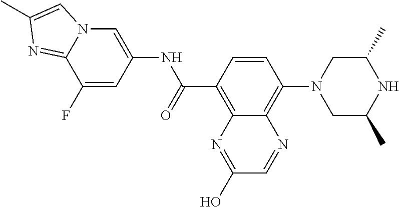 |
| 448 | 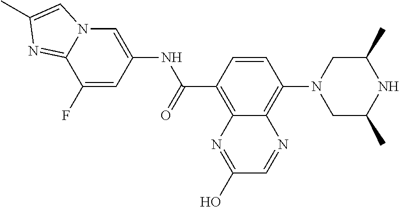 |
| 449 | 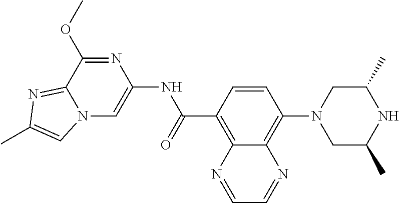 |
| 450 | 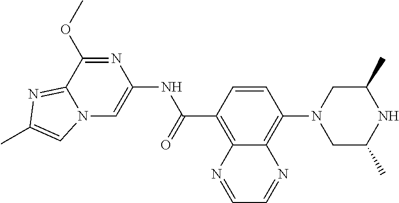 |
| 451 | 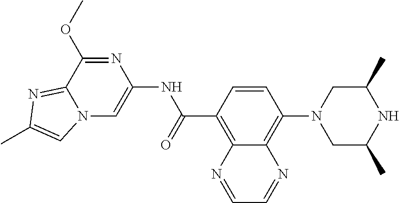 |
| 452 | 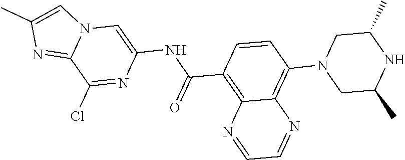 |
| 453 | 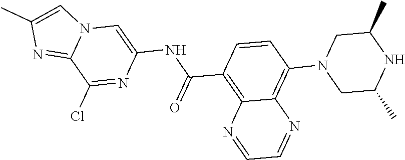 |
| 454 | 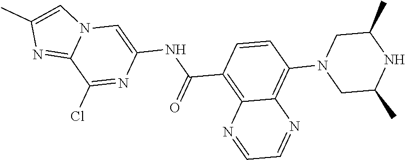 |
| 455 | 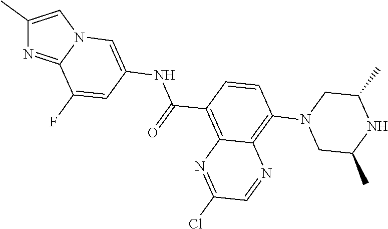 |
| 456 | 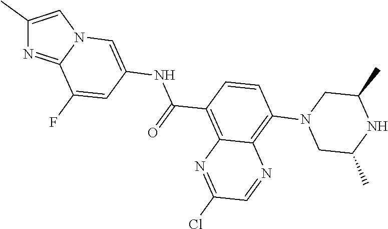 |
| 457 | 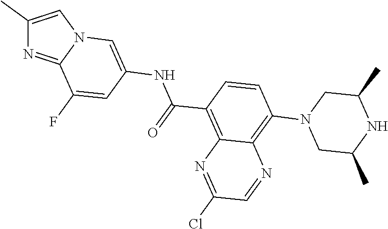 |
| 458 | 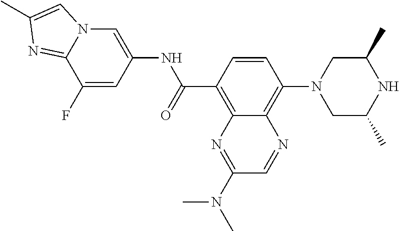 |
| 459 | 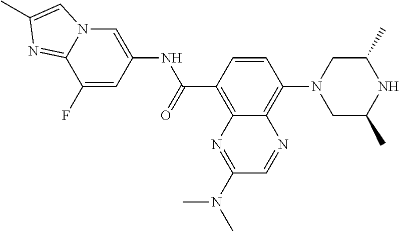 |
| 460 | 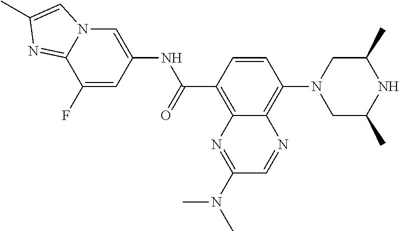 |
| 461 | 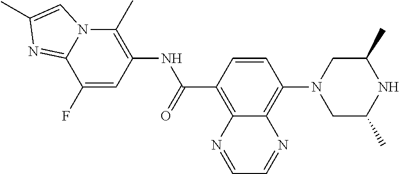 |
| 462 | 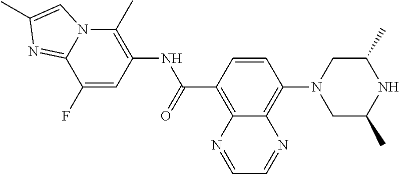 |
| 463 | 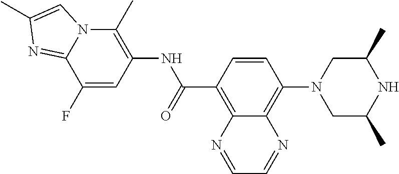 |
| 464 | 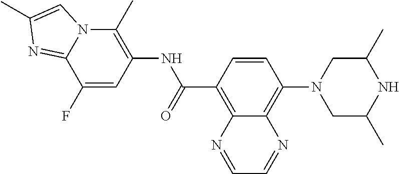 |
| 465 | 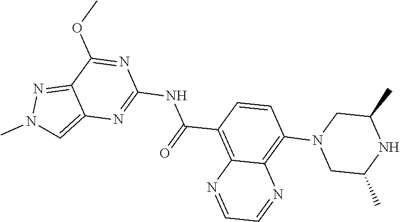 |
| 466 | 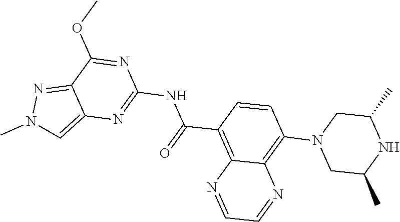 |
| 467 | 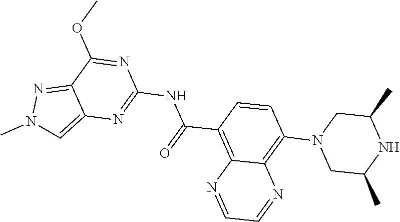 |
| 468 | 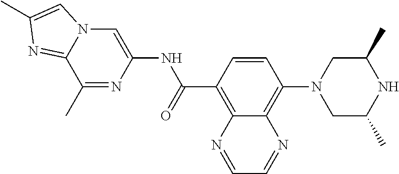 |
| 469 | 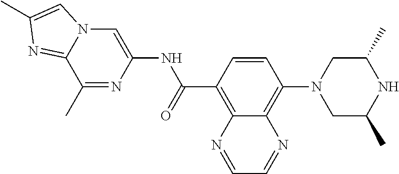 |
| 470 | 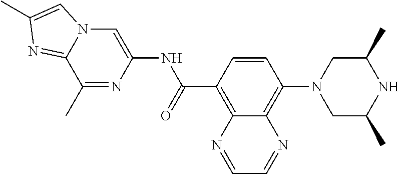 |
| 471 | 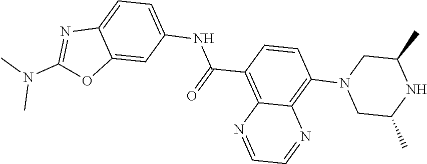 |
| 472 | 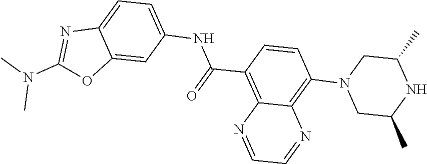 |
| 473 | 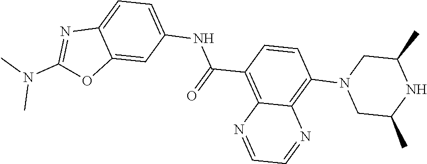 |
| 474 | 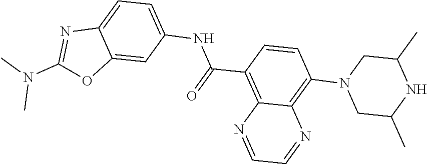 |
| 475 | 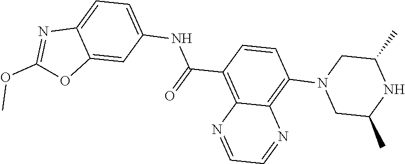 |
| 476 | 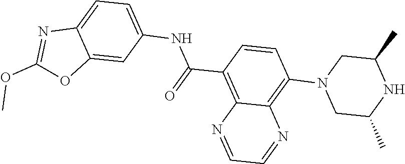 |
| 477 | 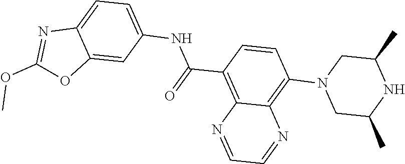 |
| 478 | 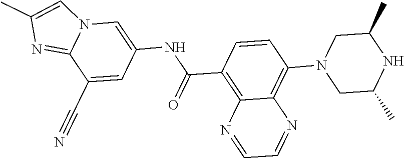 |
| 479 | 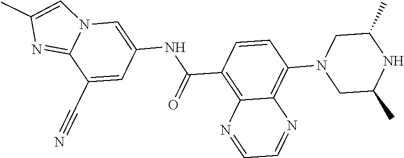 |
| 480 | 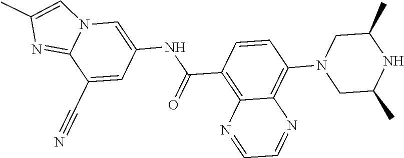 |
| 481 | 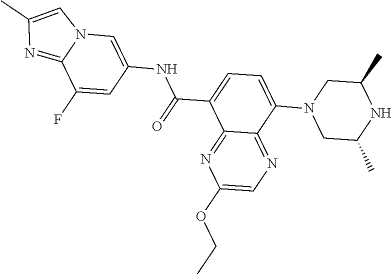 |
| 482 | 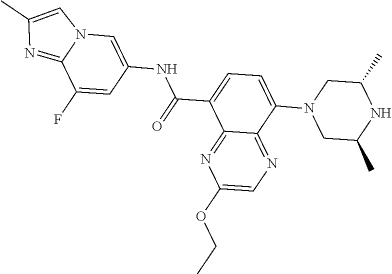 |
| 483 | 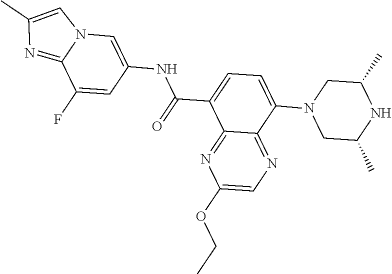 |
| 484 | 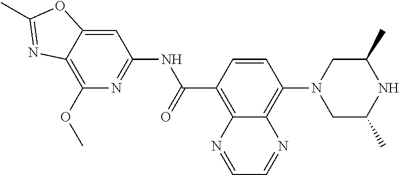 |
| 485 | 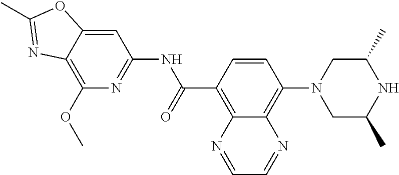 |
| 486 | 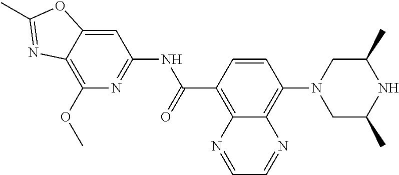 |
| 487 | 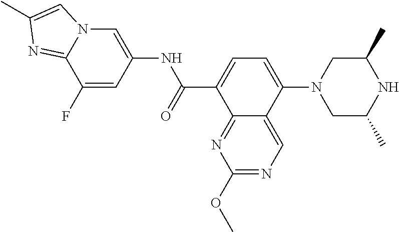 |
| 488 | 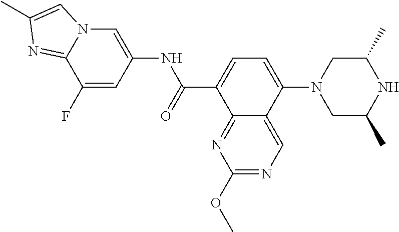 |
| 489 | 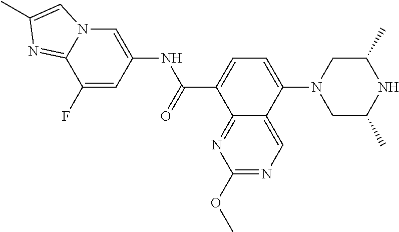 |
| 490 | 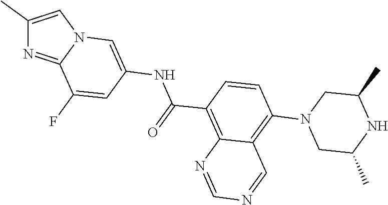 |
| 491 | 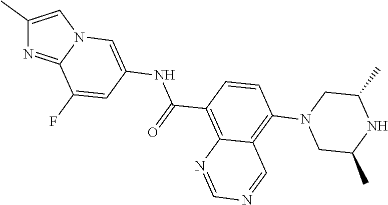 |
| 492 | 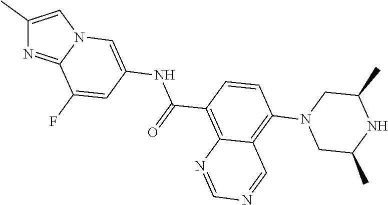 |
| 493 | 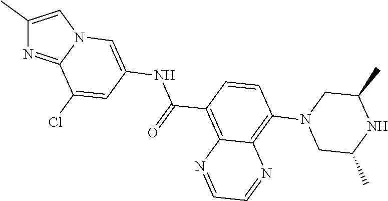 |
| 494 | 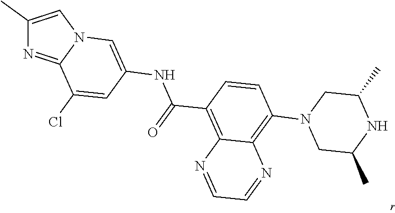 |
| 495 | 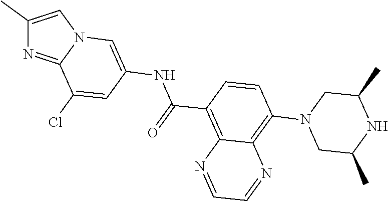 |
| 496 | 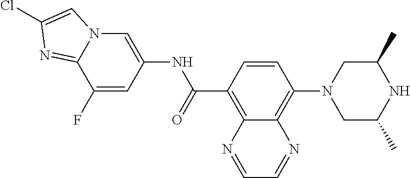 [SMILES: C[C@@H]1CN(c2ccc(C(=O)Nc3cc(F)c4nc(Cl)cn4c3)c3nccnc23)C[C@@H](C)N1] |
| 497 | 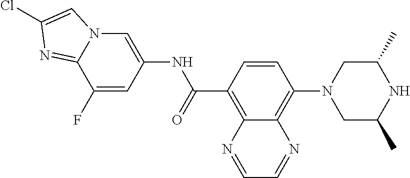 [SMILES: C[C@H]1CN(c2ccc(C(=O)Nc3cc(F)c4nc(Cl)cn4c3)c3nccnc23)C[C@H](C)N1] |
| 498 | 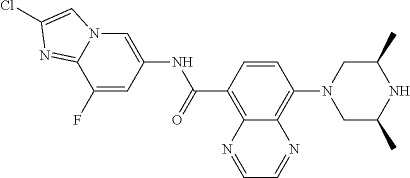 [SMILES: C[C@@H]1CN(c2ccc(C(=O)Nc3cc(F)c4nc(Cl)cn4c3)c3nccnc23)C[C@H](C)N1] |
| 499 | 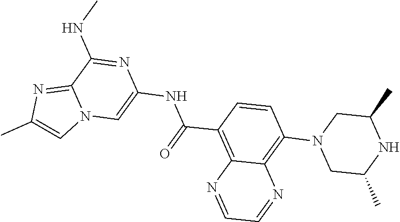 |
| 500 | 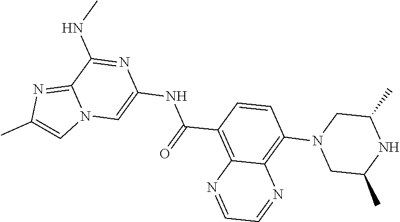 |
| 501 | 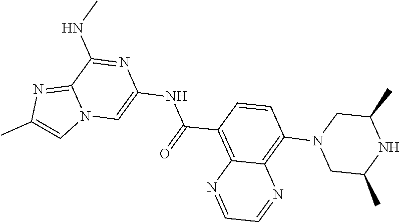 |
| 502 | 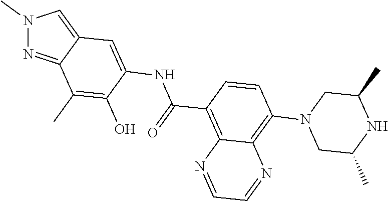 |
| 503 | 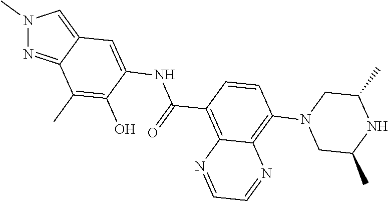 |
| 504 | 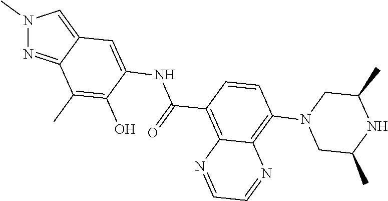 |
| 505 | 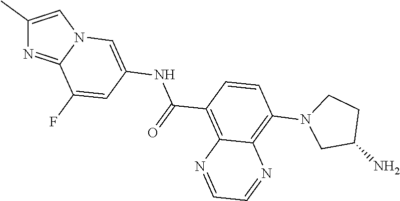 [SMILES: Cc1cn2cc(NC(=O)c3ccc(N4CC[C@H](N)C4)c4nccnc34)cc(F)c2n1] |
| 506 | 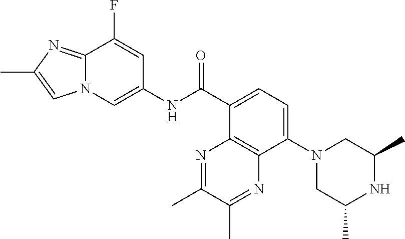 |
| 507 | 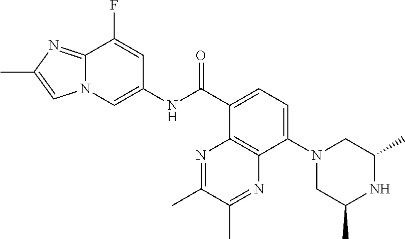 |
| 508 | 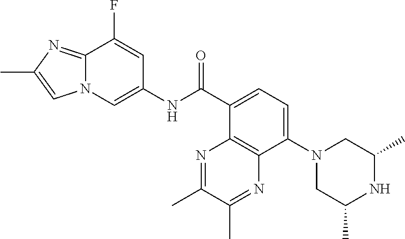 |
| 509 | 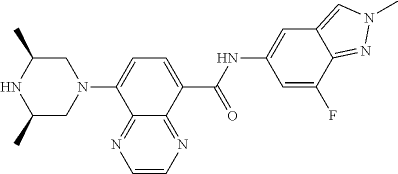 [SMILES: C[C@@H]1CN(c2ccc(C(=O)Nc3cc(F)c4nn(C)cc4c3)c3nccnc23)C[C@H](C)N1] |
| 510 | 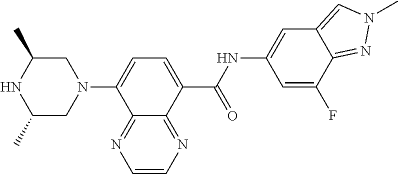 [SMILES: C[C@H]1CN(c2ccc(C(=O)Nc3cc(F)c4nn(C)cc4c3)c3nccnc23)C[C@H](C)N1] |
| 511 | 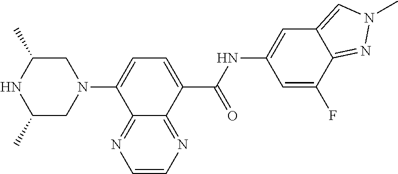 [SMILES: C[C@@H]1CN(c2ccc(C(=O)Nc3cc(F)c4nn(C)cc4c3)c3nccnc23)C[C@H](C)N1] |
| 512 | 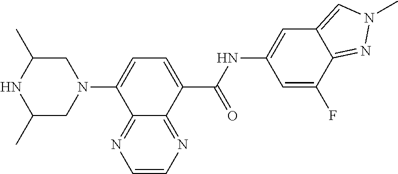 [SMILES: CC1CN(c2ccc(C(=O)Nc3cc(F)c4nn(C)cc4c3)c3nccnc23)CC(C)N1] |
| 513 | 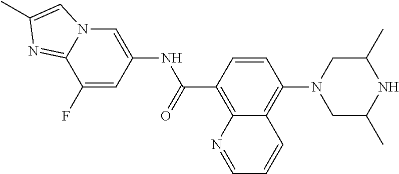 |
| 514 | 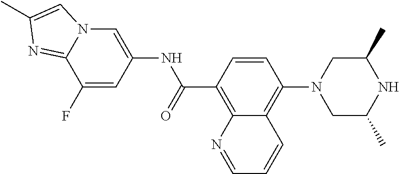 |
| 515 | 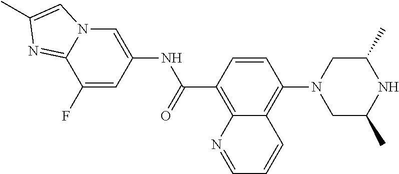 |
| 516 | 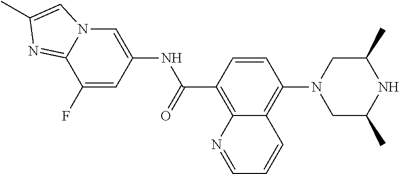 |
| 517 | 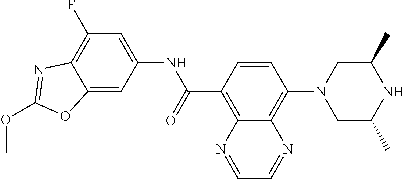 [SMILES: COc1nc2c(F)cc(NC(=O)c3ccc(N4C[C@@H](C)N[C@H](C)C4)c4nccnc34)cc2o1] |
| 518 | 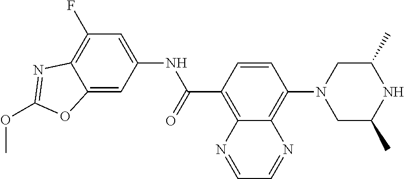 [SMILES: COc1nc2c(F)cc(NC(=O)c3ccc(N4C[C@H](C)N[C@@H](C)C4)c4nccnc34)cc2o1] |
| 519 | 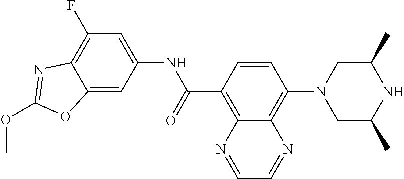 [SMILES: COc1nc2c(F)cc(NC(=O)c3ccc(N4C[C@@H](C)N[C@@H](C)C4)c4nccnc34)cc2o1] |
| 520 | 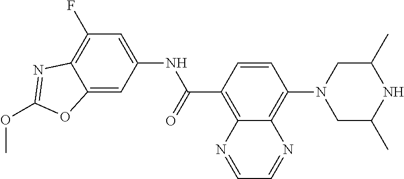 [SMILES: COc1nc2c(F)cc(NC(=O)c3ccc(N4CC(C)NC(C)C4)c4nccnc34)cc2o1] |
| 521 | 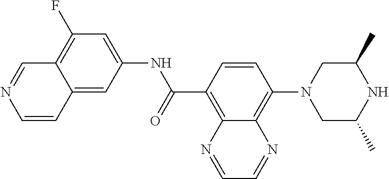 [SMILES: C[C@@H]1CN(c2ccc(C(=O)Nc3cc(F)c4cnccc4c3)c3nccnc23)C[C@@H](C)N1] |
| 522 | 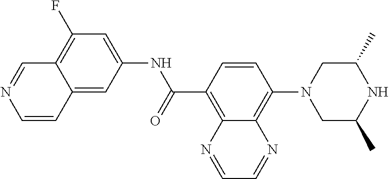 [SMILES: C[C@H]1CN(c2ccc(C(=O)Nc3cc(F)c4cnccc4c3)c3nccnc23)C[C@H](C)N1] |
| 523 | 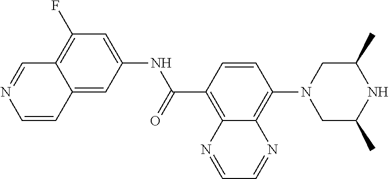 [SMILES: C[C@@H]1CN(c2ccc(C(=O)Nc3cc(F)c4cnccc4c3)c3nccnc23)C[C@H](C)N1] |
| 524 | 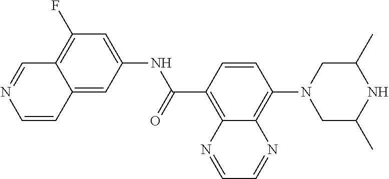 [SMILES: CC1CN(c2ccc(C(=O)Nc3cc(F)c4cnccc4c3)c3nccnc23)CC(C)N1] |
| 525 | 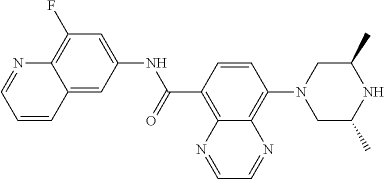 [SMILES: C[C@@H]1CN(c2ccc(C(=O)Nc3cc(F)c4ncccc4c3)c3nccnc23)C[C@@H](C)N1] |
| 526 | 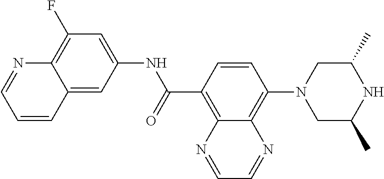 [SMILES: C[C@H]1CN(c2ccc(C(=O)Nc3cc(F)c4ncccc4c3)c3nccnc23)C[C@H](C)N1] |
| 527 | 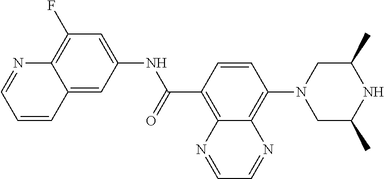 [SMILES: C[C@@H]1CN(c2ccc(C(=O)Nc3cc(F)c4ncccc4c3)c3nccnc23)C[C@H](C)N1] |
| 529 | 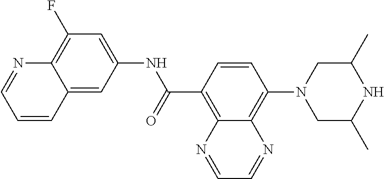 [SMILES: CC1CN(c2ccc(C(=O)Nc3cc(F)c4ncccc4c3)c3nccnc23)CC(C)N1] |

embodiments, a compound of formula (is selected from Compounds 161-184. In some embodiments, a compound of Formula (I) is a compound other than Compounds 100-160. In some embodiments, a compound of Formula (I) is selected from Compound 101, 103, 112, 128, 130, 137, 146, 147, 149, 151, 153, 157, 158, 159, 161, 162, 165, 169, 171, 172, or 173. In some embodiments, a compound of Formula (I-g) is selected from Compound 101, 103, 112, 128, 130, 137, 146, 147, 149, 151, 153, 157, 158, 159, 161, 162, 165, 169, 171, 172, or 173.

In some embodiments, a compound of Formula (I) is: a) Compound 161; b) Compound 162; c) Compound 163; d) Compound 164; e) Compound 165; f) Compound 166; g) Compound 167; h) Compound 168; i) Compound 169; j) Compound 170; k) Compound 171; 1) Compound 172; m) Compound 173; n) Compound 174; p) Compound 176; q) Compound 177; r) Compound 178; s) Compound 179; t) Compound 180; u) Compound 181; v) Compound 182; w) Compound 183; or x) Compound 184. In some embodiments, for Formula (I), A is monocyclic heterocyclyl (e.g., piperazinyl); B is bicyclic heteroaryl (e.g., 2,8-dimethylimidazo[1,2-b]pyridazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 100, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 101, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 103, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 104, or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 105, or a pharmaceutically acceptable salt, solvate, hydrate, tautomer, or stereoisomer thereof.

In some embodiments, for Formula (I), A is monocyclic heterocyclyl (e.g., 2,2-dimethylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 106, or a pharmaceutically acceptable salt, solvate, hydrate, tautomer, or stereoisomer thereof.

In some embodiments, for Formula (I), A is bicyclic heterocyclyl (e.g., octahydropyrrolo[1,2-a]pyrazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 107, 153, 154, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 108, or a pharmaceutically acceptable salt, solvate, hydrate, tautomer, or stereoisomer thereof.

In some embodiments, for Formula (I), A is monocyclic heterocyclyl (e.g., 3-(tert-butylamino)pyrrolidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 109, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); X is C(R^{3a}) (e.g., CH); W, Y, and Z are each N; and y is 1. In some embodiments, the compound of Formula (I), (I-a), and (I-e) is Compound 110, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); X and Y are each independently C(R^{3a}) (e.g., CH); W and Z are N; and y is 1. In some embodiments, the compound of Formula (I), (I-a), and (I-e) is Compound 111, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and Y are each independently C(R^{3a}) (e.g., CH); X and Z are N; and y is 1. In some embodiments, the compound of Formula (I), (I-a), (I-b), (I-e), (I-f), and (I-g) is Compound 112, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-b]pyridazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 113, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is absent; W is C(R^{3a}) (e.g., CH); X is C(R^{3a}) (e.g., CH); Y is C(R^{3a}) (e.g., C(OCH₃)); Z is N; and y is 1. In some embodiments, the compound of Formula (I), (I-a), and (I-b) is Compound 114, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methyl-2H-indazolyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 115, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methyl-2H-indazolyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 116, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]oxazolyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 117, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4-fluoro-2-methylbenzo[d]thiazolyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 118, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 119, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methylimidazo[1,2-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 120, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6,8-dimethylimidazo[1,2-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 121, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6,8-dimethyl-[1,2,4]triazolo[1,5-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 122, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 4,6-dimethylpyrazolo[1,5-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 123, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-chloro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 124, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 125, or a pharmaceutically acceptable salt, solvate, hydrate, tautomer, or stereoisomer thereof.

In some embodiments, for Formula (I), A is monocyclic heterocyclyl (e.g., piperazinyl); B is monocyclic heteroaryl (e.g., pyrazyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 126, or a pharmaceutically acceptable salt, solvate, hydrate, tautomer, or stereoisomer thereof.

In some embodiments, for Formula (I), A is monocyclic heterocyclyl (e.g., 1-methylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 127, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 128, 146, 147 or a pharmaceutically acceptable salt, solvate, hydrate, tautomer, or stereoisomer thereof.

In some embodiments, for Formula (I), A is monocyclic heterocyclyl (e.g., 1,2-dimethylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 129, 149, 150, or a pharmaceutically acceptable salt, solvate, hydrate, tautomer, or stereoisomer thereof.

In some embodiments, for Formula (I), A is monocyclic heterocyclyl (e.g., 2-ethylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 130, 151, 152, or a pharmaceutically acceptable salt, solvate, hydrate, tautomer, or stereoisomer thereof.

In some embodiments, for Formula (I), A is bicyclic heterocyclyl (e.g., 2,6-diazaspiro[3.3]heptanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 131, or a pharmaceutically acceptable salt, solvate, hydrate, tautomer, or stereoisomer thereof.

In some embodiments, for Formula (I), A is bicyclic heterocyclyl (e.g., 2-methyl-2,6-diazaspiro[3.3]heptanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 132, or a pharmaceutically acceptable salt, solvate, hydrate, tautomer, or stereoisomer thereof.

In some embodiments, for Formula (I), A is monocyclic heterocyclyl (e.g., 1,3′-bipyrrolidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 133, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 134, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ is —N(R⁴)—(e.g., —N(CH₃)—); L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 135, or a pharmaceutically acceptable salt, solvate, hydrate, tautomer, or stereoisomer thereof.

In some embodiments, for Formula (I), A is monocyclic heterocyclyl (e.g., 2-methylpiperidinyl); B is bicyclic heteroaryl (e.g., 8-fluoro-2-methylimidazo[1,2-a]pyridinyl); L¹ is —N(R⁴)—(e.g., —N(CH₃)—); L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 136, or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 137, or a pharmaceutically acceptable salt, solvate, hydrate, tautomer, or stereoisomer thereof.

In some embodiments, for Formula (I), A is monocyclic heterocyclyl (e.g., 4-(N-tert-butyl)aminopiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 138, or a pharmaceutically acceptable salt, solvate, hydrate, tautomer, or stereoisomer thereof.

In some embodiments, for Formula (I), A is monocyclic heterocyclyl (e.g., 4-(N ethyl)aminopiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 139, or a pharmaceutically acceptable salt, solvate, hydrate, tautomer, or stereoisomer thereof.

In some embodiments, for Formula (I), A is monocyclic heterocyclyl (e.g., 1-methylpiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 140, or a pharmaceutically acceptable salt, solvate, hydrate, tautomer, or stereoisomer thereof.

In some embodiments, for Formula (I), A is monocyclic heterocyclyl (e.g., 1-ethylpiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 141, or a pharmaceutically acceptable salt, solvate, hydrate, tautomer, or stereoisomer thereof.

In some embodiments, for Formula (I), A is monocyclic heterocyclyl (e.g., 2,6-dimethylpiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 142, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 143, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ and L² are absent; W is C(R^{3a}) (e.g., CH); Y is C(R^{3a}) (e.g., CH); X is C(R^{3a}) (e.g., C(OH)); and Z is N; and y is 1. In some embodiments, the compound of Formula (I), (I-a), and (I-b) is Compound 144, or a pharmaceutically acceptable salt, solvate, hydrate, tautomer, or stereoisomer thereof.

In some embodiments, for Formula (I), A is bicyclic heterocyclyl (e.g., (1R,5S)-3,8-diazabicyclo[3.2.1]octanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 145, or a pharmaceutically acceptable salt, solvate, hydrate, tautomer, or stereoisomer thereof.

In some embodiments, for Formula (I), A is bicyclic heterocyclyl (e.g., 5-methylamino-2-azabicyclo[2.1.1]hexanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 148, 160, or a pharmaceutically acceptable salt, solvate, hydrate, tautomer, or stereoisomer thereof.

In some embodiments, for Formula (I), A is monocyclic heterocyclyl (e.g., (1R,4S)-5-dimethylamino-2-azabicyclo[2.1.1]hexanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 155, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is bicyclic heteroaryl (e.g., 8-fluoro-2-methylimidazo[1,2-a]pyridinyl); L¹ is —N(R⁴)—(e.g., —N(CH₃)—); L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 156, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); X and Y are each independently C(R^{3a}) (e.g., CH); W and Z are N; and y is 1. In some embodiments, the compound of Formula (I), (I-a), and (I-e) is Compound 157 or a pharmaceutically acceptable salt, solvate, hydrate, tautomer, or stereoisomer thereof.

In some embodiments, for Formula (I), A is monocyclic heterocyclyl (e.g., 4-(N,N-dimethyl)aminopiperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); X and Y are each independently C(R^{3a}) (e.g., CH); W and Z are N; and y is 1. In some embodiments, the compound of Formula (I), (I-a), and (I-e) is Compound 158 or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., C(CH₃)); Y and Z are N; and y is 1. In some embodiments, the compound of Formula (I), (I-a), (I-b), (I-e), (I-f), and (I-g) is Compound 159 or a pharmaceutically acceptable salt, solvate, hydrate, tautomer, or stereoisomer thereof.

In some embodiments, for Formula (I), A is monocyclic heterocyclyl (e.g., (2S,6R)-2,6-dimethyl-4-piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 161, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., CH); Y is C(R^{3a}) (e.g., C(CH₃)); Z is N; and y is 1. In some embodiments, the compound of Formula (I), (I-a), (I-b), (I-e), (I-f), and (I-g) is Compound 162, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 1-(2-methylimidazo[1,2-a]146yridine-8-yl)ethan-1-I); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 163, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,8-dimethylimidazo[1,2-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 164, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., CH); Y is C(R^{3a}) (e.g., CH); Z is N; and y is 1. In some embodiments, the compound of Formula (I), (I-a), (I-b), (I-e), (I-f), and (I-g) is Compound 165, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is N; Y is C(R^{3a}) (e.g., CH); Z is C(R^{3a}) (e.g., CH); and y is 1. In some embodiments, the compound of Formula (I), (I-a), and (I-e) is Compound 166, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-cyano-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 167, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-ethyl-2-methylimidazo[1,2-b]pyridazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 168, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., CH); Y is C(R^{3a}) (e.g., C(OCH₃)); Z is N; and y is 1. In some embodiments, the compound of Formula (I), (I-a), (I-b), (I-e), (I-f), and (I-g) is Compound 169, or a pharmaceutically acceptable salt, solvate, hydrate, tautomer, or stereoisomer thereof.

In some embodiments, for Formula (I), A is bicyclic heterocyclic (e.g., 2,8-dimethylimidazo[1,2-b]pyridazinyl); B is monocyclic heterocyclyl (e.g., piperadinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 170, or a pharmaceutically acceptable salt, solvate, hydrate, tautomer, or stereoisomer thereof.

In some embodiments, for Formula (I), A is bicyclic heterocyclyl (e.g., 1,7-diazaspiro[3.5]nonanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., CH); Y is N; Z is N; and y is 1. In some embodiments, the compound of Formula (I), (I-a), (I-b), (I-e), (I-f), and (I-g) is Compound 171, or a pharmaceutically acceptable salt, solvate, hydrate, tautomer, or stereoisomer thereof.

In some embodiments, for Formula (I), A is monocyclic heterocyclyl (e.g., (2S,6R)-2,6-dimethyl-4-piperazinyl); B is bicyclic heteroaryl (e.g., 2,8-dimethylimidazo[1,2-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 172, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., CH); Y is C(R^{3a}) (e.g., C(OCH₂CH₂OCH₃)); Z is N; and y is 1. In some embodiments, the compound of Formula (I), (I-a), (I-b), (I-e), (I-f), and (I-g) is Compound 173, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-methoxy-2-methylimidazo[1,2-a]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 176, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2-methylimidazo[1,2-a]pyrazin-8(7H)—I); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 177, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 2,7-dimethyl-2H-pyrazolo[3,4-c]pyridinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 178, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-methoxy-2-methylimidazo[1,2-a]pyrazinyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 179, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is N; Y is C(R^{3a}) (e.g., C(OCH₂CH₂OCH₃)); Z is N; and y is 1. In some embodiments, the compound of Formula (I), (I-a), (I-b), (I-e), (I-f), and (I-g) is Compound 180, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 6-hydroxy-2,7-dimethyl-2H-indazolyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), and (I-f) is Compound 181, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 1-(2-methylimidazo[1,2-a]150yridine-8-yl)ethan-1-olyl); L¹ is absent; L² is —C(O)N(R⁴)—(e.g., —C(O)N(H)—); W and X are each independently C(R^{3a}) (e.g., CH); Y and Z are N; and y is 1. In some embodiments, the compound of Formula (I), (I-a), (I-b), (I-d), (I-e), (I-f), and (I-g) is Compound 182, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., C(OCH₃)); Y and Z are N; and y is 1. In some embodiments, the compound of Formula (I), (I-a), (I-b), (I-d), (I-e), (I-f), and (I-g) is Compound 183, or a pharmaceutically acceptable salt, solvate, hydrate, tautomer, or stereoisomer thereof.

In some embodiments, for Formula (I), A is monocyclic heterocyclyl (e.g., piperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is C(R^{3a}) (e.g., CH); X is C(R^{3a}) (e.g., C(CH₂CH₃)); Y and Z are N; and y is 1. In some embodiments, the compound of Formula (I), (I-a), (I-b), (I-d), (I-e), (I-f), and (I-g) is Compound 185, or a pharmaceutically acceptable salt, solvate, hydrate, tautomer, or stereoisomer thereof.

In some embodiments, for Formula (I), A is monocyclic heterocyclyl (e.g., 2,6-dimethylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 186, or a pharmaceutically acceptable salt, solvate, hydrate, tautomer, or stereoisomer thereof.

In some embodiments, for Formula (I), A is monocyclic heterocyclyl (e.g., 2-methylpiperazinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 187, 188, or a pharmaceutically acceptable salt, solvate, hydrate, tautomer, or stereoisomer thereof.

In some embodiments, for Formula (I), A is monocyclic heterocyclyl (e.g., 1,7-diazaspiro[3.5]nonanyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 189, or a pharmaceutically acceptable salt, solvate, hydrate, tautomer, or stereoisomer thereof.

In some embodiments, for Formula (I), A is monocyclic heterocyclyl (e.g., 3-(N-methylamino)pyrrolidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 190, 193, 194, or a pharmaceutically acceptable salt, solvate, hydrate, tautomer, or stereoisomer thereof.

In some embodiments, for Formula (I), A is monocyclic heterocyclyl (e.g., 4-(N-ethylamino)piperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 191, or a pharmaceutically acceptable salt, solvate, hydrate, tautomer, or stereoisomer thereof.

In some embodiments, for Formula (I), A is monocyclic heterocyclyl (e.g., 4-(N-methyl)aminomethanyl)piperidinyl); B is bicyclic heteroaryl (e.g., 8-fluoro-2-methylimidazo[1,2-a]pyridinyl); L¹ is absent; L² is —C(O)N(R⁴)—(e.g., —C(O)N(H)—); W is N; X is C(R^{3a}) (e.g., CH); Y is C(R^{3a}) (e.g., CH); Z is N; and y is 1. In some embodiments, the compound of Formula (I), (I-a), and (I-e) is Compound 192, or a pharmaceutically acceptable salt, solvate, hydrate, tautomer, or stereoisomer thereof.

As generally described herein, for Formula (II), each of M and P independently refer to C(R²) or N. In some embodiments, each of M and P is independently C(R²) or N. In some embodiments, M and P are each independently C(R²), e.g., CH. In some embodiments, one of M and P is C(R²), and the other of M and P is N. In some embodiments, M is C(R²). In some embodiments, M is N. In some embodiments, P is C(R²). In some embodiments, P is N. In some embodiments, M is C(R²) (e.g., CH) and P is N. In some embodiments, M is N and P is C(R²) (e.g., CH).

In some embodiments,

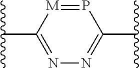

is selected from

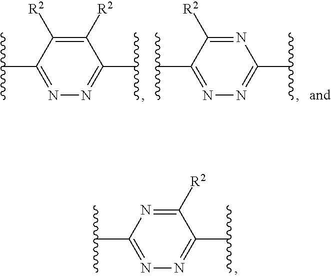

wherein R² is as defined above. In some embodiments, R² is hydrogen.

As generally described herein, for Formula (II), W, X, Y, and Z each independently refer to C(R³) or N. In some embodiments, at least one of W, X, Y, and Z is N. In some embodiments, at least two of W, X, Y, and Z is N. In some embodiments, at least two of X, Y, and Z is N. In some embodiments, at least one of Y and Z is N. In some embodiments, X is N. In some embodiments, X is C(R³) (e.g., CH). In some embodiments, Y is N. In some embodiments, Y is C(R³) (e.g., CH). In some embodiments, Z is N. In some embodiments, Z is C(R³) (e.g., CH).

In some embodiments, W is C(R³). In some embodiments, W is C(R³) (e.g., CH), two of X, Y, and Z are N, and the other of X, Y, and Z is C(R³) (e.g., CH). In some embodiments, W is C(R³) (e.g., CH), one of X, Y, and Z is C(R³) (e.g., CH), and the others of X, Y, and Z are each independently N. In some embodiments, X and Y are each independently N, and W and Z are each independently C(R³) (e.g., CH). In some embodiments, W and X are each independently C(R³) (e.g., CH), and Y and Z are each independently N.

In some embodiments, each of L¹ and L² is independently absent, —N(R³)—(e.g., —N(CH₃)—C₆-C₁₂-arylene, —N(R⁴)C(O)—, or —C(O)N(R⁴)—, wherein arylene is optionally substituted with one or more R¹. In some embodiments, one of L¹ and L² is independently absent, —N(R⁴)C(O)—, or —C(O)N(R⁴)—. In some embodiments, each of L¹ and L² is independently absent. In some embodiments, L¹ is —C(O)N(R⁴)—. In some embodiments, L² is absent. In some embodiments, L¹ is —C(O)NH— and L² is absent.

In some embodiments,

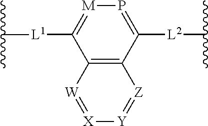

is selected from

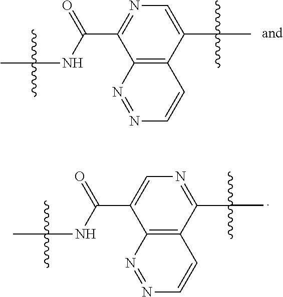 [SMILES: *NC(=O)c1ncc(*)c2ccnnc12 ; *NC(=O)c1cnc(*)c2ccnnc12]

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁶. In some embodiments, R¹ is C₂-C₆-alkenyl substituted with one or more R⁶. In some embodiments, R¹ is C₂-C₆-alkynyl substituted with one or more R⁶. In some embodiments, R¹ is C₁-C₆-heteroalkyl substituted with one or more R⁶. In some embodiments, R¹ is C₁-C₆-haloalkyl substituted with one or more R⁶. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁶. In some embodiments, R¹ is heterocyclyl substituted with one or more R⁶. In some embodiments, R¹ is aryl substituted with one or more R⁶. In some embodiments, R¹ is C₁-C₆ alkylene-aryl substituted with one or more R⁶. In some embodiments, R¹ is C₁-C₆ alkenylene-aryl substituted with one or more R⁶. In some embodiments, R¹ is C₁-C₆ alkylene-heteroaryl substituted with one or more R⁶. In some embodiments, R¹ is heteroaryl substituted with one or more R⁶.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}.

In some embodiments, R¹ is —C(O)R^{D}. In some embodiments, R¹ is —C(O)OR^{D}. In some embodiments, R¹ is-SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁶.

In some embodiments, R² is hydrogen. In some embodiments, R² is C₁-C₆ alkyl. In some embodiments, R² is C₂-C₆-alkenyl. In some embodiments, R² is C₂-C₆-alkynyl. In some embodiments, R² is C₁-alkyl (e.g., methyl). In some embodiments, R² is methyl. In some embodiments, R² is —OR^{A}. In some embodiments, R² is halo (e.g., fluoro, chloro, bromo, or iodo). In some embodiments, R² is fluoro. In some embodiments, R² is cyano.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are each independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁸ (e.g., 1, 2, or 3 R⁸).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, R^{D} is C₁-C₆ alkyl. In some embodiments, R^{D} is hydrogen. In some embodiments, R^{D} is heterocyclyl. In some embodiments, R^{E} is heterocyclyl. In some embodiments, R^{D} is aryl. In some embodiments, R^{E} is aryl. In some embodiments, R^{D} is heteroaryl. In some embodiments, R^{E} is heteroaryl. In some embodiments, R^{D} is C₁-C₆ alkylene-aryl (e.g., benzyl). In some embodiments, R^{E} is C₁-C₆ alkylene-aryl (e.g., benzyl). In some embodiments, R^{D} is C₁-C₆ alkylene-heteroaryl. In some embodiments, R^{E} is C₁-C₆ alkylene-heteroaryl.

In some embodiments, x is 0. In some embodiments, x is 1. In some embodiments, x is 2.

In some embodiments, the compound of Formula (II) is a compound of Formula (II-a):

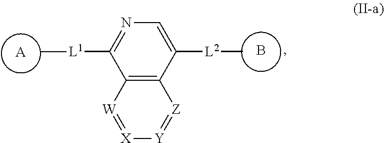

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, Y, and Z are each independently C(R³) or N, wherein at least one of W, X, Y, and Z is N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —N^{B}C(O)R^{D}, —NO₂, —C(O)NR^{B}R^{C}, —C(O)OR^{D}, or —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-b):

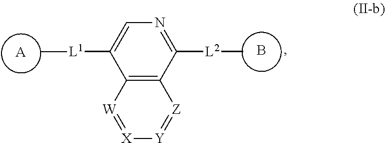

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, Y, and Z are each independently C(R³) or N, wherein at least one of W, X, Y, and Z is N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —N^{B}C(O)R^{D} NO₂, —C(O)NR^{B}R^{C}, —C(O)OR^{D}, or —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-c):

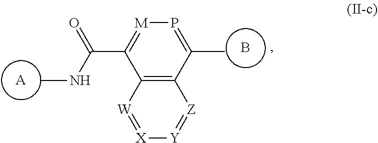

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N, wherein at least one of M and P is independently N; W, X, Y, and Z are each independently C(R³) or N, wherein at least one of W, X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶ each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-d):

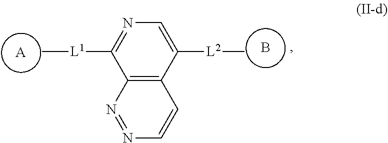

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-e):

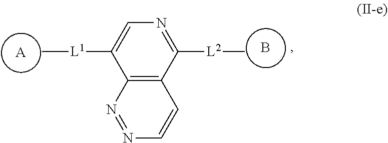

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is selected from a compound in Table 2, or a pharmaceutically acceptable salt thereof.

TABLE 2
Exemplary compounds of Formula (II)

| Com- | |
|---|---|
| pound | |
| No. | Structure |
| 550 | 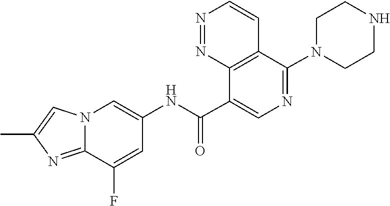 [SMILES: Cc1cn2cc(NC(=O)c3cnc(N4CCNCC4)c4ccnnc34)cc(F)c2n1] |
| 551 | 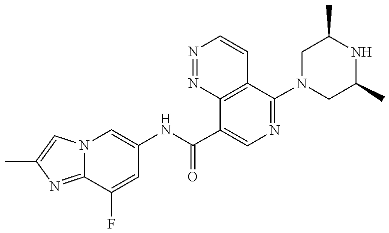 |
| 552 | 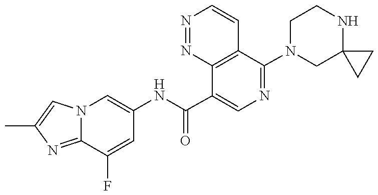 [SMILES: Cc1cn2cc(NC(=O)c3cnc(N4CCNC5(CC5)C4)c4ccnnc34)cc(F)c2n1] |
| 553 | 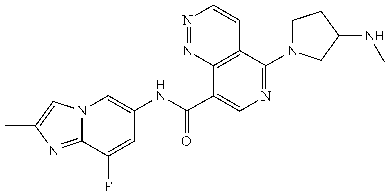 |
| 554 | 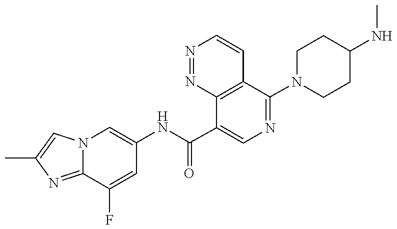 |
| 555 | 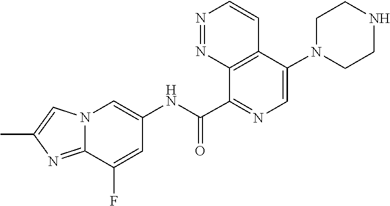 [SMILES: Cc1cn2cc(NC(=O)c3ncc(N4CCNCC4)c4ccnnc34)cc(F)c2n1] |
| 556 | 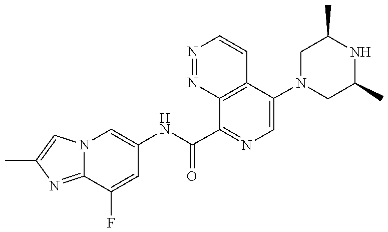 |
| 557 | 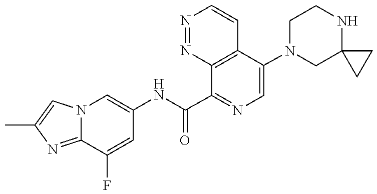 [SMILES: Cc1cn2cc(NC(=O)c3ncc(N4CCNC5(CC5)C4)c4ccnnc34)cc(F)c2n1] |
| 558 | 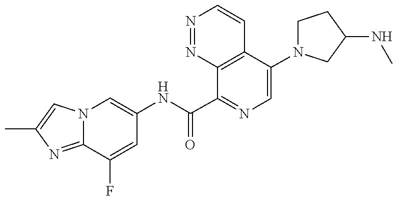 |
| 559 | 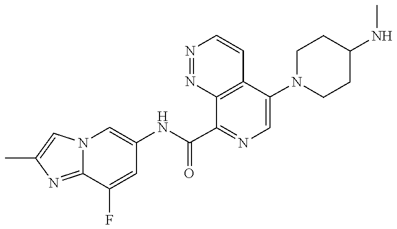 |
| 560 | 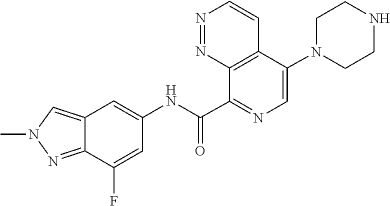 [SMILES: Cn1cc2cc(NC(=O)c3ncc(N4CCNCC4)c4ccnnc34)cc(F)c2n1] |
| 561 | 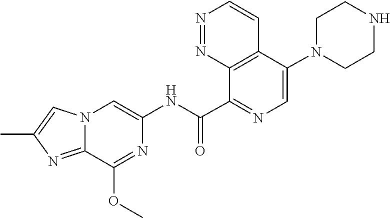 [SMILES: COc1nc(NC(=O)c2ncc(N3CCNCC3)c3ccnnc23)cn2cc(C)nc12] |
| 562 | 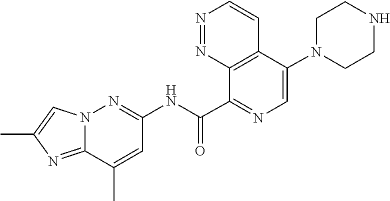 [SMILES: Cc1cn2nc(NC(=O)c3ncc(N4CCNCC4)c4ccnnc34)cc(C)c2n1] |
| 563 | 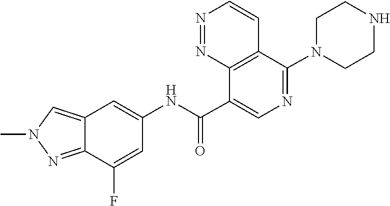 [SMILES: Cn1cc2cc(NC(=O)c3cnc(N4CCNCC4)c4ccnnc34)cc(F)c2n1] |
| 564 | 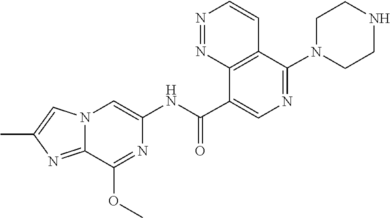 [SMILES: COc1nc(NC(=O)c2cnc(N3CCNCC3)c3ccnnc23)cn2cc(C)nc12] |
| 565 | 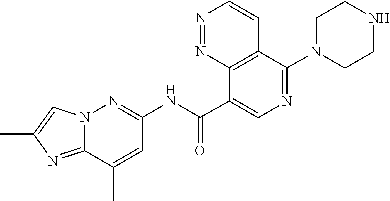 [SMILES: Cc1cn2nc(NC(=O)c3cnc(N4CCNCC4)c4ccnnc34)cc(C)c2n1] |

In another aspect, the present disclosure features a compound of Formula (III):

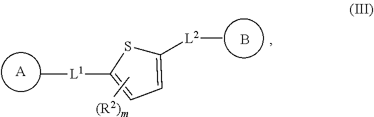

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; m is 0, 1, or 2; and x is 0, 1, or 2.

As generally described herein, for Formula (III), each of L¹ and L² may be independently absent or refer to a C₁-C₆-alkylene, C₁-C₆-heteroalkylene, —O—, —C(O)—, —N(R³)—, —N(R³)C(O)—, or —C(O)N(R³)— group, wherein each alkylene and heteroalkylene is optionally substituted with one or more R⁴.

In some embodiments, L¹ is absent. In some embodiments, L¹ is C₁-C₆-alkylene (e.g., C₁-alkylene, C₂-alkylene, C₃-alkylene, C₄-alkylene, C₅-alkylene, or C₆-alkylene). In some embodiments, L¹ is unsubstituted C₁-C₆ alkylene. In some embodiments, L¹ is substituted C₁-C₆-alkylene, e.g., C₁-C₆ alkylene substituted with one or more R⁴. In some embodiments, L¹ is C₁-alkylene substituted with one R⁴. In some embodiments, L¹ is —CH₂— (or methylene). In some embodiments, L¹ is —C(O)— (or carbonyl).

In some embodiments, L¹ is absent, C₁-C₆-alkylene, C₁-C₆-heteroalkylene, —N(R³)C(O)—, or —C(O)N(R³)—, wherein each alkylene and heteroalkylene is optionally substituted with one or more R⁴.

In some embodiments, L² is absent. In some embodiments, L² is C₁-C₆-alkylene (e.g., C₁-alkylene, C₂-alkylene, C₃-alkylene, C₄-alkylene, C₅-alkylene, or C₆-alkylene). In some embodiments, L² is unsubstituted C₁-C₆ alkylene. In some embodiments, L² is substituted C₁-C₆-alkylene, e.g., C₁-C₆ alkylene substituted with one or more R⁴. In some embodiments, L² is C₁-alkylene substituted with one R⁴. In some embodiments, L² is —CH₂— (or methylene). In some embodiments, L² is —C(O)— (or carbonyl).

In some embodiments, L² is absent, C₁-C₆-alkylene, C₁-C₆-heteroalkylene, —N(R³)C(O)—, or —C(O)N(R³)—, wherein each alkylene and heteroalkylene is optionally substituted with one or more R⁴.

In some embodiments, L¹ is C₁-C₆-heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L¹ is unsubstituted C₁-C₆ heteroalkylene. In some embodiments, L¹ is C₁-C₆-heteroalkylene substituted with one or more R⁴. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L¹ is absent, —N(R³)C(O)—, or —C(O)N(R³)—. In some embodiments, L¹ is —N(R³)C(O)—. In some embodiments, L¹ is —C(O)N(R³)—.

In some embodiments, L² is C₁-C₆-heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L² is unsubstituted C₁-C₆ heteroalkylene. In some embodiments, L² is C₁-C₆-heteroalkylene substituted with one or more R⁴. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L² is absent, —N(R³)C(O)—, or —C(O)N(R³)—. In some embodiments, L² is —N(R³)C(O)—. In some embodiments, L² is —C(O)N(R³)—.

In some embodiments, each of L¹ and L² are not both absent. In some embodiments, L¹ is absent and L² is —N(R³)C(O)— or —C(O)N(R³)—. In some embodiments, L² is absent and L¹ is —N(R³)C(O)— or —C(O)N(R³)—.

In some embodiments, L¹ is oxygen. In some embodiments, L¹ is nitrogen, which may be substituted with R³. In some embodiments, L¹ is nitrogen substituted with one R³. In some embodiments, L¹ is —N(R³)—. In some embodiments, L¹ is —N(CH₃)—. In some embodiments, L¹ is —NH—. In some embodiments, L¹ is —O—.

In some embodiments, L² is oxygen. In some embodiments, L² is nitrogen, which may be substituted with R³. In some embodiments, L² is nitrogen substituted with one R³. In some embodiments, L² is —N(R³)—. In some embodiments, L² is —N(CH₃)—. In some embodiments, L² is —NH—. In some embodiments, L² is —O—.

In some embodiments

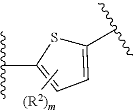

is selected from

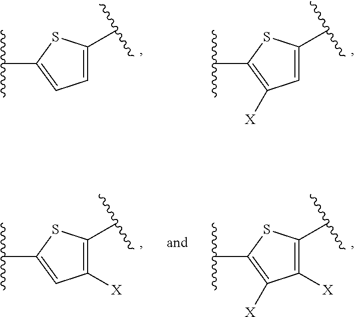

wherein each X is independently halo (e.g., fluoro, chloro bromo, or iodo). In some embodiments,

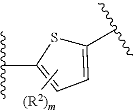

is

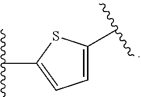 [SMILES: *c1ccc(*)s1]

In some embodiments,

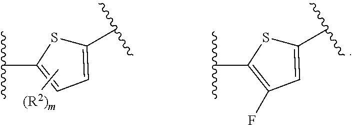

In some embodiments,

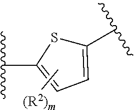

is

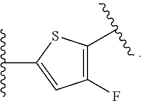 [SMILES: *c1cc(F)c(*)s1]

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁵. In some embodiments, R¹ is C₂-C₆-alkenyl substituted with one or more R⁵. In some embodiments, R¹ is C₂-C₆-alkynyl substituted with one or more R⁵. In some embodiments, R¹ is C₁-C₆-heteroalkyl substituted with one or more R⁵. In some embodiments, R¹ is C₁-C₆-haloalkyl substituted with one or more R⁵. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁵. In some embodiments, R¹ is heterocyclyl substituted with one or more R⁵. In some embodiments, R¹ is aryl substituted with one or more R⁵. In some embodiments, R¹ is C₁-C₆ alkylene-aryl substituted with one or more R⁵. In some embodiments, R¹ is C₁-C₆ alkenylene-aryl substituted with one or more R⁵. In some embodiments, R¹ is C₁-C₆ alkylene-heteroaryl substituted with one or more R⁵. In some embodiments, R¹ is heteroaryl substituted with one or more R⁵.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}. In some embodiments, R¹ is —C(O)R^{D}. In some embodiments, R¹ is —C(O)OR^{D}. In some embodiments, R¹ is-SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁵.

In some embodiments, R² is C₁-C₆-alkyl. In some embodiments, R² is C₂-C₆-alkenyl. In some embodiments, R² is C₂-C₆-alkynyl. In some embodiments, R² is halo (e.g., fluoro, chloro, bromo, or iodo). In some embodiments, R² is fluoro. In some embodiments, R² is cyano. In some embodiments, R² is —OR^{A} (e.g., —OH).

In some embodiments, R³ is hydrogen. In some embodiments, R³ is C₁-C₆ alkyl. In some embodiments, R³ is C₁-C₆ haloalkyl. In some embodiments, R³ is methyl.

In some embodiments, R⁴ is C₁-C₆-alkyl. In some embodiments, R⁴ is C₁-C₆-heteroalkyl. In some embodiments, R⁴ is C₁-C₆-haloalkyl (e.g., —CF₃ or —CHF₂). In some embodiments, R⁴ is cycloalkyl. In some embodiments, R⁴ is halo (e.g., fluoro, chloro, bromo, or iodo). In some embodiments, R⁴ is cyano. In some embodiments, R⁴ is oxo. In some embodiments, R⁴ is —OR^{A}. In some embodiments, R⁴ is —NR^{B}R^{C}. In some embodiments, R⁴ is —C(O)R^{D} or —C(O)OR^{D}.

In some embodiments, R⁵ is C₁-C₆-alkyl. In some embodiments, R⁵ is C₂-C₆-alkenyl. In some embodiments, R⁵ is C₂-C₆-alkynyl. In some embodiments, R⁵ is C₁-C₆-heteroalkyl. In some embodiments, R⁵ is C₁-C₆-haloalkyl. In some embodiments, R⁵ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁵ is C₁-C₆-alkyl substituted with one or more R¹¹. In some embodiments, R⁵ is C₂-C₆-alkenyl substituted with one or more R¹¹. In some embodiments, R⁵ is C₂-C₆-alkynyl substituted with one or more R¹¹. In some embodiments, R⁵ is C₁-C₆-haloalkyl substituted with one or more R¹¹. In some embodiments, R⁵ is C₁-C₆-heteroalkyl substituted with one or more R¹¹.

In some embodiments, R⁵ is cycloalkyl. In some embodiments, R⁵ is heterocyclyl. In some embodiments, R⁵ is aryl. In some embodiments, R⁵ is heteroaryl. In some embodiments, R⁵ is unsubstituted cycloalkyl, unsubstituted heterocyclyl, unsubstituted aryl, or unsubstituted heteroaryl. In some embodiments, R⁵ is cycloalkyl substituted with one or more R¹¹. In some embodiments, R⁵ is heterocyclyl substituted with one or more R¹¹. In some embodiments, R⁵ is aryl substituted with one or more R¹¹. In some embodiments, R⁵ is heteroaryl substituted with one or more R¹¹.

In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}. In some embodiments, R⁵ is —NR^{B}R^{C}. In some embodiments, R⁵ is —NR^{B}C(O)R^{D}. In some embodiments, R⁵ is —NO₂. In some embodiments, R⁵ is —C(O)NR^{B}R^{C}. In some embodiments, R⁵ is —C(O)R^{D}. In some embodiments, R⁵ is —C(O)OR^{D}. In some embodiments, R⁵ is —SR^{E}. In some embodiments, R⁵ is —S(O)ₓR^{D}.

In some embodiments, R⁷ is C₁-C₆-alkyl. In some embodiments, R⁷ is halo (e.g., fluoro, chloro, bromo, or iodo).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃ or —CHF₂). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)R^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁷ (e.g., 1, 2, or 3 R⁷).

In some embodiments, R^{D}, R^{E}, or both are independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, each of R^{D} and R^{E} is independently C₁-C₆ alkyl. In some embodiments, R^{D} is hydrogen. In some embodiments, R^{E} is hydrogen. In some embodiments, R^{D} is C₁-C₆ alkyl (e.g., methyl). In some embodiments, R^{E} is C₁-C₆ alkyl (e.g., methyl). In some embodiments, R^{D} is C₁-C₆ heteroalkyl. In some embodiments, R^{E} is C₁-C₆ heteroalkyl. In some embodiments, R^{D} is C₁-C₆ haloalkyl. In some embodiments, R^{E} is C₁-C₆ haloalkyl. In some embodiments, R^{D} is cycloalkyl. In some embodiments, R^{E} is cycloalkyl. In some embodiments, R^{D} is heterocyclyl. In some embodiments, R^{E} is heterocyclyl. In some embodiments, R^{D} is aryl. In some embodiments, R^{E} is aryl. In some embodiments, R^{D} is heteroaryl. In some embodiments, R^{E} is heteroaryl. In some embodiments, R^{D} is C₁-C₆ alkylene-aryl (e.g., benzyl). In some embodiments, R^{E} is C₁-C₆ alkylene-aryl (e.g., benzyl). In some embodiments, R^{D} is C₁-C₆ alkylene-heteroaryl. In some embodiments, R^{E} is C₁-C₆ alkylene-heteroaryl.

In some embodiments, m is an integer between 0 and 2 (e.g., 0, 1, or 2). In some embodiments, m is 0 or 1. In some embodiments, m is 0. In some embodiments, m is 1. In some embodiments, m is 1 and R² is halo. In some embodiments, m is 1 and R² is fluoro. In some embodiments, m is 2. In some embodiments, x is an integer between 0 and 2 (e.g., 0, 1, or 2). In some embodiments, x is 0. In some embodiments, x is 1. In some embodiments, x is 2.

In some embodiments, the compound of Formula (III) is a compound of Formula (III-a):

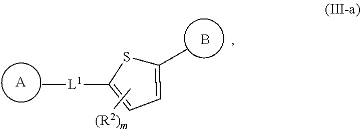

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; m is 0, 1, or 2; and x is 0, 1, or 2.

In some embodiments, the compound of Formula (III) is a compound of Formula (III-b):

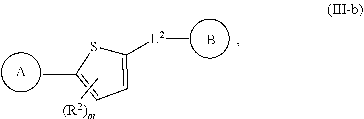

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; m is 0, 1, or 2; and x is 0, 1, or 2.

In some embodiments, the compound of Formula (III) is a compound of Formula (III-c)

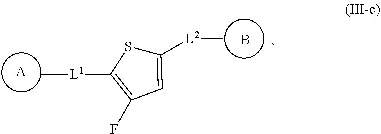

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; and x is 0, 1, or 2.

In some embodiments the compound of Formula (III) is a compound of Formula (III-d):

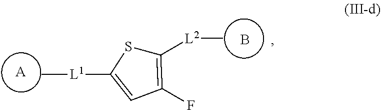

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; o two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; and x is 0, 1, or 2.

In some embodiments, the compound of Formula (III) is a compound of Formula (III-e):

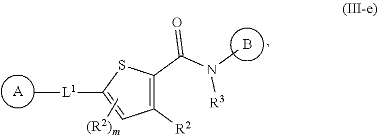

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; m is 0, 1, or 2; and x is 0, 1, or 2.

In some embodiments, the compound of Formula (III) is a compound of Formula (III-f):

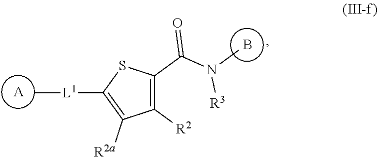

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D} NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{2a} is hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; and x is 0, 1, or 2.

In some embodiments, the compound of Formula (III) is a compound of Formula (III-g):

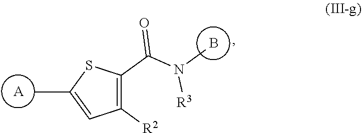

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; and x is 0, 1, or 2.

In some embodiments, the compound of Formula (III) is a compound of Formula (III-h):

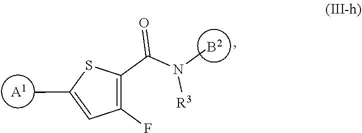

or a pharmaceutically acceptable salt, solvate, hydrate, tautomer, or stereoisomer thereof, wherein A¹ is monocyclic or bicyclic heterocyclyl, each of which is optionally substituted with one or more R¹; B¹ is monocyclic or bicyclic heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R³ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; and x is 0, 1, or 2.

In some embodiments for Formula (III), A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted piperazinyl. In some embodiments, A is

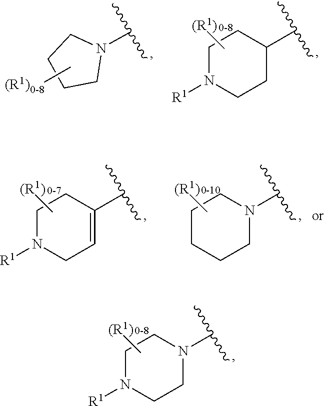

wherein each R¹ is independently hydrogen or C₁-C₆-alkyl. In some embodiments, A is

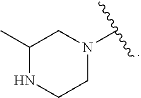 [SMILES: *N1CCNC(C)C1]

In some embodiments, A is

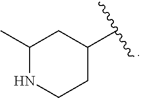 [SMILES: *C1CCNC(C)C1]

In some embodiments, A is

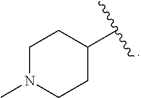 [SMILES: *C1CCN(C)CC1]

In some embodiments, A is

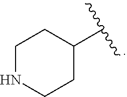 [SMILES: *C1CCNCC1]

In some embodiments, A is

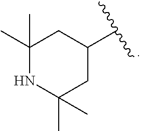 [SMILES: *C1CC(C)(C)NC(C)(C)C1]

In some embodiments, B is heteroaryl optionally substituted with one or more R¹. In some embodiments, B is monocyclic heteroaryl. In some embodiments, B is bicyclic heteroaryl. In some embodiments, B is monocyclic nitrogen-containing heteroaryl. In some embodiments, B is bicyclic nitrogen-containing heteroaryl.

In some embodiments, B is selected from

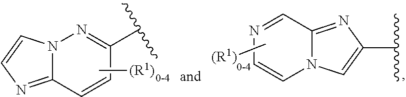

and wherein each R¹ is as defined above. In some embodiments, B is

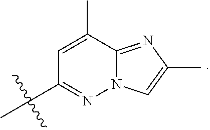 [SMILES: *c1cc(C)c2nc(C)cn2n1]

In some embodiments, B is

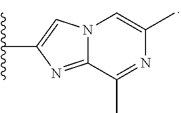 [SMILES: *c1cn2cc(C)nc(C)c2n1]

In some embodiments, the compound of Formula (III) is a compound of Formula (III-i):

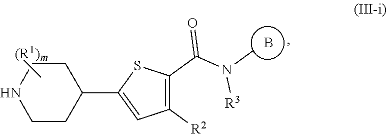

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; m is 0, 1, 2, 3, or 4; and x is 0, 1, or 2.

In some embodiments, the compound of Formula (III) is a compound of Formula (III-j):

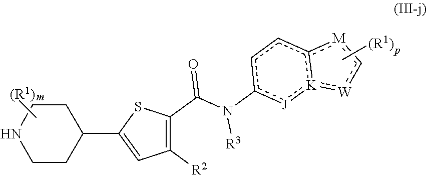

or a pharmaceutically acceptable salt, solvate, hydrate, tautomer, or stereoisomer thereof, wherein: each of J, K, and M is selected from N and C(R′); W is selected from 0 and C(R′); at least one of J, K, and M is N and the bonds in the ring comprising J, K, and M may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R′ is hydrogen, halo, or C₁-C₆-alkyl; p is 0, 1, 2, 3, or 4; and x is 0, 1, or 2.

In some embodiments, the compound of Formula (III) is a compound of Formula (III-k):

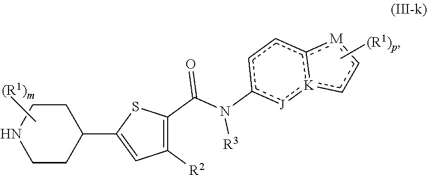

or a pharmaceutically acceptable salt, solvate, hydrate, tautomer, or stereoisomer thereof, wherein each of J, K, and M is selected from N and C(R′); at least one of J, K, and M is N and the bonds in the ring comprising J, K, and M may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵; R² is C₁-C₆-alkyl, C₂-C₆-alkenyl, C₂-C₆-alkynyl, C₁-C₆-heteroalkyl, C₁-C₆-haloalkyl, halo, cyano, or —OR^{A}; R³ is hydrogen, C₁-C₆-alkyl, or C₁-C₆-haloalkyl; each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is independently C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; R′ is hydrogen, halo, or C₁-C₆-alkyl; p is 0, 1, 2, 3, or 4; and x is 0, 1, or 2.

In some embodiments, the compound of Formula (III) is selected from a compound in Table 3, or a pharmaceutically acceptable salt, solvate, hydrate, tautomer, or stereoisomer thereof.

TABLE 3
Exemplary compounds of Formula (III).

| Compound No. | Structure |
|---|---|
| 575 | 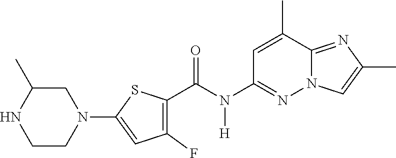 [SMILES: Cc1cn2nc(NC(=O)c3sc(N4CCNC(C)C4)cc3F)cc(C)c2n1] |
| 576 | 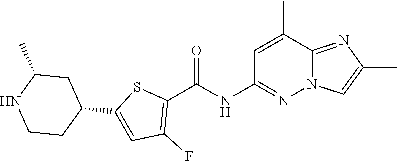 [SMILES: Cc1cn2nc(NC(=O)c3sc([C@@H]4CCN[C@H](C)C4)cc3F)cc(C)c2n1] |
| 577 | 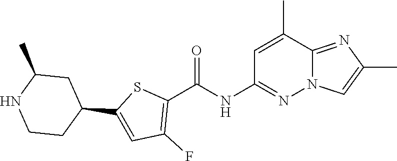 [SMILES: Cc1cn2nc(NC(=O)c3sc([C@H]4CCN[C@@H](C)C4)cc3F)cc(C)c2n1] |
| 578 | 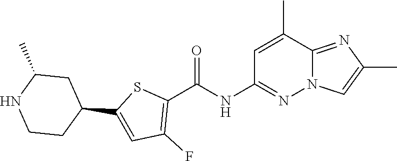 [SMILES: Cc1cn2nc(NC(=O)c3sc([C@H]4CCN[C@H](C)C4)cc3F)cc(C)c2n1] |
| 579 | 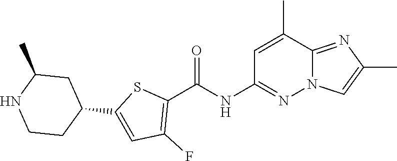 [SMILES: Cc1cn2nc(NC(=O)c3sc([C@@H]4CCN[C@@H](C)C4)cc3F)cc(C)c2n1] |
| 580 | 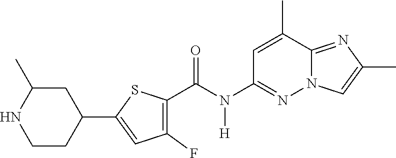 [SMILES: Cc1cn2nc(NC(=O)c3sc(C4CCNC(C)C4)cc3F)cc(C)c2n1] |
| 581 | 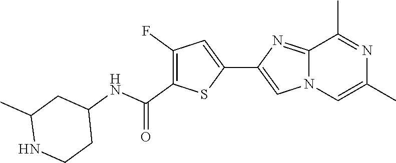 |
| 582 | 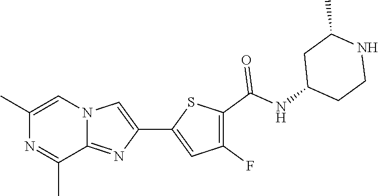 [SMILES: Cc1cn2cc(-c3cc(F)c(C(=O)N[C@H]4CCN[C@@H](C)C4)s3)nc2c(C)n1] |
| 583 | 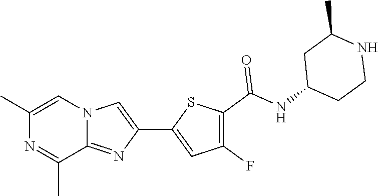 [SMILES: Cc1cn2cc(-c3cc(F)c(C(=O)N[C@H]4CCN[C@H](C)C4)s3)nc2c(C)n1] |
| 584 | 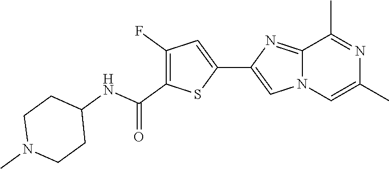 |
| 585 | 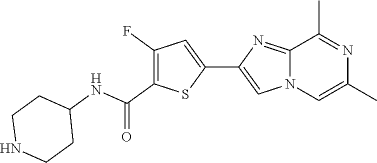 [SMILES: Cc1cn2cc(-c3cc(F)c(C(=O)NC4CCNCC4)s3)nc2c(C)n1] |
| 586 | 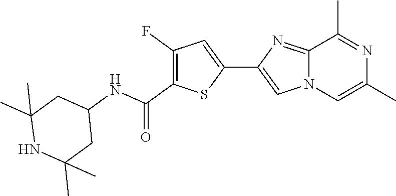 |
| 587 | 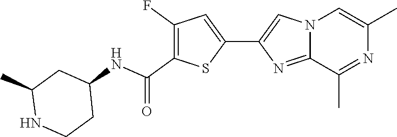 |
| 588 | 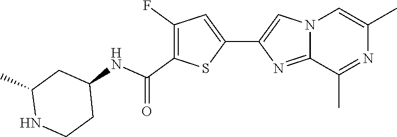 |
| 589 | 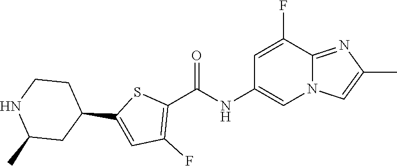 |
| 590 | 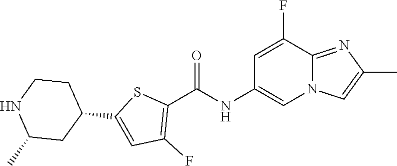 |
| 591 | 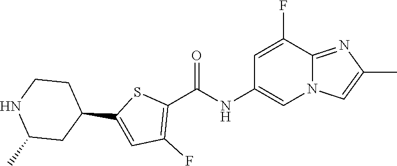 |
| 592 | 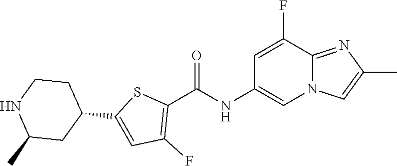 |
| 593 | 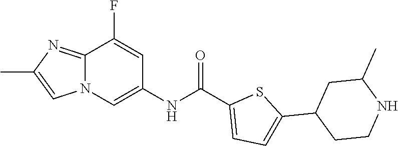 [SMILES: Cc1cn2cc(NC(=O)c3ccc(C4CCNC(C)C4)s3)cc(F)c2n1] |
| 594 | 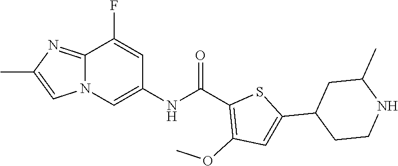 [SMILES: COc1cc(C2CCNC(C)C2)sc1C(=O)Nc1cc(F)c2nc(C)cn2c1] |
| 595 | 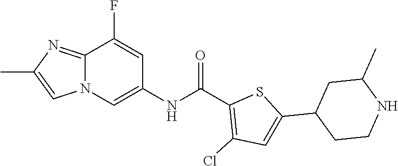 [SMILES: Cc1cn2cc(NC(=O)c3sc(C4CCNC(C)C4)cc3Cl)cc(F)c2n1] |
| 596 | 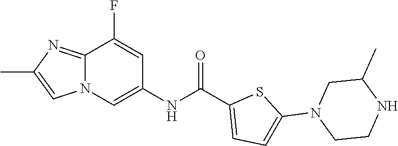 [SMILES: Cc1cn2cc(NC(=O)c3ccc(N4CCNC(C)C4)s3)cc(F)c2n1] |
| 597 | 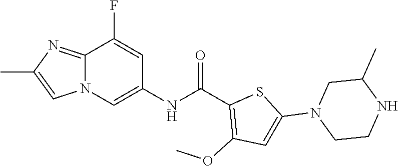 [SMILES: COc1cc(N2CCNC(C)C2)sc1C(=O)Nc1cc(F)c2nc(C)cn2c1] |
| 598 | 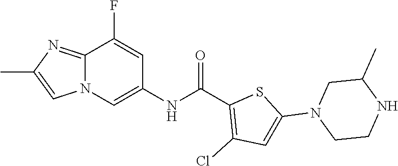 [SMILES: Cc1cn2cc(NC(=O)c3sc(N4CCNC(C)C4)cc3Cl)cc(F)c2n1] |
| 599 | 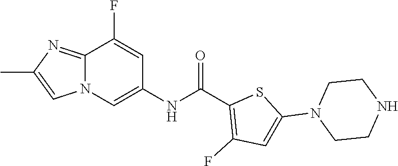 [SMILES: Cc1cn2cc(NC(=O)c3sc(N4CCNCC4)cc3F)cc(F)c2n1] |
| 600 | 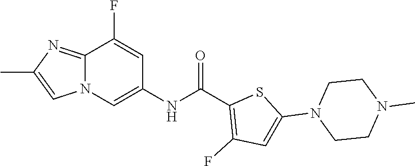 |
| 601 | 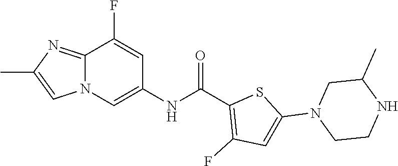 [SMILES: Cc1cn2cc(NC(=O)c3sc(N4CCNC(C)C4)cc3F)cc(F)c2n1] |
| 602 | 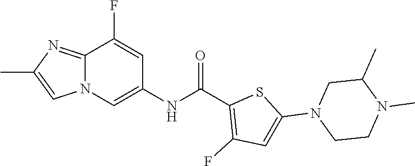 |
| 603 | 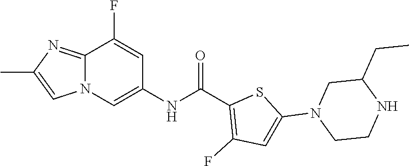 [SMILES: CCC1CN(c2cc(F)c(C(=O)Nc3cc(F)c4nc(C)cn4c3)s2)CCN1] |
| 604 | 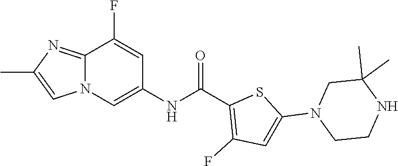 |
| 605 | 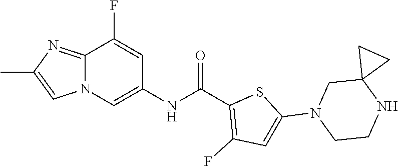 [SMILES: Cc1cn2cc(NC(=O)c3sc(N4CCNC5(CC5)C4)cc3F)cc(F)c2n1] |
| 606 | 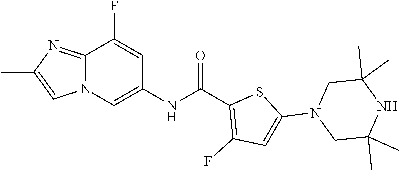 |
| 607 | 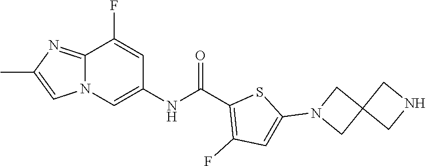 [SMILES: Cc1cn2cc(NC(=O)c3sc(N4CC5(CNC5)C4)cc3F)cc(F)c2n1] |
| 608 | 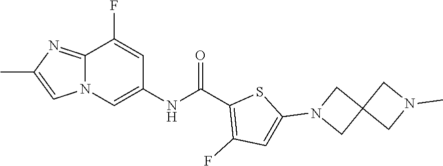 |
| 609 | 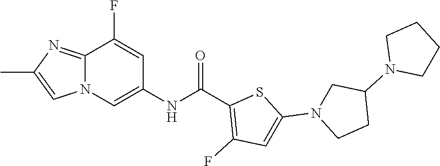 [SMILES: Cc1cn2cc(NC(=O)c3sc(N4CCC(N5CCCC5)C4)cc3F)cc(F)c2n1] |
| 610 | 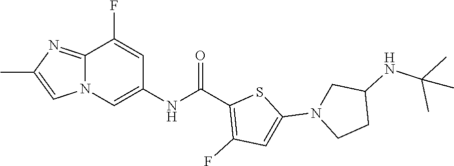 [SMILES: Cc1cn2cc(NC(=O)c3sc(N4CCC(NC(C)(C)C)C4)cc3F)cc(F)c2n1] |
| 611 | 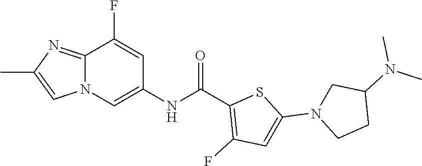 |
| 612 | 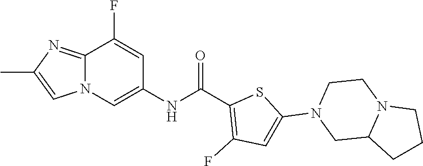 [SMILES: Cc1cn2cc(NC(=O)c3sc(N4CCN5CCCC5C4)cc3F)cc(F)c2n1] |
| 613 | 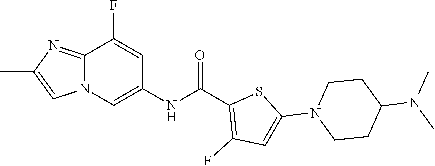 |
| 614 | 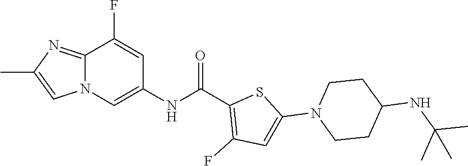 [SMILES: Cc1cn2cc(NC(=O)c3sc(N4CCC(NC(C)(C)C)CC4)cc3F)cc(F)c2n1] |
| 615 | 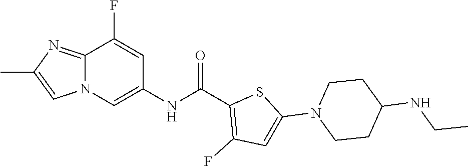 [SMILES: CCNC1CCN(c2cc(F)c(C(=O)Nc3cc(F)c4nc(C)cn4c3)s2)CC1] |
| 616 | 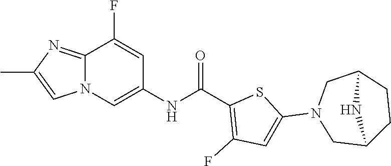 [SMILES: Cc1cn2cc(NC(=O)c3sc(N4C[C@H]5CC[C@@H](C4)N5)cc3F)cc(F)c2n1] |
| 617 | 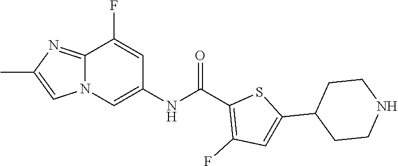 [SMILES: Cc1cn2cc(NC(=O)c3sc(C4CCNCC4)cc3F)cc(F)c2n1] |
| 618 | 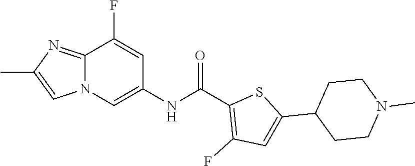 |
| 619 | 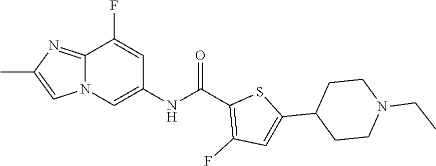 [SMILES: CCN1CCC(c2cc(F)c(C(=O)Nc3cc(F)c4nc(C)cn4c3)s2)CC1] |
| 620 | 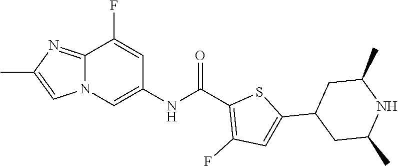 |
| 621 | 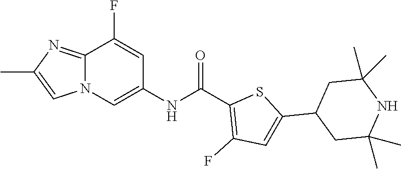 |
| 622 | 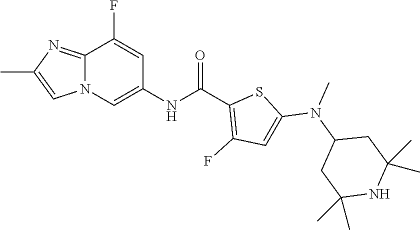 |
| 623 | 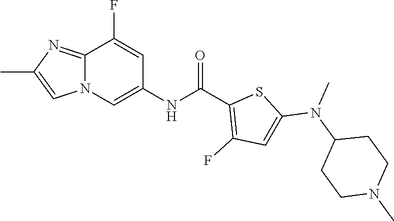 |
| 624 | 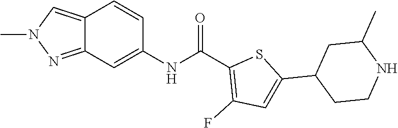 [SMILES: CC1CC(c2cc(F)c(C(=O)Nc3ccc4cn(C)nc4c3)s2)CCN1] |
| 625 | 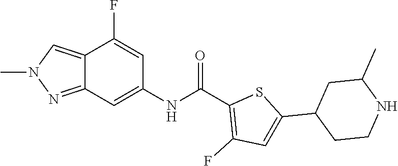 [SMILES: CC1CC(c2cc(F)c(C(=O)Nc3cc(F)c4cn(C)nc4c3)s2)CCN1] |
| 626 | 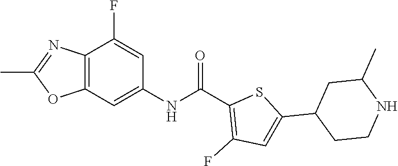 [SMILES: Cc1nc2c(F)cc(NC(=O)c3sc(C4CCNC(C)C4)cc3F)cc2o1] |
| 627 | 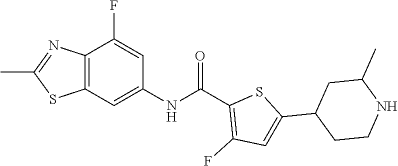 [SMILES: Cc1nc2c(F)cc(NC(=O)c3sc(C4CCNC(C)C4)cc3F)cc2s1] |
| 628 | 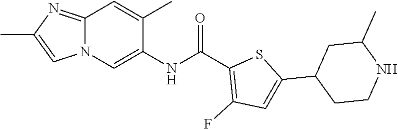 [SMILES: Cc1cn2cc(NC(=O)c3sc(C4CCNC(C)C4)cc3F)c(C)cc2n1] |
| 629 | 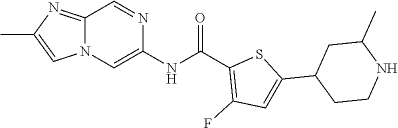 [SMILES: Cc1cn2cc(NC(=O)c3sc(C4CCNC(C)C4)cc3F)ncc2n1] |
| 630 | 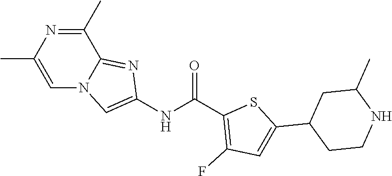 [SMILES: Cc1cn2cc(NC(=O)c3sc(C4CCNC(C)C4)cc3F)nc2c(C)n1] |
| 631 | 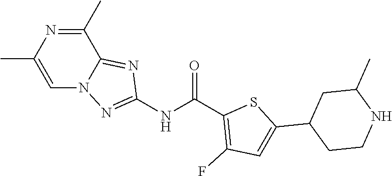 [SMILES: Cc1cn2nc(NC(=O)c3sc(C4CCNC(C)C4)cc3F)nc2c(C)n1] |
| 632 | 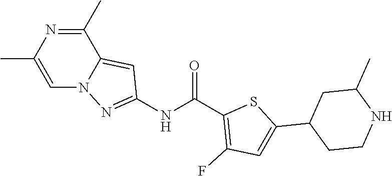 [SMILES: Cc1cn2nc(NC(=O)c3sc(C4CCNC(C)C4)cc3F)cc2c(C)n1] |
| 633 | 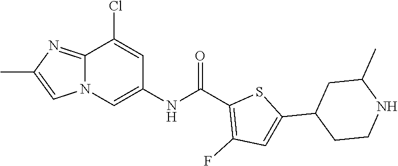 [SMILES: Cc1cn2cc(NC(=O)c3sc(C4CCNC(C)C4)cc3F)cc(Cl)c2n1] |
| 634 | 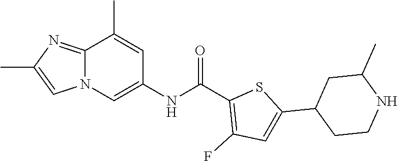 [SMILES: Cc1cn2cc(NC(=O)c3sc(C4CCNC(C)C4)cc3F)cc(C)c2n1] |
| 635 | 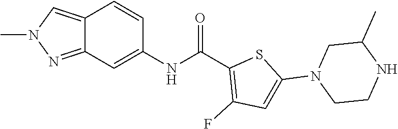 [SMILES: CC1CN(c2cc(F)c(C(=O)Nc3ccc4cn(C)nc4c3)s2)CCN1] |
| 636 | 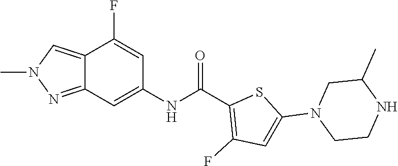 [SMILES: CC1CN(c2cc(F)c(C(=O)Nc3cc(F)c4cn(C)nc4c3)s2)CCN1] |
| 637 | 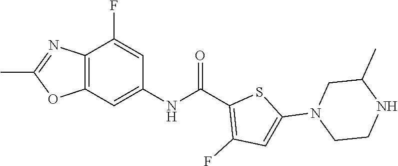 [SMILES: Cc1nc2c(F)cc(NC(=O)c3sc(N4CCNC(C)C4)cc3F)cc2o1] |
| 638 | 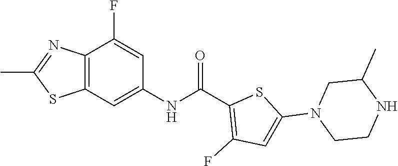 [SMILES: Cc1nc2c(F)cc(NC(=O)c3sc(N4CCNC(C)C4)cc3F)cc2s1] |
| 639 | 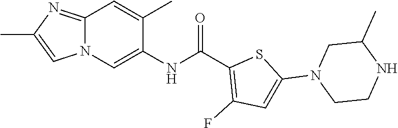 [SMILES: Cc1cn2cc(NC(=O)c3sc(N4CCNC(C)C4)cc3F)c(C)cc2n1] |
| 640 | 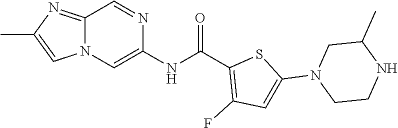 [SMILES: Cc1cn2cc(NC(=O)c3sc(N4CCNC(C)C4)cc3F)ncc2n1] |
| 641 | 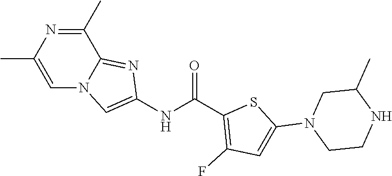 [SMILES: Cc1cn2cc(NC(=O)c3sc(N4CCNC(C)C4)cc3F)nc2c(C)n1] |
| 642 | 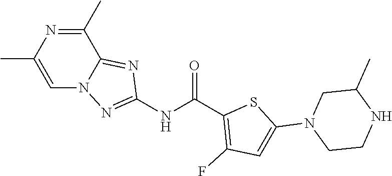 [SMILES: Cc1cn2nc(NC(=O)c3sc(N4CCNC(C)C4)cc3F)nc2c(C)n1] |
| 643 | 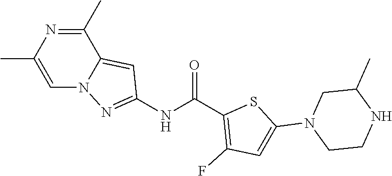 [SMILES: Cc1cn2nc(NC(=O)c3sc(N4CCNC(C)C4)cc3F)cc2c(C)n1] |
| 644 | 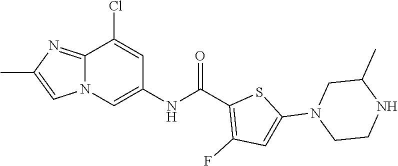 [SMILES: Cc1cn2cc(NC(=O)c3sc(N4CCNC(C)C4)cc3F)cc(Cl)c2n1] |
| 645 | 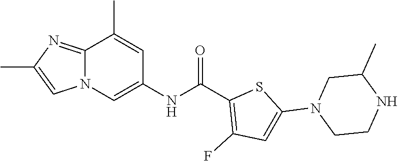 [SMILES: Cc1cn2cc(NC(=O)c3sc(N4CCNC(C)C4)cc3F)cc(C)c2n1] |
| 646 | 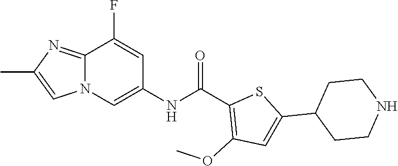 [SMILES: COc1cc(C2CCNCC2)sc1C(=O)Nc1cc(F)c2nc(C)cn2c1] |
| 647 | 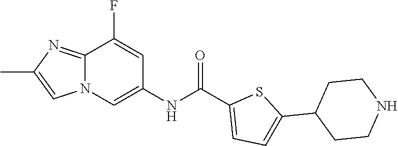 [SMILES: Cc1cn2cc(NC(=O)c3ccc(C4CCNCC4)s3)cc(F)c2n1] |
| 648 | 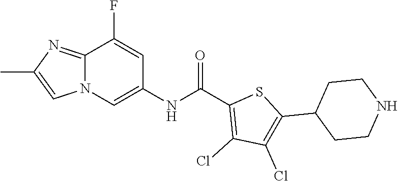 [SMILES: Cc1cn2cc(NC(=O)c3sc(C4CCNCC4)c(Cl)c3Cl)cc(F)c2n1] |
| 649 | 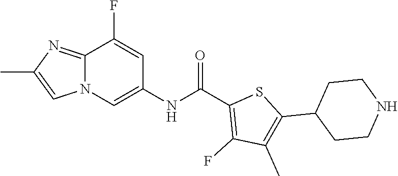 [SMILES: Cc1cn2cc(NC(=O)c3sc(C4CCNCC4)c(C)c3F)cc(F)c2n1] |
| 650 | 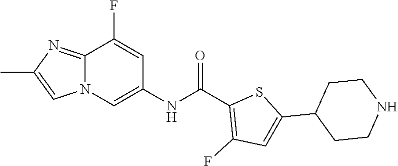 [SMILES: Cc1cn2cc(NC(=O)c3sc(C4CCNCC4)cc3F)cc(F)c2n1] |
| 651 | 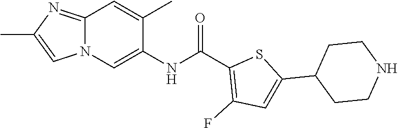 [SMILES: Cc1cn2cc(NC(=O)c3sc(C4CCNCC4)cc3F)c(C)cc2n1] |
| 652 | 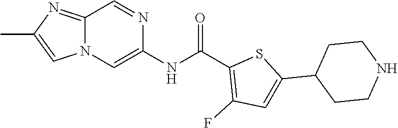 [SMILES: Cc1cn2cc(NC(=O)c3sc(C4CCNCC4)cc3F)ncc2n1] |
| 653 | 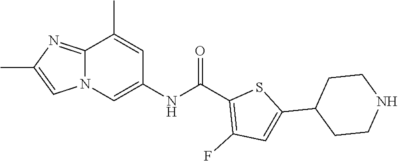 [SMILES: Cc1cn2cc(NC(=O)c3sc(C4CCNCC4)cc3F)cc(C)c2n1] |
| 654 | 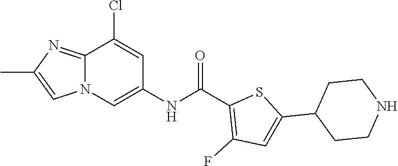 [SMILES: Cc1cn2cc(NC(=O)c3sc(C4CCNCC4)cc3F)cc(Cl)c2n1] |
| 655 | 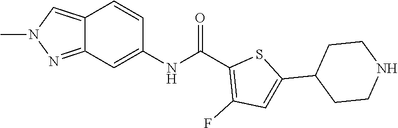 [SMILES: Cn1cc2ccc(NC(=O)c3sc(C4CCNCC4)cc3F)cc2n1] |
| 656 | 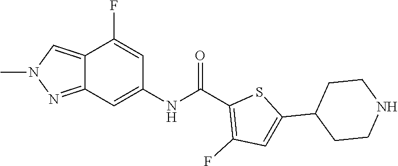 [SMILES: Cn1cc2c(F)cc(NC(=O)c3sc(C4CCNCC4)cc3F)cc2n1] |
| 657 | 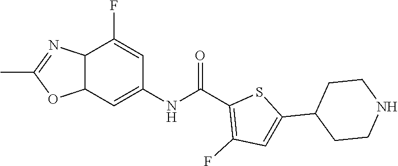 [SMILES: CC1=NC2C(F)=CC(NC(=O)c3sc(C4CCNCC4)cc3F)=CC2O1] |
| 658 | 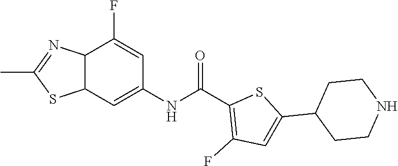 [SMILES: CC1=NC2C(F)=CC(NC(=O)c3sc(C4CCNCC4)cc3F)=CC2S1] |
| 659 | 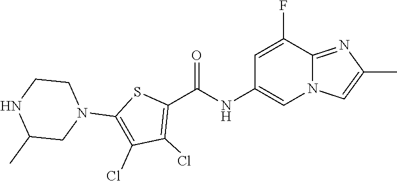 |
| 660 | 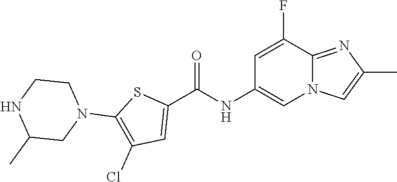 |
| 661 | 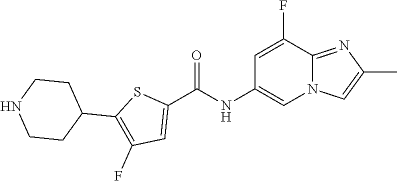 [SMILES: Cc1cn2cc(NC(=O)c3cc(F)c(C4CCNCC4)s3)cc(F)c2n1] |
| 662 | 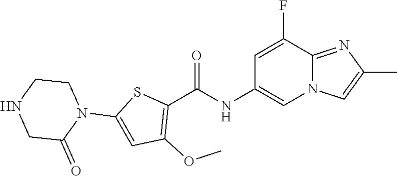 [SMILES: COc1cc(N2CCNCC2=O)sc1C(=O)Nc1cc(F)c2nc(C)cn2c1] |
| 663 | 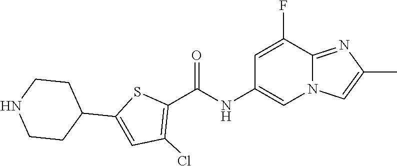 [SMILES: Cc1cn2cc(NC(=O)c3sc(C4CCNCC4)cc3Cl)cc(F)c2n1] |
| 664 | 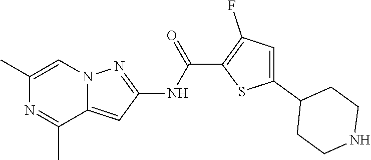 [SMILES: Cc1cn2nc(NC(=O)c3sc(C4CCNCC4)cc3F)cc2c(C)n1] |
| 665 | 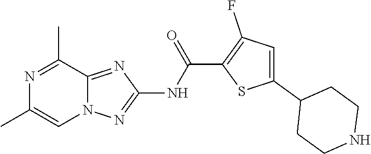 [SMILES: Cc1cn2nc(NC(=O)c3sc(C4CCNCC4)cc3F)nc2c(C)n1] |
| 666 | 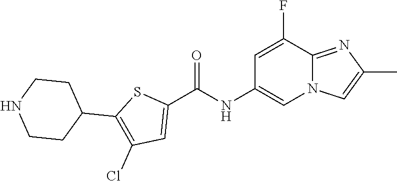 [SMILES: Cc1cn2cc(NC(=O)c3cc(Cl)c(C4CCNCC4)s3)cc(F)c2n1] |
| 667 | 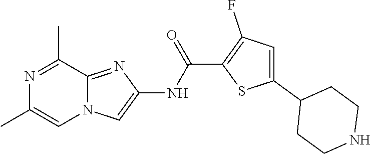 [SMILES: Cc1cn2cc(NC(=O)c3sc(C4CCNCC4)cc3F)nc2c(C)n1] |
| 668 | 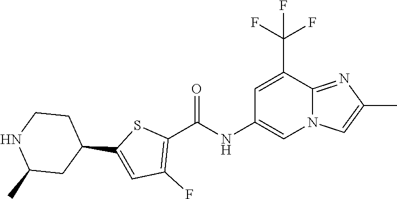 |
| 669 | 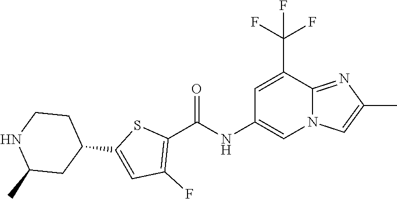 |
| 670 | 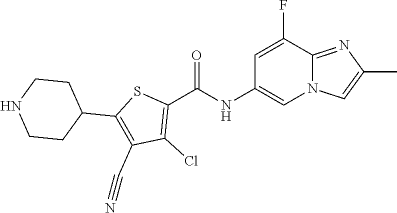 [SMILES: Cc1cn2cc(NC(=O)c3sc(C4CCNCC4)c(C#N)c3Cl)cc(F)c2n1] |
| 671 | 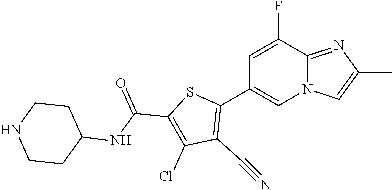 [SMILES: Cc1cn2cc(-c3sc(C(=O)NC4CCNCC4)c(Cl)c3C#N)cc(F)c2n1] |
| 672 | 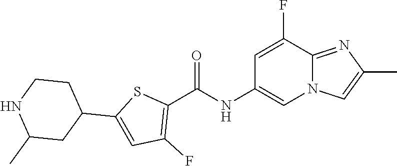 |
| 673 | 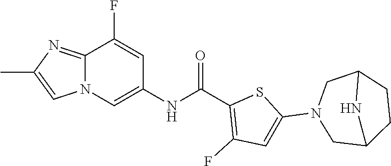 [SMILES: Cc1cn2cc(NC(=O)c3sc(N4CC5CCC(C4)N5)cc3F)cc(F)c2n1] |
| 674 | 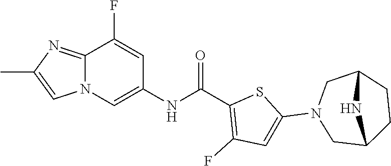 [SMILES: Cc1cn2cc(NC(=O)c3sc(N4C[C@H]5CC[C@@H](C4)N5)cc3F)cc(F)c2n1] |
| 675 | 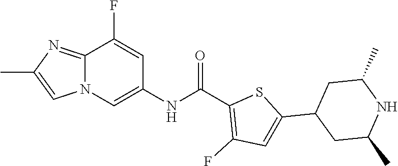 |
| 676 | 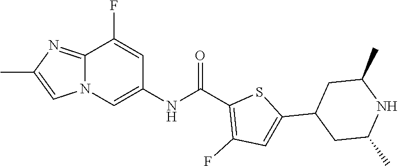 |
| 677 | 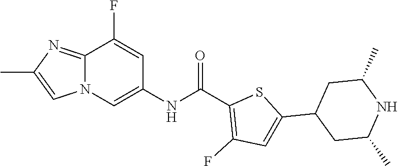 |
| 678 | 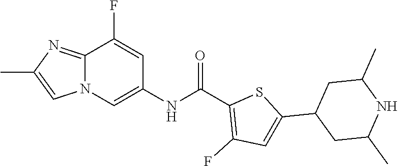 |
| 679 | 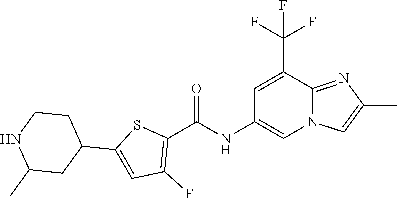 |
| 680 | 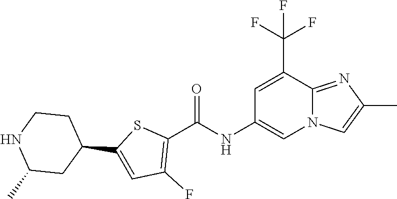 |
| 681 | 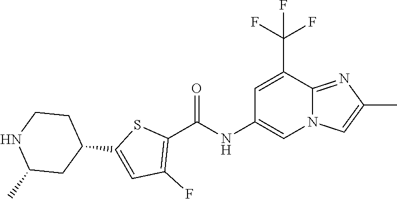 |

### Pharmaceutical Compositions, Kits, and Administration

The present invention provides pharmaceutical compositions comprising a compound of Formula (I), (II), or (III), e.g., a compound of Formula (I), (II), or (III) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (II), or (III) or a pharmaceutically acceptable salt thereof, and optionally a pharmaceutically acceptable excipient. In certain embodiments, the compound of Formula (I), (II), or (III) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (II), or (III) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension.

These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (II), or (III) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents.

Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (II), or (III) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (II), or (III) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (II), or (III). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I), (II), or (III). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), (II), or (III), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAMJ5, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNVA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCSIL, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf11, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMKID, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENP1, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CMIP, CMYA5, CNGA3, CNOT1, CNOT7, CNTN6, COG3, COLI1A1, COLI1A2, COL12A1, COL14A1, COL15A1, COL17AM, COL19A1, COL1A1, COL1A2, COL2A, COL3A1, COL4A1, COL4A2, COL4A5, COL4A6, COL5A2, COL6A1, COL7A, COL9A, COL9A2, COL22A1, COL24A1, COL25A1, COL29A1, COLQ, COMTDI, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXCl, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAIJ, DNAJA4, DNAJC13, DNAJC7, DNMTl, DNTTIP2, DOCK4, DOCK5, DOCK10, DOCK11, DOTIL, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBNl, FBX015, FBX018, FBXO38, FCGBP, FECH, FEZ2, FGA, FGD6, FGFR2, FGFRIOP, FGFRIOP2, FGFR2, FGG, FGR, FIX, FKBP3, FLI1, FLJ35848, FLJ36070, FLNA, EN1, FNBP1L, FOLH1, FOSL1, FOSL2, FOXKJ, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GF11, GFM, GH, GHR, GHV, GJA1, GLA, GLT8D1, GNA11, GNAQ, GNAS, GNB5, GOLGB1, GOLTIA, GOLTIB, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXCO, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, ILIR2, IL5RA, IL7RA, IMAIT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIHI, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIFS, KIF16B, KIFSA, KIFSB, KIF9, KIN, KIR2DL5B, KIR3DL2, KIR3DL3, KIT, KLF3, KLFS, KLF7, KLFO, KLF12, KLF16, KLHL20, KLK12, KLKB, KMT2A, KMT2B, KPNAS, KRAS, KREMEN1, KRIT1, KRTS, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCHI, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCHS, MATN2, MBD3, MCF2L2, MCM6, MDGA2, MDM4, ASXLl, FUS, SPR54, MECOM, MEF2C, MEF2D, MEGF0, MEGF1, MEMO1, MET, MGA, MGAM, MGAT4A, MGATS, MGC16169, MGC34774, MKKS, MIB1, MIER2, MITF, MKL2, MLANA, MLH1, MLLS, MLX MAlE, MPDZ, MP1, MRAP2, MRPLII, MRPL39, MRPS28, MRPS35, MS4A13, MSH2, MSH3, MSMB, MST1R, MTDH, MTERF3, MTF1, MTF2, MTHF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOAJ, NDC80, NDFIP2, NEB, NEDD4, NEK1, NEKS, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRCS, NLRP3, NLRP7, NLRP8, NLRP13, NME1, NME1-NME2, NME2, NME7, NOLI0, NOP561, NOS1, NOS2A, NOTCH1, NPAS4, NPM1, NRID1, NRIH3, NR1H4, NR4A3, NRSAI, NRXNI, NSMAF, NSMCE2, NTSC, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPAJ, OPN4, OPTN, OSBPLI1, OSBPL8, OSGEPLI, OTC, OTX2, OVOL2, OXT, PA2G4, PADI4, PAH, PAN2, PAOX, PAPOLG, PARD3, PARPI, PARVB, PAWR, PAX3, PAX8, PBGD, PBRM1, PBX2, PCBP4, PCCA, PCGF2, PCNX, PCOTH, PDCD4, PDE4D, PDE8B, PDE1OA, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5KIA, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPP1R12A, PPP3CB, PPP4C, PPP4RIL, PPP4R2, PRAME, PRC1, PRDM1, PREY1, PREX2, PRIM1, PRIM2, PRKARIA, PRKCA, PRKGI, PRMT7, PROC, PROCR, PROSC, PRODH, PROX1, PRPF40B, PRPF4B, PRRG2, PRUNE2, PSD3, PSEN1, PSMAL, PTCHI, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RABI1FIP2, RAB23, RAF1, RALBP1, RALGDS, RBICC1, RBL2, RBM39, RBM45, RBPJ, RBSN, REC8, RELB, RFC4, RFTJ, RFTNI, RHOA, RHPN2, RIF1, RIT1, RLN3, RMND5B, RNFI1, RNF32, RNFT1, RNGTT, ROCK1, ROCK2, RORA, RP1, RP6KA3, RP11-265F1, RP13-36C9, RPAP3, RPNI, RPGR, RPL22, RPL22L1, RPS6KA6, RREBI, RRMI, RRPIB, RSK2, RTEL1, RTF1, RUFY1, RUNX1, RUNX2, RXRA, RYR3, SAALI, SAE1, SALL4, SAT1, SATB2, SBCAD, SCN1A, SCN2A, SCN3A, SCN4A, SCN5A, SCN8A, SCNA, SCN11A, SCO1, SCYL3, SDCJ, SDK1, SDK2, SEC24A, SEC24D, SEC31A, SELIL, SENP3, SENP6, SENP7, SERPINA1, SETD3, SETD4, SETDB1, SEZ6, SFRS12, SGCE, SGOL2, SGPLI, SH2D1A, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX SMS, SMTN, SNCAIP, SNORD86, SNRK, SNRP70, SNX5, SNX6, SOD1, SODIO, SOS, SOS2, SOX5, SOX6, SOX8, SP1, SP2, SP3, SP110, SPAG9, SPATA13, SPATA4, SPATS1, SPECCIL, SPDEF, SP11, SPINK5, SPP2, SPTAI, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBPJ, SUCLG2, SULF2, SUPT6H, SUPT16H, SV2C, SYCP2, SYT6, SYCP1, SYTL3, SYTL5, TAF2, TARDBP, TBCID3G, TBCID8B, TBCID26, TBCID29, TBCEL, TBKI, TBP, TBPLI, TBRI, TBX, TCEB3, TCF3, TCF4, TCF7L2, TCFL5, TCF12, TCPIIL2, TDRD3, TEAD1, TEAD3, TEAD4, TECTB, TEK, TERF1, TERF2, TET2, TFAP2A, TFAP2B, TFAP2C, TFAP4, TFDPI, TFRC, TG, TGM7, TGSI, THAP7, THAP12, THOC2, TIAL1, TIAM2, TIMM50, TLK2, TM4SF20, TM6SF1, TMEM27, TMEM77, TMEM156, TMEM194A, TMFI, TMPRSS6, TNFRSF10A, TNFRSF10B, TNFRSF8, TNK2, TNKS, TNKS2, TOMIL, TOMIL2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTFI, TTLL5, TTLL9, TTN, TTPAL, TTR, TUSC3, TXNDC10, UBE3A, UCK1, UGTIA1, UHRFIBP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VTIA, VTIIB, VWA3B, WDFY2, WDR16, WDR17, WDR26, WDR44, WDR67, WDTC1, WRN, WRNIP1, WT1, WWC3, XBP1, XRN1, XRN2, XX—FW88277, YAP1, YARS, YBXJ, YGM, YY1, ZBTB18, ZBTB20, ZC3HAV1, ZC3HC1, ZC3H7A, ZDHHC19, ZEB1, ZEB2, ZFPMJ, ZFYVE1, ZFX ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, ACO000120.3, KRIT1, AC004076.1, ZNF772, AC004076.9, ZNF772, AC004223.3, RAD51D, AC004381.6, AC006486.1, ERF, AC0007390.5, AC0007780.1, PRKARIA, AC0007998.2, INO80C, AC0009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDDIA, AC073610.3, ARF3, AC074091.1,GPN, AC079447.1, LIPT, AC092587.1, AC079594.2, TRIM59, AC091060.1,C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2,RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKTIS1, AKT2, AL139011.2, PEX19, AL157935.2, ST6GALNAC6, AL358113.1,TJP2, AL441992.2, KYAT1, AL449266.1,CLCC1, AL590556.3, LINC00339, CDC42, ALAS1, ALB, ALDH16A1, ALDHIBI, ALDH3A1, ALDH3B2, ALDOA, ALKBH2, ALPL, AMD1, AMICA1, AMN1, AMOTL2, AMYJB, AMY2B, ANAPC10, ANAPC11, ANAPC15, ANG, RNASE4, AL163636.2, ANGEL2, ANGPTLI, ANKMYl, ANKRD11, ANKRD28, ANKRD46, ANKRD9, ANKS3, ANKS3,RP11-127I20.7, ANKS6, ANKZFJ, ANPEP, ANXA11, ANXA2, ANXA8L2, AL603965.1, AOC3, AP000304.12, CRYZL, AP000311.1, CRYZL, AP000893.2,RAB30, AP001267.5, ATP5MG, AP002495.2, AP003175.1, OR2AT4, AP003419.1, CLCFJ, AP005263.1, ANKRD12, AP006621.5, AP006621.1, AP1G1, AP3M1, AP3M2, APBA2, APBB1, APLP2, APOA2, APOL1, APOL3, APTX, ARAPI,STARDIO, ARF4, ARFIP1, ARFIP2, ARFRPI, ARHGAPIIA, ARHGAP33, ARHGAP4, ARHGEFIO, ARHGEF3, ARHGEF35, OR2A1-AS1, ARHGEF35, OR2A1-AS1, ARHGEF34P, ARIDIB, ARHGEF35, OR2A20P, OR2A1-AS1, ARHGEF9, ARLI, ARL13B, ARL16, ARL6, ARMC6, ARMC8, ARMCX2, ARMCX5, RP4-769N13.6, ARMCX5-GPRASP2, BHLHB9, ARMCX5-GPRASP2,GPRASP1, ARMCX5-GPRASP2,GPRASP2, ARMCX6, ARNT2, ARPP19, ARRB2, ARSA, ART3, ASB3,GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATPSF1A, ATP5G2, ATPSJ, ATPSMD, ATPSPF, ATP6AP2, ATP6VOB, ATP6V1C1, ATP6VID, ATP7B, ATXN1, ATXA1L,IST1, ATXA3, ATXA7L1, AURKA, AURKB, AXDNDl, B3GALNT1, B3GALT5, AF064860.1, B3GALT5,AF064860.5, B3GNT5, B4GALT3, B4GALT4, B9D1, BACH1, BAIAP2, BANF1, BANF2, BAX, BAZ2A, BBIPI, BCHE, BCL2L14, BCL6, BCL9L, BCSIL, BDH1, BDKRB2,AL355102.2, BEST1, BEST3, BEX4, BHLHB9, BID, BIN3, BIRC2, BIVM, BIVM-ERCC5, BIVM, BLCAP, BLK, BLOCISI, RP11-644F5.10, BLOCIS6, AC090527.2, BLOCIS6, RPII-96020.4, BLVRA, BMF, BOLA1, BORCS8-MEF2B, BORCS8, BRCA1, BRD1, BRDT, BRINP3, BROX BTBDIO, BTBD3, BTBD9, BTD, BTF3L4, BTNL9, BUB1B-PAK6, PAK6, BUB3, COorf68, C11orf1, C11orf48, C11orf54, C11orf54,AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, CID, C1GALT1, CIQB, CIQTNFI, CIS, C1orf101, C1orf112, C1orf116, C1orf159, C1orf63, C2, C2,CFB, C20orf27, C21orf58, C2CD4D, C2orf15, LIPT1, MRPL30, C2orf80, C2orf81, C3orf14, C3orf17, C3orf18, C3orf22, C3orf33,AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3,C8orf44, C8orf59, C9,DAB2, C9orf153, C9orf9, CA5BP1,CASB, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSPI, CARNS1, CASCI, CASP3, CASP7, CBFA2T2, CBS, CBYJ, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNEI, CCHCRI, CCL28, CCNB1IP1, CCNC, CCND3, CCNG1, CCP110, CCR9, CCT7, CCT8, CD151, CDID, CD200, CD22, CD226, CD276, CD36, CD59, CDC26, CDC42, CDC42SE1, CDC42SE2, CDHR3, CDK10, CDK16, CDK4, CDKAL1, CDKL3,CTD-2410N18.4, CDKNIA, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHDIL, CHD8, CHFR,ZNF605, CHIA, CHID1, CHL1, CHM, CHMPIA, CHMP3, RNF103-CHMP3, CHRNA2, CIDEC, CIRBP, CITED1, CKLF-CMTM1, CMTM1, CKMT1B, CLDN12,CTB-13L3.1, CLDND1,AC021660.3, CLDND1,CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMIJ, BMIJ, COPS5, COPS7B, COQ8A, CORO6, COTLI, COX14,RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPTIC, CREB3L2, CREM, CRP, CRYZ, CS,AC073896.1, CS, RP11-977G19.10, CSAD, CSDE, CSF2RA, CSGALNACT, CSK, CSNK2A1, CSRNP2, CT45A4, CT45A4,CT45A5, CT45A6, CTBP2, CTCFL, CTD-2116N17.1, KIAA0101, CTD-2349B8.1, SYT17, CTD-2528L19.4, ZNF607, CTD-2619J3.8, ZNF497, CTNNA1, CTNNBIP, CTNND1, CTPS2, CTSB, CTSL, CTTN, CUL2, CUL9, CWC15, CXorf40B, CYB561A3, CYBCl, CYLD, CYPI1A1, CYP2R1, CYP4B1, CYP4F22, DAG1, DAGLB,KDELR2, DARS, DBNL, DCAF11, DCAF8,PEX19, DCLREIC, DCTD, DCTN1, DCTN4, DCUN1D2, DDR1, DDX11, DDX19B, AC012184.2, DDX19B, RP11-529K.3, DDX25, DDX39B, ATP6V1G2-DDX39B, SNORD84, DDX42, DDX60L, DEDD, DEDD2, DEFA1, DEFA1B, DEFA1B, DEFA3, DENNDIC, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKNV, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRKJA, DYRK2, DYRK4, RPL1-500M8.7, DZIPIL, E2F6, ECHDC1, ECSIT, ECT2, EDC3, EDEM1, EDEM2, MA&P24-AS1, RP4-61404.11, EEFIAKNMT, EEFID, EFEMP1, EFHC1, EGFL7, EHF, E124, EIFIAD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL, ELOVL7, ELP1, ELP6, EML3, EMP3, ENC1, ENDOV, ENO1, ENPP5, ENTHD2, ENTPD6, EP400NL, EPB41L1, EPDR1,NME8, EPHXJ, EPM2A, EPN1, EPN2, EPN3, EPS8L2, ERBB3, ERC1, ERCC1, ERG, ERI2, ERI2, DCUN1D3, ERLIN2, ERMARD, ERRFIJ, ESR2,RP11-544I20.2, ESRRA, ESRRB, ESRRG, ETFA, ETFRF1, ETV1, ETV4, ETV7, EVAIA, EVC2, EVXJ, EXD2, EXO5, EXOC1, EXOC2, FAAP24, FABP6, FADS1, FADS2, FAHD2B, FAM107B, FAM111A, FAM11B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXPJ, FOXKJ, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT1l, GALNT14, GALNT6, GAPVD1, GARNL3, GAS2L3, GAS8, GATA1, GATA2, GATA4, GBA, GCNT1, GDPD2, GDPD5, GEMIN7,MARK4, GEMIN8, GGA3, GGACT, AL356966.1, GGPS1, GHRL, GID8, GIGYF2, GIMAP8, GIPC1, GJB1, GJB6, GLBIL, GLI1, GLT8D1, GMFG, GMPR2, GNA12, GNAQ, GNB1, GNB2, GNE, GNG2, GNGT2, GNPDA1, GNPDA2, GOLGA3,CHFR, GOLGA4, GOLPH3L, GOLTIB, GPBPL, GPER, GPR16, GPR141,EPDR1, GPR155, GPR161, GPR56, GPR63, GPR75-ASB3,ASB3, GPR85, GPSM2, GRAMD1B, GRB10, GRB7, GREM2, GRIA2, GSDMB, GSE1, GSN, GSTA4, GSTZ1, GTDCJ, GTF2H1, GTF2H4, VARS2, GTF3C2, GUCY1A3, GUCYIB3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1, OR51B5, HBG2,HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX HOXA3, HOXB3, HOXB3,HOXB4, HOXC4, HOXD3, HOXD3,HOXD4, HPCAL1, HPS4, HPS5, HRHJ, HS3ST3A1, HSH2D, HSP90AA1, HSPD1, HTT, HUWE1, HYOU1, IAHJ, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, ILIRAP, IL1RL1, IL18R1, ILIRN, IL32, IL4I1,NUP62,AC011452.1, IL4I1,NUP62,CTC-326K19.6, IL6ST, ILVBL, IMALPIL, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGBIBP1, ITGB7, ITIH4, RP5-966M1.6, ITPRIPL1, JADE1, JAK2, JARID2, JDP2, KANK1, KANK1,RP11-31F19.1, KANK2, KANSLIL, KAT6A, KBTBD2, KBTBD3, KCNAB2, KCNE3, KCNG1, KCNJ16, KCNJ9, KCNMB2,AC117457.1,LINC01014, KCTD20, KCTD7,RABGEF1, KDMIB, KDM4A,AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KJFC3, KIN, KIRREL1, KITLG, KLCJ, APOPT1, AL139300.1, KLC4, KLHDC4, KLHDC8A, KLHL13, KLHL18, KLHL2, KLHL24, KLHL7, KLKI1, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCHI, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MADIL1, MADIL1, AC069288.1, MAEA, MAFF, MAFG, MAFK, MAGEA12,CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3,SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGFJO, ME11, MEIS2, MELK, MET, METTL13, METTL23, MFF, MFN2, MFSD2A, MGST3, MIB2, MICAL1, MICAL3, MICOSO, NBL1,MICOS10-NBL1, MID1, MINA, MINOS1-NBL1,MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33,BABAM2, MRPL33, BRE, MRPL47, MRPL48, MRPL55, MRRF, MRTFA, MRTFB, MRV11, MS4A1, MS4A15, MS4A3, MS4A6E,MS4A7,MS4A14, MSANTD3, MSANTD4, MSH5,MSH5-SAPCD1, MSL2, MSRB3, MSS51, MTCP1,CMC4, MTERF, MTERF1, MTERF3, MTERFD2, MTERFD3, MTF2, MTG2, MTHFD2, MTHFD2L, MTHF2, MTHF3, MTMR10, MTRF1, MTRR, MTUS2, MUTYH, MVK, MX1, MX2, MYH10, MYL12A, MYB, MYD88, MYL5, MYLIP, MYNN, MYO15A, MYOIB, MYOM2, MZFJ, N4BP2L2, NAA60, NAB1, NAE1, NAGK, NAP1L1, NAPIL4, NAPG, NARFL, NARG2, NAT1, NAT10, NBPF11, W12-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1,NME1-NME2,NME2, NME1-NME2, NME2, NME4, NME6, NME9, NOD1, NOLO, NOL8, NONO, NPAS1, NPIPA8, RPII-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RPII-197N18.2, OLA1, OPRL1, OPTN, OR2H1, ORA12, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH1, PARVG, PASK, PAX6, PBRM, PBXIP1, PCBP3, PCBP4,AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIKIL, PDLIM7, PDP1, PDPKJ, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDBJ, PHOSPHO1, PHOSPHO2, KLHL23, P14 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4,FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POL1, POLL, POLRIB, POM121, POM121C,AC006014.7, POM121C, AC211429.1, POMC, POMT1, POP1, PORCN, POU5F1, PSORSIC3, PPARD, PPARG, PPHLN, PPIL3, PPIL4, PPMIA, PPM1B, AC013717.1, PPPICB, PPPIRII, PPPIR13L, PPPIR26, PPPIR9A, PPP2R2B, PPP3CA, PPP6R1, PPP6R3, PPT2,PPT2-EGFL8, EGFL8, PPWDJ, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5,PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXA, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RA114, RA12, RALBP1, RAN, RANGAP1, RAP1A, RAP1B, RAPIGAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7,AP002373.1, RBM7, RP11-212D19.4, RBMS2, RBMYIE, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4,ANG, AL163636.6, RNASEK, RNASEK-C17orf49, RNFI1, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8,CDK11A, RP11-120M18.2,PRKARIA, RP11-133K10.2, PAK6, RP11-164J13.1,CAPN3, RP11-21JJ8.J, ANKRD12, RP11-322E10.6,INO80C, RP11-337C18.10,CHDIL, RP11-432B6.3, TRIM59, RP11-468E2.4,IRF9, RP11-484M3.5, UPKIB, RP11-517H2.6, CCR6, RPII-613M10.9, SLC25A51, RP11-659G9.3, RAB30, RP11-691N7.6,CTNND1, RP11-849H4.2, RP11-896J10.3, NKX2-1, RP11-96020.4,SQRDL, RP11-986E7.7, SERPINA3, RP4-769N13.6, GPRASP1, RP4-769N13.6,GPRASP2, RP4-798P15.3, SEC16B, RP5-10211I20.4, ZNF410, RP6-109B7.3, FLJ27365, RPE, RPH3AL, RPL15, RPL17, RPL17-C18orf32,RPL17, RPL23A, RPL36,HSD11BIL, RPP38, RPS20, RPS27A, RPS3A, RPS6KA3, RPS6KC1, RPS6KL1, RPUSD1, RRAGD, RRAS2, RRBP1, RSLID1, RSRC2, RSRP1, RUBCNL, RUNXIT1, RUVBL2, RWDD1, RWDD4, SOOA13,AL162258.1, S100A13,RP1-178F15.5, S100A16, S100A4, S100A3, S100A6, SOOPBP, SAA1, SACMIL, SAMD4B, SARIA, SARAF, SARNP, RP11-762I7.5, SCAMP5, SCAP, SCAPER, SCFD1, SCGB3A2, SCIN, SCML1, SCNNID, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3,C8orf44, SH2B1, SH2D6, SH3BP1,Z83844.3, SH3BP2, SH3BP5, SH3D19, SH3YL1, SHC1, SHISA5, SHMT1, SHMT2, SHOC2, SHROOM1, SIGLEC5,SIGLEC14, SIL1, SIN3A, SIRT2, SIRT6, SKP1, STAT4, AC104109.3, SLAIN1, SLC10A3, SLC12A9, SLC14A1, SLC16A6, SLCIA2, SLCIA6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCOIA2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2,CCDC169-SOHLH2,CCDC169, SORBS1, SORBS2, SOX5, SP2, SPART, SPATA20, SPATA21, SPATS2, SPATS2L, SPDYE2, SPECC1, SPECCIL, SPECCIL-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX21P, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2AIL, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULTIA1, SULTIA2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAF1C, TAF6,AC073842.2, TAF6, RP11-506M12.1, TAF9, TAGLN, TANK, TAPSAR1, PSMB9, TAPT1, TATDN1, TAZ, TBC1D1, TBCID12, HELLS, TBCID15, TBCID3H, TBCID3G, TBCID5, TBCID5,SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCPIOL, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1,TF, TFP1, TGIF1, THAP6, THBS3, THOCS, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199,CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RPII-691N7.6,CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIP1, TNK2, TNNT, TNRC18, TNS3, TOB2, TOMIL1, TOPIMT, TOP3B, TOX2, TP53,RP11-199F1.2, TP53I1, TP53INP2, TPCN, TPM3P9,AC022137.3, TPT1, TRA2B, TRAF2, TRAF3, TRAPPC12, TRAPPC3, TREH, TREX, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1,AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGEA, XAGE1B, XKR9, XPNPEP1, XRCC3, XRN2, XXYL T1, YIFIA, YIF1B, YIPF1, YIPF5, YPEL5, YWHAB, YWHAZ, YYJAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2,PEG3, ZKSCAN1, ZKSCAN3, ZKSCAN8, ZMAT3, ZMAT5, ZMIZ2, ZMYM6, ZMYND11, ZNFO, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28,ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC0008770.4, ZNF438, ZNF444, ZNF445, ZNF467, ZNF480, ZNF493, ZNF493,CTD-2561J22.3, ZNF502, ZNF507, ZNF512, AC074091.1, ZNF512,RP11-158I13.2, ZNF512B, ZNF512B, SAMD10, ZNF521, ZNF532, ZNF544, AC020915.5, ZNF544, CTD-3138B18.4, ZNF559,ZNF177, ZNF562, ZNF567, ZNF569, ZNF570, ZNF571-AS1,ZNF540, ZNF577, ZNF580,ZNF581, ZNF580, ZNF581,CCDC106, ZNF600, ZNF611, ZNF613, ZNF615, ZNF619,ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOAM1 gene.

Exemplary genes that may be modulated by the compounds of Formula (I), (II), or (III) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgegegeg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacace (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUgucegcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I), (II), or (III) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPAO, HNRNPAI, HNRNPAILI, HNRNPAIL2, HNRNPA3, HNRNPA2B1, HNRNPAB, HNRNPBI, HNRNPC, HNRNPCLI, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (II), or (III) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (II), or (III) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (II), or (III) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), or (III) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (II), or (III). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), (II), or (III) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I), (II), or (III) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I), (II), or (III) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (II), or (III), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (II), or (III), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I), (II), or (III) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I), (II), or (III) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (II), or (III) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (II), or (III), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (II), or (III) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (II), or (III) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (II), or (III) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (II), or (III) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (II), or (III) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (II), or (III) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (II), or (III) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (II), or (III) increases expression of the haploinsufficient gene locus. In an embodiment, a compound of Formula (I), (II), or (III) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (II), or (III), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (II), or (III) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (II), or (III), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (II), or (III) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (II), or (III), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (II), or (III) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I), (II), or (III) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell permeability measurement (Papp) of <2×10⁻⁶ cm s⁻¹. In an embodiment, a compound of Formula (I), (II), or (III) or a pharmaceutically acceptable salt thereof, e.g., as described herein, has a cell permeability measurement (Papp) of between 2-6×10⁻⁶ cm s⁻¹. In an embodiment, a compound of Formula (I), (II), or (III) or a pharmaceutically acceptable salt thereof, e.g., as described herein, has a cell permeability measurement (Papp) of Papp greater than 6×10⁻⁶ cm s⁻¹. In an embodiment, a compound of Formula (I), (II), or (III) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I), (II), or (III) or a pharmaceutically acceptable salt thereof, e.g., as described herein, has a cell efflux ratio of less than 1.5. In an embodiment, a compound of Formula (I), (II), or (III) or a pharmaceutically acceptable salt thereof, e.g., as described herein, has a cell efflux ratio of between 1.5 and 5. In an embodiment, a compound of Formula (I), (II), or (III) or a pharmaceutically acceptable salt thereof, e.g., as described herein, has a cell efflux ratio greater than 5. In an embodiment, a compound of Formula (I), (II), or (III) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the expression of a target protein (e.g., HTT or MYB) in a reference cell or sample. In an embodiment, a compound of Formula (I), (II), or (III) or a pharmaceutically acceptable salt thereof, e.g., as described herein, increases the expression of a target protein (e.g., HTT or MYB) in a reference cell or sample. In an embodiment, a compound of Formula (I), (II), or (III) or a pharmaceutically acceptable salt thereof, e.g., as described herein, decreases the expression of a target protein (e.g., HTT or MYB) in a reference cell or sample. The effect of an exemplary compound of Formula (I), (II), or (III) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I), (II), or (III) or a pharmaceutically acceptable salt thereof, e.g., as described herein, has a protein abundance response less than 100 nM. In an embodiment, a compound of Formula (I), (II), or (III) or a pharmaceutically acceptable salt thereof, e.g., as described herein, has a protein abundance response between 100-1000 nM. In an embodiment, a compound of Formula (I), (II), or (III) or a pharmaceutically acceptable salt thereof, e.g., as described herein, has a protein abundance response greater than 1000 nM. In an embodiment, a compound of Formula (I), (II), or (III) or a pharmaceutically acceptable salt thereof, e.g., as described herein, has a protein abundance response greater than 10 uM. In an embodiment, a compound of Formula (I), (II), or (III) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, modulates the viability of a target cell in a subject or sample. In an embodiment, a compound of Formula (I), (II), or (III) or a pharmaceutically acceptable salt thereof, e.g., as described herein, increases the viability of a target cell in a subject or sample. In an embodiment, a compound of Formula (I), (II), or (III) or a pharmaceutically acceptable salt thereof, e.g., as described herein, decreases the viability of a target cell in a subject or sample. In an embodiment, a compound of Formula (I), (II), or (III) or a pharmaceutically acceptable salt thereof, e.g., as described herein, does not impact the viability of a cell (e.g., is non-toxic) in a subject or sample. The effect an exemplary compound of Formula (I), (II), or (III) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I), (II), or (III) or a pharmaceutically acceptable salt thereof, e.g., as described herein, is tolerated by a target cell at a concentration of less than 100 nM. In an embodiment, a compound of Formula (I), (II), or (III) or a pharmaceutically acceptable salt thereof, e.g., as described herein, is tolerated by a target cell at a concentration of between 100-1000 nM. In an embodiment, a compound of Formula (I), (II), or (III) or a pharmaceutically acceptable salt thereof, e.g., as described herein, is tolerated by a target cell at a concentration of greater than 1000 nM. In an embodiment, a compound of Formula (I), (II), or (III) or a pharmaceutically acceptable salt thereof, e.g., as described herein, is tolerated by a target cell at a concentration of greater than 10 uM.

In some embodiments, a compound of Formula (I), (II), or (III) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I), (II), or (III) or a pharmaceutically acceptable salt thereof, e.g., as described herein, has a Kp value of greater than 5. In an embodiment, a compound of Formula (I), (II), or (III) or a pharmaceutically acceptable salt thereof, e.g., as described herein, has a Kp value between 1 and 5. In an embodiment, a compound of Formula (I), (II), or (III) or a pharmaceutically acceptable salt thereof, e.g., as described herein, has a Kp value between 0.2-1. In an embodiment, a compound of Formula (I), (II), or (III) or a pharmaceutically acceptable salt thereof, e.g., as described herein, has a Kp value of less than 0.2. In an embodiment, a compound of Formula (I), (II), or (III) or a pharmaceutically acceptable salt thereof, e.g., as described herein, has a Kpuu value of greater than 2.5. In an embodiment, a compound of Formula (I), (II), or (III) or a pharmaceutically acceptable salt thereof, e.g., as described herein, has a Kpuu value between 0.5-2.5. In an embodiment, a compound of Formula (I), (II), or (III) or a pharmaceutically acceptable salt thereof, e.g., as described herein, has a Kpuu value between 0.1-0.5. In an embodiment, a compound of Formula (I), (II), or (III) or a pharmaceutically acceptable salt thereof, e.g., as described herein, has a Kpuu value of less than 0.1. In an embodiment, a compound of Formula (I), (II), or (III) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I), (II), or (III) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I), (II), or (III) or a pharmaceutically acceptable salt thereof, e.g., as described herein, exhibits selectivity for HTT, e.g., an HTT-related nucleic acid sequence. In an embodiment, a compound of Formula (I), (II), or (III) or a pharmaceutically acceptable salt thereof, e.g., as described herein, exhibits selectivity for SMN2, e.g., an SMN2-related nucleic acid sequence. In an embodiment, a compound of Formula (I), (II), or (III) or a pharmaceutically acceptable salt thereof, e.g., as described herein, exhibits selectivity for Target C, e.g., a Target C-related nucleic acid sequence. In an embodiment, a compound of Formula (I), (II), or (III) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I), (II), or (III) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I), (II), or (III) or a pharmaceutically acceptable salt thereof, e.g., as described herein, has a 3-fold greater selectivity for HTT over MYB. In an embodiment, a compound of Formula (I), (II), or (III) or a pharmaceutically acceptable salt thereof, e.g., as described herein, has a 3-fold greater selectivity for MYB over HTT. In an embodiment, a compound of Formula (I), (II), or (III) or a pharmaceutically acceptable salt thereof, e.g., as described herein, has a 10-fold greater selectivity for HTT over MYB. In an embodiment, a compound of Formula (I), (II), or (III) or a pharmaceutically acceptable salt thereof, e.g., as described herein, has a 10-fold greater selectivity for MYB over HTT. In an embodiment, a compound of Formula (I), (II), or (III) or a pharmaceutically acceptable salt thereof, e.g., as described herein, has a 3-fold greater selectivity for HTT over SMN2. In an embodiment, a compound of Formula (I), (II), or (III) or a pharmaceutically acceptable salt thereof, e.g., as described herein, has a 3-fold greater selectivity for SMN2 over HTT. In an embodiment, a compound of Formula (I), (II), or (III) or a pharmaceutically acceptable salt thereof, e.g., as described herein, has a 10-fold greater selectivity for HTT over SMN2. In an embodiment, a compound of Formula (I), (II), or (III) or a pharmaceutically acceptable salt thereof, e.g., as described herein, has a 10-fold greater selectivity for SMN2 over HTT. In an embodiment, a compound of Formula (I), (II), or (III) or a pharmaceutically acceptable salt thereof, e.g., as described herein, has a 3-fold greater selectivity for MYB over SMN2. In an embodiment, a compound of Formula (I), (II), or (III) or a pharmaceutically acceptable salt thereof, e.g., as described herein, has a 3-fold greater selectivity for SMN2 over MYB. In an embodiment, a compound of Formula (I), (II), or (III) or a pharmaceutically acceptable salt thereof, e.g., as described herein, has a 10-fold greater selectivity for MYB over SMN2. In an embodiment, a compound of Formula (I), (II), or (III) or a pharmaceutically acceptable salt thereof, e.g., as described herein, has a 10-fold greater selectivity for SMN2 over MYB. In an embodiment, a compound of Formula (I), (II), or (III) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), (II), or (III), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 m, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DFA):EtOH_1=50:50).

Preparative HPLC purification: prep-HPLC purification was performed using one of the following HPLC conditions:

Condition 1: Shimadzu, Column: XBridge Prep OBD C18 Column, 30 Å—150 mm 5 μm; Mobile Phase A: water (10 mmol/L NH₄HCO₃) Mobile Phase B: acetonitrile; Flow rate:60 mL/min; Gradient 1: 3 B to 3 B in 2 min; Gradient 2: 5% B to 35% B in 6 min; Gradient 3: 3 B to 33 B in 6 min; Gradient 4: 5% B up to 45% in 6 min; Gradient 5: 3% B to 23% B in 6 min; Gradient 6: 10% B to 60% B in 8 min; Gradient 7: 5 B to 45 B in 10 min; Gradient 8: 10% B up to 47% B in 10 min; Gradient 9: 10% B up to 50% B in 8 min; Gradient 9: 5% B to 35% B in 8 min; Gradient 10: 10% B to 48% B in 10 min; Gradient 11:20% B to 52% B in 8 min; Gradient 12: 20% B to 50% B in 6 min; Gradient 13: 20% B to 43% B in 8 min; Gradient 14: 15% B to 45% B in 8 min; Gradient 14: 10% B to 55% B in 8 min; Gradient 15: 5% B to 38% B in 10 min; Gradient 16: 10% B to 35% B in 8 min; Gradient 17: 5% B to 42% B in 8 min; Gradient 18: 5% B to 30% B in 8 min; Gradient 18: 5% B to 40% B in 8 min; Gradient 19: 5% B to 45% B in 8 min; Gradient 21: 5% B to 37% B in 8 min; Gradient 22: 5% B to 65% B in 8 min; Gradient 23: 10% B to 48% B in 6 min.

Condition 2: Column: Xselect CSH OBD Column 30*150 mm 5 μm, n; Mobile Phase A: water (10 mmol/L NH₄HCO₃); Mobile Phase B: acetonitrile; Flow rate: 60 mL/min; Gradient 1: 10 B to 55 B in 8 min; Gradient 2: 5 B to 50 B in 8 min; Gradient 3: 10 B to 60 B in 10 min; Gradient 4: 10 B to 40 B in 8 min; Gradient 5: 5 B to 65 B in 8 min; Gradient 6: 3% B to 63% B in 6 min; Gradient 7: 10% B to 52% B in 8 min; Gradient 8: 5% B to 37% B in 8 min; Gradient 9: 10% B to 38% B in 8 min; Gradient 10: 3% B to 75% B in 8 min; Gradient 11: 10% B to 42% B in 8 min; Gradient 12: 15% B to 40% B in 10 min; Gradient 13: 10% B to 60% B in 8 min; Gradient 14: 5% B to 35% B in 8 min.

Condition 3: Column: EP-C18M 10 μm 120A; Mobile Phase A: water (1 mmol/L HCl); Mobile Phase B: acetonitrile; Flow rate:100 mL/min; Gradient: 40% B to 70% B in 35 min.

Condition 4: Column: Poroshell HPH-C18, 3.0*50 mm, 2.7 um; Mobile Phase A: water (5 mM NH₄HCO₃); Mobile Phase B: acetonitrile; Flow rate: 1.2 mL/min; Gradient 1:10% B to 95% B in 1.2 min, hold 0.5 min.

Condition 5: Column: X Select CSH OBD 30×150 mm 5 μm; Mobile phase A: water (0.1% formic acid); Mobile phase B: acetonitrile; Gradient 1: 3% phase B up to 18% in 6 min.

Condition 6: Column: X Select CSH OBD 30×150 mm 5 μm; Mobile phase A: water (0.05% HCl); Mobile phase B: acetonitrile; Flow rate: 60 mL/min; Gradient 1: 3% phase B up to 3% in 2 min.

Condition 7: Column: X Select CSH OBD 30×150 mm 5 μm; Mobile phase A: water (0.05% formic acid); Mobile phase B: acetonitrile; Flow rate: 60 mL/min; Gradient 1: 3% phase B up to 20% in 8 min.

Condition 8: Column: YMC-Actus Triart C18, 30 mm×150 mm, 5 μm; Mobile phase A: water (0.05% HCl); Mobile phase B: acetonitrile; Gradient 1: 5% B to 35% B in 8 min; Gradient 2: 25% B to 85% B in 8 min.

Condition 9: Column: YMC-Actus Triart C18, 30 mm×150 mm, 5 μm; Mobile phase A: water (10 mmol/L NH₄HCO₃); Mobile phase B: acetonitrile; Flow rate: 60 mL/min Gradient 1: 10% B to 70% B in 8 min; Gradient 2: 15% B to 55% B in 8 min; Gradient 3: 5% B to 65% B in 8 min; Gradient 3: 5% B to 45% B in 8 min; Gradient 4:15% B to 45% B in 10 min.

Condition 10: Column: SunFire Prep 19*150 mm, 10 nm; Mobile Phase A: water (0.05% NH₄₀H); Mobile Phase B: acetonitrile; Gradient 1: 35% B to 55% B in 7 min; Gradient 2: 30% B to 50% B in 7 min; Gradient 3: 10% B to 20% B in 7 min.

Condition 11: Column: XBridge Prep OBD 19*150 mm, 8 μm; Mobile Phase A: water (0.05% NH₃·H₂O), Mobile Phase B: acetonitrile; Flow rate: 20 mL/min; Gradient 1: 30% B to 70% B in 8 min; Gradient 2: 10% B to 60% B in 8 min; Gradient 3: 27% B to 52% B in 8 min.

Condition 12: Column: Welch Ultimate XB-C18 50*250 mm 10 μm; Mobile Phase A: water (0.1% TFA), Mobile Phase B: acetonitrile; Flow rate: 90 mL/min; Gradient 1: 11% B to 46% B in 12 min; Gradient 2: 2% B to 30% B in 14 min; Gradient 3: 30% B to 30% B in 3 min; Gradient 4: 15% B to 60% B in 12 min.

Preparative chiral HPLC: purification by chiral HPLC was performed on a Gilson-GX 281 using column: CHIRALPAK IG-3, (CHIRALPAK IC-3 or CHIRALPAK OJ-3.

Condition 1: Column: CHIRALPAK IG, 3×25 cm, 5 μm; Mobile Phase A: MTBE (0.1% DEA), Mobile Phase B: ethanol; Flow rate:20 mL/min; Gradient 1: 50 B to 50 B in 18 min.

Condition 2: Column: CHIRAL ART Cellulose-SC, 3*25 cm, 5 m; Mobile Phase A: 0.1% 2 M NH₃ in methanol, Mobile Phase B: IPA: DCM (2: 1); Flow rate: 35 mL/min; Gradient 1: 50% B to 50% B in 18 min.

Reverse flash chromatography: purification by reverse flash chromatography was performed using one of the following conditions: Condition 1: Column, C18; Mobile phase: MeOH in water; Gradient 1, 10% to 50% in 1 0 min; Detector, UV 254 nm.

Condition 2: Column, silica gel; Mobile phase: MeOH in water; Gradient 1: 10% to 50% in 10 min; Detector, UV 254 nm.

### General Synthetic Scheme

Compounds of the present disclosure may be prepared using a synthetic protocol illustrated in the exemplary schemes (Schemes A and B) shown below.

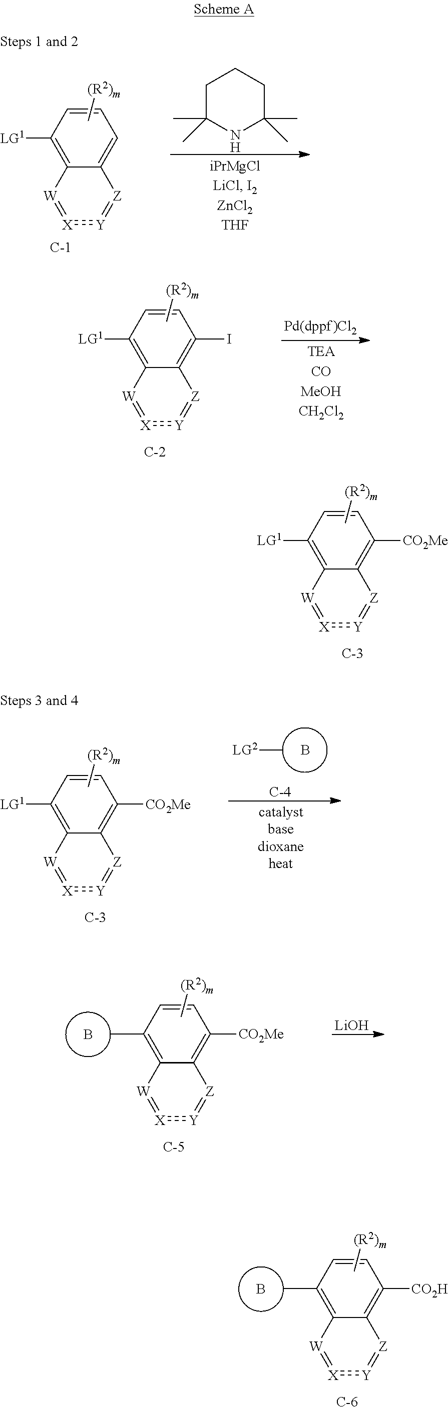

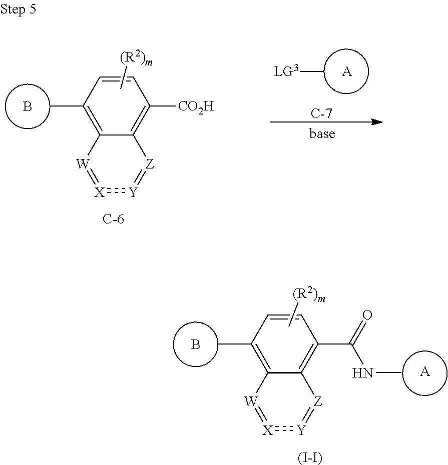

Scheme A1. An exemplary method of preparing a compound of Formula (I); wherein A, B, W, X, Y, Z, R², and m are as defined herein; and LG¹, LG², and LG³ are each independently hydrogen or a leaving group (e.g., halo, —B(OR¹²)₂).

An exemplary method of preparing a compound described herein, e.g., a compound of Formula (I-I) is provided in Scheme A1. In Step 1, C-2 is prepared by treating C-1 with a mixture of 2,2,6,6-tetramethylpiperidine, isopropylmagnesium chloride (iPrMgCl), lithium chloride (LiCl), iodine (I₂), and zinc chloride (ZnCl₂) in tetrahydrofuran (THF), or with a similar combination of reagents or solvent. In Step 2, C-3 is prepared by incubating C-2 with 1,1′-bis(diphenylphosphino)ferrocene)palladium(II) dichloride (Pd(dppf)Cl₂), carbon monoxide (CO), and triethylamine (TEA), in a mixture of methanol (MeOH) and dichloromethane (CH₂Cl₂) or a similar mixture of solvents. Alternative catalysts to Pd(dppf)Cl₂ may also be used, such as a suitable palladium catalyst, and/or using alternative reagents sufficient to provide C-3.

In Step 3, C-5 is prepared by incubating C-3 with C-4 in the presence of a catalyst, such as a ruthenium, palladium, or copper catalyst (e.g., Pd(dppf)Cl₂, Pd₂(dba)₃, XPhos-Pd(II)-G2, XPhos-Pd(II)-G3, ^{t}BuBrettphos-Pd(II)-G3, RuPhos-Pd(II), RuPhos-Pd(II)-G2, RuPhos-Pd(II)-G3, or CuI, or similar reagent), and a base (e.g., Cs₂CO₃, K₂CO₃, or a similar reagent). The reaction may be conducted in dioxane or a similar solvent, at 80° C., 100° C. or a temperature sufficient to provide C-5. C-5 is then converted to C-6 by treatment with lithium hydroxide at a temperature sufficient to provide C-6.

C-6 and C-7 are coupled to provide a compound of Formula (I-I) in Step 5. This coupling reaction may be conducted in the presence of a base (e.g., Cs₂CO₃, DIEA, N-methylimidazole, or a suitable alternative). The reaction may be conducted in dimethylformamide or a similar solvent, at a temperature sufficient to provide the compound of Formula (I-I). Each starting material and/or intermediate in Scheme A1 may be protected and deprotected using standard protecting group methods. In addition, purification and characterization of each intermediate as well as the final compound of Formula (I) may be afforded by any accepted procedure.

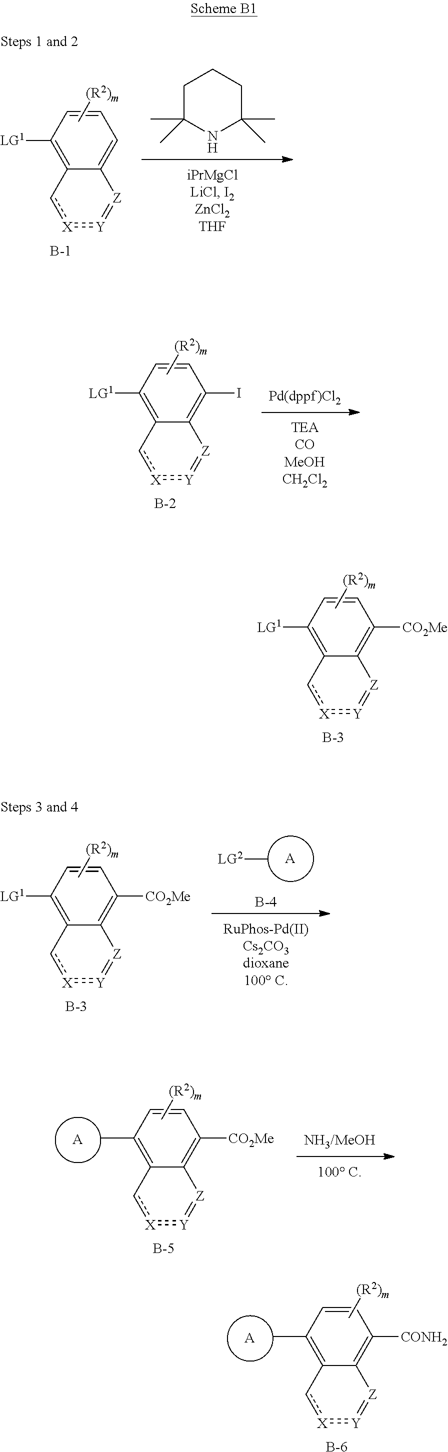

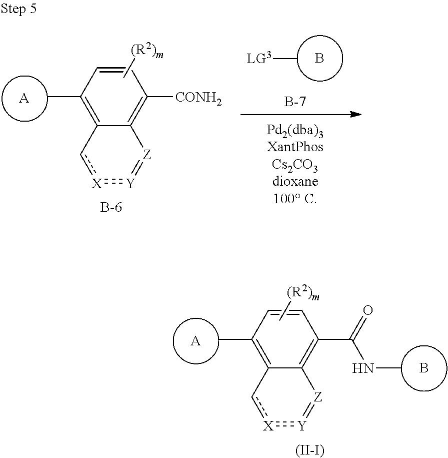

Scheme B1. An exemplary method of preparing a compound of Formula (I); wherein A, B, X, Y, Z, R², and m are as defined herein; and LG¹, LG², and LG³ are each independently a leaving group (e.g., halo, —B(OR¹²)₂).

An exemplary method of preparing a compound described herein, e.g., a compound of Formula (I-I) is provided in Scheme B1. In Step 1, B-2 is prepared by treating B-1 with a mixture of 2,2,6,6-tetramethylpiperidine, isopropylmagnesium chloride (iPrMgCl), lithium chloride (LiCl), iodine (I₂), and zinc chloride (ZnCl₂) in tetrahydrofuran (THF), or with a similar combination of reagents or solvent. In Step 2, B-3 is prepared by incubating B2 with 1,1′-bis(diphenylphosphino)ferrocene)palladium(II) dichloride (Pd(dppf)Cl₂), carbon monoxide (CO), and triethylamine (TEA), in a mixture of methanol (MeOH) and dichloromethane (CH₂Cl₂) or a similar mixture of solvents. Alternative catalysts to Pd(dppf)Cl₂ may also be used, such as a suitable palladium catalyst, and/or using alternative reagents sufficient to provide B-3.

In Step 3, B-5 is prepared by incubating B-3 with B-4 in the presence of RuPhos-Pd(II) (e.g., RuPhos-Pd(II)-G2 or RuPhos-Pd(II)-G3), and cesium carbonate (Cs₂CO₃) or a similar reagent. Step 3 may also be carried out using an alternative catalyst to RuPhos-Pd(II), such as another ruthenium catalyst. The reaction may be conducted in dioxane or a similar solvent, at 100° C. or a temperature sufficient to provide B-5. B-5 is then converted to B-6 by treatment with a mixture of ammonia and methanol, at 100° C. or a temperature sufficient to provide B-6.

B-6 and B-7 are coupled to provide a compound of Formula (I-I) in Step 5. This coupling reaction may be conducted in the presence of tris(dibenzylideneacetone)dipalladium(0) (Pd₂(dba)₃, XantPhos, and cesium carbonate or a suitable alternative. Step 5 may also be carried out using an alternative catalyst to Pd₂(dba)₃, such as another palladium catalyst, and/or an alternative ligand to XantPhos (e.g., a different phosphine ligand). The reaction may be conducted in dioxane or a similar solvent, at 100° C. or a temperature sufficient to provide the compound of Formula (I-I). Each starting material and/or intermediate in Scheme B may be protected and deprotected using standard protecting group methods. In addition, purification and characterization of each intermediate as well as the final compound of Formula (I) may be afforded by any accepted procedure.

Exemplary protocols for the synthesis of compounds in Table 1, e.g., Compounds 1-160, which represent Examples 1-56, can be found in WO 2021/174164. Exemplary protocols for the synthesis of compounds in Table 3, e.g., Compounds 575-681, can be found in WO 2021/174174. Each of the foregoing references are incorporated herein by reference in their entirety.

### Example 57: Synthesis of Compound 183

### Synthesis of Intermediate C1

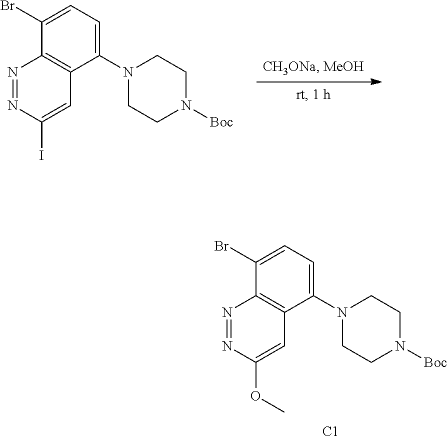

To a stirred solution of tert-butyl 4-(8-bromo-3-iodocinnolin-5-yl)piperazine-1-carboxylate (300 mg, 0.462 mmol, 1.0 equiv, 80%) in DMF (4.8 mL) was added CH₃ONa (166.5 mg, 0.924 mmol, 2.0 equiv) dropwise at room temperature. The resulting mixture was stirred for 1 h at room temperature, then diluted with water (15 mL) and extracted with ethyl acetate (3×15 mL). The organic layers were combined, washed with water (3×15 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(8-bromo-3-methoxycinnolin-5-yl)piperazine-1-carboxylate (140 mg, 72%) as a solid. LCMS (ES, m/z): 423.2 [M+H]⁺.

### Synthesis of Intermediate C2

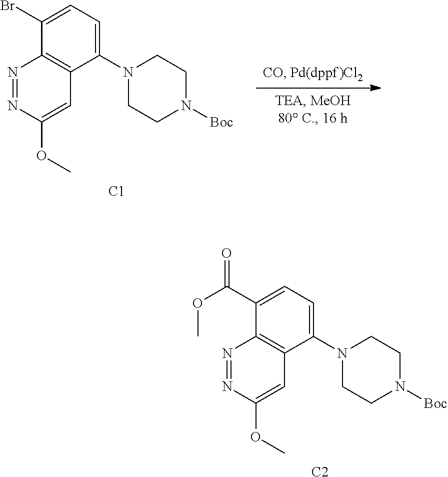

To a solution of tert-butyl 4-(8-bromo-3-methoxycinnolin-5-yl)piperazine-1-carboxylate (140 mg, 0.331 mmol, 1.0 equiv) in methanol (20 mL) was added Pd(dppf)Cl₂ (24.2 mg, 0.033 mmol, 0.1 equiv) in a pressure tank. The mixture was purged with nitrogen for 1 min, then pressurized to 1 MPa with carbon monoxide at 80° C. for 16 h. The reaction mixture was cooled to room temperature, then filtered to remove residual solids. The filtrate was concentrated under vacuum to give a residue. The residue was purified by silica gel column chromatography, eluted with PE/EA (1:1) to afford methyl 5-[4-(tert-butoxycarbonyl)piperazin-1-yl]-3-methoxycinnoline-8-carboxylate (50 mg, 38%) as a solid. LCMS (ES, m/z):403.2 [M+H]⁺.

### Synthesis of Intermediate C3

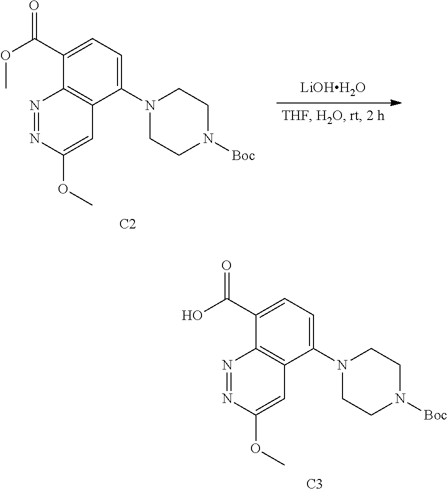

To a stirred mixture of methyl 5-[4-(tert-butoxycarbonyl)piperazin-1-yl]-3-methoxycinnoline-8-carboxylate (50 mg, 0.124 mmol, 1.0 equiv) in THF (1 mL) and water (1 mL) was added lithiumol hydrate (41.7 mg, 0.992 mmol, 8.0 equiv) at room temperature. The resulting mixture was stirred for 2 h at 50° C., then concentrated under vacuum and diluted with water (5 mL). The resulting mixture was acidified to pH 6 with concentrated HCl and extracted with ethyl acetate (3×5 mL). The organic layers were combined, dried over anhydrous Na₂SO₄, and filtered. After filtration, the filtrate was concentrated under reduced pressure to afford 5-[4-(tert-butoxycarbonyl)piperazin-1-yl]-3-methoxycinnoline-8-carboxylic acid (40 mg, 83%) as a solid. LCMS (ES, m/z): 389.0[M+H]⁺.

### Synthesis of Intermediate C4

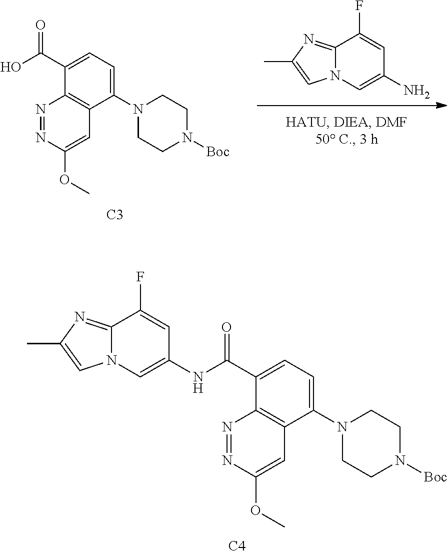

To a stirred mixture of 5-[4-(tert-butoxycarbonyl)piperazin-1-yl]-3-methoxycinnoline-8-carboxylic acid (40 mg, 0.103 mmol, 1.0 equiv) and HATU (70.5 mg, 0.185 mmol, 1.8 equiv) in DMF (1 mL) was added DIEA (39.9 mg, 0.309 mmol, 3.0 equiv) and 8-fluoro-2-methylimidazo[1,2-a]pyridin-6-amine (34.0 mg, 0.206 mmol, 2.0 equiv) at room temperature. The resulting mixture was stirred for 3 h at 50° C., then diluted with water (5 mL) and extracted with ethyl acetate (3×5 mL). The organic layers were combined, washed with water (3×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[8-({8-fluoro-2-methylimidazo[1,2-a]pyridin-6-yl}carbamoyl)-3-methoxycinnolin-5-yl]piperazine-1-carboxylate (20 mg, 36%) as a solid. LCMS (ES, m/z):536.3 [M+H]⁺.

### Synthesis of Compound 183

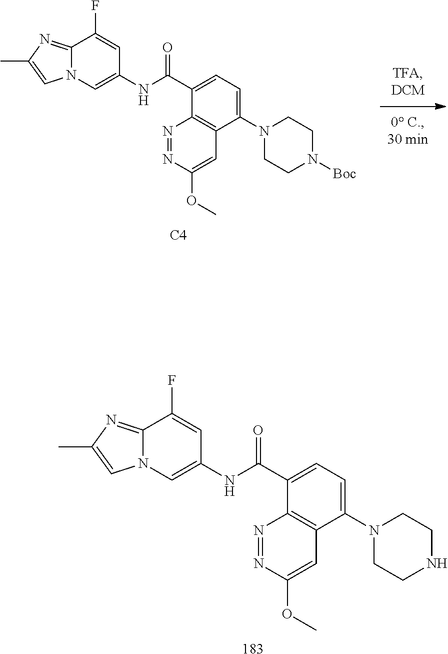 [SMILES: COc1cc2c(N3CCN(C(=O)OC(C)(C)C)CC3)ccc(C(=O)Nc3cc(F)c4nc(C)cn4c3)c2nn1 ; "C4" ; COc1cc2c(N3CCNCC3)ccc(C(=O)Nc3cc(F)c4nc(C)cn4c3)c2nn1 ; "183"]

To a stirred solution of tert-butyl 4-[8-({8-fluoro-2-methylimidazo[1,2-a]pyridin-6-yl}carbamoyl)-3-methoxycinnolin-5-yl]piperazine-1-carboxylate (25 mg, 0.047 mmol, 1.0 equiv) in DCM (1 mL) was added TFA (0.3 mg) dropwise at 0° C. The resulting mixture was stirred for 0.5 h at 0° C., then concentrated under vacuum to give a residue. The residue was purified by Prep-HPLC (Condition 1, Gradient 1) to afford N-{8-fluoro-2-methylimidazo[1,2-a]pyridin-6-yl}-3-methoxy-5-(piperazin-1-yl)cinnoline-8-carboxamide (5.1 mg, 25%) as a solid. LCMS (ES, m/z):436.2 [M+H]⁺. ¹H NMR (400 MHz, DMSO-d6) δ 11.95 (s, 1H), 9.20 (d, J=1.6 Hz, 1H), 8.29 (d, J=7.8 Hz, 1H), 7.94 (d, J=3.1 Hz, 1H), 7.64 (s, 1H), 7.36-7.28 (m, 2H), 4.26 (s, 3H), 3.09-3.02 (m, 4H), 3.02-2.96 (m, 4H), 2.36 (s, 3H).

### Example 58: Synthesis of Compound 162

### Synthesis of Intermediate C5

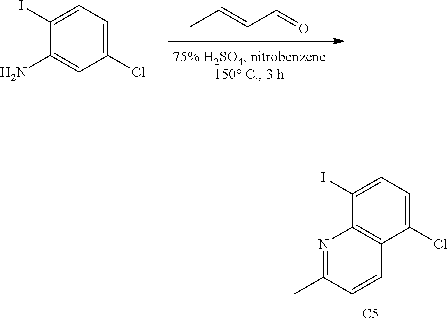

To a stirred mixture of 5-chloro-2-iodoaniline (10.0 g, 39.4 mmol, 1.0 equiv), crotonaldehyde (3.3 g, 47.3 mmol, 1.2 equiv), and nitrobenzene (4 mL) was added 75% H₂SO₄ (7 mL) at room temperature. The reaction mixture was stirred for 3 h at 150° C. under nitrogen atmosphere, then cooled to room temperature. The resulting mixture was diluted with water (20 mL), basified to pH 7 with saturated Na₂CO₃ (aq.), and extracted with ethyl acetate (3×50 mL). The organic layers were combined, washed with brine (1×50 mL), dried over anhydrous Na₂SO₄, and filtered. After filtration, the filtrate was concentrated under reduced pressure to give a residue. The residue was purified by silica gel column chromatography, eluted with PE/EA (5:1) to afford 5-chloro-8-iodo-2-methylquinoline (2 g, 17%) as a solid. LCMS (ES, m/z): 304 [M+H]⁺. ¹H NMR (400 MHz, DMSO-d₆) δ 8.43 (d, J=8.6 Hz, 1H), 8.34 (d, J=8.0 Hz, 1H), 7.65 (d, J=8.6 Hz, 1H), 7.51 (d, J=8.0 Hz, 1H), 2.76 (s, 3H).

### Synthesis of Intermediate C6

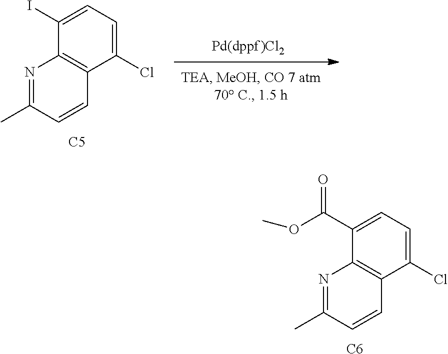

To a stirred mixture of 5-chloro-8-iodo-2-methylquinoline (2 g, 6.5 mmol, 1.0 equiv), methanol (20 mL), and TEA (2.0 g, 19.7 mmol, 3.0 equiv) was added Pd(dppf)Cl₂ (480 mg, 0.6 mmol, 0.1 equiv) at room temperature. The reaction mixture was stirred for 1.5 h at 70° C. under CO atmosphere, then cooled to room temperature and concentrated under reduced pressure to give a residue. The residue was purified by silica gel column chromatography, eluted with PE/EA (3:1) to afford methyl 5-chloro-2-methylquinoline-8-carboxylate (1.3 g, 84%) as a solid. LCMS (ES, m/z): 236 [M+H]⁺. ¹H NMR (300 MHz, DMSO-d₆) δ 8.51 (d, J=8.7 Hz, 1H), 7.89 (d, J=7.8 Hz, 1H), 7.78 (d, J=7.8 Hz, 1H), 7.66 (d, J=8.7 Hz, 1H), 3.92 (s, 3H), 2.69 (s, 3H).

### Synthesis of Intermediate C7

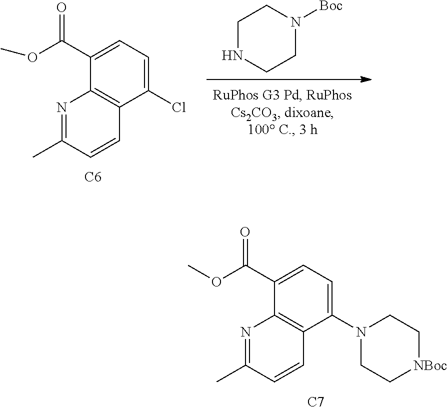

A mixture of methyl 5-chloro-2-methylquinoline-8-carboxylate (300 mg, 1.2 mmol, 1.0 equiv), tert-butyl piperazine-1-carboxylate (474.1 mg, 2.5 mmol, 2 equiv), Cs₂CO₃ (1244.2 mg, 3.8 mmol, 3 equiv), RuPhos (118.8 mg, 0.2 mmol, 0.2 equiv), RuPhos Pd G3 (0.1 equiv) and dioxane (3 mL) was stirred for 3 h at 100° C. under nitrogen atmosphere, then cooled to room temperature. The resulting mixture was diluted with water (5 mL) and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford methyl 5-[4-(tert-butoxycarbonyl)piperazin-1-yl]-2-methylquinoline-8-carboxylate) as a solid. LCMS (ES, m/z): 386 [M+H]⁺. ¹H NMR (300 MHz, DMSO-d₆) δ 8.42 (d, J=8.7 Hz, 1H), 7.82 (d, J=7.9 Hz, 1H), 7.46 (d, J=8.7 Hz, 1H), 7.12 (d, J=7.9 Hz, 1H), 3.87 (s, 3H), 3.61 (s, 4H), 3.02 (s, 4H), 2.64 (s, 3H), 1.45 (s, 9H).

### Synthesis of Intermediate C8

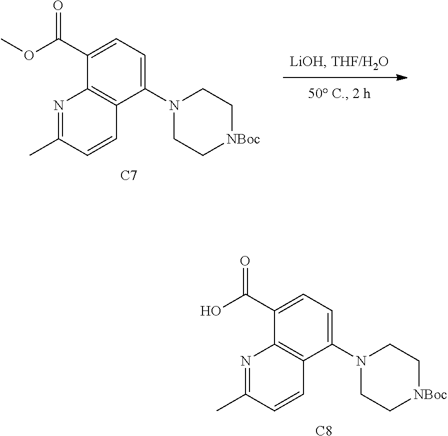

To a stirred mixture of methyl 5-[4-(tert-butoxycarbonyl)piperazin-1-yl]-2-methylquinoline-8-carboxylate (350 mg, 0.9 mmol, 1.0 equiv) and LiOH·H₂O (152.4 mg, 3.6 mmol, 4 equiv) was added THF (4 mL) and water (4 mL). The reaction mixture was stirred for 2 h at 50° C., then cooled to room temperature. The resulting mixture was diluted with water (5 mL), acidified to pH 6 with 1 N of HCl (aq.), and extracted with ethyl acetate (3×10 mL). The organic layers were combined, washed with brine (1×10 mL), dried over anhydrous Na₂SO₄, and filtered. After filtration, the filtrate was concentrated under reduced pressure to afford 5-[4-(tert-butoxycarbonyl)piperazin-1-yl]-2-methylquinoline-8-carboxylic acid (320 mg, 95%) as a solid. LCMS (ES, m/z): 372 [M+H]⁺. ¹H NMR (300 MHz, DMSO-d₆) δ 8.64 (d, J=8.8 Hz, 1H), 8.48 (d, J=8.2 Hz, 1H), 7.65 (d, J=8.7 Hz, 1H), 7.30 (d, J=8.2 Hz, 1H), 3.62 (d, J=5.5 Hz, 4H), 3.12 (t, J=4.9 Hz, 4H), 2.80 (s, 3H), 1.45 (s, 9H).

### Synthesis of Intermediate C9

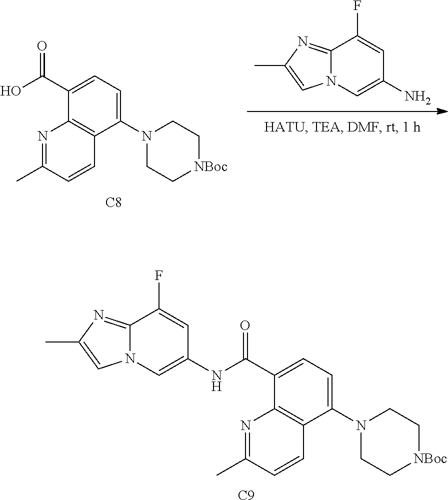

To a stirred mixture of 5-[4-(tert-butoxycarbonyl)piperazin-1-yl]-2-methylquinoline-8-carboxylic acid (80 mg, 0.2 mmol, 1.0 equiv) and 8-fluoro-2-methylimidazo[1,2-a]pyridin-6-amine (39.1 mg, 0.2 mmol, 1.1 equiv) in DMF (2 mL) was added HATU (122.8 mg, 0.3 mmol, 1.5 equiv) and TEA (65.3 mg, 0.6 mmol, 3 equiv) at room temperature. The reaction mixture was stirred for 1 h at room temperature, then quenched with water (5 mL) at room temperature and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[8-({8-fluoro-2-methylimidazo[1,2-a]pyridin-6-yl}carbamoyl)-2-methylquinolin-5-yl]piperazine-1-carboxylate (70 mg, 63%) as a solid. LCMS (ES, m/z): 519 [M+H]⁺.

### Synthesis of Compound 162

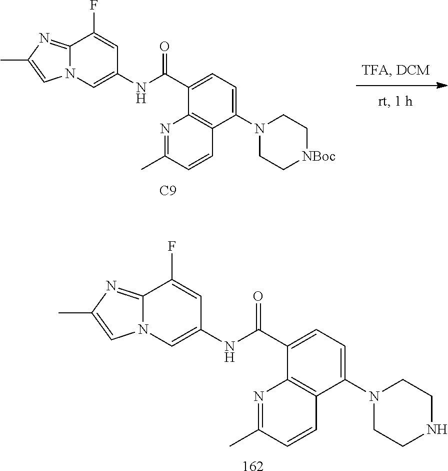

To a stirred mixture of tert-butyl 4-[8-({8-fluoro-2-methylimidazo[1,2-a]pyridin-6-yl}carbamoyl)-2-methylquinolin-5-yl]piperazine-1-carboxylate (80 mg, 0.1 mmol, 1.0 equiv) in DCM (0.8 mL) was added TFA (0.16 mL). The reaction mixture was stirred for 1 h at room temperature, then concentrated under reduced pressure to give a residue. The residue was purified by Prep-HPLC (Condition 2, Gradient 1) to afford N-{8-fluoro-2-methylimidazo[1,2-a]pyridin-6-yl}-2-methyl-5-(piperazin-1-yl)quinoline-8-carboxam-ide, trifluoroacetic acid (35.8 mg, 55%) as a solid. LCMS (ES, m/z): 419 [M+H]⁺. 1H NMR (400 MHz, DMSO-d₆) δ 13.85 (s, 1H), 9.42 (s, 1H), 8.94 (s, 2H), 8.63 (dd, J=8.4, 4.8 Hz, 2H), 8.09 (s, 1H), 7.61 (dd, J=18.0, 10.3 Hz, 2H), 7.39 (d, J=8.2 Hz, 1H), 3.44 (s, 4H), 3.33 (s, 4H), 2.92 (s, 3H), 2.43 (s, 3H).

### Example 59: Synthesis of Compound 167

### Synthesis of Intermediate C10

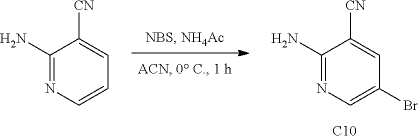 [SMILES: N#Cc1cccnc1N ; N#Cc1cc(Br)cnc1N ; "C10"]

To a stirred solution of 2-aminopyridine-3-carbonitrile (5 g, 41.97 mmol, 1.0 equiv) in acetonitrile (50 mL) was added ammonium acetate (0.3 g, 4.15 mmol, 0.1 equiv) and NBS (8.2 g, 46.16 mmol, 1.1 equiv) at 0° C. under. The resulting mixture was stirred for 1 h at 0° C., then diluted with water and extracted with ethyl acetate (3×50 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (1:1) to afford 2-amino-5-bromopyridine-3-carbonitrile (7.2 g, 87%) as a solid. LCMS (ES, m/z): 198 [M+H]⁺.

### Synthesis of Intermediate C11

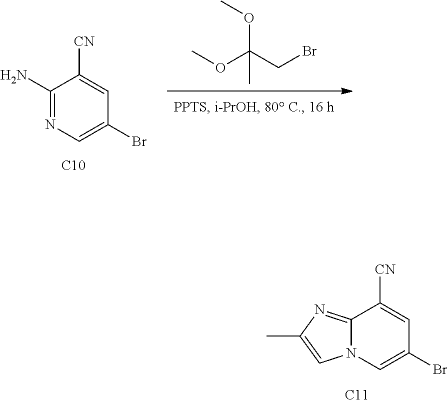

To a stirred mixture of 2-amino-5-bromopyridine-3-carbonitrile (2 g, 10.10 mmol, 1.0 equiv) and 1-bromo-2,2-dimethoxypropane (2.0 g, 11.11 mmol, 1.1 equiv) in i-PrOH (20 mL) was added PPTS (250 mg, 1.01 mmol, 0.1 equiv) at room temperature. The resulting mixture was stirred for 16 h at 80° C., then cooled to room temperature. The resulting mixture was diluted with water (30 mL) and extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (2:1) to afford 6-bromo-2-methylimidazo[1,2-a]pyridine-8-carbonitrile (2.2 g, 92%) as a solid. LCMS (ES, m/z): 236 [M+H]⁺.

### Synthesis of Intermediate C12

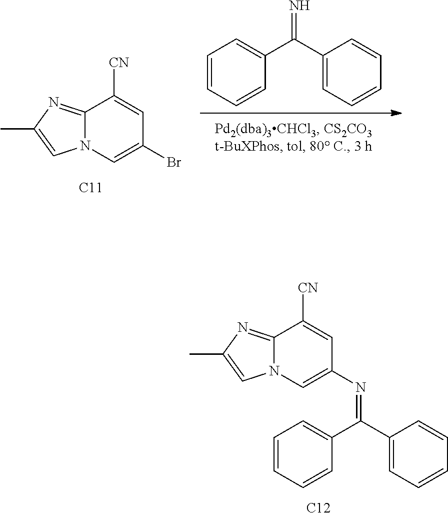 [SMILES: Cc1cn2cc(Br)cc(C#N)c2n1 ; N=C(c1ccccc1)c1ccccc1 ; "C11" ; Cc1cn2cc(N=C(c3ccccc3)c3ccccc3)cc(C#N)c2n1 ; "C12"]

To a stirred mixture of 6-bromo-2-methylimidazo[1,2-a]pyridine-8-carbonitrile (0.8 g, 3.38 mmol, 1.0 equiv) and diphenylmethanimine (0.6 g, 3.38 mmol, 1.0 equiv) in toluene (8 mL) was added Cs₂CO₃ (3.31 g, 10.16 mmol, 3.0 equiv), t-BuXPhos (0.29 g, 0.67 mmol, 0.2 equiv), and Pd₂(dba)₃CHCl₃ (350 mg, 0.33 mmol, 0.1 equiv) at room temperature. The resulting mixture was stirred for 3 h at 80° C. under nitrogen atmosphere, then cooled to room temperature. The resulting mixture was diluted with water (20 mL) and extracted with ethyl acetate (3×20 mL). The organic layers were combined, washed with brine (1×20 mL), dried over anhydrous Na₂SO₄, and filtered. After filtration, the filtrate was concentrated under reduced pressure to give a residue. The residue was purified by silica gel column chromatography, eluted with PE/EA (2:1) to afford 6-[(diphenylmethylidene)amino]-2-methylimidazo[1,2-a]pyridine-8-carbonitrile (700 mg, 61%) as a solid. LCMS (ES, m/z): 355 [M+H]⁺.

### Synthesis of Intermediate C13

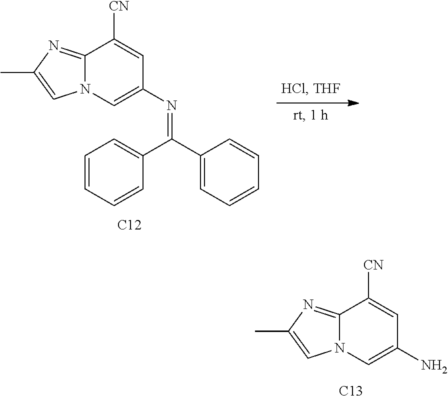

To a stirred solution of 6-[(diphenylmethylidene)amino]-2-methylimidazo[1,2-a]pyridine-8-carbonitrile (700 mg, 2.08 mmol, 1.0 equiv) in THE (3.5 mL) was added HCl (6 M) (3.5 mL) at room temperature. The resulting mixture was stirred for 1 h at room temperature, then diluted with water (10 mL), neutralized to pH 7 with saturated aqueous of Na₂CO₃, and extracted with ethyl acetate (3×10 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford 6-amino-2-methylimidazo[1,2-a]pyridine-8-carbonitrile; methylamine (320 mg, 76%) as a solid. LCMS (ES, m/z): 173 [M+H]⁺.

### Synthesis of Intermediate C14

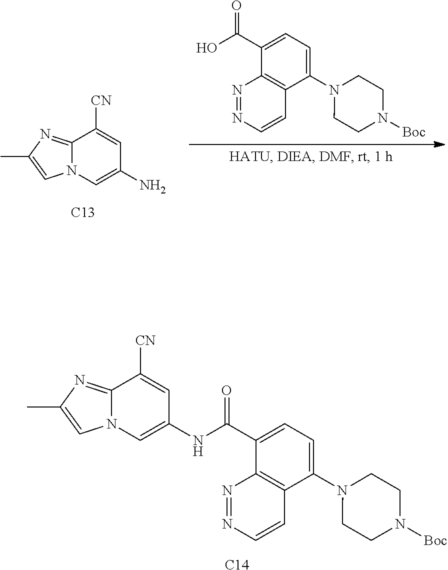 [SMILES: Cc1cn2cc(N)cc(C#N)c2n1 ; CC(C)(C)OC(=O)N1CCN(c2ccc(C(=O)O)c3nnccc23)CC1 ; "HATU, DIEA, DMF, rt, 1 h" ; "C13" ; Cc1cn2cc(NC(=O)c3ccc(N4CCN(C(=O)OC(C)(C)C)CC4)c4ccnnc34)cc(C#N)c2n1 ; "C14"]

To a stirred mixture of 6-amino-2-methylimidazo[1,2-a]pyridine-8-carbonitrile (100 mg, 0.58 mmol, 1.0 equiv) and 5-[4-(tert-butoxycarbonyl)piperazin-1-yl]cinnoline-8-carboxylic acid (312.2 mg, 0.87 mmol, 1.5 equiv) in DMF (2 mL) was added HATU (441.6 mg, 1.16 mmol, 2.0 equiv) and DIEA (225.1 mg, 1.74 mmol, 3.0 equiv) at room temperature. The resulting mixture was stirred for 1 h at room temperature, then diluted with water (2 mL) and extracted with ethyl acetate (3×3 mL). The organic layers were combined, washed with brine (1×3 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[8-({8-cyano-2-methylimidazo[1,2-a]pyridin-6-yl}carbamoyl)cinnolin-5-yl]piperazine-1-carboxylate (90 mg, 30%) as a solid. LCMS (ES, m/z): 513 [M+H]⁺.

### Synthesis of Compound 167

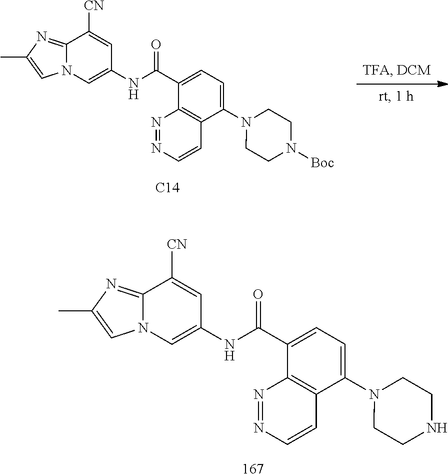

To a stirred solution of tert-butyl 4-[8-({8-cyano-2-methylimidazo[1,2-a]pyridin-6-yl}carbamoyl) cinnolin-5-yl]piperazine-1-carboxylate (90 mg, 0.17 mmol, 1.0 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 2, Gradient 2) to afford N-{8-cyano-2-methylimidazo[1,2-a]pyridin-6-yl}-5-(piperazin-1-yl)cinnoline-8-carboxamide trifluoroacetic acid salt (23.8 mg, 32.86%) as a yellow solid. LCMS (ES, m/z): 413 [M+H]⁺. ¹H NMR (400 MHz, DMSO-d₆) δ 12.21 (s, 1H), 9.64 (s, 1H), 9.57 (d, J=5.9 Hz, 1H), 8.87 (s, 2H), 8.52 (dd, J=13.0, 6.9 Hz, 2H), 8.06 (d, J=19.0 Hz, 2H), 7.60 (d, J=8.1 Hz, 1H), 3.46 (s, 4H), 3.38 (s, 4H), 2.41 (s, 3H).

### Example 60: Synthesis of Compound 165

### Synthesis of Intermediate C15

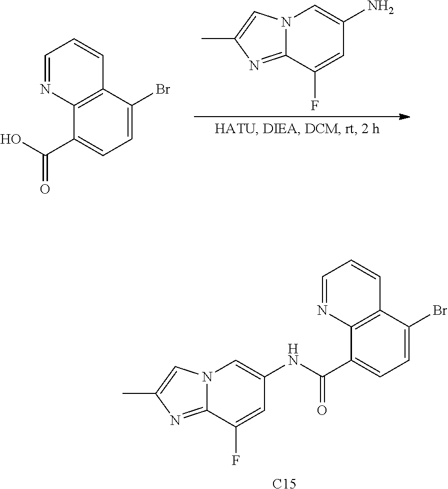

To a stirred mixture of 8-fluoro-2-methylimidazo[1,2-a]pyridin-6-amine (190 mg, 1.150 mmol, 1.00 equiv) and 5-bromoquinoline-8-carboxylic acid (318.95 mg, 1.265 mmol, 1.10 equiv) in DCM (38.00 mL) was added HATU (524.86 mg, 1.380 mmol, 1.2 equiv) and DIEA (446.01 mg, 3.450 mmol, 3 equiv) in portions at room temperature. The resulting mixture was stirred for 2 h at room temperature, then concentrated under vacuum to give a residue. The residue was purified by reverse flash chromatography (Condition 1, Gradient 1) to afford 5-bromo-N-{8-fluoro-2-methylimidazo[1,2-a] pyridin-6-yl}quinoline-8-carboxamide (245 mg, 53%) as a solid. LCMS (ES, m/z): 399 [M+H]⁺.

### Synthesis of Intermediate C16

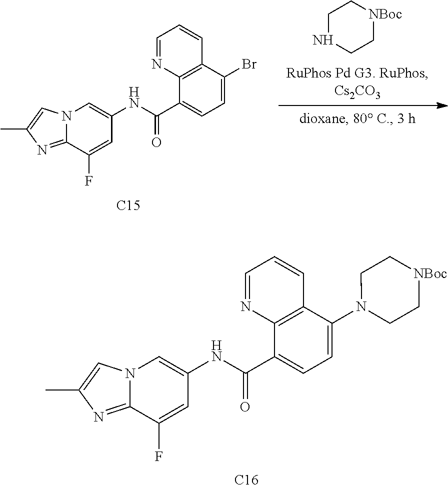

To a stirred mixture of 5-bromo-N-{8-fluoro-2-methylimidazo[1,2-a]pyridin-6-yl}quinoline-8-carboxamide (40 mg, 0.100 mmol, 1.00 equiv), Cs₂CO₃ (98.29 mg, 0.300 mmol, 3 equiv) and tert-butyl piperazine-1-carboxylate (37.32 mg, 0.200 mmol, 2 equiv) in dioxane (5 mL) was added RuPhos (9.35 mg, 0.020 mmol, 0.2 equiv) and RuPhos Palladacycle Gen.3 (8.38 mg, 0.010 mmol, 0.1 equiv) in portions at room temperature under nitrogen atmosphere. The resulting mixture was stirred for 3 h at 80° C. under nitrogen atmosphere, then cooled to room temperature. The resulting mixture was diluted with water (20 mL) and extracted with ethyl acetate (3×5 mL). The organic layers were combined, washed with brine (1×10 mL), dried over anhydrous Na₂SO₄, and filtered. After filtration, the filtrate was concentrated under reduced pressure to give a residue. The residue was purified by reverse flash chromatography (Condition 1, Gradient 2) to afford tert-butyl 4-[8-({8-fluoro-2-methylimidazo[1,2-a]pyridin-6-yl}carbamoyl) quinolin-5-yl]piperazine-1-carboxylate (40 mg, 79%) as a solid. LCMS (ES, m/z): 505 [M+H]⁺.

### Synthesis of Compound 165

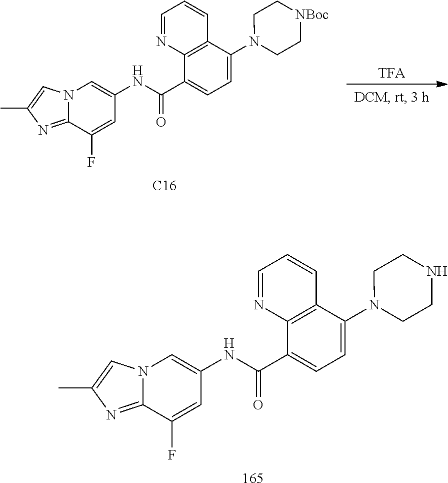 [SMILES: Cc1cn2cc(NC(=O)c3ccc(N4CCN(C(=O)OC(C)(C)C)CC4)c4cccnc34)cc(F)c2n1 ; "DCM, rt, 3 h" ; "TFA" ; "C16" ; Cc1cn2cc(NC(=O)c3ccc(N4CCNCC4)c4cccnc34)cc(F)c2n1 ; "165"]

A mixture of tert-butyl 4-[8-({8-fluoro-2-methylimidazo[1,2-a]pyridin-6-yl}carbamoyl)quinolin-5-yl]piperazine-1-carboxylate (45 mg, 0.089 mmol, 1.00 equiv) in TFA (2 mL) and DCM (2 mL) was stirred for 2 h at room temperature. The resulting mixture was concentrated under reduced pressure, then basified to pH 8 with 7 M NH₃(g) in methanol. The resulting mixture was concentrated under reduced pressure to give a residue. The residue was purified by Prep-HPLC (Condition 3, Gradient 1) to afford N-{8-fluoro-2-methylimidazo[1,2-a]pyridin-6-yl}-5-(piperazin-1-yl)quinoline-8-carboxamide (18 mg, 50%) as a solid. LCMS (ES, m/z): 405 [M+H]⁺. 1H NMR (400 MHz, DMSO-d₆) δ 13.48 (s, 1H), 9.31 (d, J=1.6 Hz, 1H), 9.18 (dd, J=4.3, 1.8 Hz, 1H), 8.67 (dd, J=8.5, 1.8 Hz, 1H), 8.60 (d, J=8.2 Hz, 1H), 7.92 (d, J=3.1 Hz, 1H), 7.73 (dd, J=8.6, 4.3 Hz, 1H), 7.49 (dd, J=12.5, 1.7 Hz, 1H), 7.33 (d, J=8.2 Hz, 1H), 3.11 −3.05 (m, 4H), 3.00 (t, J=4.4 Hz, 4H), 2.36 (s, 3H).

### Example 61: Synthesis of Compound 178

### Synthesis of Intermediate C17

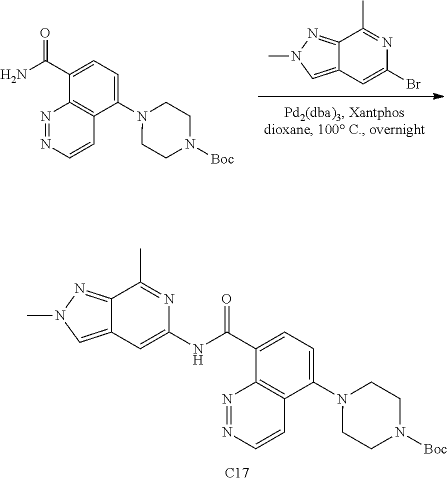 [SMILES: CC(C)(C)OC(=O)N1CCN(c2ccc(C(N)=O)c3nnccc23)CC1 ; Cc1nc(Br)cc2cn(C)nc12 ; Cc1nc(NC(=O)c2ccc(N3CCN(C(=O)OC(C)(C)C)CC3)c3ccnnc23)cc2cn(C)nc12 ; "C17"]

A mixture of tert-butyl 4-(8-carbamoylcinnolin-5-yl)piperazine-1-carboxylate (120.0 mg, 0.336 mmol, 1.0 equiv), 5-bromo-2,7-dimethylpyrazolo[3,4-c]pyridine (91.0 mg, 0.403 mmol, 1.2 equiv), Pd₂(dba)₃ (30.7 mg, 0.034 mmol, 0.1 equiv), Xantphos (38.8 mg, 0.067 mmol, 0.2 equiv), and dioxane (4 mL) was stirred overnight at 100° C. under nitrogen atmosphere. The resulting mixture was concentrated under reduced pressure to give a residue. The residue was purified by silica gel column chromatography, eluted with CH₂Cl₂/MeOH (20:1) to afford tert-butyl 4-[8-({2,7-dimethylpyrazolo[3,4-c]pyridin-5-yl}carbamoyl)cinnolin-5-yl]piperazine-1-carboxylate (120 mg, 64%) as a solid. LCMS (ES, m/z): 503 [M+H]⁺.

### Synthesis of Compound 178

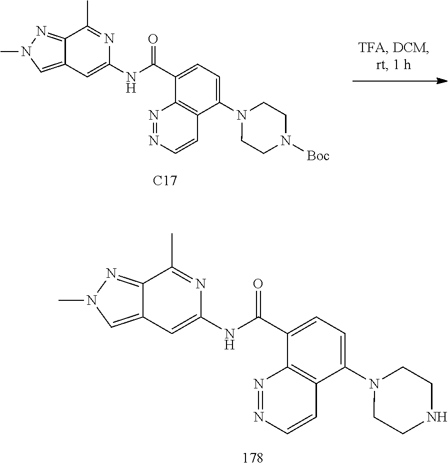 [SMILES: Cc1nc(NC(=O)c2ccc(N3CCN(C(=O)OC(C)(C)C)CC3)c3ccnnc23)cc2cn(C)nc12 ; "C17" ; Cc1nc(NC(=O)c2ccc(N3CCNCC3)c3ccnnc23)cc2cn(C)nc12 ; "178"]

A mixture of tert-butyl 4-[8-({2,7-dimethylpyrazolo[3,4-c]pyridin-5-yl}carbamoyl) cinnolin-5-yl]piperazine-1-carboxylate (90 mg, 0.179 mmol, 1.0 equiv), DCM (1 mL) and TFA (1 mL) was stirred for 1 h at room temperature. The resulting mixture was concentrated under reduced pressure to give a residue. The residue was purified by Prep-HPLC (Condition 3, Gradient 2) to afford N-{2,7-dimethylpyrazolo[3,4-c]pyridin-5-yl}-5-(piperazin-1-yl)cinnoline-8-carboxamide (30 mg, 41%) as a solid. LCMS (ES, m/z): 403 [M+H]⁺. ¹H NMR (300 MHz, DMSO-d₆) δ 13.12 (s, 1H), 9.53 (d, J=5.9 Hz, 1H), 8.76 (d, J=8.1 Hz, 1H), 8.48-8.35 (m, 3H), 7.47 (d, J=8.2 Hz, 1H), 4.24 (s, 3H), 3.17-3.07 (m, 4H), 3.00 (t, J=4.6 Hz, 4H), 2.76 (s, 3H).

### Example 63: Synthesis of Compound 170

### Synthesis of Intermediate C20

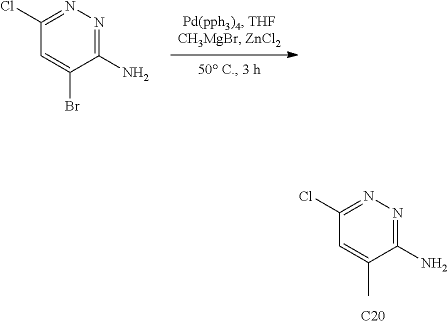

To a stirred solution of 4-bromo-6-chloropyridazin-3-amine (2 g, 9.595 mmol, 1.0 equiv) in THE (20 mL) was added CH₃MgBr (8.9 mL, 77.249 mmol, 8.0 equiv) dropwise at 0° C. under N₂ atmosphere. To the reaction mixture was added Pd(PPh₃)₄(110.9 mg, 0.096 mmol, 0.01 equiv) and ZnCl₂ (6.8 mL, 49.884 mmol, 5.2 equiv) at room temperature under N₂ atmosphere. The resulting mixture was stirred for an additional 30 min at room temperature under N₂ atmosphere. To the resulting mixture was added CH₃MgBr (8.9 mL, 77.249 mmol, 8.0 equiv) in portions at 50° C. The resulting mixture was stirred for 3 h at 50° C. under N₂ atmosphere, then diluted with water (80 mL) and extracted with ethyl acetate (3×100 mL). The organic layers were combined, washed with water (1×200 mL) and brine (1×200 mL), dried over anhydrous Na₂SO₄, and filtered. After filtration, the filtrate was concentrated under reduced pressure to give a residue. The residue was purified by silica gel column chromatography, eluted with PE/EA (1/1) to afford 6-chloro-4-methylpyridazin-3-amine (1.47 g, 99%) as a solid. LCMS (ES, m/z):144 [M+H]⁺.

### Synthesis of Intermediate C21

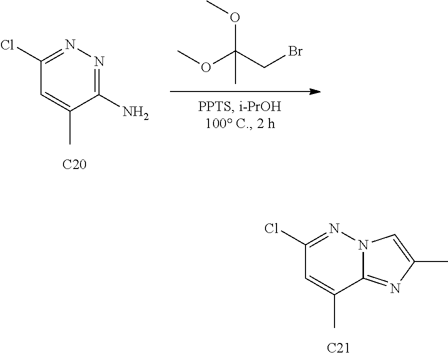

To a stirred mixture of 6-chloro-4-methylpyridazin-3-amine (1.46 g, 10.169 mmol, 1.0 equiv) and 1-bromo-2,2-dimethoxypropane (2.23 g, 12.203 mmol, 1.2 equiv) in isopropanol (29.2 mL) was added PPTS (0.18 g, 0.712 mmol, 0.07 equiv) dropwise at room temperature. The resulting mixture was stirred for 2 h at 100° C., then filtered, and the filter cake washed with isopropanol (3×50 mL). The filtrate was concentrated under reduced pressure to afford 6-chloro-2,8-dimethylimidazo[1,2-b]pyridazine (1.4 g, 76%) as a solid. LCMS (ES, m/z):182 [M+H]⁺.

### Synthesis of Intermediate C22

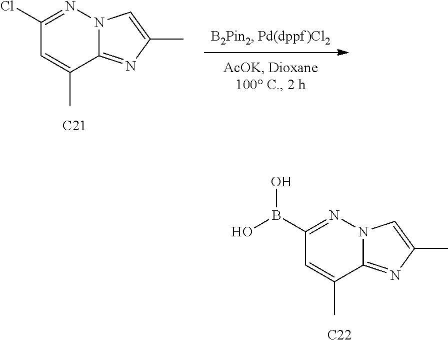

To a stirred mixture of 6-chloro-2,8-dimethylimidazo[1,2-b]pyridazine (20 mg, 0.110 mmol, 1.0 equiv) and 4,4,5,5-tetramethyl-2-(tetramethyl-1,3,2-dioxaborolan-2-yl)-1,3,2-dioxaborolane (41.9 mg, 0.165 mmol, 1.5 equiv) in dioxane (0.5 mL) was added AcOK (21.61 mg, 0.220 mmol, 2.0 equiv) and Pd(dppf)Cl₂ (40.29 mg, 0.055 mmol, 0.1 equiv) at room temperature under nitrogen atmosphere. The resulting mixture was stirred for 2 h at 100° C. under nitrogen atmosphere. LCMS (ES, m/z):192.0 [M+H]⁺.

### Synthesis of Intermediate C23

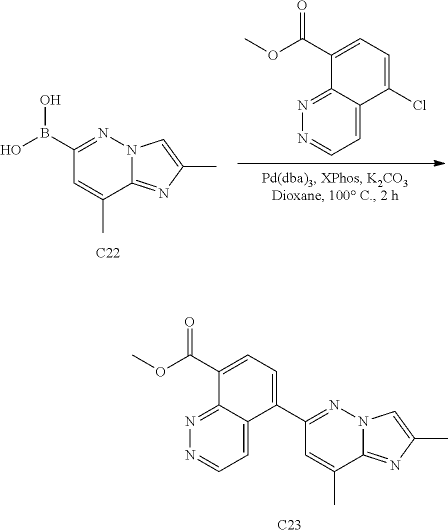

To a stirred solution of 2,8-dimethylimidazo[1,2-b]pyridazin-6-ylboronic acid (100 mg, 0.524 mmol, 1.0 equiv) in dioxane (1 mL) was added K₂CO₃ (144.7 mg, 1.048 mmol, 2.0 equiv) and Pd(dppf)Cl₂ (38.3 mg, 0.052 mmol, 0.1 equiv) at room temperature under nitrogen atmosphere. The resulting mixture was stirred for 2 h at 100° C. under nitrogen atmosphere, then concentrated under reduced pressure to give a residue. The residue was purified by Prep-TLC (CH₂Cl₂/MeOH 20:1) to afford methyl 5-{2,8-dimethylimidazo[1,2-b]pyridazin-6-yl}cinnoline-8-carboxylate (20 mg, 11%) as a solid. LCMS (ES, m/z): 334.2 [M+H]⁺.

### Synthesis of Intermediate C24

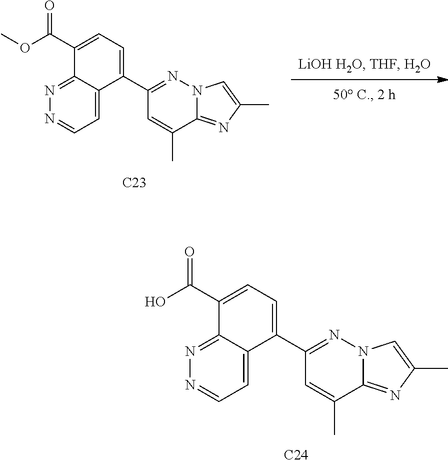

To a stirred mixture of methyl 5-{2,8-dimethylimidazo[1,2-b]pyridazin-6-yl}cinnoline-8-carboxylate (20 mg, 0.060 mmol, 1.0 equiv) in THF (0.1 mL) and water (0.1 mL) was added LiOH·H₂O (12.6 mg, 0.300 mmol, 5.0 equiv) at room temperature. The resulting mixture was stirred for 2 h at 50° C., then concentrated under vacuum. LCMS (ES, m/z):320 [M+H]⁺.

### Synthesis of Intermediate C25

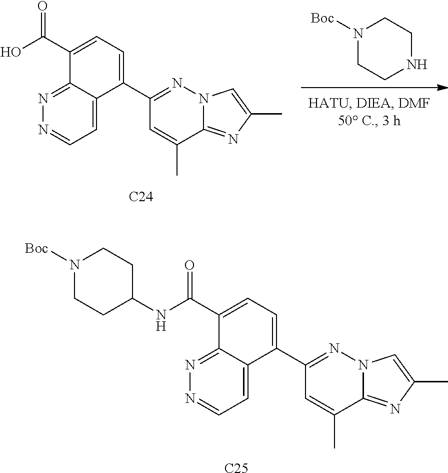 [SMILES: Cc1cn2nc(-c3ccc(C(=O)O)c4nnccc34)cc(C)c2n1 ; CC(C)(C)OC(=O)N1CCNCC1 ; "C24" ; Cc1cn2nc(-c3ccc(C(=O)NC4CCN(C(=O)OC(C)(C)C)CC4)c4nnccc34)cc(C)c2n1 ; "C25"]

To a stirred solution of 5-{2,8-dimethylimidazo[1,2-b]pyridazin-6-yl}cinnoline-8-carboxylic acid (30 mg, 0.094 mmol, 1.0 equiv) in DMF (0.6 mL) was added DIEA (36.4 mg, 0.282 mmol, 3.0 equiv), HATU (53.6 mg, 0.141 mmol, 1.5 equiv), and tert-butyl 4-aminopiperidine-1-carboxylate (24.5 mg, 0.122 mmol, 1.3 equiv) at room temperature. The resulting mixture was stirred for 3 h at 50° C., then diluted with water (3 mL) and extracted with ethyl acetate (3×3 mL). The combined organic layers were washed with water (3×3 mL), dried over anhydrous Na₂SO₄, and filtered. After filtration, the filtrate was concentrated under reduced pressure to afford tert-butyl 4-(5-{2,8-dimethylimidazo[1,2-b]pyridazin-6-yl}cinnoline-8-amido)piperidine-1-carboxylate (40 mg, 64%) as a solid. LCMS (ES, m/z):502 [M+H]⁺.

### Synthesis of Compound 170

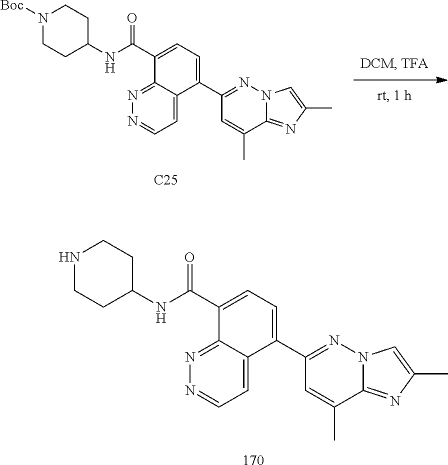 [SMILES: Cc1cn2nc(-c3ccc(C(=O)NC4CCN(C(=O)OC(C)(C)C)CC4)c4nnccc34)cc(C)c2n1 ; "C25" ; Cc1cn2nc(-c3ccc(C(=O)NC4CCNCC4)c4nnccc34)cc(C)c2n1 ; "170"]

To a stirred solution of tert-butyl 4-(5-{2,8-dimethylimidazo[1,2-b]pyridazin-6-yl}cinnoline-8-amido)piperidine-1-carboxylate (30 mg, 0.060 mmol, 1.0 equiv) in DCM (1 mL) was added TFA (0.3 mL) dropwise at 0° C. The resulting mixture was stirred for 30 min at 0° C., then concentrated under reduced pressure to give a residue. The residue was purified by Prep-HPLC (Condition 2, Gradient 3) to afford 5-{2,8-dimethylimidazo[1,2-b]pyridazin-6-yl}-N-(piperidin-4-yl)cinnoline-8-carboxamide trifluoroacetic acid salt (1.4 mg, 6%) as a solid. LCMS (ES, m/z):402 [M+H]⁺. ¹H NMR (300 MHz, Chloroform-d) δ 11.27 (d, J=8.3 Hz, 1H), 9.52 (d, J=6.1 Hz, 1H), 9.09 (d, J=7.6 Hz, 1H), 8.45 (s, 1H), 8.17 (d, J=7.5 Hz, 1H), 7.91 (s, 1H), 7.42 (s, 1H), 4.53-4.28 (m, 1H), 3.65-3.47 (m, 2H), 3.24-3.22 (m, 2H), 2.91-2.90 (m, 4H), 2.71-2.70 (m, 4H), 2.57-2.43 (m, 2H), 2.31-2.13 (m, 2H), 1.28 (s, 3H).

### Example 64: Synthesis of Compound 166

### Synthesis of Intermediate C26

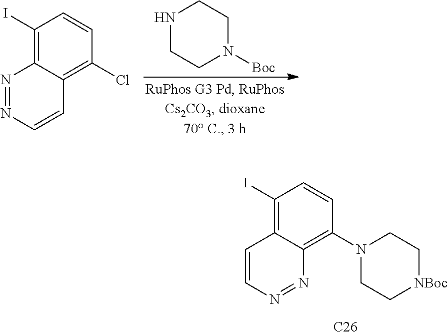 [SMILES: Clc1ccc(I)c2nnccc12 ; CC(C)(C)OC(=O)N1CCNCC1 ; CC(C)(C)OC(=O)N1CCN(c2ccc(I)c3ccnnc23)CC1 ; "C26"]

To a stirred mixture of 5-chloro-8-iodocinnoline (90.0 mg, 0.310 mmol, 1.0 equiv) and tert-butyl piperazine-1-carboxylate (69.2 mg, 0.372 mmol, 1.2 equiv) in dioxane (1 mL) was added Cs₂CO₃ (302.8 mg, 0.930 mmol, 3.0 equiv) and RuPhos Palladacycle Gen.3 (25.9 mg, 0.031 mmol, 0.1 equiv) at room temperature under nitrogen atmosphere. The resulting mixture was stirred for 3 h at 70° C. under nitrogen atmosphere. The resulting mixture was concentrated under vacuum to give a residue. The residue was purified by silica gel column chromatography, eluted with PE/EA (3:1) to afford tert-butyl 4-(5-chlorocinnolin-8-yl)piperazine-1-carboxylate (80 mg, 74%) as an oil. LCMS (ES, m/z):349 [M+H]⁺.

### Synthesis of Intermediate C27

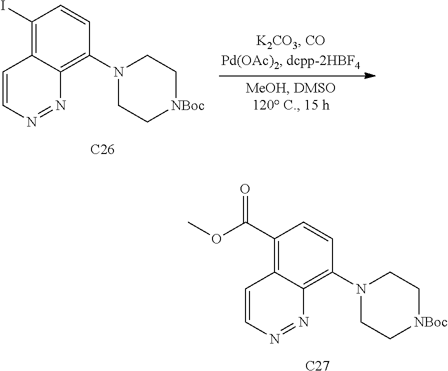 [SMILES: CC(C)(C)OC(=O)N1CCN(c2ccc(I)c3ccnnc23)CC1 ; "C26" ; COC(=O)c1ccc(N2CCN(C(=O)OC(C)(C)C)CC2)c2nnccc12 ; "C27"]

To a stirred mixture of tert-butyl 4-(5-chlorocinnolin-8-yl)piperazine-1-carboxylate (80.0 mg, 0.229 mmol, 1.0 equiv) in DMSO (5 mL) and methanol (1 mL) was added K₂CO₃ (47.5 mg, 0.344 mmol, 1.5 equiv), Pd(OAc)₂ (5.1 mg, 0.023 mmol, 0.1 equiv), and DCPP (28.1 mg, 0.046 mmol, 0.2 equiv) at room temperature under carbon dioxide atmosphere. The resulting mixture was stirred for 15 h at 120° C. under carbon dioxide atmosphere. The resulting mixture was diluted with water (20 mL) and extracted with ethyl acetate (2×20 mL). The organic layers were combined, washed with water (1×40 mL), dried over anhydrous Na₂SO₄, and filtered. After filtration, the filtrate was concentrated under reduced pressure to give a residue. The residue was purified by silica gel column chromatography, eluted with PE/EA (3/1) to afford methyl 8-(4-(tert-butoxycarbonyl)piperazin-1-yl)cinnoline-5-carboxylate (28 mg, 33%) as a solid. LCMS (ES, m/z):373 [M+H]⁺.

### Synthesis of Intermediate C28

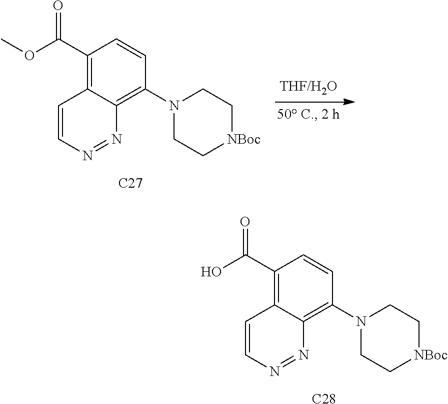 [SMILES: COC(=O)c1ccc(N2CCN(C(=O)OC(C)(C)C)CC2)c2nnccc12 ; "C27" ; CC(C)(C)OC(=O)N1CCN(c2ccc(C(=O)O)c3ccnnc23)CC1 ; "C28"]

To a stirred mixture of methyl 8-[4-(tert-butoxycarbonyl)piperazin-1-yl]cinnoline-5-carboxylate (28.0 mg, 0.075 mmol, 1.0 equiv) in THE (0.5 mL) and water (0.5 mL) was added LiOH·H₂O (9.5 mg, 0.225 mmol, 3.0 equiv) at room temperature. The resulting mixture was stirred for 2 h at 50° C., then neutralized to pH 7 with HCl (aq.) (1 N) and extracted with ethyl acetate (3×2 mL). The organic layers were combined, dried over anhydrous Na₂SO₄, and filtered. After filtration, the filtrate was concentrated under reduced pressure to give a residue. The residue was purified by silica gel column chromatography, eluted with EA to afford 8-[4-(tert-butoxycarbonyl)piperazin-1-yl]cinnoline-5-carboxylic acid (25 mg, 93%) as a solid. LCMS (ES, m/z):359 [M+H]⁺.

### Synthesis of Intermediate C29

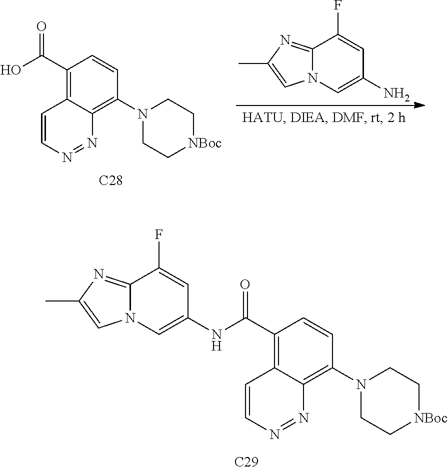 [SMILES: CC(C)(C)OC(=O)N1CCN(c2ccc(C(=O)O)c3ccnnc23)CC1 ; Cc1cn2cc(N)cc(F)c2n1 ; "C28" ; Cc1cn2cc(NC(=O)c3ccc(N4CCN(C(=O)OC(C)(C)C)CC4)c4nnccc34)cc(F)c2n1 ; "C29"]

To a stirred mixture of 8-[4-(tert-butoxycarbonyl)piperazin-1-yl]cinnoline-5-carboxylic acid (25.0 mg, 0.070 mmol, 1.0 equiv) and 8-fluoro-2-methylimidazo[1,2-a]pyridin-6-amine (11.5 mg, 0.070 mmol, 1.0 equiv) in DMF (0.5 mL) was added DIEA (22.5 mg, 0.175 mmol, 2.5 equiv) and HATU (31.8 mg, 0.084 mmol, 1.2 equiv) at room temperature. The resulting mixture was stirred for 16 h at 50° C. The resulting mixture was diluted with water (3 mL) and extracted with ethyl acetate (2×3 mL). The combined organic layers were washed with water (1×5 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[5-({8-fluoro-2-methylimidazo[1,2-a]pyridin-6-yl}carbamoyl)cinnolin-8-yl]piperazine-1-carboxylate (28 mg, 79%) as a solid.

LCMS (ES, m/z):506 [M+H]⁺.

### Synthesis of Compound 166

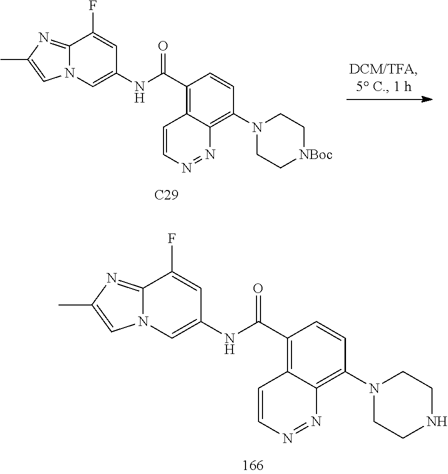 [SMILES: Cc1cn2cc(NC(=O)c3ccc(N4CCN(C(=O)OC(C)(C)C)CC4)c4nnccc34)cc(F)c2n1 ; "C29" ; Cc1cn2cc(NC(=O)c3ccc(N4CCNCC4)c4nnccc34)cc(F)c2n1]

To a stirred mixture of tert-butyl 4-(5-((8-fluoro-2-methylimidazo[1,2-a]pyridin-6-yl)carbamoyl)cinnolin-8-yl)piperazine-1-carboxylate (28.0 mg, 0.055 mmol, 1.0 equiv) in DCM (1 mL) was added TFA (0.3 mL) dropwise at 5° C. The resulting mixture was stirred for 1 h at 5° C., then concentrated under vacuum to give a residue. The residue was purified by Prep-HPLC (Condition 2, Gradient 4) to afford N-(8-fluoro-2-methylimidazo[1,2-a]pyridin-6-yl)-8-(piperazin-1-yl)cinnoline-5-carboxamide 2,2,2-trifluoroacetate (7.8 mg, 27%) as a solid. LCMS (ES, m/z):406 [M+H]⁺. ¹H NMR (300 MHz, DMSO-d₆) δ 10.76 (s, 1H), 9.45 (d, J=6.1 Hz, 1H), 9.27 (s, 1H), 8.92-8.91 (m, 2H), 8.62 (d, J=6.1 Hz, 1H), 8.20 (d, J=8.1 Hz, 1H), 8.08 (d, J=2.8 Hz, 1H), 7.63-7.28 (m, 2H), 3.85-3.84 (m, 4H), 3.46-3.45 (m, 4H), 2.40 (s, 3H).

### Example 65: Synthesis of Compound 169

### Synthesis of Intermediate C30

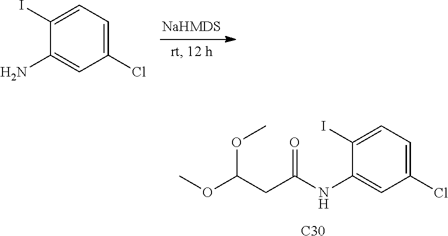

To a stirred solution of 5-chloro-2-iodoaniline (10 g, 39.452 mmol, 1.00 equiv) and methyl 3,3-dimethoxypropanoate (7 g, 47.342 mmol, 1.2 equiv) in THE (200 mL) was added NaHMDS (60 mL, 59.178 mmol, 1.5 equiv, 1 M in THF) dropwise at 0° C. The resulting mixture was stirred for 12 h at room temperature, acidified to pH 3 with citric acid solution, and extracted with ethyl acetate (3×150 mL). The organic layers were combined, washed with brine (1×50 mL), dried over anhydrous Na₂SO₄, and filtered. After filtration, the filtrate was concentrated under reduced pressure to afford N-(5-chloro-2-iodophenyl)-3,3-dimethoxypropanamide. LCMS (ES, m/z): 370 [M+H]⁺.

### Synthesis of Intermediate C31

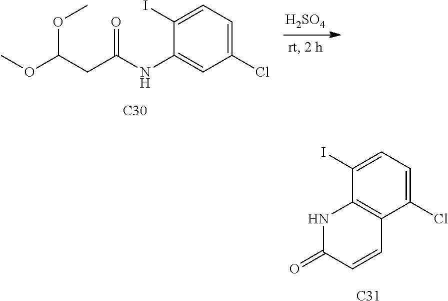

A mixture of N-(5-chloro-2-iodophenyl)-3,3-dimethoxypropanamide (15 g, 40.587 mmol, 1.00 equiv) and sulfuric acid (59.70 g, 608.805 mmol, 15 equiv) in DCM (200 mL) was stirred for 2 h at room temperature. The resulting mixture was concentrated under reduced pressure, then diluted with water (300 mL). A solid precipitated that was collected by filtration and washed with water (2×50 mL) to afford 5-chloro-8-iodoquinolin-2(1H)-one. LCMS (ES, m/z): 306 [M+H]⁺

### Synthesis of Intermediate C32

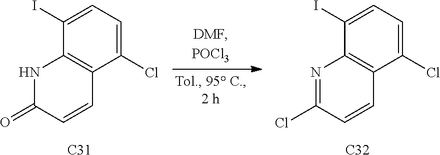 [SMILES: O=c1ccc2c(Cl)ccc(I)c2[nH]1 ; Clc1ccc2c(Cl)ccc(I)c2n1 ; "C31" ; "C32"]

To a stirred mixture of 5-chloro-8-iodo-1H-quinolin-2-one (10 g, 32.733 mmol, 1.00 equiv) and DMF (3.59 g, 49.099 mmol, 1.5 equiv) in toluene (60 mL) was added phosphorus oxychloride (4.01 g, 26.186 mmol, 0.8 equiv) at 0° C. The resulting mixture was stirred for 2 h at 95° C., then basified to pH 8 with saturated aqueous NaHCO₃, and extracted with ethyl acetate (3×50 mL). The organic layers were combined, washed with brine (1×50 mL), dried over anhydrous Na₂SO₄, and filtered. After filtration, the filtrate was concentrated under reduced pressure to give a residue. The residue was purified by silica gel column chromatography, eluted with PE:EA (9:1) to afford 2,5-dichloro-8-iodoquinoline (3.4 g, 32%) as a solid. LCMS (ES, m/z): 324 [M+H]⁺.

### Synthesis of Intermediate C33

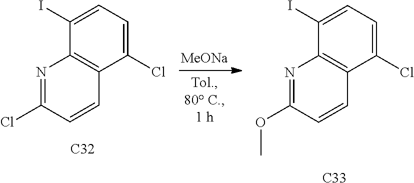

To a stirred solution of 2,5-dichloro-8-iodoquinoline (3.4 g, 10.496 mmol, 1.00 equiv) in toluene (50 mL) was added sodium methoxide (1.70 g, 31.488 mmol, 3.0 equiv) in portions at room temperature. The resulting mixture was stirred for 1 h at 80° C., acidified to pH 3 with 2 N aq. HCl, and extracted with ethyl acetate (3×50 mL). The organic layers were combined, washed with brine (1×50 mL), dried over anhydrous Na₂SO₄, and filtered. After filtration, the filtrate was concentrated under reduced pressure to give a residue. The residue was purified by silica gel column chromatography, eluted with PE:EA (4:1) to afford 5-chloro-8-iodo-2-methoxyquinoline (3 g, 89%) as a solid. LCMS (ES, m/z): 320 [M+H]⁺.

### Synthesis of Intermediate C34

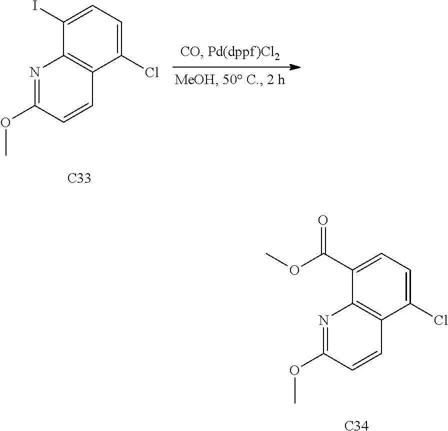 [SMILES: COc1ccc2c(Cl)ccc(I)c2n1 ; "C33" ; COC(=O)c1ccc(Cl)c2ccc(OC)nc12 ; "C34"]

To a solution of 5-chloro-8-iodo-2-methoxyquinoline (3 g, 9.389 mmol, 1 equiv) in methanol (50 mL) was added Pd(dppf)Cl₂·CH₂Cl₂ (0.76 g, 0.939 mmol, 0.1 equiv) and TEA (2.85 g, 28.167 mmol, 3.0 equiv) in a pressure tank. The reaction mixture was purged with nitrogen for 1 min, then was pressurized to 20 atm with carbon monoxide at 50° C. for 2 h. The resulting mixture was cooled to room temperature and filtered to remove insoluble solids, then concentrated under reduced pressure to give a residue. The residue was purified by silica gel column chromatography, eluted with PE:EA (3:7) to afford methyl 5-chloro-2-methoxyquinoline-8-carboxylate (2 g, 85%) as a solid. LCMS (ES, m/z): 252 [M+H]⁺.

### Synthesis of Intermediate C35

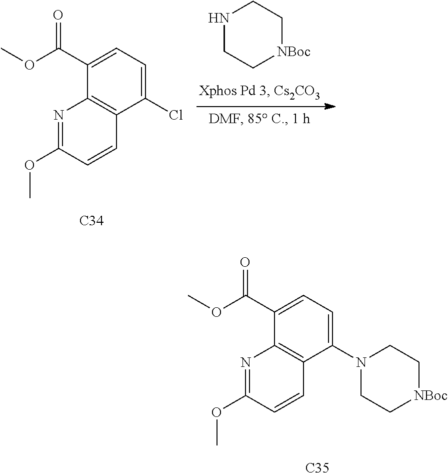 [SMILES: COC(=O)c1ccc(Cl)c2ccc(OC)nc12 ; CC(C)(C)OC(=O)N1CCNCC1 ; "C34" ; COC(=O)c1ccc(N2CCN(C(=O)OC(C)(C)C)CC2)c2ccc(OC)nc12 ; "C35"]

To a mixture of methyl 5-chloro-2-methoxyquinoline-8-carboxylate (1 g, 3.973 mmol, 1 equiv) and tert-butyl piperazine-1-carboxylate (1.48 g, 7.946 mmol, 2.0 equiv) in DMF (20 mL) was added Cs₂CO₃ (3.88 g, 11.919 mmol, 3.0 equiv) and XPhos Pd G3 (0.34 g, 0.397 mmol, 0.1 equiv). The reaction mixture was stirred for 1 h at 85° C. under a nitrogen atmosphere, then filtered and the filtrate purified by reverse flash chromatography (Condition 2, Gradient 1) to afford methyl 5-[4-(tert-butoxycarbonyl)piperazin-1-yl]-2-methoxyquinoline-8-carboxylate (1.5 g, 94%) as a solid. LCMS (ES, m/z): 402 [M+H]⁺.

### Synthesis of Intermediate C36

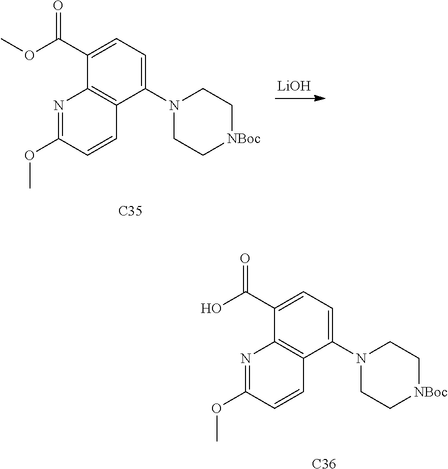

A mixture of methyl 5-[4-(tert-butoxycarbonyl)piperazin-1-yl]-2-methoxyquinoline-8-carboxylate (800 mg, 1.993 mmol, 1 equiv) and LiOH·H2O (334 mg, 7.972 mmol, 4.0 equiv) in methanol (5 mL), THF (5 mL), and water (5 mL) was stirred for 1 h at room temperature. The mixture was acidified to pH 2 with 1 N HCl. A precipitate formed and was collected by filtration and washed with water (2×10 mL) to afford 5-(4-(tert-butoxycarbonyl)piperazin-1-yl)-2-methoxyquinoline-8-carboxylic acid (600 mg). LCMS (ES, m/z): 388 [M+H]⁺.

### Synthesis of Intermediate C37

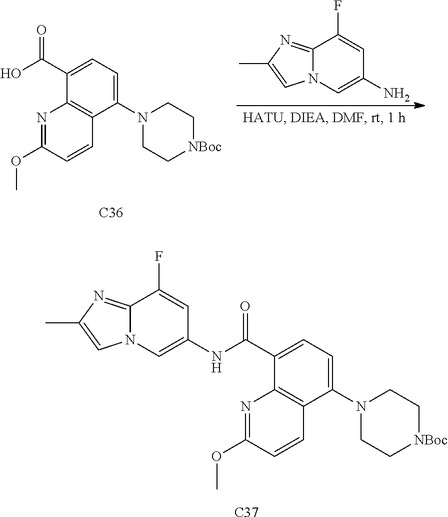 [SMILES: COc1ccc2c(N3CCN(C(=O)OC(C)(C)C)CC3)ccc(C(=O)O)c2n1 ; Cc1cn2cc(N)cc(F)c2n1 ; "C36" ; COc1ccc2c(N3CCN(C(=O)OC(C)(C)C)CC3)ccc(C(=O)Nc3cc(F)c4nc(C)cn4c3)c2n1 ; "C37"]

To a stirred solution of 5-[4-(tert-butoxycarbonyl)piperazin-1-yl]-2-methoxyquinoline-8-carboxylic acid (40 mg, 0.103 mmol, 1 equiv) and HATU (47 mg, 0.124 mmol, 1.2 equiv) in DMF (2.0 mL) was added DIEA (40 mg, 0.309 mmol, 3.0 equiv) and 8-fluoro-2-methylimidazo[1,2-a]pyridin-6-amine (18.76 mg, 0.113 mmol, 1.1 equiv) in portions at room temperature. The resulting mixture was stirred for 1 h at room temperature, then purified by reverse flash chromatography (Condition 2, Gradient 2) to afford tert-butyl 4-[8-({8-fluoro-2-methylimidazo[1,2-a]pyridin-6-yl}carbamoyl)-2-methoxyquinolin-5-yl]piperazine-1-carboxylate (30 mg, 54%) as a solid. LCMS (ES, m/z): 535 [M+H]⁺.

### Synthesis of Compound 169

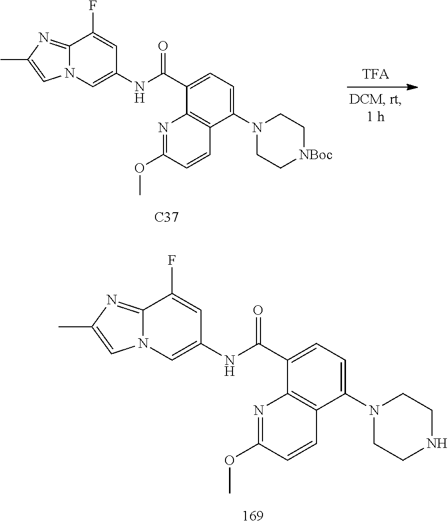 [SMILES: COc1ccc2c(N3CCN(C(=O)OC(C)(C)C)CC3)ccc(C(=O)Nc3cc(F)c4nc(C)cn4c3)c2n1 ; "C37" ; COc1ccc2c(N3CCNCC3)ccc(C(=O)Nc3cc(F)c4nc(C)cn4c3)c2n1]

A mixture of tert-butyl 4-[8-({8-fluoro-2-methylimidazo[1,2-a]pyridin-6-yl}carbamoyl)-2-methoxyquinolin-5-yl]piperazine-1-carboxylate (30 mg, 0.056 mmol, 1.00 equiv) and TFA (1 mL) in DCM (2 mL) was stirred for 1 h at room temperature, then concentrated under reduced pressure to give a residue. The residue was purified by reverse flash chromatography (Condition 1, Gradient 3) to afford N-{8-fluoro-2-methylimidazo[1,2-a]pyridin-6-yl}-2-methoxy-5-(piperazin-1-yl)quinoline-8-carboxamide (6.2 mg, 25%) as a solid. LCMS (ES, m/z): 435 [M+H]⁺. ¹H NMR (300 MHz, DMSO-d₆) δ 12.13 (s, 1H), 9.20 (d, J=1.6 Hz, 1H), 8.54-8.42 (m, 2H), 7.96-7.89 (m, 1H), 7.27 (dd, J=12.6, 1.6 Hz, 1H), 7.18 (t, J=8.5 Hz, 2H), 4.18 (s, 3H), 3.00 (d, J=9.3 Hz, 8H), 2.39-2.33 (m, 3H).

### Example 66: Synthesis of Compound 163

### Synthesis of Intermediate C38

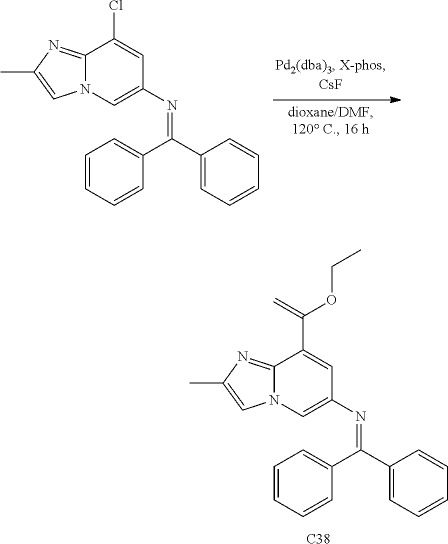 [SMILES: Cc1cn2cc(N=C(c3ccccc3)c3ccccc3)cc(Cl)c2n1 ; C=C(OCC)c1cc(N=C(c2ccccc2)c2ccccc2)cn2cc(C)nc12 ; "C38"]

To a stirred mixture of N-{8-chloro-2-methylimidazo[1,2-a]pyridin-6-yl}-1,1-diphenylmethanimine (500.0 mg, 1.446 mmol, 1.0 equiv) and tributyl(1-ethoxyethenyl)stannane (783.2 mg, 2.169 mmol, 1.5 equiv) in dioxane (10 mL) and DMF (2.5 mL) was added CsF (658.8 mg, 4.338 mmol, 3.0 equiv), X-phos (137.8 mg, 0.289 mmol, 0.2 equiv), and Pd₂(dba)₃ (132.4 mg, 0.145 mmol, 0.1 equiv) at room temperature under nitrogen atmosphere. The resulting mixture was stirred for 16 h at 120° C. under nitrogen atmosphere. The resulting mixture was diluted with water (50 mL) and extracted with ethyl acetate (2×50 mL). The organic layers were combined, washed with water (1×100 mL), dried over anhydrous Na₂SO₄, and filtered. After filtration, the filtrate was concentrated under reduced pressure to give a residue. The residue was purified by silica gel column chromatography, eluted with PE/EA (3:1) to afford N-[8-(1-ethoxyethenyl)-2-methylimidazo[1,2-a]pyridin-6-yl]-1,1-diphenylmethanimine (400 mg, 73%) as an oil. LCMS (ES, m/z):382 [M+H]⁺.

### Synthesis of Intermediate C39

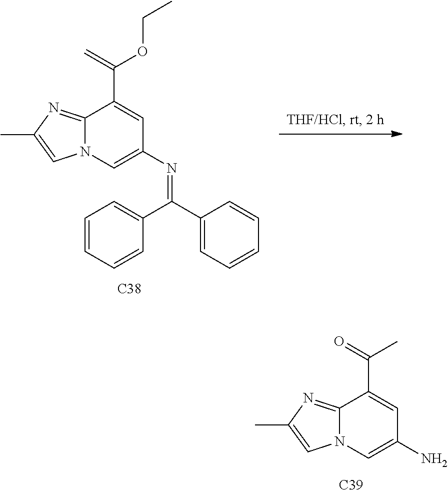 [SMILES: C=C(OCC)c1cc(N=C(c2ccccc2)c2ccccc2)cn2cc(C)nc12 ; "THF/HCl, rt, 2 h" ; "C38" ; CC(=O)c1cc(N)cn2cc(C)nc12 ; "C39"]

To a stirred mixture of N-[8-(1-ethoxyethenyl)-2-methylimidazo[1,2-a]pyridin-6-yl]-1,1-diphenylmethanimine (400.0 mg, 1.049 mmol, 1.0 equiv) in THE (2 mL) was added HCl (2 mL, conc.) dropwise at room temperature. The resulting mixture was stirred for 2 h at room temperature, then concentrated under vacuum to give a residue. The residue was neutralized to pH 8 with saturated NaHCO₃ (aq.) and extracted with ethyl acetate (2×3 mL). The organic layers were combined, washed with water (1×5 mL), dried over anhydrous Na₂SO₄, and filtered.

After filtration, the filtrate was concentrated under reduced pressure to give a residue. The residue was purified by silica gel column chromatography, eluted with PE/EA (1:2) to afford 1-{6-amino-2-methylimidazo[1,2-a]pyridin-8-yl}ethanone (100 mg, 50%) as a solid. LCMS (ES, m/z):190 [M+H]⁺.

### Synthesis of Intermediate C40

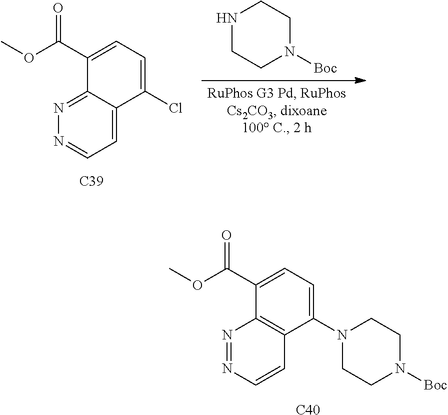

To a stirred mixture of methyl 5-chlorocinnoline-8-carboxylate (90.0 mg, 0.404 mmol, 1.0 equiv) and tert-butyl piperazine-1-carboxylate (112.9 mg, 0.606 mmol, 1.5 equiv) in dioxane (1 mL) was added Cs₂CO₃ (395.1 mg, 1.212 mmol, 3.0 equiv), RuPhos (37.7 mg, 0.081 mmol, 0.2 equiv), and RuPhos Palladacycle Gen.3 (33.8 mg, 0.040 mmol, 0.1 equiv) at room temperature under nitrogen atmosphere. The resulting mixture was stirred for 2 h at 100° C. under nitrogen atmosphere, then concentrated under vacuum to give a residue. The residue was purified by silica gel column chromatography, eluted with PE/EA (1:1) to afford methyl 5-[4-(tert-butoxycarbonyl)piperazin-1-yl]cinnoline-8-carboxylate (110 mg, 73%) as an oil. LCMS (ES, m/z):373 [M+H]⁺.

### Synthesis of Intermediate C41

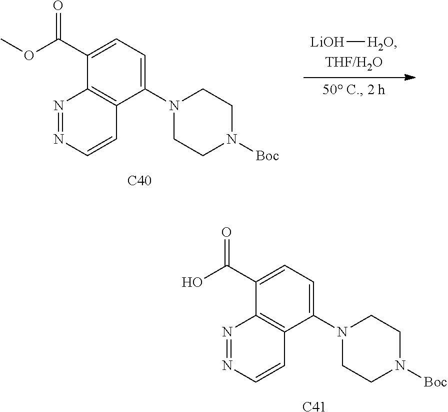

To a stirred mixture of methyl 5-[4-(tert-butoxycarbonyl)piperazin-1-yl]cinnoline-8-carboxylate (110.0 mg, 0.295 mmol, 1.0 equiv) in THE (2 mL) and water (1 mL) was added lithiumol hydrate (37.2 mg, 0.885 mmol, 3.0 equiv) at room temperature. The resulting mixture was stirred for 2 h at 50° C., then neutralized to pH 7 with 1 N HCl (aq.) and extracted with ethyl acetate (3×5 mL). The organic layers were combined, dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford 5-[4-(tert-butoxycarbonyl)piperazin-1-yl]cinnoline-8-carboxylic acid (100 mg, 94%) as a solid. LCMS (ES, m/z):357 [M−H]⁺.

### Synthesis of Intermediate C42

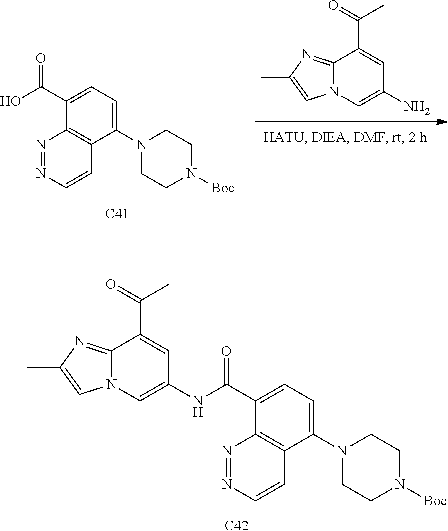 [SMILES: CC(C)(C)OC(=O)N1CCN(c2ccc(C(=O)O)c3nnccc23)CC1 ; CC(=O)c1cc(N)cn2cc(C)nc12 ; "C41" ; CC(=O)c1cc(NC(=O)c2ccc(N3CCN(C(=O)OC(C)(C)C)CC3)c3ccnnc23)cn2cc(C)nc12 ; "C42"]

To a stirred mixture of 5-[4-(tert-butoxycarbonyl)piperazin-1-yl]cinnoline-8-carboxylic acid (50.0 mg, 0.140 mmol, 1.0 equiv) and 1-{6-amino-2-methylimidazo[1,2-a]pyridin-8-yl}ethanone (26.4 mg, 0.140 mmol, 1.0 equiv) in DMF (1 mL) was added DIEA (54.1 mg, 0.420 mmol, 3.0 equiv) and HATU (63.6 mg, 0.168 mmol, 1.2 equiv) at room temperature. The resulting mixture was stirred for 2 h at room temperature, then diluted with water (3 mL) and extracted with ethyl acetate (3×2 mL). The organic layers were combined, washed with water (1×4 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10/1) to afford tert-butyl 4-[8-({8-acetyl-2-methylimidazo[1,2-a]pyridin-6-yl}carbamoyl)cinnolin-5-yl]piperazine-1-carboxylate (45 mg, 61%) as a solid. LCMS (ES, m/z): 530 [M+H]⁺.

### Synthesis of Compound 163

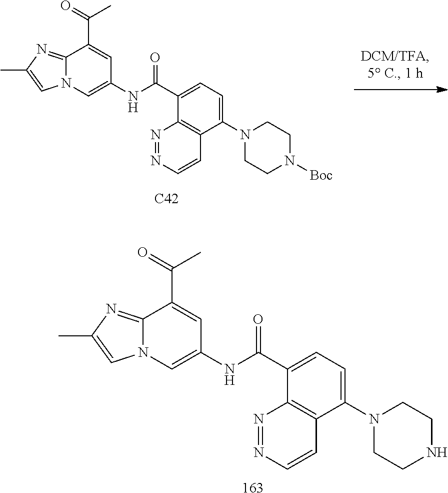

To a stirred mixture of tert-butyl 4-[8-({8-acetyl-2-methylimidazo[1,2-a]pyridin-6-yl}carbamoyl)cinnolin-5-yl]piperazine-1-carboxylate (45.0 mg, 0.085 mmol, 1.0 equiv) in DCM (1 mL) was added TFA (0.3 mL) dropwise at 5° C. The resulting mixture was stirred for 1 h at 5° C., then concentrated under vacuum to give a residue. The residue was purified by Prep-HPLC (Condition 2, Gradient 5) to afford N-{8-acetyl-2-methylimidazo[1,2-a]pyridin-6-yl}-5-(piperazin-1-yl)cinnoline-8-carboxamide (30 mg, 65%) as a solid. LCMS (ES, m/z):430 [M+H]⁺. ¹H NMR (400 MHz, DMSO-d₆) δ 12.35 (s, 1H), 9.95 (s, 1H), 9.58 (d, J=5.9 Hz, 1H), 9.01-9.00 (m, 2H), 8.62-8.61 (m, 1H), 8.53-8.51 (m, 2H), 8.30 (s, 1H), 7.61 (d, J=8.0 Hz, 1H), 3.41-3.39 (m, 8H), 2.84 (s, 3H), 2.51 (s, 3H).

### Example 67: Synthesis of Compound 168

### Synthesis of Intermediate C43

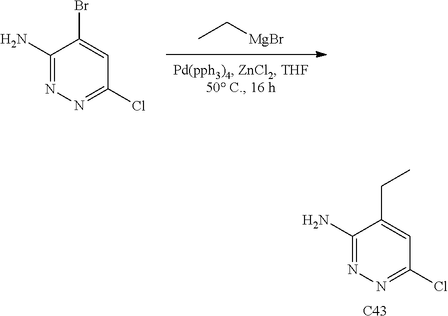

To a stirred mixture of 4-bromo-6-chloropyridazin-3-amine (3.0 g, 14.393 mmol, 1.0 equiv) in THE (30 mL) was added ethylmagnesium bromide (7.2 mL, 2 mol/L) dropwise at 0° C. under nitrogen atmosphere. To the reaction mixture was added Pd(PPh₃)₄(0.17 g, 0.144 mmol, 0.01 equiv) and zinc chloride (2.75 g, 20.150 mmol, 1.4 equiv) portionwise over 5 min at room temperature. The resulting mixture was stirred for 30 min at room temperature. To the resulting mixture was added ethylmagnesium bromide (13.1 mL, 2 mol/L) dropwise over 5 min at 50° C. The resulting mixture was stirred for 16 h at 50° C., then quenched with ice water (50 mL) and extracted with ethyl acetate (3×50 mL). The organic layers were combined, washed with brine (3×30 mL), dried over anhydrous Na₂SO₄, and filtered. After filtration, the filtrate was concentrated under reduced pressure to give a residue. The residue was purified by silica gel column chromatography, eluted with PE/EA (1:1) to afford 6-chloro-4-ethylpyridazin-3-amine (890 mg, 39%) as a solid. LCMS (ES, m/z):158 [M+H]⁺.

### Synthesis of Intermediate C44

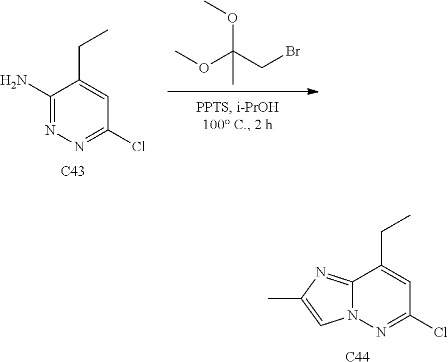

To a stirred mixture of 6-chloro-4-ethylpyridazin-3-amine (0.90 g, 5.711 mmol, 1.0 equiv) and 1-bromo-2,2-dimethoxypropane (1.25 g, 6.853 mmol, 1.2 equiv) in isopropanol (18 mL) was added PPTS (100.5 mg, 0.400 mmol, 0.07 equiv) at room temperature. The resulting mixture was stirred for 2 h at 100° C., then diluted with water (20 mL) and extracted with ethyl acetate (3×20 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:1) to afford 6-chloro-8-ethyl-2-methylimidazo[1,2-b]pyridazine (490 mg, 44%) as a solid. LCMS (ES, m/z):196 [M+H]⁺.

### Synthesis of Intermediate C45

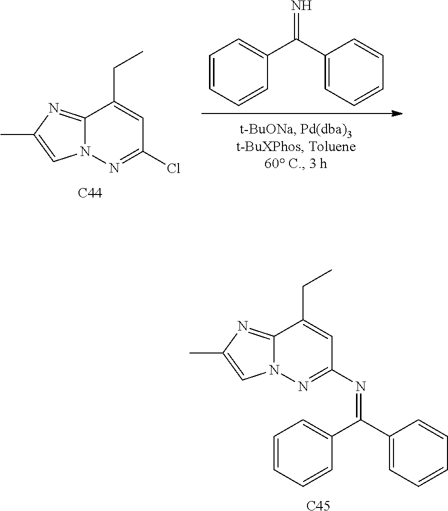 [SMILES: CCc1cc(Cl)nn2cc(C)nc12 ; N=C(c1ccccc1)c1ccccc1 ; "C44" ; CCc1cc(N=C(c2ccccc2)c2ccccc2)nn2cc(C)nc12 ; "C45"]

To a stirred mixture of 6-chloro-8-ethyl-2-methylimidazo[1,2-b]pyridazine (490 mg, 2.504 mmol, 1.0 equiv) and α-phenyl-benzenemethanimine (453.9 mg, 2.504 mmol, 1.0 equiv) in toluene (7.5 mL) was added t-BuONa (722.1 mg, 7.512 mmol, 3.0 equiv), Pd₂(dba)₃ (229.3 mg, 0.250 mmol, 0.1 equiv), and t-BuXPhos (212.7 mg, 0.501 mmol, 0.2 equiv) at room temperature under nitrogen atmosphere. The resulting mixture was stirred for 3 h at 60° C. under nitrogen atmosphere, then concentrated under reduced pressure to give a residue. The residue was purified by silica gel column chromatography, eluted with PE/EA (2:1) to afford N-{8-ethyl-2-methylimidazo[1,2-b]pyridazin-6-yl}-1,1-diphenylmethanimine (450 mg, 53%) as a solid. LCMS (ES, m/z):341 [M+H]⁺.

### Synthesis of Intermediate C46

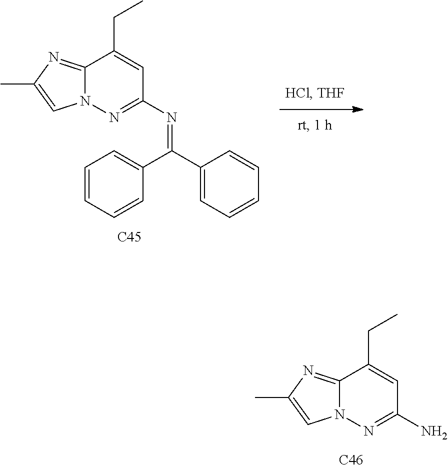 [SMILES: CCc1cc(N=C(c2ccccc2)c2ccccc2)nn2cc(C)nc12 ; "C45" ; CCc1cc(N)nn2cc(C)nc12 ; "C46"]

To a stirred solution of N-{8-ethyl-2-methylimidazo[1,2-b]pyridazin-6-yl}-1,1-diphenylmethanimine (450 mg, 1.322 mmol, 1.0 equiv) in THE (4.5 mL) was added HCl (4.5 mL, con.) dropwise at room temperature. The resulting mixture was stirred for 1 h at room temperature, then concentrated under vacuum, basified to pH 9 with saturated Na₂CO₃ (aq.), and extracted with ethyl acetate (3×20 mL). The organic layers were combined, dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford 8-ethyl-2-methylimidazo[1,2-b]pyridazin-6-amine (150 mg, 64%) as a solid. LCMS (ES, m/z):177 [M+H]⁺.

### Synthesis of Intermediate C47

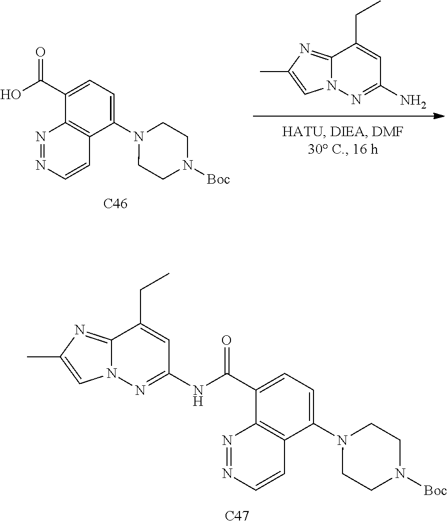 [SMILES: CC(C)(C)OC(=O)N1CCN(c2ccc(C(=O)O)c3nnccc23)CC1 ; CCc1cc(N)nn2cc(C)nc12 ; "C46" ; CCc1cc(NC(=O)c2ccc(N3CCN(C(=O)OC(C)(C)C)CC3)c3ccnnc23)nn2cc(C)nc12 ; "C47"]

To a stirred solution of 5-[4-(tert-butoxycarbonyl)piperazin-1-yl]cinnoline-8-carboxylic acid (50 mg, 0.140 mmol, 1.0 equiv) and HATU (79.6 mg, 0.210 mmol, 1.5 equiv) in DMF (1 mL) was added DIEA (54.1 mg, 0.420 mmol, 3.0 equiv) and 8-ethyl-2-methylimidazo[1,2-b]pyridazin-6-amine (24.6 mg, 0.140 mmol, 1.0 equiv) in portions at room temperature. The resulting mixture was stirred for 16 h at 30° C. The resulting mixture was diluted with water (5 mL) and extracted with ethyl acetate (3×5 mL). The organic layers were combined, washed with water (3×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[8-({8-ethyl-2-methylimidazo[1,2-b]pyridazin-6-yl}carbamoyl)cinnolin-5-yl]piperazine-1-carboxylate (50 mg, 69%) as a solid. LCMS (ES, m/z):517 [M+H]⁺.

### Synthesis of Compound 168

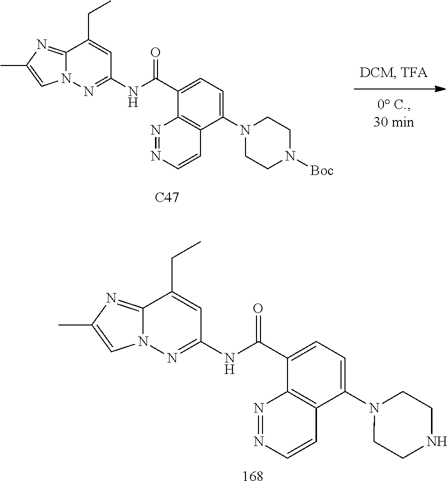

To a stirred solution of tert-butyl 4-[8-({8-ethyl-2-methylimidazo[1,2-b]pyridazin-6-yl}carbamoyl)cinnolin-5-yl]piperazine-1-carboxylate (50 mg, 0.097 mmol, 1.0 equiv) in DCM (2 mL) was added TFA (0.6 mL) dropwise at 0° C. The resulting mixture was stirred for 30 min at 0° C., then concentrated under reduced pressure to give a residue. The residue was purified by Prep-HPLC (Condition 2, Gradient 3) to afford N-{8-ethyl-2-methylimidazo[1,2-b]pyridazin-6-yl}-5-(piperazin-1-yl)cinnoline-8-carboxamide trifluoroacetic acid salt (10.5 mg, 26%) as a solid. LCMS (ES, m/z):417 [M+H]⁺. ¹H NMR (300 MHz, DMSO-d₆) δ 13.48 (s, 1H), 9.60 (d, J=5.9 Hz, 1H), 8.87-8.86 (m, 2H), 8.76 (d, J=8.1 Hz, 1H), 8.55 (d, J=6.1 Hz, 1H), 8.28 (s, 1H), 8.11 (s, 1H), 7.63 (d, J=8.1 Hz, 1H), 3.44-3.42 (m, 8H), 3.04 (q, J=7.6 Hz, 2H), 2.44 (s, 3H), 1.39 (t, J=7.5 Hz, 3H).

### Example 68: Synthesis of Compound 164

### Synthesis of Intermediate C48

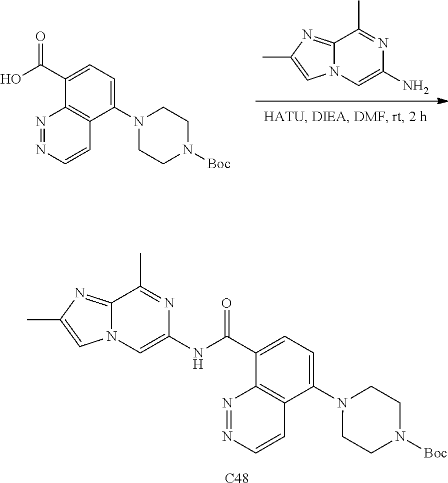 [SMILES: CC(C)(C)OC(=O)N1CCN(c2ccc(C(=O)O)c3nnccc23)CC1 ; Cc1cn2cc(N)nc(C)c2n1 ; Cc1cn2cc(NC(=O)c3ccc(N4CCN(C(=O)OC(C)(C)C)CC4)c4ccnnc34)nc(C)c2n1 ; "C48"]

To a stirred mixture of 5-[4-(tert-butoxycarbonyl)piperazin-1-yl]cinnoline-8-carboxylic acid (50.0 mg, 0.140 mmol, 1.0 equiv) and 2,8-dimethylimidazo[1,2-a]pyrazin-6-amine (22.6 mg, 0.140 mmol, 1.0 equiv) in DMF (1 mL) was added DIEA (54.1 mg, 0.420 mmol, 3.0 equiv) and HATU (63.6 mg, 0.168 mmol, 1.2 equiv) at room temperature. The resulting mixture was stirred for 2 h at room temperature, then diluted with water (3 mL) and extracted with ethyl acetate (3×2 mL). The organic layers were combined, washed with water (1×4 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10/1) to afford tert-butyl 4-[8-({2,8-dimethylimidazo[1,2-a]pyrazin-6-yl}carbamoyl)cinnolin-5-yl]piperazine-1-carboxylate (23 mg, 33%) as a solid. LCMS (ES, m/z):503 [M+H]⁺.

### Synthesis of Compound 164

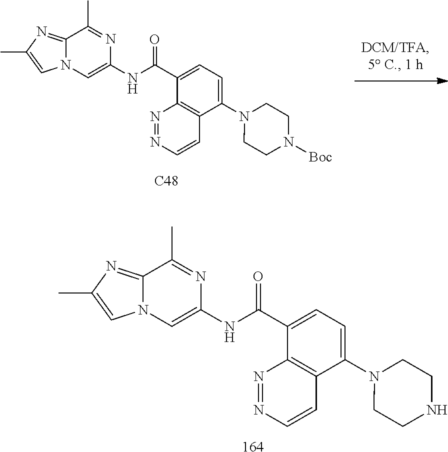

To a stirred solution of tert-butyl 4-[8-({2,8-dimethylimidazo[1,2-a]pyrazin-6-yl}carbamoyl)cinnolin-5-yl]piperazine-1-carboxylate (23.0 mg, 0.046 mmol, 1.0 equiv) in DCM (1 mL) was added TFA (0.3 mL) dropwise at 5° C. The resulting mixture was stirred for 1 h at 5° C., then concentrated under vacuum to give a residue. The residue was purified by Prep-HPLC (Condition 3, Gradient 14) to afford N-{2,8-dimethylimidazo[1,2-a]pyrazin-6-yl}-5-(piperazin-1-yl)cinnoline-8-carboxamide 2,2,2-trifluoroacetate (16 mg, 71%) as a solid. LCMS (ES, m/z):421 [M+H]⁺. ¹H NMR (400 MHz, DMSO-d₆) δ 13.20 (s, 1H), 9.59 (d, J=5.9 Hz, 1H), 9.44 (s, 1H), 8.93 (s, 2H), 8.77 (d, J=8.1 Hz, 1H), 8.55 (d, J=6.0 Hz, 1H), 8.11 (s, 1H), 7.63 (d, J=8.1 Hz, 1H), 3.46 (s, 4H), 3.41 (d, J=5.3 Hz, 4H), 2.77 (s, 3H), 2.45 (s, 3H).

### Example 70: Synthesis of Compound 171

### Synthesis of Intermediate C53

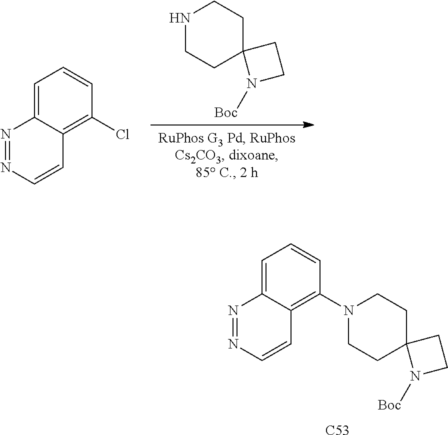

To a stirred mixture of 5-chlorocinnoline (140 mg, 0.851 mmol, 1.0 equiv) and tert-butyl 1,7-diazaspiro[3.5]nonane-1-carboxylate (288.7 mg, 1.276 mmol, 1.5 equiv) in dioxane (3.5 mL) was added Cs₂CO₃ (831.4 mg, 2.553 mmol, 3.0 equiv), RuPhos (79.4 mg, 0.170 mmol, 0.2 equiv), and RuPhos Palladacycle Gen.3 (71.1 mg, 0.085 mmol, 0.1 equiv) at room temperature under nitrogen atmosphere. The resulting mixture was stirred for 2 h at 85° C. under nitrogen atmosphere, then concentrated under vacuum to give a residue. The residue was purified by silica gel column chromatography, eluted with CH₂Cl₂/MeOH (10:1) to afford tert-butyl 7-(cinnolin-5-yl)-1,7-diazaspiro[3.5]nonane-1-carboxylate (350 mg, 99%) as a solid. LCMS (ES, m/z):355 [M+H]⁺.

### Synthesis of Intermediate C54

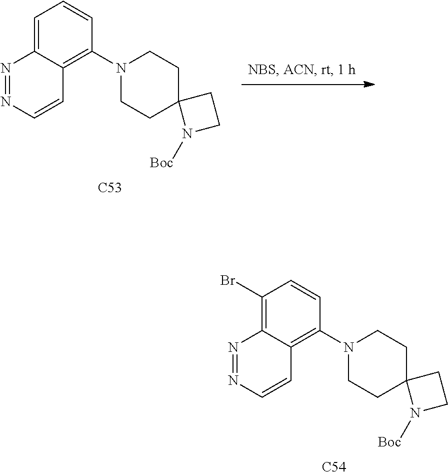 [SMILES: CC(C)(C)OC(=O)N1CCC12CCN(c1cccc3nnccc13)CC2 ; "NBS, ACN, rt, 1 h" ; "C53" ; CC(C)(C)OC(=O)N1CCC12CCN(c1ccc(Br)c3nnccc13)CC2 ; "C54"]

To a stirred solution of tert-butyl 7-(cinnolin-5-yl)-1,7-diazaspiro[3.5]nonane-1-carboxylate (340 mg, 0.959 mmol, 1.0 equiv) in acetonitrile (6.8 mL) was added NBS (170.7 mg, 0.959 mmol, 1.0 equiv) at room temperature. The resulting mixture was stirred for 1 h at room temperature, then concentrated under reduced pressure. The resulting mixture was diluted with water (10 mL) and extracted with ethyl acetate (3×10 mL). The organic layers were combined, dried over anhydrous Na₂SO₄, and filtered. After filtration, the filtrate was concentrated under reduced pressure to afford tert-butyl 7-(8-bromocinnolin-5-yl)-1,7-diazaspiro[3.5]nonane-1-carboxylate (320 mg, 77%) as a solid. LCMS (ES, m/z):433 [M+H]⁺.

### Synthesis of Intermediate C55

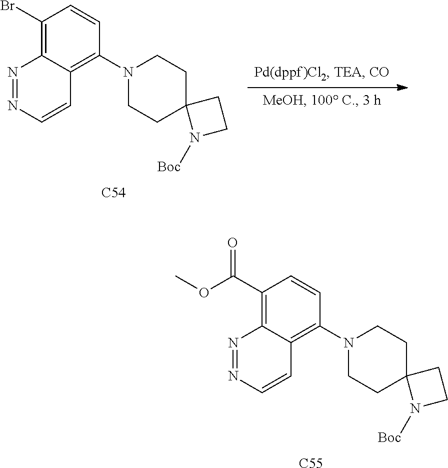 [SMILES: CC(C)(C)OC(=O)N1CCC12CCN(c1ccc(Br)c3nnccc13)CC2 ; "MeOH, 100° C., 3 h" ; "Pd(dppf)Cl2, TEA, CO" ; "C54" ; COC(=O)c1ccc(N2CCC3(CC2)CCN3C(=O)OC(C)(C)C)c2ccnnc12 ; "C55"]

To a solution of tert-butyl 7-(8-bromocinnolin-5-yl)-1,7-diazaspiro[3.5]nonane-1-carboxylate (290 mg, 0.669 mmol, 1.0 equiv) in methanol (15 mL) was added TEA (203.2 mg, 2.007 mmol, 3.0 equiv) and Pd(dppf)Cl₂·CH₂Cl₂ (49.0 mg, 0.067 mmol, 0.1 equiv) in a pressure tank. The reaction mixture was purged with nitrogen for 5 min, then was pressurized to 1 MPa with carbon monoxide at 100° C. for 3 h. The reaction mixture was cooled to room temperature and filtered to remove insoluble solids. The resulting mixture was concentrated under vacuum to give a residue. The residue was purified by silica gel column chromatography, eluted with PE/EA (1:1) to afford methyl 5-[1-(tert-butoxycarbonyl)-1,7-diazaspiro[3.5]nonan-7-yl]cinnoline-8-carboxylate (220 mg, 80%) as a solid. LCMS (ES, m/z): 413 [M+H]⁺.

### Synthesis of Intermediate C56

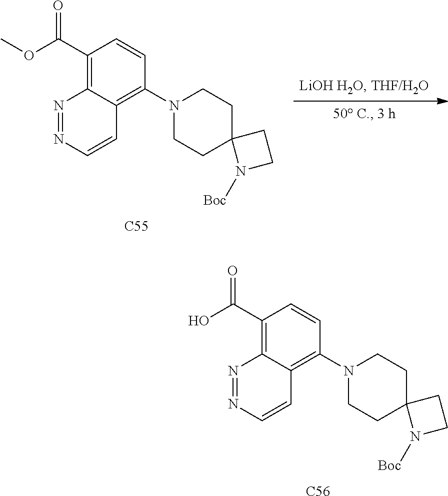 [SMILES: COC(=O)c1ccc(N2CCC3(CC2)CCN3C(=O)OC(C)(C)C)c2ccnnc12 ; "50° C., 3 h" ; "LiOH H2O, THF/H2O" ; "C55" ; CC(C)(C)OC(=O)N1CCC12CCN(c1ccc(C(=O)O)c3nnccc13)CC2 ; "C56"]

To a stirred mixture of methyl 5-[1-(tert-butoxycarbonyl)-1,7-diazaspiro[3.5]nonan-7-yl]cinnoline-8-carboxylate (220 mg, 0.533 mmol, 1.0 equiv) in THE (2.5 mL) and water (2.5 mL) was added LiOH·H₂O (111.9 mg, 2.665 mmol, 5.0 equiv) at room temperature. The resulting mixture was stirred for 3 h at 50° C., then diluted with water (5 mL), acidified to pH 6 with HCl (1 N), and extracted with ethyl acetate (3×10 mL). The organic layers were combined, dried over anhydrous Na₂SO₄, and filtered. After filtration, the filtrate was concentrated under reduced pressure to afford 5-[1-(tert-butoxycarbonyl)-1,7-diazaspiro[3.5]nonan-7-yl]cinnoline-8-carboxylic acid (240 mg, 99%) as a solid. LCMS (ES, m/z):399 [M+H]⁺.

### Synthesis of Intermediate C57

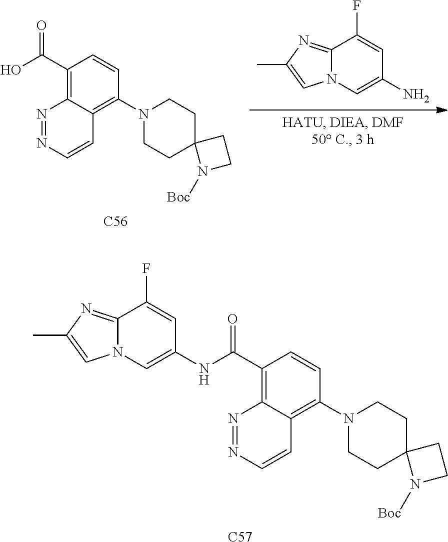

To a stirred solution of 5-[1-(tert-butoxycarbonyl)-1,7-diazaspiro[3.5]nonan-7-yl]cinnoline-8-carboxylic acid (240 mg, 0.602 mmol, 1.0 equiv) in DMF (5 mL) was added DIEA (233.5 mg, 1.806 mmol, 3.0 equiv), HATU (343.5 mg, 0.903 mmol, 1.5 equiv), and 8-fluoro-2-methylimidazo[1,2-a]pyridin-6-amine (109.4 mg, 0.662 mmol, 1.1 equiv) at room temperature. The resulting mixture was stirred for 3 h at 50° C., then diluted with water (15 mL) and extracted with ethyl acetate (3×15 mL). The organic layers were combined, washed with water (3×10 mL), dried over anhydrous Na₂SO₄, and filtered. After filtration, the filtrate was concentrated under reduced pressure to give a residue. The residue was purified by silica gel column chromatography, eluted with EA (100%) to afford tert-butyl 7-[8-({8-fluoro-2-methylimidazo[1,2-a]pyridin-6-yl}carbamoyl)cinnolin-5-yl]-1,7-diazaspiro[3.5]nonane-1-carboxylate (100 mg, 30%) as a solid. LCMS (ES, m/z):546 [M+H]⁺.

### Synthesis of Compound 171

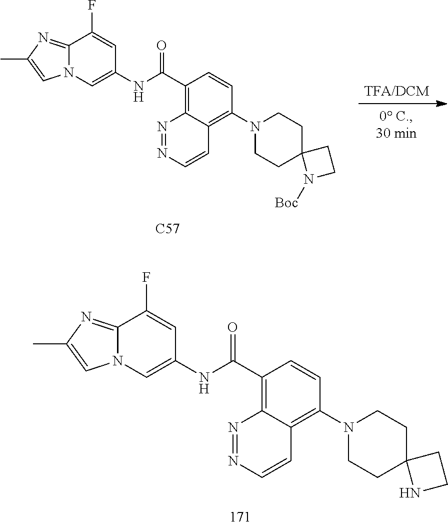 [SMILES: Cc1cn2cc(NC(=O)c3ccc(N4CCC5(CC4)CCN5C(=O)OC(C)(C)C)c4ccnnc34)cc(F)c2n1 ; "0° C.,30 min" ; "TFA/DCM" ; "C57" ; Cc1cn2cc(NC(=O)c3ccc(N4CCC5(CCN5)CC4)c4ccnnc34)cc(F)c2n1 ; "171"]

To a stirred solution of tert-butyl 7-[8-({8-fluoro-2-methylimidazo[1,2-a]pyridin-6-yl}carbamoyl)cinnolin-5-yl]-1,7-diazaspiro[3.5]nonane-1-carboxylate (90 mg, 0.165 mmol, 1.0 equiv) in DCM (1.8 mL) was added TFA (0.6 mL) dropwise at 0° C. The resulting mixture was stirred for 30 min at 0° C., then concentrated under reduced pressure to give a residue. The residue was purified by Prep-HPLC (Condition 2, Gradient 3) to afford 5-{1,7-diazaspiro[3.5]nonan-7-yl}-N-{8-fluoro-2-methylimidazo[1,2-a]pyridin-6-yl}cinnoline-8-carboxamide trifluoroacetate (11.9 mg, 16%) as a solid. LCMS (ES, m/z):446 [M+H]⁺. ¹H NMR (300 MHz, DMSO-d₆) δ 12.23 (s, 1H), 9.54 (d, J=5.9 Hz, 1H), 9.39 (d, J=1.6 Hz, 1H), 8.91-8.90 (m, 2H), 8.50 (d, J=8.0 Hz, 1H), 8.37 (d, J=6.0 Hz, 1H), 8.10 (d, J=2.9 Hz, 1H), 7.59 (d, J=12.1 Hz, 1H), 7.49 (d, J=8.1 Hz, 1H), 3.96-3.94 (s, 2H), 3.33-3.22 (m, 2H), 3.15 (d, J=7.7 Hz, 2H), 2.43-2.38 (m, 6H), 2.30-2.26 (m, 4H).

### Example 71: Synthesis of Compound 161

### Synthesis of Intermediate C58

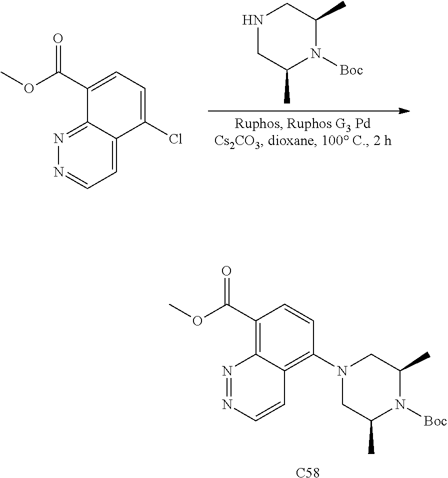

To a stirred mixture of methyl 5-chlorocinnoline-8-carboxylate (80.0 mg, 0.359 mmol, 1.0 equiv) and tert-butyl (2R,6S)-2,6-dimethylpiperazine-1-carboxylate (14.4 mg, 0.068 mmol, 1.5 equiv) in dioxane (2 mL) was added Cs₂CO₃ (351.2 mg, 1.077 mmol, 3.0 equiv), Ruphos (33.5 mg, 0.072 mmol, 0.2 equiv), and RuPhos Palladacycle Gen.3 (30.1 mg, 0.036 mmol, 0.1 equiv) at room temperature under nitrogen atmosphere. The resulting mixture was stirred for 2 h at 100° C. under nitrogen atmosphere. The resulting mixture was concentrated under vacuum to give a residue. The residue was purified by silica gel column chromatography, eluted with PE/EA (3:1) to afford methyl 5-[(3R,5S)-4-(tert-butoxycarbonyl)-3,5-dimethylpiperazin-1-yl]cinnoline-8-carboxylate (90 mg, 63%) as an oil. LCMS (ES, m/z):401 [M+H]⁺.

### Synthesis of Intermediate C59

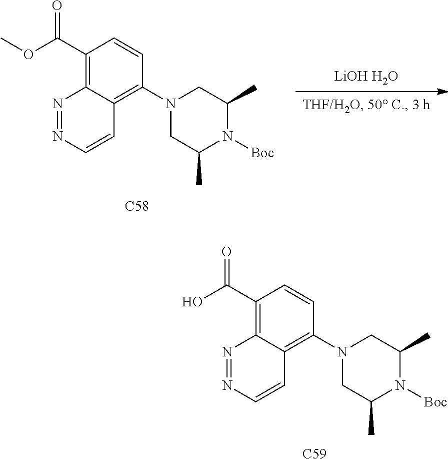 [SMILES: COC(=O)c1ccc(N2C[C@@H](C)N(C(=O)OC(C)(C)C)[C@@H](C)C2)c2ccnnc12 ; "LiOH H2O" ; "THF/H2O, 50° C., 3 h" ; "C58" ; C[C@@H]1CN(c2ccc(C(=O)O)c3nnccc23)C[C@H](C)N1C(=O)OC(C)(C)C ; "C59"]

To a stirred mixture of methyl 5-[(3R,5S)-4-(tert-butoxycarbonyl)-3,5-dimethylpiperazin-1-yl]cinnoline-8-carboxylate (90.0 mg, 0.225 mmol, 1.0 equiv) in THE (2 mL) and water (1 mL) was added lithiumol hydrate (28.3 mg, 0.675 mmol, 3.0 equiv) at room temperature. The resulting mixture was stirred for 3 h at 50° C., then neutralized to pH 7 with 1 N of HCl (aq.) and extracted with ethyl acetate (3×5 mL). The organic layers were combined, dried over anhydrous Na₂SO₄, and filtered. After filtration, the filtrate was concentrated under reduced pressure to afford 5-[(3R,5S)-4-(tert-butoxycarbonyl)-3,5-dimethylpiperazin-1-yl]cinnoline-8-carboxylic acid (80 mg, 92%) as a solid. LCMS (ES, m/z):387 [M+H]⁺.

### Synthesis of Intermediate C60

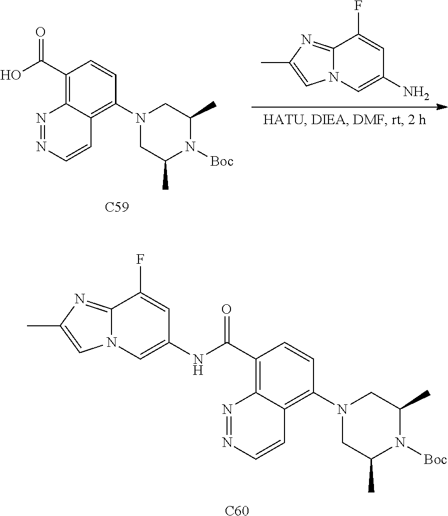

To a stirred mixture of 5-[(3R,5S)-4-(tert-butoxycarbonyl)-3,5-dimethylpiperazin-1-yl]cinnoline-8-carboxylic acid (40.0 mg, 0.104 mmol, 1.0 equiv) and 8-fluoro-2-methylimidazo[1,2-a]pyridin-6-amine (17.1 mg, 0.104 mmol, 1 equiv) in DMF (1 mL) was added DIEA (40.1 mg, 0.312 mmol, 3.0 equiv) and HATU (47.3 mg, 0.125 mmol, 1.2 equiv) at room temperature. The resulting mixture was stirred for 16 h at room temperature, then diluted with water (3 mL) and extracted with ethyl acetate (3×3 mL). The organic layers were combined, washed with water (1×5 mL), dried over anhydrous Na₂SO₄, and filtered. After filtration, the filtrate was concentrated under reduced pressure to give a residue. The residue was purified by silica gel column chromatography, eluted with EA to afford tert-butyl (2R,6S)-4-[8-({8-fluoro-2-methylimidazo[1,2-a]pyridin-6-yl}carbamoyl)cinnolin-5-yl]-2,6-dimethylpiperazine-1-carboxylate (30 mg, 54%) as a solid. LCMS (ES, m/z):534 [M+H]⁺.

### Synthesis of Compound 161

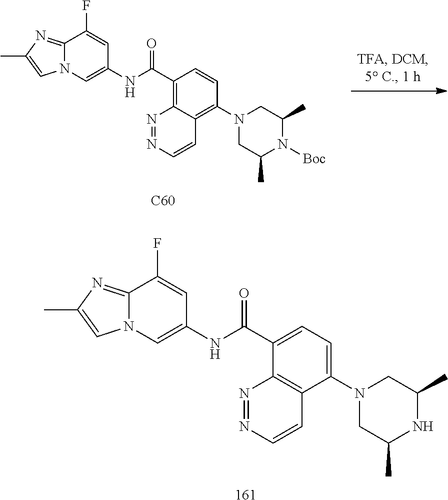

To a stirred solution of tert-butyl 4-[8-({8-fluoro-2-methylimidazo[1,2-a]pyridin-6-yl}carbamoyl)cinnolin-5-yl]-2,6-dimethylpiperazine-1-carboxylate (30.0 mg, 0.056 mmol, 1.0 equiv) in DCM (1 mL) was added TFA (0.3 mL) dropwise at 5° C. The resulting mixture was stirred for 2 h at room temperature, then concentrated under vacuum to give a residue. The residue was purified by Prep-HPLC (Condition 2, Gradient 7) to afford 5-(3,5-dimethylpiperazin-1-yl)-N-{8-fluoro-2-methylimidazo[1,2-a]pyridin-6-yl}cinnoline-8-carboxamide 2,2,2-trifluoroacetate (15 mg, 49%) as a solid. LCMS (ES, m/z):434 [M+H]⁺. ¹H NMR (400 MHz, DMSO-d₆) δ 12.17 (s, 1H), 9.56 (d, J=6.0 Hz, 1H), 9.38 (s, 1H), 9.29 (d, J=10.6 Hz, 1H), 8.58 (d, J=10.9 Hz, 1H), 8.51-8.49 (m, 2H), 8.10 (s, 1H), 7.63-7.57 (m, 2H), 3.77-3.74 (m, 2H), 3.65-3.61 (m, 2H), 2.92 (t, J=12.1 Hz, 2H), 2.42 (s, 3H), 1.31 (d, J=6.5 Hz, 6H).

### Example 72: Synthesis of Compound 176

### Synthesis of Intermediate C61

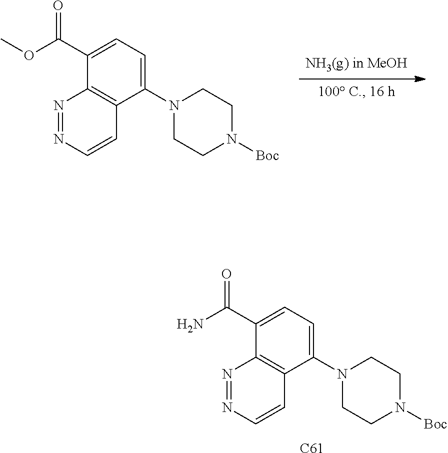 [SMILES: COC(=O)c1ccc(N2CCN(C(=O)OC(C)(C)C)CC2)c2ccnnc12 ; "100° C., 16 h" ; "NH3(g) in MeOH" ; CC(C)(C)OC(=O)N1CCN(c2ccc(C(N)=O)c3nnccc23)CC1 ; "C61"]

A mixture of methyl 5-[4-(tert-butoxycarbonyl)piperazin-1-yl]cinnoline-8-carboxylate (500 mg, 1.343 mmol, 1 equiv) in NH₃(g) in MeOH (20 mL) was stirred for 16 h at 100° C. The resulting mixture was concentrated under reduced pressure to give a residue. The residue was purified by silica gel column chromatography, eluted with PE/EA (1:1) to afford tert-butyl 4-(8-carbamoylcinnolin-5-yl)piperazine-1-carboxylate (350 mg, 73%) as a solid. LCMS (ES, m/z):358 [M+H]⁺.

### Synthesis of Intermediate C62

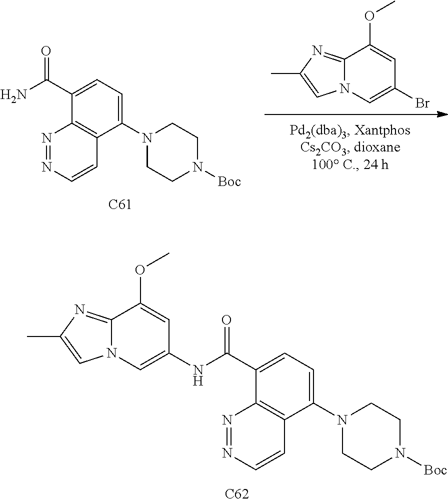 [SMILES: CC(C)(C)OC(=O)N1CCN(c2ccc(C(N)=O)c3nnccc23)CC1 ; COc1cc(Br)cn2cc(C)nc12 ; "C61" ; COc1cc(NC(=O)c2ccc(N3CCN(C(=O)OC(C)(C)C)CC3)c3ccnnc23)cn2cc(C)nc12 ; "C62"]

To a stirred mixture of tert-butyl 4-(8-carbamoylcinnolin-5-yl)piperazine-1-carboxylate (100 mg, 0.280 mmol, 1.0 equiv) and 6-bromo-8-methoxy-2-methylimidazo[1,2-a]pyridine (101.2 mg, 0.420 mmol, 1.5 equiv) in dioxane (2 mL) was added Cs₂CO₃ (273.48 mg, 0.840 mmol, 3.0 equiv), Xantphos (32.4 mg, 0.056 mmol, 0.2 equiv), and Pd₂(dba)₃ (25.6 mg, 0.028 mmol, 0.1 equiv) at room temperature under nitrogen atmosphere. The resulting mixture was stirred for 24 h at 100° C. under nitrogen atmosphere, then concentrated under vacuum to give a residue. The residue was purified by silica gel column chromatography, eluted with PE/EA (1:1) to afford tert-butyl 4-[8-({8-methoxy-2-methylimidazo[1,2-a]pyridin-6-yl}carbamoyl)cinnolin-5-yl]piperazine-1-carboxylate (62 mg, 43%) as a solid. LCMS (ES, m/z):518 [M+H]⁺.

### Synthesis of Compound 176

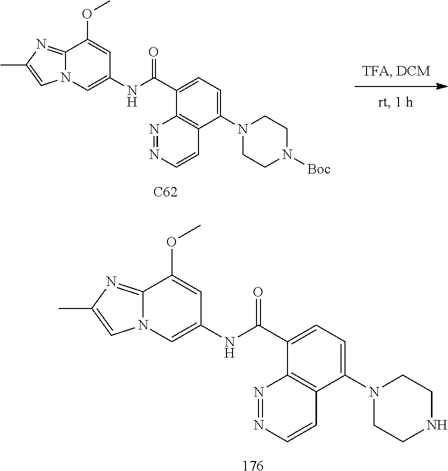 [SMILES: COc1cc(NC(=O)c2ccc(N3CCN(C(=O)OC(C)(C)C)CC3)c3ccnnc23)cn2cc(C)nc12 ; "C62" ; COc1cc(NC(=O)c2ccc(N3CCNCC3)c3ccnnc23)cn2cc(C)nc12 ; "176"]

To a stirred solution of tert-butyl 4-[8-({8-methoxy-2-methylimidazo[1,2-a]pyridin-6-yl}carbamoyl)cinnolin-5-yl]piperazine-1-carboxylate (60 mg, 0.116 mmol, 1.0 equiv) in DCM (1.8 mL) was added TFA (0.6 mL) dropwise at 0° C. The resulting mixture was stirred for 1 h at 0° C., then concentrated under reduced pressure to give a residue. The residue was purified by Prep-HPLC (Condition 2, Gradient 3) to afford N-{8-methoxy-2-methylimidazo[1,2-a]pyridin-6-yl}-5-(piperazin-1-yl)cinnoline-8-carboxamide trifluoroacetate (21.7 mg, 45%) as a solid. LCMS (ES, m/z):418.2 [M+H]⁺. ¹H NMR (400 MHz, DMSO-d₆) δ 9.53 (d, J=5.9 Hz, 1H), 9.39 (dt, J=3.0, 1.6 Hz, 1H), 8.85 (ddd, J=8.1, 3.4, 1.8 Hz, 1H), 8.58 (d, J=6.0 Hz, 1H), 7.97 (d, J=1.8 Hz, 1H), 7.65 (d, J=8.1 Hz, 1H), 7.61 (dt, J=3.3, 1.7 Hz, 1H), 4.22 (s, 3H), 3.61 (dd, J=6.6, 3.5 Hz, 4H), 3.51 (dd, J=6.6, 3.4 Hz, 4H), 2.54 (s, 3H).

### Example 73: Synthesis of Compound 177

### Synthesis of Intermediate C63

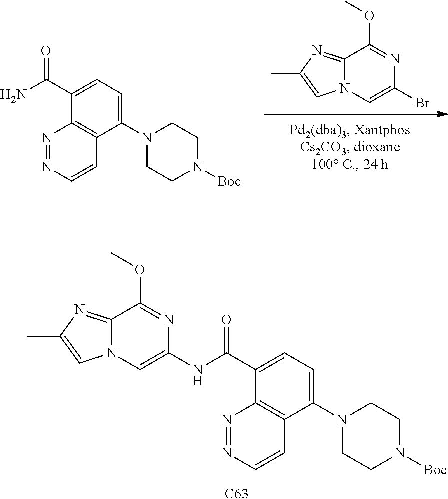 [SMILES: CC(C)(C)OC(=O)N1CCN(c2ccc(C(N)=O)c3nnccc23)CC1 ; COc1nc(Br)cn2cc(C)nc12 ; COc1nc(NC(=O)c2ccc(N3CCN(C(=O)OC(C)(C)C)CC3)c3ccnnc23)cn2cc(C)nc12 ; "C63"]

To a stirred mixture of tert-butyl 4-(8-carbamoylcinnolin-5-yl)piperazine-1-carboxylate (150 mg, 0.420 mmol, 1.0 equiv) and 6-bromo-8-methoxy-2-methylimidazo[1,2-a]pyrazine (152.4 mg, 0.630 mmol, 1.5 equiv) in dioxane (7.5 mL) was added Cs₂CO₃ (410.2 mg, 1.260 mmol, 3.0 equiv), XantPhos (48.6 mg, 0.084 mmol, 0.2 equiv), and Pd₂(dba)₃ (9.8 mg, 0.042 mmol, 0.1 equiv) at room temperature under nitrogen atmosphere. The resulting mixture was stirred for 24 h at 100° C. under nitrogen atmosphere, then concentrated under reduced pressure to give a residue. The residue was purified by silica gel column chromatography, eluted with PE/EA (1:1) to afford tert-butyl 4-[8-({8-methoxy-2-methylimidazo[1,2-a]pyrazin-6-yl}carbamoyl)cinnolin-5-yl]piperazine-1-carboxylate (120 mg, 55%) as a solid. LCMS (ES, m/z):519 [M+H]⁺.

### Synthesis of Compound 177

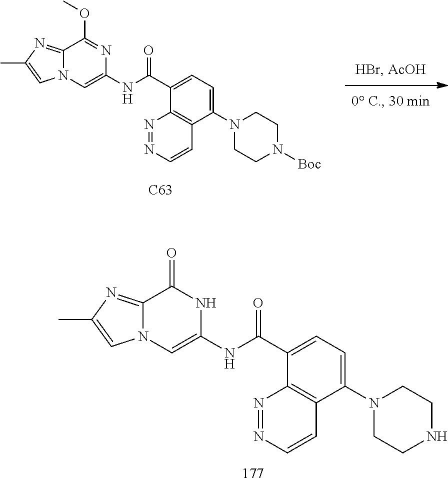 [SMILES: COc1nc(NC(=O)c2ccc(N3CCN(C(=O)OC(C)(C)C)CC3)c3ccnnc23)cn2cc(C)nc12 ; "C63" ; Cc1cn2cc(NC(=O)c3ccc(N4CCNCC4)c4ccnnc34)[nH]c(=O)c2n1 ; "177"]

To tert-butyl 4-[8-({8-methoxy-2-methylimidazo[1,2-a]pyrazin-6-yl}carbamoyl)cinnolin-5-yl]piperazine-1-carboxylate (100 mg, 0.193 mmol, 1 equiv) was added HBr (4 mL, 30% in AcOH, w/w %) dropwise at 0° C. The resulting mixture was stirred for 30 min at 0° C., then concentrated under reduced pressure to give a residue. The residue was purified by Prep-HPLC (Condition 2, Gradient 3) to afford N-{2-methyl-8-oxo-7H-imidazo[1,2-a]pyrazin-6-yl}-5-(piperazin-1-yl)cinnoline-8-carboxamide trifluoroacetic acid salt (19.3 mg, 25%) as a solid. LCMS (ES, m/z):405 [M+H]⁺. ¹H NMR (400 MHz, DMSO-d₆) δ 8.76 (d, J=6.0 Hz, 1H), 8.11 (d, J=8.1 Hz, 1H), 7.79 (d, J=5.8 Hz, 2H), 7.12 (s, 1H), 6.86 (d, J=8.1 Hz, 1H), 2.85-2.77 (m, 4H), 2.76-2.69 (m, 4H), 1.74 (s, 3H).

### Example 74: Synthesis of Compound 179

### Synthesis of Compound 179

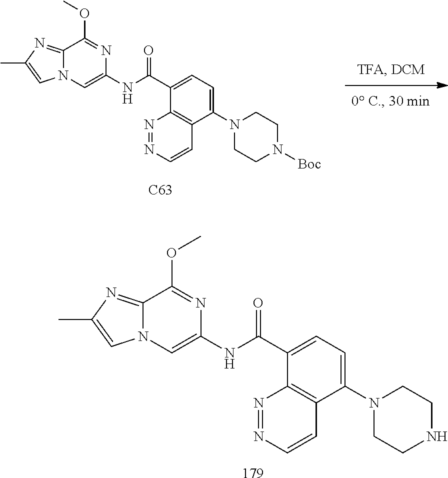

To a stirred solution of tert-butyl 4-[8-({8-methoxy-2-methylimidazo[1,2-a]pyrazin-6-yl}carbamoyl)cinnolin-5-yl]piperazine-1-carboxylate (50 mg, 0.096 mmol, 1.0 equiv) in DCM (1.2 mL) was added TFA (0.4 mL) dropwise at 0° C. The resulting mixture was stirred for 30 min at 0° C., then concentrated under reduced pressure to give a residue. The residue was purified by Prep-HPLC (Condition 2, Gradient 3) to afford N-{8-methoxy-2-methylimidazo[1,2-a]pyrazin-6-yl}-5-(piperazin-1-yl)cinnoline-8-carboxamide as a solid. LCMS (ES, m/z):419 [M+H]⁺. ¹H NMR (400 MHz, CD₃OD) δ 9.54 (d, J=5.9 Hz, 1H), 9.29 (s, 1H), 8.92 (d, J=8.1 Hz, 1H), 8.57 (d, J=5.9 Hz, 1H), 8.03 (s, 1H), 7.66 (d, J=8.1 Hz, 1H), 4.33 (s, 3H), 3.60-3.58 (m, 4H), 3.52-3.50 (m, 4H), 2.55 (s, 3H).

### Example 75: Synthesis of Compound 172

### Synthesis of Intermediate C64

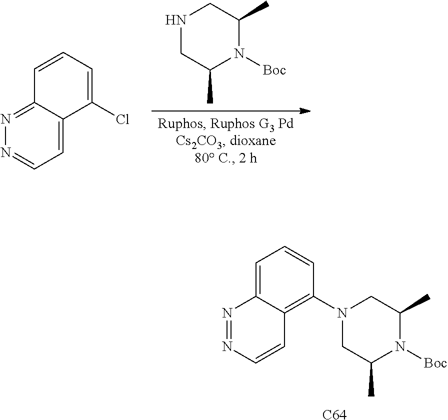

To a stirred mixture of 5-chlorocinnoline (220 mg, 1.337 mmol, 1.0 equiv) and tert-butyl (2R,6S)-2,6-dimethylpiperazine-1-carboxylate (429.7 mg, 2.006 mmol, 1.5 equiv) in dioxane (5.5 mL) was added Cs₂CO₃ (1.31 g, 4.011 mmol, 3 equiv), Ruphos (124.7 mg, 0.267 mmol, 0.2 equiv), and RuPhos Palladacycle Gen.3 (111.8 mg, 0.134 mmol, 0.1 equiv) at room temperature under nitrogen atmosphere. The resulting mixture was stirred for 2 h at 80° C. under nitrogen atmosphere, then concentrated under reduced pressure to give a residue. The residue was purified by silica gel column chromatography, eluted with PE/EA (1:1) to afford tert-butyl (2R,6S)-4-(cinnolin-5-yl)-2,6-dimethylpiperazine-1-carboxylate (330 mg, 72%) as a solid. LCMS (ES, m/z):343 [M+H]⁺.

### Synthesis of Intermediate C65

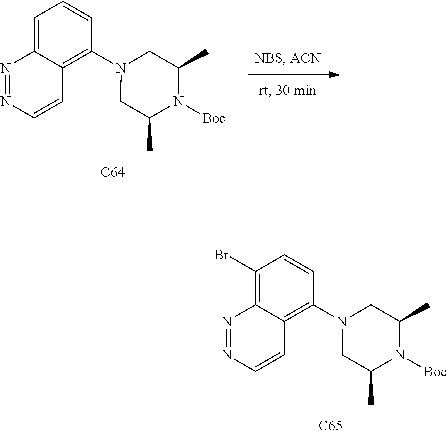 [SMILES: C[C@@H]1CN(c2cccc3nnccc23)C[C@H](C)N1C(=O)OC(C)(C)C ; "C64" ; C[C@@H]1CN(c2ccc(Br)c3nnccc23)C[C@H](C)N1C(=O)OC(C)(C)C ; "C65"]

To a stirred solution of tert-butyl (2R,6S)-4-(cinnolin-5-yl)-2,6-dimethylpiperazine-1-carboxylate (300 mg, 0.876 mmol, 1.0 equiv) in acetonitrile (6 mL) was added NBS (155.9 mg, 0.876 mmol, 1.0 equiv) at room temperature. The resulting mixture was stirred for 30 min at room temperature, then concentrated under reduced pressure, diluted with water (10 mL), and extracted with ethyl acetate (3×10 mL). The organic layers were combined, washed with water (3×5 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl (2R,6S)-4-(8-bromocinnolin-5-yl)-2,6-dimethylpiperazine-1-carboxylate (260 mg, 70%) as a solid. LCMS (ES, m/z):421 [M+H]⁺.

### Synthesis of Intermediate C66

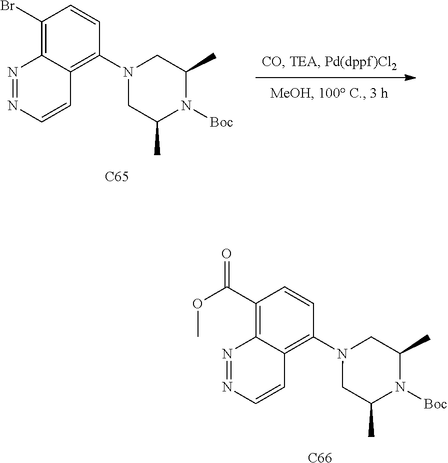 [SMILES: C[C@@H]1CN(c2ccc(Br)c3nnccc23)C[C@H](C)N1C(=O)OC(C)(C)C ; "C65" ; COC(=O)c1ccc(N2C[C@@H](C)N(C(=O)OC(C)(C)C)[C@@H](C)C2)c2ccnnc12 ; "C66"]

To a solution of tert-butyl (2R,6S)-4-(8-bromocinnolin-5-yl)-2,6-dimethylpiperazine-1-carboxylate (230 mg, 0.546 mmol, 1.0 equiv) in methanol (15 mL) was added TEA (165.3 mg, 1.638 mmol, 3.0 eq) and Pd(dppf)Cl₂ (39.9 mg, 0.055 mmol, 0.1 equiv) in a pressure tank. The reaction mixture was pressurized to 1 MPa with carbon monoxide at 100° C. for 3 h. The reaction mixture was cooled to room temperature, then filtered to remove insoluble solids. The resulting mixture was concentrated under reduced pressure to give a residue. The residue was purified by silica gel column chromatography, eluted with PE/EA (1:1) to afford methyl 5-[(3R,5S)-4-(tert-butoxycarbonyl)-3,5-dimethylpiperazin-1-yl]cinnoline-8-carboxylate (200 mg, 91%) as a solid. LCMS (ES, m/z):401 [M+H]⁺.

### Synthesis of Intermediate C67

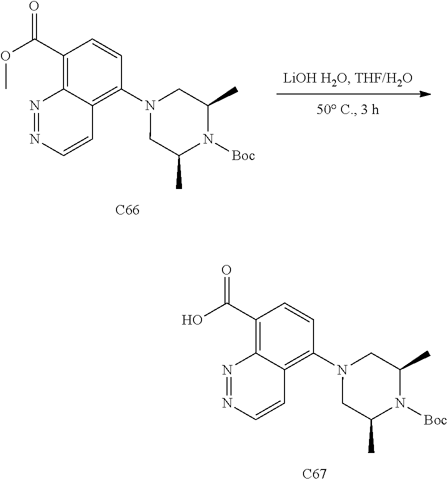 [SMILES: COC(=O)c1ccc(N2C[C@@H](C)N(C(=O)OC(C)(C)C)[C@@H](C)C2)c2ccnnc12 ; "C66" ; C[C@@H]1CN(c2ccc(C(=O)O)c3nnccc23)C[C@H](C)N1C(=O)OC(C)(C)C ; "C67"]

To a stirred mixture of methyl 5-[(3R,5S)-4-(tert-butoxycarbonyl)-3,5-dimethylpiperazin-1-yl]cinnoline-8-carboxylate (200 mg, 0.499 mmol, 1 equiv) in THE (2.5 mL) and water (2.5 mL) was added lithiumol hydrate (167.6 mg, 3.992 mmol, 8.0 equiv) in portions at room temperature. The resulting mixture was stirred for 3 h at 50° C., then diluted with water (10 mL), acidified to pH 6 with concentrated HCl, and extracted with ethyl acetate (3×10 mL). The organic layers were combined, washed with water (3×10 mL), dried over anhydrous Na₂SO₄, and filtered. After filtration, the filtrate was concentrated under reduced pressure to afford 5-[(3R,5S)-4-(tert-butoxycarbonyl)-3,5-dimethylpiperazin-1-yl]cinnoline-8-carboxylic acid (200 mg, 98%) as a solid. LCMS (ES, m/z):387 [M+H]⁺.

### Synthesis of Intermediate C68

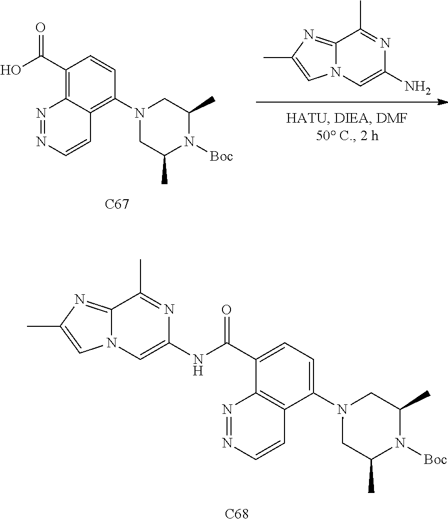 [SMILES: C[C@@H]1CN(c2ccc(C(=O)O)c3nnccc23)C[C@H](C)N1C(=O)OC(C)(C)C ; Cc1cn2cc(N)nc(C)c2n1 ; "C67" ; Cc1cn2cc(NC(=O)c3ccc(N4C[C@@H](C)N(C(=O)OC(C)(C)C)[C@@H](C)C4)c4ccnnc34)nc(C)c2n1 ; "C68"]

To a stirred mixture of 5-[(3R,5S)-4-(tert-butoxycarbonyl)-3,5-dimethylpiperazin-1-yl]cinnoline-8-carboxylic acid (100 mg, 0.259 mmol, 1.0 equiv) and HATU (177.1 mg, 0.466 mmol, 1.8 equiv) in DMF (2.5 mL) was added DIEA (133.8 mg, 1.036 mmol, 4.0 equiv) and 2,8-dimethylimidazo[1,2-a]pyrazin-6-amine (62.9 mg, 0.389 mmol, 1.5 equiv) at room temperature under nitrogen atmosphere. The resulting mixture was stirred for 2 h at 50° C. under nitrogen atmosphere, then concentrated under reduced pressure to give a residue. The residue was purified by silica gel column chromatography, eluted with PE/EA (1:1) to afford rac-tert-butyl (2R,6S)-4-[8-({2,8-dimethylimidazo[1,2-a]pyrazin-6-yl}carbamoyl)cinnolin-5-yl]-2,6-dimethylpiperazine-1-carboxylate (130 mg, 95%) as a solid. LCMS (ES, m/z):531 [M+H]⁺.

### Synthesis of Compound 172

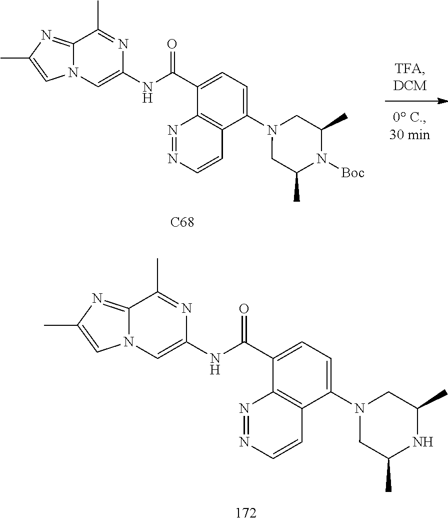

To a stirred solution of tert-butyl (2R,6S)-4-[8-({2,8-dimethylimidazo[1,2-a]pyrazin-6-yl}carbamoyl)cinnolin-5-yl]-2,6-dimethylpiperazine-1-carboxylate (80 mg, 0.151 mmol, 1.0 equiv) in DCM (0.9 mL) was added TFA (0.3 mL) dropwise at 0° C. The resulting mixture was stirred for 30 min at 0° C., then concentrated under vacuum to give a residue. The residue was purified by Prep-HPLC (Condition 1, Gradient 2) to afford N-{2,8-dimethylimidazo[1,2-a]pyrazin-6-yl}-5-[(3R,5S)-3,5-dimethylpiperazin-1-yl]cinnoline-8-carboxamide (27.8 mg, 43%) as a solid. LCMS (ES, m/z):431 [M+H]⁺. ¹H NMR (300 MHz, DMSO-d₆) δ 13.18 (s, 1H), 9.53 (d, J=5.9 Hz, 1H), 9.36 (s, 1H), 8.74 (d, J=8.1 Hz, 1H), 8.39 (d, J=5.9 Hz, 1H), 8.02 (s, 1H), 7.46 (d, J=8.2 Hz, 1H), 3.39-3.38 (m, 2H), 3.14-3.12 (m, 2H), 2.74 (s, 3H), 2.46-2.42 (m, 2H), 2.40 (s, 3H), 1.04 (d, J=6.3 Hz, 6H).

### Example 76: Synthesis of Compound 180

### Synthesis of Intermediate C69

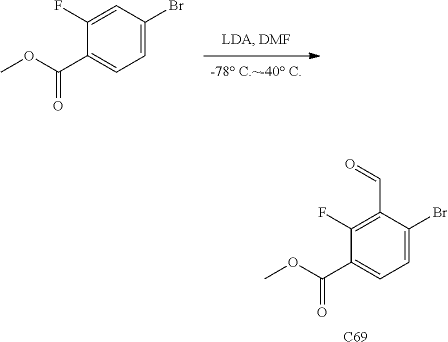

To a solution of methyl 4-bromo-2-fluorobenzoate (3 g, 12.874 mmol, 1 equiv) in THE (10 mL) was added LDA (in 2 M THF) (1.79 g, 16.736 mmol, 1.3 equiv) dropwise at −70° C. under N₂ atmosphere. The reaction mixture was stirred at −70° C. for 30 min. A solution of DMF (1.88 g, 25.748 mmol, 2.0 equiv) in 5 mL THE was added dropwise to the reaction mixture, and the resulting mixture was stirred for another 60 mins at −70° C. The reaction mixture was quenched with saturated NH₄Cl (50 mL) and extracted with ethyl acetate (2×15 mL). The organic extracts were combined, washed with brine (50 mL), dried over anhydrous Na₂SO₄, filtered, and the filtrate concentrated under vacuum to give a residue. The residue was purified by silica gel column chromatography, eluted with PE/EA (2:1) to afford methyl 4-bromo-2-fluoro-3-formylbenzoate (2 g, 60%) as a solid.

### Synthesis of Intermediate C70

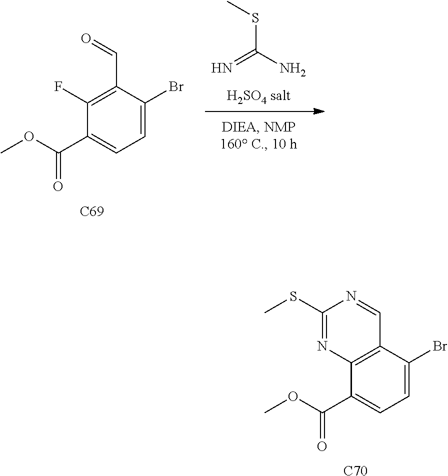

A mixture of methyl 4-bromo-2-fluoro-3-formylbenzoate (100 mg, 0.383 mmol, 1 equiv), methylsulfanylmethanimidamide (207.18 mg, 2.298 mmol, 6 equiv), NMP (5 mL), and DIEA (297.07 mg, 2.298 mmol, 6 equiv) was heated for 10 h at 160° C. under microwave. The reaction mixture was cooled to room temperature, then purified by reverse phase flash (Condition 3, Gradient 1) to afford methyl 5-bromo-2-(methylsulfanyl)quinazoline-8-carboxylate (25 mg, 21%) as a solid. LCMS (ES, m/z): 313 [M+H]⁺.

### Synthesis of Intermediate C71

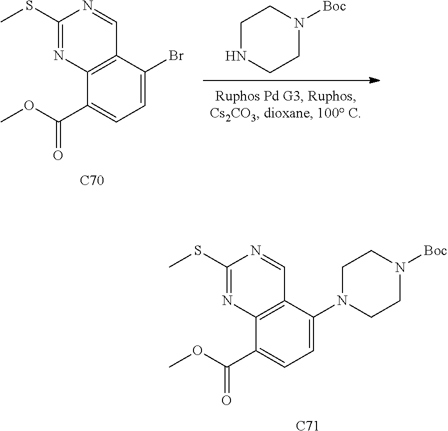 [SMILES: COC(=O)c1ccc(Br)c2cnc(SC)nc12 ; CC(C)(C)OC(=O)N1CCNCC1 ; "C70" ; COC(=O)c1ccc(N2CCN(C(=O)OC(C)(C)C)CC2)c2cnc(SC)nc12 ; "C71"]

To a mixture of methyl 5-bromo-2-(methylsulfanyl)quinazoline-8-carboxylate (250 mg, 0.798 mmol, 1 equiv), tert-butyl piperazine-1-carboxylate (223.03 mg, 1.197 mmol, 1.5 equiv), and Cs₂CO₃ (520.20 mg, 1.596 mmol, 2 equiv) in dioxane (5 mL) was added Ruphos (74.50 mg, 0.160 mmol, 0.2 equiv) and RuPhos Palladacycle Gen.3 (66.77 mg, 0.080 mmol, 0.1 equiv). The reaction mixture was stirred for 4 h at 100° C. under a nitrogen atmosphere, then concentrated under reduced pressure to give a residue. The residue was purified by silica gel column chromatography, eluted with PE/EA (1:1) to afford methyl 5-[4-(tert-butoxycarbonyl)piperazin-1-yl]-2-(methylsulfanyl)quinazoline-8-carboxylate (150 mg, 45%) as a solid. LCMS (ES, m/z): 419 [M+H]⁺.

### Synthesis of Intermediate C72

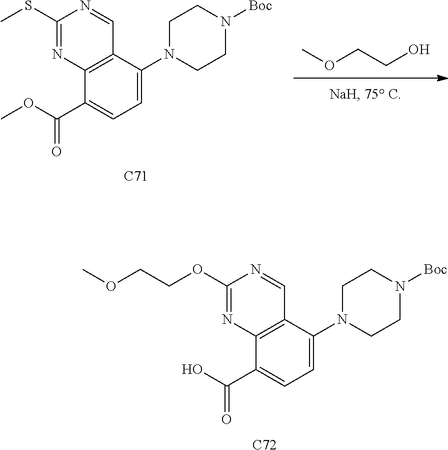

To 2-methoxyethanol (5 mL, 65.707 mmol, 196.42 equiv) was added sodium hydride (60% in oil, 17.93 mg) at 0° C. The reaction mixture was stirred for 15 min, then methyl 5-[4-(tert-butoxycarbonyl)piperazin-1-yl]-2-(methylsulfanyl)quinazoline-8-carboxylate (140 mg, 0.335 mmol, 1 equiv) was added. The reaction mixture was heated to 75° C. and stirred for an additional 2 h. The reaction mixture was quenched with water, acidified to pH 4 with HCl (aq.), then concentrated under reduced pressure to give a residue. The residue was purified by reverse phase flash (Condition 3, Gradient 2) to afford 5-[4-(tert-butoxycarbonyl)piperazin-1-yl]-2-(2-methoxyethoxy)quinazoline-8-carboxylic acid (90 mg, 62%) as a solid. LCMS (ES, m/z): 433 [M+H]⁺.

### Synthesis of Intermediate C73

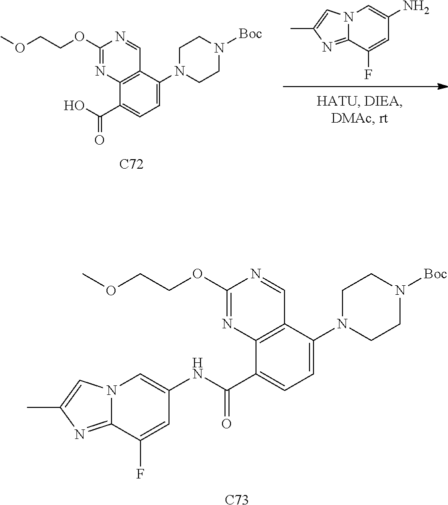 [SMILES: COCCOc1ncc2c(N3CCN(C(=O)OC(C)(C)C)CC3)ccc(C(=O)O)c2n1 ; Cc1cn2cc(N)cc(F)c2n1 ; "C72" ; COCCOc1ncc2c(N3CCN(C(=O)OC(C)(C)C)CC3)ccc(C(=O)Nc3cc(F)c4nc(C)cn4c3)c2n1 ; "C73"]

A mixture of 5-[4-(tert-butoxycarbonyl)piperazin-1-yl]-2-(2-methoxyethoxy)quinazoline-8-carboxylic acid (80 mg, 0.185 mmol, 1 equiv), HATU (84.40 mg, 0.222 mmol, 1.2 equiv), DIEA (47.82 mg, 0.370 mmol, 2.0 equiv), and 8-fluoro-2-methylimidazo[1,2-a]pyridin-6-amine (36.66 mg, 0.222 mmol, 1.2 equiv) in DMF (5 mL, 64.608 mmol, 349.27 equiv) was stirred for 2 h at room temperature, then concentrated under reduced pressure to give a residue. The residue was purified by reverse flash chromatography (Condition 3, Gradient 3) to afford tert-butyl 4-[8-({8-fluoro-2-methylimidazo[1,2-a]pyridin-6-yl}carbamoyl)-2-(2-methoxyethoxy)quinazolin-5-yl]piperazine-1-carboxylate (50 mg, 47%) as a solid. LCMS (ES, m/z): 580 [M+H]⁺.

### Synthesis of Compound 180

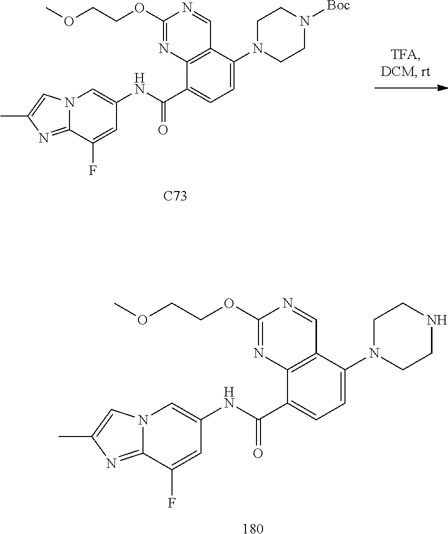 [SMILES: COCCOc1ncc2c(N3CCN(C(=O)OC(C)(C)C)CC3)ccc(C(=O)Nc3cc(F)c4nc(C)cn4c3)c2n1 ; "C73" ; COCCOc1ncc2c(N3CCNCC3)ccc(C(=O)Nc3cc(F)c4nc(C)cn4c3)c2n1 ; "180"]

A solution of tert-butyl 4-[8-({8-fluoro-2-methylimidazo[1,2-a]pyridin-6-yl}carbamoyl)-2-(2-methoxyethoxy)quinazolin-5-yl]piperazine-1-carboxylate (40 mg, 0.069 mmol, 1 equiv) in DCM was treated with TFA (0.5 mL, 6.732 mmol, 97.55 equiv) for 1 h at room temperature. The resulting mixture was concentrated under reduced pressure to give a residue. The residue was purified by Prep-HPLC (Condition 4, Gradient 1) to afford N-{8-fluoro-2-methylimidazo[1,2-a]pyridin-6-yl}-2-(2-methoxyethoxy)-5-(piperazin-1-yl)quinazoline-8-carboxamide; trifluoroacetic acid (2.6 mg, 6%) as a solid. LCMS (ES, m/z): 480 [M+H]⁺. ¹H NMR (400 MHz, DMSO-d₆) δ 11.73 (s, 1H), 9.67 (s, 1H), 9.27 (s, 1H), 8.88 (s, 2H), 8.58 (d, J=8.3 Hz, 1H), 8.02 (s, 1H), 7.36 (d, J=12.3 Hz, 1H), 7.30 (d, J=8.4 Hz, 1H), 4.75 (t, 2H), 3.84 (t, 2H), 3.43-3.32 (m, 11H). ¹⁹F NMR (300 MHz, DMSO-d₆) δ-73.79, −131.17

### Example 77: Synthesis of Compound 173

### Synthesis of Intermediate C74

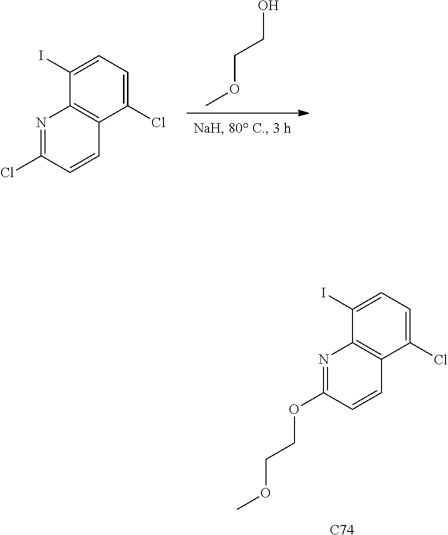

To 2-methoxyethanol (3.76 g, 49.376 mmol, 32 equiv) was added NaH (0.19 g, 4.629 mmol, 3.0 equiv, 60%) in portions at 0° C. The resulting mixture was stirred for 0.5 h at 0° C. To the reaction mixture was added 2,5-dichloro-8-iodoquinoline (0.5 g, 1.543 mmol, 1 equiv) in portions at 0° C. The resulting mixture was stirred for an additional 3 h at room temperature, then diluted with water (10 mL). A solid precipitated that was collected by filtration and washed with water (2×5 mL) to afford 5-chloro-8-iodo-2-(2-methoxyethoxy)quinolone (4.0 g). LCMS (ES, m/z): 364 [M+H]⁺.

### Synthesis of Intermediate C75

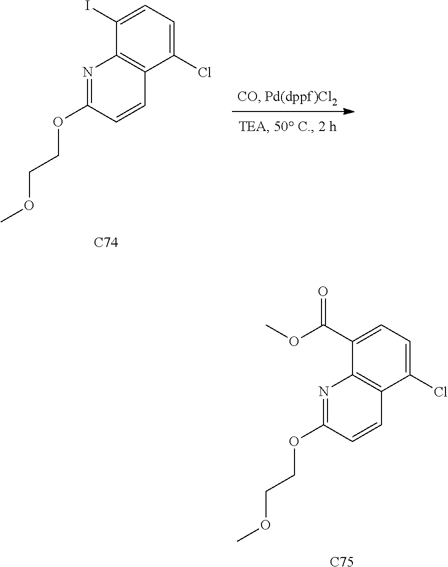 [SMILES: COCCOc1ccc2c(Cl)ccc(I)c2n1 ; "C74" ; COCCOc1ccc2c(Cl)ccc(C(=O)OC)c2n1 ; "C75"]

To a solution of 5-chloro-8-iodo-2-(2-methoxyethoxy)quinolone (300 mg, 0.825 mmol, 1 equiv) in methanol (10 mL) was added Pd(dppf)Cl₂·CH₂Cl₂ (67.22 mg, 0.083 mmol, 0.1 equiv) and TEA (250.48 mg, 2.475 mmol, 3.0 equiv) in a pressure tank. The reaction mixture was purged with nitrogen for 1 min, then pressurized to 20 atm with carbon monoxide and heated at 50° C. for 2 h. The reaction mixture was cooled to room temperature, then filtered to remove insoluble solids. The filtrate was concentrated under reduced pressure to give a residue. The residue was purified by silica gel column chromatography, eluted with PE:EA (4:1) to afford methyl 5-chloro-2-(2-methoxyethoxy)quinoline-8-carboxylate (230 mg, 94%) as a solid. LCMS (ES, m/z): 296 [M+H]⁺.

### Synthesis of Intermediate C76

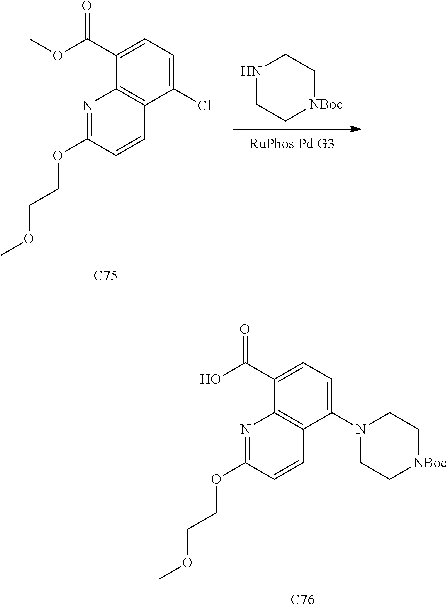 [SMILES: COCCOc1ccc2c(Cl)ccc(C(=O)OC)c2n1 ; CC(C)(C)OC(=O)N1CCNCC1 ; "C75" ; COCCOc1ccc2c(N3CCN(C(=O)OC(C)(C)C)CC3)ccc(C(=O)O)c2n1 ; "C76"]

To a mixture of methyl 5-chloro-2-(2-methoxyethoxy)quinoline-8-carboxylate (200 mg, 0.676 mmol, 1 equiv), tert-butyl piperazine-1-carboxylate (251.93 mg, 1.352 mmol, 2.0 equiv), and t-BuONa (129.99 mg, 1.352 mmol, 2.0 equiv) in DMF (6 mL) was added and RuPhos Pd G3 (56.56 mg, 0.068 mmol, 0.1 equiv) and RuPhos (63.12 mg, 0.135 mmol, 0.2 equiv). The reaction mixture was stirred for 2 h at 110° C. under a nitrogen atmosphere, then filtered. The filtrate was purified by reverse flash chromatography (Condition 1, Gradient 4) to afford methyl 5-[4-(tert-butoxycarbonyl)piperazin-1-yl]-2-(2-methoxyethoxy)quinoline-8-carboxylate (40 mg, 13%) as a solid. LCMS (ES, m/z): 432 [M+H]⁺.

### Synthesis of Intermediate C77

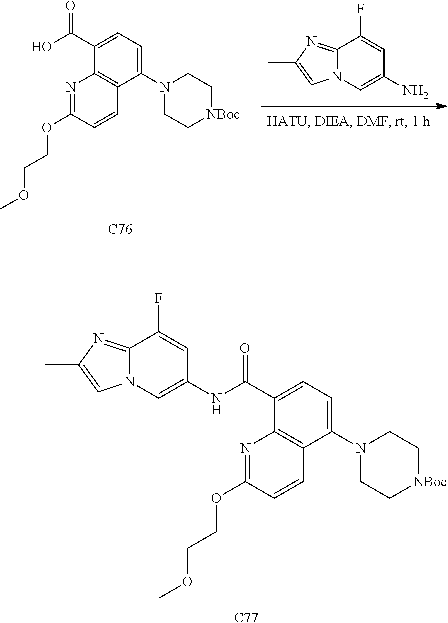 [SMILES: COCCOc1ccc2c(N3CCN(C(=O)OC(C)(C)C)CC3)ccc(C(=O)O)c2n1 ; Cc1cn2cc(N)cc(F)c2n1 ; "C76" ; COCCOc1ccc2c(N3CCN(C(=O)OC(C)(C)C)CC3)ccc(C(=O)Nc3cc(F)c4nc(C)cn4c3)c2n1 ; "C77"]

To a stirred mixture of 5-[4-(tert-butoxycarbonyl)piperazin-1-yl]-2-(2-methoxyethoxy)quinoline-8-carboxylic acid (35 mg, 0.081 mmol, 1 equiv) and HATU (37.01 mg, 0.097 mmol, 1.2 equiv) in DMF (2 mL) was added DIEA (31.45 mg, 0.243 mmol, 3.0 equiv) and 8-fluoro-2-methylimidazo[1,2-a]pyridin-6-amine (16.08 mg, 0.097 mmol, 1.2 equiv) in portions at room temperature. The resulting mixture was stirred for 1 h at room temperature, then purified by reverse flash chromatography (Condition 2, Gradient 2) to afford tert-butyl 4-[8-({8-fluoro-2-methylimidazo[1,2-a]pyridin-6-yl}carbamoyl)-2-(2-methoxyethoxy)quinolin-5-yl]piperazine-1-carboxylate (40 mg, 85%) as a solid. LCMS (ES, m/z): 579 [M+H]⁺.

### Synthesis of Compound 173

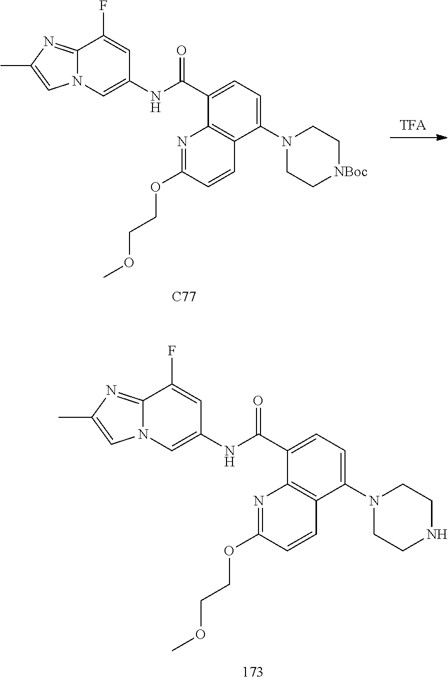 [SMILES: COCCOc1ccc2c(N3CCN(C(=O)OC(C)(C)C)CC3)ccc(C(=O)Nc3cc(F)c4nc(C)cn4c3)c2n1 ; "TFA" ; "C77" ; COCCOc1ccc2c(N3CCNCC3)ccc(C(=O)Nc3cc(F)c4nc(C)cn4c3)c2n1 ; "173"]

A mixture of tert-butyl 4-[8-({8-fluoro-2-methylimidazo[1,2-a]pyridin-6-yl}carbamoyl)-2-(2-methoxyethoxy)quinolin-5-yl]piperazine-1-carboxylate (38 mg, 0.066 mmol, 1 equiv) and TFA (0.1 mL) in DCM (2 mL) was stirred for 1 h at room temperature. The resulting mixture was basified to pH 8 with 7 N NH₃ (gas) in methanol, then concentrated under reduced pressure to give a residue. The residue was purified by reverse flash chromatography (Condition 1, Gradient 5) to afford N-{8-fluoro-2-methylimidazo[1,2-a]pyridin-6-yl}-2-(2-methoxyethoxy)-5-(piperazin-1-yl)quinoline-8-carboxamide (17 mg, 54%) as a solid. LCMS (ES, m/z): 479 [M+H]⁺. ¹H NMR (300 MHz, DMSO-d₆) δ 11.88 (s, 1H), 9.20 (d, J=1.6 Hz, 1H), 8.48 (d, J=9.2 Hz, 1H), 8.40 (d, J=8.2 Hz, 1H), 7.92 (dd, J=3.2, 1.0 Hz, 1H), 7.26 (dd, J=12.6, 1.7 Hz, 1H), 7.18 (dd, J=8.7, 4.8 Hz, 2H), 4.72-4.63 (m, 2H), 3.83-3.73 (m, 2H), 3.06-2.95 (m, 8H), 2.36 (d, J=0.9 Hz, 3H).

### Example 78: Synthesis of Compound 181

### Synthesis of Intermediate C78

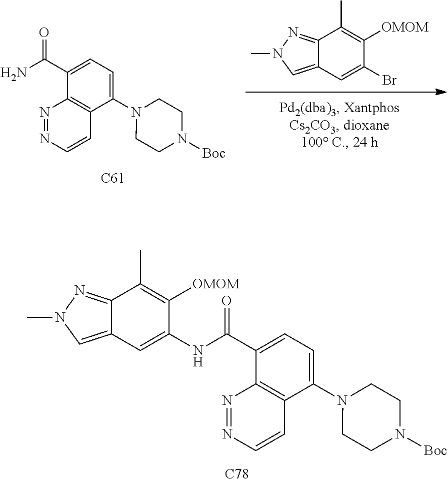

To a stirred mixture of tert-butyl 4-(8-carbamoylcinnolin-5-yl)piperazine-1-carboxylate (70 mg, 0.196 mmol, 1.0 equiv) and 5-bromo-6-(methoxymethoxy)-2,7-dimethylindazole (83.77 mg, 0.294 mmol, 1.5 equiv) in dioxane (3.5 mL) was added Cs₂CO₃ (191.4 mg, 0.588 mmol, 3 equiv), Xantphos (22.7 mg, 0.039 mmol, 0.2 equiv), and Pd₂(dba)₃ (17.9 mg, 0.020 mmol, 0.1 equiv) at room temperature under nitrogen atmosphere. The resulting mixture was stirred for 24 h at 100° C. under nitrogen atmosphere, then concentrated under vacuum to give a residue. The residue was purified by silica gel column chromatography, eluted with EA (100%) to afford tert-butyl 4-(8-{[6-(methoxymethoxy)-2,7-dimethylindazol-5-yl]carbamoyl}cinnolin-5-yl)piperazine-1-carboxylate (80 mg, 73%) as a solid. LCMS (ES, m/z):562 [M+H]⁺.

### Synthesis of Compound 181

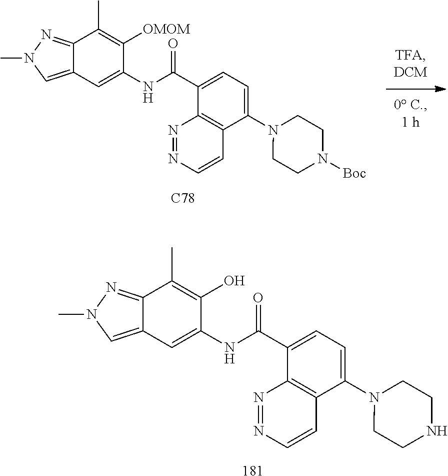

To a stirred solution of tert-butyl 4-(8-{[6-(methoxymethoxy)-2,7-dimethylindazol-5-yl]carbamoyl}cinnolin-5-yl)piperazine-1-carboxylate (60 mg, 0.107 mmol, 1.0 equiv) in DCM (1.8 mL) was added TFA (0.6 mL) dropwise at 0° C. The resulting mixture was stirred for 1 h at 0° C., then concentrated under reduced pressure to give a residue. The residue was purified by Prep-HPLC (Condition 2, Gradient 3) to afford N-(6-hydroxy-2,7-dimethylindazol-5-yl)-5-(piperazin-1-yl)cinnoline-8-carboxamide (30 mg, 67%) as a solid. LCMS (ES, m/z):418 [M+H]⁺. ¹H NMR (400 MHz, DMSO-d₆) δ 13.28 (s, 1H), 9.56 (d, J=5.9 Hz, 1H), 9.36 (s, 1H), 8.99-8.93 (m, 2H), 8.81 (d, J=8.1 Hz, 1H), 8.69 (s, 1H), 8.51 (d, J=6.0 Hz, 1H), 8.18 (s, 1H), 7.62 (d, J=8.2 Hz, 1H), 4.10 (s, 3H), 3.46-3.45 (m, 4H), 3.42-3.35 (m, 4H), 2.42 (s, 3H).

### Example 79: Synthesis of Compound 182

### Synthesis of Compound 182

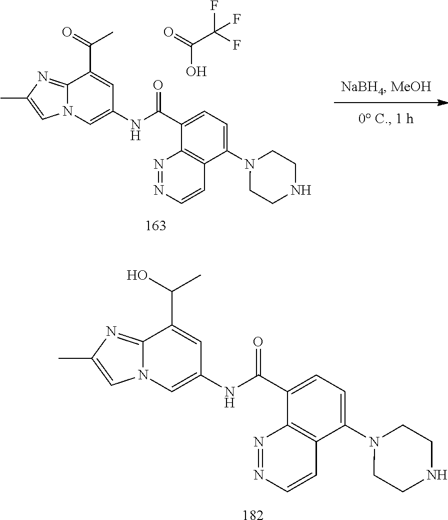

To a stirred solution N-{8-acetyl-2-methylimidazo[1,2-a]pyridin-6-yl}-5(piperazin-1-yl)cinnoline-8-carboxamide (23 mg, 0.054 mmol, 1 equiv) in methanol (0.5 mL) was added NaBH₄ (2.1 mg, 0.054 mmol, 1.0 equiv) at 0° C. The resulting mixture was stirred for 1 h at 0° C., then quenched with water at 0° C. The resulting mixture was extracted with ethyl acetate (3×3 mL). The organic layers were combined, dried over anhydrous Na₂SO₄, and filtered. After filtration, the filtrate was concentrated under reduced pressure to give a residue. The residue was purified by Prep-HPLC (Condition 2, Gradient 3) to afford N-[8-(1-hydroxyethyl)-2-methylimidazo[1,2-a]pyridin-6-yl]-5-(piperazin-1-yl)cinnoline-8-carboxamide trifluoroacetate (10.7 mg, 46%) as a solid. LCMS (ES, m/z):244.0 [M+H]⁺.1H NMR (400 MHz, CD₃OD) δ 9.72 (d, J=1.9 Hz, 1H), 9.55 (d, J=6.0 Hz, 1H), 8.89 (d, J=8.1 Hz, 1H), 8.59 (d, J=6.0 Hz, 1H), 8.06 (dd, J=8.3, 1.7 Hz, 2H), 7.67 (d, J=8.1 Hz, 1H), 5.30 (q, J=6.1 Hz, 1H), 3.62-3.60 (m, 4H), 3.53-3.51 (m, 4H), 2.59 (d, J=1.1 Hz, 3H), 1.68 (d, J=6.5 Hz, 3H).

### Example 80: Synthesis of Compound 185

### Synthesis of Intermediate B79

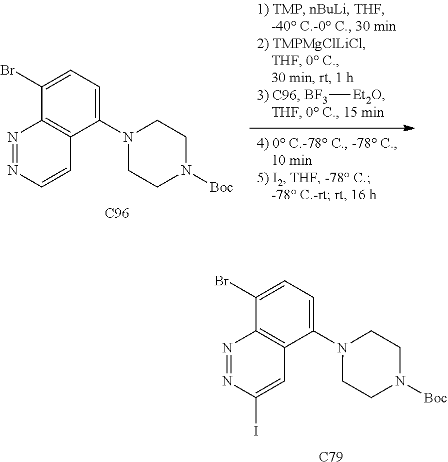

In a nitrogen flushed Schlenk flask, 2,2,6,6-tetramethylpiperidine (0.79 g, 5.594 mmol, 1.1 equiv) was dissolved in THF (16 mL). The resulting solution was cooled to −40° C. To the solution, n-BuLi (2.24 mL, 5.594 mmol, 1.1 equiv) was added dropwise. The reaction mixture was warmed to 0° C., then stirred for 30 min. A solution of 2,2,6,6-tetramethylpiperidinylmagnesium chloride lithium chloride complex in THE (1M) (5.6 mL, 5.594 mmol, 1.1 equiv) was then added dropwise to the reaction mixture. The reaction mixture was stirred at 0° C. for an additional 30 min, warmed to room temperature, and stirred for 1 h. A dry nitrogen-flushed Schlenk flask was charged with tert-butyl 4-(8-bromocinnolin-5-yl)piperazine-1-carboxylate (2.00 g, 5.085 mmol, 1.0 equiv) and dry THE (40 mL). The resulting solution was cooled to 0° C., and dry BF₃·EtO (0.79 g, 5.594 mmol, 1.1 equiv) was added dropwise. The resulting mixture was stirred for 15 min, cooled to −78° C., and the TMP₂Mg 2·LiCl solution was added dropwise. The reaction mixture was stirred for an additional 10 min, then to the mixture was added I₂ (2.58 g, 10.170 mmol, 2.0 equiv) at −78° C. The resulting mixture was warmed to room temperature and stirred for an additional 16 h at room temperature. The reaction mixture was quenched with NH₄Cl (aq.) (10 mL) at 0° C., diluted with water (100 mL), and extracted with ethyl acetate (2×100 mL). The organic layers were combined, washed with Na₂SO₃ (aq.) (1×200 mL) and water (1×200 mL), dried over anhydrous Na₂SO₄, and filtered. After filtration, the filtrate was concentrated under reduced pressure to give a residue. The residue was purified by silica gel column chromatography, eluted with PE/EA (3:1) to afford tert-butyl 4-(8-bromo-3-iodocinnolin-5-yl)piperazine-1-carboxylate (800 mg, 30%) as a solid. LCMS (ES, m/z):519 [M+H]⁺.

### Synthesis of Intermediate C80

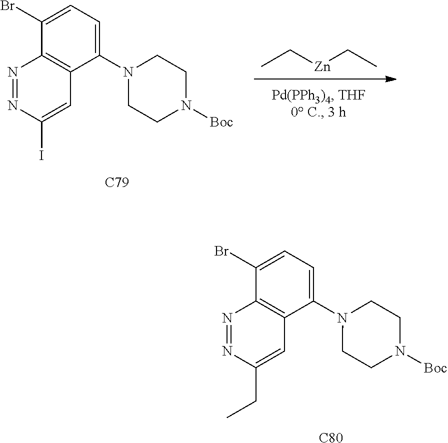

To a solution of tert-butyl 4-(8-bromo-3-iodocinnolin-5-yl)piperazine-1-carboxylate (130.0 mg, 0.251 mmol, 1.0 equiv) in THE (2.6 mL) was added Pd(PPh₃)₄(28.8 mg, 0.025 mmol, 0.1 equiv) and diethylzinc (0.38 mL, 0.376 mmol, 1.5 equiv) (1M in THF) under nitrogen atmosphere. The resulting mixture was stirred for 2 h at 0° C., then quenched with NH₄Cl (aq.) (3 mL). The resulting mixture was extracted with ethyl acetate (2×3 mL). The organic layers were combined, washed with water (1×5 mL), dried over anhydrous Na₂SO₄, and filtered. After filtration, the filtrate was concentrated under reduced pressure to give a residue. The residue was purified by silica gel column chromatography, eluted with PE/EA (3:1) to afford tert-butyl 4-(8-bromo-3-ethylcinnolin-5-yl)piperazine-1-carboxylate (75 mg, 71%) as a solid. LCMS (ES, m/z):421 [M+H]⁺.

### Synthesis of Intermediate C81

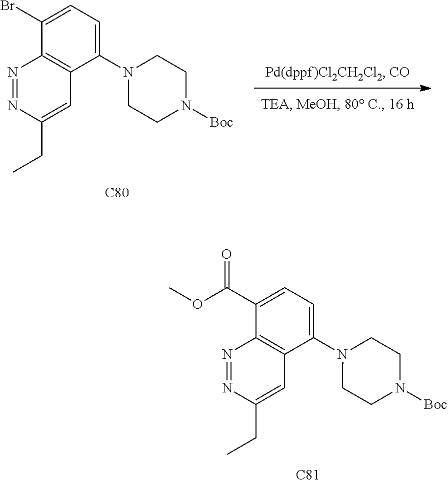 [SMILES: CCc1cc2c(N3CCN(C(=O)OC(C)(C)C)CC3)ccc(Br)c2nn1 ; "C80" ; CCc1cc2c(N3CCN(C(=O)OC(C)(C)C)CC3)ccc(C(=O)OC)c2nn1 ; "C81"]

To a solution of tert-butyl 4-(8-bromo-3-ethylcinnolin-5-yl)piperazine-1-carboxylate (75.0 mg, 0.178 mmol, 1.0 equiv) in methanol (20 mL) was added TEA (54.0 mg, 0.534 mmol, 3.0 equiv) and Pd(dppf)Cl₂CH₂Cl₂ (14.5 mg, 0.018 mmol, 0.1 equiv) in a pressure tank. The reaction mixture was purged with nitrogen for 1 min, then was pressurized to 1 MPa with carbon monoxide at 80° C. for 16 h. The reaction mixture was cooled to room temperature and filtered to remove insoluble solids. The filtrate was concentrated under vacuum to give a residue. The residue was purified by silica gel column chromatography, eluted with PE/EA (3:1) to afford methyl 5-[4-(tert-butoxycarbonyl)piperazin-1-yl]-3-ethylcinnoline-8-carboxylate (70 mg, 98%) as a solid. LCMS (ES, m/z):401 [M+H]⁺.

### Synthesis of Intermediate C82

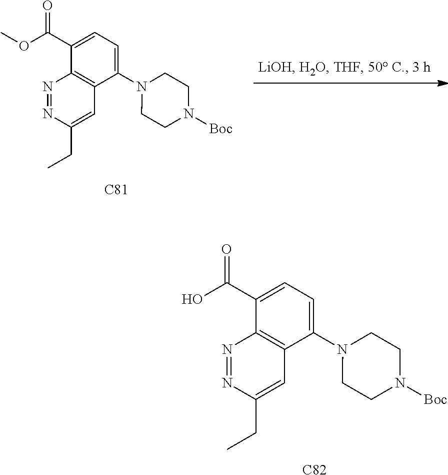 [SMILES: CCc1cc2c(N3CCN(C(=O)OC(C)(C)C)CC3)ccc(C(=O)OC)c2nn1 ; "C81" ; CCc1cc2c(N3CCN(C(=O)OC(C)(C)C)CC3)ccc(C(=O)O)c2nn1 ; "C82"]

To a stirred mixture of methyl 5-[4-(tert-butoxycarbonyl)piperazin-1-yl]-3-ethylcinnoline-8-carboxylate (70.0 mg, 0.175 mmol, 1.0 equiv) in THE (1 mL) was added water (1 mL) and LiOH H₂O (58.7 mg, 1.400 mmol, 8.0 equiv) at room temperature. The resulting mixture was stirred for 3 h at 50° C., then acidified to pH 6 with 1 N of HCl. The resulting mixture was extracted with ethyl acetate (3×2 mL). The organic layers were combined, washed with water (2×2 mL), dried over anhydrous Na₂SO₄, and filtered. After filtration, the filtrate was concentrated under reduced pressure to afford 5-[4-(tert-butoxycarbonyl)piperazin-1-yl]-3-ethylcinnoline-8-carboxylic acid (55 mg, 81%) as a solid. LCMS (ES, m/z):387 [M+H]⁺.

### Synthesis of Intermediate C83

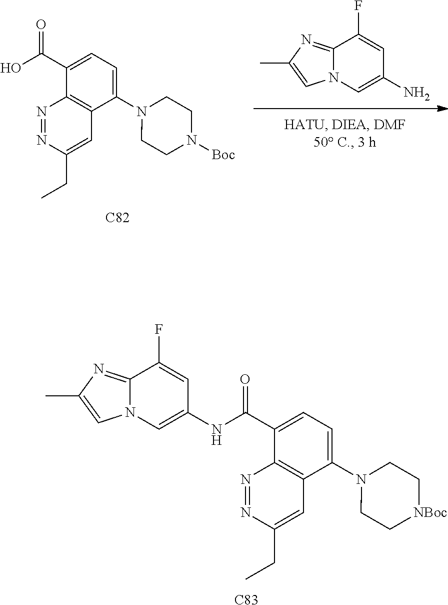 [SMILES: CCc1cc2c(N3CCN(C(=O)OC(C)(C)C)CC3)ccc(C(=O)O)c2nn1 ; Cc1cn2cc(N)cc(F)c2n1 ; "C82" ; CCc1cc2c(N3CCN(C(=O)OC(C)(C)C)CC3)ccc(C(=O)Nc3cc(F)c4nc(C)cn4c3)c2nn1 ; "C83"]

To a stirred mixture of 5-[4-(tert-butoxycarbonyl)piperazin-1-yl]-3-ethylcinnoline-8-carboxylic acid (55 mg, 0.142 mmol, 1.00 equiv) and 8-fluoro-2-methylimidazo[1,2-a]pyridin-6-amine (23.5 mg, 0.142 mmol, 1.00 equiv) in DMF (1.0 mL) was added DIEA (73.6 mg, 0.568 mmol, 4.0 equiv) and HATU (81.2 mg, 0.213 mmol, 1.5 equiv) at room temperature. The resulting mixture was stirred for 3 h at 50° C., then cooled to room temperature. The resulting mixture was diluted with water (3 mL) and extracted with ethyl acetate (3×3 mL). The organic layers were combined, washed with water (3×3 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[3-ethyl-8-({8-fluoro-2-methylimidazo[1,2-a]pyridin-6-yl}carbamoyl)cinnolin-5-yl]piperazine-1-carboxylate (55 mg, 72%) as a solid. LCMS (ES, m/z):534[M+H]⁺.

### Synthesis of Compound 185

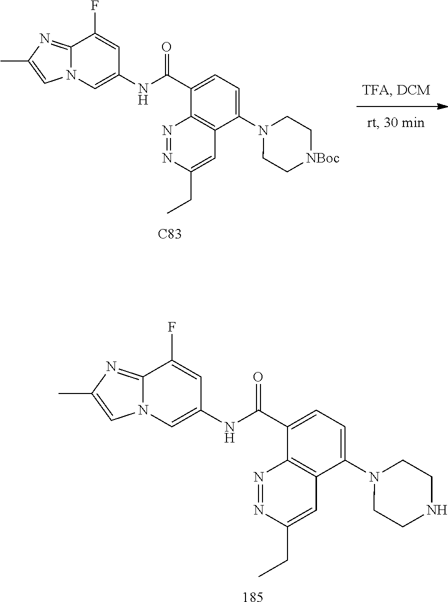

To a stirred mixture of tert-butyl 4-[3-ethyl-8-({8-fluoro-2-methylimidazo[1,2-a]pyridin-6-yl}carbamoyl)cinnolin-5-yl]piperazine-1-carboxylate (60 mg, 0.112 mmol, 1 equiv) in DCM (1 mL) was added TFA (0.25 mL) dropwise at room temperature. The resulting mixture was concentrated under vacuum to give a residue. The residue was purified by Prep-HPLC (Condition 1, Gradient 1) to afford 3-ethyl-N-{8-fluoro-2-methylimidazo[1,2-a]pyridin-6-yl}-5-(piperazin-1-yl)cinnoline-8-carboxamide (14 mg, 29%) as a solid. LCMS (ES, m/z):434 [M+H]⁺. 1H NMR (400 MHz, DMSO-d₆) δ 12.28 (s, 1H), 9.22 (s, 1H), 8.45 (d, J=8.0 Hz, 1H), 8.13 (s, 1H), 7.94 (d, J=3.1 Hz, 1H), 7.41 (d, J=8.0 Hz, 1H), 7.34 (d, J=12.4 Hz, 1H), 3.27 (d, J=7.8 Hz, 2H), 3.13-3.07 (m, 4H), 3.05-2.99 (m, 4H), 2.36 (s, 3H), 1.45 (t, J=7.6 Hz, 3H).

### Example 81: Synthesis of Compound 186

### Synthesis of Intermediate C84

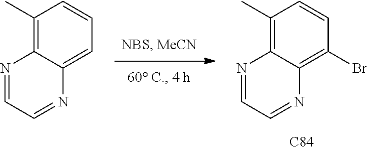

A mixture of 5-methylquinoxaline (36 g, 249.693 mmol, 1 equiv), NBS (88.88 g, 499.386 mmol, 2 equiv) and acetonitrile (700 mL) was deoxygenated with nitrogen for five minutes. The resulting mixture was stirred at 60° C. for 2 hours. After cooling to room temperature, the reaction mixture was filtered and the filtrate extracted with dichloromethane. The organic phase was dried over anhydrous MgSO₄, filtered, and the filtrate concentrated in vacuo to give a residue. The residue was purified by column chromatography on silica gel, eluted with petroleum ether: dichloromethane (1:2) to afford 5-bromo-8-methylquinoxaline (15.2 g, 27%) as a solid.

### Synthesis of Intermediate C85

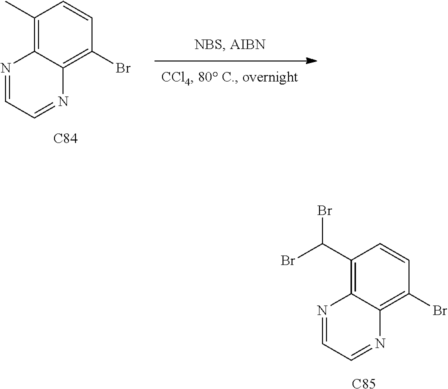

To a solution of 5-bromo-8-methylquinoxaline (5.1 g, 22.862 mmol, 1 equiv) in carbon tetrachloride (100 mL) was added NBS (8.14 g, 45.724 mmol, 2 equiv) and AIBN (1.88 g, 11.431 mmol, 0.5 equiv). The reaction mixture was stirred overnight at 100° C. under a nitrogen atmosphere, then concentrated under reduced pressure to give a residue. The residue was purified by silica gel column chromatography, eluted with petroleum ether: dichloromethane (1:2) to afford 5-bromo-8-(dibromomethyl) quinoxaline (8.2 g, 94%) as a solid. LCMS (ES, m/z): 381 [M+H]⁺.

### Synthesis of Intermediate C86

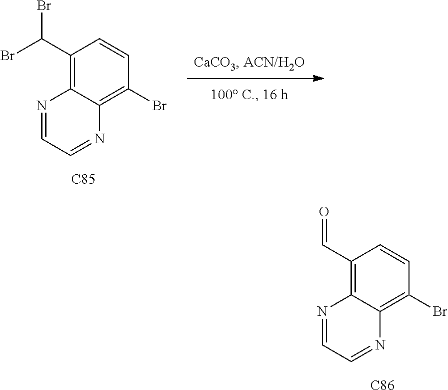 [SMILES: Brc1ccc(C(Br)Br)c2nccnc12 ; "C85" ; O=Cc1ccc(Br)c2nccnc12 ; "C86"]

To a mixture of 5-bromo-8-(dibromomethyl) quinoxaline (8.1 g, 21.267 mmol, 1 equiv) in acetonitrile (100 mL, 3843.493 mmol, 180.72 equiv) and water (50 mL, 2775.465 mmol, 130.50 equiv) was added CaCO₃ (649.21 g, 6486.435 mmol, 305 equiv). The reaction mixture was stirred for 16 h at 100° C. under a nitrogen atmosphere, then concentrated under reduced pressure. The resulting aqueous layer was extracted with DCM three times. The organic layers were combined and concentrated under vacuum to give a residue. The residue was purified by silica gel column chromatography, eluted with PE:EA (1:1) to afford 8-bromoquinoxaline-5-carbaldehyde (4.2 g, 83%) as an oil. LCMS (ES, m/z): 237 [M+H]⁺.

### Synthesis of Intermediate C87

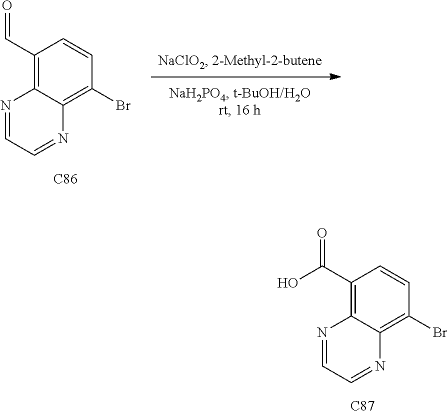 [SMILES: O=Cc1ccc(Br)c2nccnc12 ; "C86" ; O=C(O)c1ccc(Br)c2nccnc12 ; "C87"]

To a mixture of 8-bromoquinoxaline-5-carbaldehyde (4.1 g, 17.295 mmol, 1 equiv) in 2-methyl-2-propanol (76 mL, 1025.323 mmol, 59.28 equiv) and water (76 mL, 4218.707 mmol, 243.92 equiv) was added 3-methyl-1-butene (12.13 g, 172.950 mmol, 10 equiv), sodium chlorite (3.91 g, 43.238 mmol, 2.5 equiv) and NaH₂PO₄ (12.45 g, 103.770 mmol, 6 equiv). The reaction mixture was stirred for 16 h at 60° C. under a nitrogen atmosphere, then acidified to pH 6-7. A precipitate formed that was collected by filtration and washed with DCM three times to afford 8-bromoquinoxaline-5-carboxylic acid (2.6 g, 59%). LCMS (ES, m/z): 253 [M+H]⁺.

### Synthesis of Intermediate C88

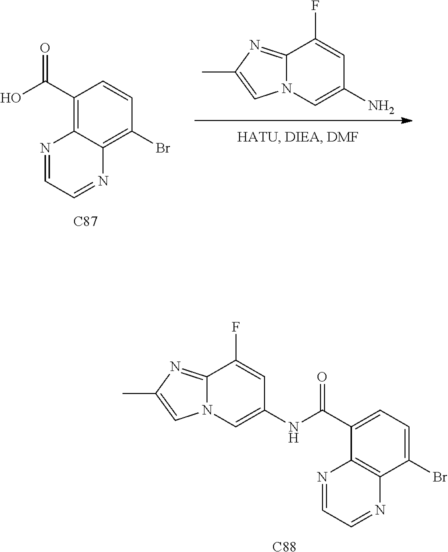 [SMILES: O=C(O)c1ccc(Br)c2nccnc12 ; Cc1cn2cc(N)cc(F)c2n1 ; "C87" ; Cc1cn2cc(NC(=O)c3ccc(Br)c4nccnc34)cc(F)c2n1 ; "C88"]

To a solution of 8-bromoquinoxaline-5-carboxylic acid (2.5 g, 9.879 mmol, 1 equiv) in dimethylformamide (35 mL) was added HATU (4.88 g, 12.843 mmol, 1.30 equiv) and DIEA (3.83 g, 29.637 mmol, 3.00 equiv). The reaction mixture was stirred for 10 min, then 8-fluoro-2-methylimidazo[1,2-a] pyridin-6-amine (1.79 g, 10.867 mmol, 1.10 equiv) was added. The resulting mixture was stirred for 4 h at room temperature. A precipitate formed that was collected by filtration and washed with ethyl acetate to afford 8-bromo-N-{8-fluoro-2-methylimidazo[1,2-a] pyridin-6-yl} quinoxaline-5-carboxamide (3.2 g, 81%) as a solid. LCMS (ES, m/z):400[M+H]⁺.

### Synthesis of Intermediate C89

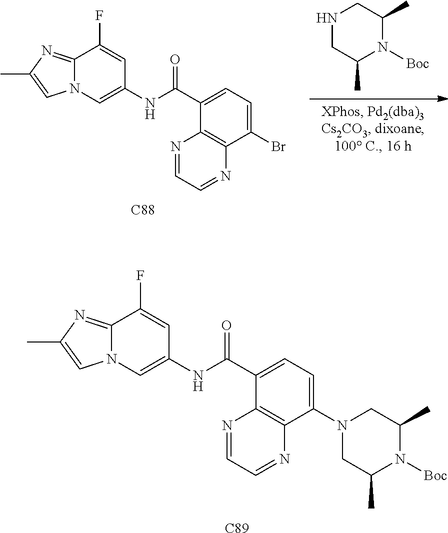

To a mixture of 8-bromo-N-{8-fluoro-2-methylimidazo[1,2-a] pyridin-6-yl}quinoxaline-5-carboxamide (115 mg, 0.287 mmol, 1 equiv) and tert-butyl 2,6-dimethylpiperazine-1-carboxylate (73.90 mg, 0.344 mmol, 1.20 equiv) in dioxane (10 mL) was added Cs₂CO₃ (187.83 mg, 0.574 mmol, 2.00 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl 4-[8-({8-fluoro-2-methylimidazo[1,2-a]pyridin-6-yl}carbamoyl)quinoxalin-5-yl]-2,6-dimethylpiperazine-1-carboxylate (53 mg, 35%) as an oil. LCMS (ES, m/z):534 [M+H]⁺.

### Synthesis of Compound 186

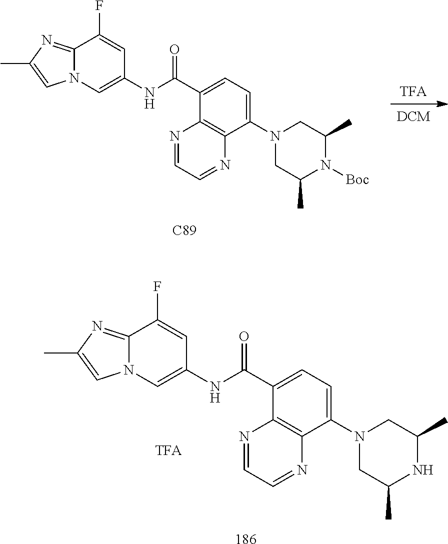

A solution of tert-butyl 4-[8-({8-fluoro-2-methylimidazo[1,2-a]-pyridin-6-yl} carbamoyl) quinoxalin-5-yl]-2,6-dimethylpiperazine-1-carboxylate (53 mg, 0.099 mmol, 1 equiv) in DCM was treated with TFA (4 mL). The reaction mixture was stirred for 1 h at room temperature. The resulting mixture was concentrated under reduced pressure to give a residue. The residue was purified by Prep-HPLC (Condition 5, Gradient 1, Gradient 2) to afford 8-(3,5-dimethylpiperazin-1-yl)-N-{8-fluoro-2-methylimidazo[1,2-a]pyridin-6-yl}quinoxaline-5-carboxamide (20 mg, 46%) as a solid. LCMS (ES, m/z):434 [M+H]⁺. ¹H NMR (300 MHz, DMSO-d₆) δ 12.55 (s, 1H), 9.50 (d, J=1.6 Hz, 1H), 9.27 (d, J=10.6 Hz, 1H), 9.17 (d, J=1.8 Hz, 1H), 9.09 (d, J=1.8 Hz, 1H), 8.64 (d, J=11.1 Hz, 1H), 8.55 (d, J=8.4 Hz, 1H), 8.15 (dd, J=2.6, 1.2 Hz, 1H), 7.88 (d, J=12.2 Hz, 1H), 7.49 (d, J=8.5 Hz, 1H), 4.31 (d, J=13.0 Hz, 2H), 3.64 (s, 2H), 3.12-2.97 (m, 2H), 2.44 (d, J=0.9 Hz, 3H), 1.31 (d, J=6.5 Hz, 6H).

### Example 82: Synthesis of Compound 187

### Synthesis of Intermediate C90

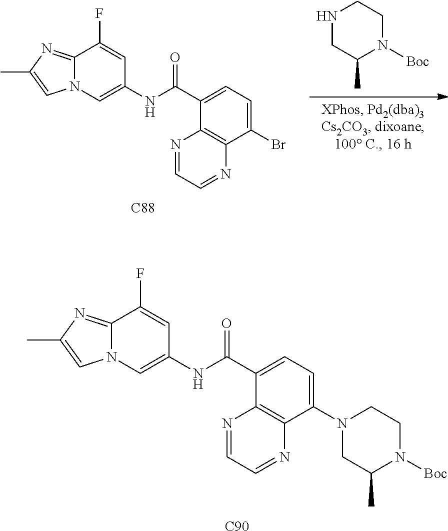

To a mixture of 8-bromo-N-{8-fluoro-2-methylimidazo[1,2-a] pyridin-6-yl} quinoxaline-5-carboxamide (115 mg, 0.287 mmol, 1 equiv) and tert-butyl 2-methylpiperazine-1-carboxylate (69.06 mg, 0.344 mmol, 1.2 equiv) in dioxane (5.75 mL) was added Cs₂CO₃ (187.83 mg, 0.574 mmol, 2 equiv), XPhos (13.70 mg, 0.029 mmol, 0.1 equiv) and Pd₂(dba)₃ (26.31 mg, 0.029 mmol, 0.1 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl 4-[8-({8-fluoro-2-methylimidazo[1,2-a]pyridin-6-yl}carbamoyl)quinoxalin-5-yl]-2-methylpiperazine-1-carboxylate (48 mg, 32%) as an oil. LCMS (ES, m/z): 520 [M+H]⁺.

### Synthesis of Compound 187

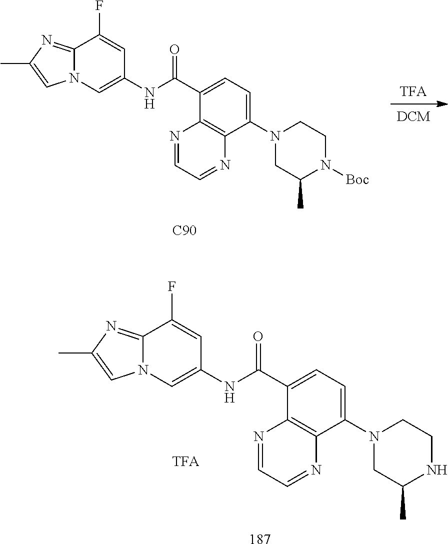

A solution of tert-butyl 4-[8-({8-fluoro-2-methylimidazo[1,2-a]pyridin-6-yl}carbamoyl)quinoxalin-5-yl]-2-methylpiperazine-1-carboxylate (48 mg, 0.092 mmol, 1 equiv) in DCM was treated with trifluoroacetaldehyde (4 mL). The reaction mixture was stirred for 1 h at room temperature, then concentrated under reduced pressure to give a residue. The residue was purified by Prep-HPLC (Condition 5, Gradient 3, Gradient 4) to afford N-{8-fluoro-2-methylimidazo[1,2-a] pyridin-6-yl}-8-(3-methylpiperazin-1-yl) quinoxaline-5-carboxamide (20 mg, 52%). LCMS (ES, m/z): 420 [M+H]⁺. ¹H NMR (300 MHz, DMSO-d₆) δ 12.56 (s, 1H), 9.52 (d, J=1.6 Hz, 1H), 9.23 (s, 1H), 9.17 (d, J=1.8 Hz, 1H), 9.09 (d, J=1.8 Hz, 1H), 8.89 (s, 1H), 8.55 (d, J=8.4 Hz, 1H), 8.17 (dd, J=2.7, 1.2 Hz, 1H), 7.96-7.86 (m, 1H), 7.48 (d, J=8.5 Hz, 1H), 4.27-4.16 (m, 4H), 3.35 (t, J=9.5 Hz, 2H), 3.17 (dd, J=13.1, 10.3 Hz, 1H), 2.45 (d, J=0.9 Hz, 3H), 1.33 (d, J=6.5 Hz, 3H).

### Example 83: Synthesis of Compound 188

### Synthesis of Intermediate C91

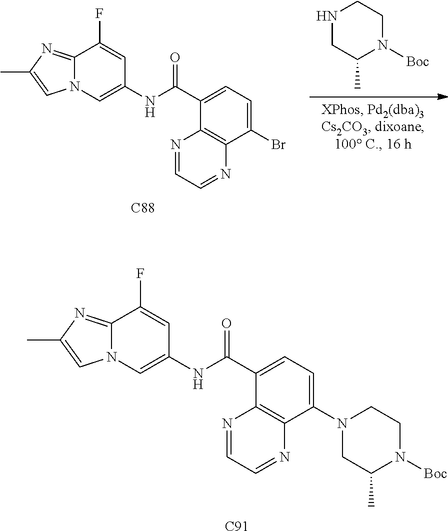

To a mixture of 8-bromo-N-{8-fluoro-2-methylimidazo[1,2-a] pyridin-6-yl} quinoxaline-5-carboxamide (115 mg, 0.287 mmol, 1 equiv) and tert-butyl 2-methylpiperazine-1-carboxylate (69.06 mg, 0.344 mmol, 1.2 equiv) in dioxane (10 mL) was added Cs₂CO₃ (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl 4-[8-({8-fluoro-2-methylimidazo[1,2-a]pyridin-6-yl}carbamoyl)quinoxalin-5-yl]-2-methylpiperazine-1-carboxylate (30 mg, 20%) as an oil. LCMS (ES, m/z): 520 [M+H]⁺.

### Synthesis of Compound 188

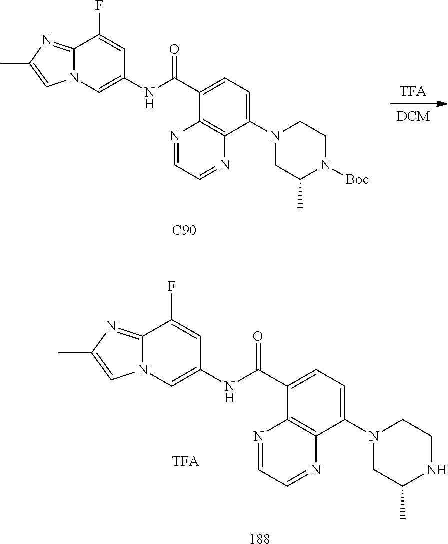

A solution of tert-butyl 4-[8-({8-fluoro-2-methylimidazo[1,2-a]pyridin-6-yl}carbamoyl)quinoxalin-5-yl]-2-methylpiperazine-1-carboxylate (30 mg, 0.058 mmol, 1 equiv) in DCM was treated with TFA (4 mL). The reaction mixture was stirred for 1 h at room temperature, then concentrated under reduced pressure to give a residue. The residue was purified by Prep-HPLC (Condition 5, Gradient 3, Gradient 4) to afford N-{8-fluoro-2-methylimidazo[1,2-a] pyridin-6-yl}-8-(3-methylpiperazin-1-yl)quinoxaline-5-carboxamide (17 mg, 70%) as a solid. LCMS (ES, m/z): 420 [M+H]⁺. ¹H NMR (300 MHz, DMSO-d₆) δ 12.58 (s, 1H), 9.53 (d, J=1.5 Hz, 1H), 9.29 (s, 1H), 9.16 (d, J=1.8 Hz, 1H), 9.08 (d, J=1.8 Hz, 1H), 8.94 (s, 1H), 8.54 (d, J=8.4 Hz, 1H), 8.22-8.14 (m, 1H), 8.00-7.89 (m, 1H), 7.47 (d, J=8.5 Hz, 1H), 4.26 (d, J=9.8 Hz, 1H), 4.16 (d, J=12.6 Hz, 1H), 3.60 (s, 1H), 3.49 (s, 1H), 3.34 (d, J=9.3 Hz, 2H), 3.17 (dd, J=13.1, 10.4 Hz, 1H), 2.49-2.42 (m, 3H), 1.33 (d, J=6.4 Hz, 3H).

### Example 84: Synthesis of Compound 189

### Synthesis of Intermediate C92

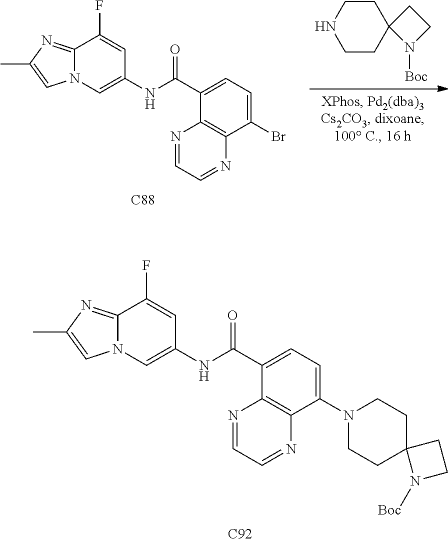 [SMILES: Cc1cn2cc(NC(=O)c3ccc(Br)c4nccnc34)cc(F)c2n1 ; CC(C)(C)OC(=O)N1CCC12CCNCC2 ; "XPhos, Pd2(dba)3Cs2CO3, dixoane,100° C., 16 h" ; "C88" ; Cc1cn2cc(NC(=O)c3ccc(N4CCC5(CC4)CCN5C(=O)OC(C)(C)C)c4nccnc34)cc(F)c2n1 ; "C92"]

To a mixture of 8-bromo-N-{8-fluoro-2-methylimidazo[1,2-a] pyridin-6-yl}quinoxaline-5-carboxamide (115 mg, 0.287 mmol, 1 equiv) and tert-butyl 1,7-diazaspiro[3.5]nonane-1-carboxylate (78.04 mg, 0.344 mmol, 1.2 equiv) in dioxane (10 mL) was added Cs₂CO₃ (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl 7-[8-({8-fluoro-2-methylimidazo[1,2-a]pyridin-6-yl}carbamoyl)quinoxalin-5-yl]-1,7-diazaspiro[3.5]nonane-1-carboxylate (58 mg, 37%) as an oil. LCMS (ES, m/z): 546 [M+H]⁺.

### Synthesis of Compound 189

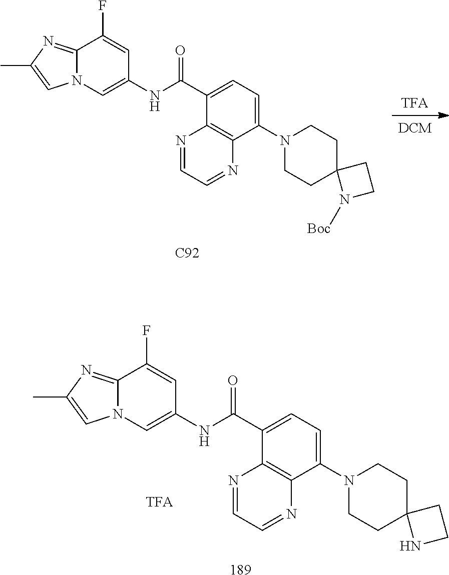

A solution of tert-butyl 7-[8-({8-fluoro-2-methylimidazo[1,2-a]pyridin-6-yl}carbamoyl)quinoxalin-5-yl]-1,7-diazaspiro[3.5]nonane-1-carboxylate (58 mg, 0.106 mmol, 1 equiv) in DCM was treated with TFA (4 mL). The reaction mixture was stirred for 1 h at room temperature, then concentrated under reduced pressure to give a residue. The residue was purified by Prep-HPLC (Condition 5, Gradient 5, Gradient 6) to afford 8-{1,7-diazaspiro[3.5] nonan-7-yl}-N-{8-fluoro-2-methylimidazo[1,2-a]pyridin-6-yl}quinoxaline-5-carboxamide (20 mg, 42%) as a solid. LCMS (ES, m/z): 446 [M+H]⁺. ¹H NMR (300 MHz, DMSO-d₆) δ 12.63 (s, 1H), 9.53 (d, J=1.5 Hz, 1H), 9.14 (d, J=1.8 Hz, 1H), 9.07 (d, J=1.8 Hz, 1H), 8.54 (d, J=8.5 Hz, 1H), 8.17 (d, J=2.6 Hz, 1H), 7.94 (d, J=12.1 Hz, 1H), 7.39 (d, J=8.6 Hz, 1H), 3.92 (q, J=7.4 Hz, 2H), 3.78-3.68 (m, 2H), 3.56-3.45 (m, 2H), 2.48-2.35 (m, 5H), 2.34-2.19 (m, 4H).

### Example 85: Synthesis of Compounds 190, 193, and 194

### Synthesis of Intermediate C93

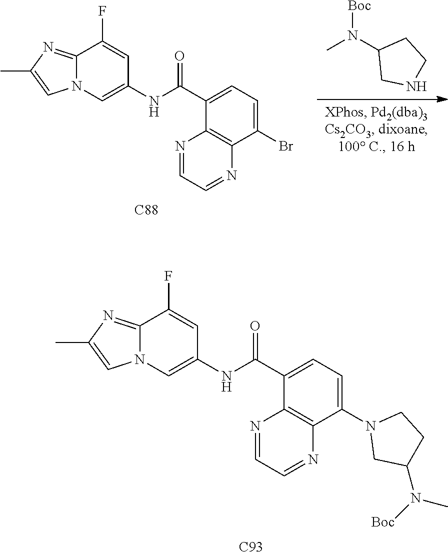

To a solution of 8-bromo-N-{8-fluoro-2-methylimidazo[1,2-a]pyridin-6-yl}quinoxaline-5-carboxamide (115 mg, 0.287 mmol, 1 equiv) and tert-butyl N-methyl-N-(pyrrolidin-3-yl)carbamate (69.06 mg, 0.344 mmol, 1.2 equiv) in dioxane (10 mL) was added Cs₂CO₃ (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl N-{1-[8-({8-fluoro-2-methylimidazo[1,2-a]pyridin-6-yl}carbamoyl)quinoxalin-5-yl]pyrrolidin-3-yl}-N-methylcarbamate (47 mg, 31%) as an oil. LCMS (ES, m/z): 520 [M+H]⁺.

### Synthesis of Compound 190

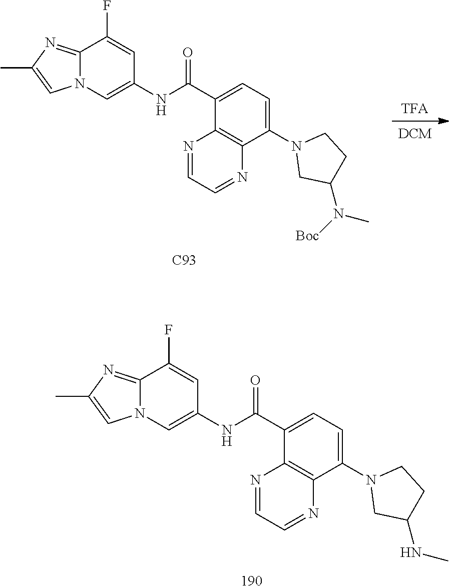

A solution of tert-butyl N-{1-[8-({8-fluoro-2-methylimidazo[1,2-a] pyridin-6-yl} carbamoyl) quinoxalin-5-yl]pyrrolidin-3-yl}-N-methylcarbamate (47 mg, 0.090 mmol, 1 equiv) in DCM was treated with TFA (4 mL). The reaction mixture was stirred for 1 h at room temperature, then concentrated under reduced pressure to give a residue. The residue was purified by Prep-HPLC (Condition 5, Gradient 3, Gradient 4) to afford N-{8-fluoro-2-methylimidazo[1,2-a]pyridin-6-yl}-8-[3-(methylamino)pyrrolidin-1-yl]quinoxaline-5-carboxamide (19 mg, 50%) as a solid. LCMS (ES, m/z): 420 [M+H]⁺. ¹H NMR (300 MHz, DMSO-d₆) δ 12.88 (s, 1H), 9.54 (d, J=1.5 Hz, 1H), 9.09 (d, J=1.8 Hz, 1H), 8.96 (d, J=1.8 Hz, 1H), 8.55 (d, J=9.0 Hz, 1H), 8.22-8.15 (m, 1H), 8.01 (d, J=12.1 Hz, 1H), 6.98 (d, J=9.0 Hz, 1H), 4.24 (qd, J=13.0, 5.4 Hz, 2H), 4.02 (dd, J=18.7, 7.5 Hz, 2H), 3.91-3.79 (m, 1H), 2.70 (s, 3H), 2.70 (d, J=9.7 Hz, 3H), 2.45-2.36 (m, 1H), 2.25 (dd, J=12.6, 6.4 Hz, 1H).

### Synthesis of Compounds 193 and 194

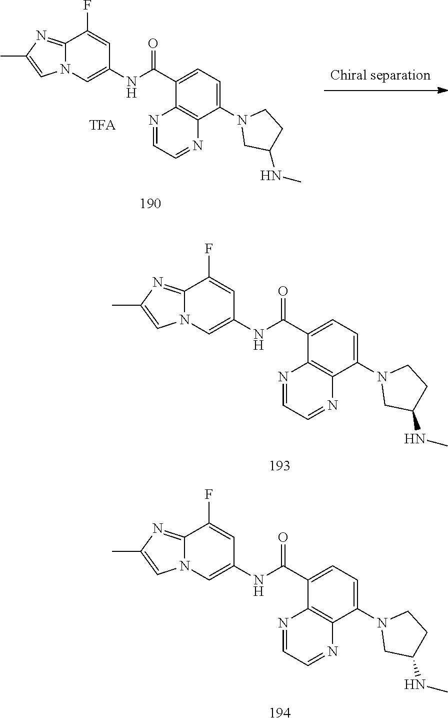

N-{8-fluoro-2-methylimidazo[1,2-a]pyridin-6-yl}-8-[3-(methylamino)pyrrolidin-1-yl]quinoxaline-5-carboxamide was purified by CHIRAL-HPLC (Condition 1) to afford Compound 193 (first peak, 5.3 mg) and Compound 194 (second peak, 2.4 mg) as solids.

### Example 86: Synthesis of Compound 191

### Synthesis of Intermediate C94

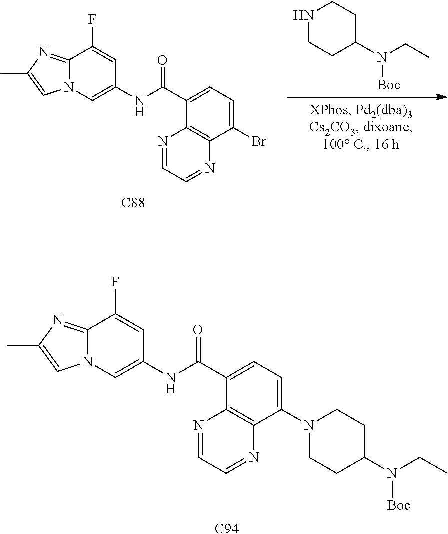 [SMILES: Cc1cn2cc(NC(=O)c3ccc(Br)c4nccnc34)cc(F)c2n1 ; CCN(C(=O)OC(C)(C)C)C1CCNCC1 ; "XPhos, Pd2(dba)3Cs2CO3, dixoane,100° C., 16 h" ; "C88" ; CCN(C(=O)OC(C)(C)C)C1CCN(c2ccc(C(=O)Nc3cc(F)c4nc(C)cn4c3)c3nccnc23)CC1 ; "C94"]

To a mixture of 8-bromo-N-{8-fluoro-2-methylimidazo[1,2-a]pyridin-6-yl}quinoxaline-5-carboxamide (115 mg, 0.287 mmol, 1 equiv) and tert-butyl N-ethyl-N-(piperidin-4-yl)carbamate (78.73 mg, 0.344 mmol, 1.2 equiv) in dioxane (10 mL) was added Cs₂CO₃ (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate concentrated under reduced pressure to give a residue. The residue was purified by silica gel column chromatography, eluted with PE:EA (1:2) to afford tert-butyl N-ethyl-N-{1-[8-({8-fluoro-2-methylimidazo[1,2-a]pyridin-6-yl}carbamoyl)quinoxalin-5-yl]piperidin-4-yl}carbamate (50 mg, 32%) as an oil. LCMS (ES, m/z): 548 [M+H]⁺.

### Synthesis of Compound 191

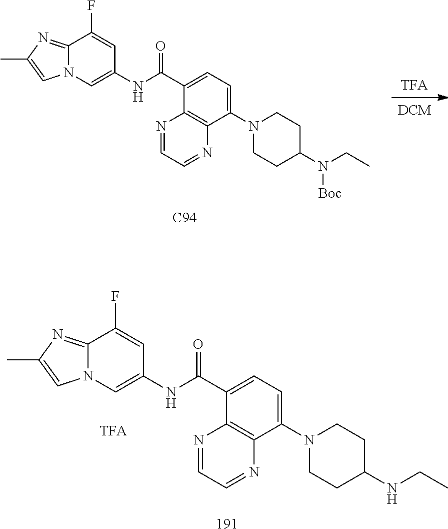

A solution of tert-butyl N-ethyl-N-{1-[8-({8-fluoro-2-methylimidazo[1,2-a] pyridin-6-yl}carbamoyl) quinoxalin-5-yl]piperidin-4-yl}carbamate (50 mg, 0.091 mmol, 1 equiv) in DCM was treated with TFA (4 mL). The reaction mixture was stirred for 1 h at room temperature, then concentrated under reduced pressure to give a residue. The residue was purified by Prep-HPLC (Condition 5, Gradient 3, Gradient 4) to afford 8-[4-(ethylamino) piperidin-1-yl]-N-{8-fluoro-2-methylimidazo[1,2-a] pyridin-6-yl}quinoxaline-5-carboxamide (15 mg, 37%) as a solid. LCMS (ES, m/z): 448 [M+H]⁺. ¹H NMR (300 MHz, DMSO-d₆) δ 12.70 (s, 1H), 9.57 (d, J=1.5 Hz, 1H), 9.10 (dd, J=19.6, 1.8 Hz, 2H), 8.70 (s, 2H), 8.52 (d, J=8.5 Hz, 1H), 8.21 (d, J=2.5 Hz, 1H), 8.12-7.97 (m, 1H), 7.38 (d, J=8.6 Hz, 1H), 4.25 (d, J=12.3 Hz, 2H), 3.37 (s, 1H), 3.23-2.92 (m, 4H), 2.63-2.51 (m, 3H), 2.18 (d, J=12.1 Hz, 2H), 1.83 (td, J=12.9, 9.1 Hz, 2H), 1.25 (t, J=7.1 Hz, 3H).

### Example 87: Synthesis of Compound 192

### Synthesis of Intermediate C95

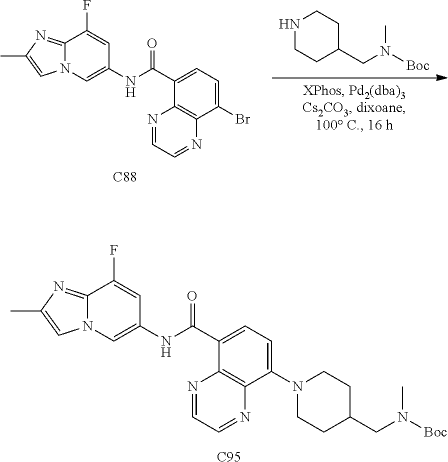 [SMILES: Cc1cn2cc(NC(=O)c3ccc(Br)c4nccnc34)cc(F)c2n1 ; CN(CC1CCNCC1)C(=O)OC(C)(C)C ; "XPhos, Pd2(dba)3Cs2CO3, dixoane,100° C., 16 h" ; "C88" ; Cc1cn2cc(NC(=O)c3ccc(N4CCC(CN(C)C(=O)OC(C)(C)C)CC4)c4nccnc34)cc(F)c2n1 ; "C95"]

To a mixture of 8-bromo-N-{8-fluoro-2-methylimidazo[1,2-a] pyridin-6-yl}quinoxaline-5-carboxamide (115 mg, 0.287 mmol, 1 equiv) and tert-butyl N-methyl-N-(piperidin-4-ylmethyl)carbamate (78.73 mg, 0.344 mmol, 1.2 equiv) in dioxane (10 mL) was added Cs₂CO₃ (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). The reaction mixture was stirred overnight at 100° C. under a nitrogen atmosphere, then filtrated, and the filtrate was concentrated under reduced pressure to give a residue. The residue was purified by silica gel column chromatography, eluted with PE:EA (1:2) to afford tert-butyl N-({1-[8-({8-fluoro-2-methylimidazo[1,2-a]pyridin-6-yl}carbamoyl)quinoxalin-5-yl]piperidin-4-yl}methyl)-N-methylcarbamate (40 mg, 25%) as an oil. LCMS (ES, m/z): 548[M+H]⁺.

### Synthesis of Compound 192

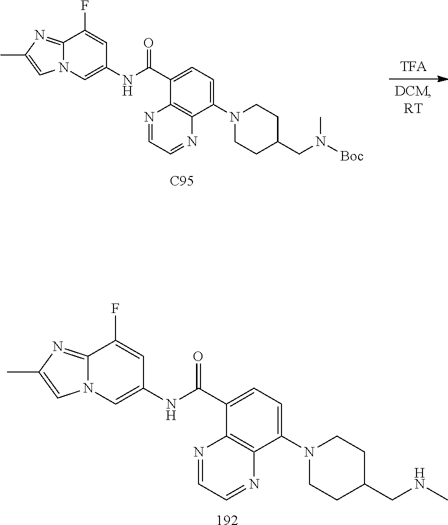

A solution of tert-butyl N-({1-[8-({8-fluoro-2-methylimidazo[1,2-a]pyridin-6-yl}carbamoyl)quinoxalin-5-yl]piperidin-4-yl}methyl)-N-methylcarbamate (40 mg, 0.073 mmol, 1 equiv) in DCM was treated with TFA (4 mL). The reaction mixture was stirred for 1 h at room temperature, then concentrated under reduced pressure to give a residue. The residue was purified by Prep-HPLC (Condition 5, Gradient 5, Gradient 6) to afford N-{8-fluoro-2-methylimidazo[1,2-a]pyridin-6-yl}-8-{4-[(methylamino)methyl]piperidin-1-yl}quinoxaline-5-carboxamide trifluoroacetate (6.6 mg, 20%) as an oil. LCMS (ES, m/z): 448[M+H]⁺. ¹H NMR (300 MHz, DMSO-d₆) δ 12.74 (s, 1H), 9.60 (d, J=1.5 Hz, 1H), 9.12 (d, J=1.8 Hz, 1H), 9.04 (d, J=1.8 Hz, 1H), 8.53 (d, J=8.5 Hz, 1H), 8.24 (s, 1H), 8.09 (dd, J=12.0, 1.6 Hz, 1H), 7.35 (d, J=8.6 Hz, 1H), 4.18 (d, J=12.1 Hz, 2H), 3.09-2.88 (m, 4H), 2.63 (t, J=5.3 Hz, 3H), 2.55 (s, 3H), 1.95 (d, J=14.2 Hz, 2H), 1.88 (s, 1H), 1.55 (t, J=11.6 Hz, 2H)

### Example 88: Synthesis of Compound 139

### Synthesis of Intermediate C96

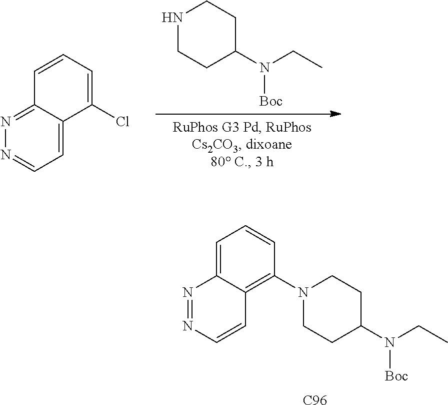 [SMILES: Clc1cccc2nnccc12 ; CCN(C(=O)OC(C)(C)C)C1CCNCC1 ; "RuPhos G3 Pd, RuPhosCs2CO3, dixoane80° C., 3 h" ; CCN(C(=O)OC(C)(C)C)C1CCN(c2cccc3nnccc23)CC1 ; "C96"]

To a stirred mixture of 5-chlorocinnoline (350.0 mg, 2.126 mmol, 1.0 equiv) and tert-butyl N-ethyl-N-(piperidin-4-yl)carbamate (582.6 mg, 2.551 mmol, 1.2 equiv) in dioxane (7 mL) was added Cs₂CO₃ (2.08 g, 6.378 mmol, 3.0 equiv), RuPhos (198.4 mg, 0.425 mmol, 0.2 equiv), and RuPhos Palladacycle Gen.3 (177.8 mg, 0.213 mmol, 0.1 equiv) at room temperature under nitrogen atmosphere. The resulting mixture was stirred for 3 h at 80° C. under nitrogen atmosphere, then concentrated under vacuum to give a residue. The residue was purified by silica gel column chromatography, eluted with PE/EA (1:1) to afford tert-butyl N-[1-(cinnolin-5-yl)piperidin-4-yl]-N-ethylcarbamate (500 mg, 66%) as a solid. LCMS (ES, m/z): 357 [M+H]⁺.

### Synthesis of Intermediate C97

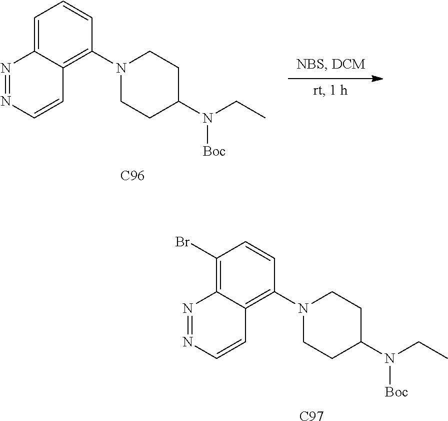

To a stirred mixture of tert-butyl N-[1-(cinnolin-5-yl) piperidin-4-yl]-N-ethylcarbamate (500.0 mg, 1.403 mmol, 1.0 equiv) in acetonitrile (10 mL) was added NBS (249.6 mg, 1.403 mmol, 1.0 equiv) at room temperature. The resulting mixture was stirred for 1 h at room temperature, then diluted with water (20 mL) and extracted with ethyl acetate (3×20 mL). The organic layers were combined, washed with water (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-[1-(8-bromocinnolin-5-yl) piperidin-4-yl]-N-ethylcarbamate (600 mg, 98%) as a solid. LCMS (ES, m/z): 435 [M+H]⁺.

### Synthesis of Intermediate C98

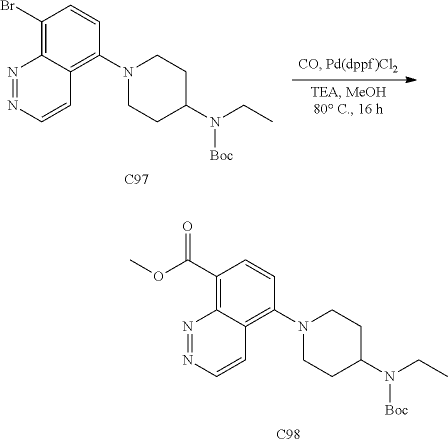 [SMILES: CCN(C(=O)OC(C)(C)C)C1CCN(c2ccc(Br)c3nnccc23)CC1 ; "CO, Pd(dppf)Cl2" ; "TEA, MeOH80° C., 16 h" ; "C97" ; CCN(C(=O)OC(C)(C)C)C1CCN(c2ccc(C(=O)OC)c3nnccc23)CC1 ; "C98"]

To a solution of tert-butyl N-[1-(8-bromocinnolin-5-yl) piperidin-4-yl]-N-ethylcarbamate (600 mg, 1.378 mmol, 1.0 equiv) in methanol (25 mL) was added TEA (418.3 mg, 4.134 mmol, 3.0 equiv) and Pd(dppf)Cl₂ (112.2 mg, 0.138 mmol, 0.1 equiv) in a pressure tank. The reaction mixture was purged with nitrogen for 2 min, then pressurized to 1 MPa with carbon monoxide and heated at 80° C. for 16 h. The reaction mixture was cooled to room temperature, filtered, and the filtrate concentrated under reduced pressure to give a residue. The residue was purified by silica gel column chromatography, eluted with EA to afford methyl 5-{4-[(tert-butoxycarbonyl) (ethyl)amino]piperidin-1-yl}cinnoline-8-carboxylate (500 mg, 88%) as a solid. LCMS (ES, m/z): 415 [M+H]⁺.

### Synthesis of Intermediate C99

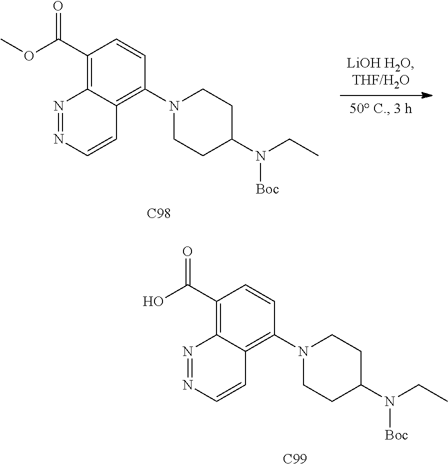 [SMILES: CCN(C(=O)OC(C)(C)C)C1CCN(c2ccc(C(=O)OC)c3nnccc23)CC1 ; "50° C., 3 h" ; "LiOH H2O,THF/H2O" ; "C98" ; CCN(C(=O)OC(C)(C)C)C1CCN(c2ccc(C(=O)O)c3nnccc23)CC1 ; "C99"]

Methyl 5-{4-[(tert-butoxycarbonyl)(ethyl)amino]piperidin-1-yl}cinnoline-8-carboxylate (280.0 mg, 0.676 mmol, 1.0 equiv), lithiumol (161.7 mg, 6.760 mmol, 10.0 equiv), tetrahydrofuran (3 mL), and water (3 mL) were combined at room temperature. The resulting mixture was stirred for 3 h at 50° C. The resulting mixture was diluted with deionized water (20 mL), acidified to pH 6 with HCl (2 N), and extracted with ethyl acetate (2×20 mL). The organic layers were combined, washed with brine (1×20 mL), dried over anhydrous Na₂SO₄, and filtered. After filtration, the filtrate was concentrated under reduced pressure to afford 5-{4-[(tert-butoxycarbonyl)(ethyl)amino]piperidin-1-yl}cinnoline-8-carboxylic acid (200 mg, 70%) as a solid. LCMS (ES, m/z): 401 [M+H]⁺.

### Synthesis of Intermediate C100

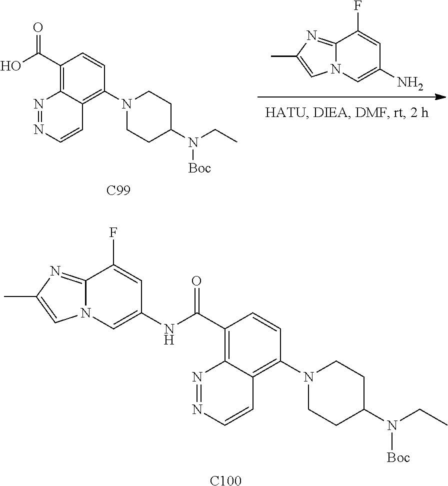 [SMILES: CCN(C(=O)OC(C)(C)C)C1CCN(c2ccc(C(=O)O)c3nnccc23)CC1 ; Cc1cn2cc(N)cc(F)c2n1 ; "C99" ; CCN(C(=O)OC(C)(C)C)C1CCN(c2ccc(C(=O)Nc3cc(F)c4nc(C)cn4c3)c3nnccc23)CC1 ; "C100"]

5-{4-[(tert-butoxycarbonyl)(ethyl)amino]piperidin-1-yl}cinnoline-8-carboxylic acid (100.0 mg, 0.250 mmol, 1.0 equiv), dimethylformamide (4 mL), 2,8-dimethylimidazo[1,2-a]pyrazin-6-amine (48.6 mg, 0.300 mmol, 1.2 equiv), DIEA (96.8 mg, 0.750 mmol, 3.0 equiv), and HATU (189.8 mg, 0.500 mmol, 2.0 equiv) were combined at room temperature. The resulting mixture was stirred for 2 h at room temperature, then extracted with ethyl acetate (2×20 mL). The organic layers were combined, washed with brine (1×20 mL), dried over anhydrous Na₂SO₄, and filtered. After filtration, the filtrate was concentrated under reduced pressure to give a residue. The residue was purified by silica gel column chromatography, eluted with CHCl₂/MeOH (40:1) to afford tert-butyl N-ethyl-N-{1-[8-({8-fluoro-2-methylimidazo[1,2-a]pyridin-6-yl}carbamoyl)cinnolin-5-yl]piperidin-4-yl}carbamate (120 mg, 79%) as a solid. LCMS (ES, m/z): 548 [M+H]⁺.

### Synthesis of Compound 139

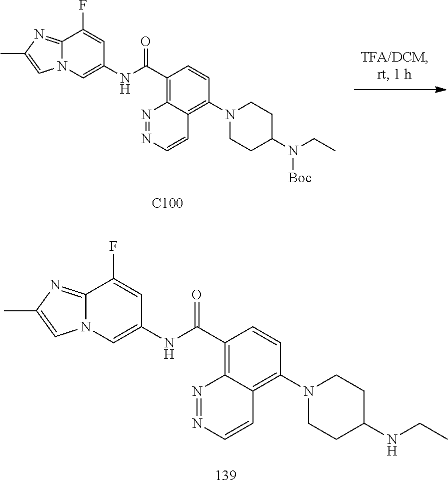

Tert-butyl N-ethyl-N-{1-[8-({8-fluoro-2-methylimidazo[1,2-a]pyridin-6-yl}carbamoyl)cinnolin-5-yl]piperidin-4-yl}carbamate(100.0 mg, 0.230 mmol, 1.0 equiv), DCM (2 mL), and TFA (0.5 mL) were stirred for 1 h at room temperature. The resulting mixture was concentrated under reduced pressure to give a residue. The residue was purified by Prep-HPLC with the following conditions (Condition 10, Gradient 1) to afford 5-[4-(ethylamino)piperidin-1-yl]-N-{8-fluoro-2-methylimidazo[1,2-a]pyridin-6-yl}cinnoline-8-carboxamide (29.3 mg, 36%) as a solid. LCMS (ES, m/z): 448 [M+H]⁺. ¹H NMR (300 MHz, DMSO-d₆) δ 12.16 (s, 1H), 9.50 (d, J=5.9 Hz, 1H), 9.22 (d, J=1.7 Hz, 1H), 8.48 (d, J=8.0 Hz, 1H), 8.30 (d, J=6.0 Hz, 1H), 7.98-7.90 (m, 1H), 7.44 (d, J=8.1 Hz, 1H), 7.31 (dd, J=12.4, 1.7 Hz, 1H), 3.46 (d, J=11.9 Hz, 2H), 2.93 (t, J=11.3 Hz, 2H), 2.71-2.61 (m, 3H), 2.36 (d, J=0.9 Hz, 3H), 2.02 (d, J=12.6 Hz, 2H), 1.62 (q, J=9.9 Hz, 2H), 1.06 (t, J=7.1 Hz, 3H).

### Example 89: Synthesis of Compound 197

### Synthesis of Intermediate C101

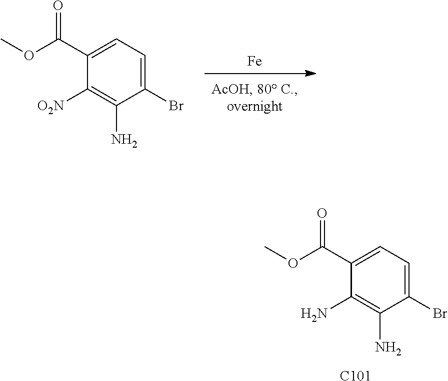

To a stirred solution of methyl 3-amino-4-bromo-2-nitrobenzoate (10 g, 36.356 mmol, 1 equiv) in acetic acid (200 mL) was added iron (10.15 g, 181.780 mmol, 5 equiv) in portions at room temperature. The resulting mixture was stirred overnight at 80° C., then cooled to room temperature. The resulting mixture was filtered, the filter cake washed with CH₂Cl₂ (3×50 mL), and the filtrate was concentrated under reduced pressure to afford methyl 2,3-diamino-4-bromobenzoate (14.1 g) as an oil. LCMS (ES, m/z): 245 [M+H]⁺.

### Synthesis of Intermediate C102

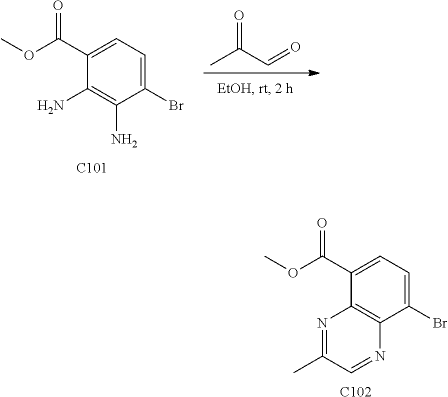

To a stirred solution of methyl 2,3-diamino-4-bromobenzoate (4 g, 16.3 mmol, 1 equiv) in ethanol (40 mL) was added 2-oxopropanal (1.41 g, 19.6 mmol, 1.2 equiv) dropwise at room temperature. The resulting mixture was stirred for 2 h at room temperature, then concentrated under reduced pressure to give a residue. The residue was purified by reverse phase flash with the following conditions (Condition 11, Gradient 1) to afford methyl 8-bromo-3-methylquinoxaline-5-carboxylate (850 mg, 19%) as a solid. LCMS (ES, m/z): 281 [M+H]⁺.

### Synthesis of Intermediate C103

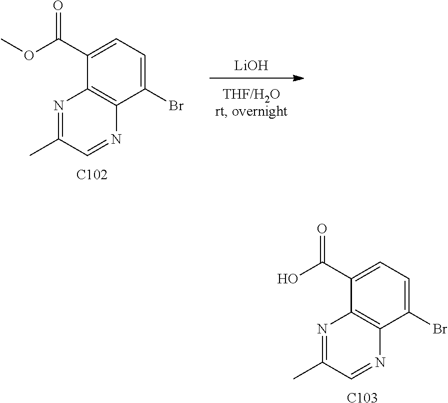 [SMILES: COC(=O)c1ccc(Br)c2ncc(C)nc12 ; "LiOH" ; "C102" ; Cc1cnc2c(Br)ccc(C(=O)O)c2n1 ; "C103"]

To a stirred solution of methyl 8-bromo-3-methylquinoxaline-5-carboxylate (210 mg, 0.747 mmol, 1 equiv) in THE (3 mL) and H₂O (1 mL) was added lithiumol (35.78 mg, 1.494 mmol, 2 equiv) at room temperature. The resulting mixture was stirred overnight at room temperature, then concentrated under vacuum, diluted with H₂O (5 mL), and acidified to pH 3 with citric acid. A precipitate formed that was collected by filtration and dried under infrared light to afford 8-bromo-3-methylquinoxaline-5-carboxylic acid (158 mg, 79%) as a solid. LCMS (ES, m/z): 267 [M+H]⁺.

### Synthesis of Intermediate C104

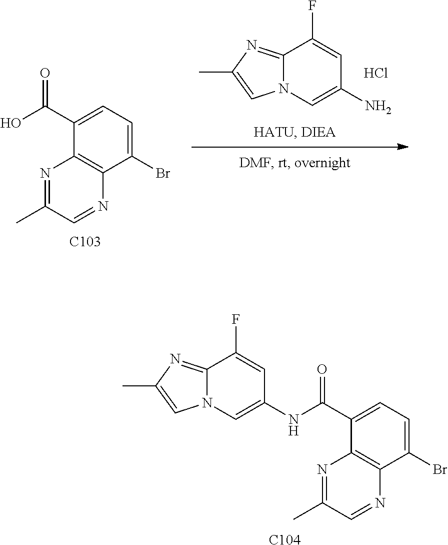

To a stirred mixture of 8-bromo-3-methylquinoxaline-5-carboxylic acid (158 mg, 0.592 mmol, 1 equiv) and HATU (269.93 mg, 0.710 mmol, 1.2 equiv) in DMF (3 mL) was added DIEA (267.61 mg, 2.072 mmol, 3.5 equiv) and 8-fluoro-2-methylimidazo[1,2-a]pyridin-6-amine hydrochloride (143.14 mg, 0.710 mmol, 1.2 equiv) at room temperature. The resulting mixture was stirred overnight at room temperature, then filtered. The filter cake was washed with diethyl ether (2×5 mL) and dried under infrared light to afford 8-bromo-N-{8-fluoro-2-methylimidazo[1,2-a]pyridin-6-yl}-3-methylquinoxaline-5-carboxamide (120 mg, 49%) as a solid. LCMS (ES, m/z): 414 [M+H]⁺.

### Synthesis of Intermediate C105

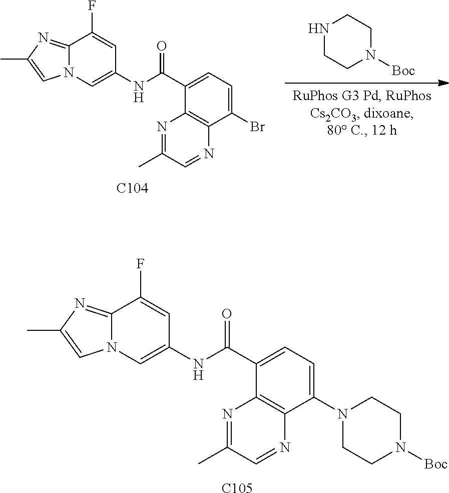 [SMILES: Cc1cnc2c(Br)ccc(C(=O)Nc3cc(F)c4nc(C)cn4c3)c2n1 ; CC(C)(C)OC(=O)N1CCNCC1 ; "RuPhos G3 Pd, RuPhosCs2CO3, dixoane,80° C., 12 h" ; "C104" ; Cc1cnc2c(N3CCN(C(=O)OC(C)(C)C)CC3)ccc(C(=O)Nc3cc(F)c4nc(C)cn4c3)c2n1 ; "C105"]

To a stirred mixture of 8-bromo-N-{8-fluoro-2-methylimidazo[1,2-a]pyridin-6-yl}-3-methylquinoxaline-5-carboxamide (85 mg, 0.205 mmol, 1 equiv) and tert-butyl piperazine-1-carboxylate (45.86 mg, 0.246 mmol, 1.2 equiv) in dioxane (1.5 mL) was added Cs₂CO₃ (200.57 mg, 0.615 mmol, 3 equiv), RuPhos (19.15 mg, 0.041 mmol, 0.2 equiv), and RuPhos Palladacycle Gen.3 (17.16 mg, 0.021 mmol, 0.1 equiv) at room temperature under nitrogen atmosphere. The reaction mixture was stirred for 12 h at 80° C. under nitrogen atmosphere. The resulting mixture was concentrated under reduced pressure to give a residue. The residue was purified by silica gel column chromatography, eluted with CH₂Cl₂/MeOH (10:1) to afford tert-butyl 4-[8-({8-fluoro-2-methylimidazo[1,2-a]pyridin-6-yl}carbamoyl)-2-methylquinoxalin-5-yl]piperazine-1-carboxylate (80 mg, 75%) as a solid. LCMS (ES, m/z): 520 [M+H]⁺.

### Synthesis of Compound 197

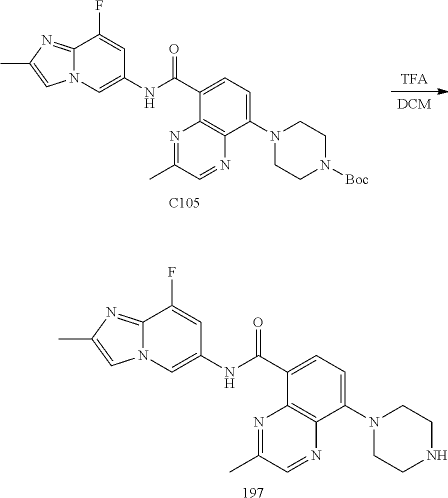

To a stirred solution of tert-butyl 4-[8-({8-fluoro-2-methylimidazo[1,2-a]pyridin-6-yl}carbamoyl)-2-methylquinoxalin-5-yl]piperazine-1-carboxylate (80 mg, 0.154 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC with the following conditions (Condition 12, Gradient 1) to afford N-{8-fluoro-2-methylimidazo[1,2-a]pyridin-6-yl}-3-methyl-8-(piperazin-1-yl)quinoxaline-5-carboxamide; trifluoroacetic acid (10.6 mg, 13%) as a solid. LCMS (ES, m/z): 420 [M+H]⁺. 1H NMR (400 MHz, DMSO-d₆) δ 12.67 (s, 1H), 9.44 (s, 1H), 8.98 (d, J=11.6 Hz, 3H), 8.55 (d, J=8.4 Hz, 1H), 8.12 (s, 1H), 7.67 (s, 1H), 7.40 (d, J=8.6 Hz, 1H), 3.77 (t, J=5.2 Hz, 4H), 3.39 (s, 4H), 2.92 (s, 3H), 2.44 (s, 3H).

### Example 90: Synthesis of Compound 205

### Synthesis of Intermediate C106

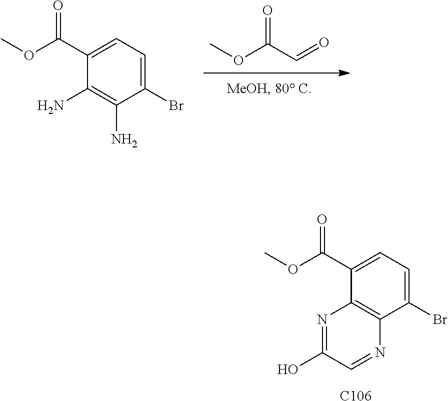

To a stirred mixture of methyl 2,3-diamino-4-bromobenzoate (10 g, 40.804 mmol, 1 equiv) and TEA (4.95 g, 48.965 mmol, 1.2 equiv) in ethanol (400 mL) was added methyl 2-oxoacetate (4.31 g, 48.965 mmol, 1.2 equiv) dropwise at room temperature. The resulting mixture was stirred for 2 h at 80° C., then concentrated under reduced pressure to give a residue. The residue was purified by prep-HPLC (Condition 11, Gradient 2) to afford methyl 8-bromo-3-hydroxyquinoxaline-5-carboxylate (1.5 g, 13%) as a solid. LCMS (ES, m/z): 283 [M+H]⁺.

### Synthesis of Intermediate C107

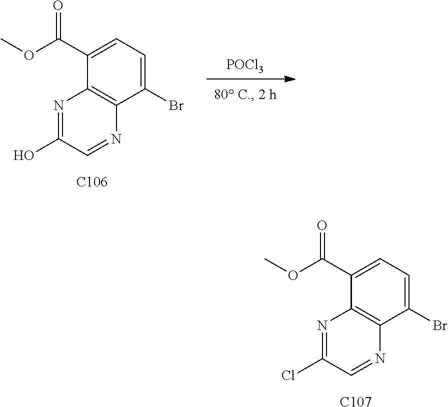 [SMILES: COC(=O)c1ccc(Br)c2ncc(O)nc12 ; "POCl3" ; "C106" ; COC(=O)c1ccc(Br)c2ncc(Cl)nc12 ; "C107"]

A solution of methyl 8-bromo-3-hydroxyquinoxaline-5-carboxylate (1.5 g, 5.299 mmol, 1 equiv) in phosphorus oxychloride (15 mL) was stirred for 2 h at 80° C. The reaction mixture was poured into water/ice (20 mL) and extracted with CH₂Cl₂ (3×30 mL). The organic layers were combined, washed with brine (1×20 mL), dried over anhydrous Na₂SO₄, and filtered. After filtration, the filtrate was concentrated under reduced pressure to afford methyl 8-bromo-3-chloroquinoxaline-5-carboxylate (1.3 g, 81%) as a solid. LCMS (ES, m/z): 301 [M+H]⁺.

### Synthesis of Intermediate C108

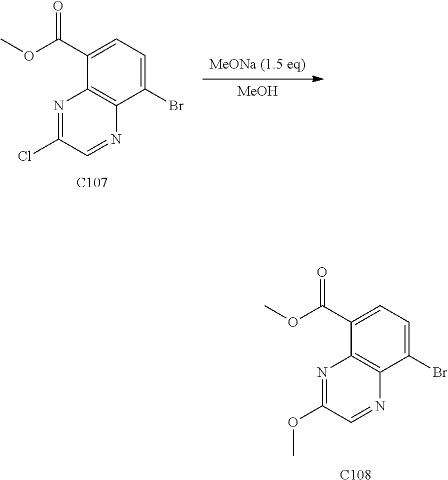 [SMILES: "MeOH" ; COC(=O)c1ccc(Br)c2ncc(Cl)nc12 ; "MeONa (1.5 eq)" ; "C107" ; COC(=O)c1ccc(Br)c2ncc(OC)nc12 ; "C108"]

To a stirred solution of methyl 8-bromo-3-chloroquinoxaline-5-carboxylate (1.3 g, 4.311 mmol, 1 equiv) in methanol (13 mL) was added MeONa (0.35 g, 6.466 mmol, 1.5 equiv) dropwise at room temperature. The resulting mixture was stirred for 2 h at room temperature, then concentrated under vacuum to give a residue. The residue was purified by silica gel column chromatography, eluted with PE/EA (4:1) to afford methyl 8-bromo-3-methoxyquinoxaline-5-carboxylate (600 mg, 47%) as a solid. LCMS (ES, m/z): 297 [M+H]⁺.

### Synthesis of Intermediate C109

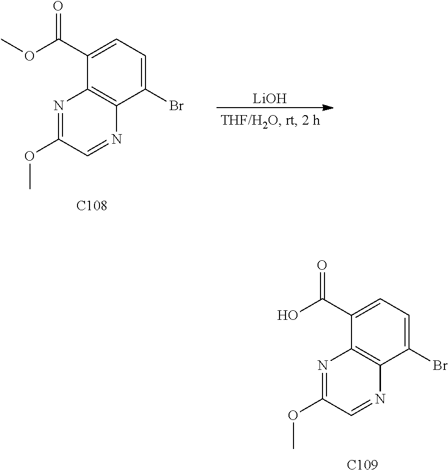 [SMILES: COC(=O)c1ccc(Br)c2ncc(OC)nc12 ; "LiOH" ; "C108" ; COc1cnc2c(Br)ccc(C(=O)O)c2n1 ; "C109"]

To a stirred solution of methyl 8-bromo-3-methoxyquinoxaline-5-carboxylate (500 mg, 1.683 mmol, 1 equiv) in THE (7.5 mL) and water (1 mL) was added lithiumol (80.61 mg, 3.366 mmol, 2 equiv) at room temperature. The resulting mixture was stirred overnight at room temperature, then concentrated under vacuum, diluted with water (10 mL), and acidified to pH 3 with citric acid. A precipitate formed that was collected by filtration and dried under infrared light to afford 8-bromo-3-methoxyquinoxaline-5-carboxylic acid (450 mg, 94%) as a solid. LCMS (ES, m/z): 283 [M+H]⁺.

### Synthesis of Intermediate C110

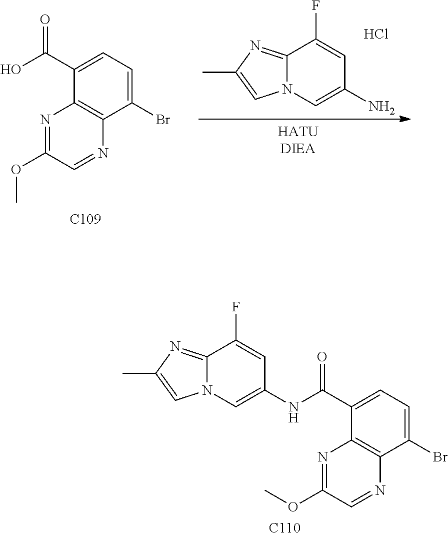 [SMILES: COc1cnc2c(Br)ccc(C(=O)O)c2n1 ; Cc1cn2cc(N)cc(F)c2n1 ; Cl ; "C109" ; COc1cnc2c(Br)ccc(C(=O)Nc3cc(F)c4nc(C)cn4c3)c2n1 ; "C110"]

To a stirred mixture of 8-bromo-3-methoxyquinoxaline-5-carboxylic acid (450 mg, 1.590 mmol, 1 equiv) and HATU (725.33 mg, 1.908 mmol, 1.2 equiv) in DMF (3 mL) was added DIEA (616.37 mg, 4.770 mmol, 3 equiv) and 8-fluoro-2-methylimidazo[1,2-a]pyridin-6-amine hydrochloride (384.63 mg, 1.908 mmol, 1.2 equiv) at room temperature. The resulting mixture was stirred overnight at room temperature. The reaction was filtered, the filter cake was washed with diethyl ether (2×5 mL), and the solid dried under reduced pressure to afford N-{8-fluoro-2-methylimidazo[1,2-a]pyridin-6-yl}-2-(6-methoxypyrazin-2-yl)prop-2-enamide (320 mg, 62%) as a solid. LCMS (ES, m/z): 430 [M+H]⁺.

### Synthesis of Intermediate C111

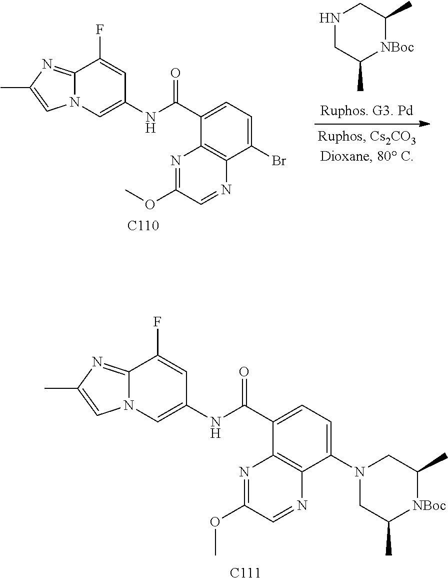

To a stirred mixture of 8-bromo-N-{8-fluoro-2-methylimidazo[1,2-a]pyridin-6-yl}-3-methoxyquinoxaline-5-carboxamide (100 mg, 0.232 mmol, 1 equiv) and tert-butyl (2R,6S)-2,6-dimethylpiperazine-1-carboxylate (59.77 mg, 0.278 mmol, 1.2 equiv) in dioxane (2 mL) was added Cs₂CO₃ (227.19 mg, 0.696 mmol, 3 equiv), Ruphos (21.69 mg, 0.046 mmol, 0.2 equiv), and RuPhos Palladacycle Gen.3 (19.44 mg, 0.023 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl (2R,6S)-4-[8-({8-fluoro-2-methylimidazo[1,2-a]pyridin-6-yl}carbamoyl)-2-methoxyquinoxalin-5-yl]-2,6-dimethylpiperazine-1-carboxylate (80 mg, 61%) as a solid. LCMS (ES, m/z): 564 [M+H]⁺.

### Synthesis of Compound 205

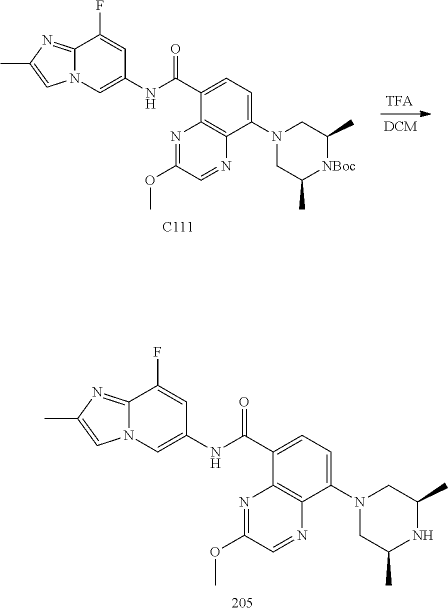

To a stirred solution of tert-butyl (2R,6S)-4-[8-({8-fluoro-2-methylimidazo[1,2-a]pyridin-6-yl}carbamoyl)-2-methoxyquinoxalin-5-yl]-2,6-dimethylpiperazine-1-carboxylate (80 mg, 0.142 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 12, Gradient 1) to afford 8-[(3R,5S)-3, 5-dimethylpiperazin-1-yl]-N-{8-fluoro-2-methylimidazo[1,2-a]pyridin-6-yl}-3-methoxyquinoxaline-5-carboxamide; trifluoroacetic acid (18.4 mg, 22%) as a solid. LCMS (ES, m/z): 464 [M+H]⁺. 1H NMR (400 MHz, DMSO-d₆) δ 11.50 (s, 1H), 9.33 (s, 1H), 9.12 (d, J=10.4 Hz, 1H), 8.70 (s, 1H), 8.52 (d, J=10.8 Hz, 1H), 8.41 (d, J=8.5 Hz, 1H), 8.05 (s, 1H), 7.45 (d, J=11.9 Hz, 1H), 7.32 (d, J=8.7 Hz, 1H), 4.23 (s, 5H), 2.99 (t, J=12.2 Hz, 2H), 2.40 (s, 3H), 1.29 (d, J=6.5 Hz, 6H).

### Example 91: Synthesis of Compound 201

### Synthesis of Intermediate C112

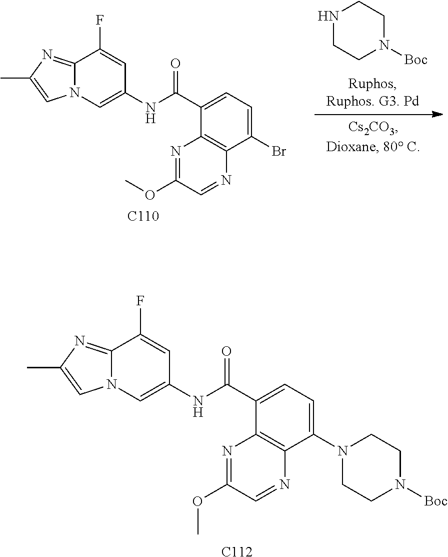 [SMILES: COc1cnc2c(Br)ccc(C(=O)Nc3cc(F)c4nc(C)cn4c3)c2n1 ; CC(C)(C)OC(=O)N1CCNCC1 ; "C110" ; COc1cnc2c(N3CCN(C(=O)OC(C)(C)C)CC3)ccc(C(=O)Nc3cc(F)c4nc(C)cn4c3)c2n1 ; "C112"]

To a stirred mixture of 8-bromo-N-{8-fluoro-2-methylimidazo[1,2-a]pyridin-6-yl}-3-methoxyquinoxaline-5-carboxamide (100 mg, 0.232 mmol, 1 equiv) and tert-butyl piperazine-1-carboxylate (51.95 mg, 0.278 mmol, 1.2 equiv) in dioxane (2 mL) was added Cs₂CO₃ (227.19 mg, 0.696 mmol, 3 equiv), Ruphos (21.69 mg, 0.046 mmol, 0.2 equiv), and RuPhos Palladacycle Gen.3 (19.44 mg, 0.023 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl 4-[8-({8-fluoro-2-methylimidazo[1,2-a]pyridin-6-yl}carbamoyl)-2-methoxyquinoxalin-5-yl]piperazine-1-carboxylate (60 mg, 48%) as a solid. LCMS (ES, m/z): 504 [M+H]⁺.

### Synthesis of Compound 201

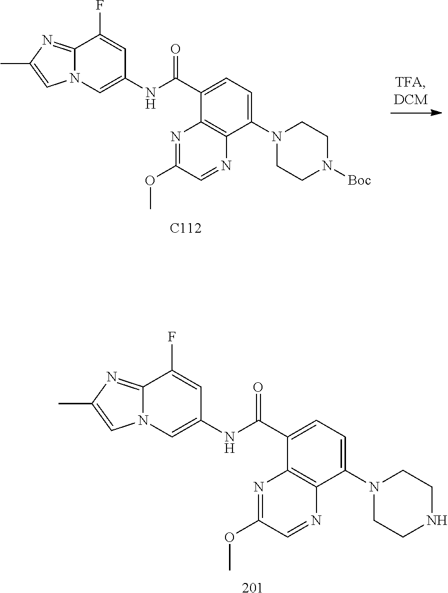

To a stirred solution of tert-butyl 4-[8-({8-fluoro-2-methylimidazo[1,2-a]pyridin-6-yl}carbamoyl)-2-methoxyquinoxalin-5-yl]piperazine-1-carboxylate (60 mg, 0.112 mmol, 1 equiv) in DCM (1 mL) was added TFA (1 mL, 13.463 mmol, 120.18 equiv) at room temperature. The resulting mixture was stirred for 2 h at room temperature, then concentrated under reduced pressure to give a residue. The residue was purified by Prep-HPLC (Condition 12, Gradient 1) to afford N-{8-fluoro-2-methylimidazo[1,2-a]pyridin-6-yl}-3-methoxy-8-(piperazin-1-yl)quinoxaline-5-carboxamide; bis(trifluoroacetic acid) (21.3 mg, 29%) as a solid. LCMS (ES, m/z): 436 [M+H]⁺. 1H NMR (400 MHz, Methanol-d₄) δ 9.40 (s, 1H), 8.66-8.59 (m, 2H), 8.03 (s, 1H), 7.71 (d, J=11.4 Hz, 1H), 7.31 (d, J=8.6 Hz, 1H), 4.34 (s, 3H), 3.85-3.78 (m, 4H), 3.57-3.50 (m, 4H), 2.56 (d, J=1.0 Hz, 3H).

### Example 92: Synthesis of Compound 202

### Synthesis of Intermediate C113

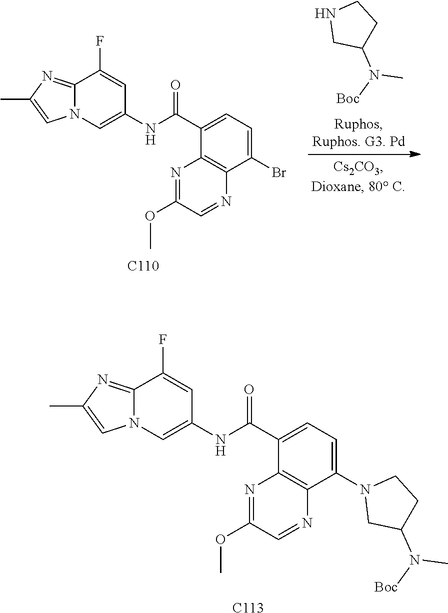

To a stirred mixture of 8-bromo-N-{8-fluoro-2-methylimidazo[1,2-a]pyridin-6-yl}-3-methoxyquinoxaline-5-carboxamide (50 mg, 0.116 mmol, 1 equiv) and tert-butyl N-methyl-N-(pyrrolidin-3-yl)carbamate (27.93 mg, 0.139 mmol, 1.2 equiv) in dioxane (1 mL) was added Cs₂CO₃ (113.60 mg, 0.348 mmol, 3 equiv), Ruphos (10.85 mg, 0.023 mmol, 0.2 equiv), and RuPhos Palladacycle Gen.3 (19.44 mg, 0.023 mmol, 0.2 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl N-{1-[8-({8-fluoro-2-methylimidazo[1,2-a]pyridin-6-yl}carbamoyl)-2-methoxyquinoxalin-5-yl]pyrrolidin-3-yl}-N-methylcarbamate (50 mg, 78%) as a solid. LCMS (ES, m/z): 550 [M+H]⁺.

### Synthesis of Compound 202

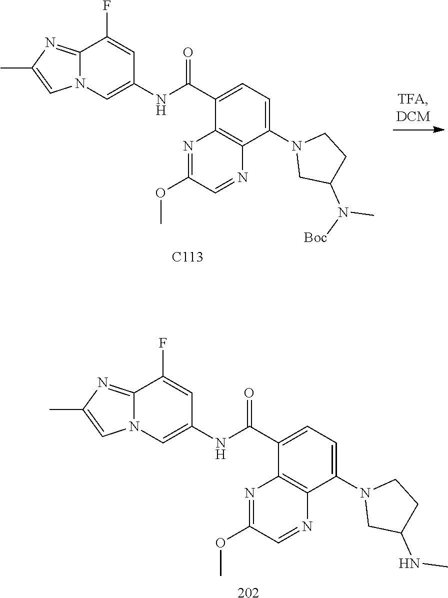

To a stirred solution of tert-butyl N-{1-[8-({8-fluoro-2-methylimidazo[1,2-a]pyridin-6-yl}carbamoyl)-2-methoxyquinoxalin-5-yl]pyrrolidin-3-yl}-N-methylcarbamate (50 mg, 0.091 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 12, Gradient 1) to afford N-{8-fluoro-2-methylimidazo[1,2-a]pyridin-6-yl}-3-methoxy-8-[3-(methylamino)pyrrolidin-1-yl]quinoxaline-5-carboxamide; bis(trifluoroacetic acid) (10.4 mg, 17%) as a solid. LCMS (ES, m/z): 450 [M+H]⁺. 1H NMR (400 MHz, Methanol-d₄) δ 9.30 (d, J=7.9 Hz, 1H), 8.54 (dd, J=8.9, 3.3 Hz, 1H), 8.45 (d, J=6.1 Hz, 1H), 7.99 (s, 1H), 7.55 (d, J=12.2 Hz, 1H), 6.88 (d, J=9.0 Hz, 1H), 4.37-4.22 (m, 5H), 4.02 (dp, J=21.6, 5.4, 4.9 Hz, 2H), 3.94-3.86 (m, 1H), 2.84 (s, 3H), 2.60-2.47 (m, 4H), 2.30 (dd, J=13.1, 7.0 Hz, 1H).

### Example 93: Synthesis of Compound 203

### Synthesis of Intermediate C114

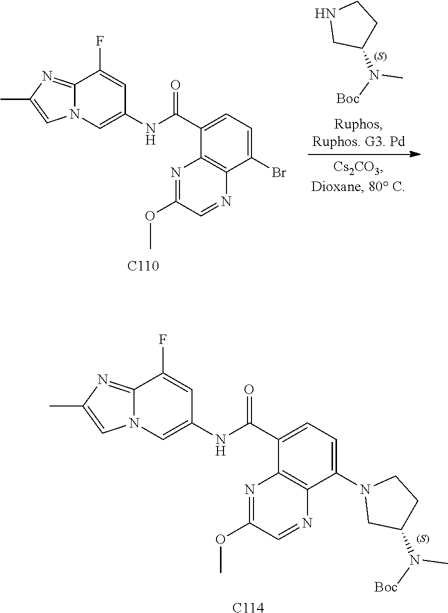

To a stirred mixture of 8-bromo-N-{8-fluoro-2-methylimidazo[1,2-a]pyridin-6-yl}-3-methoxyquinoxaline-5-carboxamide (100 mg, 0.232 mmol, 1 equiv) and tert-butyl (S)-methyl(pyrrolidin-3-yl)carbamate (55.86 mg, 0.278 mmol, 1.2 equiv) in dioxane (2 mL) was added Cs₂CO₃ (227.19 mg, 0.696 mmol, 3 equiv), Ruphos (21.69 mg, 0.046 mmol, 0.2 equiv), and RuPhos Palladacycle Gen.3 (19.44 mg, 0.023 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl (S)-(1-(8-((8-fluoro-2-methylimidazo[1,2-a]pyridin-6-yl)carbamoyl)-2-methoxyquinoxalin-5-yl)pyrrolidin-3-yl)(methyl)carbamate (70 mg, 55%) as a solid. LCMS (ES, m/z): 550 [M+H]⁺.

### Synthesis of Compound 203

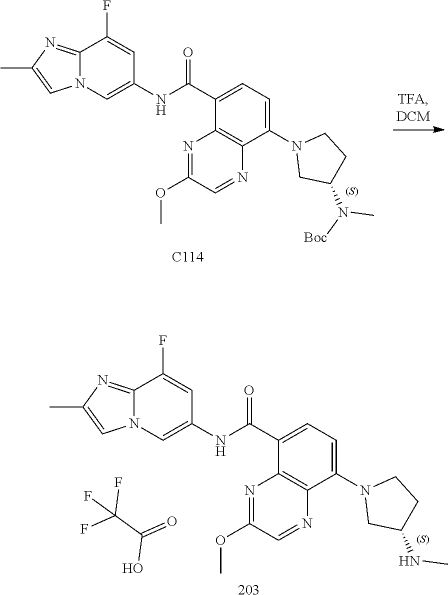

To a stirred solution of tert-butyl (S)—N-{1-[8-({8-fluoro-2-methylimidazo[1,2-a]pyridin-6-yl}carbamoyl)-2-methoxyquinoxalin-5-yl]pyrrolidin-3-yl}-N-methylcarbamate (70 mg, 0.127 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC with the following conditions (Condition 12, Gradient 1) to afford (S)—N-{8-fluoro-2-methylimidazo[1,2-a]pyridin-6-yl}-3-methoxy-8-[3-(methylamino)pyrrolidin-1-yl]quinoxaline-5-carboxamide; trifluoroacetic acid (5.5 mg, 8%) as a solid. LCMS (ES, m/z): 450 [M+H]⁺. ¹H NMR (400 MHz, DMSO-d₆) δ 11.74 (s, 1H), 9.41 (s, 1H), 8.79 (s, 2H), 8.57 (s, 1H), 8.42 (d, J=8.9 Hz, 1H), 8.14 (s, 1H), 7.62 (s, 1H), 6.88 (d, J=9.1 Hz, 1H), 4.26 (s, 3H), 4.24-4.17 (m, 1H), 4.12 (dd, J=12.5, 4.4 Hz, 1H), 4.05-3.92 (m, 2H), 3.88-3.77 (m, 1H), 2.72-2.65 (m, 3H), 2.44 (s, 3H), 2.42-2.35 (m, 1H), 2.22 (dd, J=12.6, 6.5 Hz, 1H).

### Example 94: Synthesis of Compound 204

### Synthesis of Intermediate C115

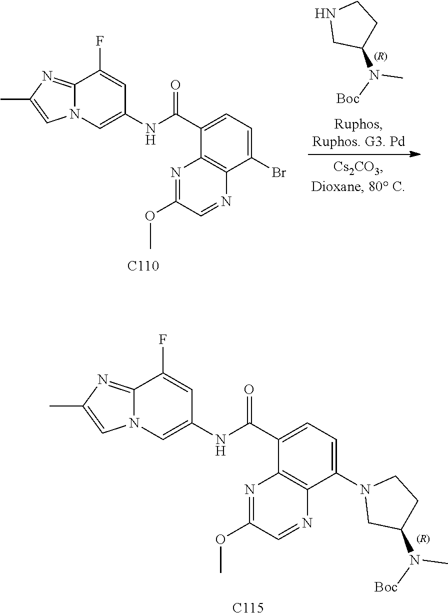

To a stirred mixture of 8-bromo-N-{8-fluoro-2-methylimidazo[1,2-a]pyridin-6-yl}-3-methoxyquinoxaline-5-carboxamide (100 mg, 0.232 mmol, 1 equiv) and tert-butyl (R)—N-methyl-N-(pyrrolidin-3-yl)carbamate (55.86 mg, 0.278 mmol, 1.2 equiv) in dioxane (2 mL) was added Cs₂CO₃ (227.19 mg, 0.696 mmol, 3 equiv), Ruphos (21.69 mg, 0.046 mmol, 0.2 equiv), and RuPhos Palladacycle Gen.3 (19.44 mg, 0.023 mmol, 0.2 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl (R)—N-{1-[8-({8-fluoro-2-methylimidazo[1,2-a]pyridin-6-yl}carbamoyl)-2-methoxyquinoxalin-5-yl]pyrrolidin-3-yl}-N-methylcarbamate (70 mg, 55%) as a solid. LCMS (ES, m/z): 550 [M+H]⁺.

### Synthesis of Compound 204

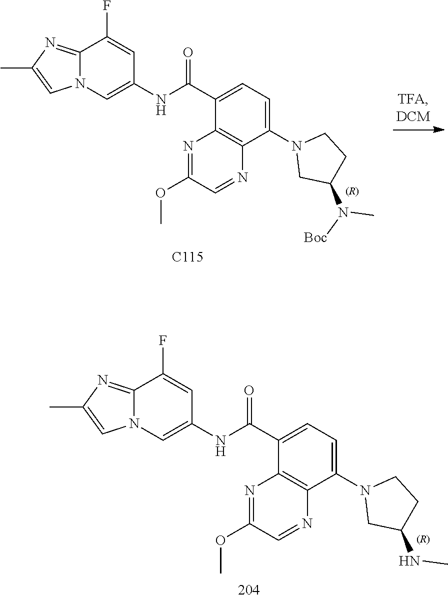

To a stirred solution of tert-butyl (R)—N-{1-[8-({8-fluoro-2-methylimidazo[1,2-a]pyridin-6-yl}carbamoyl)-2-methoxyquinoxalin-5-yl]pyrrolidin-3-yl}-N-methylcarbamate (70 mg, 0.127 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 12, Gradient 1) to afford (R)—N-{8-fluoro-2-methylimidazo[1,2-a]pyridin-6-yl}-3-methoxy-8-[3-(methylamino)pyrrolidin-1-yl]quinoxaline-5-carboxamide; trifluoroacetic acid (10.8 mg, 15%) as a solid. LCMS (ES, m/z): 450 [M+H]⁺. 1H NMR (400 MHz, DMSO-d₆) δ 11.74 (d, J=7.6 Hz, 1H), 9.41 (d, J=15.2 Hz, 1H), 8.80 (s, 2H), 8.57 (s, 1H), 8.42 (d, J=8.9 Hz, 1H), 8.14 (d, J=14.6 Hz, 1H), 7.60 (s, 1H), 6.88 (d, J=9.1 Hz, 1H), 4.52 (s, 3H), 4.25-4.17 (m, 1H), 4.12 (dd, J=12.5, 4.4 Hz, 1H), 4.05-3.90 (m, 2H), 3.84 (t, J=7.0 Hz, 1H), 2.72-2.65 (m, 3H), 2.44 (d, J=5.4 Hz, 3H), 2.42-2.33 (m, 1H), 2.21 (dt, J=12.2, 6.3 Hz, 1H).

### Example 95: Synthesis of Compound 206

### Synthesis of Compound 206

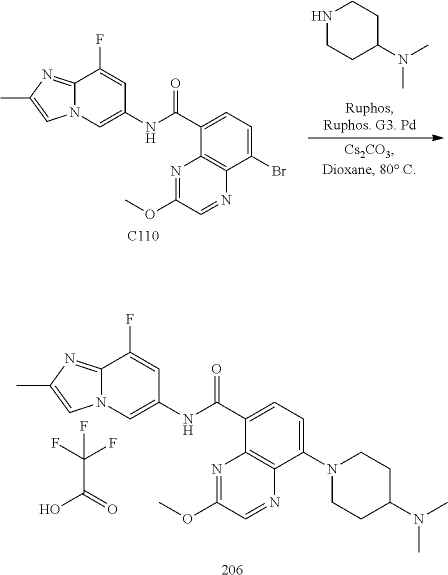

To a stirred mixture of 8-bromo-N-{8-fluoro-2-methylimidazo[1,2-a]pyridin-6-yl}-3-methoxyquinoxaline-5-carboxamide (45 mg, 0.105 mmol, 1 equiv) and N,N-dimethylpiperidin-4-amine (16.09 mg, 0.126 mmol, 1.2 equiv) in dioxane (1 mL) was added Cs₂CO₃ (102.24 mg, 0.315 mmol, 3 equiv), Ruphos (58.57 mg, 0.126 mmol, 1.2 equiv), and RuPhos Palladacycle Gen.3 (8.75 mg, 0.011 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1), followed by Prep-HPLC (Condition 12, Gradient 1) to afford 8-[4-(dimethylamino)piperidin-1-yl]-N-{8-fluoro-2-methylimidazo[1,2-a]pyridin-6-yl}-3-methoxyquinoxaline-5-carboxamide; trifluoroacetic acid (4.9 mg, 8%) as a solid. LCMS (ES, m/z): 478 [M+H]⁺. ¹H NMR (400 MHz, Methanol-d₄) δ 9.09 (d, J=1.7 Hz, 1H), 8.55 (d, J=9.4 Hz, 2H), 7.74 (d, J=2.9 Hz, 1H), 7.27-7.17 (m, 2H), 4.31 (s, 3H), 4.23 (d, J=12.3 Hz, 2H), 3.01 (t, J=12.0 Hz, 2H), 2.55 (s, 1H), 2.43 (dd, J=9.2, 1.0 Hz, 9H), 2.07 (d, J=12.2 Hz, 2H), 1.85 (q, J=12.8 Hz, 2H).

### Example 96: Synthesis of Compound

### Synthesis of Intermediate C116

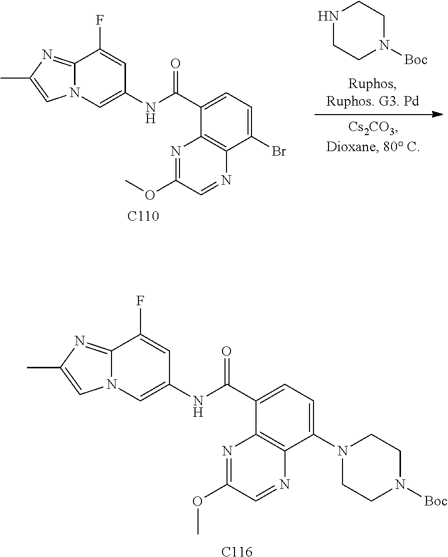 [SMILES: COc1cnc2c(Br)ccc(C(=O)Nc3cc(F)c4nc(C)cn4c3)c2n1 ; CC(C)(C)OC(=O)N1CCNCC1 ; "C110" ; COc1cnc2c(N3CCN(C(=O)OC(C)(C)C)CC3)ccc(C(=O)Nc3cc(F)c4nc(C)cn4c3)c2n1 ; "C116"]

To a stirred mixture of 8-bromo-N-{8-fluoro-2-methylimidazo[1,2-a]pyridin-6-yl}-3-methoxyquinoxaline-5-carboxamide (100 mg, 0.232 mmol, 1 equiv) and tert-butyl piperazine-1-carboxylate (51.95 mg, 0.278 mmol, 1.2 equiv) in dioxane (2 mL) was added Cs₂CO₃ (227.19 mg, 0.696 mmol, 3 equiv), Ruphos (21.69 mg, 0.046 mmol, 0.2 equiv), and RuPhos Palladacycle Gen.3 (19.44 mg, 0.023 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl-4-[8-({8-fluoro-2-methylimidazo[1,2-a]pyridin-6-yl}carbamoyl)-2-methoxyquinoxalin-5-yl]piperazine-1-carboxylate (60 mg, 48%) as a solid. LCMS (ES, m/z): 504 [M+H]⁺.

### Synthesis of Compound 207

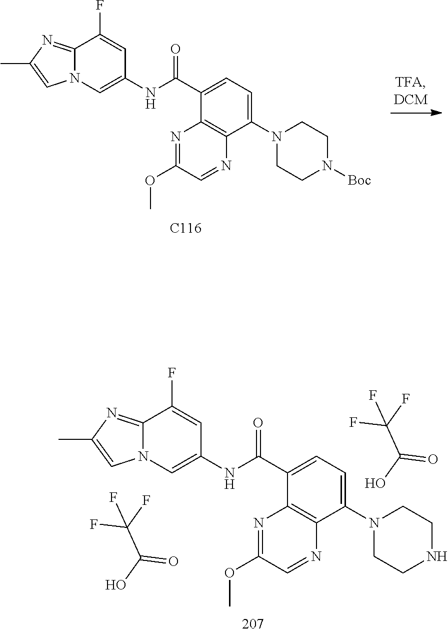

To a stirred solution of tert-butyl 4-[8-({8-fluoro-2-methylimidazo[1,2-a]pyridin-6-yl}carbamoyl)-2-methoxyquinoxalin-5-yl]piperazine-1-carboxylate (60 mg, 0.112 mmol, 1 equiv) in DCM (1 mL) was added TFA (1 mL, 13.463 mmol, 120.18 equiv) at room temperature. The resulting mixture was stirred for 2 h at room temperature. The resulting mixture was concentrated under reduced pressure to give a residue. The residue was purified by Prep-HPLC (Condition 12, Gradient 1) to afford N-{8-fluoro-2-methylimidazo[1,2-a]pyridin-6-yl}-3-methoxy-8-(piperazin-1-yl)quinoxaline-5-carboxamide; bis(trifluoroacetic acid) (21.3 mg, 29%) as a solid. LCMS (ES, m/z): 436 [M+H]⁺. ¹H NMR (400 MHz, Methanol-d₄) δ 9.40 (s, 1H), 8.66-8.59 (m, 2H), 8.03 (s, 1H), 7.71 (d, J=11.4 Hz, 1H), 7.31 (d, J=8.6 Hz, 1H), 4.34 (s, 3H), 3.85-3.78 (m, 4H), 3.57-3.50 (m, 4H), 2.56 (d, J=1.0 Hz, 3H).

### Example 97: Synthesis of Compound 195

### Synthesis of Intermediate C117

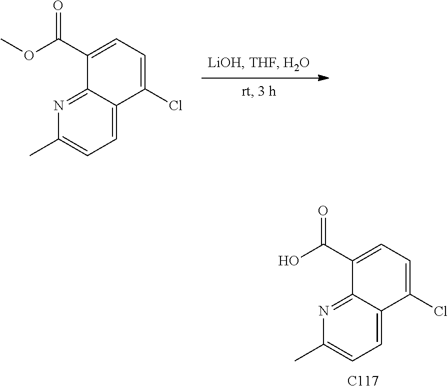

Methyl 5-chloro-2-methylquinoline-8-carboxylate (290.0 mg, 1.231 mmol, 1.0 equiv) tetrahydrofuran (3 mL), water (3 mL) and LiOH (235.7 mg, 9.848 mmol, 8.0 equiv) were combined at room temperature. The resulting mixture was stirred for 3 h at room temperature, diluted with H₂O (20 mL), acidified to pH 5 with HCl (aq.), and extracted with ethyl acetate (2×20 mL). The organic layers were combined, dried over anhydrous Na₂SO₄, and filtered. The filtrate was concentrated under reduced pressure to give a solid. LCMS (ES, m/z): 222 [M+H]⁺.

### Synthesis of Intermediate C118

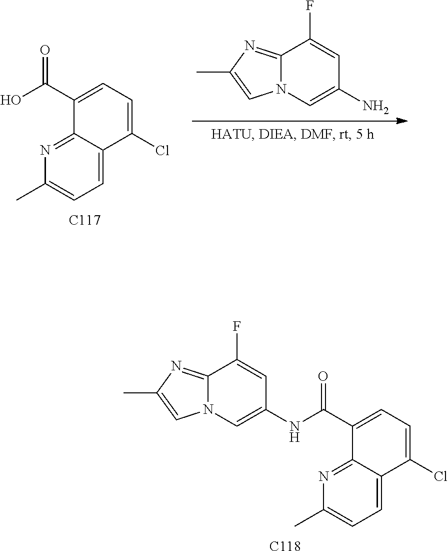

5-chloro-2-methylquinoline-8-carboxylic acid (280.0 mg, 1.263 mmol, 1.0 equiv), dimethylformamide (6 mL), DIEA (489.8 mg, 3.789 mmol, 3.0 equiv), HATU (609.0 mg, 2.526 mmol, 2.0 equiv) and 8-fluoro-2-methylimidazo[1,2-a]pyridin-6-amine (271.2 mg, 1.642 mmol, 1.3 equiv) were combined at room temperature. The resulting mixture was stirred for 5 h at room temperature, diluted with H₂O (30 mL), and extracted with ethyl acetate (2×50 mL). The combined organic layers were combined, dried over anhydrous Na₂SO₄, and filtered. The filtrate was concentrated under reduced pressure to give a residue. The residue was purified by silica gel column chromatography, eluted with PE/EA (1:1) to afford 5-chloro-N-{8-fluoro-2-methylimidazo[1,2-a]pyridin-6-yl}-2-methylquinoline-8-carboxamide (400 mg, 77%) as a solid. LCMS (ES, m/z):369 [M+H]⁺.

### Synthesis of Intermediate C119

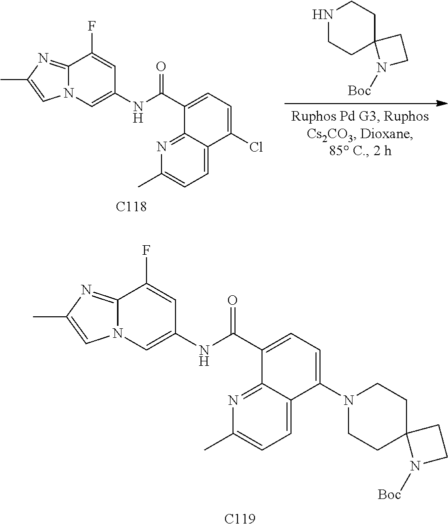 [SMILES: Cc1ccc2c(Cl)ccc(C(=O)Nc3cc(F)c4nc(C)cn4c3)c2n1 ; CC(C)(C)OC(=O)N1CCC12CCNCC2 ; "Ruphos Pd G3, RuphosCs2CO3, Dioxane,85° C., 2 h" ; "C118" ; Cc1ccc2c(N3CCC4(CC3)CCN4C(=O)OC(C)(C)C)ccc(C(=O)Nc3cc(F)c4nc(C)cn4c3)c2n1 ; "C119"]

5-chloro-N-{8-fluoro-2-methylimidazo[1,2-a]pyridin-6-yl}-2-methylquinoline-8-carboxamide (200.0 mg, 0.542 mmol, 1.0 equiv), Cs₂CO₃ (354.4 mg, 1.084 mmol, 2.0 equiv), tert-butyl 1,7-diazaspiro[3.5]nonane-1-carboxylate (184.1 mg, 0.813 mmol, 1.5 equiv), Ruphos (50.6 mg, 0.108 mmol, 0.2 equiv), Ruphos Pd G3 (45.3 mg, 0.054 mmol, 0.1 equiv), and dioxane (5 mL) were combined at room temperature. The resulting mixture was stirred for 2 h at 85° C. under nitrogen atmosphere, then concentrated under reduced pressure to give a residue. The residue was purified by silica gel column chromatography, eluted with PE/EA (1:2) to afford tert-butyl 7-[8-({8-fluoro-2-methylimidazo[1,2-a]pyridin-6-yl}carbamoyl)-2-methylquinolin-5-yl]-1,7-diazaspiro[3.5]nonane-1-carboxylate (150 mg, 45%) as a solid. LCMS (ES, m/z): 559 [M+H]⁺.

### Synthesis of Compound 195

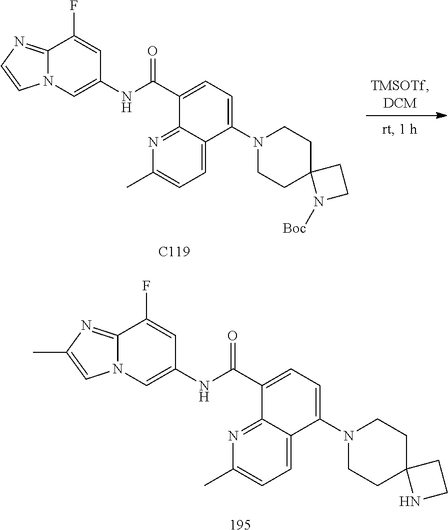

To a stirred mixture of tert-butyl 7-[8-({8-fluoro-2-methylimidazo[1,2-a]pyridin-6-yl}carbamoyl)-2-methylquinolin-5-yl]-1,7-diazaspiro[3.5]nonane-1-carboxylate (100.0 mg, 0.179 mmol, 1.0 equiv) in DCM (2 ml) was added DIEA (46.2 mg, 0.358 mmol, 2.0 equiv) and trimethylsilyl triflate (119.3 mg, 0.537 mmol, 3.0 equiv) dropwise at 0° C. The resulting mixture was stirred for 1 h at room temperature, then concentrated under reduced pressure to give a residue. The residue was purified by Prep-HPLC (Condition 10, Gradient 2) to afford 5-{1,7-diazaspiro[3.5]nonan-7-yl}-N-{8-fluoro-2-methylimidazo[1,2-a]pyridin-6-yl}-2-methylquinoline-8-carboxamide (23.7 mg, 29%) as a solid. LCMS (ES, m/z): 459 [M+H]⁺. ¹H NMR (400 MHz, DMSO-d₆) δ 13.77 (s, 1H), 9.25 (d, J=1.7 Hz, 1H), 8.57 (d, J=8.3 Hz, 1H), 8.49 (d, J=8.7 Hz, 1H), 7.93 (d, J=3.0 Hz, 1H), 7.59 (d, J=8.7 Hz, 1H), 7.31-7.22 (m, 2H), 3.39 (t, J=7.5 Hz, 2H), 3.14-3.01 (m, 2H), 3.01 (d, J=9.9 Hz, 2H), 2.88 (s, 3H), 2.36 (s, 3H), 2.09 (t, J=7.5 Hz, 2H), 2.02-1.85 (m, 4H).

### Example 98: Synthesis of Compound 196

### Synthesis of Intermediate C120

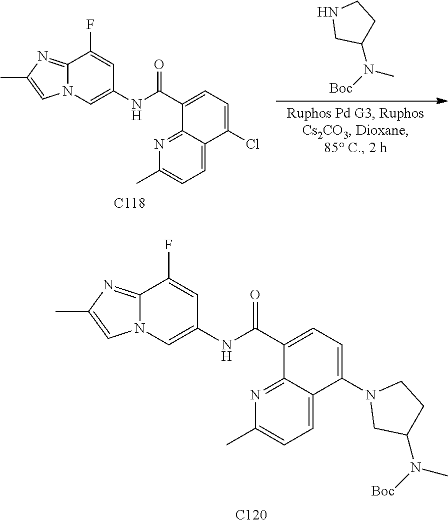

5-chloro-N-{8-fluoro-2-methylimidazo[1,2-a]pyridin-6-yl}-2-methylquinoline-8-carboxamide (100.0 mg, 0.271 mmol, 1.0 equiv), tert-butyl N-methyl-N-(pyrrolidin-3-yl)carbamate (81.4 mg, 0.407 mmol, 1.5 equiv), Ruphos (25.3 mg, 0.054 mmol, 0.2 equiv), RuPhos Palladacycle Gen.3 (12.6 mg, 0.027 mmol, 0.1 equiv), and dioxane (3 mL) were combined at room temperature. The resulting mixture was stirred for 2 h at 85° C. under nitrogen atmosphere, then concentrated under reduced pressure to give a residue. The residue was purified by silica gel column chromatography, eluted with EA to afford tert-butyl N-{1-[8-({8-fluoro-2-methylimidazo[1,2-a]pyridin-6-yl}carbamoyl)-2-methylquinolin-5-yl]pyrrolidin-3-yl}-N-methylcarbamate (90 mg, 57%) as a solid. LCMS (ES, m/z): 533 [M+H]⁺.

### Synthesis of Compound 196

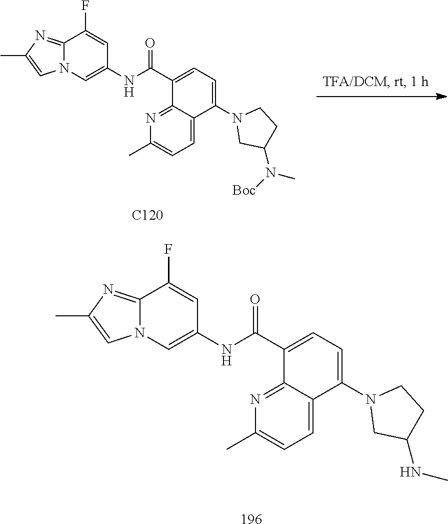

Tert-butyl N-{1-[8-({8-fluoro-2-methylimidazo[1,2-a]pyridin-6-yl}carbamoyl)-2-methylquinolin-5-yl]pyrrolidin-3-yl}-N-methylcarbamate (80.0 mg, 0.150 mmol, 1.0 equiv), DCM (1 mL), and TFA (1 mL) were combined at room temperature. The resulting mixture was stirred for 1 h at room temperature, then concentrated under reduced pressure to give a residue. The residue was purified by Prep-HPLC (Condition 10, Gradient 3) to afford N-{8-fluoro-2-methylimidazo[1,2-a]pyridin-6-yl}-2-methyl-5-[3-(methylamino)pyrrolidin-1-yl]quinoline-8-carboxamide 2,2,2-trifluoroacetate (10.9 mg, 17%) as a solid. LCMS (ES, m/z):433 [M+H]⁺. ¹H NMR (400 MHz, DMSO-d₆) δ 13.88 (s, 1H), 9.30 (d, J=1.6 Hz, 1H), 8.88-8.87 (m, 2H), 8.72 (d, J=8.8 Hz, 1H), 8.55 (d, J=8.5 Hz, 1H), 7.98 (d, J=3.0 Hz, 1H), 7.53 (d, J=8.8 Hz, 1H), 7.38 (d, J=12.0 Hz, 1H), 7.04 (d, J=8.6 Hz, 1H), 3.95-3.94 (m, 1H), 3.82 (dt, J=11.2, 6.3 Hz, 2H), 3.68 (dd, J=11.1, 3.9 Hz, 1H), 3.52 (q, J=8.0 Hz, 1H), 2.88 (s, 3H), 2.70 (s, 3H), 2.38 (s, 3H), 2.38-2.37 (m, 1H), 2.20 (dd, J=12.5, 6.4 Hz, 1H).

### Example 99: Synthesis of Compounds 190, 193, and 194

### Synthesis of Intermediate C121

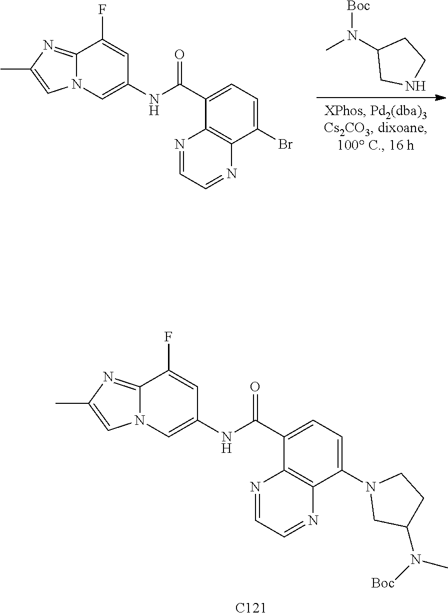

To a mixture of 8-bromo-N-{8-fluoro-2-methylimidazo[1,2-a]pyridin-6-yl}quinoxaline-5-carboxamide (115 mg, 0.287 mmol, 1 equiv) and tert-butyl N-methyl-N-(pyrrolidin-3-yl)carbamate (69.06 mg, 0.344 mmol, 1.2 equiv) in dioxane (10 mL) was added cesium carbonate (187.83 mg, 0.574 mmol, 2 equiv), XPhos (13.70 mg, 0.029 mmol, 0.1 equiv), and Pd₂(dba)₃ (26.31 mg, 0.029 mmol, 0.10 equiv). After stirring overnight at 100° C. under a nitrogen atmosphere, the resulting mixture was filtrated, and the filtrate concentrated under reduced pressure to give a residue. The residue was purified by silica gel column chromatography, eluted with PE:EA (1:2) to afford tert-butyl N-{1-[8-({8-fluoro-2-methylimidazo[1,2-a]pyridin-6-yl}carbamoyl)quinoxalin-5-yl]pyrrolidin-3-yl}-N-methylcarbamate (47 mg, 31%) as an oil. LCMS (ES, m/z): 520 [M+H]⁺.

### Synthesis of Compound 190

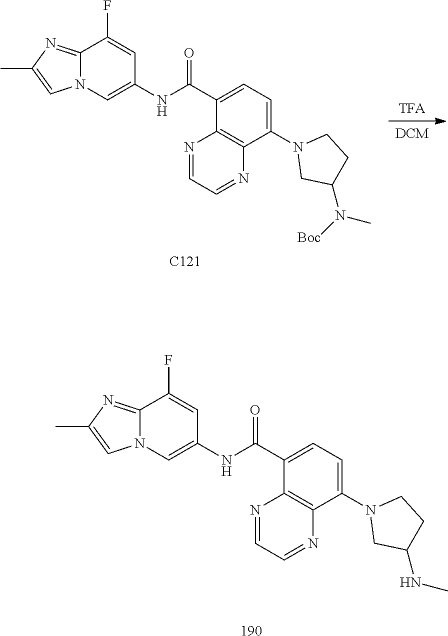

A solution of tert-butyl N-{1-[8-({8-fluoro-2-methylimidazo[1,2-a] pyridin-6-yl} carbamoyl) quinoxalin-5-yl]pyrrolidin-3-yl}-N-methylcarbamate (47 mg, 0.090 mmol, 1 equiv) in DCM (1 mL) was treated with trifluoroacetaldehyde (4 mL) for 1 h at room temperature. The resulting mixture was concentrated under reduced pressure to give a residue. The residue was purified by Prep-HPLC (Condition 12, Gradient 2, Gradient 3) to afford N-{8-fluoro-2-methylimidazo[1,2-a]pyridin-6-yl}-8-[3-(methylamino)pyrrolidin-1-yl]quinoxaline-5-carboxamide (19 mg, 50%) as a solid. LCMS (ES, m/z): 420 [M+H]⁺. ¹H NMR (300 MHz, DMSO-d₆) δ 12.88 (s, 1H), 9.54 (d, J=1.5 Hz, 1H), 9.09 (d, J=1.8 Hz, 1H), 8.96 (d, J=1.8 Hz, 1H), 8.55 (d, J=9.0 Hz, 1H), 8.22-8.15 (m, 1H), 8.01 (d, J=12.1 Hz, 1H), 6.98 (d, J=9.0 Hz, 1H), 4.24 (qd, J=13.0, 5.4 Hz, 2H), 4.02 (dd, J=18.7, 7.5 Hz, 2H), 3.91-3.79 (m, 1H), 2.70 (s, 3H), 2.70 (d, J=9.7 Hz, 3H), 2.45-2.36 (m, 1H), 2.25 (dd, J=12.6, 6.4 Hz, 1H).

### Synthesis of Compound 193 and Compound 194

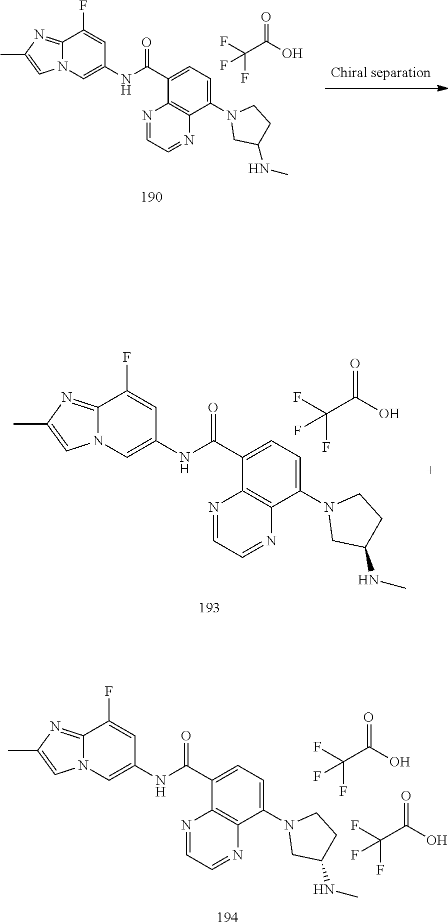

N-{8-fluoro-2-methylimidazo[1,2-a]pyridin-6-yl}-8-[3-(methylamino)pyrrolidin-1-yl]quinoxaline-5-carboxamide was purified by chiral prep-HPLC (Condition 2, Gradient 1).

Compound 193: RT=11.894. LCMS (ES, m/z): 420 [M+H]⁺. ¹H NMR (300 MHz, DMSO-d6) δ 12.75 (s, 1H), 9.41 (d, J=1.6 Hz, 1H), 9.10 (d, J=1.8 Hz, 1H), 8.96 (d, J=1.8 Hz, 1H), 8.87 (s, 1H), 8.56 (d, J=8.9 Hz, 1H), 8.05 (d, J=3.1 Hz, 1H), 7.76 (d, J=12.5 Hz, 1H), 6.99 (d, J=9.0 Hz, 1H), 4.34-4.25 (m, 1H), 4.20 (dd, J=12.9, 4.3 Hz, 1H), 4.07-3.95 (m, 2H), 3.87 (d, J=6.6 Hz, 1H), 2.69 (s, 3H), 2.41 (s, 4H), 1.24 (s, 1H). Compound 194: RT=15.341. LCMS (ES, m/z): 420 [M+H]⁺. ¹H NMR (300 MHz, DMSO-d6) δ 12.78 (s, 1H), 9.45 (s, 1H), 9.10 (d, J=1.8 Hz, 1H), 8.96 (d, J=1.8 Hz, 1H), 8.89 (s, 1H), 8.56 (d, J=8.8 Hz, 1H), 8.09 (s, 1H), 7.82 (d, J=12.3 Hz, 1H), 6.99 (d, J=9.0 Hz, 1H), 4.34-4.16 (m, 3H), 3.87 (d, J=7.9 Hz, 2H), 2.70 (s, 3H), 2.42 (s, 3H), 2.24 (dd, J=12.6, 6.6 Hz, 1H), 1.24 (s, 1H).

### Example 100: Synthesis of Compound 200

### Synthesis of Compound 200

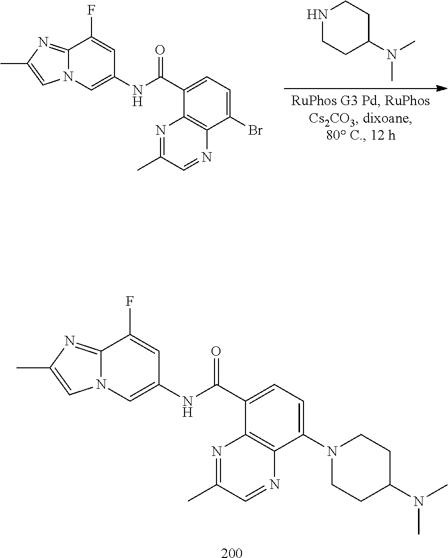

To a stirred mixture of 8-bromo-N-{8-fluoro-2-methylimidazo[1,2-a]pyridin-6-yl}-3-methylquinoxaline-5-carboxamide (80 mg, 0.193 mmol, 1 equiv) and N,N-dimethylpiperidin-4-amine (29.71 mg, 0.232 mmol, 1.2 equiv) in dioxane (1.5 mL) was added Cs₂CO₃ (188.77 mg, 0.579 mmol, 3 equiv), RuPhos (18.02 mg, 0.039 mmol, 0.2 equiv), and RuPhos Palladacycle Gen.3 (16.15 mg, 0.019 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1), followed by Prep-HPLC (Condition 11, Gradient 3) to afford 8-[4-(dimethylamino)piperidin-1-yl]-N-{8-fluoro-2-methylimidazo[1,2-a]pyridin-6-yl}-3-methylquinoxaline-5-carboxamide (11.5 mg, 13%) as a solid. LCMS (ES, m/z): 384 [M+H]⁺. 1H NMR (300 MHz, DMSO-d₆) δ 12.64 (s, 1H), 9.25 (d, J=1.6 Hz, 1H), 8.92 (s, 1H), 8.50 (d, J=8.6 Hz, 1H), 7.92 (d, J=3.0 Hz, 1H), 7.36-7.22 (m, 2H), 4.17 (d, J=12.1 Hz, 2H), 2.99 (t, J=11.8 Hz, 2H), 2.89 (s, 3H), 2.39-2.33 (m, 4H), 2.24 (s, 6H), 1.91 (d, J=12.4 Hz, 2H), 1.65 (d, J=10.0 Hz, 2H).

### Example 101: Synthesis of Compound 198

### Synthesis of Intermediate C122

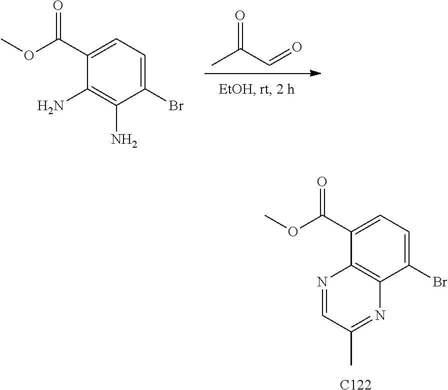

To a stirred solution of methyl 2,3-diamino-4-bromobenzoate (4 g, 16.321 mmol, 1 equiv) in ethanol (40 mL) was added 2-oxopropanal (1.41 g, 19.585 mmol, 1.2 equiv) dropwise at room temperature. The resulting mixture was stirred for 2 h at room temperature, then concentrated under reduced pressure to give a residue. The residue was purified by prep-HPLC (Condition 11: Gradient 1) to afford methyl 8-bromo-2-methylquinoxaline-5-carboxylate (320 mg, 7%) as a solid. LCMS (ES, m/z): 281 [M+H]⁺.

### Synthesis of Intermediate C123

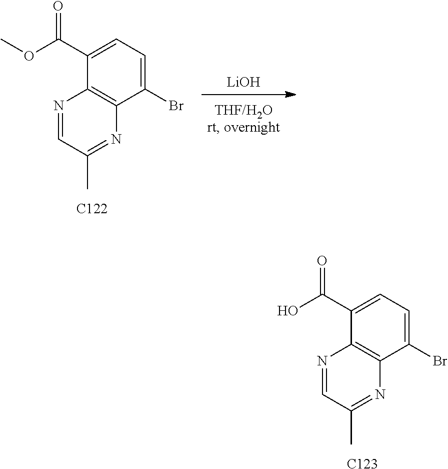 [SMILES: COC(=O)c1ccc(Br)c2nc(C)cnc12 ; "LiOH" ; "C122" ; Cc1cnc2c(C(=O)O)ccc(Br)c2n1 ; "C123"]

To a stirred solution of methyl 8-bromo-2-methylquinoxaline-5-carboxylate (320 mg, 1.138 mmol, 1 equiv) in THE (3 mL) and H₂O (1 mL) was added lithiumol (54.53 mg, 2.276 mmol, 2 equiv) at room temperature. The resulting mixture was stirred overnight at room temperature. The resulting mixture was concentrated under vacuum, diluted with H₂O (5 mL) and acidified to pH 3 with citric acid. A precipitate formed that was collected by filtration and dried under infrared light to afford 8-bromo-3-methylquinoxaline-5-carboxylic acid (200 mg, 66%) as a solid. LCMS (ES, m/z): 267 [M+H]⁺.

### Synthesis of Intermediate C124

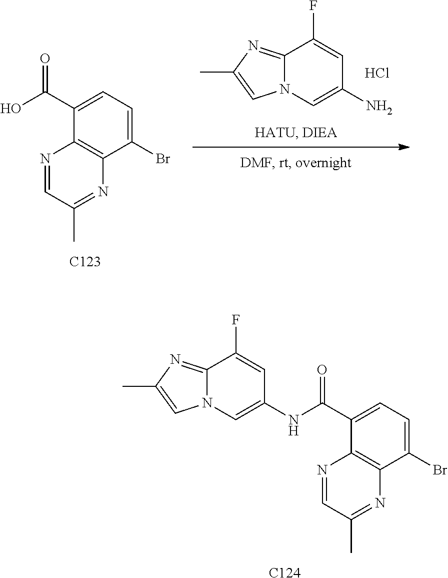

To a stirred mixture of 8-bromo-2-methylquinoxaline-5-carboxylic acid (200 mg, 0.749 mmol, 1 equiv) and HATU (341.68 mg, 0.899 mmol, 1.2 equiv) in DMF (4 mL) was added DIEA (267.61 mg, 2.072 mmol, 3.5 equiv) and 8-fluoro-2-methylimidazo[1,2-a]pyridin-6-amine hydrochloride (181.18 mg, 0.899 mmol, 1.2 equiv) at room temperature. The resulting mixture was stirred overnight at room temperature, then filtered. The filter cake was washed with Et₂O (2×5 mL) and dried under infrared light to afford 8-bromo-N-{8-fluoro-2-methylimidazo[1,2-a]pyridin-6-yl}-2-methylquinoxaline-5-carboxamide (150 mg, 48%) as a solid. LCMS (ES, m/z): 414 [M+H]

### Synthesis of Intermediate C125

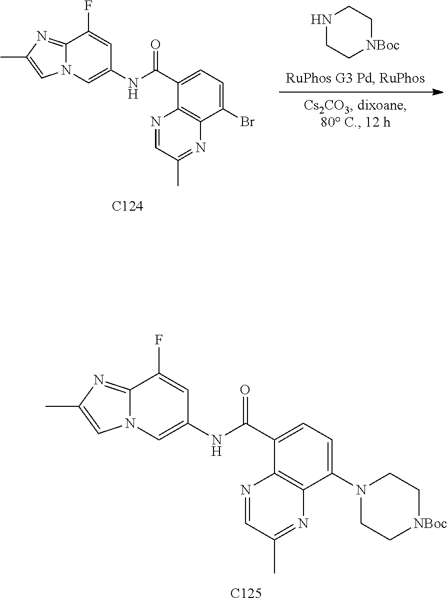

To a stirred mixture of 8-bromo-N-{8-fluoro-2-methylimidazo[1,2-a]pyridin-6-yl}-2-methylquinoxaline-5-carboxamide (65 mg, 0.157 mmol, 1 equiv) and tert-butyl piperazine-1-carboxylate (35.07 mg, 0.188 mmol, 1.2 equiv) in dioxane (1.3 mL) was added Cs₂CO₃ (153.38 mg, 0.471 mmol, 3 equiv), RuPhos (14.64 mg, 0.031 mmol, 0.2 equiv), and RuPhos Palladacycle Gen.3 (13.12 mg, 0.016 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl 4-[8-({8-fluoro-2-methylimidazo[1,2-a]pyridin-6-yl}carbamoyl)-3-methylquinoxalin-5-yl]piperazine-1-carboxylate (60 mg, 74%) as a solid. LCMS (ES, m/z): 520 [M+H]⁺.

### Synthesis of Compound 198

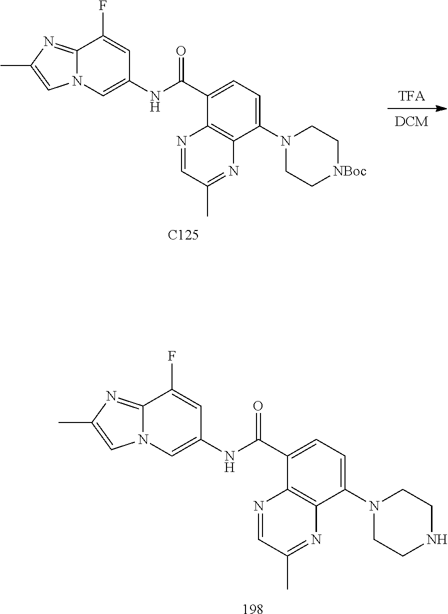

To a stirred solution of tert-butyl 4-[8-({8-fluoro-2-methylimidazo[1,2-a]pyridin-6-yl}carbamoyl)-3-methylquinoxalin-5-yl]piperazine-1-carboxylate (60 mg, 0.115 mmol, 1 equiv) in DCM (1 mL) was added trifluoroacetaldehyde (1 mL) at room temperature. The resulting mixture was stirred for 2 h at room temperature, then concentrated under reduced pressure to give a residue. The residue was purified by Prep-HPLC (Condition 11, Gradient 3) to afford N-{8-fluoro-2-methylimidazo[1,2-a]pyridin-6-yl}-2,3-dimethyl-8-(piperazin-1-yl)quinoxaline-5-carboxamide (12 mg, 24%) as a solid. LCMS (ES, m/z): 420 [M+H]⁺. ¹H NMR (400 MHz, DMSO-d₆) δ 12.67 (s, 1H), 9.44 (s, 1H), 8.98 (d, J=11.6 Hz, 3H), 8.55 (d, J=8.4 Hz, 1H), 8.12 (s, 1H), 7.67 (s, 1H), 7.40 (d, J=8.6 Hz, 1H), 3.77 (t, J=5.2 Hz, 4H), 3.39 (s, 4H), 2.92 (s, 3H), 2.44 (s, 3H).

### Example 102: Synthesis of Compound 208

### Synthesis of Compound 208

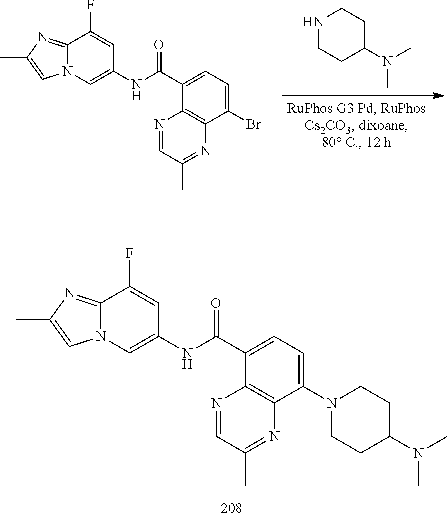

To a stirred mixture of 8-bromo-N-{8-fluoro-2-methylimidazo[1,2-a]pyridin-6-yl}-2-methylquinoxaline-5-carboxamide (40 mg, 0.097 mmol, 1 equiv) and N,N-dimethylpiperidin-4-amine (14.86 mg, 0.116 mmol, 1.2 equiv) in dioxane (1 mL) was added Cs₂CO₃ (94.39 mg, 0.291 mmol, 3 equiv), RuPhos (9.01 mg, 0.019 mmol, 0.2 equiv), and RuPhos Palladacycle Gen.3 (8.08 mg, 0.010 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1), followed by Prep-HPLC (Condition 12, Gradient 4) to afford 8-[4-(dimethylamino)piperidin-1-yl]-N-{8-fluoro-2-methylimidazo[1,2-a]pyridin-6-yl}-2-methylquinoxaline-5-carboxamide; bis(trifluoroacetic acid) (11.9 mg, 18%) as a solid. LCMS (ES, m/z): 462 [M+H]⁺. ¹H NMR (400 MHz, Methanol-d₄) δ 9.52 (d, J=1.5 Hz, 1H), 9.00 (s, 1H), 8.66 (d, J=8.5 Hz, 1H), 8.04 (dd, J=2.5, 1.2 Hz, 1H), 7.98 (dd, J=11.6, 1.5 Hz, 1H), 7.38 (d, J=8.5 Hz, 1H), 4.41 (d, J=12.4 Hz, 2H), 3.57-3.47 (m, 1H), 3.15-3.04 (m, 2H), 2.99 (s, 6H), 2.87 (s, 3H), 2.57 (d, J=1.0 Hz, 3H), 2.29 (d, J=12.0 Hz, 2H), 2.12 (qd, J=12.1, 4.0 Hz, 2H).

### Example 103: Synthesis of Compound 209

### Synthesis of Intermediate C126

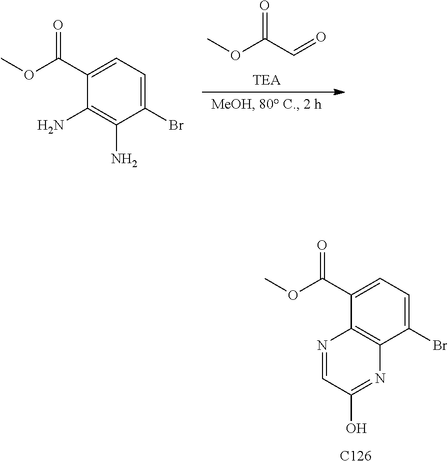

To a stirred solution of methyl 2,3-diamino-4-bromobenzoate (10 g, 40.804 mmol, 1 equiv), TEA (4.95 g, 48.965 mmol, 1.2 equiv) in ethanol (40 mL) was added methyl 2-oxoacetate (4.31 g, 48.965 mmol, 1.2 equiv) dropwise at room temperature. The resulting mixture was stirred for 2 h at 80° C., then concentrated under reduced pressure to give a residue. The residue was purified by prep-HPLC (Condition 11, Gradient 2) to afford methyl 8-bromo-2-hydroxyquinoxaline-5-carboxylate (1.5 g, 13%) as a solid. LCMS (ES, m/z): 283[M+H]⁺.

### Synthesis of Intermediate C127

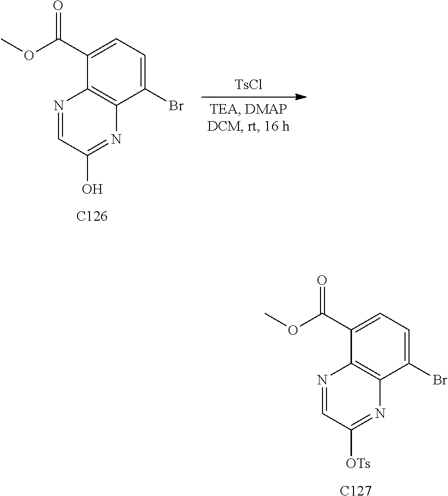 [SMILES: COC(=O)c1ccc(Br)c2nc(O)cnc12 ; "TsCl" ; "TEA, DMAPDCM, rt, 16 h" ; "C126" ; COC(=O)c1ccc(Br)c2nc(OS(=O)(=O)c3ccc(C)cc3)cnc12 ; "C127"]

To a stirred mixture of methyl 8-bromo-2-hydroxyquinoxaline-5-carboxylate (1.5 g, 5.299 mmol, 1 equiv) and TEA (0.80 g, 7.949 mmol, 1.5 equiv) in DCM (15 mL) was added DMAP (0.13 g, 1.060 mmol, 0.2 equiv) and P-toluenesulfonyl chloride (1.21 g, 6.359 mmol, 1.2 equiv) in portions at 0° C. The resulting mixture was stirred for 16 h at room temperature, then diluted with H₂O (10 mL) and extracted with DCM (2×30 mL). The organic layers were combined, washed with brine (1×30 mL), dried over anhydrous Na₂SO₄, and filtered. After filtration, the filtrate was concentrated under reduced pressure to afford methyl 8-bromo-2-[(4-methylbenzenesulfonyl)oxy]quinoxaline-5-carboxylate (1.48 g, 64%) as a solid. LCMS (ES, m/z): 437[M+H]⁺.

### Synthesis of Intermediate C128

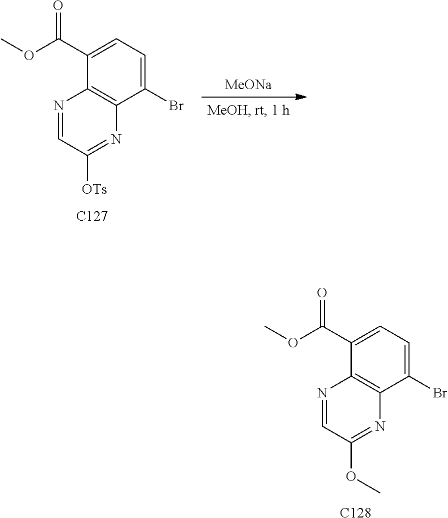 [SMILES: "MeONa" ; COC(=O)c1ccc(Br)c2nc(OS(=O)(=O)c3ccc(C)cc3)cnc12 ; "MeOH, rt, 1 h" ; "C127" ; COC(=O)c1ccc(Br)c2nc(OC)cnc12 ; "C128"]

To a stirred solution of methyl 8-bromo-2-[(4-methylbenzenesulfonyl)oxy]quinoxaline-5-carboxylate (1.48 g, 3.385 mmol, 1 equiv) in methanol (15 mL) was added MeONa (0.20 g, 3.724 mmol, 1.1 equiv) dropwise at room temperature. The resulting mixture was stirred for 2 h at room temperature, then concentrated under reduced pressure to give a residue. The residue was purified by silica gel column chromatography, eluted with PE/EA (4:1) to afford methyl 8-bromo-2-methoxyquinoxaline-5-carboxylate (850 mg, 85%) as a solid. LCMS (ES, m/z): 297[M+H]⁺.

### Synthesis of Intermediate C129

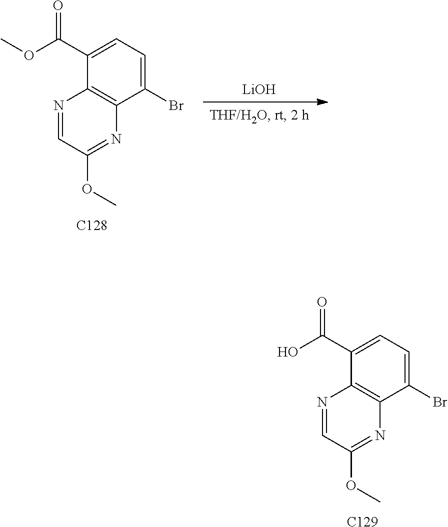 [SMILES: COC(=O)c1ccc(Br)c2nc(OC)cnc12 ; "LiOH" ; "THF/H2O, rt, 2 h" ; "C128" ; COc1cnc2c(C(=O)O)ccc(Br)c2n1 ; "C129"]

To a stirred solution of methyl 8-bromo-2-methoxyquinoxaline-5-carboxylate (850 mg, 2.861 mmol, 1 equiv) in THE (9 mL) and H₂O (3 mL) was added lithiumol (205.56 mg, 8.583 mmol, 3 equiv) at room temperature. The resulting mixture was stirred overnight at room temperature. The resulting mixture was concentrated under vacuum, diluted with H₂O (20 mL), and acidified to pH 3 with citric acid. A precipitate formed that was collected by filtration and dried under infrared light to afford 8-bromo-2-methoxyquinoxaline-5-carboxylic acid (730 mg, 90%) as a solid. LCMS (ES, m/z): 283[M+H]⁺.

### Synthesis of Intermediate C130

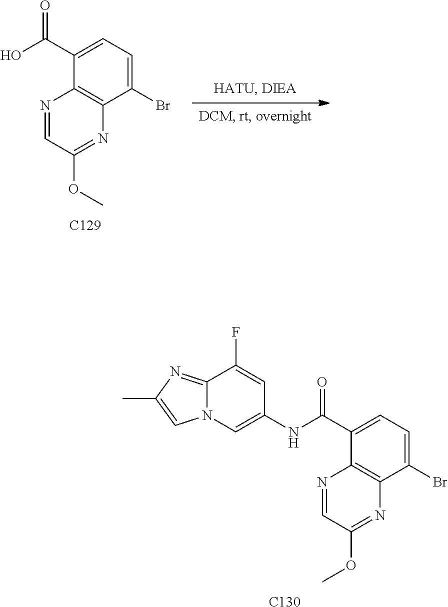 [SMILES: COc1cnc2c(C(=O)O)ccc(Br)c2n1 ; "DCM, rt, overnight" ; "HATU, DIEA" ; "C129" ; COc1cnc2c(C(=O)Nc3cc(F)c4nc(C)cn4c3)ccc(Br)c2n1 ; "C130"]

To a stirred mixture of 8-bromo-2-methoxyquinoxaline-5-carboxylic acid (730 mg, 2.579 mmol, 1 equiv) and HATU (1176.65 mg, 3.095 mmol, 1.2 equiv) in DCM (15 mL) was added DIEA (1499.84 mg, 11.606 mmol, 4.5 equiv) and 8-fluoro-2-methylimidazo[1,2-a]pyridin-6-amine hydrochloride (623.95 mg, 3.095 mmol, 1.2 equiv) at room temperature. The resulting mixture was stirred overnight at room temperature. The resulting mixture was concentrated under vacuum, diluted with H₂O (5 mL), then stirred for 30 min at room temperature. A precipitate formed that was collected by filtration and dried under infrared light to afford 8-bromo-N-{8-fluoro-2-methylimidazo[1,2-a]pyridin-6-yl}-2-methoxyquinoxaline-5-carboxamide (940 mg, 85%) as a solid. LCMS (ES, m/z): 430[M−H]⁻.

### Synthesis of Intermediate C131

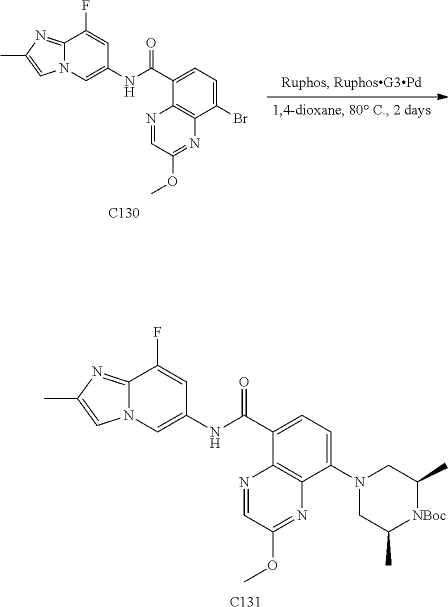

To a stirred mixture of 8-bromo-N-{8-fluoro-2-methylimidazo[1,2-a]pyridin-6-yl}-2-methoxyquinoxaline-5-carboxamide (120 mg, 0.279 mmol, 1 equiv), tert-butyl 2,6-dimethylpiperazine-1-carboxylate (71.73 mg, 0.335 mmol, 1.2 equiv), and Cs₂CO₃ (272.63 mg, 0.837 mmol, 3 equiv) in 1,4-dioxane (4 mL) was added Ruphos (13.02 mg, 0.028 mmol, 0.1 equiv) and RuPhos Palladacycle Gen.3 (23.33 mg, 0.028 mmol, 0.1 equiv) at room temperature under nitrogen atmosphere. The resulting mixture was stirred for 2 days at 80° C. under nitrogen atmosphere, then concentrated under vacuum to give a residue. The residue was purified by silica gel column chromatography, eluted with CH₂Cl₂/MeOH (50:1) to afford tert-butyl 4-[8-({8-fluoro-2-methylimidazo[1,2-a]pyridin-6-yl}carbamoyl)-3-methoxyquinoxalin-5-yl]-2,6-dimethylpiperazine-1-carboxylate (60 mg, 38%) as a solid. LCMS (ES, m/z): 564[M−H]⁻.

### Synthesis of Compound 209

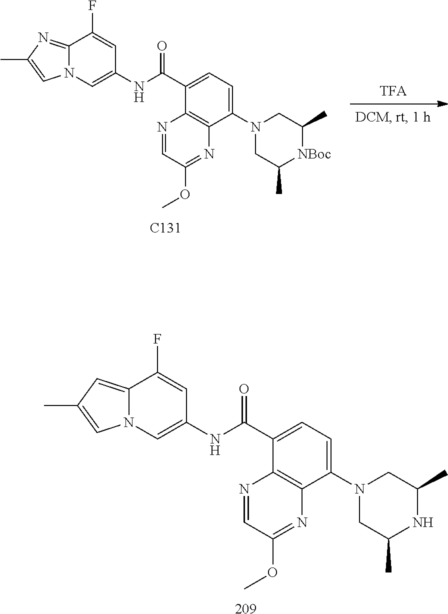

To a stirred solution of tert-butyl 4-[8-({8-fluoro-2-methylimidazo[1,2-a]pyridin-6-yl}carbamoyl)-3-methoxyquinoxalin-5-yl]-2,6-dimethylpiperazine-1-carboxylate (55 mg, 0.098 mmol, 1 equiv) in DCM (1 mL) was added TFA (1 mL) at room temperature. The resulting mixture was stirred for 1 h at room temperature, then concentrated under vacuum to give a residue. The residue was purified by Prep-HPLC (Condition 12, Gradient 1) to afford 8-(3,5-dimethylpiperazin-1-yl)-N-(8-fluoro-2-methylindolizin-6-yl)-2-methoxyquinoxaline-5-carboxamide; bis(trifluoroacetic acid) (23.9 mg, 35%) as a solid. LCMS (ES, m/z): 464[M+H]⁺. ¹H NMR (400 MHz, DMSO-d₆) δ 12.29 (d, J=2.8 Hz, 1H), 9.52-9.38 (m, 2H), 8.83 (d, J=1.3 Hz, 2H), 8.32-8.25 (m, 1H), 8.20-8.15 (m, 1H), 7.86 (d, J=12.0 Hz, 1H), 7.42 (d, J=8.4 Hz, 1H), 4.29-4.21 (m, 2H), 4.10 (s, 3H), 3.61 (d, J=10.4 Hz, 2H), 2.97 (dd, J=13.2, 11.0 Hz, 2H), 2.45 (s, 3H), 1.34 (d, J=6.5 Hz, 6H).

### Example 104: Synthesis of Compound 210

### Synthesis of Compound 210

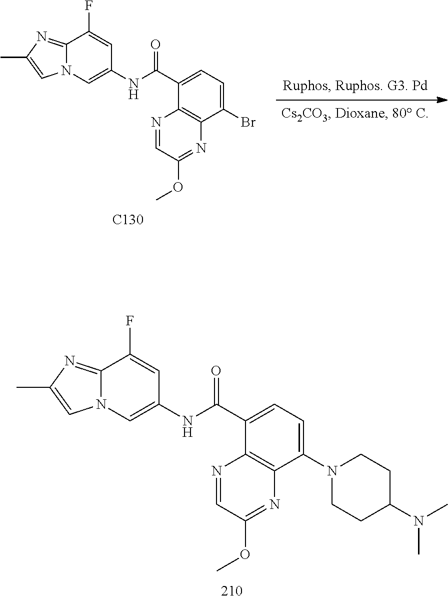

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv), N,N-dimethylpiperidin-4-amine (35.76 mg, 0.278 mmol, 1.2 equiv), and Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred for 2 days at 80° C. under nitrogen atmosphere, then concentrated under vacuum to give a residue. The residue was purified by silica gel column chromatography, eluted with CH₂Cl₂/MeOH (10:1), followed by Prep-HPLC (Condition 12, Gradient 1) to afford 8-[4-(dimethylamino)piperidin-1-yl]-N-{8-fluoro-2-methylimidazo[1,2-a]pyridin-6-yl}-2-methoxyquinoxaline-5-carboxamide; trifluoroacetic acid (22 mg, 16%) as a solid. LCMS (ES, m/z): 478[M+H]⁺. 1H NMR (400 MHz, DMSO-d₆) δ 12.23 (s, 1H), 9.59 (s, 1H), 9.41 (s, 1H), 8.83 (d, J=1.0 Hz, 1H), 8.30 (d, J=8.3 Hz, 1H), 8.07 (s, 1H), 7.68 (s, 1H), 7.35 (d, J=8.4 Hz, 1H), 4.23 (d, J=12.0 Hz, 2H), 4.12 (s, 3H), 3.44 (s, 1H), 2.93 (t, J=12.0 Hz, 2H), 2.85 (d, J=4.9 Hz, 6H), 2.41 (s, 3H), 2.19 (d, J=11.7 Hz, 2H), 1.99-1.88 (m, 2H).

### Example 105: Synthesis of Compound 211

### Synthesis of Intermediate C132

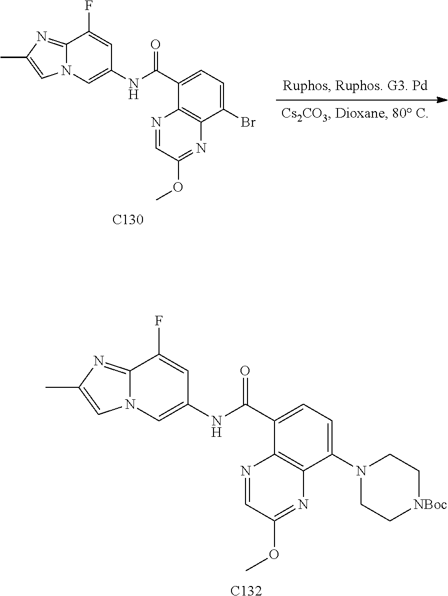 [SMILES: COc1cnc2c(C(=O)Nc3cc(F)c4nc(C)cn4c3)ccc(Br)c2n1 ; "Cs2CO3, Dioxane, 80° C." ; "Ruphos, Ruphos. G3. Pd" ; "C130" ; COc1cnc2c(C(=O)Nc3cc(F)c4nc(C)cn4c3)ccc(N3CCN(C(=O)OC(C)(C)C)CC3)c2n1 ; "C132"]

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv), tert-butyl piperazine-1-carboxylate (51.95 mg, 0.278 mmol, 1.2 equiv), and Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere, then concentrated under vacuum to give a residue. The residue was purified by silica gel column chromatography, eluted with CH₂Cl₂/MeOH (50:1) to afford tert-butyl 4-[8-({8-fluoro-2-methylimidazo[1,2-a]pyridin-6-yl}carbamoyl)-3-methoxyquinoxalin-5-yl]piperazine-1-carboxylate (70 mg, 56%) as a solid. LCMS (ES, m/z): 534[M−H]⁻.

### Synthesis of Compound 211

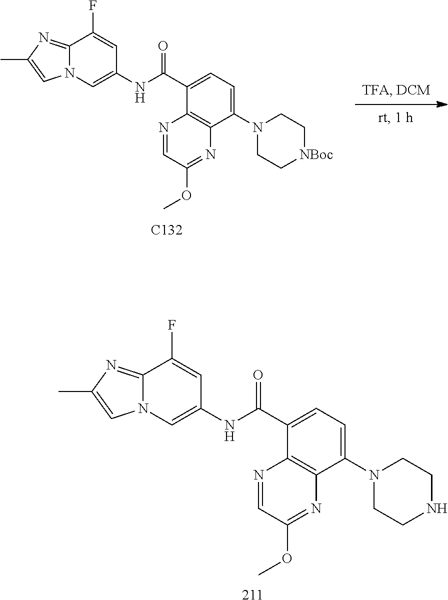

To a stirred solution of tert-butyl 4-[8-({8-fluoro-2-methylimidazo[1,2-a]pyridin-6-yl}carbamoyl)-3-methoxyquinoxalin-5-yl]piperazine-1-carboxylate (60 mg, 0.112 mmol, 1 equiv) in DCM (1 mL) was added TFA (1 mL) at room temperature. The resulting mixture was stirred for 1 h at room temperature, then concentrated under vacuum to give a residue. The residue was purified by Prep-HPLC (Condition 12, Gradient 1) to afford N-{8-fluoro-2-methylimidazo[1,2-a]pyridin-6-yl}-2-methoxy-8-(piperazin-1-yl)quinoxaline-5-carboxamide; trifluoroacetic acid (28.3 mg, 46%) as a solid. LCMS (ES, m/z): 436[M+H]⁺. ¹H NMR (400 MHz, DMSO-d₆) δ 12.20 (d, J=12.6 Hz, 1H), 9.47 (s, 1H), 9.03-8.81 (m, 3H), 8.29 (dd, J=8.3, 1.2 Hz, 1H), 8.14 (s, 1H), 7.77 (s, 1H), 7.41 (d, J=8.3 Hz, 1H), 4.11 (s, 3H), 3.73-3.66 (m, 4H), 3.43-3.38 (m, 4H), 2.43 (d, J=4.9 Hz, 3H).

### Example 106: Synthesis of Compound 212

### Synthesis of Intermediate C133

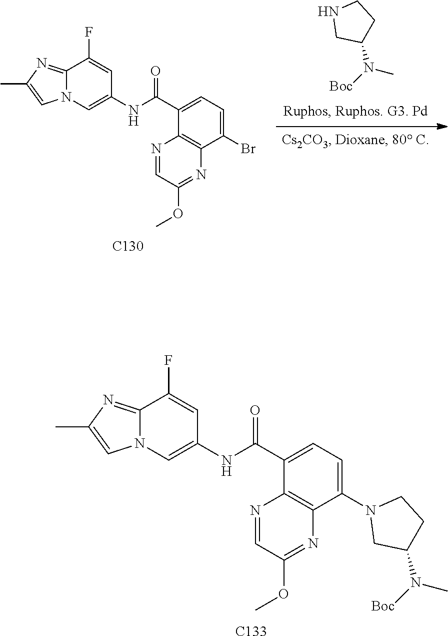

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv), tert-butyl (S)-methyl(pyrrolidin-3-yl)carbamate (55.86 mg, 0.278 mmol, 1.2 equiv), and Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere. The resulting mixture was concentrated under vacuum to give a residue. The residue was purified by silica gel column chromatography, eluted with CH₂Cl₂/MeOH (50:1) to afford tert-butyl (S)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (55 mg, 43%) as a solid. LCMS (ES, m/z): 550[M−H]⁻.

### Synthesis of Compound 212

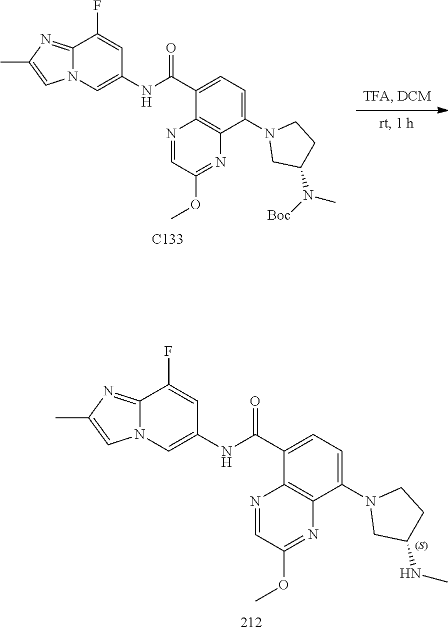

To a stirred solution of tert-butyl (S)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (50 mg, 0.091 mmol, 1 equiv) in DCM (1 mL) was added TFA (1 mL) at room temperature. The resulting mixture was stirred for 1 h at room temperature, then concentrated under vacuum to give a residue. The residue was purified by Prep-HPLC with the following conditions (Condition 12, Gradient 1) to afford (S)—N-{8-fluoro-2-methylimidazo[1,2-a]pyridin-6-yl}-2-methoxy-8-[3-(methylamino)pyrrolidin-1-yl]quinoxaline-5-carboxamide; trifluoroacetic acid (10.4 mg, 20%) as a solid. LCMS (ES, m/z): 450[M+H]⁺. ¹H NMR (400 MHz, DMSO-d₆) δ 12.50 (s, 1H), 9.43 (s, 1H), 8.81 (s, 3H), 8.34 (d, J=8.7 Hz, 1H), 8.08 (d, J=2.6 Hz, 1H), 7.75 (d, J=12.2 Hz, 1H), 6.96 (d, J=8.9 Hz, 1H), 4.26 (qd, J=12.4, 5.5 Hz, 2H), 4.09 (s, 3H), 3.96 (tt, J=12.7, 6.5 Hz, 2H), 3.82 (dt, J=11.0, 7.4 Hz, 1H), 2.72-2.65 (m, 3H), 2.44-2.33 (m, 4H), 2.21 (dq, J=13.1, 6.7 Hz, 1H).

### Example 107: Synthesis of Compound 213

### Synthesis of Intermediate C134

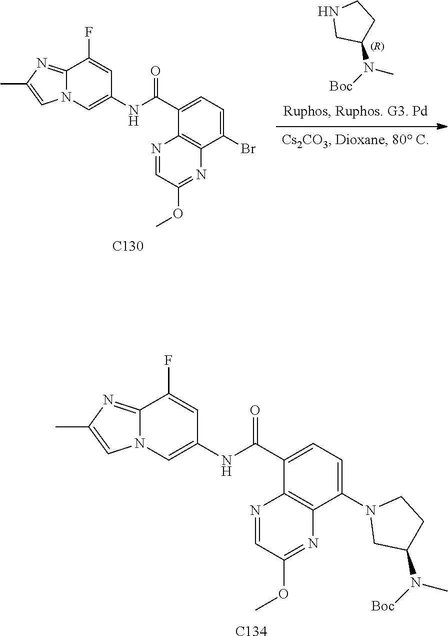

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv), tert-butyl-(R)-methyl(pyrrolidin-3-yl)carbamate (55.86 mg, 0.278 mmol, 1.2 equiv), and Cs₂CO₃ (227.19 mg, 0.696 mmol, 3 equiv) in 1,4-dioxane (3 mL) was added RuPhos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere, then concentrated under vacuum to give a residue. The residue was purified by silica gel column chromatography, eluted with CH₂Cl₂/MeOH (50:1) to afford tert-butyl (R)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (60 mg, 43%) as a solid. LCMS (ES, m/z): 550[M−H]⁺.

### Synthesis of Compound 213

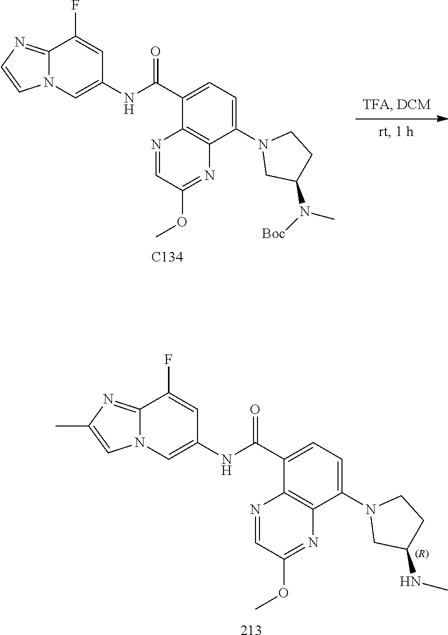

To a stirred solution of tert-butyl (R)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (50 mg, 0.091 mmol, 1 equiv) in DCM (1 mL) was added TFA (1 mL) at room temperature. The resulting mixture was stirred for 1 h at room temperature, then concentrated under vacuum to give a residue. The residue was purified by Prep-HPLC (Condition 12, Gradient 1) to afford (R)—N-(8-fluoro-2-methylimidazo[1,2-a]pyridin-6-yl)-2-methoxy-8-(3-(methylamino)pyrrolidin-1-yl)quinoxaline-5-carboxamide 2,2,2-trifluoroacetate (12.3 mg, 24%) as a solid. LCMS (ES, m/z): 450[M+H]⁺. 1H NMR (400 MHz, DMSO-d₆) δ 12.50 (s, 1H), 9.43 (s, 1H), 8.81 (s, 3H), 8.33 (d, J=8.7 Hz, 1H), 8.09 (d, J=2.5 Hz, 1H), 7.77 (d, J=12.2 Hz, 1H), 6.96 (d, J=8.8 Hz, 1H), 4.26 (qd, J=12.4, 5.6 Hz, 2H), 4.09 (s, 3H), 4.03-3.91 (m, 2H), 3.87-3.76 (m, 1H), 2.69 (d, J=10.3 Hz, 3H), 2.44-2.33 (m, 4H), 2.21 (dq, J=13.4, 6.9 Hz, 1H).

### Example 108: Synthesis of Compound 199

### Synthesisof Intermediate C135

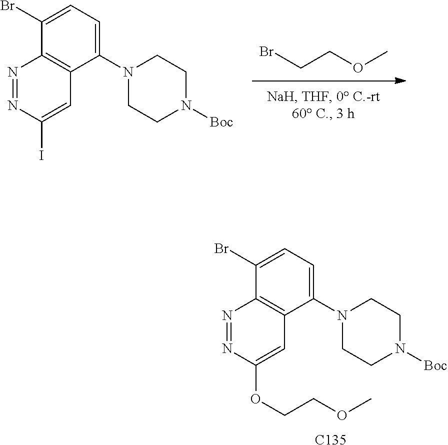

To a stirred solution of tert-butyl 4-(8-bromo-3-iodocinnolin-5-yl)piperazine-1-carboxylate (150.0 mg, 0.289 mmol, 1.0 equiv) in THE (3 mL) was added NaH (14 mg, 0.578 mmol, 2.0 equiv) dropwise at 0° C. The resulting mixture was stirred for 0.5 h at room temperature. To the reaction mixture was added 1-bromo-2-methoxyethane (59.8 mg, 0.433 mmol, 1.5 equiv) dropwise. The resulting mixture was heated to 65° C., and stirred for an additional 3 h at 65° C. The resulting mixture was quenched with ice-water (15 mL) and extracted with ethyl acetate (3×15 mL). The organic layers were combined, washed with water (3×15 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(8-bromo-3-(2-methoxyethoxy)cinnolin-5-yl)piperazine-1-carboxylate (65 mg, 48%) as a solid. LCMS (ES, m/z):467 [M+H]⁺.

### Synthesis of Intermediate C136

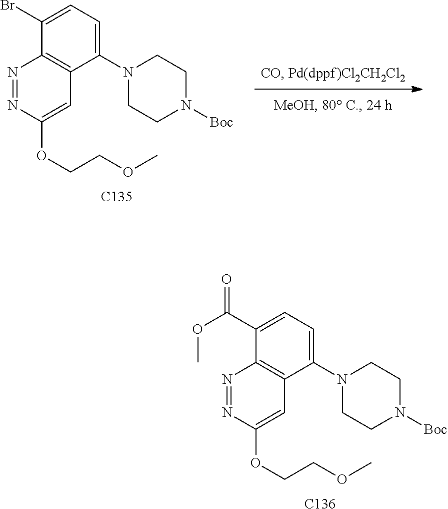 [SMILES: COCCOc1cc2c(N3CCN(C(=O)OC(C)(C)C)CC3)ccc(Br)c2nn1 ; "C135" ; COCCOc1cc2c(N3CCN(C(=O)OC(C)(C)C)CC3)ccc(C(=O)OC)c2nn1 ; "C136"]

To a solution of tert-butyl 4-[8-bromo-3-(2-methoxyethoxy)cinnolin-5-yl]piperazine-1-carboxylate (65.0 mg, 0.139 mmol, 1.0 equiv) in methanol (20 mL) was added Et₃N (42.2 mg, 0.417 mmol, 3.0 equiv) and Pd(dppf)Cl₂CH₂Cl₂ (10.2 mg, 0.014 mmol, 0.1 equiv) in a pressure tank. The reaction mixture was pressurized to 1 MPa with carbon monoxide at 80° C. for 16 h. The reaction mixture was cooled to room temperature, then filtered. The filtrate was concentrated under vacuum to give a residue. The residue was purified by silica gel column chromatography, eluted with PE/EA (1:1) to afford methyl 5-[4-(tert-butoxycarbonyl)piperazin-1-yl]-3-(2-methoxyethoxy)cinnoline-8-carboxylate (55 mg, 89%) as a solid. LCMS (ES, m/z):447 [M+H]⁺.

### Synthesis of Intermediate C137

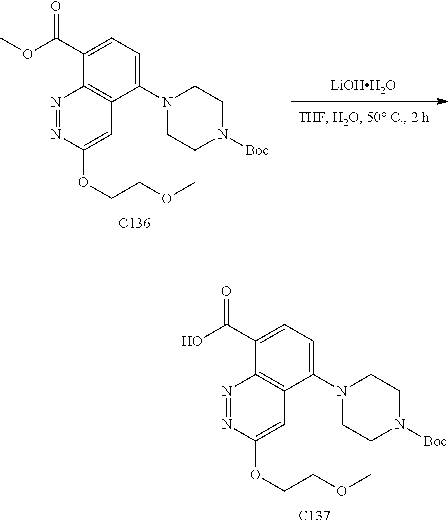 [SMILES: COCCOc1cc2c(N3CCN(C(=O)OC(C)(C)C)CC3)ccc(C(=O)OC)c2nn1 ; "LiOH·H2O" ; "C136" ; COCCOc1cc2c(N3CCN(C(=O)OC(C)(C)C)CC3)ccc(C(=O)O)c2nn1 ; "C137"]

To a stirred mixture of methyl 5-[4-(tert-butoxycarbonyl)piperazin-1-yl]-3-(2-methoxyethoxy)cinnoline-8-carboxylate (55 mg, 0.123 mmol, 1 equiv) in THF (1 mL) was added lithiumol hydrate (41.4 mg, 0.984 mmol, 8 equiv) and water (1 mL) at room temperature. The resulting mixture was stirred for 2 h at 50° C., then cooled to room temperature. The resulting mixture was diluted with water (3 mL), acidified to pH 6 with HCl (1N), and extracted with ethyl acetate (3×3 mL). The organic layers were combined, washed with water (3×3 mL), dried over anhydrous Na₂SO₄, and filtered. After filtration, the filtrate was concentrated under reduced pressure to afford 5-[4-(tert-butoxycarbonyl)piperazin-1-yl]-3-(2-methoxyethoxy)cinnoline-8-carboxylic acid (65 mg, 98%) as a solid. LCMS (ES, m/z):433 [M+H]⁺.

### Synthesis of Intermediate C138

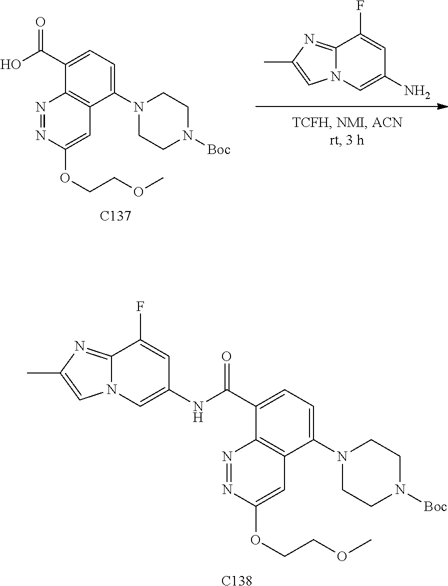 [SMILES: COCCOc1cc2c(N3CCN(C(=O)OC(C)(C)C)CC3)ccc(C(=O)O)c2nn1 ; Cc1cn2cc(N)cc(F)c2n1 ; "C137" ; COCCOc1cc2c(N3CCN(C(=O)OC(C)(C)C)CC3)ccc(C(=O)Nc3cc(F)c4nc(C)cn4c3)c2nn1 ; "C138"]

To a stirred mixture of 5-[4-(tert-butoxycarbonyl)piperazin-1-yl]-3-(2-methoxyethoxy)cinnoline-8-carboxylic acid (50.0 mg, 0.116 mmol, 1.0 equiv) and 8-fluoro-2-methylimidazo[1,2-a]pyridin-6-amine (22.9 mg, 0.139 mmol, 1.2 equiv) in acetonitrile (0.5 mL) was added NMI (38.0 mg, 0.464 mmol, 4.0 equiv) and TCFH (38.9 mg, 0.139 mmol, 1.2 equiv) at room temperature. The resulting mixture was stirred for 3 h at room temperature, then diluted with water (1 mL) and extracted with ethyl acetate (3×2 mL). The organic layers were combined, washed with water (3×2 mL), dried over anhydrous Na₂SO₄, and filtered. After filtration, the filtrate was concentrated under reduced pressure to give a residue. The residue was purified by silica gel column chromatography, eluted with EA to afford tert-butyl 4-[8-({8-fluoro-2-methylimidazo[1,2-a]pyridin-6-yl}carbamoyl)-3-(2-methoxyethoxy)cinnolin-5-yl]piperazine-1-carboxylate (30 mg, 45%) as a solid. LCMS (ES, m/z):580[M+H]⁺.

### Synthesis of Compound 199

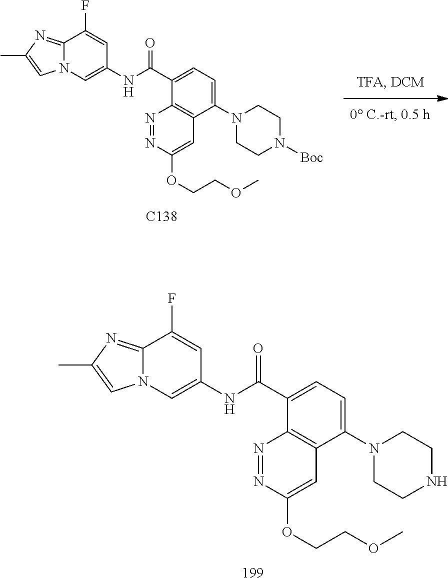 [SMILES: COCCOc1cc2c(N3CCN(C(=O)OC(C)(C)C)CC3)ccc(C(=O)Nc3cc(F)c4nc(C)cn4c3)c2nn1 ; "C138" ; COCCOc1cc2c(N3CCNCC3)ccc(C(=O)Nc3cc(F)c4nc(C)cn4c3)c2nn1 ; "199"]

To a stirred solution of tert-butyl 4-[8-({8-fluoro-2-methylimidazo[1,2-a]pyridin-6-yl}carbamoyl)-3-(2-methoxyethoxy)cinnolin-5-yl]piperazine-1-carboxylate (25.0 mg, 0.043 mmol, 1.0 equiv) in DCM (0.5 mL) was added TFA (0.05 mL) dropwise at 0° C. The resulting mixture was stirred for 30 min at room temperature, then concentrated under vacuum to give a residue. The residue was purified by Prep-HPLC (Condition 11, Gradient 3) to afford N-{8-fluoro-2-methylimidazo[1,2-a]pyridin-6-yl}-3-(2-methoxyethoxy)-5-(piperazin-1-yl)cinnoline-8-carboxamide (3 mg, 15%) as a solid. LCMS (ES, m/z):480 [M+H]⁺. ¹H NMR (300 MHz, DMSO-d₆) δ 11.88 (s, 1H), 9.21 (s, 1H), 8.27 (d, J=7.9 Hz, 1H), 7.95 (s, 1H), 7.64 (s, 1H), 7.36-7.25 (m, 2H), 4.78-4.77 (m, 2H), 3.83-3.82 (m, 2H), 3.32 (s, 3H), 3.03-2.99 (m, 8H), 2.36 (s, 3H).

### Example 109: Synthesis of Compound 158

### Synthesis of Compound 158

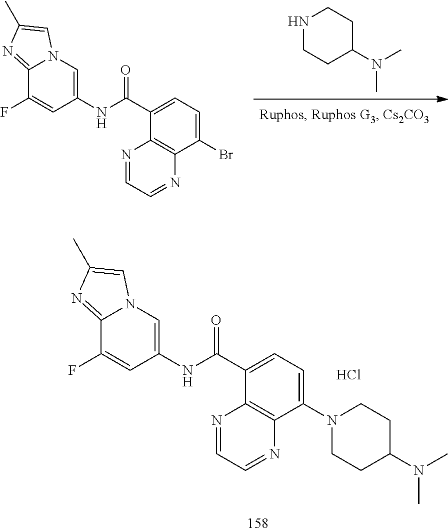

A mixture of 8-bromo-N-{8-fluoro-2-methylimidazo[1,2-a]pyridin-6-yl}quinoxaline-5-carboxamide (600 mg, 1.499 mmol, 1 equiv), N,N-dimethylpiperidin-4-amine (288.34 mg, 2.248 mmol, 1.5 equiv), RuPhos (34.98 mg, 0.075 mmol, 0.05 equiv), RuPhos Palladacycle Gen.3 (62.70 mg, 0.075 mmol, 0.05 equiv), and Cs₂CO₃ (1.47 g, 4.497 mmol, 3 equiv) in DMA (6 mL) was stirred for 2 h at 100° C. under N₂ atmosphere. The resulting mixture was concentrated under reduced pressure to give a residue. The residue was purified by silica gel column chromatography, eluted with DCM:MeOH to give a residue. The residue was dissolved in DCM (2 mL) and precipitated with petroleum ether (10 mL) to afford 8-(4-(dimethylamino)piperidin-1-yl)-N-(8-fluoro-2-methylimidazo[1,2-a]pyridin-6-yl)quinoxaline-5-carboxamide; hydrogen chloride salt (130 mg, 22%) as a solid. LCMS (ES, m/z): 448 [M+H]⁺. ¹H NMR (300 MHz, Methanol-d4) δ 9.64 (d, J=1.5 Hz, 1H), 9.12 (d, J=1.8 Hz, 1H), 9.04 (s, 1H), 8.76 (d, J=8.5 Hz, 1H), 8.22 (dd, J=11.6, 1.4 Hz, 1H), 8.15 (s, 1H), 7.45 (d, J=8.5 Hz, 1H), 4.43 (d, J=12.5 Hz, 2H), 3.63-3.49 (m, 1H), 3.17 (t, J=12.1 Hz, 2H), 2.99 (s, 6H), 2.61 (s, 3H), 2.32 (d, J=11.8 Hz, 2H), 2.22-2.09 (m, 2H).

### Example 110: Synthesis of Compound 214

### Synthesis of Intermediate C139

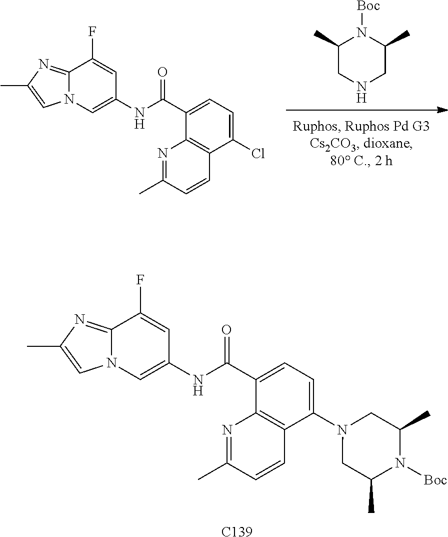

To a solution of 5-chloro-N-{8-fluoro-2-methylimidazo[1,2-a] pyridin-6-yl}-2-methylquinoline-8-carboxamide (120.0 mg, 0.325 mmol, 1.0 equiv) and tert-butyl (2R,6S)-2,6-dimethylpiperazine-1-carboxylate (104.6 mg, 0.488 mmol, 1.5 equiv) in dioxane (5 mL) was added Cs₂CO₃ (265.8 mg, 0.813 mmol, 2.5 equiv), RuPhos Palladacycle Gen.3 (27.2 mg, 0.033 mmol, 0.1 equiv), and Ruphos (30.3 mg, 0.065 mmol, 0.2 equiv). After stirring for 2 h at 85° C. under a nitrogen atmosphere, the resulting mixture was concentrated under reduced pressure to give a residue. The residue was purified by silica gel column chromatography, eluted with CH₂Cl₂/MeOH (10:1) to afford tert-butyl (2R,6S)-4-[8-({8-fluoro-2-methylimidazo[1,2-a]pyridin-6-yl}carbamoyl)-2-methylquinolin-5-yl]-2,6-dimethylpiperazine-1-carboxylate (150 mg, 72%) as a solid. LCMS (ES, m/z): 547 [M+H]⁺.

### Synthesis of Compound 214

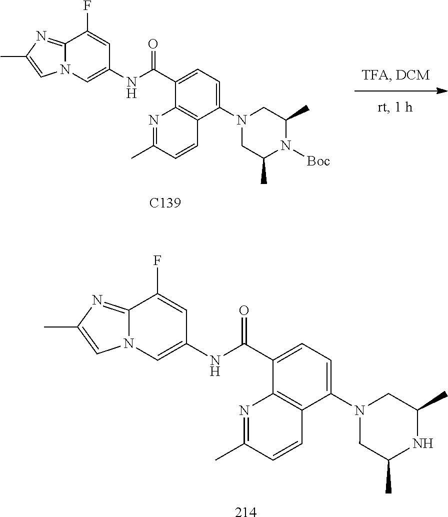

To a stirred solution of tert-butyl (2R,6S)-4-[8-({8-fluoro-2-methylimidazo[1,2-a] pyridin-6-yl}carbamoyl)-2-methylquinolin-5-yl]-2,6-dimethylpiperazine-1-carboxylate (80.0 mg, 0.146 mmol, 1.0 equiv) in DCM (2 mL) was added TFA (0.5 mL) dropwise at room temperature. The resulting mixture was stirred for 1 h at room temperature. The reaction was monitored by LCMS. The resulting mixture was concentrated under reduced pressure to give a residue. The residue was purified by Prep-HPLC (Condition 10, Gradient 2) to afford 5-[(3R,5S)-3,5-dimethylpiperazin-1-yl]-N-{8-fluoro-2-methylimidazo[1,2-a]pyridin-6-yl}-2-methylquinoline-8-carboxamide (13.0 mg, 20%) as a solid. LCMS (ES, m/z): 447 [M+H]⁺. ¹H NMR (400 MHz, DMSO-d₆) δ 13.78 (s, 1H), 9.26 (d, J=1.6 Hz, 1H), 8.58 (d, J=8.2 Hz, 1H), 8.54 (d, J=8.7 Hz, 1H), 7.93 (d, J=3.1 Hz, 1H), 7.60 (d, J=8.7 Hz, 1H), 7.32-7.23 (m, 2H), 3.29 (m, 2H), 3.19 (m, 2H), 2.89 (s, 3H), 2.45 (m, 2H), 2.36 (s, 3H), 1.06 (d, J=6.2 Hz, 6H).

### Example 111: Synthesis of Compound 215

### Synthesis of Compound 215

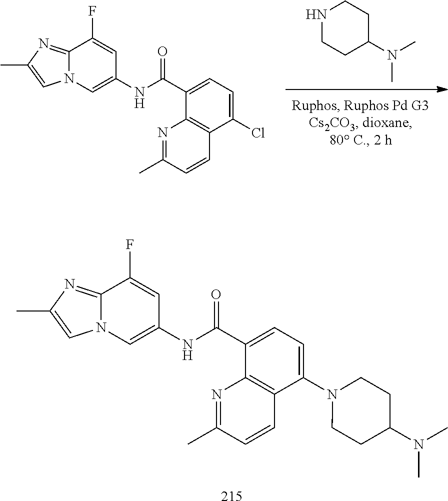

To a solution of 5-chloro-N-{8-fluoro-2-methylimidazo[1,2-a] pyridin-6-yl}-2-methylquinoline-8-carboxamide (120.0 mg, 0.325 mmol, 1.0 equiv) and N,N-dimethylpiperidin-4-amine (62.5 mg, 0.488 mmol, 1.5 equiv) in dioxane (5 mL) were added Cs₂CO₃ (265.8 mg, 0.813 mmol, 2.5 equiv) and Ruphos (30.3 mg, 0.065 mmol, 0.2 equiv), RuPhos Palladacycle Gen.3 (54.43 mg, 0.065 mmol, 0.2 equiv). After stirring for 2 h at 80° C. under a nitrogen atmosphere, the resulting mixture was concentrated under reduced pressure. The residue was purified silica gel column chromatography, eluted with CH₂Cl₂/MeOH (5:1) to afford 5-[4-(dimethylamino) piperidin-1-yl]-N-{8-fluoro-2-methylimidazo[1,2-a] pyridin-6-yl}-2-methylquinoline-8-carboxamide (41 mg, 27.09%) as a solid. LCMS (ES, m/z): 461 [M+H]⁺. ¹H NMR (300 MHz, Chloroform-d) δ 14.19 (s, 1H), 9.32 (d, J=1.6 Hz, 1H), 8.78 (d, J=8.2 Hz, 1H), 8.48 (d, J=8.6 Hz, 1H), 7.49-7.37 (m, 2H), 7.19 (d, J=8.2 Hz, 1H), 6.88 (dd, J=11.2, 1.7 Hz, 1H), 3.55 (d, J=12.1 Hz, 2H), 2.91-2.85 (m, 5H), 2.60-2.50 (m, 10H), 2.14 (d, J=12.5 Hz, 2H), 1.92 (m, 2H).

### Example 112: Synthesis of Compound 216

### Synthesis of Intermediate C140

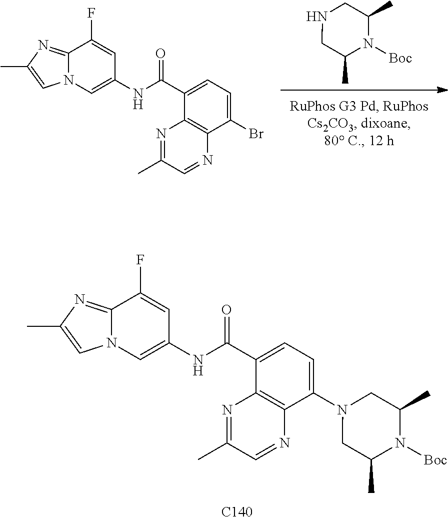 [SMILES: Cc1cnc2c(Br)ccc(C(=O)Nc3cc(F)c4nc(C)cn4c3)c2n1 ; C[C@@H]1CNC[C@H](C)N1C(=O)OC(C)(C)C ; Cc1cnc2c(N3C[C@@H](C)N(C(=O)OC(C)(C)C)[C@@H](C)C3)ccc(C(=O)Nc3cc(F)c4nc(C)cn4c3)c2n1 ; "C140"]

To a stirred mixture of 8-bromo-N-{8-fluoro-2-methylimidazo[1,2-a]pyridin-6-yl}-3-methylquinoxaline-5-carboxamide (40 mg, 0.097 mmol, 1 equiv) and tert-butyl (2R,6S)-2,6-dimethylpiperazine-1-carboxylate (24.83 mg, 0.116 mmol, 1.2 equiv) in dioxane (1 mL) was added Cs₂CO₃ (94.39 mg, 0.291 mmol, 3 equiv), RuPhos (9.01 mg, 0.019 mmol, 0.2 equiv) and RuPhos Palladacycle Gen.3 (8.08 mg, 0.010 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl (2R,6S)-4-[8-({8-fluoro-2-methylimidazo[1,2-a]pyridin-6-yl}carbamoyl)-2-methylquinoxalin-5-yl]-2,6-dimethylpiperazine-1-carboxylate (40 mg, 76%) as a solid. LCMS (ES, m/z): 548 [M+H]⁺.

### Synthesis of Compound 216

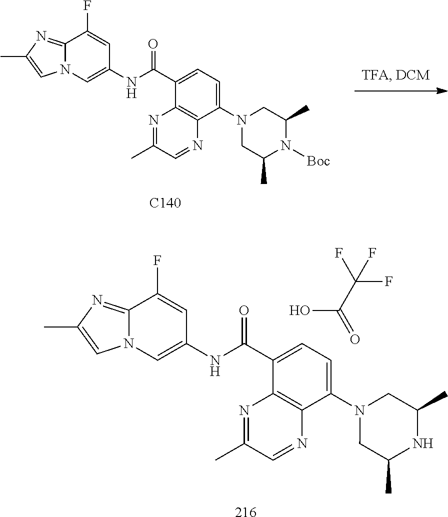

To a stirred solution of tert-butyl (2R,6S)-4-[8-({8-fluoro-2-methylimidazo[1,2-a]pyridin-6-yl}carbamoyl)-2-methylquinoxalin-5-yl]-2,6-dimethylpiperazine-1-carboxylate (40 mg, 0.073 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 12, Gradient 5) to afford 8-((3R,5S)-3,5-dimethylpiperazin-1-yl)-N-{8-fluoro-2-methylimidazo[1,2-a]pyridin-6-yl}-3-methylquinoxaline-5-carboxamide; bis(trifluoroacetic acid) (4.8 mg, 10%) as a solid. LCMS (ES, m/z): 448 [M+H]⁺. ¹H NMR (300 MHz, Methanol-d₄) δ 9.37 (d, J=1.5 Hz, 1H), 8.94 (s, 1H), 8.74 (d, J=8.4 Hz, 1H), 7.95 (s, 1H), 7.63 (d, J=11.5 Hz, 1H), 7.41 (d, J=8.5 Hz, 1H), 4.39 (d, J=12.8 Hz, 2H), 3.76 (d, J=7.3 Hz, 2H), 3.06 (d, J=12.4 Hz, 2H), 2.98 (s, 3H), 2.53 (d, J=0.9 Hz, 3H), 1.45 (d, J=6.6 Hz, 6H).

### Example 113: Synthesis of Compound 217

### Synthesis of Intermediate C141

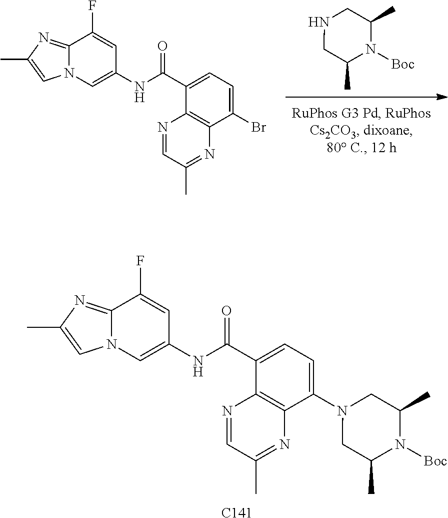 [SMILES: Cc1cnc2c(C(=O)Nc3cc(F)c4nc(C)cn4c3)ccc(Br)c2n1 ; C[C@@H]1CNC[C@H](C)N1C(=O)OC(C)(C)C ; Cc1cnc2c(C(=O)Nc3cc(F)c4nc(C)cn4c3)ccc(N3C[C@@H](C)N(C(=O)OC(C)(C)C)[C@@H](C)C3)c2n1 ; "C141"]

To a stirred mixture of 8-bromo-N-{8-fluoro-2-methylimidazo[1,2-a]pyridin-6-yl}-2-methylquinoxaline-5-carboxamide (50 mg, 0.121 mmol, 1 equiv) and tert-butyl (2R,6S)-2,6-dimethylpiperazine-1-carboxylate (31.04 mg, 0.145 mmol, 1.2 equiv) in dioxane (1 mL) was added Cs₂CO₃ (117.98 mg, 0.363 mmol, 3 equiv), RuPhos (11.27 mg, 0.024 mmol, 0.2 equiv), and RuPhos Palladacycle Gen.3 (10.10 mg, 0.012 mmol, 0.1 equiv) at room temperature under nitrogen atmosphere. The resulting mixture was stirred for 12 h at 80° C. under nitrogen atmosphere, then concentrated under reduced pressure to give a residue. The residue was purified by silica gel column chromatography, eluted with CH₂Cl₂/MeOH (10:1) to afford tert-butyl (2R,6S)-4-[8-({8-fluoro-2-methylimidazo[1,2-a]pyridin-6-yl}carbamoyl)-3-methylquinoxalin-5-yl]-2,6-dimethylpiperazine-1-carboxylate (50 mg, 76%) as a solid. LCMS (ES, m/z): 548 [M+H]

### Synthesis of Compound 217

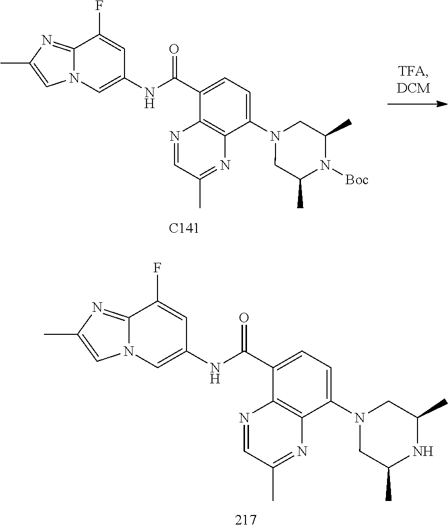

To a stirred solution of tert-butyl (2R,6S)-4-[8-({8-fluoro-2-methylimidazo[1,2-a]pyridin-6-yl}carbamoyl)-3-methylquinoxalin-5-yl]-2,6-dimethylpiperazine-1-carboxylate (50 mg, 0.091 mmol, 1 equiv) in DCM (1 mL) was added TFA (1 mL) at room temperature. The resulting mixture was stirred for 2 h at room temperature, then concentrated under reduced pressure to give a residue. The residue was purified by Prep-HPLC (Condition 11, Gradient 4) to afford 8-((3R,5S)-3, 5-dimethylpiperazin-1-yl)-N-{8-fluoro-2-methylimidazo[1,2-a]pyridin-6-yl}-2-methylquinoxaline-5-carboxamide (12.1 mg, 30%) as a solid. LCMS (ES, m/z): 448 [M+H]⁺. ¹H NMR (400 MHz, Methanol-d₄) δ 9.10 (d, J=1.6 Hz, 1H), 8.91 (s, 1H), 8.56 (d, J=8.5 Hz, 1H), 7.68 (d, J=2.9 Hz, 1H), 7.31-7.22 (m, 2H), 4.15 (d, J=11.7 Hz, 2H), 3.29 (d, J=2.7 Hz, 2H), 2.82 (s, 3H), 2.65 (t, J=11.3 Hz, 2H), 2.43 (s, 3H), 1.23 (d, J=6.4 Hz, 6H).

### Example 114: Synthesis of Compound 218

### Synthesis of Intermediate C142

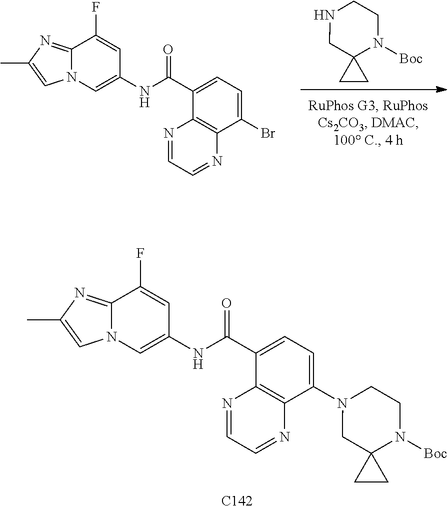 [SMILES: Cc1cn2cc(NC(=O)c3ccc(Br)c4nccnc34)cc(F)c2n1 ; CC(C)(C)OC(=O)N1CCNCC12CC2 ; Cc1cn2cc(NC(=O)c3ccc(N4CCN(C(=O)OC(C)(C)C)C5(CC5)C4)c4nccnc34)cc(F)c2n1 ; "C142"]

To a mixture of 8-bromo-N-{8-fluoro-2-methylimidazo[1,2-a]pyridin-6-yl}quinoxaline-5-carboxamide (120 mg, 0.300 mmol, 1 equiv) and tert-butyl 4,7-diazaspiro[2.5]octane-4-carboxylate (76.39 mg, 0.360 mmol, 1.2 equiv) in DMAC (2.5 mL) was added Cs₂CO₃ (293.08 mg, 0.900 mmol, 3 equiv), and RuPhos Palladacycle Gen.3 (25.08 mg, 0.030 mmol, 0.1 equiv).

After stirring for 4 h at 100° C. under a nitrogen atmosphere, the resulting mixture was concentrated under reduced pressure to give a residue. The residue was purified by silica gel column chromatography, eluted with (PE/THF=50%) to afford tert-butyl 7-[8-({8-fluoro-2-methylimidazo[1,2-a]pyridin-6-yl}carbamoyl)quinoxalin-5-yl]-4,7-diazaspiro[2.5]octane-4-carboxylate (60 mg, 38%) as a solid. LCMS (ES, m/z): 532 [M+H]⁺.

### Synthesis of Compound 248

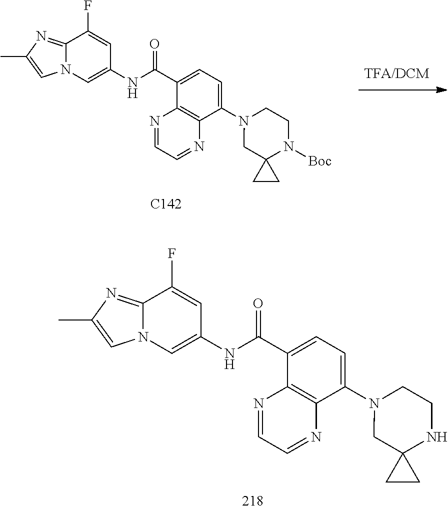 [SMILES: Cc1cn2cc(NC(=O)c3ccc(N4CCN(C(=O)OC(C)(C)C)C5(CC5)C4)c4nccnc34)cc(F)c2n1 ; "C142" ; Cc1cn2cc(NC(=O)c3ccc(N4CCNC5(CC5)C4)c4nccnc34)cc(F)c2n1 ; "218"]

A mixture of tert-butyl 7-[8-({8-fluoroimidazo[1,2-a]pyridin-6-yl}carbamoyl)quinoxalin-5-yl]-4,7-diazaspiro[2.5]octane-4-carboxylate (60 mg, 0.116 mmol, 1 equiv), DCM (3 mL) and TFA (1 mL) was stirred for 1 h at 25° C. The resulting mixture was concentrated under reduced pressure to give a residue. The residue was purified by Prep-HPLC (Condition 13, Gradient 1) to afford 8-{4,7-diazaspiro[2.5]octan-7-yl}-N-{8-fluoro-2-methylimidazo[1,2-a]pyridin-6-yl}quinoxaline-5-carboxamide (37.2 mg, 74%) as a solid. LCMS (ES, m/z): 432 [M+H]⁺. ¹H NMR (300 MHz, DMSO-d₆) δ 12.39 (s, 1H), 9.31-9.24 (m, 1H), 9.16-9.09 (m, 1H), 9.06-8.99 (m, 1H), 8.52 (d, J=8.6 Hz, 1H), 7.96-7.89 (m, 1H), 7.61-7.40 (m, 1H), 7.34 (d, J=8.6 Hz, 1H), 3.64 (s, 2H), 3.54 (s, 2H), 3.23 (s, 2H), 2.36 (s, 3H), 0.77 (s, 4H).

### Example 115: Synthesis of Compound 219

### Synthesis of Compound 219

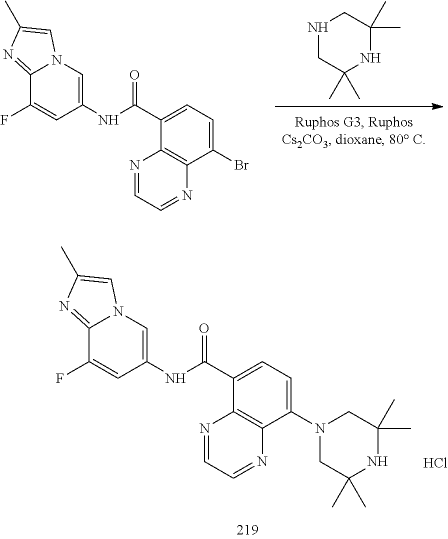

A mixture of 8-bromo-N-{8-fluoro-2-methylimidazo[1,2-a] pyridin-6-yl}quinoxaline-5-carboxamide (80 mg, 0.200 mmol, 1 equiv), 2,2,6,6-tetramethylpiperazine (42.65 mg, 0.300 mmol, 1.5 equiv), RuPhos (4.66 mg, 0.010 mmol, 0.05 equiv), RuPhos Palladacycle Gen.3 (8.36 mg, 0.010 mmol, 0.05 equiv), and Cs₂CO₃ (195.39 mg, 0.600 mmol, 3 equiv) in DMA (3 mL) was stirred for 2 h at 100° C. under N₂ atmosphere, then concentrated under reduced pressure to give a residue. The residue was purified by Prep-HPLC (Condition 14, Gradient 1) to afford N-{8-fluoro-2-methylimidazo[1,2-a]pyridin-6-yl}-8-(3,3,5,5-tetramethylpiperazin-1-yl)quinoxaline-5-carboxamide (5 mg, 99%) as a solid. LCMS (ES, m/z): 462 [M+H]⁺. ¹H NMR (300 MHz, DMSO-d6) δ 9.53 (s, 1H), 9.10 (s, 1H), 9.03 (s, 1H), 8.53 (d, J=8.3 Hz, 1H), 8.16 (d, J=12.5 Hz, 2H), 7.45 (d, J=8.5 Hz, 1H), 3.55 (s, 4H), 2.47 (s, 3H), 1.55 (s, 12H).

### Example 116: Synthesis of Compound 220

### Synthesis of Intermediate C143

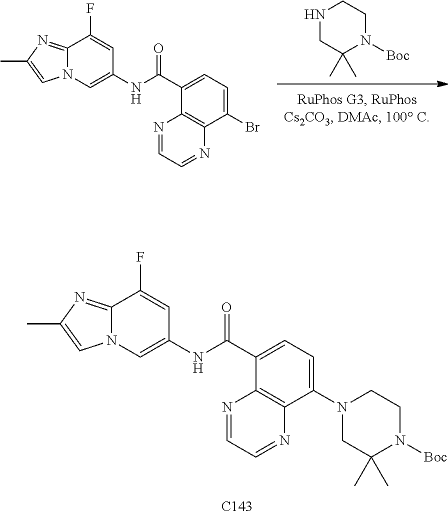 [SMILES: Cc1cn2cc(NC(=O)c3ccc(Br)c4nccnc34)cc(F)c2n1 ; CC(C)(C)OC(=O)N1CCNCC1(C)C ; Cc1cn2cc(NC(=O)c3ccc(N4CCN(C(=O)OC(C)(C)C)C(C)(C)C4)c4nccnc34)cc(F)c2n1 ; "C143"]

To a solution of 8-bromo-N-{8-fluoro-2-methylimidazo[1,2-a]pyridin-6-yl}quinoxaline-5-carboxamide (90 mg, 0.225 mmol, 1 equiv) and tert-butyl 4,7-diazaspiro[2.5]octane-4-carboxylate (76.39 mg, 0.360 mmol, 1.2 equiv) in DMAC (3 mL) was added Cs₂CO₃ (219.81 mg, 0.675 mmol, 3 equiv) and RuPhos Palladacycle Gen.3 (18.81 mg, 0.023 mmol, 0.1 equiv). After stirring for 4 h at 100° C. under a nitrogen atmosphere, the resulting mixture was concentrated under reduced pressure to give a residue. The residue was purified by silica gel column chromatography, eluted with (PE/THF=50%) to afford tert-butyl 7-[8-({8-fluoro-2-methylimidazo[1,2-a]pyridin-6-yl}carbamoyl)quinoxalin-5-yl]-4,7-diazaspiro[2.5]octane-4-carboxylate (60 mg, 38%) as a solid. LCMS (ES, m/z): 534 [M+H]⁺.

### Synthesis of Compound 220

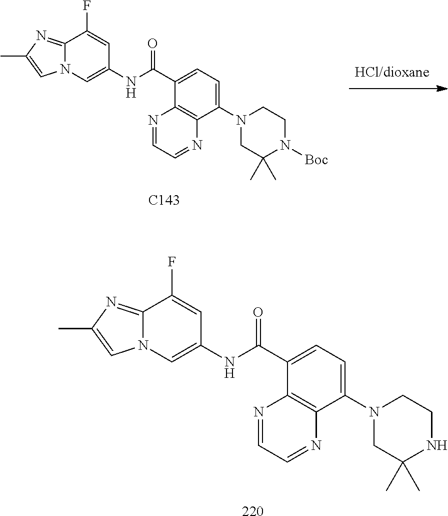 [SMILES: Cc1cn2cc(NC(=O)c3ccc(N4CCN(C(=O)OC(C)(C)C)C(C)(C)C4)c4nccnc34)cc(F)c2n1 ; "C143" ; Cc1cn2cc(NC(=O)c3ccc(N4CCNC(C)(C)C4)c4nccnc34)cc(F)c2n1 ; "220"]

A mixture of tert-butyl 4-[8-({8-fluoroimidazo[1,2-a]pyridin-6-yl}carbamoyl)quinoxalin-5-yl]-2,2-dimethylpiperazine-1-carboxylate (40 mg, 0.077 mmol, 1 equiv), DCM (3 mL), and TFA (1 mL) was stirred for 1 h at 25° C. The resulting mixture was concentrated under reduced pressure to give a residue. The residue was purified by Prep-HPLC (Condition 14, Gradient 2) to afford 8-(3,3-dimethylpiperazin-1-yl)-N-{8-fluoro-2-methylimidazo[1,2-a]pyridin-6-yl}quinoxaline-5-carboxamide (20 mg, 60%) as a solid. LCMS (ES, m/z): 434 [M+H]⁺. ¹H NMR (300 MHz, DMSO-d₆) δ 12.45 (s, 1H), 9.31-9.25 (m, 1H), 9.14-9.08 (m, 1H), 9.05-8.99 (m, 1H), 8.51 (, 1H), 7.96-7.89 (m, 1H), 7.51-7.41 (m, 1H), 7.34-7.26 (m, 1H), 3.48-3.39 (m, 2H), 3.38-3.30 (m, 2H), 3.02 (s, 2H), 2.36 (s, 3H), 1.93 (s, 1H), 1.17 (s, 6H).

### Example 117: Synthesis of Compound 221

### Synthesis of Intermediate C144

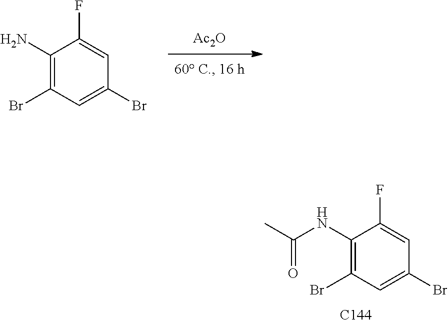

A mixture of 2,4-dibromo-6-fluoroaniline (2.6 g, 9.669 mmol, 1 equiv) in acetic anhydride (26 mL) was stirred for 16 h at 60° C. A precipitate formed that was collected by filtration and washed with water (2×30 mL). The solid was dissolved in DCM (50 mL), dried over anhydrous Na₂SO₄, and filtered. After filtration, the filtrate was concentrated under reduced pressure to afford N-(2,4-dibromo-6-fluorophenyl)acetamide (1.88 g, 63%) as a solid. LCMS (ES, m/z):310 [M+H]⁺.

### Synthesis of Intermediate C145

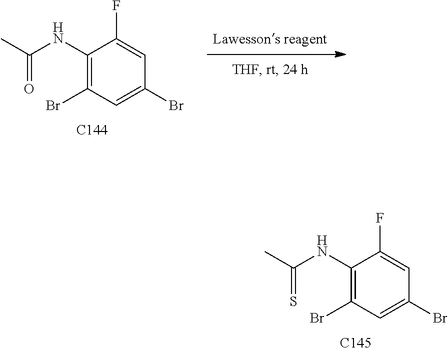

To a stirred mixture of N-(2,4-dibromo-6-fluorophenyl)acetamide (1.8 g, 5.789 mmol, 1.0 equiv) in THF (36 mL) was added bis(4-methoxyphenyl)-1,3,2I⁵,4I⁵-dithiadiphosphetane-2,4-dithione (2.81 g, 6.947 mmol, 1.2 equiv) at room temperature. The resulting mixture was stirred for 24 h, then diluted with water (50 mL). A precipitate formed that was collected by filtration and washed with water (3×20 mL). The solid was dissolved in DCM (50 mL), dried over anhydrous Na₂SO₄, and filtered. After filtration, the filtrate was concentrated under reduced pressure to afford N-(2,4-dibromo-6-fluorophenyl)ethanethioamide (1.7 g, 90%) as a solid. LCMS (ES, m/z):326 [M+H]⁺.

### Synthesis of Intermediate C146

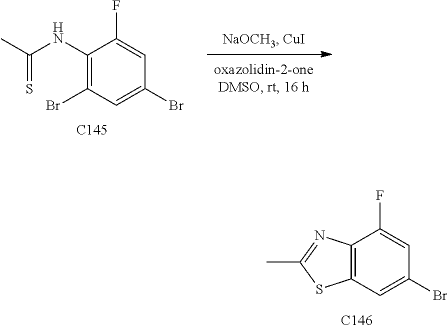

To a stirred solution of N-(2,4-dibromo-6-fluorophenyl)ethanethioamide (1.4 g, 4.281 mmol, 1.0 equiv) in DMSO (18 mL) was added NaOMe (0.69 g, 12.843 mmol, 3 equiv), 1,3-oxazolidin-2-one (0.07 g, 0.856 mmol, 0.2 equiv), and CuI (0.08 g, 0.428 mmol, 0.1 equiv) at room temperature under nitrogen atmosphere. The resulting mixture was stirred for 16 h at room temperature under nitrogen atmosphere, then diluted with water (50 mL) and extracted with ethyl acetate (3×50 mL). The organic layers were combined, washed with water (3×50 mL), dried over anhydrous Na₂SO₄, and filtered. After filtration, the filtrate was concentrated under reduced pressure to give a residue. The residue was purified by silica gel column chromatography, eluted with PE/DCM (1:1) to afford 6-bromo-4-fluoro-2-methyl-1,3-benzothiazole (350 mg, 33%) as a solid. LCMS (ES, m/z):246 [M+H]⁺.

### Synthesis of Intermediate C147

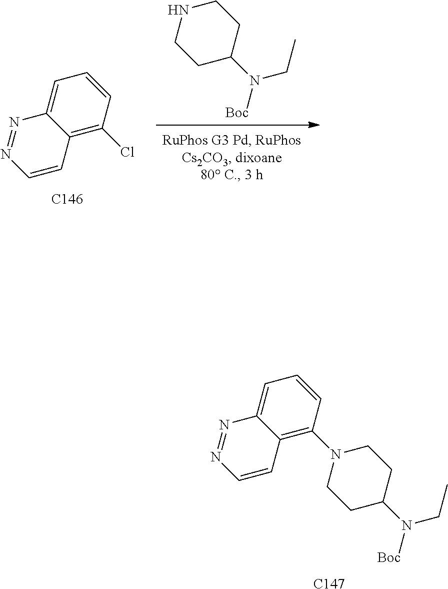 [SMILES: Clc1cccc2nnccc12 ; CCN(C(=O)OC(C)(C)C)C1CCNCC1 ; "C146" ; CCN(C(=O)OC(C)(C)C)C1CCN(c2cccc3nnccc23)CC1 ; "C147"]

To a stirred mixture of 5-chlorocinnoline (350.0 mg, 2.126 mmol, 1.0 equiv) and tert-butyl N-ethyl-N-(piperidin-4-yl)carbamate (582.7 mg, 2.551 mmol, 1.2 equiv) in dioxane (7 mL) was added Cs₂CO₃ (2.08 g, 6.378 mmol, 3 equiv), RuPhos (198.5 mg, 0.425 mmol, 0.2 equiv), and RuPhos Palladacycle Gen.3 (177.9 mg, 0.213 mmol, 0.1 equiv) at room temperature under nitrogen atmosphere. The resulting mixture was stirred for 3 h at 80° C. under nitrogen atmosphere, then cooled to room temperature. The resulting mixture was diluted with water (15 mL) and extracted with ethyl acetate (3×15 mL). The organic layers were combined, washed with water (3×15 mL), dried over anhydrous Na₂SO₄, and filtered. The filtrate was concentrated under reduced pressure to give a residue. The residue was purified by silica gel column chromatography, eluted with PE/EA (1:1) to afford tert-butyl N-[1-(cinnolin-5-yl)piperidin-4-yl]-N-ethylcarbamate (500 mg, 66%) as a solid. LCMS (ES, m/z):357 [M+H]⁺.

### Synthesis of Intermediate C148

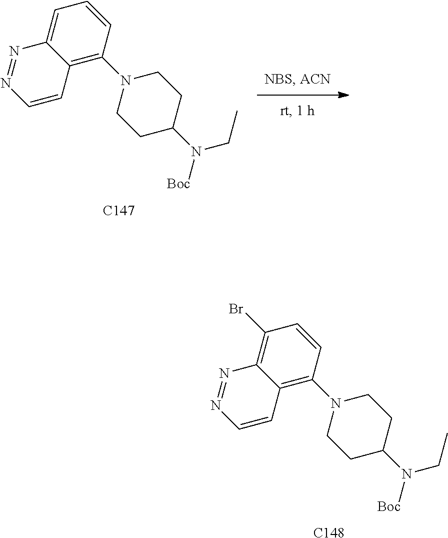 [SMILES: CCN(C(=O)OC(C)(C)C)C1CCN(c2cccc3nnccc23)CC1 ; "C147" ; CCN(C(=O)OC(C)(C)C)C1CCN(c2ccc(Br)c3nnccc23)CC1 ; "C148"]

To a stirred solution of tert-butyl N-[1-(cinnolin-5-yl)piperidin-4-yl]-N-ethylcarbamate (500.0 mg, 1.403 mmol, 1.0 equiv) in acetonitrile (10 mL) was added NBS (249.6 mg, 1.403 mmol, 1.0 equiv) at room temperature. The resulting mixture was stirred for 1 h at room temperature, then diluted with water (20 mL) and extracted with ethyl acetate (3×20 mL). The organic layers were combined, washed with water (2×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-[1-(8-bromocinnolin-5-yl)piperidin-4-yl]-N-ethylcarbamate (600 mg, 98%) as a solid. LCMS (ES, m/z):435 [M+H]⁺.

### Synthesis of Intermediate C149

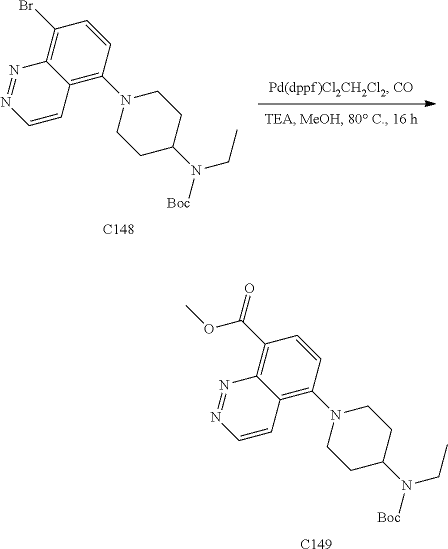 [SMILES: CCN(C(=O)OC(C)(C)C)C1CCN(c2ccc(Br)c3nnccc23)CC1 ; "C148" ; CCN(C(=O)OC(C)(C)C)C1CCN(c2ccc(C(=O)OC)c3nnccc23)CC1 ; "C149"]

To a solution of tert-butyl N-[1-(8-bromocinnolin-5-yl)piperidin-4-yl]-N-ethylcarbamate (600 mg, 1.378 mmol, 1 equiv) in methanol (25 mL) was added TEA (418.4 mg, 4.134 mmol, 3 equiv) and Pd(dppf)Cl₂CH₂Cl₂ (112.3 mg, 0.138 mmol, 0.1 equiv) in a pressure tank. The reaction mixture was purged with nitrogen for 2 min, then was pressurized to 1 MPa with carbon monoxide at 80° C. for 16 h. The reaction mixture was cooled to room temperature, then filtered. The filtrate was concentrated under reduced pressure to give a residue. The residue was purified by silica gel column chromatography, eluted with EA to afford methyl 5-{4-[(tert-butoxycarbonyl)(ethyl)amino]piperidin-1-yl}cinnoline-8-carboxylate (500 mg, 88%) as a solid. LCMS (ES, m/z):415 [M+H]⁺.

### Synthesis of Intermediate C50

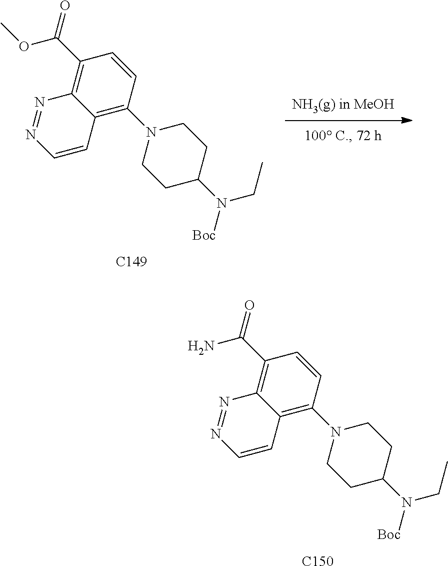 [SMILES: CCN(C(=O)OC(C)(C)C)C1CCN(c2ccc(C(=O)OC)c3nnccc23)CC1 ; "C149" ; CCN(C(=O)OC(C)(C)C)C1CCN(c2ccc(C(N)=O)c3nnccc23)CC1 ; "C150"]

A mixture of methyl 5-{4-[(tert-butoxycarbonyl)(ethyl)amino]piperidin-1-yl}cinnoline-8-carboxylate (500 mg, 1.206 mmol, 1 equiv) and NH₃(g) in methanol (40 mL) was stirred for 72 h at 100° C. The resulting mixture was cooled to room temperature, then concentrated under reduced pressure to give a residue. The residue was purified by silica gel column chromatography, eluted with EA to afford tert-butyl N-[1-(8-carbamoylcinnolin-5-yl)piperidin-4-yl]-N-ethylcarbamate (400 mg, 83%) as a solid. LCMS (ES, m/z):400 [M+H]⁺.

### Synthesis of Intermediate C151

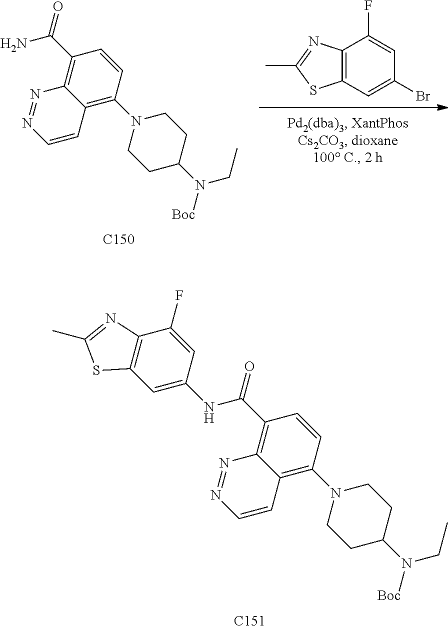 [SMILES: CCN(C(=O)OC(C)(C)C)C1CCN(c2ccc(C(N)=O)c3nnccc23)CC1 ; Cc1nc2c(F)cc(Br)cc2s1 ; "C150" ; CCN(C(=O)OC(C)(C)C)C1CCN(c2ccc(C(=O)Nc3cc(F)c4nc(C)sc4c3)c3nnccc23)CC1 ; "C151"]

To a stirred mixture of tert-butyl N-[1-(8-carbamoylcinnolin-5-yl)piperidin-4-yl]-N-ethylcarbamate (170 mg, 0.426 mmol, 1 equiv) and 6-bromo-4-fluoro-2-methyl-1,3-benzothiazole (104.7 mg, 0.426 mmol, 1.0 equiv) in dioxane (4.25 mL) was added Cs₂CO₃ (415.9 mg, 1.278 mmol, 3 equiv), XantPhos (49.3 mg, 0.085 mmol, 0.2 equiv), and Pd₂(dba)₃ (39.0 mg, 0.043 mmol, 0.1 equiv) at room temperature under nitrogen atmosphere. The resulting mixture was stirred for 2 h at 100° C. under nitrogen atmosphere, then cooled to room temperature, diluted with water (15 mL), and extracted with ethyl acetate (3×15 mL). The organic layers were combined, washed with water (3×15 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-ethyl-N-(1-{8-[(4-fluoro-2-methyl-1,3-benzothiazol-6-yl)carbamoyl]cinnolin-5-yl}piperidin-4-yl)carbamate (180 mg, 75%) as a solid. LCMS (ES, m/z):565 [M+H]⁺.

### Synthesis of Compound 221

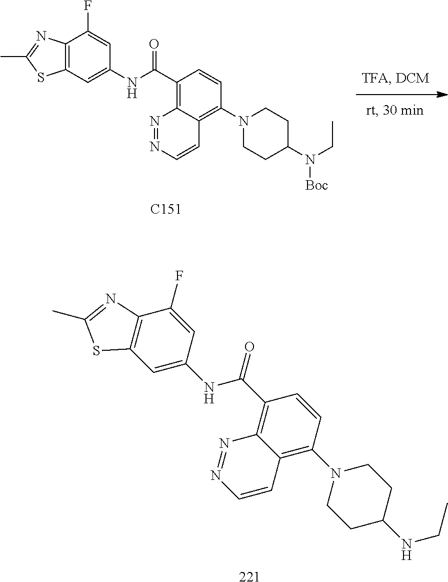

To a stirred solution of tert-butyl N-ethyl-N-(1-{8-[(4-fluoro-2-methyl-1,3-benzothiazol-6-yl)carbamoyl]cinnolin-5-yl}piperidin-4-yl)carbamate (100 mg, 0.177 mmol, 1.0 equiv) in DCM (2.5 mL) was added TFA (0.5 mL) at room temperature. The resulting mixture was stirred for 30 min at room temperature, then concentrated under vacuum to give a residue. The residue was purified by Prep-HPLC (Condition 11, Gradient 4) to afford 5-[4-(ethylamino)piperidin-1-yl]-N-(4-fluoro-2-methyl-1,3-benzothiazol-6-yl)cinnoline-8-carboxamide (35.3 mg, 43%) as a solid. LCMS (ES, m/z):465 [M+H]⁺. ¹H NMR (400 MHz, DMSO-d₆) δ 12.51 (s, 1H), 9.51 (d, J=5.9 Hz, 1H), 8.51 (d, J=8.0 Hz, 1H), 8.37-8.27 (m, 2H), 7.84 (dd, J=12.7, 1.9 Hz, 1H), 7.46 (d, J=8.1 Hz, 1H), 3.47 (d, J=12.2 Hz, 2H), 2.94 (t, J=11.4 Hz, 2H), 2.82 (s, 3H), 2.69-2.65 (m, 3H), 2.03 (d, J=12.5 Hz, 2H), 1.64 (q, J=10.9 Hz, 2H), 1.07 (t, J=7.1 Hz, 3H).

### Example 118: Synthesis of Compound 228

### Synthesis of Intermediate C152

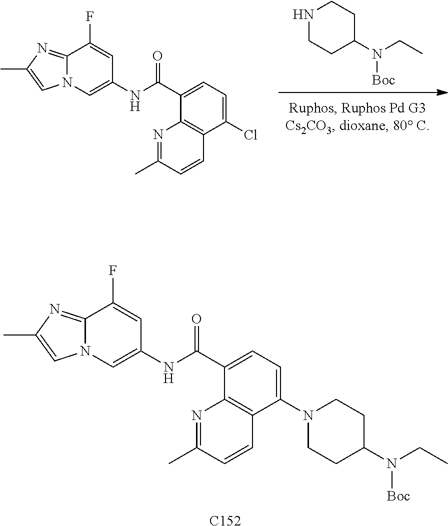 [SMILES: Cc1ccc2c(Cl)ccc(C(=O)Nc3cc(F)c4nc(C)cn4c3)c2n1 ; CCN(C(=O)OC(C)(C)C)C1CCNCC1 ; CCN(C(=O)OC(C)(C)C)C1CCN(c2ccc(C(=O)Nc3cc(F)c4nc(C)cn4c3)c3nc(C)ccc23)CC1 ; "C152"]

To a solution of 5-chloro-N-{8-fluoro-2-methylimidazo[1,2-a]pyridin-6-yl}-2-methylquinoline-8-carboxamide (130.0 mg, 0.352 mmol, 1.0 equiv) and tert-butyl N-ethyl-N-(piperidin-4-yl)carbamate (96.5 mg, 0.422 mmol, 1.2 equiv) in dioxane (4 mL) were added Cs₂CO₃ (288.0 mg, 0.880 mmol, 2.5 equiv) and Ruphos (32.9 mg, 0.070 mmol, 0.2 equiv), RuPhos Palladacycle Gen.3 (29.4 mg, 0.035 mmol, 0.1 equiv). After stirring for 2 hr at 80° C. under a nitrogen atmosphere, the resulting mixture was concentrated under reduced pressure. The residue was purified by silica gel column chromatography, eluted with CH₂Cl₂/MeOH (30:1) to afford tert-butyl N-ethyl-N-{1-[8-({8-fluoro-2-methylimidazo[1,2-a]pyridin-6-yl}carbamoyl)-2-methylquinolin-5-yl]piperidin-4-yl}carbamate (130 mg, 52%) as a solid. LCMS (ES, m/z): 561 [M+H]⁺

### Synthesis of Compound 228

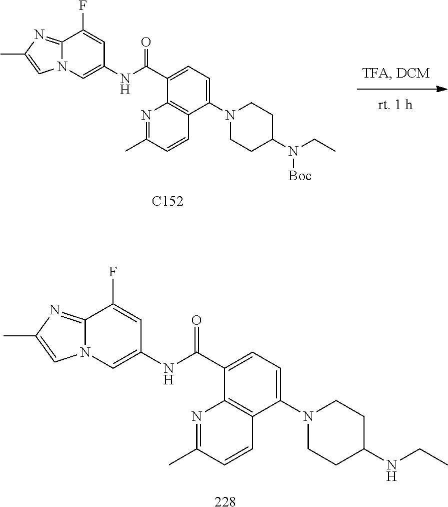

To a stirred solution of tert-butyl N-ethyl-N-{1-[8-({8-fluoro-2-methylimidazo[1,2-a]pyridin-6-yl} carbamoyl)-2-methylquinolin-5-yl]piperidin-4-yl}carbamate (90.0 mg, 0.161 mmol, 1.0 equiv) in DCM (2 mL) was added TFA (0.5 mL) dropwise at room temperature. The resulting mixture was stirred for 1 hr at room temperature. The resulting mixture was concentrated under reduced pressure. The crude product was purified by Prep-HPLC (Condition 15, Gradient 1) to afford 5-[4-(ethylamino)piperidin-1-yl]-N-{8-fluoro-2-methylimidazo[1,2-a]pyridin-6-yl}-2-methylquinoline-8-carboxamide (28.8 mg, 38%) as a solid. LCMS (ES, m/z): 461 [M+H]⁺ 1H NMR (400 MHz, DMSO-d₆) δ 13.78 (s, 1H), 9.27 (d, J=1.7 Hz, 1H), 8.58 (d, J=8.2 Hz, 1H), 8.49 (d, J=8.7 Hz, 1H), 7.93 (d, J=3.1 Hz, 1H), 7.60 (d, J=8.6 Hz, 1H), 7.33-7.23 (m, 2H), 3.41 (d, J=11.7 Hz, 2H), 2.95-2.85 (m, 5H), 2.67-2.60 (m, 3H), 2.36 (s, 3H), 2.01 (d, J=12.4 Hz, 2H), 1.62 (d, J=12.0 Hz, 2H), 1.06 (t, J=7.1 Hz, 3H).

### Example 119: Synthesis of Compound 232

### Synthesis of Intermediate C153

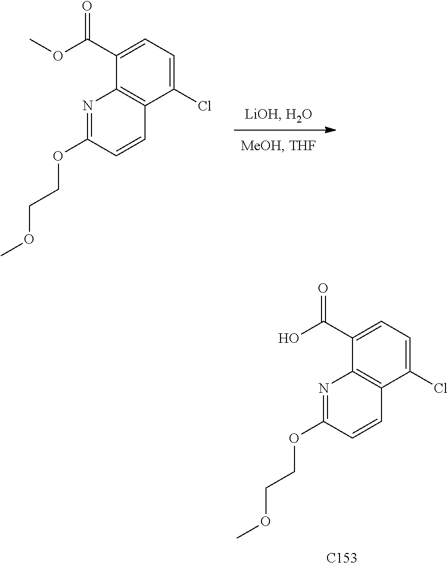 [SMILES: COCCOc1ccc2c(Cl)ccc(C(=O)OC)c2n1 ; COCCOc1ccc2c(Cl)ccc(C(=O)O)c2n1 ; "C153"]

A solution of methyl 5-chloro-2-(2-methoxyethoxy)quinoline-8-carboxylate (100 mg, 0.338 mmol, 1 equiv) and LiOH (40.49 mg, 1.690 mmol, 5 equiv) in THE (2 mL), H₂O (2 mL) and MeOH (0.2 mL) was stirred for 1 h at room temperature. The mixture was acidified to pH 3 with HCl (aq.). The precipitated solids were collected by filtration and washed with water (3×10 mL). This resulted in 5-chloro-2-(2-methoxyethoxy)quinoline-8-carboxylic acid (70 mg, 73%) as a solid. LCMS (ES, m/z):282 [M+H]⁺

### Synthesis of Intermediate C154

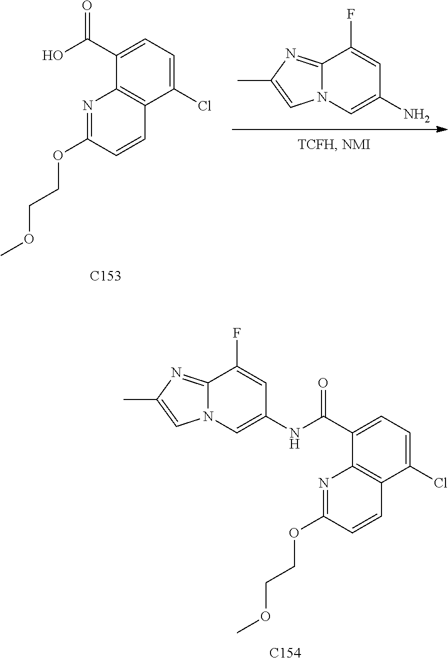

To a stirred solution of 5-chloro-2-(2-methoxyethoxy)quinoline-8-carboxylic acid (100 mg, 0.355 mmol, 1 equiv) and 8-fluoro-2-methylimidazo[1,2-a]pyridin-6-amine (70 mg, 0.426 mmol, 1.2 equiv) in CH₃CN (5 mL) were added NMI (116 mg, 1.420 mmol, 4 equiv) and TCFH (129 mg, 0.461 mmol, 1.3 equiv) at room temperature. The resulting mixture was stirred for additional 0.5 h at room temperature. The precipitated solids were collected by filtration and washed with CH₃CN (3×10 mL) to afford 5-chloro-N-{8-fluoro-2-methylimidazo[1,2-a]pyridin-6-yl}-2-(2-methoxyethoxy)quinoline-8-carboxamide (100 mg, 65%) as a solid. LCMS (ES, m/z): 429 [M+H]

### Synthesis of Intermediate C155

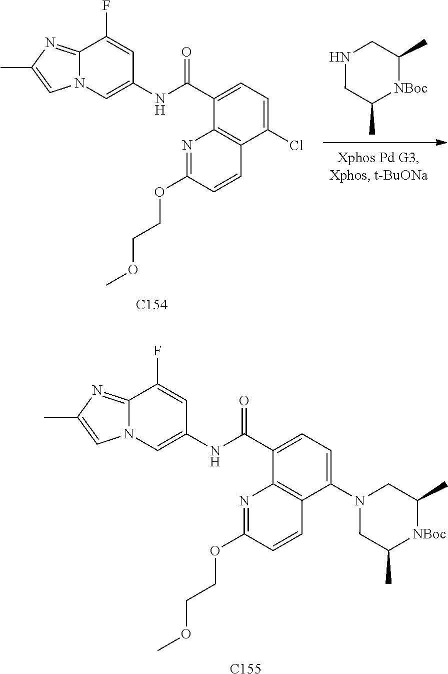

To a stirred solution of 5-chloro-N-{8-fluoro-2-methylimidazo[1,2-a]pyridin-6-yl}-2-(2-methoxyethoxy)quinoline-8-carboxamide (100 mg, 0.233 mmol, 1 equiv), t-BuONa (67 mg, 0.699 mmol, 3 equiv) and tert-butyl (cis)-2,6-dimethylpiperazine-1-carboxylate (60 mg, 0.280 mmol, 1.2 equiv) in dioxane (5 mL) were added XPhos Pd G3 (20 mg, 0.023 mmol, 0.1 equiv) and XPhos (22 mg, 0.047 mmol, 0.2 equiv) at room temperature under nitrogen atmosphere. The resulting mixture was stirred for 3 hr at 100° C. under nitrogen atmosphere. The resulting mixture was concentrated under reduced pressure. The residue was purified by silica gel column chromatography, eluted with PE/EA (1:4) to afford tert-butyl (2R,6S)-4-[8-({8-fluoro-2-methylimidazo[1,2-a]pyridin-6-yl}carbamoyl)-2-(2-methoxyethoxy)quinolin-5-yl]-2,6-dimethylpiperazine-1-carboxylate (60 mg, 42%) as a solid. LCMS (ES, m/z): 607 [M+H]⁺

### Synthesis of Compound 232

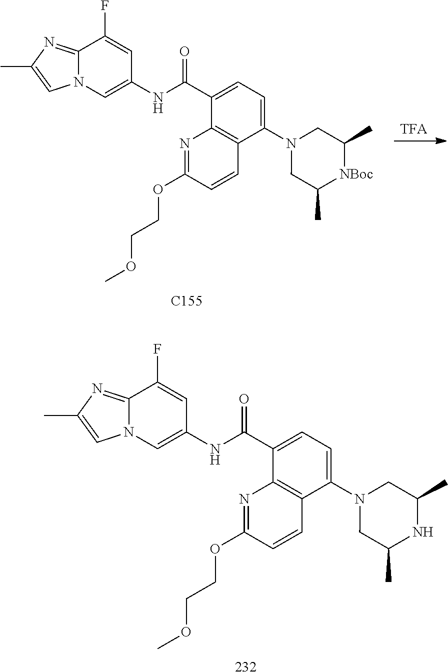

A solution of tert-butyl (cis)-4-[8-({8-fluoro-2-methylimidazo[1,2-a]pyridin-6-yl}carbamoyl)-2-(2-methoxyethoxy)quinolin-5-yl]-2,6-dimethylpiperazine-1-carboxylate (80 mg, 0.132 mmol, 1 equiv) in TFA (5 mL) and DCM (5 mL) was stirred for 3 h at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 3, Gradient 1) to afford 5-[(3R,5S)-3,5-dimethylpiperazin-1-yl]-N-{8-fluoro-2-methylimidazo[1,2-a]pyridin-6-yl}-2-(2-methoxyethoxy)quinoline-8-carboxamide (8 mg, 11%) as a solid. LCMS (ES, m/z): 507 [M+H]^{+ 1}H NMR (300 MHz, DMSO-d6) δ 11.91 (s, 1H), 9.19 (d, J=1.6 Hz, 1H), 8.47 (d, J=9.2 Hz, 1H), 8.40 (d, J=8.2 Hz, 1H), 7.92 (s, 1H), 7.26 (d, J=12.6 Hz, 1H), 7.22-7.13 (m, 2H), 4.68 (t, J=4.9 Hz, 2H), 3.78 (t, J=4.9 Hz, 2H), 3.23 (d, J=11.2 Hz, 6H), 3.14 (s, 2H), 2.39 (d, J=17.7 Hz, 5H), 1.04 (d, J=6.2 Hz, 6H).

### Example 120: Synthesis of Compound 233

### Synthesis of Intermediate C156

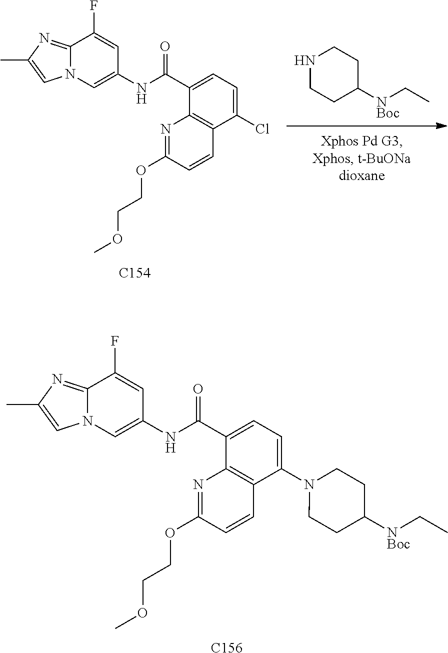 [SMILES: COCCOc1ccc2c(Cl)ccc(C(=O)Nc3cc(F)c4nc(C)cn4c3)c2n1 ; CCN(C(=O)OC(C)(C)C)C1CCNCC1 ; "C154" ; CCN(C(=O)OC(C)(C)C)C1CCN(c2ccc(C(=O)Nc3cc(F)c4nc(C)cn4c3)c3nc(OCCOC)ccc23)CC1 ; "C156"]

To a stirred solution of 5-chloro-N-{8-fluoro-2-methylimidazo[1,2-a]pyridin-6-yl}-2-(2-methoxyethoxy)quinoline-8-carboxamide (100 mg, 0.233 mmol, 1 equiv), sodium 2-methylpropan-2-olate (67 mg, 0.699 mmol, 3 equiv) and tert-butyl N-ethyl-N-(piperidin-4-yl)carbamate (64 mg, 0.280 mmol, 1.2 equiv) in dioxane (5 mL) were added XPhos Pd G3 (20 mg, 0.023 mmol, 0.1 equiv) and XPhos (22 mg, 0.047 mmol, 0.2 equiv) at room temperature under nitrogen atmosphere. The resulting mixture was stirred for 3 hr at 100° C. under nitrogen atmosphere. The resulting mixture was concentrated under reduced pressure. The residue was purified by silica gel column chromatography, eluted with PE/EA (1:4) to afford tert-butyl N-ethyl-N-{1-[8-({8-fluoro-2-methylimidazo[1,2-a]pyridin-6-yl}carbamoyl)-2-(2-methoxyethoxy)quinolin-5-yl]piperidin-4-yl}carbamate (100 mg, 69%) as a solid. LCMS (ES, m/z): 621 [M+H]⁺

### Synthesis of Compound 233

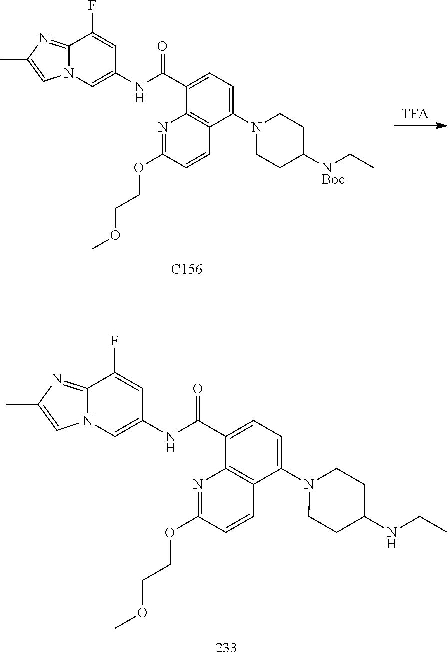

A solution of tert-butyl N-ethyl-N-{1-[8-({8-fluoro-2-methylimidazo[1,2-a]pyridin-6-yl}carbamoyl)-2-(2-methoxyethoxy)quinolin-5-yl]piperidin-4-yl}carbamate (80 mg, 0.129 mmol, 1 equiv) in TFA (5 mL) and DCM (5 mL) was stirred for 3 hr at room temperature. The resulting mixture was concentrated under reduced pressure. The residue was basified to pH 8 with 7M NH₃(g) in methanol. The resulting mixture was concentrated under vacuum. The residue was purified by reverse flash chromatography (Condition 3, Gradient 2) to afford 5-[4-(ethylamino)piperidin-1-yl]-N-{8-fluoro-2-methylimidazo[1,2-a]pyridin-6-yl}-2-(2-methoxyethoxy)quinoline-8-carboxamide (9 mg, 13%) as a solid. LCMS (ES, m/z): 521 [M+H]^{+ 1}H NMR (300 MHz, DMSO-d₆) δ 11.89 (s, 1H), 9.22-9.16 (m, 1H), 8.41 (t, J=8.4 Hz, 2H), 7.92 (s, 1H), 7.31-7.13 (m, 3H), 4.68 (d, J=5.3 Hz, 2H), 3.78 (t, J=4.8 Hz, 2H), 3.28 (t, J=4.8 Hz, 6H) 2.87 (t, J=11.3 Hz, 2H), 2.68 (q, J=7.7, 7.1 Hz, 3H), 2.36 (s, 3H), 2.02 (d, J=12.3 Hz, 2H), 1.63 (d, J=11.2 Hz, 2H), 1.08 (t, J=7.1 Hz, 3H).

### Example 121: Synthesis of Compound 229

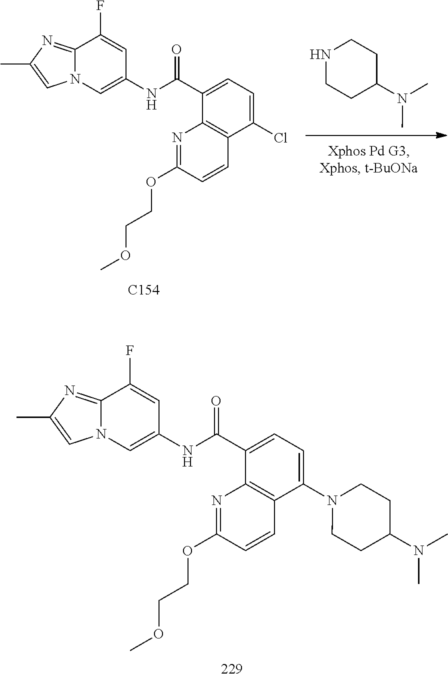

To a stirred solution of 5-chloro-N-{8-fluoro-2-methylimidazo[1,2-a]pyridin-6-yl}-2-(2-methoxyethoxy)quinoline-8-carboxamide (100 mg, 0.233 mmol, 1 equiv), sodium 2-methylpropan-2-olate (67 mg, 0.699 mmol, 3 equiv) and N,N-dimethylpiperidin-4-amine (36 mg, 0.280 mmol, 1.2 equiv) in dioxane (5 mL) were added XPhos Pd G3 (194 mg, 0.023 mmol, 0.1 equiv) and XPhos (22 mg, 0.047 mmol, 0.2 equiv) at room temperature under nitrogen atmosphere.

The resulting mixture was stirred for 3 hr at 100° C. under nitrogen atmosphere. The resulting mixture was concentrated under reduced pressure. The residue was purified by reverse flash chromatography (Condition 4, Gradient 1) to afford bis(2,2-difluoropropanoic acid); 5-[4-(dimethylamino)piperidin-1-yl]-N-{8-fluoro-2-methylimidazo[1,2-a]pyridin-6-yl}-2-(2-methoxyethoxy)quinoline-8-carboxamide (16 mg, 9%) as a solid. LCMS (ES, m/z): 521 [M+H]^{+ 1}H NMR (300 MHz, DMSO-d₆) δ 11.99 (s, 1H), 10.15 (s, 1H), 9.44 (d, J=1.5 Hz, 1H), 8.46 (d, J=9.2 Hz, 1H), 8.38 (d, J=8.2 Hz, 1H), 8.16 (dd, J=2.7, 1.2 Hz, 1H), 7.65 (dd, J=12.1, 1.5 Hz, 1H), 7.22 (t, J=8.4 Hz, 2H), 4.72-4.63 (m, 2H), 3.83-3.73 (m, 2H), 3.51 (d, J=12.1 Hz, 4H), 2.96-2.82 (m, 8H), 2.48-2.42 (m, 3H), 2.17 (d, J=11.6 Hz, 2H), 2.04 (s, 2H), 1.98 (d, J=11.8 Hz, 2H).

### Example 122: Synthesis of Compound 226

### Synthesis of Intermediate C157

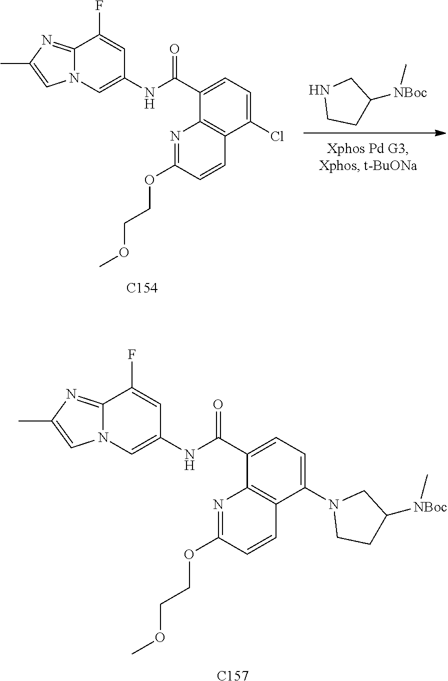

To a stirred solution of 5-chloro-N-{8-fluoro-2-methylimidazo[1,2-a]pyridin-6-yl}-2-(2-methoxyethoxy)quinoline-8-carboxamide (100 mg, 0.233 mmol, 1 equiv), sodium 2-methylpropan-2-olate (67 mg, 0.699 mmol, 3 equiv) and tert-butyl N-methyl-N-(pyrrolidin-3-yl)carbamate (56 mg, 0.280 mmol, 1.2 equiv) in dioxane (5 mL) were added XPhos Pd G3 (20 mg, 0.023 mmol, 0.1 equiv) and XPhos (22 mg, 0.047 mmol, 0.2 equiv) at room temperature under nitrogen atmosphere. The resulting mixture was stirred for 3 hr at 100° C. under nitrogen atmosphere. The resulting mixture was concentrated under reduced pressure. The residue was purified by silica gel column chromatography, eluted with PE/EA (1:4) to afford tert-butyl N-{1-[8-({8-fluoro-2-methylimidazo[1,2-a]pyridin-6-yl}carbamoyl)-2-(2-methoxyethoxy)quinolin-5-yl]pyrrolidin-3-yl}-N-methylcarbamate (90 mg, 65%) as a solid. LCMS (ES, m/z): 593 [M+H]⁺

### Synthesis of Compound 226

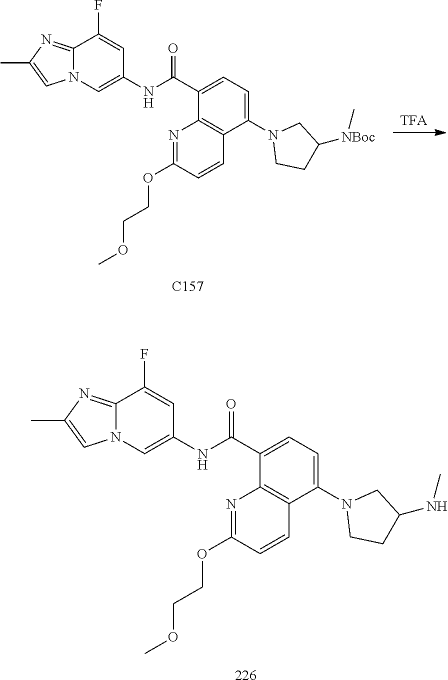

A solution of tert-butyl N-{1-[8-({8-fluoro-2-methylimidazo[1,2-a]pyridin-6-yl}carbamoyl)-2-(2-methoxyethoxy)quinolin-5-yl]pyrrolidin-3-yl}-N-methylcarbamate (80 mg, 0.135 mmol, 1 equiv) in TFA (5 mL) and DCM (5 mL) was stirred for 3 hr at room temperature. The resulting mixture was concentrated under reduced pressure. The residue was basified to pH 8 with 7M NH₃(g) in MeOH. The resulting mixture was concentrated under vacuum. The residue was purified by reverse flash chromatography (Condition 3, Gradient 2) to afford N-{8-fluoro-2-methylimidazo[1,2-a]pyridin-6-yl}-2-(2-methoxyethoxy)-5-[3-(methylamino)pyrrolidin-1-yl]quinoline-8-carboxamide (10 mg, 15%) as a solid. LCMS (ES, m/z): 493 [M+H]^{+ 1}H NMR (300 MHz, DMSO-d₆) δ 12.10 (s, 1H), 9.16 (s, 1H), 8.68 (d, J=9.3 Hz, 1H), 8.36 (d, J=8.7 Hz, 1H), 7.90 (s, 1H), 7.25 (d, J=12.5 Hz, 1H), 7.00 (d, J=9.2 Hz, 1H), 6.82 (d, J=8.8 Hz, 1H), 4.68 (s, 2H), 3.80 (d, J=6.1 Hz, 8H), 3.54 (s, 3H), 2.34 (d, J=8.1 Hz, 6H), 2.10 (s, 1H), 1.90 (s, 1H).

### Example 123: Synthesis of Compound 230

### Synthesis of Intermediate C158

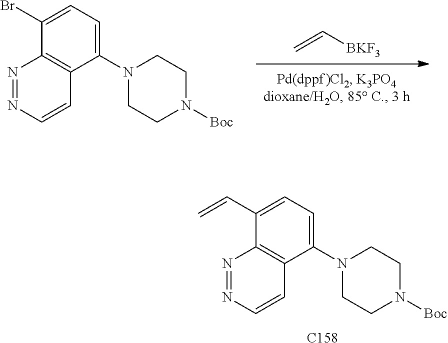

To a solution of tert-butyl 4-(8-bromocinnolin-5-yl)piperazine-1-carboxylate (300 mg, 0.76 mmol, 1.0 equiv) and ethenyltrifluoropotassio-lambda5-borane (122.6 mg, 0.91 mmol, 1.2 equiv) in 1,4-dioxane (3 mL) and H₂O (0.3 mL) were added K₃PO₄ (485.7 mg, 2.29 mmol, 3.0 equiv) and Pd(dppf)Cl₂ (55.8 mg, 0.07 mmol, 0.1 equiv). After stirring for 3 hr at 85° C. under a nitrogen atmosphere, the resulting mixture was concentrated under reduced pressure. The residue was purified by silica gel column chromatography, eluted with PE/EA (2:1) to afford tert-butyl 4-(8-ethenylcinnolin-5-yl)piperazine-1-carboxylate (152 mg, 58%) as a solid. LCMS (ES, m/z): 341 [M+H]⁺

### Synthesis of Intermediate C159

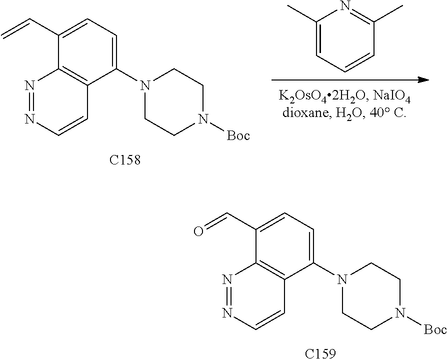

To a stirred mixture of tert-butyl 4-(8-ethenylcinnolin-5-yl)piperazine-1-carboxylate (152 mg, 0.44 mmol, 1.0 equiv) and lutidine (95.6 mg, 0.89 mmol, 2.0 equiv) in dioxane (3.7 mL) and H₂O (1.5 mL) were added NaIO₄ (382.0 mg, 1.78 mmol, 4.0 equiv) and K₂OsO₄·2H₂O (16.4 mg, 0.04 mmol, 0.1 equiv). The resulting mixture was stirred for 3 hr at 40° C. The resulting mixture was diluted with water. The resulting mixture was extracted with ethyl acetate (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with EA to afford tert-butyl 4-(8-formylcinnolin-5-yl)piperazine-1-carboxylate (89 mg, 58%) as a solid. LCMS (ES, m/z): 343 [M+H]⁺

### Synthesis of Intermediate C160

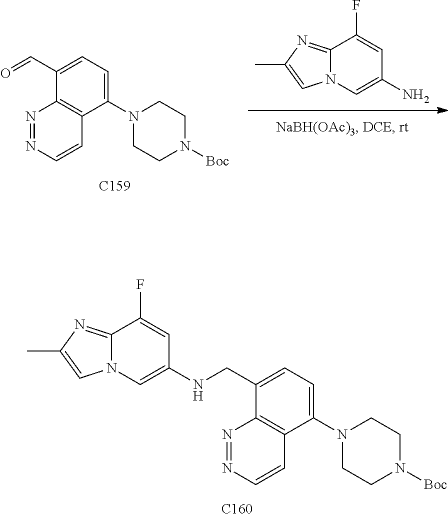 [SMILES: CC(C)(C)OC(=O)N1CCN(c2ccc(C=O)c3nnccc23)CC1 ; Cc1cn2cc(N)cc(F)c2n1 ; "C159" ; Cc1cn2cc(NCc3ccc(N4CCN(C(=O)OC(C)(C)C)CC4)c4ccnnc34)cc(F)c2n1 ; "C160"]

To a stirred mixture of tert-butyl 4-(8-formylcinnolin-5-yl)piperazine-1-carboxylate (89 mg, 0.26 mmol, 1.0 equiv) and 8-fluoro-2-methylimidazo[1,2-a]pyridin-6-amine (47.2 mg, 0.28 mmol, 1.1 equiv) in DCE (1 mL) was added NaBH(OAc)₃ (110.1 mg, 0.52 mmol, 2 equiv) in portions at 0° C. under nitrogen atmosphere. The resulting mixture was stirred for 3 h at room temperature. The resulting mixture was diluted with water. The resulting mixture was extracted with ethyl acetate (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with CH₂Cl₂/MeOH (30:1) to afford tert-butyl 4-{8-[({8-fluoro-2-methylimidazo[1,2-a]pyridin-6-yl}amino)methyl]cinnolin-5-yl}piperazine-1-carboxylate (61 mg, 47%) as a solid. LCMS (ES, m/z): 492 [M+H]⁺

### Synthesis of Compound 230

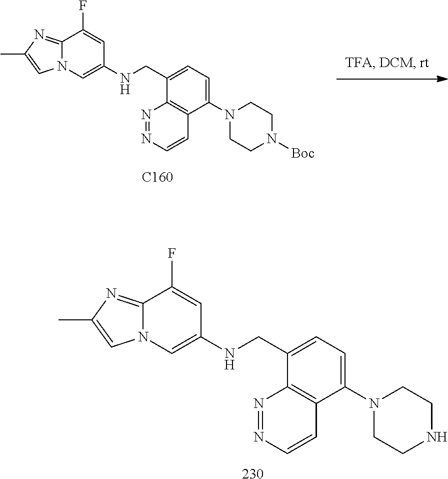

To a stirred solution of tert-butyl 4-{8-[({8-fluoro-2-methylimidazo[1,2-a]pyridin-6-yl}amino)methyl]cinnolin-5-yl}piperazine-1-carboxylate (61 mg, 0.124 mmol, 1 equiv) in DCM (0.6 mL) was added TFA (0.2 mL) at room temperature. The resulting mixture was stirred for 1 h at room temperature. The resulting mixture was concentrated under vacuum. The crude product was purified by Prep-HPLC (Condition 11, Gradient 5) to afford 8-fluoro-2-methyl-N-{[5-(piperazin-1-yl)cinnolin-8-yl]methyl}imidazo[1,2-a]pyridin-6-amine (22 mg, 43%) as a solid.

LCMS (ES, m/z): 392 [M+H]^{+ 1}H NMR (400 MHz, DMSO-d₆) δ 9.42 (d, J=5.9 Hz, 1H), 8.22 (d, J=6.0 Hz, 1H), 7.79 (d, J=7.7 Hz, 1H), 7.52 (d, J=3.1 Hz, 1H), 7.50 (d, J=1.8 Hz, 1H), 7.32 (d, J=7.7 Hz, 1H), 6.84 (dd, J=13.1, 1.8 Hz, 1H), 6.12 (t, J=6.0 Hz, 1H), 4.97 (d, J=5.9 Hz, 2H), 2.96 (d, J=2.8 Hz, 8H), 2.24 (s, 3H).

### Example 124: Synthesis of Compound 231

### Synthesis of Intermediate C161

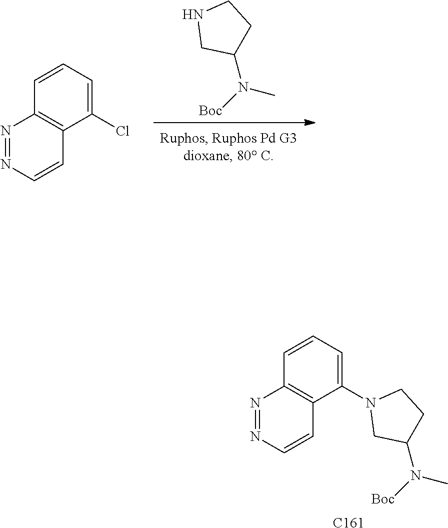 [SMILES: Clc1cccc2nnccc12 ; CN(C(=O)OC(C)(C)C)C1CCNC1 ; CN(C(=O)OC(C)(C)C)C1CCN(c2cccc3nnccc23)C1 ; "C161"]

To a solution of 5-chlorocinnoline (300.0 mg, 1.823 mmol, 1.0 equiv) and tert-butyl N-methyl-N-(pyrrolidin-3-yl)carbamate (438.0 mg, 2.188 mmol, 1.2 equiv) in dioxane (5 mL) were added Cs₂CO₃ (1489.2 mg, 4.558 mmol, 2.5 equiv) and Ruphos (170.1 mg, 0.365 mmol, 0.2 equiv), RuPhos Palladacycle Gen.3 (152.4 mg, 0.182 mmol, 0.1 equiv). After stirring for 3 hr at 80° C. under a nitrogen atmosphere, the resulting mixture was concentrated under reduced pressure. The residue was purified by silica gel column chromatography, eluted with PE/EA (1:1) to afford tert-butyl N-[1-(cinnolin-5-yl) pyrrolidin-3-yl]-N-methylcarbamate (320 mg, 45%) as a solid. LCMS (ES, m/z): 329 [M+H]⁺

### Synthesis of Intermediate C162

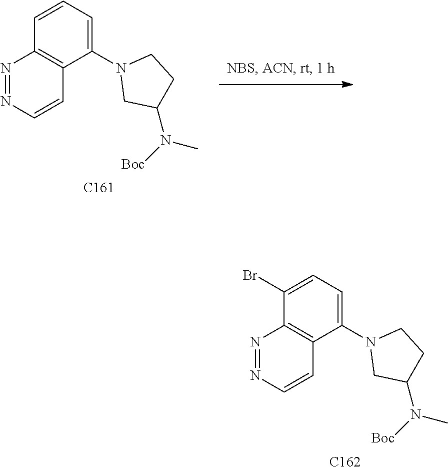 [SMILES: CN(C(=O)OC(C)(C)C)C1CCN(c2cccc3nnccc23)C1 ; "C161" ; CN(C(=O)OC(C)(C)C)C1CCN(c2ccc(Br)c3nnccc23)C1 ; "C162"]

Into a 40 mL vial were added tert-butyl N-[1-(cinnolin-5-yl) pyrrolidin-3-yl]-N-methylcarbamate (320.0 mg, 0.974 mmol, 1.0 equiv), ACN (10 mL) and NBS (173.4 mg, 0.974 mmol, 1.0 equiv) at room temperature. The resulting mixture was stirred for 1 hr at room temperature. The resulting mixture was diluted with deionized water (50 mL). The resulting mixture was extracted with ethyl acetate (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (1:2) to afford tert-butyl N-[1-(8-bromocinnolin-5-yl)pyrrolidin-3-yl]-N-methylcarbamate (380 mg, 86%) as a solid. LCMS (ES, m/z): 407 [M+H]*Synthesis of Intermediate C163

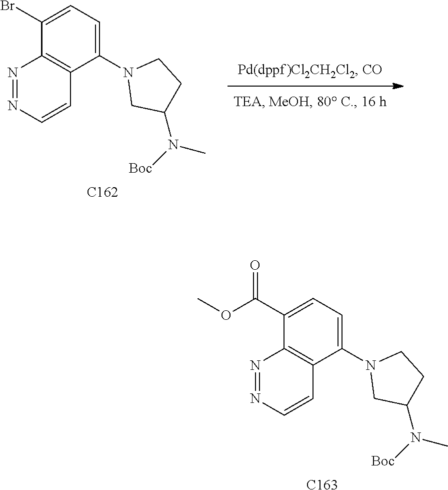

To a solution of tert-butyl N-[1-(8-bromocinnolin-5-yl) pyrrolidin-3-yl]-N-methylcarbamate (320.0 mg, 0.786 mmol, 1.0 equiv) in 20 mL MeOH was added Pd(dppf)Cl₂ (64.0 mg, 0.079 mmol, 0.1 equiv) in a pressure tank. The mixture was purged with nitrogen for 2 min and then was pressurized to 2Mpa with carbon monoxide at 80° C. for 16 hr. The reaction mixture was cooled to room temperature and filtered to remove insoluble solids. The resulting mixture was concentrated under reduced pressure. The residue was purified by silica gel column chromatography, eluted with CH₂Cl₂/MeOH (20:1) to afford methyl 5-{3-[(tert-butoxycarbonyl) (methyl)amino]pyrrolidin-1-yl}cinnoline-8-carboxylate (290 mg, 85%) as a solid. LCMS (ES, m/z): 387 [M+H]⁺

### Synthesis of Intermediate C164

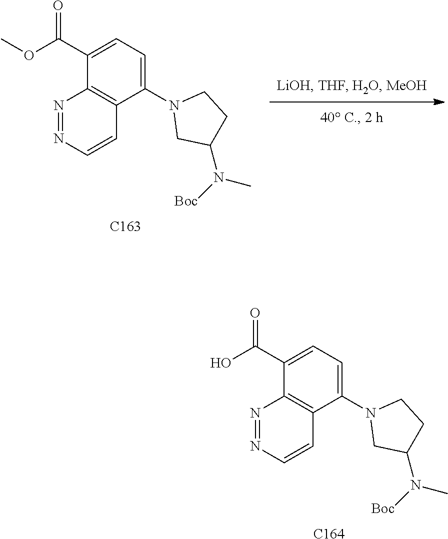

Into a 40 mL vial were added methyl 5-{3-[(tert-butoxycarbonyl)(methyl)amino]pyrrolidin-1-yl}cinnoline-8-carboxylate (290.0 mg, 0.750 mmol, 1.0 equiv), tetrahydrofuran (3 mL), methanol (3 mL), water (3 mL) and LiOH (179.7 mg, 7.500 mmol, 10.0 equiv) at room temperature. The resulting mixture was stirred for 2 hr at 40° C. The resulting mixture was diluted with deionized water (30 mL). The mixture was acidified to pH 6 with HCl (aq.). The resulting mixture was extracted with ethyl acetate (2×30 mL). The combined organic layers were washed with brine (1×30 mL), dried over anhydrous Na₂SO₄. After filtration, the filtrate was concentrated under reduced pressure. This resulted in 5-{3-[(tert-butoxycarbonyl) (methyl)amino]pyrrolidin-1-yl}cinnoline-8-carboxylic acid (260 mg, 86%) as a solid. LCMS (ES, m/z): 373 [M+H]⁺

### Synthesis of Intermediate C165

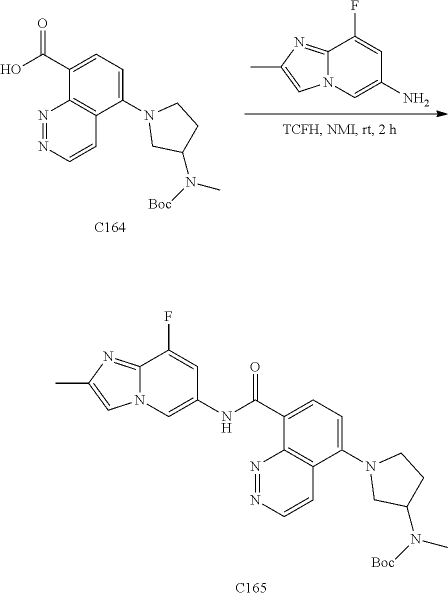

To a stirred solution of 5-{3-[(tert-butoxycarbonyl)(methyl)amino]pyrrolidin-1-yl}cinnoline-8-carboxylic acid (200.0 mg, 0.537 mmol, 1.0 equiv), NMI (176.3 mg, 2.148 mmol, 4.0 equiv) and 8-fluoro-2-methylimidazo[1,2-a]pyridin-6-amine (97.5 mg, 0.591 mmol, 1.1 equiv) in ACN (6 mL) was added TCFH (195.8 mg, 0.698 mmol, 1.3 equiv) in portions at room temperature. The resulting mixture was stirred for 1 hr at room temperature. The reaction was monitored by LCMS. The resulting mixture was diluted with water (30 mL). The resulting mixture was extracted with CH₂Cl₂ (2×30 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (20:1) to afford tert-butyl N-{1-[8-({8-fluoro-2-methylimidazo[1,2-a]pyridin-6-yl} carbamoyl)cinnolin-5-yl]pyrrolidin-3-yl}-N-methylcarbamate (260 mg, 83%) as a solid. LCMS (ES, m/z): 520 [M+H]⁺

### Synthesis of Compound 231

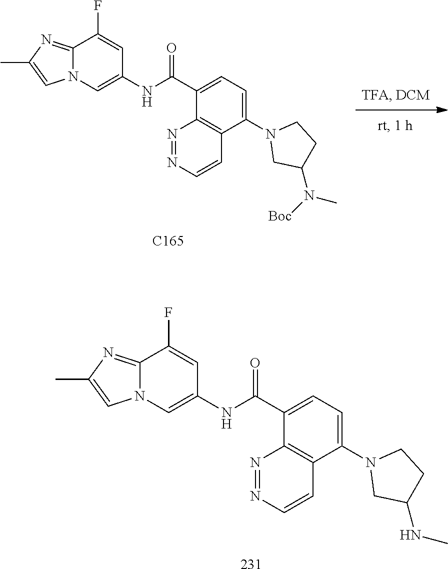

Into a 40 mL vial were added tert-butyl N-{1-[8-({8-fluoro-2-methylimidazo[1,2-a]pyridin-6-yl} carbamoyl)cinnolin-5-yl]pyrrolidin-3-yl}-N-methylcarbamate (200.0 mg, 0.385 mmol, 1.0 equiv), DCM (2 mL) and TFA (0.5 mL) at room temperature. The resulting mixture was stirred for 1 h at room temperature. The resulting mixture was concentrated under reduced pressure. The crude product was purified by Prep-HPLC (Condition 10, Gradient 2) to afford N-{8-fluoro-2-methylimidazo[1,2-a]pyridin-6-yl}-5-[3-(methylamino)pyrrolidin-1-yl] cinnoline-8-carboxamide (43 mg, 26%) as an solid. LCMS (ES, m/z): 420 [M+H]^{+ 1}H NMR (400 MHz, DMSO-d₆) δ 12.72 (s, 1H), 9.35 (d, J=6.1 Hz, 1H), 9.20 (d, J=1.6 Hz, 1H), 8.64 (d, J=6.1 Hz, 1H), 8.56 (d, J=8.6 Hz, 1H), 7.91 (d, J=3.0 Hz, 1H), 7.34 (dd, J=12.5, 1.7 Hz, 1H), 6.94 (d, J=8.8 Hz, 1H), 3.96-3.80 (m, 2H), 3.69 (q, J=8.4, 7.5 Hz, 1H), 3.51 (dd, J=10.3, 4.0 Hz, 1H), 2.50-2.49 (m, 1H), 2.36 (s, 3H), 2.32 (s, 3H), 2.12 (dt, J=12.6, 6.3 Hz, 1H), 1.91 (dt, J=11.8, 6.0 Hz, 1H).

### Example 125: Synthesis of Compound 235 and 236

### Synthesis of Compound 235

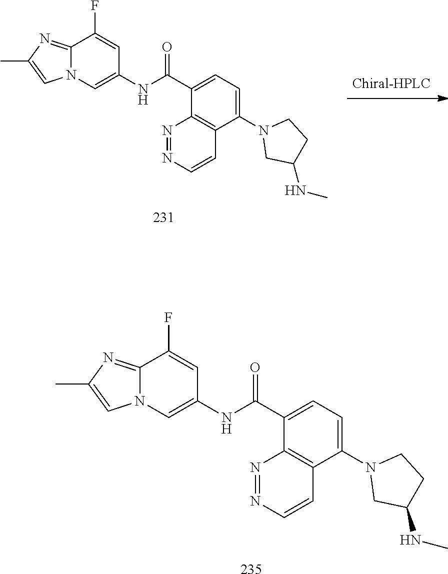

Compound 228 was separated by Prep-Chiral HPLC (Condition 3, Gradient 1) to afford (R)—N-(8-fluoro-2-methylimidazo[1,2-a]pyridin-6-yl)-5-(3-(methylamino)pyrrolidin-1-yl)cinnoline-8-carboxamide (10 mg, 28%) as a solid. LCMS (ES, m/z): 420 [M+H]⁺ retention time: 4.371 ¹H NMR (400 MHz, DMSO-d₆) δ 12.72 (s, 1H), 9.35 (d, J=6.1 Hz, 1H), 9.20 (d, J=1.6 Hz, 1H), 8.64 (d, J=6.1 Hz, 1H), 8.56 (d, J=8.6 Hz, 1H), 7.91 (d, J=3.0 Hz, 1H), 7.34 (dd, J=12.5, 1.7 Hz, 1H), 6.94 (d, J=8.8 Hz, 1H), 3.96-3.80 (m, 2H), 3.69 (q, J=8.4, 7.5 Hz, 1H), 3.51 (dd, J=10.3, 4.0 Hz, 1H), 2.50-2.49 (m, 1H), 2.36 (s, 3H), 2.32 (s, 3H), 2.12 (dt, J=12.6, 6.3 Hz, 1H), 1.91 (dt, J=11.8, 6.0 Hz, 1H).

### Synthesis of Compound 236

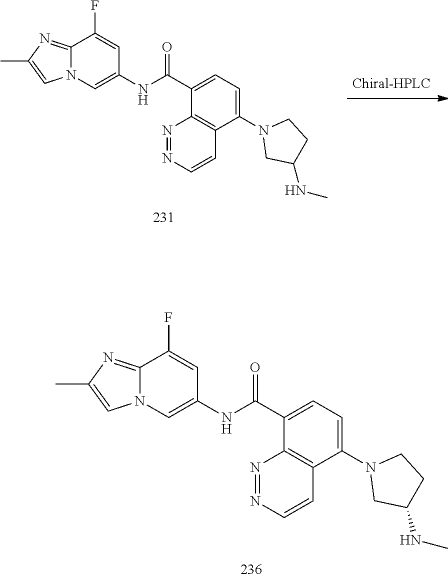

Compound 228 was separated by Prep-Chiral HPLC (Condition 3, Gradient 1) to yield (S)—N-(8-fluoro-2-methylimidazo[1,2-a]pyridin-6-yl)-5-(3-(methylamino)pyrrolidin-1-yl)cinnoline-8-carboxamide (10 mg, 28%) as a solid. LCMS (ES, m/z): 420 [M+H]⁺ retention time: 4.998 ¹H NMR (400 MHz, DMSO-d₆) δ 12.72 (s, 1H), 9.35 (d, J=6.1 Hz, 1H), 9.20 (d, J=1.6 Hz, 1H), 8.64 (d, J=6.1 Hz, 1H), 8.56 (d, J=8.6 Hz, 1H), 7.91 (d, J=3.0 Hz, 1H), 7.34 (dd, J=12.5, 1.7 Hz, 1H), 6.94 (d, J=8.8 Hz, 1H), 3.96-3.80 (m, 2H), 3.69 (q, J=8.4, 7.5 Hz, 1H), 3.51 (dd, J=10.3, 4.0 Hz, 1H), 2.50-2.49 (m, 1H), 2.36 (s, 3H), 2.32 (s, 3H), 2.12 (dt, J=12.6, 6.3 Hz, 1H), 1.91 (dt, J=11.8, 6.0 Hz, 1H).

### Example 126: Synthesis of Compound 242

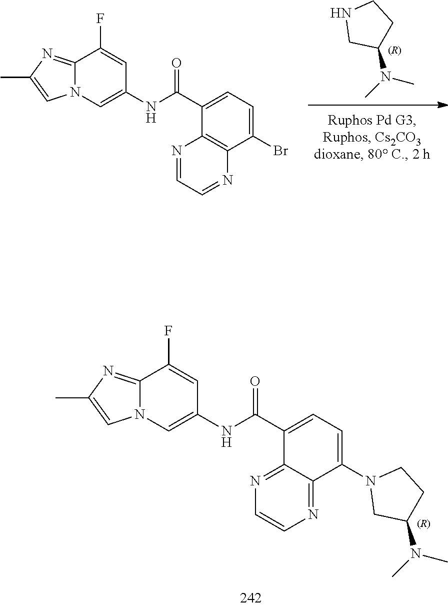

To a solution of 8-bromo-N-{8-fluoro-2-methylimidazo[1,2-a]pyridin-6-yl}quinoxaline-5-carboxamide (60 mg, 0.150 mmol, 1.0 equiv) and (3R)—N,N-dimethylpyrrolidin-3-amine (25.6 mg, 0.225 mmol, 1.5 equiv) in dioxane (2 mL) were added Cs₂CO₃ (98.0 mg, 0.300 mmol, 2 equiv) and Ruphos (13.9 mg, 0.030 mmol, 0.2 equiv), RuPhos Palladacycle Gen.3 (25.0 mg, 0.030 mmol, 0.2 equiv). After stirring for 2 h at 80° C. under nitrogen atmosphere, the resulting mixture was concentrated under reduced pressure. The crude product was purified by Prep-HPLC (Condition 10, Gradient 2) to afford 8-[(3R)-3-(dimethylamino)pyrrolidin-1-yl]-N-{8-fluoro-2-methylimidazo[1,2-a]pyridin-6-yl}quinoxaline-5-carboxamide (23.4 mg, 35%) as a solid. LCMS (ES, m/z): 434 [M+H]^{+ 1}H NMR (300 MHz, DMSO-d₆) δ 12.61 (s, 1H), 9.23 (d, J=1.6 Hz, 1H), 9.02 (d, J=1.8 Hz, 1H), 8.89 (d, J=1.8 Hz, 1H), 8.47 (d, J=8.9 Hz, 1H), 7.92-7.85 (m, 1H), 7.42 (dd, J=12.6, 1.7 Hz, 1H), 6.88 (d, J=9.0 Hz, 1H), 4.06-3.84 (m, 3H), 3.74-3.62 (m, 1H), 2.84-2.73 (m, 1H), 2.35 (s, 3H), 2.25-2.20 (m, 7H), 1.83 (p, J=11.1, 10.5 Hz, 1H).

### Example 127: Synthesis of Compound 240

### Synthesis of Intermediate C166

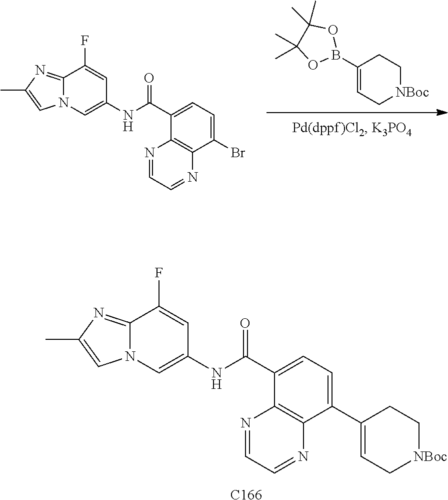 [SMILES: Cc1cn2cc(NC(=O)c3ccc(Br)c4nccnc34)cc(F)c2n1 ; CC(C)(C)OC(=O)N1CC=C(B2OC(C)(C)C(C)(C)O2)CC1 ; Cc1cn2cc(NC(=O)c3ccc(C4=CCN(C(=O)OC(C)(C)C)CC4)c4nccnc34)cc(F)c2n1 ; "C166"]

To a stirred solution of 8-bromo-N-{8-fluoro-2-methylimidazo[1,2-a]pyridin-6-yl}quinoxaline-5-carboxamide (150 mg, 0.375 mmol, 1 equiv) and tert-butyl 4-(4,4,5,5-tetramethyl-1,3,2-dioxaborolan-2-yl)-3,6-dihydro-2H-pyridine-1-carboxylate (128 mg, 0.413 mmol, 1.1 equiv) in dioxane (3 mL) and H₂O (0.6 mL) were added K₃PO₄ (239 mg, 1.125 mmol, 3 equiv) and Pd(dppf)Cl₂ (27 mg, 0.038 mmol, 0.1 equiv) at room temperature under nitrogen atmosphere. The resulting mixture was stirred for 3 hr at 80° C. The resulting mixture was concentrated under reduced pressure. The residue was purified by silica gel column chromatography, eluted with PE/EA (0:1) to afford tert-butyl 4-[8-({8-fluoro-2-methylimidazo[1,2-a]pyridin-6-yl}carbamoyl)quinoxalin-5-yl]-3,6-dihydro-2H-pyridine-1-carboxylate (100 mg, 53%) as a solid. LCMS (ES, m/z):503 [M+H]⁺

### Synthesis of Compound 240

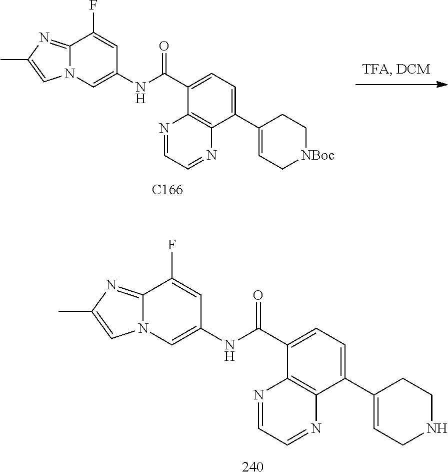

A solution of tert-butyl 4-[8-({8-fluoro-2-methylimidazo[1,2-a]pyridin-6-yl}carbamoyl)quinoxalin-5-yl]-3,6-dihydro-2H-pyridine-1-carboxylate (100 mg, 0.199 mmol, 1 equiv) and trifluoroacetic acid (2 mL) in DCM (3 mL) was stirred for 2 hr at room temperature. The resulting mixture was concentrated under vacuum. The residue was purified by reverse flash (Condition 3, Gradient 3) to afford N-{8-fluoro-2-methylimidazo[1,2-a]pyridin-6-yl}-8-(1,2,3,6-tetrahydropyridin-4-yl)quinoxaline-5-carboxamide (21 mg, 26%) as a solid. LCMS (ES, m/z): 402[M+H]^{+ 1}H NMR (300 MHz, DMSO-d6) δ 11.90 (s, 1H), 9.29 (d, J=1.6 Hz, 1H), 9.13 (q, J=1.9 Hz, 2H), 8.42 (d, J=7.6 Hz, 1H), 7.95 (d, J=3.1 Hz, 1H), 7.83 (d, J=7.6 Hz, 1H), 7.36 (dd, J=12.5, 1.7 Hz, 1H), 6.09 (s, 1H), 3.46 (q, J=2.9 Hz, 2H), 2.98 (t, J=5.5 Hz, 2H), 2.59 (s, 2H), 2.36 (s, 3H).

### Example 128: Synthesis of Compound 223

### Synthesis of Intermediate C168

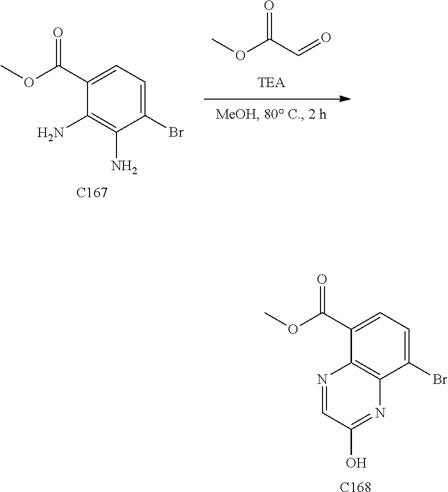

To a stirred solution of methyl 2,3-diamino-4-bromobenzoate (10 g, 40.804 mmol, 1 equiv), TEA (4.95 g, 48.965 mmol, 1.2 equiv) in EtOH (40 mL) was added methyl 2-oxoacetate (4.31 g, 48.965 mmol, 1.2 equiv) dropwise at room temperature. The resulting mixture was stirred for 2 h at 80° C. The resulting mixture was concentrated under reduced pressure. The residue was purified by reverse phase flash (Condition 3, Gradient 1) to afford methyl 8-bromo-2-hydroxyquinoxaline-5-carboxylate (1.5 g, 13%) as a solid. LCMS (ES, m/z): 283[M+H]⁺

### Synthesis of Intermediate C169

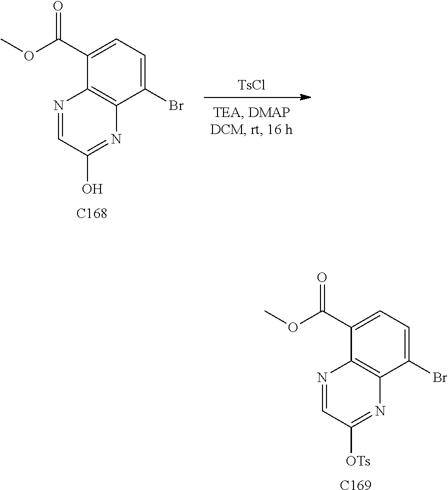 [SMILES: COC(=O)c1ccc(Br)c2nc(O)cnc12 ; "TsCl" ; "C168" ; COC(=O)c1ccc(Br)c2nc(OS(=O)(=O)c3ccc(C)cc3)cnc12 ; "C169"]

To a stirred mixture of methyl 8-bromo-2-hydroxyquinoxaline-5-carboxylate (1.5 g, 5.299 mmol, 1 equiv) and TEA (0.80 g, 7.949 mmol, 1.5 equiv) in DCM (15 mL) was added DMAP (0.13 g, 1.060 mmol, 0.2 equiv) and P-toluenesulfonyl chloride (1.21 g, 6.359 mmol, 1.2 equiv) in portions at 0° C. The resulting mixture was stirred for 16 h at room temperature. The resulting mixture was diluted with H₂O (10 mL) and extracted with DCM (2×30 mL). The combined organic layers were washed with brine (1×30 mL), dried over anhydrous Na₂SO₄. After filtration, the filtrate was concentrated under reduced pressure to afford methyl 8-bromo-2-[(4-methylbenzenesulfonyl)oxy]quinoxaline-5-carboxylate (1.48 g, 64%) as a solid. LCMS (ES, m/z): 437[M+H]⁺

### Synthesis of Intermediate C170

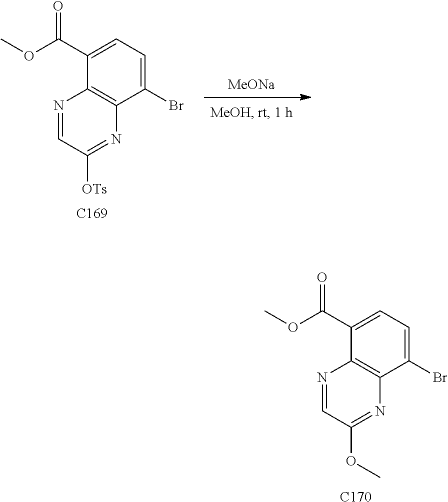 [SMILES: "MeONa" ; COC(=O)c1ccc(Br)c2nc(OS(=O)(=O)c3ccc(C)cc3)cnc12 ; "C169" ; COC(=O)c1ccc(Br)c2nc(OC)cnc12 ; "C170"]

To a stirred solution of methyl 8-bromo-2-[(4-methylbenzenesulfonyl)oxy]quinoxaline-5-carboxylate (1.48 g, 3.385 mmol, 1 equiv) in methanol (15 mL) was added MeONa (0.20 g, 3.724 mmol, 1.1 equiv) dropwise at room temperature. The resulting mixture was stirred for 2 h at room temperature. The resulting mixture was concentrated under reduced pressure. The residue was purified by silica gel column chromatography, eluted with PE/EA (4:1) to afford methyl 8-bromo-2-methoxyquinoxaline-5-carboxylate (850 mg, 85%) as a solid. LCMS (ES, m/z): 297[M+H]⁺

### Synthesis of Intermediate C171

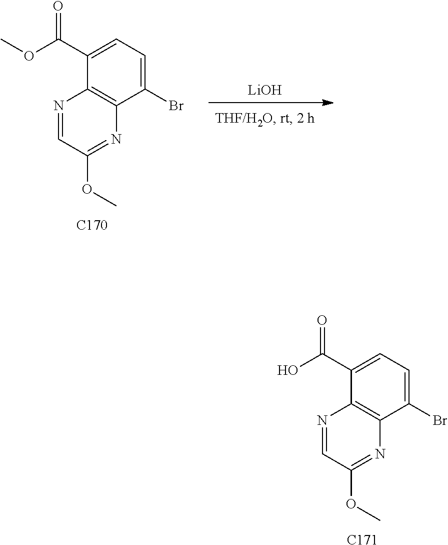 [SMILES: COC(=O)c1ccc(Br)c2nc(OC)cnc12 ; "LiOH" ; "C170" ; COc1cnc2c(C(=O)O)ccc(Br)c2n1 ; "C171"]

To a stirred solution of methyl 8-bromo-2-methoxyquinoxaline-5-carboxylate (850 mg, 2.861 mmol, 1 equiv) in THE (9 mL) and H₂O (3 mL) was added lithiumol (205.56 mg, 8.583 mmol, 3 equiv) at room temperature. The resulting mixture was stirred overnight at room temperature. The resulting mixture was concentrated under vacuum and diluted with H₂O (20 mL). The solution was acidified to pH 3 with citric acid. The precipitated solids were collected by filtration and dried under infrared light to afford 8-bromo-2-methoxyquinoxaline-5-carboxylic acid (730 mg, 90%) as a solid. LCMS (ES, m/z): 283[M+H]⁺

### Synthesis of Intermediate C172

To a stirred mixture of 8-bromo-2-methoxyquinoxaline-5-carboxylic acid (730 mg, 2.579 mmol, 1 equiv) and HATU (1176.65 mg, 3.095 mmol, 1.2 equiv) in DCM (15 mL) was added DIEA (1499.84 mg, 11.606 mmol, 4.5 equiv) and 8-fluoro-2-methylimidazo[1,2-a]pyridin-6-amine hydrochloride (623.95 mg, 3.095 mmol, 1.2 equiv) at room temperature. The resulting mixture was stirred overnight at room temperature. The resulting mixture was concentrated under vacuum, diluted with H₂O (5 mL) and stirred for 30 min at room temperature. The precipitated solids were collected by filtration and dried under infrared light to afford 8-bromo-N-{8-fluoro-2-methylimidazo[1,2-a]pyridin-6-yl}-2-methoxyquinoxaline-5-carboxamide (940 mg, 85%) as a solid. LCMS (ES, m/z): 430[M−H]⁻

### Synthesis of Intermediate C172

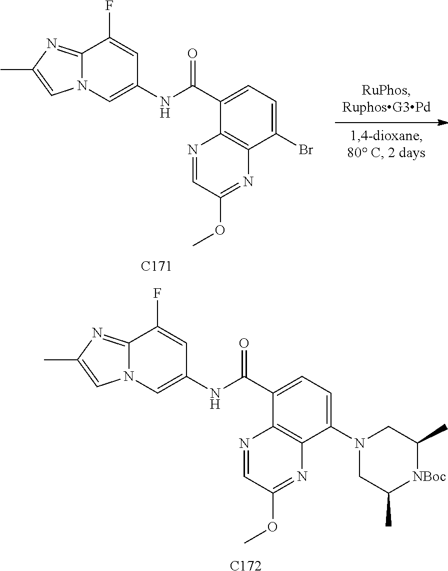

To a stirred mixture of 8-bromo-N-{8-fluoro-2-methylimidazo[1,2-a]pyridin-6-yl}-2-methoxyquinoxaline-5-carboxamide (120 mg, 0.279 mmol, 1 equiv) and tert-butyl 2,6-dimethylpiperazine-1-carboxylate (71.73 mg, 0.335 mmol, 1.2 equiv), Cs₂CO₃ (272.63 mg, 0.837 mmol, 3 equiv) in 1,4-dioxane (4 mL) was added Ruphos (13.02 mg, 0.028 mmol, 0.1 equiv) and RuPhos Palladacycle Gen.3 (23.33 mg, 0.028 mmol, 0.1 equiv) at room temperature under nitrogen atmosphere. The resulting mixture was stirred for 2 days at 80° C. under nitrogen atmosphere. The resulting mixture was concentrated under vacuum. The residue was purified by silica gel column chromatography, eluted with CH₂Cl₂/MeOH (50:1) to afford tert-butyl 4-[8-({8-fluoro-2-methylimidazo[1,2-a]pyridin-6-yl}carbamoyl)-3-methoxyquinoxalin-5-yl]-2,6-dimethylpiperazine-1-carboxylate (60 mg, 38%) as a solid. LCMS (ES, m/z): 564[M−H]⁻

### Synthesis of Compound 223

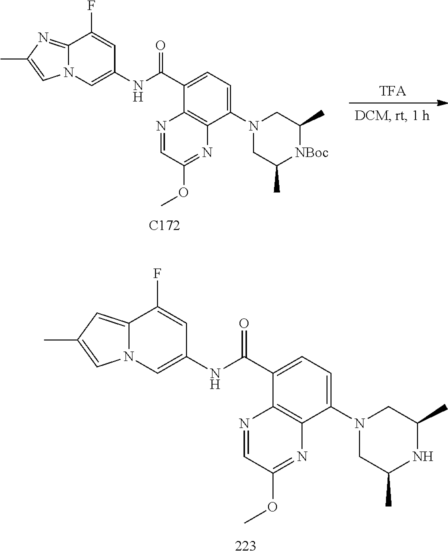

To a stirred solution of tert-butyl 4-[8-({8-fluoro-2-methylimidazo[1,2-a]pyridin-6-yl}carbamoyl)-3-methoxyquinoxalin-5-yl]-2,6-dimethylpiperazine-1-carboxylate (55 mg, 0.098 mmol, 1 equiv) in DCM (1 mL) was added TFA (1 mL) at room temperature. The resulting mixture was stirred for 1 h at room temperature. The resulting mixture was concentrated under vacuum. The residue was purified by Prep-HPLC (Condition 12, Gradient 1) to afford 8-(3,5-dimethylpiperazin-1-yl)-N-(8-fluoro-2-methylindolizin-6-yl)-2-methoxyquinoxaline-5-carboxamide; bis(trifluoroacetic acid) (23.9 mg, 35%) as a solid. LCMS (ES, m/z): 464[M+H]^{+ 1}H NMR (400 MHz, DMSO-d₆) δ 12.29 (d, J=2.8 Hz, 1H), 9.52-9.38 (m, 2H), 8.83 (d, J=1.3 Hz, 2H), 8.32-8.25 (m, 1H), 8.20-8.15 (m, 1H), 7.86 (d, J=12.0 Hz, 1H), 7.42 (d, J=8.4 Hz, 1H), 4.29-4.21 (m, 2H), 4.10 (s, 3H), 3.61 (d, J=10.4 Hz, 2H), 2.97 (dd, J=13.2, 11.0 Hz, 2H), 2.45 (s, 3H), 1.34 (d, J=6.5 Hz, 6H).

### Example 129: Synthesis of Compound 224

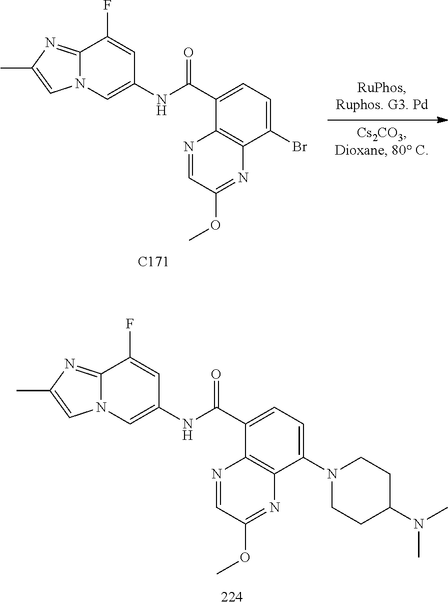

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv) and N,N-dimethylpiperidin-4-amine (35.76 mg, 0.278 mmol, 1.2 equiv), Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred for 2 days at 80° C. under nitrogen atmosphere. The resulting mixture was concentrated under vacuum. The residue was purified by silica gel column chromatography, eluted with CH₂Cl₂/MeOH (10:1) to afford 8-[4-(dimethylamino)piperidin-1-yl]-N-{8-fluoro-2-methylimidazo[1,2-a]pyridin-6-yl}-2-methoxyquinoxaline-5-carboxamide (50 mg, crude). The crude product was purified by Prep-HPLC (Condition 12, Gradient 1) to afford 8-[4-(dimethylamino)piperidin-1-yl]-N-{8-fluoro-2-methylimidazo[1,2-a]pyridin-6-yl}-2-methoxyquinoxaline-5-carboxamide; trifluoroacetic acid (22 mg, 16%) as a solid. LCMS (ES, m/z): 478[M+H]^{+ 1}H NMR (400 MHz, DMSO-d₆) δ 12.23 (s, 1H), 9.59 (s, 1H), 9.41 (s, 1H), 8.83 (d, J=1.0 Hz, 1H), 8.30 (d, J=8.3 Hz, 1H), 8.07 (s, 1H), 7.68 (s, 1H), 7.35 (d, J=8.4 Hz, 1H), 4.23 (d, J=12.0 Hz, 2H), 4.12 (s, 3H), 3.44 (s, 1H), 2.93 (t, J=12.0 Hz, 2H), 2.85 (d, J=4.9 Hz, 6H), 2.41 (s, 3H), 2.19 (d, J=11.7 Hz, 2H), 1.99-1.88 (m, 2H).

### Example 130: Synthesis of Compound 225

### Synthesis of Intermediate

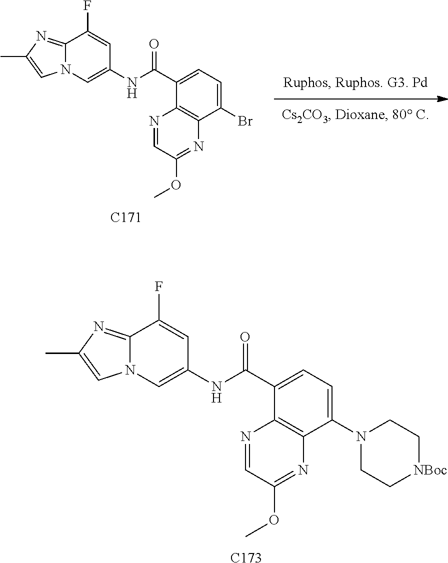 [SMILES: COc1cnc2c(C(=O)Nc3cc(F)c4nc(C)cn4c3)ccc(Br)c2n1 ; "C171" ; COc1cnc2c(C(=O)Nc3cc(F)c4nc(C)cn4c3)ccc(N3CCN(C(=O)OC(C)(C)C)CC3)c2n1 ; "C173"]

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv) and tert-butyl piperazine-1-carboxylate (51.95 mg, 0.278 mmol, 1.2 equiv), Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (50:1) to afford tert-butyl 4-[8-({8-fluoro-2-methylimidazo[1,2-a]pyridin-6-yl}carbamoyl)-3-methoxyquinoxalin-5-yl]piperazine-1-carboxylate (70 mg, 56%) as a solid. LCMS (ES, m/z): 534[M−H]⁻

### Synthesis of Compound 225

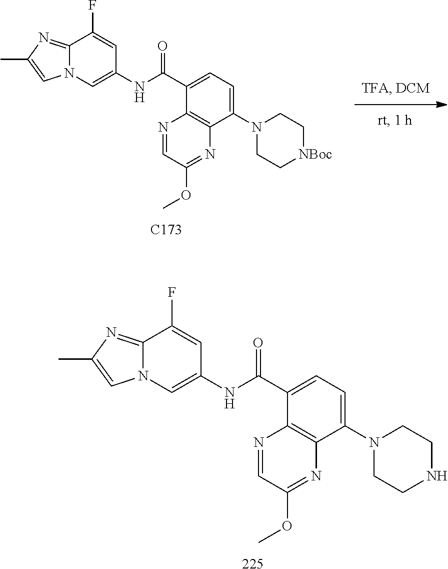

To a stirred solution of tert-butyl 4-[8-({8-fluoro-2-methylimidazo[1,2-a]pyridin-6-yl}carbamoyl)-3-methoxyquinoxalin-5-yl]piperazine-1-carboxylate (60 mg, 0.112 mmol, 1 equiv) in DCM (1 mL) was added TFA (1 mL) at room temperature. The resulting mixture was stirred for 1 h at room temperature. The resulting mixture was concentrated under vacuum. The residue was purified by Prep-HPLC (Condition 12, Gradient 1) to afford N-{8-fluoro-2-methylimidazo[1,2-a]pyridin-6-yl}-2-methoxy-8-(piperazin-1-yl)quinoxaline-5-carboxamide; trifluoroacetic acid (28.3 mg, 46%) as a solid. LCMS (ES, m/z): 436[M+H]^{+ 1}H NMR (400 MHz, DMSO-d₆) δ 12.20 (d, J=12.6 Hz, 1H), 9.47 (s, 1H), 9.03-8.81 (m, 3H), 8.29 (dd, J=8.3, 1.2 Hz, 1H), 8.14 (s, 1H), 7.77 (s, 1H), 7.41 (d, J=8.3 Hz, 1H), 4.11 (s, 3H), 3.73-3.66 (m, 4H), 3.43-3.38 (m, 4H), 2.43 (d, J=4.9 Hz, 3H).

### Example 131: Synthesis of Compound 226

### Synthesis of Intermediate C174

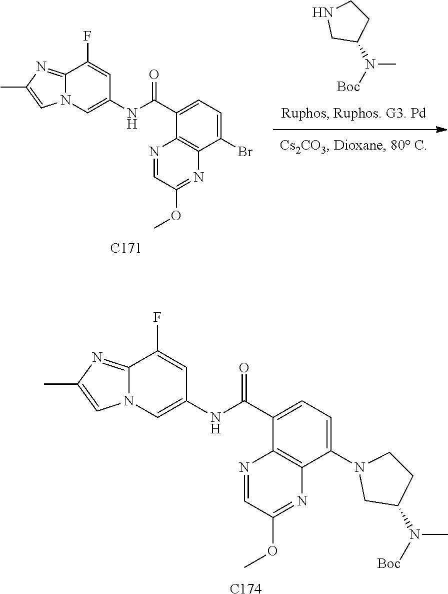

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv) and tert-butyl (S)-methyl(pyrrolidin-3-yl)carbamate (55.86 mg, 0.278 mmol, 1.2 equiv), Cs₂CO₃ (227.19 mg, 0.696 mmol, 3 equiv) in 1,4-dioxane (3 mL) was added Ruphos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (50:1) to afford tert-butyl (S)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (55 mg, 43%) as a solid. LCMS (ES, m/z): 550[M−H]⁻

### Synthesis of Compound 226

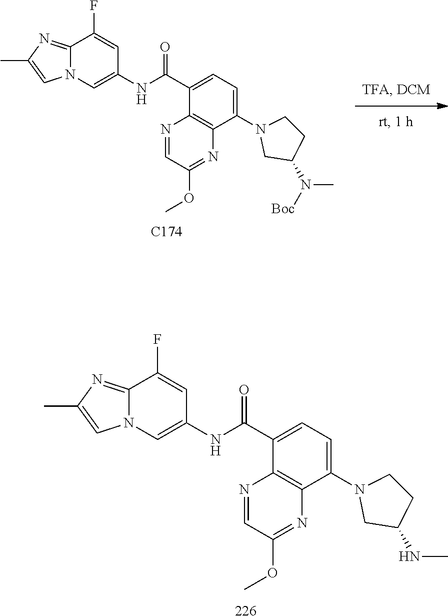

To a stirred solution of tert-butyl (S)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (50 mg, 0.091 mmol, 1 equiv) in DCM (1 mL) was added TFA (1 mL) at room temperature. The resulting mixture was stirred for 1 h at room temperature. The resulting mixture was concentrated under vacuum. The residue was purified by Prep-HPLC (Condition 12, Gradient 1) to afford (S)—N-{8-fluoro-2-methylimidazo[1,2-a]pyridin-6-yl}-2-methoxy-8-[3-(methylamino)pyrrolidin-1-yl]quinoxaline-5-carboxamide; trifluoroacetic acid (10.4 mg, 20%) as a solid. LCMS (ES, m/z): 450[M+H]⁺ 1H NMR (400 MHz, DMSO-d₆) δ 12.50 (s, 1H), 9.43 (s, 1H), 8.81 (s, 3H), 8.34 (d, J=8.7 Hz, 1H), 8.08 (d, J=2.6 Hz, 1H), 7.75 (d, J=12.2 Hz, 1H), 6.96 (d, J=8.9 Hz, 1H), 4.26 (qd, J=12.4, 5.5 Hz, 2H), 4.09 (s, 3H), 3.96 (tt, J=12.7, 6.5 Hz, 2H), 3.82 (dt, J=11.0, 7.4 Hz, 1H), 2.72-2.65 (m, 3H), 2.44-2.33 (m, 4H), 2.21 (dq, J=13.1, 6.7 Hz, 1H).

### Example 132: Synthesis of Compound 227

### Synthesis of Intermediate C175

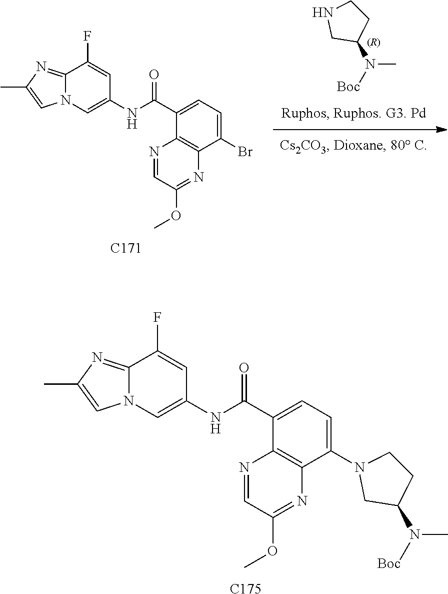

To a stirred mixture of 8-bromo-N-{8-fluoro-2-methylimidazo[1,2-a]pyridin-6-yl}-2-methoxyquinoxaline-5-carboxamide (100 mg, 0.232 mmol, 1 equiv) and tert-butyl-(R)-methyl(pyrrolidin-3-yl)carbamate (55.86 mg, 0.278 mmol, 1.2 equiv),Cs₂CO₃ (227.19 mg, 0.696 mmol, 3 equiv) in 1,4-dioxane (3 mL) was added RuPhos (10.85 mg, 0.023 mmol, 0.1 equiv) and RuPhos Palladacycle Gen.3 (19.44 mg, 0.023 mmol, 0.1 equiv) at room temperature under nitrogen atmosphere. The resulting mixture was stirred overnight at 80° C. under nitrogen atmosphere. The resulting mixture was concentrated under vacuum. The residue was purified by silica gel column chromatography, eluted with CH₂Cl₂/MeOH (50:1) to afford tert-butyl (R)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (60 mg, 43%) as a solid. LCMS (ES, m/z): 550[M−H]⁺

### Synthesis of Compound 227

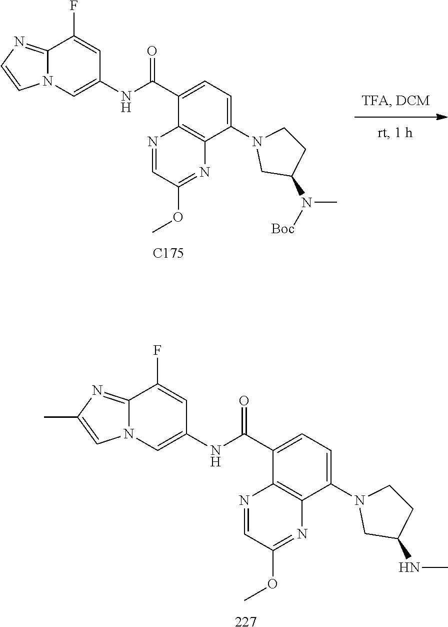

To a stirred solution of tert-butyl (R)-(1-(8-((8-fluoro-2-methylimidazo[1,2-a]pyridin-6-yl)carbamoyl)-3-methoxyquinoxalin-5-yl)pyrrolidin-3-yl)(methyl)carbamate (50 mg, 0.091 mmol, 1 equiv) in DCM (1 mL) was added TFA (1 mL) at room temperature. The resulting mixture was stirred for 1 h at room temperature. The resulting mixture was concentrated under vacuum. The residue was purified by Prep-HPLC (Condition 12, Gradient 1) to afford (R)—N-(8-fluoro-2-methylimidazo[1,2-a]pyridin-6-yl)-2-methoxy-8-(3-(methylamino)pyrrolidin-1-yl)quinoxaline-5-carboxamide 2,2,2-trifluoroacetate (12.3 mg, 24%) as a solid. LCMS (ES, m/z): 450[M+H]⁺ 1H NMR (400 MHz, DMSO-d₆) δ 12.50 (s, 1H), 9.43 (s, 1H), 8.81 (s, 3H), 8.33 (d, J=8.7 Hz, 1H), 8.09 (d, J=2.5 Hz, 1H), 7.77 (d, J=12.2 Hz, 1H), 6.96 (d, J=8.8 Hz, 1H), 4.26 (qd, J=12.4, 5.6 Hz, 2H), 4.09 (s, 3H), 4.03-3.91 (m, 2H), 3.87-3.76 (m, 1H), 2.69 (d, J=10.3 Hz, 3H), 2.44-2.33 (m, 4H), 2.21 (dq, J=13.4, 6.9 Hz, 1H).

### Example 133: Synthesis of Compound 238

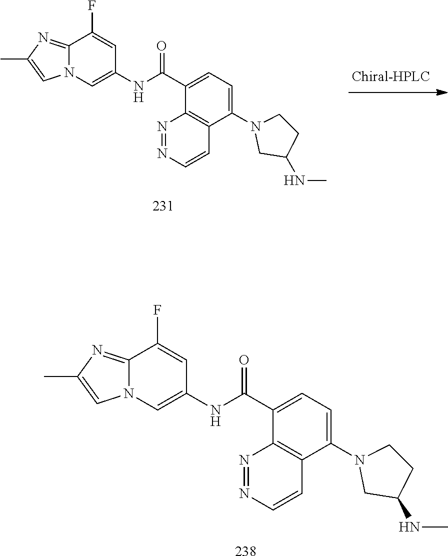

35 mg of N-(8-fluoro-2-methylimidazo[1,2-a]pyridin-6-yl)-5-(3-(methylamino)pyrrolidin-1-yl) cinnoline-8-carboxamide was separated by the following conditions (Column: CHIRALPAK IG, 3*25 cm, 5 m; Mobile Phase A: Hex: DCM=1: 1--HPLC, Mobile Phase B: EtOH (0.1% IPAmine)-HPLC; Flow rate: 35 mL/min; Gradient: 50% B to 50% B in 50 min; Wave Length: 220/254 nm; RT1(min): 33; RT2(min): 41; Sample Solvent: EtOH--HPLC; Injection Volume: 1.5 mL; Number Of Runs: 7) to yield (R)—N-(8-fluoro-2-methylimidazo[1,2-a]pyridin-6-yl)-5-(3-(methylamino)pyrrolidin-1-yl)cinnoline-8-carboxamide (10 mg, 28.57%) as a brown solid. LCMS (ES, m/z): 420 [M+H]⁺ retention time: 4.371 ¹H NMR (400 MHz, DMSO-d₆) δ 12.72 (s, 1H), 9.35 (d, J=6.1 Hz, 1H), 9.20 (d, J=1.6 Hz, 1H), 8.64 (d, J=6.1 Hz, 1H), 8.56 (d, J=8.6 Hz, 1H), 7.91 (d, J=3.0 Hz, 1H), 7.34 (dd, J=12.5, 1.7 Hz, 1H), 6.94 (d, J=8.8 Hz, 1H), 3.96-3.80 (m, 2H), 3.69 (q, J=8.4, 7.5 Hz, 1H), 3.51 (dd, J=10.3, 4.0 Hz, 1H), 2.50-2.49 (m, 1H), 2.36 (s, 3H), 2.32 (s, 3H), 2.12 (dt, J=12.6, 6.3 Hz, 1H), 1.91 (dt, J=11.8, 6.0 Hz, 1H).

### Example 134: Synthesis of Compound 239

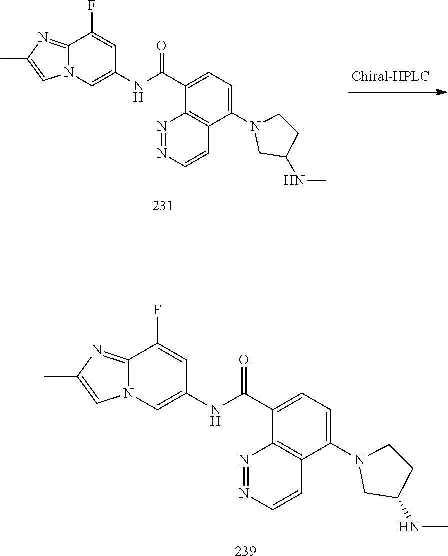

35 mg of N-(8-fluoro-2-methylimidazo[1,2-a]pyridin-6-yl)-5-(3-(methylamino)pyrrolidin-1-yl) cinnoline-8-carboxamide was separated by the following conditions (Column: CHIRALPAK IG, 3*25 cm, 5 m; Mobile Phase A: Hex: DCM=1: 1--HPLC, Mobile Phase B: EtOH(0.1% IPAmine)-HPLC; Flow rate: 35 mL/min; Gradient: 50% B to 50% B in 50 min; Wave Length: 220/254 nm; RT1(min): 33; RT2(min): 41; Sample Solvent: EtOH--HPLC; Injection Volume: 1.5 mL; Number Of Runs: 7) to yield (S)—N-(8-fluoro-2-methylimidazo[1,2-a]pyridin-6-yl)-5-(3-(methylamino)pyrrolidin-1-yl)cinnoline-8-carboxamide (10 mg, 28.57) as a solid. LCMS (ES, m/z): 420 [M+H]⁺ retention time: 4.998′1H NMR (400 MHz, DMSO-d₆) δ 12.72 (s, 1H), 9.35 (d, J=6.1 Hz, 1H), 9.20 (d, J=1.6 Hz, 1H), 8.64 (d, J=6.1 Hz, 1H), 8.56 (d, J=8.6 Hz, 1H), 7.91 (d, J=3.0 Hz, 1H), 7.34 (dd, J=12.5, 1.7 Hz, 1H), 6.94 (d, J=8.8 Hz, 1H), 3.96-3.80 (m, 2H), 3.69 (q, J=8.4, 7.5 Hz, 1H), 3.51 (dd, J=10.3, 4.0 Hz, 1H), 2.50-2.49 (m, 1H), 2.36 (s, 3H), 2.32 (s, 3H), 2.12 (dt, J=12.6, 6.3 Hz, 1H), 1.91 (dt, J=11.8, 6.0 Hz, 1H).

### Example 135: Synthesis of Compound 244

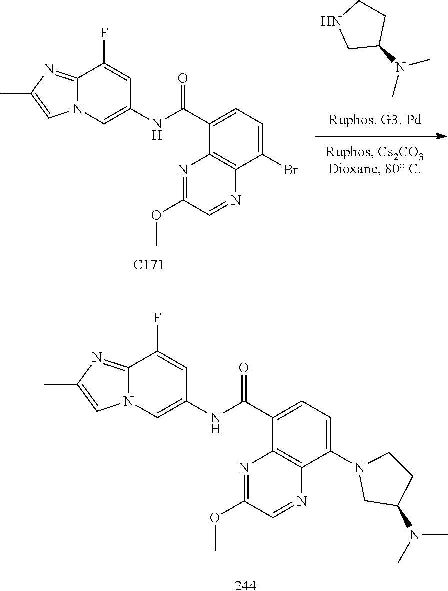

To a stirred mixture of 8-bromo-N-{8-fluoro-2-methylimidazo[1,2-a]pyridin-6-yl}-3-methoxyquinoxaline-5-carboxamide (90 mg, 0.209 mmol, 1 equiv) and (3R)—N,N-dimethylpyrrolidin-3-amine (31.05 mg, 0.272 mmol, 1.3 equiv) in dioxane (2 mL) were added Cs₂CO₃ (204.47 mg, 0.627 mmol, 3 equiv), Ruphos (19.52 mg, 0.042 mmol, 0.2 equiv) and RuPhos Palladacycle Gen.3 (17.50 mg, 0.021 mmol, 0.1 equiv) at room temperature under nitrogen atmosphere. The resulting mixture was stirred overnight at 80° C. under nitrogen atmosphere. The resulting mixture was concentrated under reduced pressure. The residue was purified by silica gel column chromatography eluted with DCM/MeOH (20:1) to afford crude product. The crude product was purified by Prep-HPLC (Condition 1, Gradient 14) to afford 8-[(3R)-3-(dimethylamino)pyrrolidin-1-yl]-N-{8-fluoro-2-methylimidazo[1,2-a]pyridin-6-yl}-3-methoxyquinoxaline-5-carboxamide (9.5 mg, 10%) as a solid. LCMS (ES, m/z): 464[M+H]⁺0 ¹H NMR (400 MHz, Methanol-d₄) δ 8.64 (d, J=1.7 Hz, 1H), 8.18 (d, J=8.9 Hz, 1H), 8.05 (s, 1H), 7.45-7.39 (m, 1H), 6.52-6.43 (m, 2H), 4.04 (s, 3H), 3.89-3.75 (m, 2H), 3.64-3.52 (m, 1H), 2.82 (q, J=8.1 Hz, 1H), 2.37 (d, J=7.9 Hz, 9H), 2.24 (dt, J=12.4, 6.5 Hz, 1H), 1.84 (p, J=10.6 Hz, 1H).

### Example 136: Synthesis of Compound 246

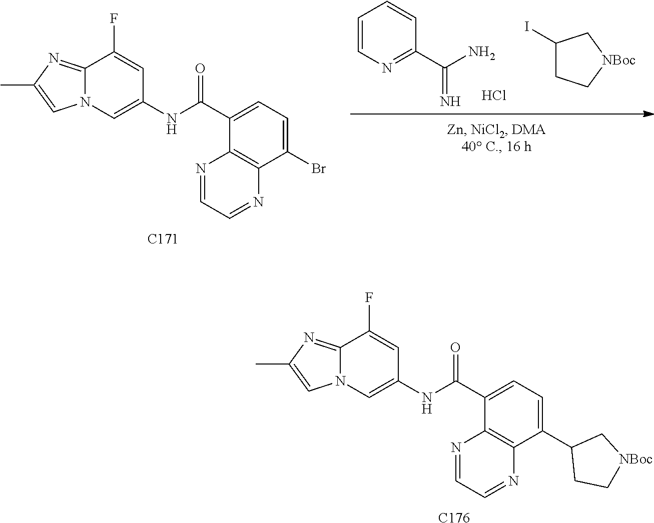

To a stirred mixture of tert-butyl 3-iodopyrrolidine-1-carboxylate (1299.2 mg, 4.372 mmol, 3.5 equiv) in DMA (5 mL) were added NiCl₂ (161.9 mg, 1.249 mmol, 1.0 equiv) and pyridine-2-carboximidamide (30.3 mg, 0.250 mmol, 0.2 equiv) in portions at room temperature under nitrogen atmosphere. The resulting mixture was stirred for 2 h at 40° C. under nitrogen atmosphere. To the above mixture were added Zn (245.1 mg, 3.747 mmol, 3.0 equiv) and 8-bromo-N-{8-fluoro-2-methylimidazo[1,2-a]pyridin-6-yl}quinoxaline-5-carboxamide (500 mg, 1.249 mmol, 1.0 equiv) at room temperature under nitrogen atmosphere. The resulting mixture was stirred for additional 16 h at 40° C. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 3-[8-({8-fluoro-2-methylimidazo[1,2-a]pyridin-6-yl}carbamoyl)quinoxalin-5-yl]pyrrolidine-1-carboxylate (120 mg, 4.50%) as a solid.

LCMS (ES, m/z):491 [M+H]⁺

### Example 137: Synthesis of Compound 248

### Synthesis of Intermediate C177

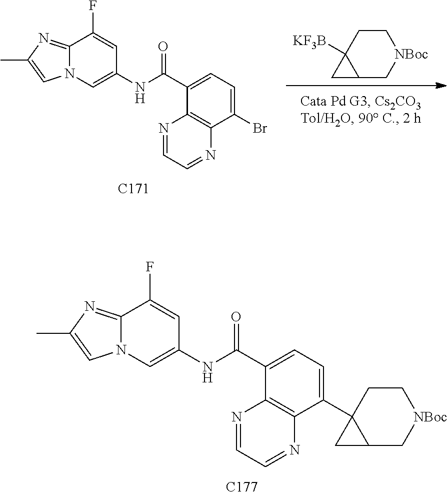

To a stirred mixture of 8-bromo-N-{8-fluoro-2-methylimidazo[1,2-a]pyridin-6-yl}quinoxaline-5-carboxamide (80 mg, 0.200 mmol, 1 equiv) and tert-butyl 6-(trifluoro-lambda4-boranyl)-3-azabicyclo[4.1.0]heptane-3-carboxylate potassium (66.7 mg, 0.220 mmol, 1.1 equiv) in Toluene (3 mL) and H₂O (0.3 mL) were added Cs₂CO₃ (97.7 mg, 0.300 mmol, 1.5 equiv) and cata Pd G3 (14.6 mg, 0.020 mmol, 0.1 equiv) at room temperature under nitrogen atmosphere. The resulting mixture was stirred for 2 h at 90° C. under nitrogen atmosphere. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with EA to afford tert-butyl 6-[8-({8-fluoro-2-methylimidazo[1,2-a]pyridin-6-yl}carbamoyl)quinoxalin-5-yl]-3-azabicyclo[4.1.0]heptane-3-carboxylate (90 mg, 87%) as a solid. LCMS (ES, m/z):516 [M+H]⁺

### Synthesis of Compound 248

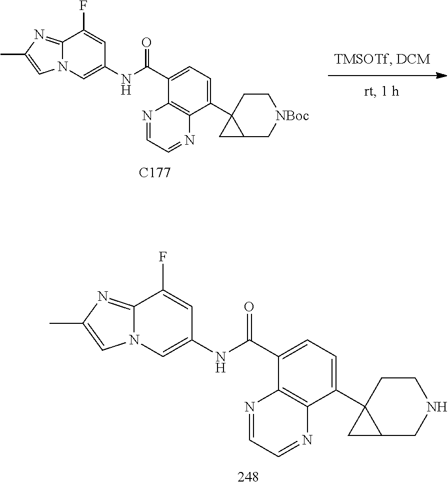

To a stirred mixture of tert-butyl 6-[8-({8-fluoro-2-methylimidazo[1,2-a]pyridin-6-yl}carbamoyl)quinoxalin-5-yl]-3-azabicyclo[4.1.0]heptane-3-carboxylate (70 mg, 0.136 mmol, 1 equiv) in DCM (1 mL) was added TMSOTf (120.5 mg, 0.544 mmol, 4 equiv) dropwise at room temperature. The resulting mixture was stirred for 1 h at room temperature. The resulting mixture was concentrated under reduced pressure. The crude product was purified by Prep-HPLC (Condition 13, Gradient 1) to afford 8-{3-azabicyclo[4.1.0]heptan-6-yl}-N-{8-fluoro-2-methylimidazo[1,2-a]pyridin-6-yl}quinoxaline-5-carboxamide 2,2,2-trifluoroacetate (35.7 mg, 63.26%) as a solid. LCMS (ES, m/z):416 [M+H]^{+ 1}H NMR (400 MHz, DMSO-d₆) δ 11.98 (s, 1H), 9.42 (d, J=1.6 Hz, 1H), 9.19 (dd, J=15.4, 1.8 Hz, 2H), 8.69-8.59 (m, 1H), 8.54-8.53 (m, 1H), 8.41 (d, J=7.6 Hz, 1H), 8.10 (d, J=2.8 Hz, 1H), 7.97 (d, J=7.6 Hz, 1H), 7.61 (d, J=12.1 Hz, 1H), 3.77-3.66 (m, 1H), 3.36 (d, J=13.3 Hz, 1H), 3.25-3.17 (m, 1H), 3.08-3.00 (m, 1H), 2.47-2.42 (m, 4H), 2.14 (dt, J=14.0, 6.0 Hz, 1H), 1.58-1.48 (m, 1H), 1.32 (t, J=5.8 Hz, 1H), 1.26 (dd, J=9.3, 5.5 Hz, 1H).

### Example 138: Synthesis of Compound 250

### Synthesis of Intermediate C178

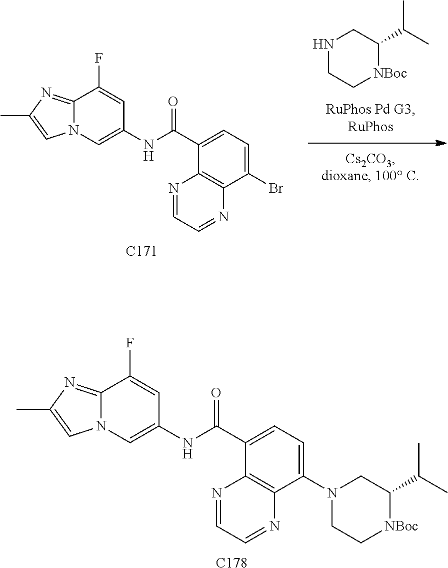 [SMILES: Cc1cn2cc(NC(=O)c3ccc(Br)c4nccnc34)cc(F)c2n1 ; CC(C)[C@H]1CNCCN1C(=O)OC(C)(C)C ; "C171" ; Cc1cn2cc(NC(=O)c3ccc(N4CCN(C(=O)OC(C)(C)C)[C@@H](C(C)C)C4)c4nccnc34)cc(F)c2n1 ; "C178"]

To a stirred solution of 8-bromo-N-{8-fluoro-2-methylimidazo[1,2-a]pyridin-6-yl}quinoxaline-5-carboxamide (150 mg, 0.375 mmol, 1 equiv) and tert-butyl (2S)-2-isopropylpiperazine-1-carboxylate (129 mg, 0.563 mmol, 1.5 equiv) in dioxane (5 mL) were added Cs₂CO₃ (244.24 mg, 0.750 mmol, 2 equiv), RuPhos (35 mg, 0.075 mmol, 0.2 equiv) and RuPhos Palladacycle Gen.3 (4 mg, 0.004 mmol, 0.1 equiv) at room temperature under nitrogen atmosphere. The resulting mixture was stirred for 3 h at 100° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl (2S)-4-[8-({8-fluoro-2-methylimidazo[1,2-a]pyridin-6-yl}carbamoyl)quinoxalin-5-yl]-2-isopropylpiperazine-1-carboxylate (100 mg, 49%) as a solid. LCMS (ES, m/z): 548 [M+H]

### Synthesis of Compound 250

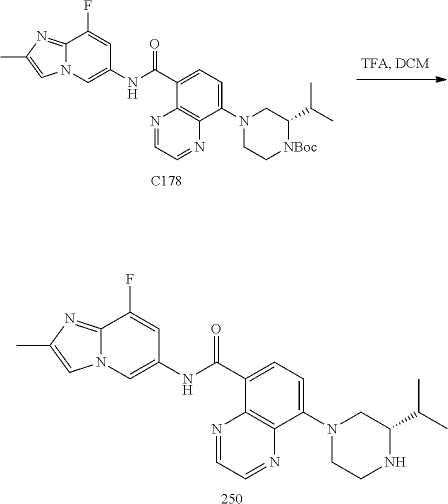

A solution of tert-butyl (2S)-4-[8-({8-fluoro-2-methylimidazo[1,2-a]pyridin-6-yl}carbamoyl)quinoxalin-5-yl]-2-isopropylpiperazine-1-carboxylate (100 mg, 0.183 mmol, 1 equiv) in DCM (3 mL) and TFA (1 mL) was stirred for 3 h at room temperature. The resulting mixture was concentrated under reduced pressure. The residue was basified to pH 8 with 7 M NH₃(g) in MeOH. The resulting mixture was concentrated under reduced pressure. The residue was purified by reverse flash chromatography (Condition 4, Gradient 1) to afford N-{8-fluoro-2-methylimidazo[1,2-a]pyridin-6-yl}-8-[(3S)-3-isopropylpiperazin-1-yl]quinoxaline-5-carboxamide (30 mg, 37%) as a solid. LCMS (ES, m/z): 448 [M+H]^{+ 1}H NMR (300 MHz, DMSO-d₆) δ 12.41 (s, 1H), 9.27 (s, 1H), 9.12 (s, 1H), 9.02 (s, 1H), 8.52 (d, J=8.5 Hz, 1H), 7.92 (s, 1H), 7.45 (d, J=12.6 Hz, 1H), 7.34 (d, J=8.6 Hz, 1H), 4.10 (s, 1H), 4.04 (s, 1H), 3.08 (s, 3H), 2.98 (s, 2H), 2.72 (s, 1H), 2.35 (s, 3H), 1.66 (s, 1H), 0.98 (t, J=6.8 Hz, 6H).

### Example 139: Synthesis of Compound 252

### Synthesis of Intermediate C179

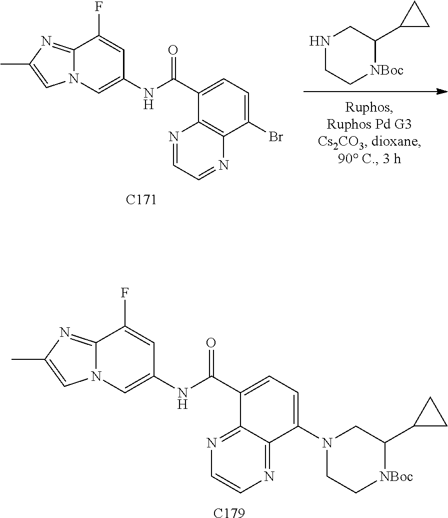 [SMILES: Cc1cn2cc(NC(=O)c3ccc(Br)c4nccnc34)cc(F)c2n1 ; CC(C)(C)OC(=O)N1CCNCC1C1CC1 ; "C171" ; Cc1cn2cc(NC(=O)c3ccc(N4CCN(C(=O)OC(C)(C)C)C(C5CC5)C4)c4nccnc34)cc(F)c2n1 ; "C179"]

To a stirred mixture of 8-bromo-N-{8-fluoro-2-methylimidazo[1,2-a]pyridin-6-yl}quinoxaline-5-carboxamide (130 mg, 0.325 mmol, 1 equiv) and tert-butyl 2-cyclopropylpiperazine-1-carboxylate (110.2 mg, 0.488 mmol, 1.5 equiv) in dioxane (1 mL) were added Cs₂CO₃ (317.5 mg, 0.975 mmol, 3.0 equiv), Ruphos (30.3 mg, 0.065 mmol, 0.2 equiv) and RuPhos Palladacycle Gen.3 (27.2 mg, 0.033 mmol, 0.1 equiv) at room temperature. The resulting mixture was stirred for 3 h at 90° C. under nitrogen atmosphere. The mixture was allowed to cool down to room temperature. The residue was purified by silica gel column chromatography, eluted with CH₂Cl₂/MeOH (10:1) to afford tert-butyl 2-cyclopropyl-4-[8-({8-fluoro-2-methylimidazo[1,2-a]pyridin-6-yl}carbamoyl)quinoxalin-5-yl]piperazine-1-carboxylate (108 mg, 61%) as a solid. LCMS (ES, m/z): 546 [M+H]⁺

### Synthesis of Compound 252

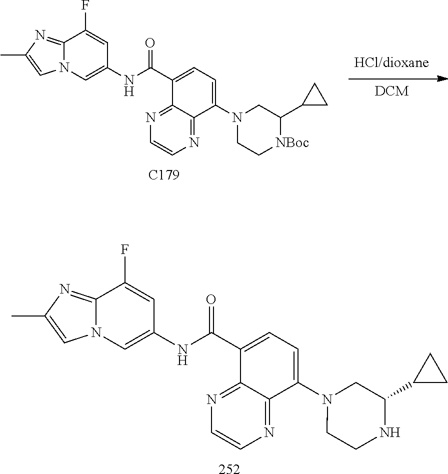

A solution of tert-butyl 2-cyclopropyl-4-[8-({8-fluoro-2-methylimidazo[1,2-a]pyridin-6-yl}carbamoyl)quinoxalin-5-yl]piperazine-1-carboxylate (103 mg, 0.189 mmol, 1 equiv) in DCM (0.8 mL) was treated with HCl(gas) in 1,4-dioxane (0.2 mL). The resulting mixture was stirred for 1 h at room temperature. The resulting mixture was concentrated under reduced pressure. The residue was neutralized to PH=7 with NaHCO₃ aqueous and extracted with DCM/MeOH (20/1). The organic layer was concentrated in vacuo. The residue (80 mg) was purified by prep-chiral-HPLC (Condition 3, Gradient 1) to afford 8-[(3S)-3-cyclopropylpiperazin-1-yl]-N-{8-fluoro-2-methylimidazo[1,2-a]pyridin-6-yl}quinoxaline-5-carboxamide (14 mg, 23%) as a solid. LCMS (ES, m/z): 446 [M+H]^{+ 1}H NMR (400 MHz, DMSO-d₆) δ 12.42 (s, 1H), 9.28 (d, J=1.7 Hz, 1H), 9.12 (d, J=1.8 Hz, 1H), 9.03 (d, J=1.8 Hz, 1H), 8.53 (d, J=8.5 Hz, 1H), 7.92 (d, J=3.1 Hz, 1H), 7.46 (dd, J=12.4, 1.7 Hz, 1H), 7.34 (d, J=8.6 Hz, 1H), 4.06 (d, J=9.9 Hz, 2H), 3.10-2.92 (m, 3H), 2.86 (t, J=10.8 Hz, 1H), 2.36 (s, 3H), 2.21 (t, J=8.4 Hz, 1H), 0.87-0.73 (m, 1H), 0.44 (d, J=8.4 Hz, 2H), 0.38-0.24 (m, 2H).

### Example 140: Synthesis of Compound 253

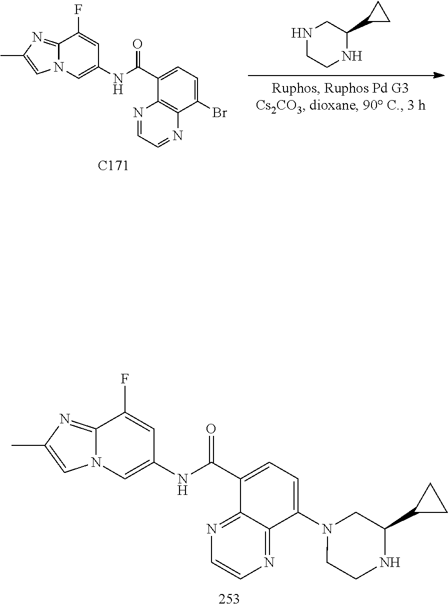

To a stirred mixture of 8-bromo-N-{8-fluoro-2-methylimidazo[1,2-a]pyridin-6-yl}quinoxaline-5-carboxamide (100 mg, 0.250 mmol, 1.0 equiv) and (2R)-2-cyclopropylpiperazine (47.3 mg, 0.375 mmol, 1.5 equiv) in dioxane (1 mL) were added Cs₂CO₃ (244.2 mg, 0.750 mmol, 3 equiv), Ruphos (23.3 mg, 0.050 mmol, 0.2 equiv) and RuPhos Palladacycle Gen.3 (20.9 mg, 0.025 mmol, 0.1 equiv) at room temperature. The resulting mixture was stirred for 3 h at 90° C. under nitrogen atmosphere. The mixture was allowed to cool down to room temperature. The reaction mixture was diluted with water (3 mL), and extracted with DCM/MeOH (20/1) (3 mL). The organic layer was dried by anhydrous Nₐ₂SO₄, filtered and the filtrate was concentrated off in vacuo. The crude product was purified by Prep-HPLC (Condition 1, Gradient 24) to afford 8-[(3R)-3-cyclopropylpiperazin-1-yl]-N-{8-fluoro-2-methylimidazo[1,2-a]pyridin-6-yl}quinoxaline-5-carboxamide (16.5 mg, 15.27%) as a a solid. LCMS (ES, m/z): 446 [M+H]^{+ 1}H NMR (400 MHz, DMSO-d₆) δ 12.43 (s, 1H), 9.28 (d, J=1.6 Hz, 1H), 9.12 (d, J=1.8 Hz, 1H), 9.03 (d, J=1.8 Hz, 1H), 8.52 (d, J=8.5 Hz, 1H), 7.92 (d, J=3.0 Hz, 1H), 7.46 (dd, J=12.6, 1.6 Hz, 1H), 7.33 (d, J=8.6 Hz, 1H), 4.06 (d, J=9.7 Hz, 2H), 2.97 (td, J=20.6, 18.8, 9.5 Hz, 3H), 2.84 (t, J=10.8 Hz, 1H), 2.36 (s, 3H), 2.18 (t, J=9.2 Hz, 1H), 0.89-0.73 (m, 1H), 0.43 (d, J=8.1 Hz, 2H), 0.37-0.25 (m, 2H).

### Example 141: Synthesis of Compound 255

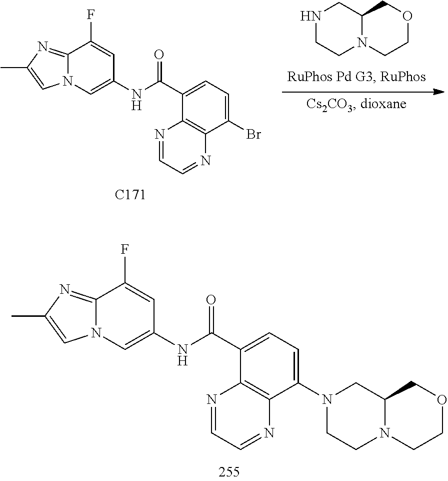

To a stirred solution of 8-bromo-N-(8-fluoro-2-methylimidazo[1,2-a]pyridin-6-yl)quinoxaline-5-carboxamide (140 mg, 0.351 mmol, 1 equiv) and (S)-octahydropyrazino[2,1-c][1,4]oxazine (100 mg, 0.702 mmol, 2 equiv) in dioxane (10 mL) were added Cs₂CO₃ (229 mg, 0.702 mmol, 2 equiv), RuPhos (33 mg, 0.07 mmol, 0.2 equiv) and RuPhos Palladacycle Gen.3 (29 mg, 0.035 mmol, 0.1 equiv) at room temperature under nitrogen atmosphere. The resulting mixture was stirred for 3 h at 100° C. under nitrogen atmosphere. The mixture was allowed to cool down to room temperature. The resulting mixture was concentrated under reduced pressure. The residue was purified by reverse flash chromatography (Condition 5, Gradient 3) afford (S)—N-(8-fluoro-2-methylimidazo[1,2-a]pyridin-6-yl)-8-(hexahydropyrazino[2,1-c][1,4]oxazin-8(1H)-yl)quinoxaline-5-carboxamide (11 mg, 6.8%) as a solid. LCMS (ES, m/z): 462 [M+H]^{+ 1}H NMR (300 MHz, DMSO-d₆) δ 12.37 (s, 1H), 9.27 (d, J=1.6 Hz, 1H), 9.13 (d, J=1.8 Hz, 1H), 9.04 (d, J=1.9 Hz, 1H), 8.51 (d, J=8.4 Hz, 1H), 7.92 (d, J=3.1 Hz, 1H), 7.50-7.39 (m, 1H), 7.34 (d, J=8.5 Hz, 1H), 4.21 (d, J=11.8 Hz, 1H), 3.91 (d, J=11.1 Hz, 1H), 3.77 (dd, J=17.0, 10.6 Hz, 2H), 3.58 (t, J=11.2 Hz, 1H), 3.16 (dt, J=23.5, 10.9 Hz, 2H), 2.86 (d, J=11.1 Hz, 1H), 2.70 (t, J=11.1 Hz, 2H), 2.45 (s, 1H), 2.35 (s, 5H).

### Example 142: Synthesis of Compound 256

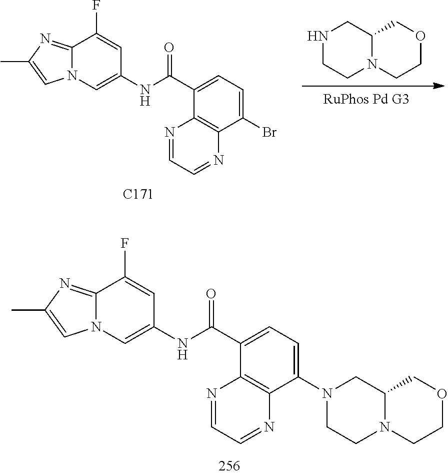

To a stirred solution of 8-bromo-N-(8-fluoro-2-methylimidazo[1,2-a]pyridin-6-yl)quinoxaline-5-carboxamide (150 mg, 0.375 mmol, 1 equiv), Cs₂CO₃ (366 mg, 1.125 mmol, 3 equiv) and (R)-octahydropyrazino[2,1-c][1,4]oxazine (79 mg, 0.563 mmol, 1.5 equiv) in 1,4-dioxane (5 mL) were added RuPhos (34 mg, 0.075 mmol, 0.2 equiv) and RuPhos Palladacycle Gen.3 (30 mg, 0.036 mmol, 0.1 equiv) at room temperature under nitrogen atmosphere. The resulting mixture was stirred for 5 h at 100° C. under nitrogen atmosphere. The resulting mixture was concentrated under reduced pressure. The residue was purified by reverse flash chromatography (Condition 5, Gradient 1) to afford (R)—N-(8-fluoro-2-methylimidazo[1,2-a]pyridin-6-yl)-8-(hexahydropyrazino[2,1-c][1,4]oxazin-8(1H)-yl)quinoxaline-5-carboxamide (60 mg, 35%) as a solid. LCMS (ES, m/z): 462 [M+H]^{+ 1}H NMR (400 MHz, DMSO-d₆) δ 12.37 (s, 1H), 9.27 (s, 1H), 9.13 (s, 1H), 9.05 (s, 1H), 8.51 (d, J=8.4 Hz, 1H), 7.93 (s, 1H), 7.45 (d, J=12.5 Hz, 1H), 7.34 (d, J=8.5 Hz, 1H), 4.22 (d, J=11.8 Hz, 1H), 3.92 (d, J=11.7 Hz, 1H), 3.81 (d, J=11.2 Hz, 2H), 3.75 (d, J=10.7 Hz, 1H), 3.58 (t, J=11.1 Hz, 1H), 3.17 (dt, J=32.3, 11.0 Hz, 1H), 2.91 (t, J=11.1 Hz, 1H), 2.87 (d, J=11.2 Hz, 2H), 2.70 (t, J=11.4 Hz, 1H), 2.36 (s, 3H), 2.30 (d, J=11.1 Hz, 1H).

### Example 143: Synthesis of Compound 318

### Synthesis of Intermediate C180

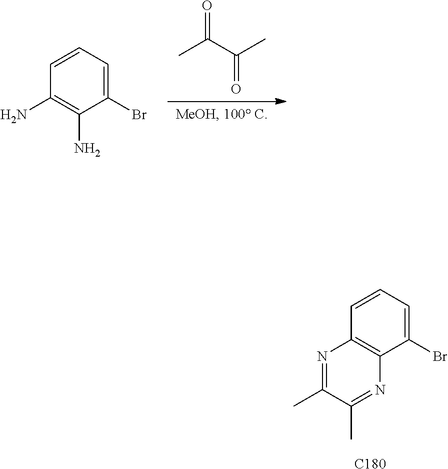

A solution of 3-bromobenzene-1,2-diamine (20 g, 106.929 mmol, 1 equiv) and diacetyl (11.05 g, 128.315 mmol, 1.2 equiv) in acetic acid (100 mL) was stirred for 2 h at 100° C. The reaction was quenched with water (500 mL) at room temperature. The precipitated solids were collected by filtration and washed with water (2×50 mL), dried to afford 5-bromo-2,3-dimethylquinoxaline (15 g, 59%) as a solid. LCMS (ES, m/z): 237 [M+H]⁺

### Synthesis of Intermediate C181

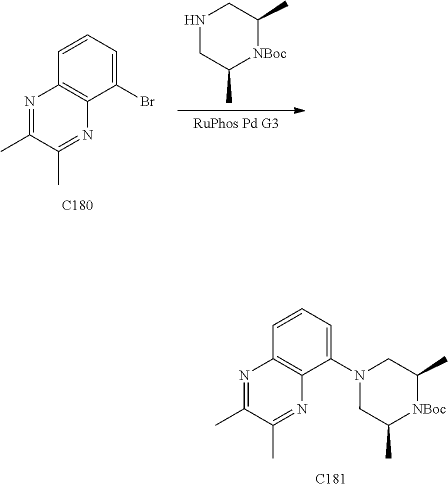

To a solution of 5-bromo-2,3-dimethylquinoxaline (2.0 g, 8.435 mmol, 1 equiv) and cis-tert-butyl (2R,6S)-2,6-dimethylpiperazine-1-carboxylate (2.71 g, 12.652 mmol, 1.5 equiv) in 1,4-dioxane (20 mL) were added cesium carbonate (5.50 g, 16.870 mmol, 2.0 equiv), RuPhos (0.39 g, 0.844 mmol, 0.1 equiv) and RuPhos Palladacycle Gen.3 (0.71 g, 0.844 mmol, 0.1 equiv). After stirring for 2 h at 90° C. under a nitrogen atmosphere, the resulting mixture was concentrated under reduced pressure. The residue was purified by silica gel column chromatography, eluted with PE:EA (1:1) to afford cistert-butyl (2R,6S)-4-(2,3-dimethylquinoxalin-5-yl)-2,6-dimethylpiperazine-1-carboxylate (1.8 g, 58%) as a solid. LCMS (ES, m/z): 371 [M+H]⁺

### Synthesis of Intermediate C182

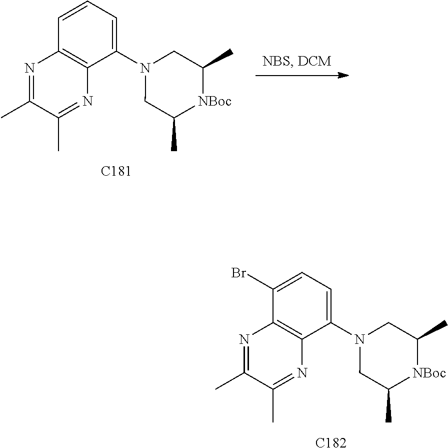

To a stirred solution of cis-tert-butyl (2R,6S)-4-(2,3-dimethylquinoxalin-5-yl)-2,6-dimethylpiperazine-1-carboxylate (1.2 g, 3.239 mmol, 1 equiv) in DCM (10 mL) was added NBS (0.58 g, 3.271 mmol, 1.01 equiv) in portions at 0° C. The resulting mixture was stirred for 2 h at room temperature. The resulting mixture was diluted with water (50 mL). The resulting mixture was extracted with EA (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:1) to afford cis-tert-butyl (2R,6S)-4-(8-bromo-2,3-dimethylquinoxalin-5-yl)-2,6-dimethylpiperazine-1-carboxylate (0.9 g, 62%) as a solid. LCMS (ES, m/z): 451 [M+H]⁺

### Synthesis of Intermediate C183

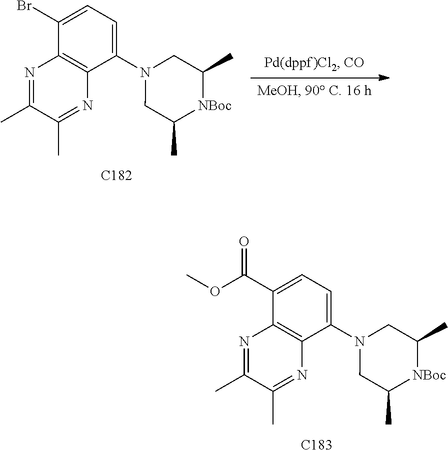

To a solution of cis-tert-butyl (2R,6S)-4-(8-bromo-2,3-dimethylquinoxalin-5-yl)-2,6-dimethylpiperazine-1-carboxylate (0.8 g, 1.780 mmol, 1 equiv) and TEA (1.08 g, 10.680 mmol, 6 equiv) in methanol (20 mL) was added Pd(dppf)Cl₂ (0.13 g, 0.178 mmol, 0.1 equiv) in a pressure tank. The mixture was purged with nitrogen for 1 min and then was pressurized to 20 atm with carbon monoxide at 100° C. for overnight. The reaction mixture was cooled to room temperature and filtered to remove insoluble solids. The filtrate was concentrated under vacuum. The residue was purified by silica gel column chromatography, eluted with PE/EA (1:1) to afford cis-methyl 8-[(3R,5S)-4-(tert-butoxycarbonyl)-3,5-dimethylpiperazin-1-yl]-2,3-dimethylquinoxaline-5-carboxylate (0.55 g, 72%) as a solid. LCMS (ES, m/z): 429 [M+H]⁺

### Synthesis of Intermediate C184

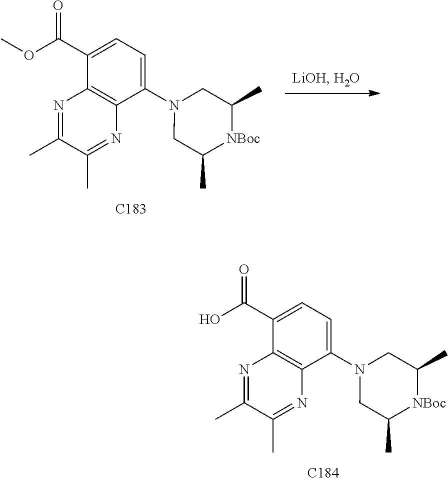

To a stirred solution of methyl cis-8-[(3R,5S)-4-(tert-butoxycarbonyl)-3,5-dimethylpiperazin-1-yl]-2,3-dimethylquinoxaline-5-carboxylate (600 mg, 1.400 mmol, 1 equiv) in THE (3 mL), methanol (3 mL) and water (3 mL) was added lithium hydroxide (268 mg, 11.200 mmol, 8 equiv) at room temperature. The resulting mixture was stirred for 16 h at 50° C. The resulting mixture was concentrated under reduced pressure. The residue was acidified to pH 6 with 1 N aq·HCl. The precipitated solids were collected by filtration and washed with water (1×10 mL), dried to afford cis-8-[(3R,5S)-4-(tert-butoxycarbonyl)-3,5-dimethylpiperazin-1-yl]-2,3-dimethylquinoxaline-5-carboxylic acid (480 mg, 83%) as a solid. LCMS (ES, m/z): 415 [M+H]⁺

### Synthesis of Intermediate C185

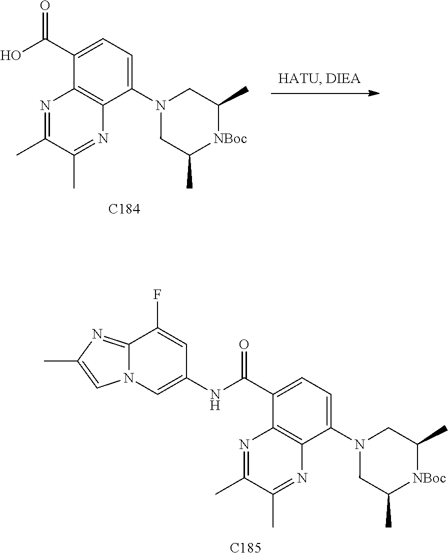

To a stirred solution of cis-8-[(3R,5S)-4-(tert-butoxycarbonyl)-3,5-dimethylpiperazin-1-yl]-2,3-dimethylquinoxaline-5-carboxylic acid (200 mg, 0.483 mmol, 1 equiv) and DIEA (124.72 mg, 0.966 mmol, 2.0 equiv) in DMF (3 mL) were added HATU (220.16 mg, 0.580 mmol, 1.2 equiv) and 8-fluoro-2-methylimidazo[1,2-a]pyridin-6-amine dihydrochloride (137.85 mg, 0.580 mmol, 1.2 equiv) at room temperature. The resulting mixture was stirred for 2 h at room temperature. The reaction was quenched with water (20 mL) at room temperature. The precipitated solids were collected by filtration and washed with water (2×10 mL), dried to afford cis-tert-butyl (2R,6S)-4-[8-({8-fluoro-2-methylimidazo[1,2-a]pyridin-6-yl}carbamoyl)-2,3-dimethylquinoxalin-5-yl]-2,6-dimethylpiperazine-1-carboxylate (226 mg, 83%) as a solid. LCMS (ES, m/z):562 [M+H]⁺

### Synthesis of Compound 318

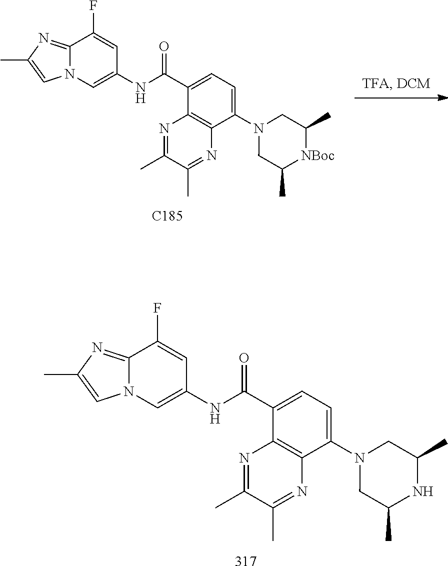

A solution of cis-tert-butyl (2R,6S)-4-[8-({8-fluoro-2-methylimidazo[1,2-a]pyridin-6-yl}carbamoyl)-2,3-dimethylquinoxalin-5-yl]-2,6-dimethylpiperazine-1-carboxylate (200 mg, 0.356 mmol, 1 equiv) and TFA (406.02 mg, 3.560 mmol, 10 equiv) in DCM (3 mL) was stirred for 2 h at room temperature. The resulting mixture was concentrated under reduced pressure. The residue was purified by reverse flash chromatography (Condition 5, Gradient 1) to afford cis-8-[(3R,5S)-3, 5-dimethylpiperazin-1-yl]-N-{8-fluoro-2-methylimidazo[1,2-a]pyridin-6-yl}-2,3-dimethylquinoxaline-5-carboxamide (35 mg, 21.30%) as a solid. LCMS (ES, m/z):462 [M+H]^{+ 1}H NMR (300 MHz, DMSO-d₆) δ 12.72 (s, 1H), 9.20 (d, J=1.6 Hz, 1H), 8.42 (d, J=8.5 Hz, 1H), 7.90 (d, J=3.4 Hz, 1H), 7.25 (dd, J=12.3, 1.7 Hz, 1H), 7.18 (d, J=8.6 Hz, 1H), 4.10 (d, J=11.2 Hz, 2H), 3.13 (s, 2H), 2.84 (s, 3H), 2.72 (s, 3H), 2.56 (d, J=10.9 Hz, 2H), 2.35 (s, 3H), 1.07 (d, J=6.2 Hz, 6H).

### Example 144: Synthesis of Compound 258

### Synthesis of Intermediate C186

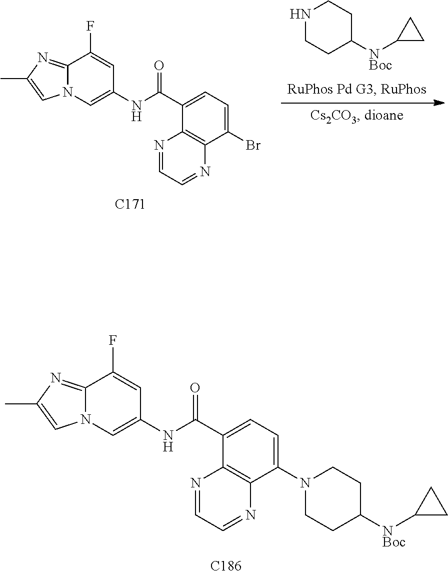

To a stirred solution of 8-bromo-N-(8-fluoro-2-methylimidazo[1,2-a]pyridin-6-yl)quinoxaline-5-carboxamide (140 mg, 0.351 mmol, 1 equiv) and tert-butyl cyclopropyl(piperidin-4-yl)carbamate (168 mg, 0.701 mmol, 2 equiv) in dioxane (10 mL) were added Cs₂CO₃ (229 mg, 0.701 mmol, 2 equiv), RuPhos (33 mg, 0.07 mmol, 0.2 equiv) and RuPhos Palladacycle Gen.3 (29 mg, 0.035 mmol, 0.1 equiv) at room temperature under nitrogen atmosphere. The resulting mixture was stirred for 3 h at 100° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl cyclopropyl(1-(8-((8-fluoro-2-methylimidazo[1,2-a]pyridin-6-yl)carbamoyl)quinoxalin-5-yl)piperidin-4-yl)carbamate (100 mg, 51%) as a solid. LCMS (ES, m/z): 560 [M+H]⁺

### Synthesis of Compound 258

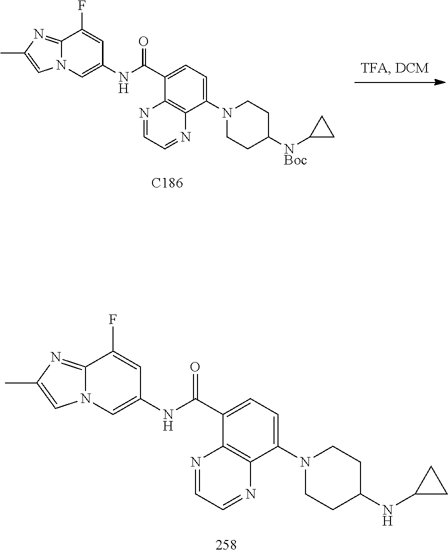

A solution of tert-butyl cyclopropyl(1-(8-((8-fluoro-2-methylimidazo[1,2-a]pyridin-6-yl)carbamoyl)quinoxalin-5-yl)piperidin-4-yl)carbamate (100 mg, 0.179 mmol, 1 equiv) in TFA (2 mL) and DCM (2 mL) was stirred for 2 h at room temperature. The resulting mixture was concentrated under vacuum. The residue was basified to pH 8 with 7M NH₃(g) in MeOH. The resulting mixture was concentrated under reduced pressure. The residue was purified by reverse flash chromatography (Condition 5, Gradient 1) to afford 8-(4-(cyclopropylamino)piperidin-1-yl)-N-(8-fluoro-2-methylimidazo[1,2-a]pyridin-6-yl)quinoxaline-5-carboxamide (40 mg, 49%) as a solid. LCMS (ES, m/z): 460 [M+H]^{+ 1}H NMR (300 MHz, DMSO-d₆) δ 12.42 (s, 1H), 9.28 (s, 1H), 9.12 (s, 1H), 9.03 (s, 1H), 8.51 (d, J=8.3 Hz, 1H), 7.92 (s, 1H), 7.46 (d, J=12.3 Hz, 1H), 7.33 (d, J=8.9 Hz, 1H), 4.07 (s, 2H), 3.11 (t, J=13.2 Hz, 2H), 2.75 (s, 1H) 2.36 (s, 3H), 2.27 (s, 1H), 2.02 (d, J=12.1 Hz, 2H), 1.57 (d, J=11.8 Hz, 2H), 0.40 (s, 2H), 0.25 (s, 2H).

### Example 145: Synthesis of Compound 259

### Synthesis of Intermediate C187

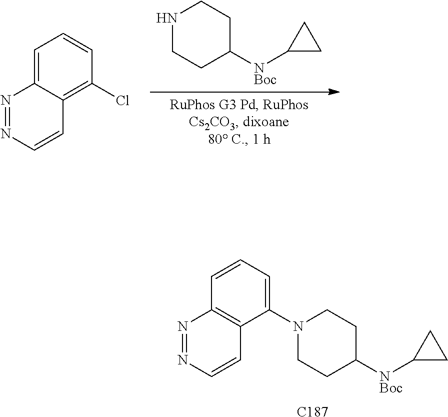 [SMILES: Clc1cccc2nnccc12 ; CC(C)(C)OC(=O)N(C1CCNCC1)C1CC1 ; "RuPhos G3 Pd, RuPhosCs2CO3, dixoane80° C., 1 h" ; CC(C)(C)OC(=O)N(C1CC1)C1CCN(c2cccc3nnccc23)CC1 ; "C187"]

To a solution of 5-chlorocinnoline (500 mg, 3.038 mmol, 1 equiv) in dioxane (5 mL) were added tert-butyl N-cyclopropyl-N-(piperidin-4-yl) carbamate (1095.21 mg, 4.557 mmol, 1.5 equiv), Cs₂CO₃ (2474.4 mg, 7.595 mmol, 2.5 equiv), RuPhos (283.5 mg, 0.608 mmol, 0.2 equiv) and RuPhos Palladacycle Gen.3 (254.0 mg, 0.304 mmol, 0.1 equiv) under nitrogen atmosphere. The reaction was stirred for 1 h at 80° C. The resulting mixture was concentrated under vacuum. The residue was purified by silica gel column chromatography, eluted with PE/EA (40%) to afford tert-butyl N-[1-(cinnolin-5-yl) piperidin-4-yl]-N-cyclopropylcarbamate (1.1 g, 98%) as an oil.

LCMS (ES, m/z): 369 [M+H]⁺

### Synthesis of Intermediate C188

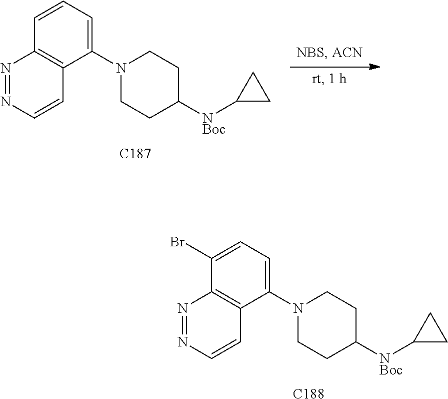

To a solution of tert-butyl N-[1-(cinnolin-5-yl) piperidin-4-yl]-N-cyclopropylcarbamate (650 mg, 1.764 mmol, 1 equiv) in MeCN (7 mL) was added NBS (313.97 mg, 1.764 mmol, 1 equiv). The mixture was stirred for 1 h at room temperature to give a brown solution. The residue was diluted with water (30 mL) and extracted with EtOAc (2×30 mL). The combined organic layers was washed with water (60 mL), brine (60 mL) and dried over anhydrous Na₂SO₄. After filtration, the filtrate was concentrated under reduced pressure. The residue was purified by silica gel column chromatography, eluted with PE/EA (1/2) to afford tert-butyl N-[1-(8-bromocinnolin-5-yl) piperidin-4-yl]-N-cyclopropylcarbamate (450 mg, 57%) as an oil. LCMS (ES, m/z): 447 [M+H]⁺

### Synthesis of Intermediate C189

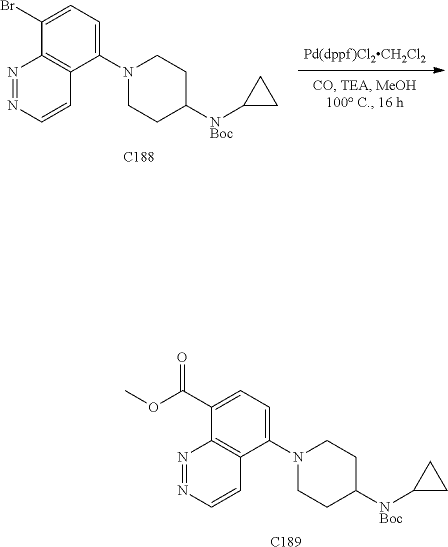 [SMILES: CC(C)(C)OC(=O)N(C1CC1)C1CCN(c2ccc(Br)c3nnccc23)CC1 ; "C188" ; COC(=O)c1ccc(N2CCC(N(C(=O)OC(C)(C)C)C3CC3)CC2)c2ccnnc12 ; "C189"]

To a solution of tert-butyl N-[1-(8-bromocinnolin-5-yl) piperidin-4-yl]-N-cyclopropylcarbamate (450 mg, 1.006 mmol, 1 equiv) in MeOH (20 mL) was added TEA (508.9 mg, 5.030 mmol, 5 equiv) and Pd(dppf)Cl₂ (73.6 mg, 0.101 mmol, 0.1 equiv). The reaction was stirred for overnight at 100° C. under CO atmosphere. The resulting mixture was concentrated under vacuum. The residue was purified by silica gel column chromatography, eluted with PE/EA (1/2) to afford methyl 5-{4-[(tert-butoxycarbonyl) (cyclopropyl)amino] piperidin-1-yl}cinnoline-8-carboxylate (430 mg, 98%) as an oil. LCMS (ES, m/z): 427 [M+H]⁺

### Synthesis of Intermediate C190

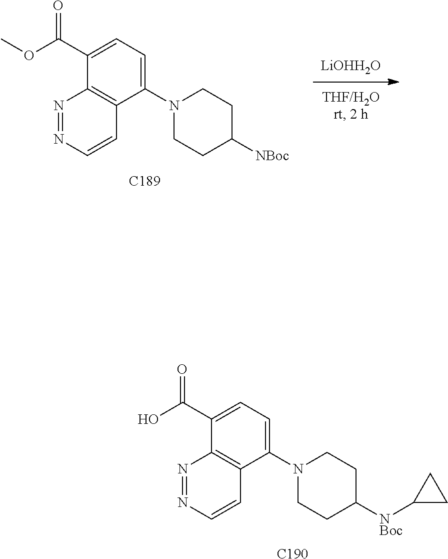

To a solution of methyl 5-{4-[(tert-butoxycarbonyl) (cyclopropyl) amino] piperidin-1-yl}cinnoline-8-carboxylate (430 mg, 1.008 mmol, 1 equiv) in THE (4 mL)/H₂O (1 mL) was added LiOH·H₂O (126.9 mg, 3.024 mmol, 3 equiv). The mixture was stirred for 2 h at room temperature. The resulting mixture was acidified to pH 5-6 with 1N HCl. The resulting mixture was extracted with EtOAc (3×30 mL), dried over anhydrous Na₂SO₄. After filtration, the filtrate was concentrated under reduced pressure to afford 5-{4-[(tert-butoxycarbonyl) (cyclopropyl) amino] piperidin-1-yl} cinnoline-8-carboxylic acid (420 mg, 99%) as an oil. LCMS (ES, m/z): 413 [M+H]⁺

### Synthesis of Intermediate C191

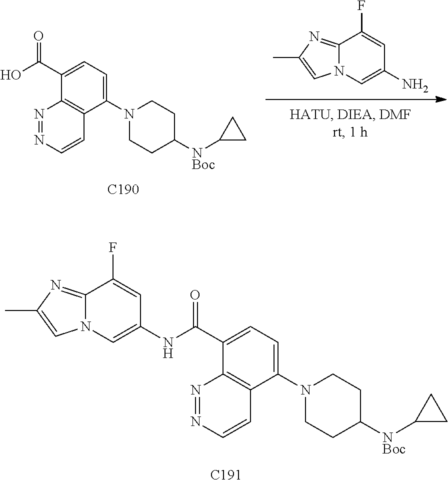 [SMILES: CC(C)(C)OC(=O)N(C1CC1)C1CCN(c2ccc(C(=O)O)c3nnccc23)CC1 ; Cc1cn2cc(N)cc(F)c2n1 ; "C190" ; Cc1cn2cc(NC(=O)c3ccc(N4CCC(N(C(=O)OC(C)(C)C)C5CC5)CC4)c4ccnnc34)cc(F)c2n1 ; "C191"]

To a mixture of 5-{4-[(tert-butoxycarbonyl) (cyclopropyl)amino] piperidin-1-yl} cinnoline-8-carboxylic acid (100 mg, 0.242 mmol, 1 equiv) in DMF (1 mL) were added 8-fluoro-2-methylimidazo[1,2-a] pyridin-6-amine (60.0 mg, 0.363 mmol, 1.5 equiv), DIEA (94.0 mg, 0.726 mmol, 3 equiv) and HATU (138.2 mg, 0.363 mmol, 1.5 equiv). The mixture was stirred at room temperature for 1 h. The resulting mixture was diluted with 20 mL of water and extracted with EtOAc (3×20 mL). The combined organic layer was washed with water (60 mL), brine (60 mL) and dried over anhydrous Na₂SO₄. After filtration, the filtrate was concentrated under reduced pressure. The residue was purified by silica gel column chromatography, eluted with PE/EA (1/5) to afford tert-butyl N-cyclopropyl-N-{1-[8-({8-fluoro-2-methylimidazo[1,2-a] pyridin-6-yl} carbamoyl) cinnolin-5-yl] piperidin-4-yl} carbamate (60 mL, 44%) as an oil. LCMS (ES, m/z): 560 [M+H]⁺

### Synthesis of Compound 259

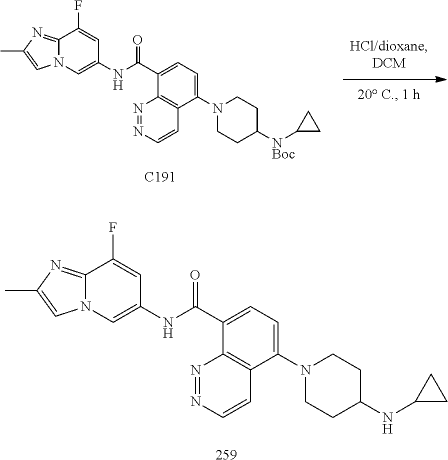

To a mixture of tert-butyl N-cyclopropyl-N-{1-[8-({8-fluoro-2-methylimidazo[1,2-a]pyridin-6-yl} carbamoyl) cinnolin-5-yl] piperidin-4-yl} carbamate (50 mg, 0.089 mmol, 1 equiv) in DCM (1 mL) was added HCl (gas) in 1,4-dioxane (0.5 mL, 4M). The mixture was stirred for 1 h at 20° C. The resulting mixture was concentrated under vacuum. The crude product was purified by Prep-HPLC (Condition 11, Gradient 1) to afford 5-[4-(cyclopropylamino) piperidin-1-yl]-N-{8-fluoro-2-methylimidazo[1,2-a] pyridin-6-yl} cinnoline-8-carboxamide (15 mg, 37%) as a solid. LCMS (ES, m/z): 460 [M+H]^{+ 1}H NMR (400 MHz, DMSO-d₆) δ 12.16 (s, 1H), 9.51 (d, J=5.9 Hz, 1H), 9.23 (d, J=1.6 Hz, 1H), 8.48 (d, J=8.0 Hz, 1H), 8.30 (d, J=5.9 Hz, 1H), 7.94 (d, J=3.1 Hz, 1H), 7.45 (d, J=8.1 Hz, 1H), 7.31 (dd, J=12.4, 1.7 Hz, 1H), 3.46 (d, J=12.1 Hz, 2H), 2.94 (t, J=11.1 Hz, 2H), 2.78 (dt, J=9.8, 5.5 Hz, 1H), 2.36-2.35 (m, 3H), 2.14 (dq, J=6.5, 3.3 Hz, 1H), 2.07 (d, J=12.5 Hz, 2H), 1.66 (q, J=10.9, 10.5 Hz, 2H), 0.41 (td, J=6.4, 4.1 Hz, 2H), 0.30-0.22 (m, 2H).

### Example 146: Synthesis of Compound 260

### Synthesis of Intermediate C192

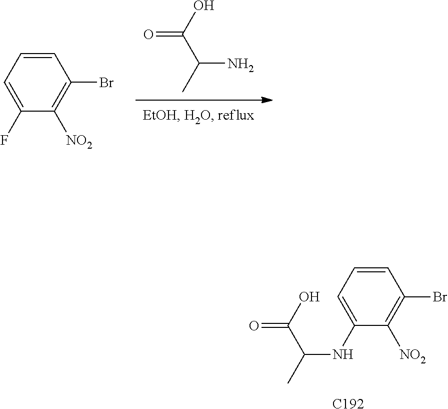

To a stirred solution of 1-bromo-3-fluoro-2-nitrobenzene (20 g, 90.910 mmol, 1 equiv) and alanine (24.3 g, 272.730 mmol, 3 equiv) in EtOH (90 mL) and H₂O (30 mL) was added Cs₂CO₃ (88.8 g, 272.730 mmol, 3equiv) at room temperature. The resulting mixture was stirred for 3 h at 70° C. The mixture was allowed to cool down to room temperature. The mixture was acidified to pH 3 with conc. HCl. The precipitated solids were collected by filtration and washed with water (3×30 mL) to afford 2-[(3-bromo-2-nitrophenyl)amino]propanoic acid (12 g, 46%) as a solid. LCMS (ES, m/z): 289 [M+H]⁺

### Synthesis of Intermediate C193

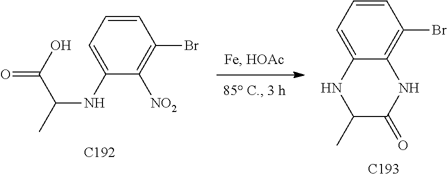 [SMILES: CC(Nc1cccc(Br)c1[N+](=O)[O-])C(=O)O ; CC1Nc2cccc(Br)c2NC1=O ; "C192" ; "C193"]

A solution of 2-[(3-bromo-2-nitrophenyl)amino]propanoic acid (12 g, 41.510 mmol, 1 equiv) and Fe (9.3 g, 166.040 mmol, 4 equiv) in HOAc (50 mL) was stirred for 3 h at 85° C. The resulting mixture was filtered; the filter cake was washed with DCM (3×100 mL). The filtrate was concentrated under reduced pressure. The residue was purified by silica gel column chromatography, eluted with PE/EA (1:1) to afford 8-bromo-3-methyl-3,4-dihydro-1H-quinoxalin-2-one (6.5 g, 65%) as a solid. LCMS (ES, m/z): 241 [M+H]⁺

### Synthesis of Intermediate C194

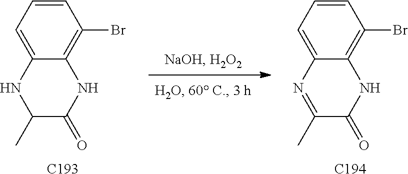

To a solution of 8-bromo-3-methyl-3,4-dihydro-1H-quinoxalin-2-one (6.5 g, 26.961 mmol, 1 equiv) in 5% aq NaOH (70 mL) was added 30% aq hydrogen peroxide (70 mL) dropwise at 0° C. The mixture was stirred at 60° C. for 3 h before it was cooled. The resulting mixture was acidified to pH 3 with conc. HCl. The precipitated solids were collected by filtration and washed with water (3×50 mL) to afford 8-bromo-3-methyl-1H-quinoxalin-2-one (4.8 g, 74%) as a solid. LCMS (ES, m/z): 239 [M+H]⁺

### Synthesis of Intermediate C195

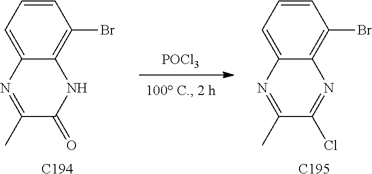

A solution of 8-bromo-3-methyl-1H-quinoxalin-2-one (6 g, 25.097 mmol, 1 equiv) in phosphorus oxychloride (50 mL) was stirred for 2 h at 100° C. The mixture was allowed to cool down to room temperature. The resulting mixture was concentrated under reduced pressure. The reaction was quenched by the addition of water/ice (100 mL) at 0° C. The mixture was neutralized to pH 7 with saturated NaHCO₃ (aq.). The resulting mixture was extracted with EtOAc (3×150 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (10:1) to afford 5-bromo-3-chloro-2-methylquinoxaline (4 g, 62%) as a solid. LCMS (ES, m/z): 258 [M+H]⁺

### Synthesis of Intermediate C196

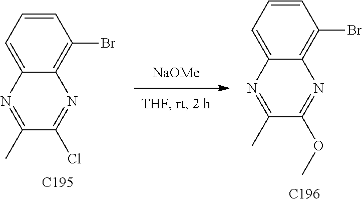

A solution of 5-bromo-3-chloro-2-methylquinoxaline (3.0 g, 11.650 mmol, 1 equiv) and sodium methoxide (1.3 g, 23.300 mmol, 2 equiv) in THF (30 mL) was stirred for 2 h at room temperature. The resulting mixture was concentrated under reduced pressure. The reaction was quenched by the addition of water (100 mL) at room temperature. The precipitated solids were collected by filtration and washed with water (3×10 mL) to afford 5-bromo-3-methoxy-2-methylquinoxaline (1.8 g, 61%) as a solid. LCMS (ES, m/z): 253 [M+H]⁺

### Synthesis of Intermediate C197

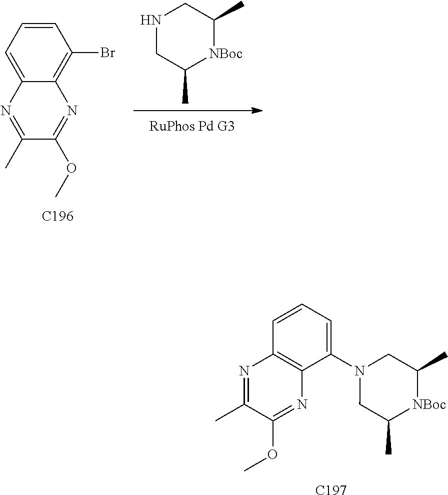

To a stirred solution of 5-bromo-3-methoxy-2-methylquinoxaline (1.8 g, 7.112 mmol, 1 equiv), Cs₂CO₃ (6.9 g, 21.336 mmol, 3 equiv) and tert-butyl (2R,6S)-2,6-dimethylpiperazine-1-carboxylate (3.1 g, 14.224 mmol, 2 equiv) in dioxane (50 mL) were added RuPhos (0.66 g, 1.422 mmol, 0.2 equiv) and RuPhos Palladacycle Gen.3 (0.59 g, 0.711 mmol, 0.1 equiv) at room temperature under nitrogen atmosphere. The resulting mixture was stirred for 2 h at 80° C. under nitrogen atmosphere. The resulting mixture was concentrated under vacuum. The residue was purified by silica gel column chromatography, eluted with PE/EA (12:1) to afford tert-butyl (2R,6S)-4-(3-methoxy-2-methylquinoxalin-5-yl)-2,6-dimethylpiperazine-1-carboxylate (1.5 g, 55%) as a solid. LCMS (ES, m/z): 387 [M+H]⁺

### Synthesis of Intermediate C198

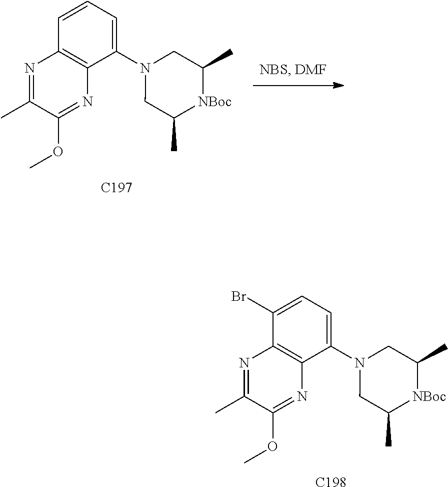

To a stirred solution of tert-butyl (2R,6S)-4-(3-methoxy-2-methylquinoxalin-5-yl)-2,6-dimethylpiperazine-1-carboxylate (1.5 g, 3.881 mmol, 1 equiv) in DMF (20 mL) was added NBS (0.69 g, 3.881 mmol, 1 equiv) in portions at room temperature. The resulting mixture was stirred for 5 h at room temperature. The reaction was quenched by the addition of water (80 mL) at room temperature. The resulting mixture was extracted with EtOAc (3×100 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (3:1) to afford tert-butyl (2R,6S)-4-(8-bromo-3-methoxy-2-methylquinoxalin-5-yl)-2,6-dimethylpiperazine-1-carboxylate (1.5 g, 83%) as a solid. LCMS (ES, m/z): 467 [M+H]⁺

### Synthesis of Intermediate C199

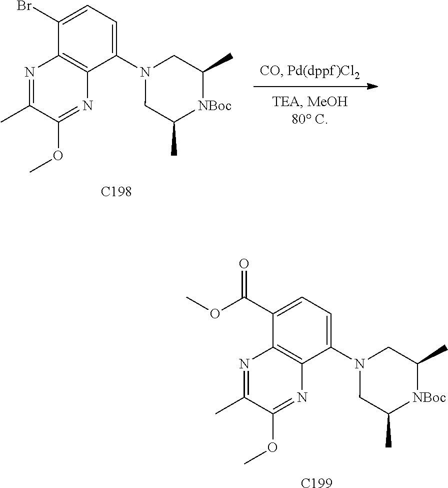

To a stirred solution of tert-butyl (2R,6S)-4-(8-bromo-3-methoxy-2-methylquinoxalin-5-yl)-2,6-dimethylpiperazine-1-carboxylate (1.5 g, 3.223 mmol, 1 equiv) and TEA (1.63 g, 16.115 mmol, 5 equiv) in MeOH (20 mL) was added Pd(dppf)Cl₂ (0.24 g, 0.322 mmol, 0.1 equiv) at room temperature. The mixture was purged with nitrogen for 1 min and then was pressurized to 20 atm with carbon monoxide at 80° C. for 3 h. The reaction mixture was cooled to room temperature and filtered to remove insoluble solids. The filtrate was concentrated under reduced pressure. The residue was purified by silica gel column chromatography, eluted with PE/EA (100:1) to afford methyl8-[(3R,5S)-4-(tert-butoxycarbonyl)-3,5-dimethylpiperazin-1-yl]-2-methoxy-3-methylquinoxaline-5-carboxylate (1.1 g, 77%) as a solid. LCMS (ES, m/z): 445 [M+H]⁺

### Synthesis of Intermediate C200

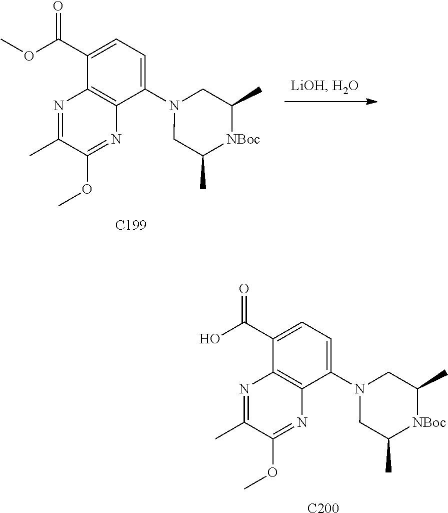

A solution of methyl 8-[(3R,5S)-4-(tert-butoxycarbonyl)-3,5-dimethylpiperazin-1-yl]-2-methoxy-3-methylquinoxaline-5-carboxylate (250 mg, 0.562 mmol, 1 equiv) and LiOH (67 mg, 2.810 mmol, 5 equiv) in MeOH (4 mL), THE (4 mL) and H₂O (2 mL) was stirred for 3 h at room temperature. The resulting mixture was concentrated under reduced pressure. The residue was acidified to pH 4 with aq·HCl (1 mol/L). The precipitated solids were collected by filtration and washed with water (3×10 mL) to afford 8-[(3R,5S)-4-(tert-butoxycarbonyl)-3,5-dimethylpiperazin-1-yl]-2-methoxy-3-methylquinoxaline-5-carboxylic acid (200 mg, 83%) as a solid. LCMS (ES, m/z): 431 [M+H]⁺

### Synthesis of Intermediate C201

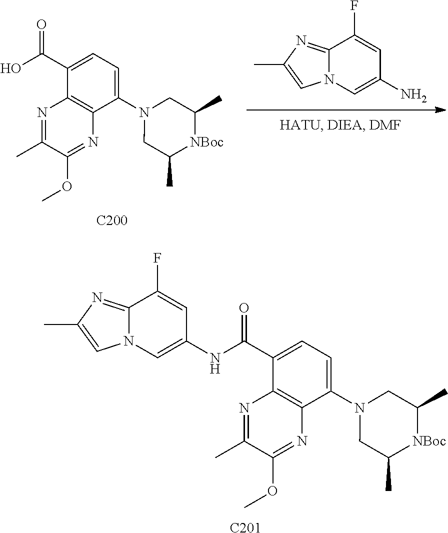

To a stirred solution of 8-[(3R,5S)-4-(tert-butoxycarbonyl)-3,5-dimethylpiperazin-1-yl]-2-methoxy-3-methylquinoxaline-5-carboxylic acid (400 mg, 0.929 mmol, 1 equiv) and 8-fluoro-2-methylimidazo[1,2-a] pyridin-6-amine hydrochloride (280 mg, 1.393 mmol, 1.5 equiv) in DMF (10 mL) were added HATU (529 mg, 1.393 mmol, 1.5 equiv) and DIEA (360 mg, 2.787 mmol, 3 equiv) at room temperature. The resulting mixture was stirred for 3 h at room temperature. The reaction was quenched by the addition of water (20 mL) at room temperature. The precipitated solids were collected by filtration and washed with water (3×10 mL) to afford tert-butyl (2R,6S)-4-[8-({8-fluoro-2-methylimidazo[1,2-a]pyridin-6-yl}carbamoyl)-3-methoxy-2-methylquinoxalin-5-yl]-2,6-dimethylpiperazine-1-carboxylate as a solid. LCMS (ES, m/z): 578 [M+H]⁺

### Synthesis of Compound 260

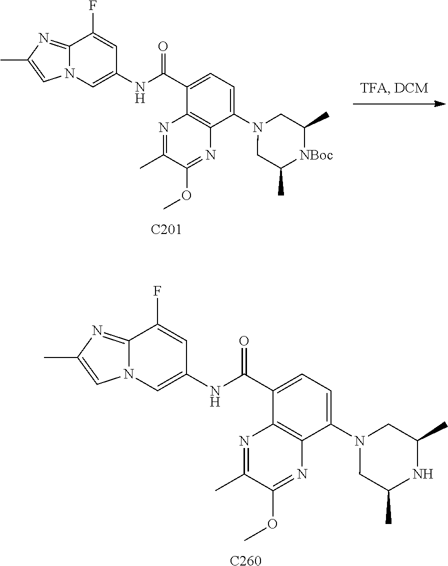

A solution of tert-butyl (2R,6S)-4-[8-({8-fluoro-2-methylimidazo[1,2-a] pyridin-6-yl}carbamoyl)-3-methoxy-2-methylquinoxalin-5-yl]-2,6-dimethylpiperazine-1-carboxylate (120 mg, 0.208 mmol, 1 equiv) and trifluoroacetic acid (2 mL) in DCM (10 mL) was stirred for 2 h at room temperature. The resulting mixture was concentrated under reduced pressure. The residue was purified by reverse flash chromatography (Condition 6, Gradient 1) to afford 8-((3R,5S)-3,5-dimethylpiperazin-1-yl)-N-(8-fluoro-2-methylimidazo[1,2-a]pyridin-6-yl)-2-methoxy-3-methylquinoxaline-5-carboxamide; 2,2,2-trifluoroacetate (20 mg, 16.26%) as a yellow solid. LCMS (ES, m/z): 478 [M+H]^{+ 1}H NMR (300 MHz, DMSO-d₆) δ 12.41 (s, 1H), 9.31 (s, 1H), 9.10 (s, 1H), 8.50 (s, 1H), 8.34 (d, J=8.4 Hz, 1H), 8.01 (s, 1H), 7.48 (d, J=12.1 Hz, 1H), 7.36 (d, J=8.5 Hz, 1H), 4.27 (d, J=12.9 Hz, 2H), 4.11 (s, 3H), 2.91 (t, J=12.1 Hz, 3H), 2.79 (s, 3H), 2.39 (s, 3H), 1.32 (d, J=6.5 Hz, 6H).

### Example 147: Synthesis of Compound 261

### Synthesis of Intermediate C202

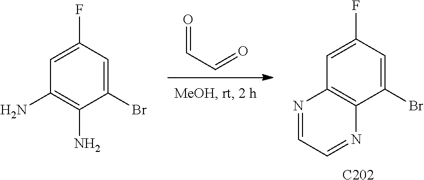

A solution of 3-bromo-5-fluorobenzene-1,2-diamine (2 g, 9.755 mmol, 1 equiv) and glyoxal (1.13 g, 19.510 mmol, 2 equiv) in methanol (30 mL) was stirred for 2 h at room temperature. The resulting mixture was concentrated under reduced pressure. The residue was diluted with EA (30 mL). The resulting mixture was washed with brine (2×20 mL), dried over anhydrous Na₂SO₄. After filtration, the filtrate was concentrated under reduced pressure to afford 5-bromo-7-fluoroquinoxaline (1.8 g, 81%) as a solid. LCMS (ES, m/z): 227 [M+H]⁺

### Synthesis of Intermediate C203

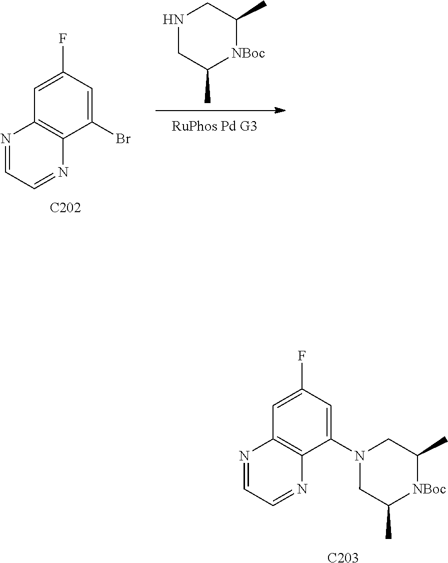 [SMILES: Fc1cc(Br)c2nccnc2c1 ; C[C@@H]1CNC[C@H](C)N1C(=O)OC(C)(C)C ; "C202" ; C[C@@H]1CN(c2cc(F)cc3nccnc23)C[C@H](C)N1C(=O)OC(C)(C)C ; "C203"]

To a solution of 5-bromo-7-fluoroquinoxaline (800 mg, 3.524 mmol, 1 equiv), cis-tert-butyl (2R,6S)-2,6-dimethylpiperazine-1-carboxylate (906 mg, 4.229 mmol, 1.2 equiv) and Cs₂CO₃ (2296 mg, 7.048 mmol, 2.0 equiv) in dioxane (10 mL) were added RuPhos (164.43 mg, 0.352 mmol, 0.1 equiv) and RuPhos Palladacycle Gen.3 (294 mg, 0.352 mmol, 0.1 equiv). After stirring for 2 h at 90° C. under a nitrogen atmosphere, the resulting mixture was concentrated under reduced pressure. The residue was purified by silica gel column chromatography, eluted with PE/EA (1:1) to afford tert-butyl (2R,6S)-4-(7-fluoroquinoxalin-5-yl)-2,6-dimethylpiperazine-1-carboxylate (600 mg, 47%) as a solid. LCMS (ES, m/z): 361 [M+H]⁺

### Synthesis of Intermediate C204

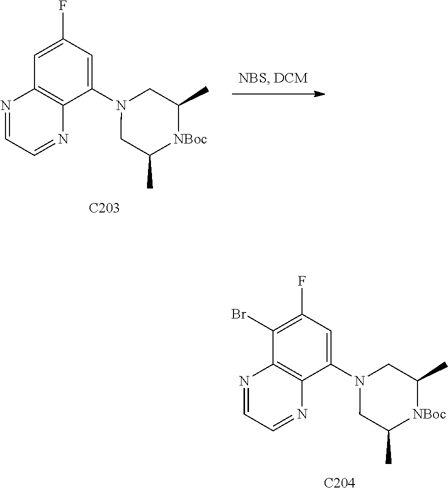 [SMILES: C[C@@H]1CN(c2cc(F)cc3nccnc23)C[C@H](C)N1C(=O)OC(C)(C)C ; "C203" ; C[C@@H]1CN(c2cc(F)c(Br)c3nccnc23)C[C@H](C)N1C(=O)OC(C)(C)C ; "C204"]

To a stirred solution of cis-tert-butyl (2R,6S)-4-(7-fluoroquinoxalin-5-yl)-2,6-dimethylpiperazine-1-carboxylate (500 mg, 1.387 mmol, 1 equiv) in DCM (20 mL) was added NBS (246 mg, 1.387 mmol, 1 equiv) in portions at 0° C. The resulting mixture was stirred for 2 h at room temperature. The reaction was quenched with water (50 mL) at room temperature. The resulting mixture was extracted with EA (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EA (3:1) to afford cis-tert-butyl (2R,6S)-4-(8-bromo-7-fluoroquinoxalin-5-yl)-2,6-dimethylpiperazine-1-carboxylate (490 mg, 80%) as a solid. LCMS (ES, m/z): 441 [M+H]⁺

### Synthesis of Intermediate C205

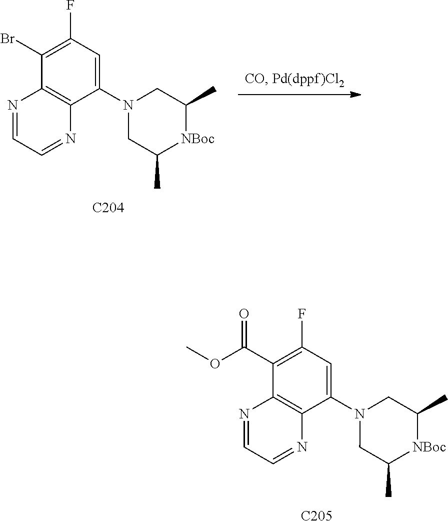

To a solution of cis-tert-butyl (2R,6S)-4-(8-bromo-7-fluoroquinoxalin-5-yl)-2,6-dimethylpiperazine-1-carboxylate (400 mg, 0.910 mmol, 1 equiv) in methanol (5 mL) were added TEA (460 mg, 4.550 mmol, 5 equiv) and Pd(dppf)Cl₂ (66 mg, 0.091 mmol, 0.1 equiv) in a pressure tank. The mixture was purged with nitrogen for 1 min and then was pressurized to 20 atm with carbon monoxide at 100° C. for 16 h. The reaction mixture was cooled to room temperature and filtered to remove insoluble solids. The filtrate was concentrated under reduced pressure. The residue was purified by silica gel column chromatography, eluted with PE/EA (5:1) to afford cis-methyl 8-[(3R,5S)-4-(tert-butoxycarbonyl)-3,5-dimethylpiperazin-1-yl]-6-fluoroquinoxaline-5-carboxylate (320 mg, 84%) as a solid. LCMS (ES, m/z): 419 [M+H]⁺

### Synthesis of Intermediate C206

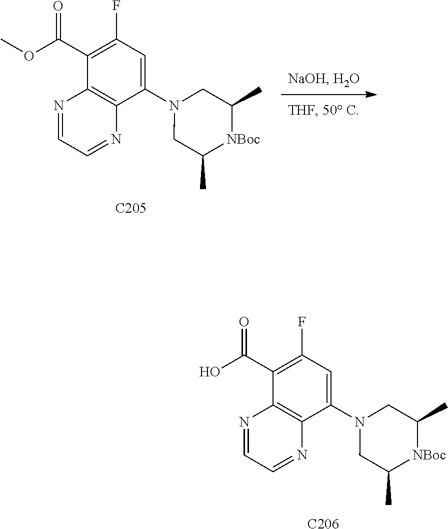

A mixture of cis-methyl 8-[(3R,5S)-4-(tert-butoxycarbonyl)-3,5-dimethylpiperazin-1-yl]-6-fluoroquinoxaline-5-carboxylate (300 mg, 0.717 mmol, 1 equiv) and NaOH (143.37 mg, 3.585 mmol, 5 equiv) in H₂O (5 mL) and THF (5 mL) was stirred for 2 h at 50° C. The resulting mixture was concentrated under reduced pressure. The residue was acidified to pH 6 with 1N aq·HCl. The precipitated solids were collected by filtration and washed with water (3×20 mL), dried to afford 8-[(3R,5S)-4-(tert-butoxycarbonyl)-3,5-dimethylpiperazin-1-yl]-6-fluoroquinoxaline-5-carboxylic acid (210 mg, 72%) as a solid. LCMS (ES, m/z): 405 [M+H]⁺

### Synthesis of Intermediate C207

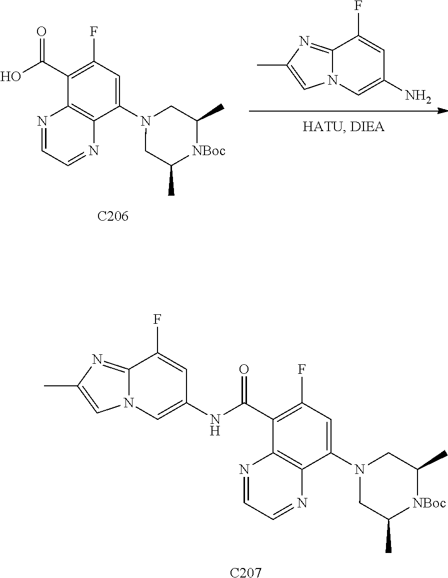

To a solution of cis-8-[(3R,5S)-4-(tert-butoxycarbonyl)-3,5-dimethylpiperazin-1-yl]-6-fluoroquinoxaline-5-carboxylic acid (200 mg, 0.495 mmol, 1 equiv) and DIEA (127.83 mg, 0.990 mmol, 2.0 equiv) in DMF (4 mL) were added HATU (225 mg, 0.594 mmol, 1.2 equiv) and 8-fluoro-2-methylimidazo[1,2-a]pyridin-6-amine (98 mg, 0.594 mmol, 1.2 equiv). After stirring for 2 h at room temperature under a nitrogen atmosphere, the resulting mixture was quenched with water (20 mL). The precipitated solids were collected by filtration and washed with water (2×10 mL), dried to afford cis-tert-butyl (2R,6S)-4-[7-fluoro-8-({8-fluoro-2-methylimidazo[1,2-a]pyridin-6-yl}carbamoyl)quinoxalin-5-yl]-2,6-dimethylpiperazine-1-carboxylate (210 mg, 77%) as a solid. LCMS (ES, m/z): 552 [M+H]⁺

### Synthesis of Compound 261

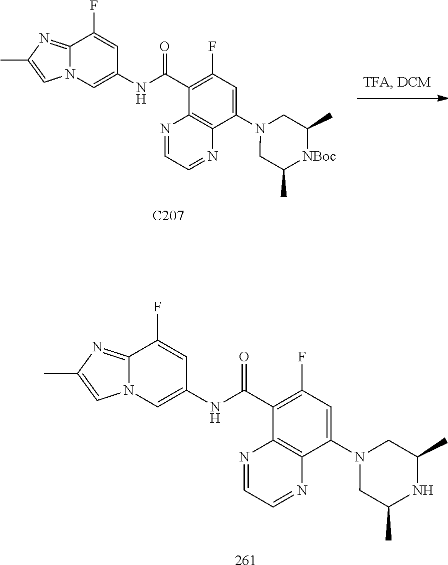

A solution of cis-tert-butyl (2R,6S)-4-[7-fluoro-8-({8-fluoro-2-methylimidazo[1,2-a]pyridin-6-yl}carbamoyl)quinoxalin-5-yl]-2,6-dimethylpiperazine-1-carboxylate (180 mg, 0.326 mmol, 1 equiv) and TFA (372.08 mg, 3.260 mmol, 10 equiv) in DCM (2 mL) was stirred for 2 h at room temperature. The resulting mixture was concentrated under reduced pressure. The residue was purified by reverse flash chromatography (Condition 5, Gradient 1) to afford cis-8-[(3R,5S)-3,5-dimethylpiperazin-1-yl]-6-fluoro-N-{8-fluoro-2-methylimidazo[1,2-a]pyridin-6-yl}quinoxaline-5-carboxamide (50 mg, 34%) as a white solid. LCMS (ES, m/z): 452 [M+H]^{+ 1}H NMR (300 MHz, DMSO-d₆) δ 10.72 (s, 1H), 9.22 (d, J=1.6 Hz, 1H), 8.97 (d, J=1.8 Hz, 1H), 8.90 (d, J=1.8 Hz, 1H), 7.95 (d, J=3.1 Hz, 1H), 7.13 (d, J=12.6 Hz, 1H), 7.06 (dd, J=12.4, 1.6 Hz, 1H), 4.00 (d, J=11.3 Hz, 2H), 3.08 (s, 2H), 2.50 (s, 2H), 2.36 (s, 3H), 1.05 (d, J=6.2 Hz, 6H).

### Example 148: Synthesis of Compound 262

### Synthesis of Intermediate C208

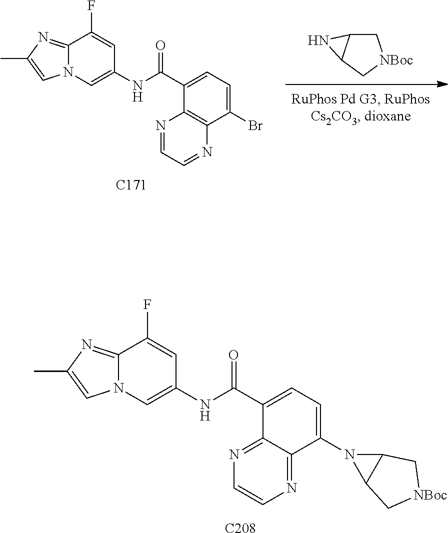 [SMILES: Cc1cn2cc(NC(=O)c3ccc(Br)c4nccnc34)cc(F)c2n1 ; CC(C)(C)OC(=O)N1CC2NC2C1 ; "C171" ; Cc1cn2cc(NC(=O)c3ccc(N4C5CN(C(=O)OC(C)(C)C)CC54)c4nccnc34)cc(F)c2n1 ; "C208"]

To a stirred solution of 8-bromo-N-{8-fluoro-2-methylimidazo[1,2-a]pyridin-6-yl}quinoxaline-5-carboxamide (100 mg, 0.250 mmol, 1 equiv) and tert-butyl 3,6-diazabicyclo[3.1.0]hexane-3-carboxylate (69 mg, 0.375 mmol, 1.5 equiv) in dioxane (5 mL) was added Cs₂CO₃ (163 mg, 0.5 mmol, 2 equiv), RuPhos (23 mg, 0.05 mmol, 0.2 equiv) and RuPhos Palladacycle Gen.3 (21 mg, 0.025 mmol, 0.1 equiv) at room temperature. The resulting mixture was stirred for 4 h at 100° C. under nitrogen atmosphere. The mixture was allowed to cool down to room temperature. The reaction was quenched with water (10 mL) at room temperature. The resulting mixture was extracted with EtOAc (3×7 mL). The combined organic layers were washed with brine (1×4 mL), dried over anhydrous Na₂SO₄. After filtration, the filtrate was concentrated under reduced pressure. The residue was purified by silica gel column chromatography, eluted with PE/EA (5:1) to afford tert-butyl 6-[8-({8-fluoro-2-methylimidazo[1,2-a]pyridin-6-yl}carbamoyl)quinoxalin-5-yl]-3,6-diazabicyclo[3.1.0]hexane-3-carboxylate (80 mg, 64%) as a solid. LCMS (ES, m/z): 504 [M+H]⁺

### Synthesis of Compound 262

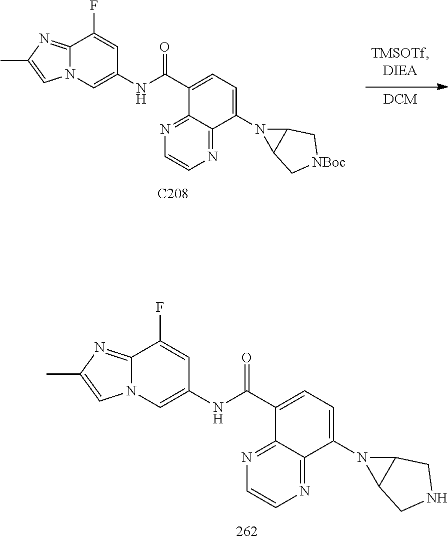

To a solution of tert-butyl 6-[8-({8-fluoro-2-methylimidazo[1,2-a]pyridin-6-yl}carbamoyl)quinoxalin-5-yl]-3,6-diazabicyclo[3.1.0]hexane-3-carboxylate (60 mg, 0.119 mmol, 1 equiv) and DIEA (46 mg, 0.357 mmol, 3 equiv) in DCM (5 mL) was added TMSOTf (80 mg, 0.357 mmol, 3 equiv) at room temperature. The resulting mixture was stirred for 1 h at room temperature. The reaction mixture was basified to pH 8 with DIEA. The resulting mixture was concentrated under reduced pressure. The residue was purified by reverse flash chromatography (Condition 5, Gradient 2) to afford 8-{3,6-diazabicyclo[3.1.0]hexan-6-yl}-N-{8-fluoro-2-methylimidazo[1,2-a]pyridin-6-yl}quinoxaline-5-carboxamide (9 mg, 19%) as a solid. LCMS (ES, m/z): 404 [M+H]^{+ 1}H NMR (300 MHz, DMSO-d₆) δ 12.36 (s, 1H), 9.28 (d, J=1.6 Hz, 1H), 9.12 (dd, J=18.9, 1.9 Hz, 2H), 8.49 (d, J=8.2 Hz, 1H), 7.93 (d, J=3.1 Hz, 1H), 7.52-7.30 (m, 2H), 3.34 (s, 2H), 3.21 (d, J=12.7 Hz, 2H), 2.72 (d, J=12.4 Hz, 2H), 2.36 (s, 3H).

### Example 149: Synthesis of Compound 264

### Synthesis of Intermediate C209

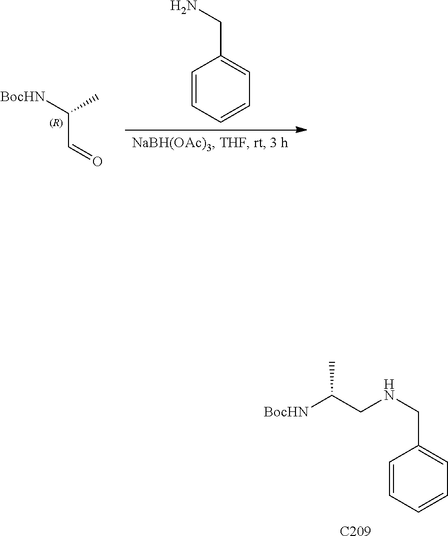

A solution of tert-butyl N-[(2R)-1-oxopropan-2-yl] carbamate (2.1 g, 12.124 mmol, 1 equiv) in THF (42 mL) was added benzylamine (1.30 g, 12.124 mmol, 1 equiv) and NaBH(OAc)₃ (5.14 g, 24.248 mmol, 2 equiv) for 3 h at room temperature. The reaction was quenched with water at room temperature. The mixture was concentrated off in vacuo. The crude product was purified by reverse phase flash (Condition 5, Gradient 2) to afford tert-butyl N-[(2R)-1-(benzylamino) propan-2-yl] carbamate (2.45 g, 76%) as an oil. LCMS (ES, m/z): 263 [M+H]⁺

### Synthesis of Intermediate C210

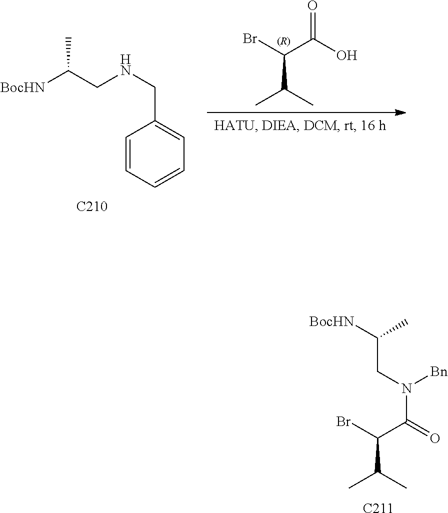

A solution of tert-butyl N-[(2R)-1-(benzylamino) propan-2-yl] carbamate (2.45 g, 9.267 mmol, 1 equiv) in DCM (50 mL) was added (2R)-2-bromo-3-methylbutanoic acid (1.68 g, 9.267 mmol, 1 equiv), DIEA (1.80 g, 13.900 mmol, 1.5 equiv) and HATU (4.23 g, 11.120 mmol, 1.2 equiv). The reaction was stirred overnight at room temperature to give a colorless solution. The residue was diluted with DCM (30 mL) and extracted with DCM (3×30 mL). The combined organic layer was dried over anhydrous Na₂SO₄. After filtration, the filtrate was concentrated under reduced pressure. The residue was purified by silica gel column chromatography, eluted with PE/EA (10/1) to afford tert-butyl N-[(2R)-1-[(2R)—N-benzyl-2-bromo-3-methylbutanamido]propan-2-yl] carbamate (3.65 g, 92%) as an oil. LCMS (ES, m/z): 427 [M+H]⁺

### Synthesis of Intermediate C212

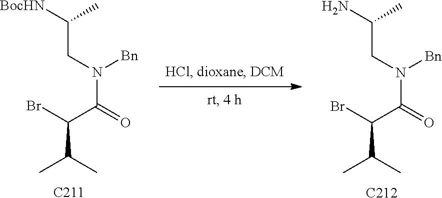 [SMILES: CC(C)[C@@H](Br)C(=O)N(Cc1ccccc1)C[C@@H](C)NC(=O)OC(C)(C)C ; "HCl, dioxane, DCM" ; "rt, 4 h" ; CC(C)[C@@H](Br)C(=O)N(Cc1ccccc1)C[C@@H](C)N ; "C211" ; "C212"]

To a stirred mixture of tert-butyl N-[(2R)-1-[(2R)—N-benzyl-2-bromo-3-methylbutanamido]propan-2-yl]carbamate (3.2 g, 7.487 mmol, 1 equiv) in DCM (60 mL) was added HCl (gas) in 1,4-dioxane (12 mL) at room temperature. The resulting mixture was stirred for 4 h at room temperature. The resulting mixture was concentrated under vacuum. This resulted in (2R)—N-[(2R)-2-aminopropyl]-N-benzyl-2-bromo-3-methylbutanamide dihydrochloride (3.0 g, 100%) as a solid. LCMS (ES, m/z): 327 [M+H]⁺

### Synthesis of Intermediate C213

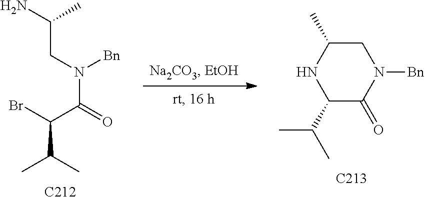

A solution of (2R)—N-[(2R)-2-aminopropyl]-N-benzyl-2-bromo-3-methylbutanamide dihydrochloride (3 g, 7.556 mmol, 1.0 equiv) in EtOH (3 mL) was added Na₂CO₃ (2.40 g, 22.670 mmol, 3 equiv). The mixture was stirred for overnight at room temperature. The resulting mixture was filtered and the filtrate was concentrated under reduced pressure. The residue was purified by silica gel column chromatography, eluted with PE/EA (3/2) to afford (3S,5R)-1-benzyl-3-isopropyl-5-methylpiperazin-2-one (720 mg, 32%) as an oil LCMS (ES, m/z): 247 [M+H]⁺

### Synthesis of Intermediate C214

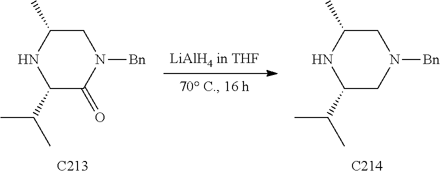 [SMILES: CC(C)[C@@H]1N[C@H](C)CN(Cc2ccccc2)C1=O ; "70° C., 16 h" ; "LiAlH4 in THF" ; CC(C)[C@H]1CN(Cc2ccccc2)C[C@@H](C)N1 ; "C213" ; "C214"]

A solution of (3S,5R)-1-benzyl-3-isopropyl-5-methylpiperazin-2-one (670 mg, 2.720 mmol, 1 equiv) in THE (7 mL) was added LiAlH₄ (309.64 mg, 8.160 mmol, 3 equiv) under nitrogen atmosphere at 0° C. The mixture was stirred overnight at 70° C. The reaction was monitored by LCMS. The reaction was quenched by the addition of water (0.31 mL), NaOH (0.31 mL, 15% w/w, aq) and water (0.93 mL). The resulting mixture was filtered, the filtrate was concentrated under reduced pressure to give (3S,5R)-1-benzyl-3-isopropyl-5-methylpiperazine (490 mg) as an oil. LCMS (ES, m/z): 233 [M+H]⁺

### Synthesis of Intermediate C215

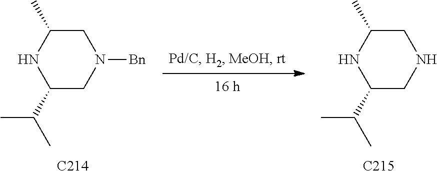

A solution of (3S,5R)-1-benzyl-3-isopropyl-5-methylpiperazine (490 mg, 2.109 mmol, 1 equiv) in MeOH (30 mL) was added Pd/C (100 mg, 10%). The mixture was stirred for 16 h under room temperature. The reaction was monitored by LCMS. The resulting mixture was filtered and the filtrate was concentrated under vacuum to afford (2S,6R)-2-isopropyl-6-methylpiperazine (220 mg, 73%) as a colorless oil. LCMS (ES, m/z): 143 [M+H]⁺

### Synthesis of Compound 264

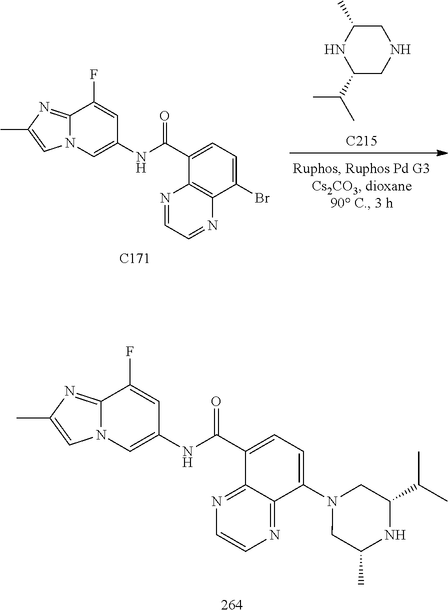

A solution of 8-bromo-N-{8-fluoro-2-methylimidazo[1,2-a] pyridin-6-yl} quinoxaline-5-carboxamide (180 mg, 0.450 mmol, 1 equiv) in 1,4-dioxane (2 mL) was added (2S,6R)-2-isopropyl-6-methylpiperazine (95.97 mg, 0.675 mmol, 1.5 equiv), Cs₂CO₃ (366.35 mg, 1.125 mmol, 2.5 equiv), RuPhos (41.98 mg, 0.090 mmol, 0.2 equiv) and RuPhos Palladacycle Gen.3 (37.62 mg, 0.045 mmol, 0.1 equiv) under nitrogen atmosphere. The reaction was stirred for 2 h at 90° C. The resulting mixture was concentrated under vacuum. The residue was purified by silica gel column chromatography, eluted with PE/EA (90%) to afford the crude product. The crude product was purified by Prep-HPLC (Condition 13, Gradient 1) to afford N-{8-fluoro-2-methylimidazo[1,2-a] pyridin-6-yl}-8-[(3S,5R)-3-isopropyl-5-methylpiperazin-1-yl]quinoxaline-5-carboxamide 2,2,2-trifluoroacetate (30 mg, 12%) as a solid. LCMS (ES, m/z): 462 [M+H]^{+ 1}H NMR (400 MHz, DMSO-d₆) δ 12.49 (s, 1H), 9.43 (s, 1H), 9.17 (d, J=1.8 Hz, 1H), 9.07 (d, J=1.8 Hz, 1H), 8.79 (s, 1H), 8.53 (t, J=14.2 Hz, 2H), 8.08 (s, 1H), 7.75 (d, J=12.1 Hz, 1H), 7.49 (d, J=8.5 Hz, 1H), 4.48 (d, J=13.1 Hz, 1H), 4.21 (d, J=12.8 Hz, 1H), 3.63 (s, 1H), 3.40 (d, J=9.0 Hz, 1H), 3.06 (dt, J=21.3, 12.1 Hz, 2H), 2.42 (s, 3H), 1.97 (h, J=6.8 Hz, 1H), 1.36 (d, J=6.4 Hz, 3H), 1.11 (d, J=6.8 Hz, 3H), 1.05 (d, J=6.8 Hz, 3H).

### Example 150: Synthesis of Compound 265, 266, and 267

### Synthesis of Compound 265

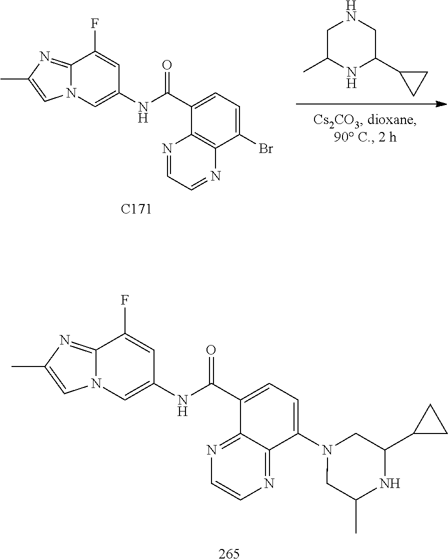

To a stirred solution of 8-bromo-N-{8-fluoro-2-methylimidazo[1,2-a]pyridin-6-yl}quinoxaline-5-carboxamide (120 mg, 0.300 mmol, 1.0 equiv) and 2-cyclopropyl-6-methylpiperazine (63.1 mg, 0.450 mmol, 1.5 equiv) in dioxane (2.4 mL) were added Cs₂CO₃ (488.5 mg, 1.500 mmol, 5.0 equiv) and Ruphos (28.0 mg, 0.060 mmol, 0.2 equiv) and RuPhos Palladacycle Gen.3 (25.1 mg, 0.030 mmol, 0.1 equiv) at room temperature under nitrogen atmosphere. The resulting mixture was stirred for 2 h at 90° C. under nitrogen atmosphere. The resulting mixture was diluted with water (5 mL). The resulting mixture was extracted with EtOAc (3×5 mL). The combined organic layers were washed with water (3×5 mL), dried over anhydrous Na₂SO₄. After filtration, the filtrate was concentrated under reduced pressure. The residue was purified by silica gel column chromatography, eluted with CH₂Cl₂/MeOH (10:1) to afford 8-(3-cyclopropyl-5-methylpiperazin-1-yl)-N-(8-fluoro-2-methylimidazo[1,2-a]pyridin-6-yl)quinoxaline-5-carboxamide (130 mg, 90%) as a solid. LCMS (ES, m/z): 460 [M+H]⁺

### Synthesis of Compound 266

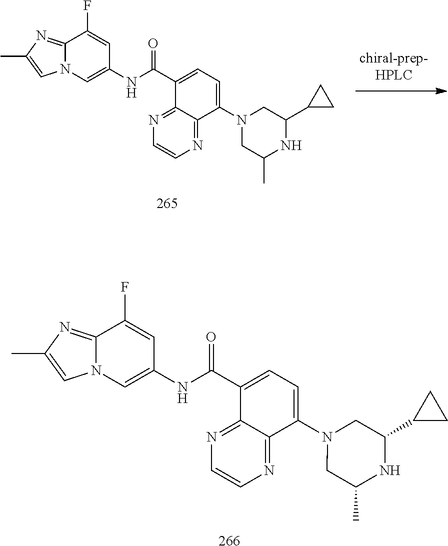 [SMILES: Cc1cn2cc(NC(=O)c3ccc(N4CC(C)NC(C5CC5)C4)c4nccnc34)cc(F)c2n1 ; "265" ; Cc1cn2cc(NC(=O)c3ccc(N4C[C@@H](C)N[C@@H](C5CC5)C4)c4nccnc34)cc(F)c2n1 ; "266"]

130 mg of 8-(3-cyclopropyl-5-methylpiperazin-1-yl)-N-(8-fluoro-2-methylimidazo[1,2-a]pyridin-6-yl)quinoxaline-5-carboxamide was purified by chiral-prep-HPLC (Condition 4, Gradient 1) to afford 8-((3S,5R)-3-cyclopropyl-5-methylpiperazin-1-yl)-N-(8-fluoro-2-methylimidazo[1,2-a]pyridin-6-yl)quinoxaline-5-carboxamide (41.6 mg, 32%) as a solid. LCMS (ES, m/z): 460 [M+H]^{+ 1}H NMR (400 MHz, DMSO-d₆) δ 12.43 (s, 1H), 9.26 (d, J=1.6 Hz, 1H), 9.10 (d, J=1.8 Hz, 1H), 9.02 (d, J=1.8 Hz, 1H), 8.51 (d, J=8.5 Hz, 1H), 7.91 (d, J=3.1 Hz, 1H), 7.45 (dd, J=12.5, 1.7 Hz, 1H), 7.31 (d, J=8.6 Hz, 1H), 4.09 (dd, J=34.2, 11.5 Hz, 2H), 3.02 (q, J=6.2, 5.3 Hz, 1H), 2.76 (t, J=11.0 Hz, 1H), 2.57 (d, J=11.1 Hz, 1H), 2.35 (s, 3H), 2.28-2.18 (m, 1H), 1.06 (d, J=6.2 Hz, 3H), 0.75 (tq, J=8.4, 4.9, 4.3 Hz, 1H), 0.48-0.38 (m, 2H), 0.31 (qd, J=10.0, 9.2, 6.0 Hz, 2H).

### Synthesis of Compound 267

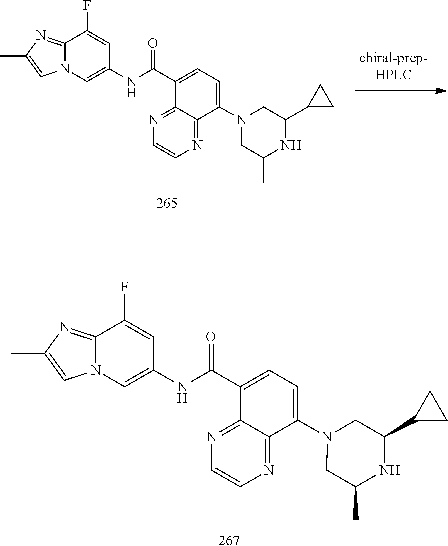 [SMILES: Cc1cn2cc(NC(=O)c3ccc(N4CC(C)NC(C5CC5)C4)c4nccnc34)cc(F)c2n1 ; "265" ; Cc1cn2cc(NC(=O)c3ccc(N4C[C@@H](C5CC5)N[C@@H](C)C4)c4nccnc34)cc(F)c2n1 ; "267"]

130 mg of 8-(3-cyclopropyl-5-methylpiperazin-1-yl)-N-(8-fluoro-2-methylimidazo[1,2-a]pyridin-6-yl)quinoxaline-5-carboxamide was purified by chiral-prep-HPLC (Condition 4, Gradient 1) to yield 8-((3R,5S)-3-cyclopropyl-5-methylpiperazin-1-yl)-N-(8-fluoro-2-methylimidazo[1,2-a]pyridin-6-yl)quinoxaline-5-carboxamide (45.8 mg, 35.23%) as a solid. LCMS (ES, m/z): 460 [M+H]^{+ 1}H NMR (400 MHz, DMSO-d₆) δ 12.43 (s, 1H), 9.26 (d, J=1.6 Hz, 1H), 9.10 (d, J=1.8 Hz, 1H), 9.02 (d, J=1.8 Hz, 1H), 8.51 (d, J=8.5 Hz, 1H), 7.91 (d, J=3.1 Hz, 1H), 7.45 (dd, J=12.5, 1.7 Hz, 1H), 7.31 (d, J=8.6 Hz, 1H), 4.09 (dd, J=34.2, 11.5 Hz, 2H), 3.02 (q, J=6.2, 5.3 Hz, 1H), 2.76 (t, J=11.0 Hz, 1H), 2.57 (d, J=11.1 Hz, 1H), 2.35 (s, 3H), 2.28-2.18 (m, 1H), 1.06 (d, J=6.2 Hz, 3H), 0.75 (tq, J=8.4, 4.9, 4.3 Hz, 1H), 0.48-0.38 (m, 2H), 0.31 (qd, J=10.0, 9.2, 6.0 Hz, 2H).

### Example 151: Synthesis of Compound 268

### Synthesis of Intermediate C216

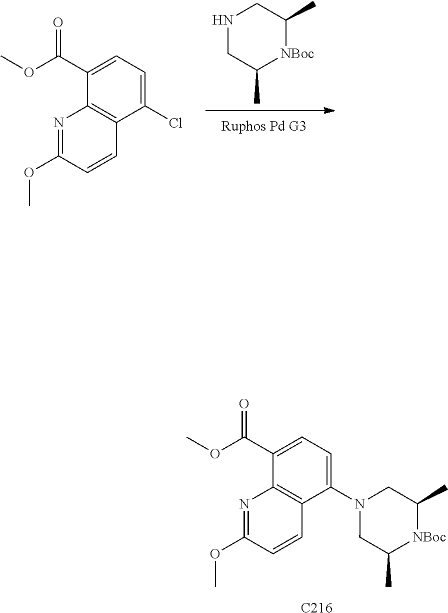

To a solution of methyl 5-chloro-2-methoxyquinoline-8-carboxylate (500 mg, 1.987 mmol, 1 equiv) and cis-tert-butyl (2R,6S)-2,6-dimethylpiperazine-1-carboxylate (851 mg, 3.974 mmol, 2.0 equiv) in dioxane (10 mL) were added Cs₂CO₃ (1941 mg, 5.961 mmol, 3.0 equiv), RuPhos (185.4 mg, 0.397 mmol, 0.2 equiv) and RuPhos Palladacycle Gen.3 (166.1 mg, 0.199 mmol, 0.1 equiv). After stirring for 1 h at 85° C. under a nitrogen atmosphere, the resulting mixture was concentrated under reduced pressure. The residue was purified by reverse flash chromatography (Condition 7, Gradient 1) to afford cis-methyl 5-[(3R,5S)-4-(tert-butoxycarbonyl)-3,5-dimethylpiperazin-1-yl]-2-methoxyquinoline-8-carboxylate (696 mg, 82%) as a solid. LCMS (ES, m/z): 430 [M−H]⁻

### Synthesis of Intermediate C217

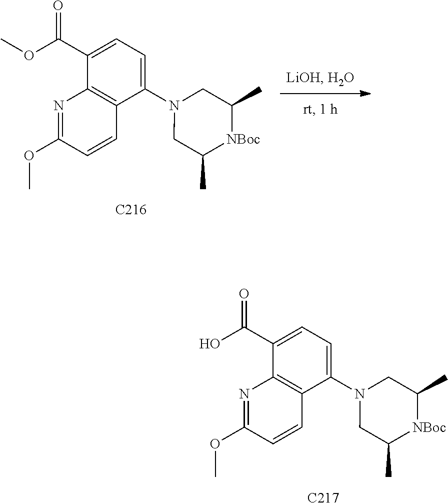

A solution of cis-methyl 5-[(3R,5S)-4-(tert-butoxycarbonyl)-3,5-dimethylpiperazin-1-yl]-2-methoxyquinoline-8-carboxylate (700 mg, 1.630 mmol, 1 equiv) in MeOH (3 mL), H₂O (3 mL) and THE (3 mL) was treated with LiOH·H₂O (410.3 mg, 9.780 mmol, 6.0 equiv) for 1 h at room temperature. The mixture was acidified to pH 2 with 2 M HCl. The precipitated solids were collected by filtration and washed with H₂O (2×10 mL) to afford cis-5-((3R,5S)-4-(tert-butoxycarbonyl)-3,5-dimethylpiperazin-1-yl)-2-methoxyquinoline-8-carboxylic acid (640 mg, 95%) as a solid. LCMS (ES, m/z): 416 [M−H]⁻

### Synthesis of Intermediate C218

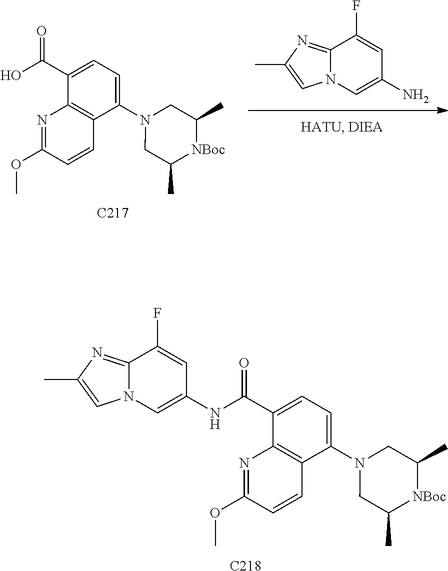

To a stirred solution of cis-5-[(3R,5S)-4-(tert-butoxycarbonyl)-3,5-dimethylpiperazin-1-yl]-2-methoxyquinoline-8-carboxylic acid (200 mg, 0.481 mmol, 1 equiv) and HATU (219.6 mg, 0.577 mmol, 1.2 equiv) in DMF (4 mL) were added DIEA (187 mg, 1.443 mmol, 3.0 equiv) and 8-fluoro-2-methylimidazo[1,2-a]pyridin-6-amine (95.4 mg, 0.577 mmol, 1.2 equiv) at room temperature. The resulting mixture was stirred for 1 h at room temperature. The resulting mixture was diluted with H₂O (10 mL). The precipitated solids were collected by filtration and washed with H₂O (3×5 mL) to afford cis-tert-butyl (2R,6S)-4-[8-({8-fluoro-2-methylimidazo[1,2-a]pyridin-6-yl}carbamoyl)-2-methoxyquinolin-5-yl]-2,6-dimethylpiperazine-1-carboxylate (210 mg, 77%) as a solid. LCMS (ES, m/z): 563 [M+H]⁺

### Synthesis of Compound 268

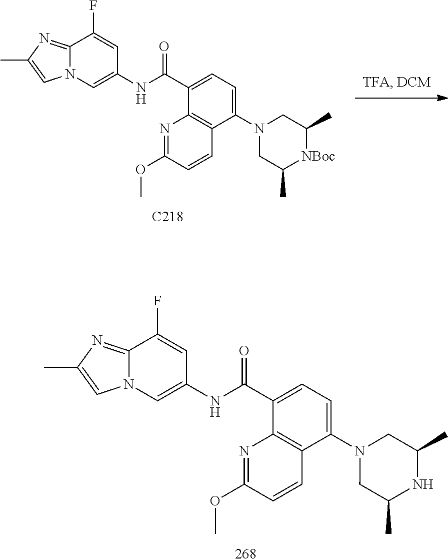

A solution of cis-tert-butyl (2R,6S)-4-[8-({8-fluoro-2-methylimidazo[1,2-a]pyridin-6-yl}carbamoyl)-2-methoxyquinolin-5-yl]-2,6-dimethylpiperazine-1-carboxylate (100 mg, 0.178 mmol, 1 equiv) and TFA (0.4 mL) in DCM (4 mL) was stirred for 2 h at room temperature. The mixture was basified to pH 8 with NH₃(g) in MeOH. The resulting mixture was concentrated under reduced pressure. The residue was purified by reverse flash chromatography (Condition 6, Gradient 1) to afford 5-[(3R,5S)-3,5-dimethylpiperazin-1-yl]-N-{8-fluoro-2-methylimidazo[1,2-a]pyridin-6-yl}-2-methoxyquinoline-8-carboxamide (50 mg, 61%) as a solid. LCMS (ES, m/z): 463 [M+H]⁺ 1H NMR (300 MHz, DMSO-d₆) δ 12.16 (s, 1H), 9.19 (d, J=1.7 Hz, 1H), 8.47 (dd, J=8.8, 3.9 Hz, 2H), 7.92 (d, J=3.0 Hz, 1H), 7.27 (d, J=11.8 Hz, 1H), 7.17 (dd, J=8.8, 3.4 Hz, 2H), 4.18 (s, 3H), 3.21 (d, J=11.0 Hz, 2H), 3.10 (s, 2H), 2.38 (d, J=10.9 Hz, 5H), 1.02 (d, J=6.2 Hz, 6H).

### Example 152: Synthesis of Compound 273

### Synthesis of Intermediate C219

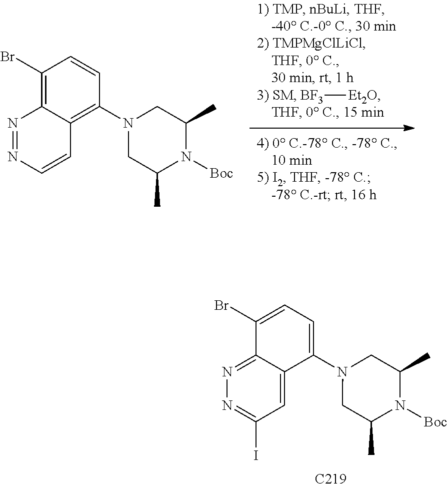

In a nitrogen flushed Schlenk flask, 2,2,6,6-tetramethylpiperidine (0.79 g, 5.594 mmol, 1.1 equiv) was dissolved in THE (10 mL). This solution was cooled to −40° C. and n-BuLi (2.24 mL, 5.594 mmol, 1.1 equiv) was added dropwise. After the addition was complete, the reaction mixture was warmed to 0° C. and stirred at this temperature for 30 min. 2,2,6,6-Tetramethylpiperidinylmagnesium chloride lithium chloride complex solution in (1M) THF (5.59 mL) was then added dropwise to the LiTMP solution. The reaction mixture was stirred at 0° C. for 30 min, warmed to room temperature, and stirred for 1 h. A dry nitrogen-flushed Schlenk flask was charged with tert-butyl (2R,6S)-4-(8-bromocinnolin-5-yl)-2,6-dimethylpiperazine-1-carboxylate (1.2 g, 2.848 mmol, 1.0 equiv) and dry THE (30 mL). The solution was cooled to 0° C., where dry BF₃.Et₂O (0.38 g, 5.594 mmol, 1.1 equiv) was added dropwise. The reaction mixture was stirred for 15 min, then cooled to −78° C., where TMP₂Mg 2LiCl was added dropwise. The reaction mixture was stirred for additional 10 min. To the above mixture was added I₂ (1.45 g, 5.714 mmol, 2.0 equiv) at −78° C. The resulting mixture was allowed warm to room temperature and stirred for additional 16 h at room temperature. The reaction was quenched by the addition of NH₄Cl (aq.) (10 mL) at 0° C. The resulting mixture was diluted with water (100 mL). The resulting mixture was extracted with EtOAc (2×100 mL). The combined organic layers were washed with Na₂SO₃ (aq.) (1×200 mL) and water (1×200 mL), dried over anhydrous Na₂SO₄. After filtration, the filtrate was concentrated under reduced pressure. The residue was purified by silica gel column chromatography, eluted with PE/EA (3:1) to afford tert-butyl (2R,6S)-4-(8-bromo-3-iodocinnolin-5-yl)-2,6-dimethylpiperazine-1-carboxylate (580 mg, 37%) as a solid. LCMS (ES, m/z): 547 [M−H]⁻

### Synthesis of Intermediate C220

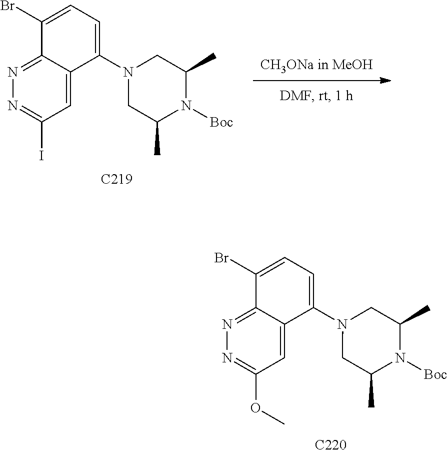 [SMILES: C[C@@H]1CN(c2ccc(Br)c3nnc(I)cc23)C[C@H](C)N1C(=O)OC(C)(C)C ; "C219" ; COc1cc2c(N3C[C@@H](C)N(C(=O)OC(C)(C)C)[C@@H](C)C3)ccc(Br)c2nn1 ; "C220"]

To a stirred mixture of tert-butyl (2R,6S)-4-(8-bromo-3-iodocinnolin-5-yl)-2,6-dimethylpiperazine-1-carboxylate (550 mg, 1.005 mmol, 1 equiv) in DMF (11 mL) were added NaOMe (362.0 mg, 2.010 mmol, 2 equiv) in MeOH (30%, w/w) dropwise at 0° C. The resulting mixture was diluted with water (20 mL). The resulting mixture was extracted with EtOAc (2×10 mL). The combined organic layers were washed with water (3×10 mL), dried over anhydrous Na₂SO₄. After filtration, the filtrate was concentrated under reduced pressure. The residue was purified by silica gel column chromatography, eluted with PE/EA (3:1) to afford tert-butyl (2R,6S)-4-(8-bromo-3-methoxycinnolin-5-yl)-2,6-dimethylpiperazine-1-carboxylate (350 mg, 77%) as an oil. LCMS (ES, m/z): 451 [M+H]⁺

### Synthesis of Intermediate C221

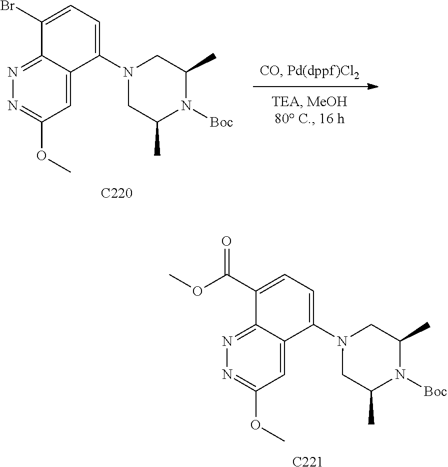 [SMILES: COc1cc2c(N3C[C@@H](C)N(C(=O)OC(C)(C)C)[C@@H](C)C3)ccc(Br)c2nn1 ; "C220" ; COC(=O)c1ccc(N2C[C@@H](C)N(C(=O)OC(C)(C)C)[C@@H](C)C2)c2cc(OC)nnc12 ; "C221"]

To a solution of tert-butyl (2R,6S)-4-(8-bromo-3-methoxycinnolin-5-yl)-2,6-dimethylpiperazine-1-carboxylate (350 mg, 0.775 mmol, 1 equiv) in 20 mL MeOH were added TEA (235.4 mg, 2.325 mmol, 3 equiv) and Pd(dppf)Cl₂ (56.7 mg, 0.078 mmol, 0.1 equiv) in a pressure tank. The mixture was purged with nitrogen for 3 min and then was pressurized to 1 Mpa with carbon monoxide at 80° C. for 16 h. The reaction mixture was cooled to room temperature and filtered to remove insoluble solids. The resulting mixture was concentrated under vacuum. The residue was purified by silica gel column chromatography, eluted with PE/EA (1:1) to afford methyl 5-[(3R,5S)-4-(tert-butoxycarbonyl)-3,5-dimethylpiperazin-1-yl]-3-methoxycinnoline-8-carboxylate (300 mg, 90%) as a solid. LCMS (ES, m/z): 431 [M+H]⁺

### Synthesis of Intermediate C222

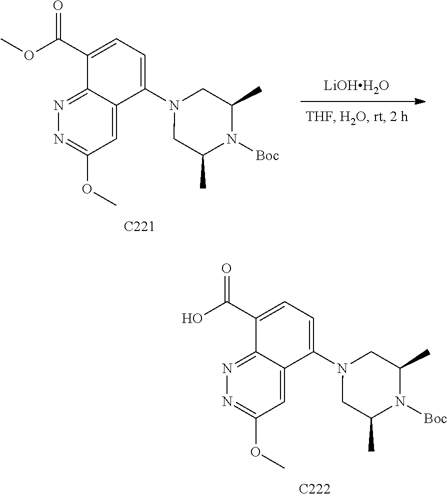 [SMILES: COC(=O)c1ccc(N2C[C@@H](C)N(C(=O)OC(C)(C)C)[C@@H](C)C2)c2cc(OC)nnc12 ; "LiOH·H2O" ; "C221" ; COc1cc2c(N3C[C@@H](C)N(C(=O)OC(C)(C)C)[C@@H](C)C3)ccc(C(=O)O)c2nn1 ; "C222"]

To a stirred solution of methyl 5-[(3R,5S)-4-(tert-butoxycarbonyl)-3,5-dimethylpiperazin-1-yl]-3-methoxycinnoline-8-carboxylate (300 mg, 0.697 mmol, 1.0 equiv) in THE (3 mL) and H₂O (3 mL) was added LiOHH₂O (146.2 mg, 3.485 mmol, 5 equiv) at room temperature. The resulting mixture was stirred for 2 h at 50° C. The mixture was acidified to pH 4 with HCl (2M).

The resulting mixture was extracted with EtOAc (3×10 mL). The combined organic layers were washed with water (3×10 mL), dried over anhydrous Na₂SO₄. After filtration, the filtrate was concentrated under reduced pressure to afford 5-[(3R,5S)-4-(tert-butoxycarbonyl)-3,5-dimethylpiperazin-1-yl]-3-methoxycinnoline-8-carboxylic acid (280 mg, 96%) as a solid. LCMS (ES, m/z): 417 [M+H]⁺

### Synthesis of Intermediate C223

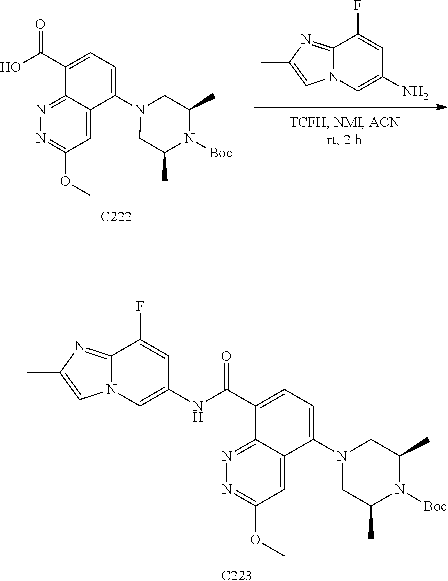 [SMILES: COc1cc2c(N3C[C@@H](C)N(C(=O)OC(C)(C)C)[C@@H](C)C3)ccc(C(=O)O)c2nn1 ; Cc1cn2cc(N)cc(F)c2n1 ; "C222" ; COc1cc2c(N3C[C@@H](C)N(C(=O)OC(C)(C)C)[C@@H](C)C3)ccc(C(=O)Nc3cc(F)c4nc(C)cn4c3)c2nn1 ; "C223"]

To a stirred mixture of 5-[(3R,5S)-4-(tert-butoxycarbonyl)-3,5-dimethylpiperazin-1-yl]-3-methoxycinnoline-8-carboxylic acid (280 mg, 0.672 mmol, 1 equiv) and 8-fluoro-2-methylimidazo[1,2-a]pyridin-6-amine (133.3 mg, 0.806 mmol, 1.2 equiv) in ACN (5.6 mL) were added NMI (220.8 mg, 2.688 mmol, 4 equiv) and TCFH (283.0 mg, 1.008 mmol, 1.5 equiv) at room temperature. The resulting mixture was stirred for 2 hr at room temperature. The precipitated solids were collected by filtration and washed with acetonitrile (2×5 mL). The residue was purified by silica gel column chromatography, eluted with CH₂Cl₂/EA (1/3) to afford tert-butyl (2R,6S)-4-[8-({8-fluoro-2-methylimidazo[1,2-a]pyridin-6-yl}carbamoyl)-3-methoxycinnolin-5-yl]-2,6-dimethylpiperazine-1-carboxylate (280 mg, 74%) as a solid. LCMS (ES, m/z): 564 [M+H]⁺

### Synthesis of Compound 273

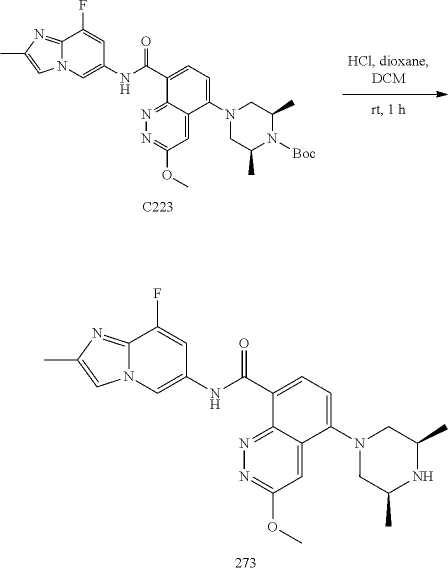

To a stirred mixture of tert-butyl (2R,6S)-4-[8-({8-fluoro-2-methylimidazo[1,2-a]pyridin-6-yl}carbamoyl)-3-methoxycinnolin-5-yl]-2,6-dimethylpiperazine-1-carboxylate (270 mg, 0.479 mmol, 1.00 equiv) in DCM (6 mL) was added HCl(gas) in 1,4-dioxane (2 mL) dropwise at 0° C. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 25% to 55% gradient in 7 min; detector, UV 254 nm) to afford 5-[(3R,5S)-3,5-dimethylpiperazin-1-yl]-N-{8-fluoro-2-methylimidazo[1,2-a]pyridin-6-yl}-3-methoxycinnoline-8-carboxamide (70 mg, 32%) as a solid. LCMS (ES, m/z): 464 [M+H]^{+ 1}H NMR (400 MHz, DMSO-d₆) δ 12.00 (s, 1H), 9.20 (d, J=1.6 Hz, 1H), 8.29 (d, J=7.9 Hz, 1H), 7.94 (d, J=3.0 Hz, 1H), 7.63 (s, 1H), 7.37-7.27 (m, 2H), 4.26 (s, 3H), 3.30-3.26 (m, 2H), 3.11-3.10 (m, 2H), 2.37 (d, J=8.4 Hz, 5H), 1.03 (d, J=6.2 Hz, 6H).

### Example 153: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I), (I), or (III)). After treatment, the levels of the HTT, MYB, or SMN2 mRNA targets were determined from each sample of cell lysate by cDNA synthesis followed by qPCR.

### Materials

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml)—used for K562/suspension cell lines
GUSB: VIC-PL, ThermoFisher 4326320E (Assay: Hs99999908_ml)—used for K562/suspension cell lines
PPIA: VIC-PL, ThermoFisher 4326316E (Assay: Hs99999904_ml)—used for A673/adherent cell lines

### Probe/Primer Sequences

### Canonical Junction (CJ)

HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/

### Alternative Junction (AJ)

HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABKFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/
AAATACTGT/ZEN/ATAGGACCTCTTCTGACATCC/3IABKFQ/

### DESCRIPTION

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3×lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 500 response in AJ increase) while the decrease in CJ mRNA levels is reported as IC₅₀ (compound concentration having 50) response in CJ decrease).

A summary of these results is illustrated in Table 4, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 4
Modulation of RNA Splicing by Exemplary Compounds

| Compound | HTT CJ | HTT AJ | MYB CJ | MYB AJ |
|---|---|---|---|---|
| No. | IC₅₀ (nM) | AC₅₀ (nM) | IC₅₀ (nM) | AC₅₀ (nM) |
| 100 | B | B | B | B |
| 105 | C | C | B | B |
| 108 | C | C | B | B |
| 109 | C | C | C | C |
| 111 | C | C | B | B |
| 113 | C | C | C | B |
| 115 | D | D | D | D |
| 116 | D | D | D | D |
| 118 | B | B | B | B |
| 119 | C | C | C | C |
| 120 | D | D | B | B |
| 121 | D | D | B | C |
| 123 | C | C | C | C |
| 125 | C | C | B | B |
| 126 | D | D | D | D |
| 127 | C | C | B | C |
| 128 | B | B | A | A |
| 129 | C | C | B | B |
| 130 | C | C | A | B |
| 131 | C | C | B | B |
| 132 | C | C | B | B |
| 133 | C | C | C | B |
| 134 | C | C | B | B |
| 137 | B | B | A | A |
| 138 | C | C | B | B |
| 140 | C | C | B | B |
| 141 | C | C | B | B |
| 146 | B | B | A | A |
| 147 | C | C | A | A |
| 148 | C | C | C | C |
| 149 | C | C | A | B |
| 150 | C | C | B | B |
| 151 | C | C | A | B |
| 152 | C | C | B | B |
| 153 | B | B | A | A |
| 154 | C | C | B | B |
| 155 | D | D | C | C |
| 156 | D | D | C | C |
| 159 | B | B | A | A |
| 161 | C | C | A | A |
| 162 | C | C | A | A |
| 163 | D | D | C | C |
| 164 | D | D | B | B |
| 165 | C | B | A | A |
| 166 | D | D | D | D |
| 167 | C | D | B | B |
| 168 | D | D | C | C |
| 169 | C | C | A | A |
| 172 | D | D | B | A |
| 173 | D | D | A | A |
| 176 | D | D | B | B |
| 177 | B | B | B | B |
| 178 | D | D | B | B |
| 179 | C | C | A | A |
| 181 | D | D | B | C |
| 182 | D | D | D | D |
| 183 | C | C | A | A |
| 185 | D | D | B | B |
| 186 | C | C | A | A |
| 187 | C | C | A | A |
| 189 | B | B | A | B |
| 190 | C | C | B | A |
| 191 | B | B | A | B |
| 192 | C | C | C | C |
| 193 | D | D | B | B |
| 194 | C | C | A | B |
| 196 | C | C | B | B |
| 197 | C | C | B | B |
| 199 | D | D | B | A |
| 200 | B | B | A | A |
| 202 | D | D | | B |
| 204 | D | D | C | D |
| 205 | B | B | A | A |
| 206 | B | B | B | B |
| 207 | B | C | A | A |
| 208 | C | C | A | A |
| 214 | B | C | A | B |
| 215 | B | C | B | B |
| 216 | C | D | B | A |
| 217 | — | — | A | A |
| 218 | D | C | A | A |
| 219 | C | C | A | A |
| 220 | C | C | A | A |
| 221 | C | C | C | C |
| 223 | B | B | A | A |
| 225 | D | D | A | B |
| 226 | D | D | C | D |
| 227 | D | D | C | C |
| 228 | B | B | A | B |
| 229 | D | D | B | A |
| 230 | C | D | D | D |
| 231 | C | C | A | A |
| 232 | C | C | B | B |
| 233 | C | C | B | B |
| 234 | C | C | B | B |
| 238 | C | C | A | A |
| 239 | C | C | A | B |
| 240 | A | B | A | B |
| 242 | D | D | B | C |
| 244 | B | B | A | A |
| 246 | C | C | B | B |
| 248 | C | D | B | B |
| 250 | D | D | B | C |
| 252 | D | D | B | B |
| 253 | C | C | A | A |
| 255 | B | B | B | B |
| 256 | C | C | C | C |
| 257 | C | C | B | C |
| 258 | B | B | B | B |
| 259 | B | B | A | A |
| 260 | B | C | B | B |
| 261 | C | C | A | A |
| 262 | B | B | B | B |
| 264 | D | D | C | B |
| 266 | C | B | A | A |
| 267 | D | D | B | B |
| 268 | C | C | B | A |
| 270 | C | C | B | A |
| 273 | B | B | B | B |
| 275 | D | D | D | D |
| 277 | B | C | A | A |
| 279 | D | D | A | B |
| 281 | D | D | C | C |
| 283 | B | B | A | B |
| 285 | C | C | B | C |
| 287 | D | D | C | D |
| 288 | C | C | A | A |
| 290 | D | D | C | D |
| 292 | D | D | C | D |
| 294 | C | D | B | B |
| 296 | B | B | A | A |
| 298 | D | D | B | B |
| 300 | B | B | A | A |
| 302 | C | C | B | B |
| 304 | C | D | A | A |
| 306 | D | D | B | B |
| 308 | D | D | C | C |
| 310 | D | D | B | B |
| 312 | C | C | A | A |
| 313 | C | C | A | A |
| 315 | D | C | B | C |
| 316 | D | C | B | C |
| 335 | C | D | B | B |
| 336 | C | C | B | B |
| 339 | C | C | B | C |
| 340 | C | C | B | B |
| 341 | C | C | B | B |
| 342 | D | D | D | D |
| 343 | C | C | C | C |
| 344 | D | D | C | D |
| 345 | C | C | B | B |
| 350 | D | D | C | C |
| 351 | C | D | B | B |
| 352 | C | C | B | B |
| 353 | D | D | D | D |
| 354 | B | B | B | B |
| 355 | D | C | B | B |
| 356 | C | D | C | C |
| 357 | D | D | D | C |
| 358 | D | D | D | D |
| 359 | D | D | B | B |
| 360 | D | D | D | D |
| 361 | C | C | B | A |
| 362 | C | C | B | B |
| 363 | C | C | B | B |
| 364 | B | C | B | B |
| 365 | B | C | B | B |
| 366 | C | C | B | B |
| 367 | C | C | C | C |
| 368 | C | C | A | A |
| 369 | C | C | B | B |
| 370 | D | D | C | D |
| 371 | B | B | B | A |
| 372 | D | D | C | C |
| 373 | D | D | B | C |
| 374 | C | C | B | B |
| 375 | C | C | B | B |
| 376 | D | D | B | B |
| 377 | C | C | A | A |
| 378 | B | B | A | A |
| 379 | C | C | B | B |
| 380 | B | B | A | A |
| 381 | D | D | B | B |
| 382 | C | C | B | A |
| 383 | C | C | B | B |
| 384 | C | C | A | A |
| 385 | C | C | A | A |
| 386 | B | B | A | A |
| 387 | C | C | A | A |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 5000 response in CJ decrease).

A summary of the results from the panel is illustrated in Tables 5A and 51B, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 5A
Modulation of RNA Splicing by Exemplary Compounds

| Compound | HTT | MYB | SMN2 | Target C |
|---|---|---|---|---|
| No. | IC₅₀ (nM) | IC₅₀ (nM) | IC₅₀ (nM) | IC₅₀ (nM) |
| 100 | B | B | B | B |
| 105 | C | B | A | D |
| 108 | C | B | A | C |
| 109 | C | C | B | |
| 111 | C | B | A | C |
| 113 | C | C | B | C |
| 115 | D | D | D | D |
| 116 | D | D | D | D |
| 118 | B | B | A | B |
| 119 | C | C | A | C |
| 120 | D | B | A | D |
| 121 | D | B | B | D |
| 123 | C | C | C | C |
| 125 | C | B | A | C |
| 126 | D | D | D | D |
| 127 | C | B | A | C |
| 128 | B | A | A | C |
| 129 | C | B | A | — |
| 130 | C | A | A | — |
| 131 | C | B | A | C |
| 132 | C | B | A | D |
| 133 | C | C | A | C |
| 134 | C | B | A | C |
| 137 | B | A | A | B |
| 138 | C | B | A | C |
| 140 | C | B | A | D |
| 141 | C | B | A | C |
| 146 | B | A | A | D |
| 147 | C | A | A | C |
| 148 | C | C | C | C |
| 149 | C | A | A | C |
| 150 | C | B | A | C |
| 151 | C | A | A | D |
| 152 | C | B | A | D |
| 153 | B | A | A | C |
| 154 | C | B | A | C |
| 155 | D | C | B | D |
| 156 | D | C | B | D |
| 159 | B | A | A | C |
| 161 | C | A | A | C |
| 162 | C | A | A | C |
| 163 | D | C | A | D |
| 164 | D | B | A | D |
| 165 | C | A | A | C |
| 166 | D | D | D | D |
| 167 | C | B | A | D |
| 168 | D | C | B | D |
| 169 | C | A | A | C |
| 172 | D | B | A | C |
| 173 | D | A | A | C |
| 176 | D | B | A | D |
| 177 | B | B | B | B |
| 178 | D | B | A | D |
| 179 | C | A | A | D |
| 181 | D | B | A | D |
| 182 | D | D | C | D |
| 183 | C | A | A | D |
| 185 | D | B | A | D |
| 186 | C | A | A | D |
| 187 | C | A | A | D |
| 189 | B | A | A | C |
| 190 | C | B | A | C |
| 191 | B | A | A | D |
| 192 | C | C | B | C |
| 193 | D | B | A | D |
| 194 | C | A | A | C |
| 196 | C | B | A | C |
| 197 | C | B | A | D |
| 199 | D | B | A | D |
| 200 | B | A | A | B |
| 202 | D | — | A | D |
| 204 | D | C | A | D |
| 205 | B | A | A | B |
| 206 | B | B | A | B |
| 207 | B | A | A | D |
| 208 | C | A | A | D |
| 214 | B | A | A | C |
| 215 | B | B | A | C |
| 216 | C | B | A | D |
| 217 | | A | A | B |
| 218 | D | A | A | D |
| 219 | C | A | A | C |
| 220 | C | A | A | C |
| 221 | C | C | A | C |
| 223 | B | A | A | B |
| 225 | D | A | A | D |
| 226 | D | C | A | D |
| 227 | D | C | A | D |
| 228 | B | A | A | B |
| 229 | D | B | A | D |
| 230 | C | D | D | D |
| 231 | C | A | A | D |
| 232 | C | B | A | C |
| 233 | C | B | A | C |
| 234 | C | B | A | C |
| 238 | C | A | A | C |
| 239 | C | A | A | C |
| 240 | A | A | A | B |
| 242 | D | B | A | D |
| 244 | B | A | A | B |
| 246 | C | B | A | D |
| 248 | C | B | A | C |
| 250 | D | B | A | D |
| 252 | D | B | A | D |
| 253 | C | A | A | D |
| 255 | B | B | B | B |
| 256 | C | C | C | C |
| 257 | C | B | A | C |
| 258 | B | B | A | B |
| 259 | B | A | A | C |
| 260 | B | B | A | C |
| 261 | C | A | A | C |
| 262 | B | B | A | B |
| 264 | D | C | A | D |
| 266 | C | A | A | D |
| 267 | D | B | A | C |
| 268 | C | B | A | C |
| 270 | C | B | A | D |
| 273 | B | B | A | B |
| 275 | D | D | D | D |
| 277 | B | A | A | C |
| 279 | D | A | A | D |
| 281 | D | C | C | D |
| 283 | B | A | A | B |
| 285 | C | B | B | C |
| 287 | D | C | C | D |
| 288 | C | A | A | C |
| 290 | D | C | D | D |
| 292 | D | C | C | D |
| 294 | C | B | A | D |
| 296 | B | A | A | C |
| 298 | D | B | A | D |
| 300 | B | A | A | B |
| 302 | C | B | A | C |
| 304 | C | A | A | D |
| 306 | D | B | A | D |
| 308 | D | C | B | D |
| 310 | D | B | A | D |
| 312 | C | A | A | D |
| 313 | C | A | A | C |
| 315 | D | B | A | D |
| 316 | D | B | A | D |
| 335 | C | B | A | D |
| 336 | C | B | A | C |
| 339 | C | B | A | C |
| 340 | C | B | A | C |
| 341 | C | B | A | C |
| 342 | D | D | B | D |
| 343 | C | C | B | C |
| 344 | D | C | B | D |
| 345 | C | B | A | C |
| 350 | D | C | B | D |
| 351 | C | B | A | C |
| 352 | C | B | A | C |
| 353 | D | D | B | D |
| 354 | B | B | B | B |
| 355 | D | B | A | D |
| 356 | C | C | B | D |
| 357 | D | D | B | D |
| 358 | D | D | D | D |
| 359 | D | B | A | D |
| 360 | D | D | C | D |
| 36 | C | B | A | C |
| 362 | C | B | A | C |
| 363 | C | B | A | C |
| 364 | B | B | A | C |
| 365 | B | B | A | C |
| 366 | C | B | A | C |
| 367 | C | C | B | C |
| 368 | C | A | A | C |
| 369 | C | B | A | C |
| 370 | D | C | B | D |
| 371 | B | B | A | C |
| 372 | D | C | B | D |
| 373 | D | B | A | D |
| 374 | C | B | A | C |
| 375 | C | B | A | C |
| 376 | D | B | A | D |
| 377 | C | A | A | D |
| 378 | B | A | A | B |
| 379 | C | B | A | D |
| 380 | B | A | A | B |
| 381 | D | B | A | D |
| 382 | C | B | A | C |
| 383 | C | B | A | C |
| 384 | C | A | A | D |
| 385 | C | A | A | D |
| 386 | B | A | A | B |
| 387 | C | A | A | C |

TABLE 5B

| Compound | HTT | MYB | SMN2 | Target C |
|---|---|---|---|---|
| No. | IC₅₀ (nM) | IC₅₀ (nM) | IC₅₀ (nM) | IC₅₀ (nM) |
| 576 | B | A | B | B |
| 577 | B | B | C | B |
| 578 | B | A | C | C |
| 579 | A | A | B | B |
| 583 | D | C | D | D |
| 589 | B | A | C | C |
| 590 | B | B | — | B |
| 591 | B | A | C | B |
| 592 | B | B | C | C |
| 597 | D | B | — | D |
| 601 | C | B | — | C |
| 602 | D | B | D | D |
| 603 | C | B | — | D |
| 604 | D | C | D | D |
| 606 | C | B | D | C |
| 607 | D | C | D | C |
| 608 | D | C | D | D |
| 609 | C | B | C | C |
| 611 | D | B | D | C |
| 612 | D | C | D | C |
| 613 | C | B | D | D |
| 614 | D | C | D | D |
| 616 | D | C | D | — |
| 617 | C | C | C | C |
| 618 | D | C | C | C |
| 619 | B | A | B | B |
| 620 | C | B | C | — |
| 621 | B | B | C | C |
| 622 | B | A | C | B |
| 624 | D | B | D | C |
| 625 | C | C | C | C |
| 637 | D | D | D | D |
| 638 | D | D | A | — |
| 639 | C | B | B | C |
| 640 | D | B | B | C |
| 641 | D | C | D | D |
| 642 | D | B | D | D |
| 643 | D | D | D | D |
| 647 | D | C | D | D |
| 648 | D | D | D | D |
| 649 | B | B | C | — |
| 650 | C | B | D | — |
| 654 | B | C | C | — |
| 655 | B | A | C | — |
| 656 | B | A | C | — |
| 657 | D | D | D | D |
| 658 | D | D | D | D |
| 659 | B | B | C | C |
| 660 | B | A | C | C |
| 661 | D | C | — | D |
| 662 | D | D | D | D |
| 663 | B | B | D | C |
| 664 | D | D | D | — |
| 665 | A | A | C | B |
| 666 | C | B | C | — |
| 667 | D | D | D | — |
| 668 | D | C | D | — |
| 669 | B | A | D | — |
| 670 | C | B | D | — |
| 671 | C | B | D | — |

### Example 154: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), or (III)). After treatment for 24 hours, the protein abundance of a specific target was determined by measuring luminescence.

### Materials

Promega Nano-Glo HiBiT Lytic Detection System (cat #N3030)
Corning 384-well TC-treated microplates (cat #3570)
Synthego Engineered Cells Knock-In Clones

TABLE 6
Design of genetically modified HiBiT cell lines

| | | | Guide | Guide | |
|---|---|---|---|---|---|
| Cell | | | RNA | RNA cut | |
| Line | Gene | Modification | Sequence | location | Donor Sequence |
| K562 | MYB | HiBiT | GCGCCA | chr6: | CGGTGCGGTCCCCGCGGCTC |
| | | | TGGCCC | 135,181,526 | TCGGCGGAGCCCCGCGCCCG |
| | | | GAAGAC | | CCGCGCCATGgtgagcggctggcgg |
| | | | CC | | ctgttcaagaagattagcGGCAGCTCC |
| | | | | | GGAGGATCTAGCGGCGCCCG |
| | | | | | AAGACCCCGGCACAGgtaacgg |
| | | | | | ggagccggggggcggccgaggg |
| K562 | HTT | HiBiT | CAGCTTT | chr4: | CGAGTCGGCCCGAGGCCTCC |
| | | | TCCAGG | 3,074,830 | GGGGACTGCCGTGCCGGGCG |
| | | | GTCGCC | | GGAGACCGCCATGgtgagcggctg |
| | | | A | | gcggctgttcaagaagattagcGGCAGC |
| | | | | | TCCGGAGGATCTAGCGGCGC |
| | | | | | GACCCTGGAAAAGCTGATGA |
| | | | | | AGGCCTTCGAGTCCCTCAAG |
| | | | | | TCCTTCCA |

### DESCRIPTION

Cells were maintained in IMDM with 10% FBS. Before the assay, cells were diluted with phenolphthalein-free growth media (IMDM+1% FBS media) and were seeded in a 384-well plate at a density of 10000 cells/well (for each cell line listed in Table 6). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 6, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 50% value to determine a concentration for protein abundance at 50% (IC₅₀) the untreated control.

A summary of the results for protein abundance is illustrated in Table 7, wherein A represents <100 nM; B represents 100-1000 nM; C represents 1000-9999 nM; and D represents greater than 10 μM.

TABLE 7

| Compound | | | Target |
|---|---|---|---|
| No. | HTT | MYB | C |
| 101 | B | A | B |
| 100 | B | B | B |
| 113 | C | C | C |
| 103 | B | A | C |
| 111 | B | A | C |
| 127 | C | B | C |
| 137 | B | A | B |
| 133 | C | B | C |
| 134 | C | A | C |
| 128 | B | A | B |
| 130 | C | A | C |
| 105 | C | B | C |
| 139 | B | A | C |
| 109 | C | C | C |
| 138 | C | B | C |
| 108 | C | B | D |
| 112 | B | A | C |
| 131 | B | A | C |
| 126 | D | D | D |
| 116 | D | D | D |
| 120 | D | B | D |
| 141 | C | A | C |
| 118 | B | A | B |
| 122 | C | C | C |
| 123 | C | C | C |
| 124 | C | A | C |
| 125 | C | B | C |
| 115 | D | D | D |
| 117 | B | A | C |
| 119 | C | B | C |
| 146 | B | A | C |
| 140 | B | A | D |
| 149 | C | A | C |
| 150 | C | B | C |
| 151 | C | A | C |
| 152 | C | A | D |
| 153 | C | A | C |
| 154 | C | A | C |
| 155 | D | D | D |
| 156 | D | C | D |
| 157 | C | A | C |
| 158 | B | B | C |
| 159 | B | A | C |
| 161 | C | A | C |
| 162 | B | A | C |
| 163 | D | C | D |
| 164 | C | B | D |
| 165 | B | A | C |
| 166 | D | D | D |
| 167 | C | B | C |
| 168 | D | C | D |
| 169 | B | A | C |
| 171 | B | A | C |
| 172 | D | A | D |
| 173 | D | A | D |
| 176 | C | B | C |
| 177 | B | B | B |
| 178 | C | B | D |
| 179 | C | A | D |
| 180 | D | D | D |
| 181 | D | C | D |
| 182 | D | D | D |
| 183 | B | A | C |
| 186 | C | A | C |
| 188 | C | A | C |
| 195 | B | A | B |
| 189 | B | A | C |
| 196 | C | A | C |
| 187 | C | A | C |
| 190 | C | B | C |
| 191 | B | A | C |
| 192 | C | C | C |
| 193 | C | A | C |
| 194 | C | A | B |
| 197 | B | A | C |
| 198 | B | B | B |
| 199 | C | A | C |
| 200 | A | A | B |
| 208 | C | A | C |
| 217 | B | A | B |
| 218 | C | A | C |
| 220 | C | A | C |
| 216 | C | A | C |
| 206 | B | A | B |
| 219 | C | A | C |
| 207 | B | A | C |
| 202 | C | A | C |
| 203 | C | C | C |
| 204 | C | B | C |
| 205 | B | A | B |
| 223 | B | A | B |
| 224 | D | A | D |
| 225 | D | A | D |
| 226 | C | B | C |
| 227 | C | B | B |
| 214 | C | A | C |
| 215 | C | A | C |
| 221 | C | B | C |
| 228 | B | A | B |
| 229 | D | A | D |
| 230 | D | D | D |
| 231 | C | A | C |
| 232 | C | A | C |
| 233 | C | A | C |
| 234 | C | A | C |
| 238 | B | A | C |
| 239 | B | A | C |
| 240 | A | A | B |
| 242 | D | B | D |
| 244 | B | A | B |
| 246 | C | B | C |
| 248 | C | B | D |
| 250 | D | B | D |
| 252 | C | B | C |
| 253 | C | A | C |
| 255 | B | B | B |
| 256 | C | C | C |
| 257 | C | C | C |
| 258 | B | A | B |
| 259 | B | A | C |
| 260 | C | A | C |
| 261 | C | A | C |
| 262 | B | B | B |
| 263 | D | B | D |
| 266 | B | A | B |
| 267 | C | B | D |
| 268 | C | A | C |
| 270 | C | B | C |
| 273 | B | B | B |
| 275 | D | D | D |
| 277 | B | A | C |
| 279 | C | A | D |
| 281 | C | C | C |
| 283 | B | A | B |
| 285 | C | B | C |
| 287 | D | C | D |
| 288 | C | A | C |
| 290 | D | D | D |
| 292 | D | C | C |
| 294 | D | B | D |
| 296 | B | A | B |
| 298 | C | B | C |
| 300 | B | A | B |
| 302 | C | B | C |
| 304 | D | A | D |
| 306 | C | B | C |
| 308 | D | C | D |
| 310 | D | C | D |
| 312 | C | A | C |
| 313 | B | A | C |
| 315 | C | B | C |
| 316 | C | B | D |
| 335 | C | B | C |
| 339 | C | B | C |
| 340 | C | B | D |
| 341 | C | B | D |
| 342 | D | D | D |
| 343 | C | C | C |
| 344 | D | C | D |
| 345 | C | B | C |
| 351 | C | B | D |
| 352 | C | B | C |
| 353 | D | D | D |
| 354 | B | B | B |
| 355 | C | B | C |
| 356 | C | C | C |
| 357 | D | C | D |
| 358 | D | D | D |
| 359 | D | B | D |
| 360 | D | D | D |
| 361 | C | B | C |
| 362 | C | B | C |
| 363 | C | A | C |
| 364 | B | B | C |
| 365 | B | B | C |
| 366 | C | B | C |
| 367 | C | C | C |
| 368 | C | A | C |
| 369 | C | B | C |
| 370 | D | C | D |
| 371 | B | B | B |
| 372 | D | C | D |
| 373 | C | B | C |
| 374 | C | B | C |
| 375 | C | B | C |
| 376 | C | B | D |
| 377 | C | B | C |
| 378 | B | A | B |

### Example 155: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials

Promega CellTiter-Glo® 2.0 Cell Viability Assay (cat #G9241)
Corning 384-well TC-treated microplates (cat #3570)

### DESCRIPTION

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I), (II), or (III)) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent.

A summary of the results for viability is illustrated in Table 8, wherein A represents <100 nM; B represents 100-1000 nM; C represents 1000-9999 nM; and D represents greater than 10 μM in K562 cells.

TABLE 8

| Compound | |
|---|---|
| No. | Viability |
| 101 | B |
| 100 | B |
| 103 | A |
| 111 | B |
| 127 | C |
| 137 | A |
| 133 | C |
| 134 | B |
| 128 | B |
| 130 | B |
| 105 | B |
| 139 | A |
| 138 | C |
| 108 | B |
| 112 | B |
| 131 | B |
| 126 | D |
| 116 | C |
| 120 | C |
| 141 | C |
| 118 | A |
| 122 | C |
| 123 | C |
| 124 | B |
| 125 | B |
| 115 | D |
| 117 | B |
| 119 | B |
| 146 | A |
| 140 | B |
| 149 | B |
| 150 | B |
| 151 | B |
| 152 | B |
| 153 | B |
| 154 | B |
| 155 | B |
| 156 | C |
| 157 | A |
| 158 | B |
| 159 | A |
| 161 | B |
| 162 | B |
| 163 | C |
| 164 | B |
| 165 | A |
| 166 | D |
| 167 | B |
| 168 | C |
| 169 | A |
| 171 | B |
| 172 | B |
| 173 | B |
| 176 | B |
| 177 | B |
| 178 | C |
| 179 | B |
| 181 | C |
| 182 | D |
| 183 | B |
| 186 | B |
| 188 | B |
| 195 | A |
| 189 | B |
| 196 | A |
| 187 | B |
| 190 | A |
| 191 | B |
| 192 | C |
| 193 | A |
| 194 | A |
| 197 | C |
| 198 | B |
| 199 | B |
| 200 | B |
| 208 | A |
| 217 | B |
| 218 | B |
| 220 | A |
| 216 | C |
| 206 | B |
| 219 | A |
| 207 | B |
| 202 | A |
| 203 | C |
| 204 | A |
| 205 | B |
| 223 | B |
| 224 | B |
| 225 | C |
| 226 | A |
| 227 | A |
| 214 | B |
| 215 | B |
| 221 | B |
| 228 | A |
| 229 | A |
| 230 | D |
| 231 | B |
| 232 | B |
| 233 | A |
| 234 | B |
| 238 | B |
| 239 | B |
| 240 | A |
| 242 | B |
| 244 | A |
| 246 | B |
| 248 | B |
| 250 | B |
| 252 | B |
| 253 | A |
| 255 | B |
| 256 | C |
| 257 | B |
| 258 | B |
| 259 | A |
| 260 | C |
| 261 | B |
| 262 | B |
| 263 | C |
| 266 | B |
| 267 | B |
| 268 | B |
| 270 | B |
| 273 | B |
| 277 | B |
| 281 | C |
| 283 | B |
| 285 | C |
| 287 | D |
| 288 | C |
| 290 | D |
| 292 | D |
| 294 | D |
| 296 | B |
| 298 | C |
| 300 | B |
| 302 | C |
| 304 | B |
| 306 | B |
| 308 | C |
| 310 | C |
| 312 | B |
| 313 | A |
| 315 | B |
| 316 | B |
| 335 | B |
| 339 | B |
| 340 | B |
| 341 | B |
| 342 | C |
| 343 | C |
| 344 | C |
| 345 | B |
| 351 | B |
| 352 | B |
| 353 | C |
| 354 | B |
| 355 | C |
| 356 | C |
| 357 | C |
| 358 | D |
| 359 | B |
| 360 | D |
| 361 | B |
| 362 | B |
| 363 | A |
| 364 | B |
| 365 | B |
| 366 | C |
| 367 | C |
| 368 | B |
| 369 | C |
| 370 | D |
| 371 | A |
| 372 | C |
| 373 | B |
| 374 | B |
| 375 | B |
| 376 | B |
| 377 | B |
| 378 | A |
| 379 | B |
| 380 | B |
| 381 | B |
| 382 | B |
| 383 | B |
| 384 | B |
| 385 | B |
| 386 | A |
| 387 | B |

### Equivalents and Scope

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 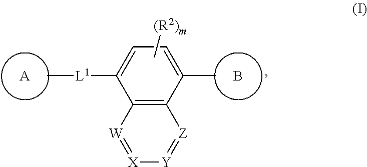
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
W, X, Y, and Z are each independently C(R^{3a}) or N, wherein at least one of W, X, Y, and Z is independently N;
L¹ is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, heteroaryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
each R⁴ is independently hydrogen, C₁-C₆-alkyl, or C₁-C₆-haloalkyl;
each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1};
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is hydrogen or C₁-C₆-alkyl;
m is 0, 1, or 2; and
x is 0, 1, or 2.

2. The compound of claim 1, wherein each of A and B is independently heteroaryl or heterocyclyl, each of which is optionally substituted with one or more R¹.

3. The compound of any one of the preceding claims, wherein one of A and B is independently a monocyclic heteroaryl or bicyclic heteroaryl, each of which is optionally substituted with one or more R¹.

4. The compound of any one of the preceding claims, wherein one of A and B is independently a bicyclic heteroaryl optionally substituted with one or more R¹.

5. The compound of any one of the preceding claims, wherein one of A and B is independently a nitrogen-containing heteroaryl optionally substituted with one or more R¹.

6. The compound of any one of the preceding claims, wherein one of A and B is a 5-10 membered heteroaryl optionally substituted with one or more R¹.

7. The compound of any one of the preceding claims, wherein one of A and B is independently selected from 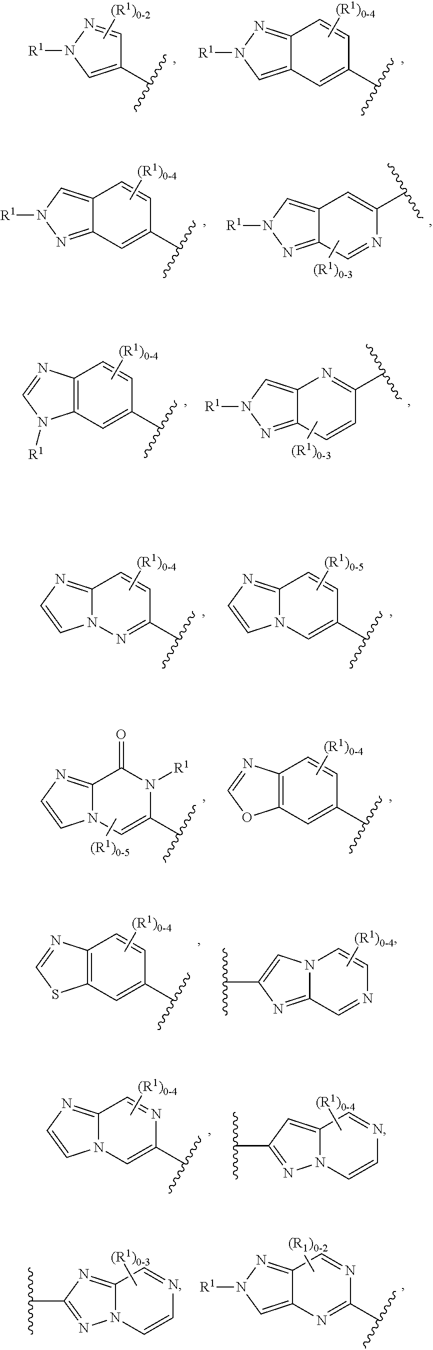 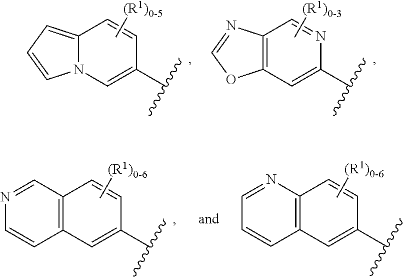
wherein R¹ is as described in claim 1.

8. The compound of any one of the preceding claims, wherein one of A and B is independently selected from 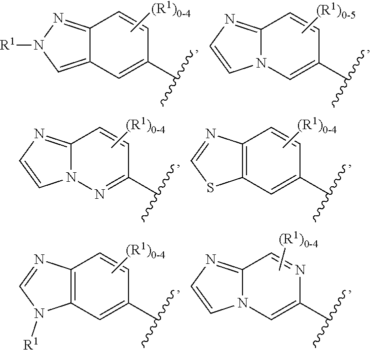
wherein R¹ is as described in claim 1.

9. The compound of any one of the preceding claims, wherein one of A and B is independently selected from N 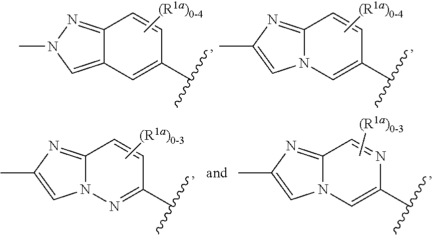
and wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

10. The compound of any one of the preceding claims, wherein one of A and B is independently 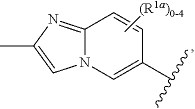
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

11. The compound of any one of the preceding claims, wherein A is selected from 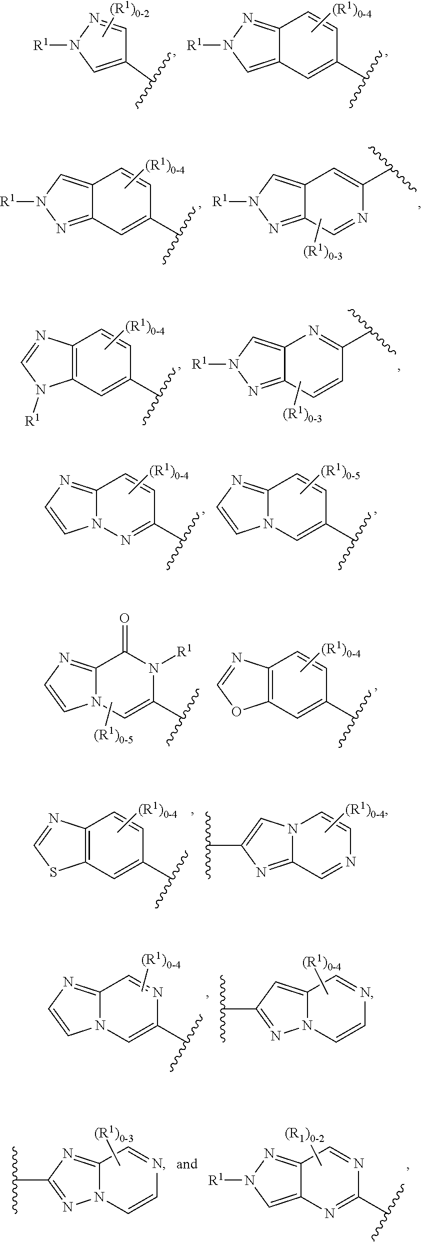
wherein R¹ is as described in claim 1, wherein R¹ is as described in claim 1.

12. The compound of any one of the preceding claims, wherein B is selected from 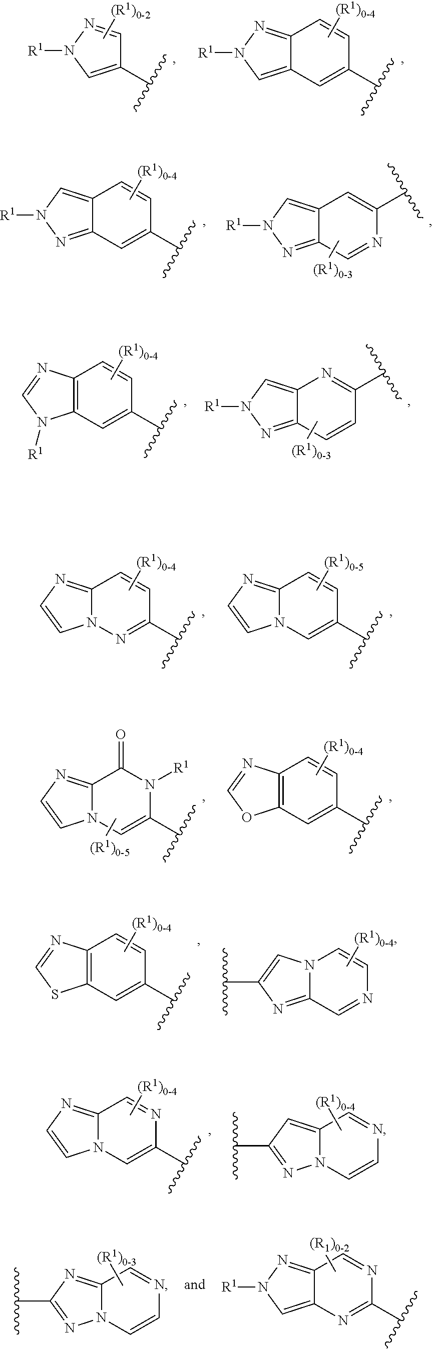
wherein R¹ is as described in claim 1.

13. The compound of any one of the preceding claims, wherein A is selected from 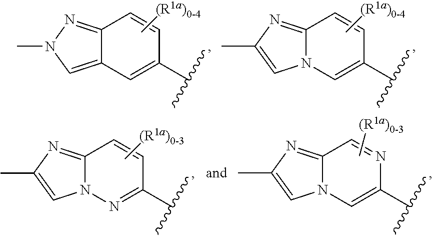
wherein each R^{a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

14. The compound of any one of the preceding claims, wherein A is 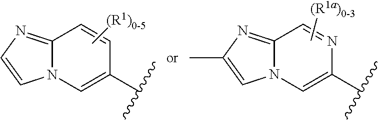
wherein R¹ is as described in claim 1.

15. The compound of any one of the preceding claims, wherein one of A and B is independently selected from

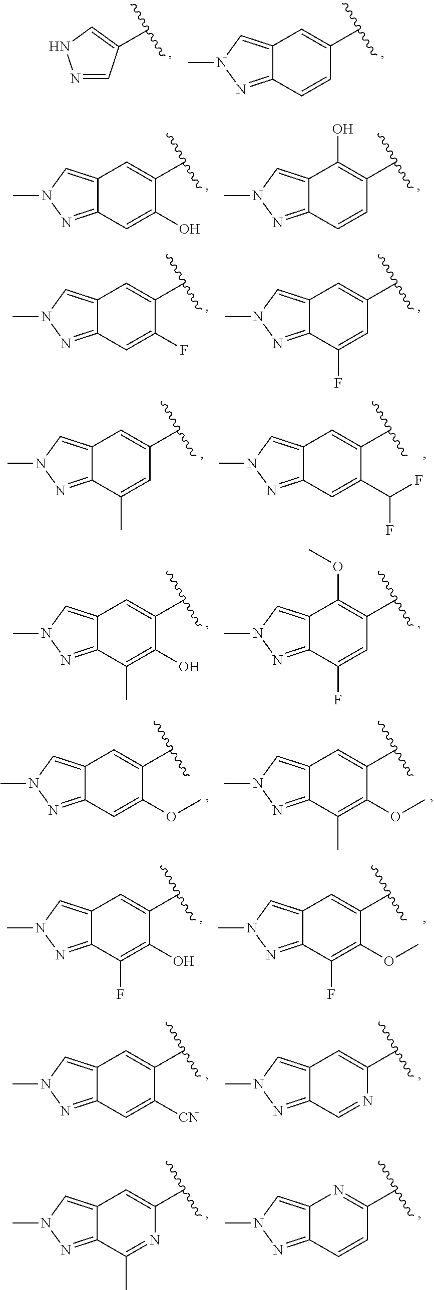

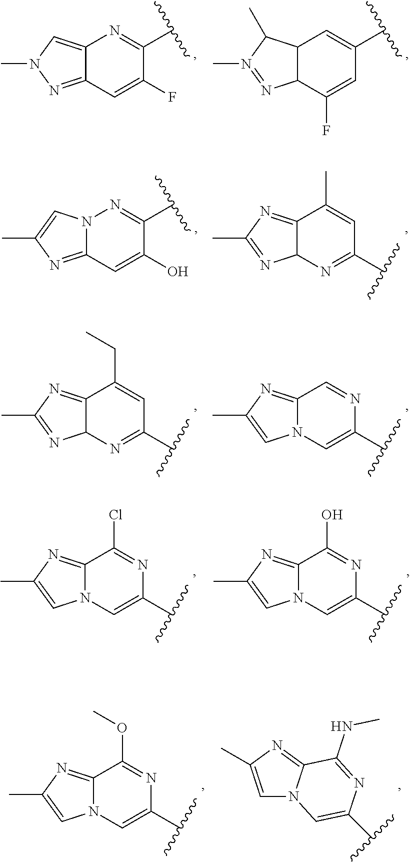

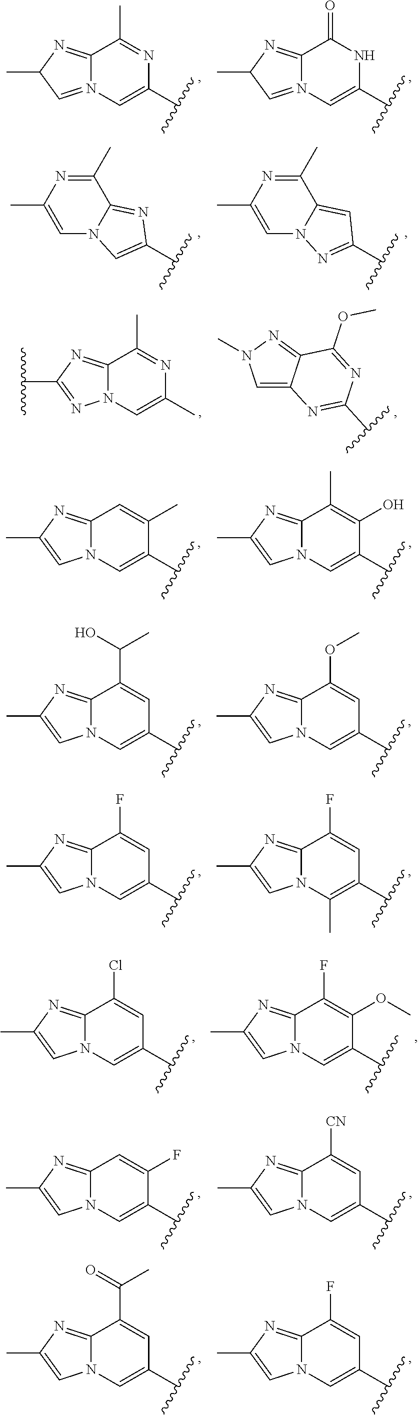

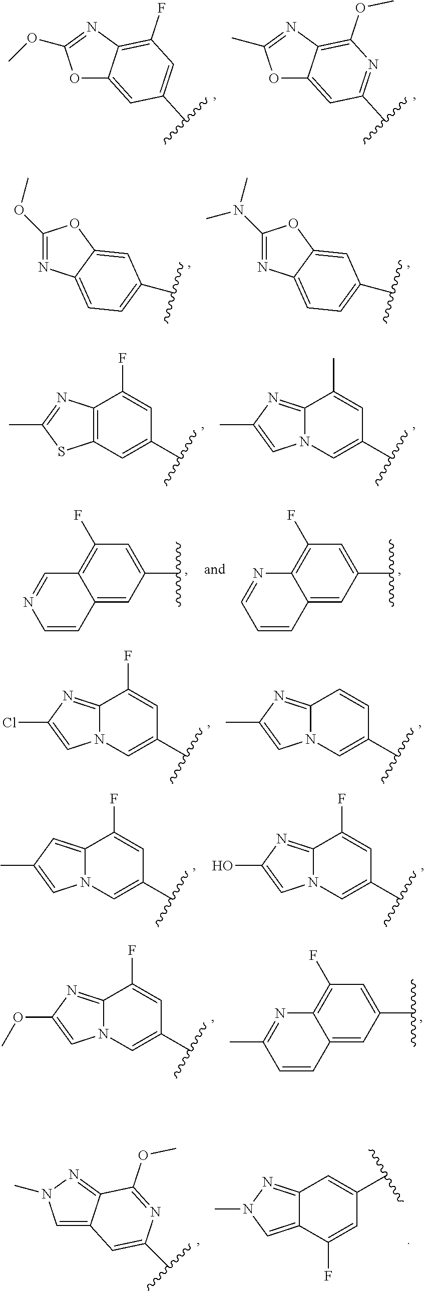

16. The compound of any one of the preceding claims, wherein one of A and B is independently selected from

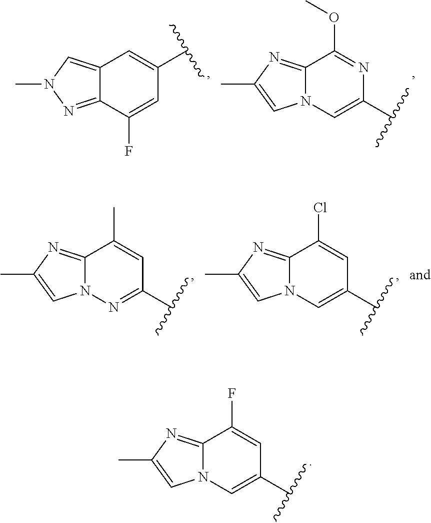 [SMILES: *c1cc(F)c2nn(C)cc2c1 ; *c1cn2cc(C)nc2c(OC)n1 ; *c1cc(C)c2nc(C)cn2n1 ; *c1cc(Cl)c2nc(C)cn2c1 ; *c1cc(F)c2nc(C)cn2c1]

17. The compound of any one of the preceding claims, wherein one of A and B is independently

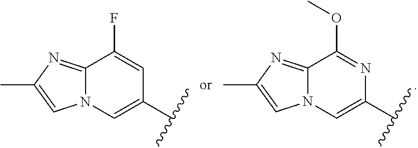 [SMILES: *c1cc(F)c2nc(C)cn2c1 ; *c1cn2cc(C)nc2c(OC)n1]

18. The compound of any one of the preceding claims, wherein A is selected from

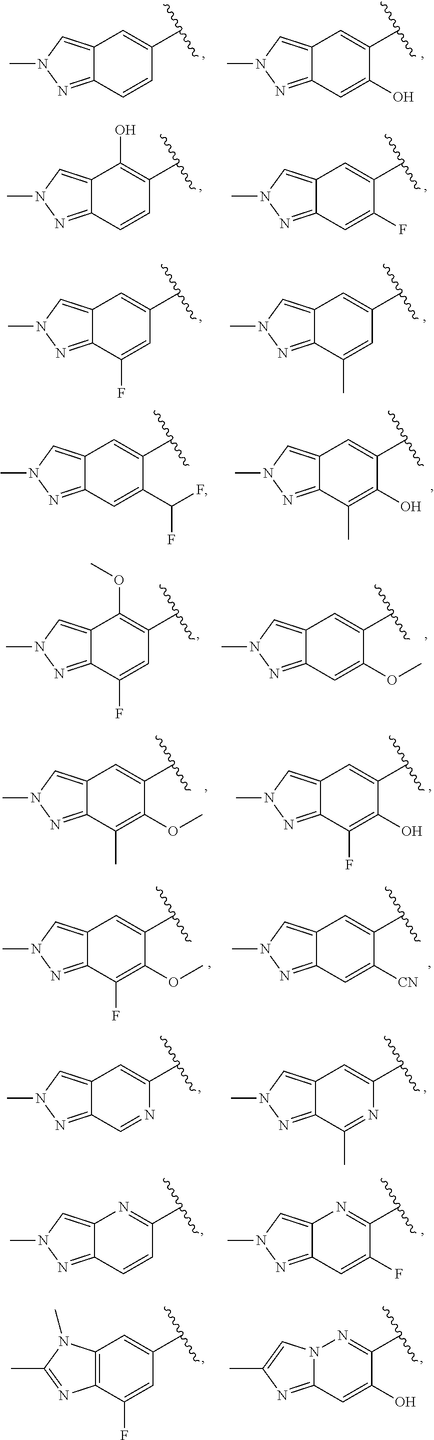

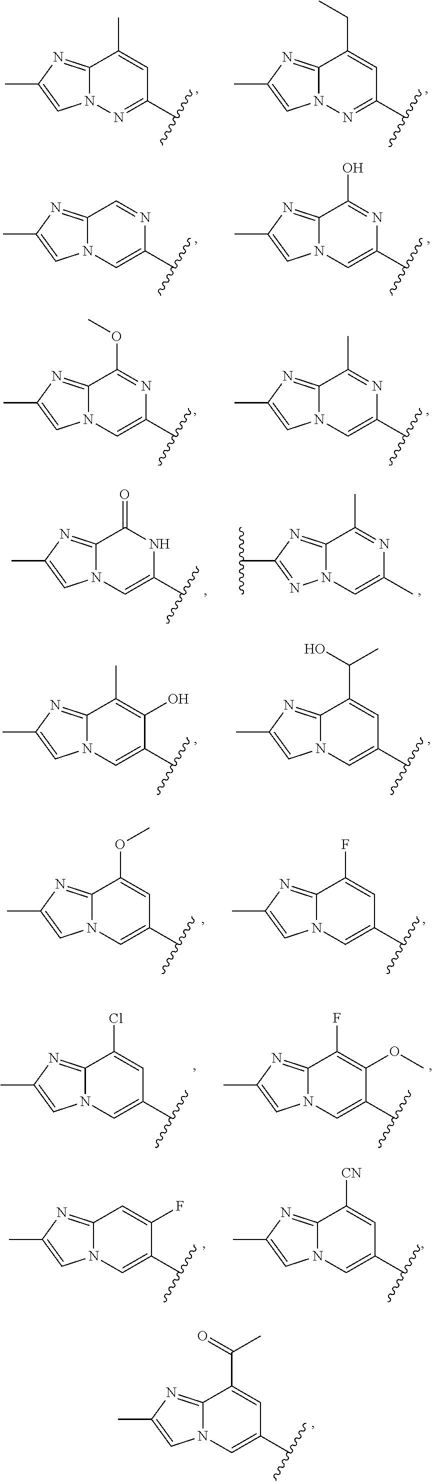

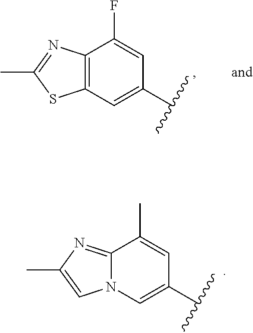 [SMILES: *c1cc(F)c2nc(C)sc2c1 ; *c1cc(C)c2nc(C)cn2c1]

19. The compound of any one of the preceding claims, wherein B is selected from

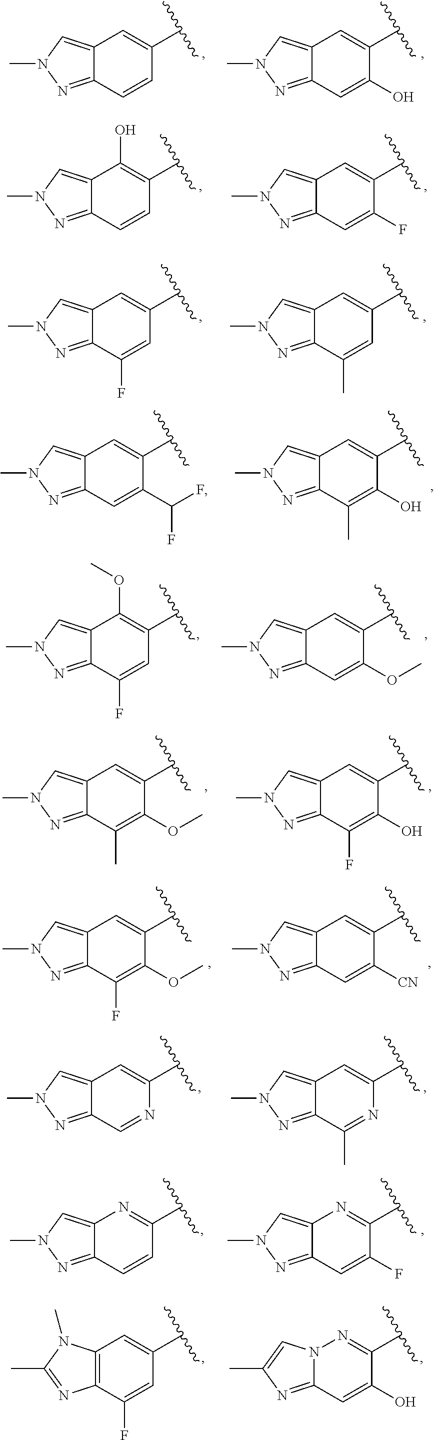

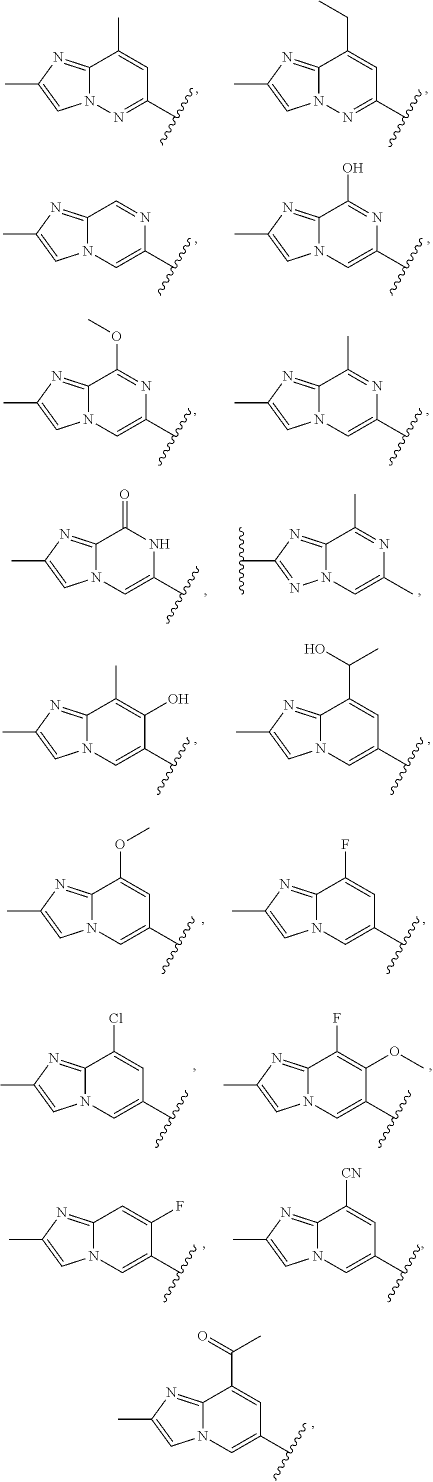

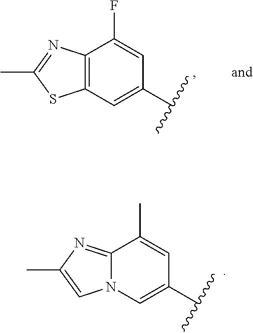 [SMILES: *c1cc(F)c2nc(C)sc2c1 ; *c1cc(C)c2nc(C)cn2c1]

20. The compound of any one of the preceding claims, wherein A selected from

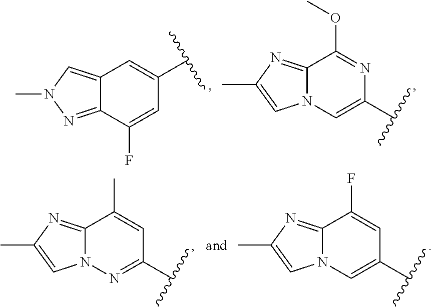

21. The compound of any one of the preceding claims, wherein A is

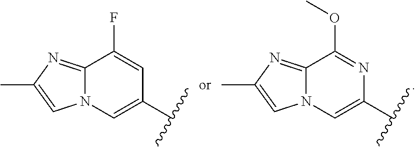 [SMILES: *c1cc(F)c2nc(C)cn2c1 ; *c1cn2cc(C)nc2c(OC)n1]

22. The compound of any one of the preceding claims, wherein one of A and B is independently a monocyclic heterocyclyl or bicyclic heterocyclyl, each of which is optionally substituted with one or more R¹.

23. The compound of any one of the preceding claims, wherein one of A and B is independently a nitrogen-containing heterocyclyl optionally substituted with one or more R¹.

24. The compound of any one of the preceding claims, wherein one of A and B is independently a 4-8 membered heterocyclyl optionally substituted with one or more R¹.

25. The compound of any one of the preceding claims, wherein one of A and B is independently selected from 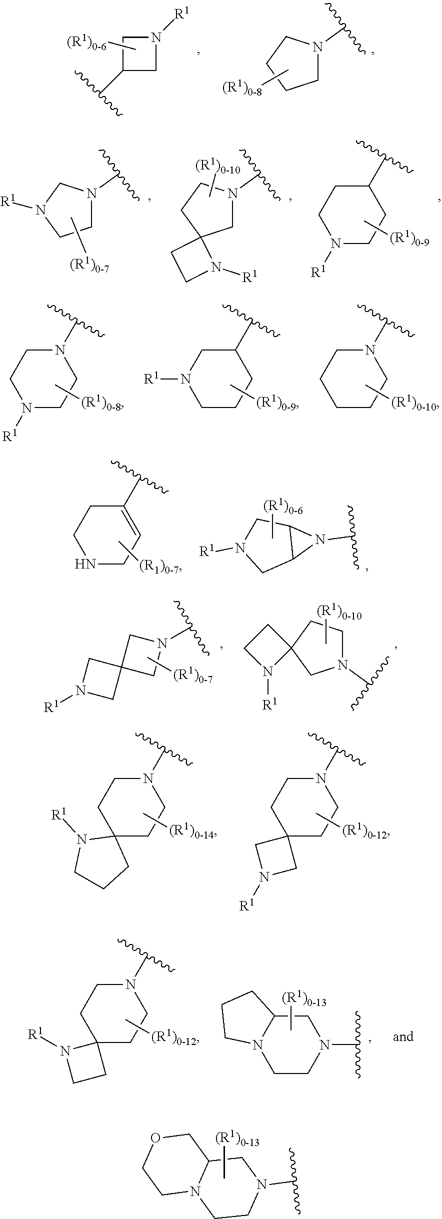
wherein R¹ is as described in claim 1.

26. The compound of any one of the preceding claims, wherein one of A and B is independently selected from 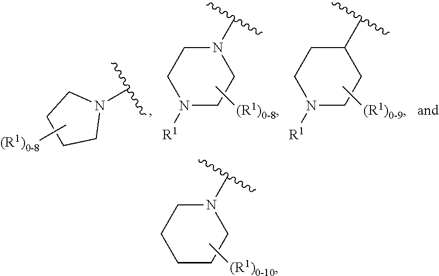
wherein R¹ is as described in claim 1.

27. The compound of any one of the preceding claims, wherein one of A and B is 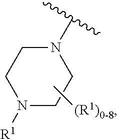
wherein R¹ is as described in claim 1.

28. The compound of any one of the preceding claims, wherein A is selected from 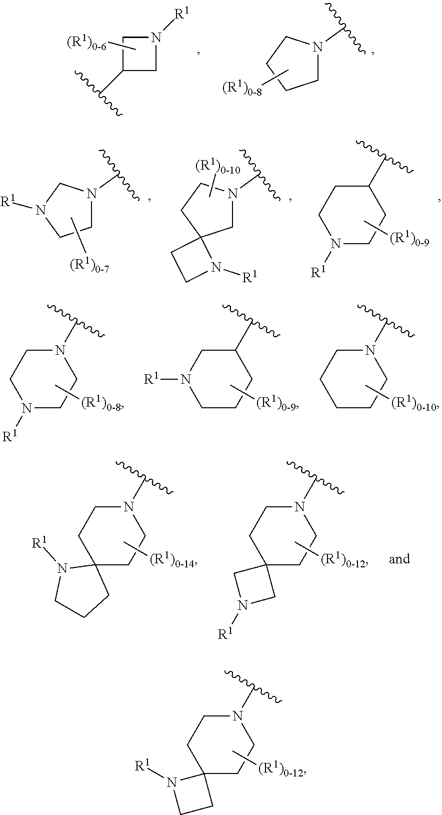
wherein R¹ is as described in claim 1.

29. The compound of any one of the preceding claims, wherein B is selected from 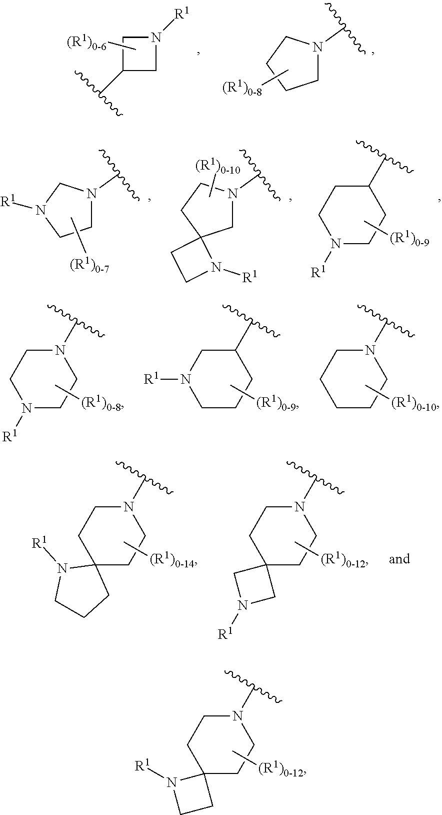
wherein R¹ is as described in claim 1.

30. The compound of any one of the preceding claims, wherein B is selected from 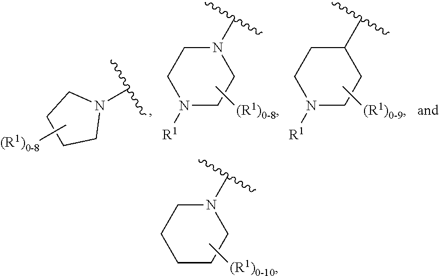
wherein R¹ is as described in claim 1.

31. The compound of any one of the preceding claims, wherein B is 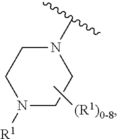
wherein R¹ is as described in claim 1.

32. The compound of any one of the preceding claims, wherein one of A and B is independently selected from

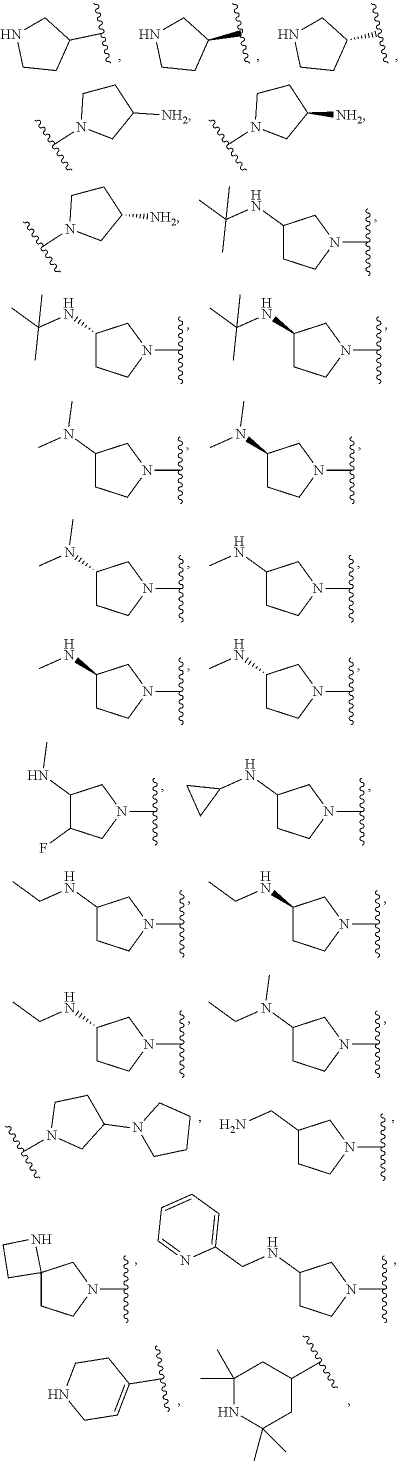

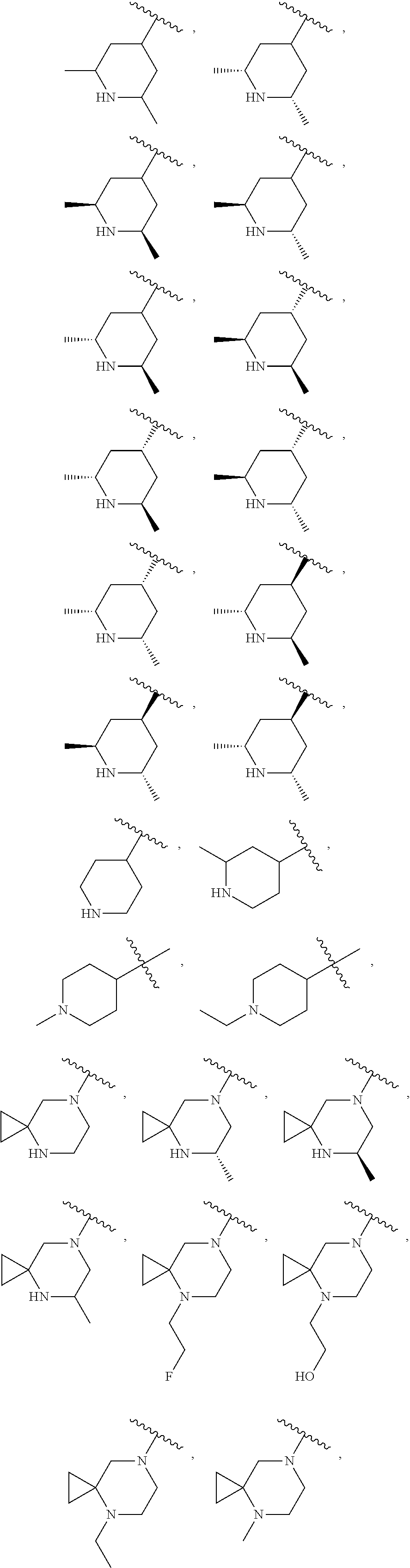

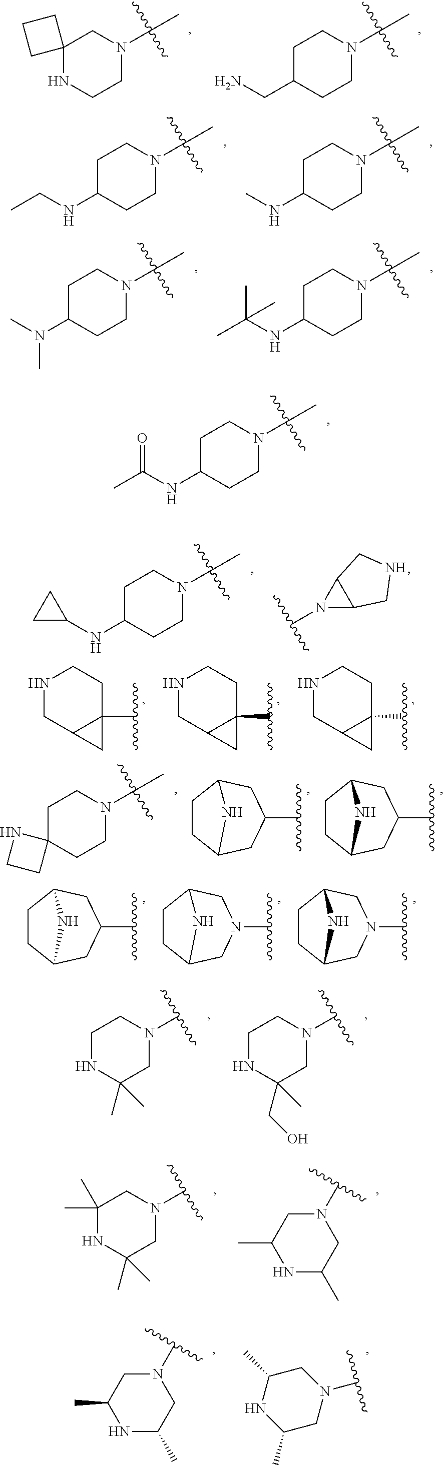

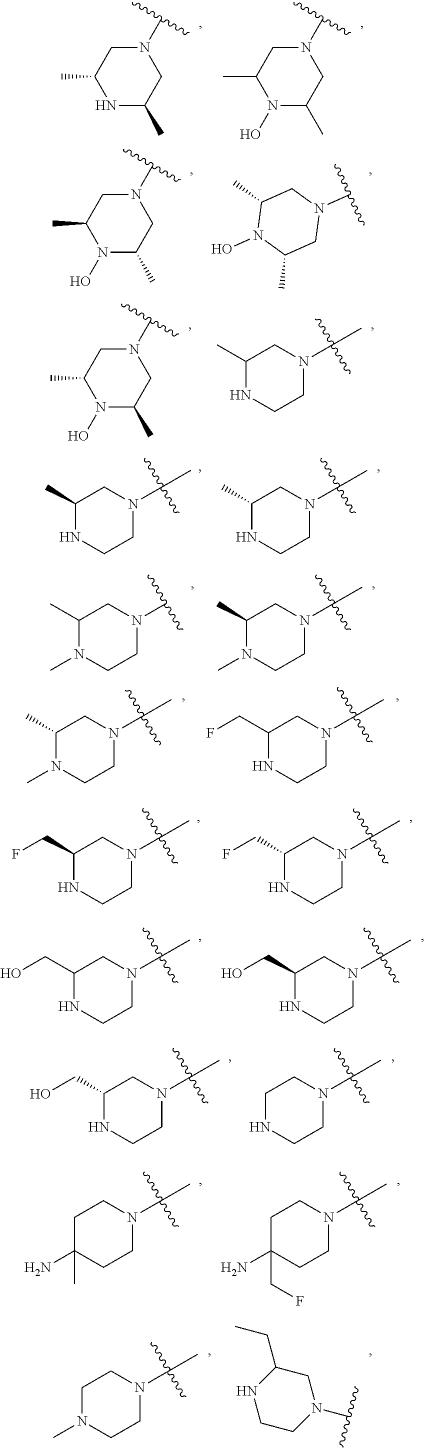

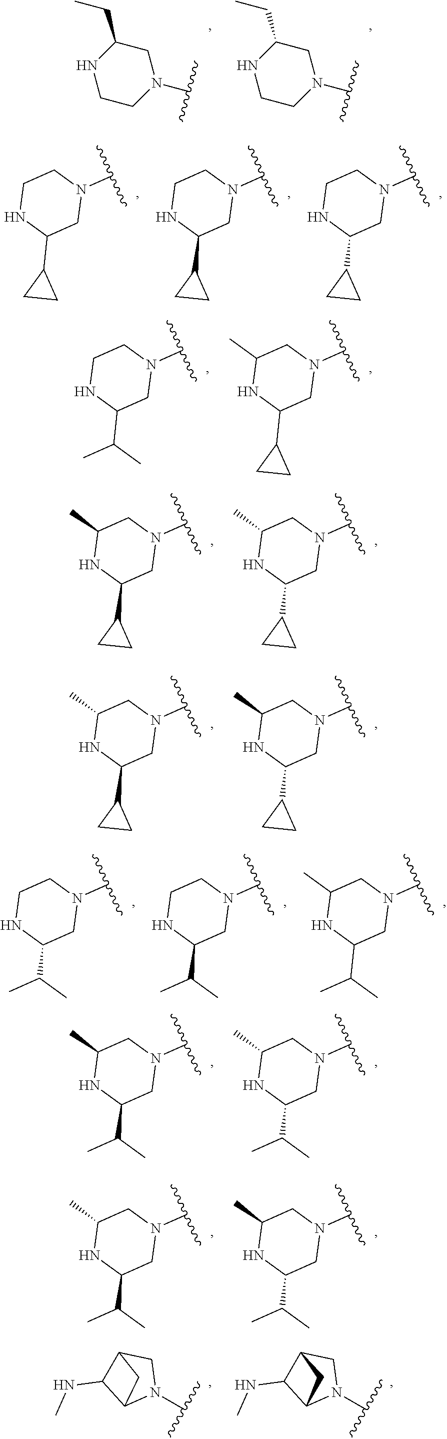

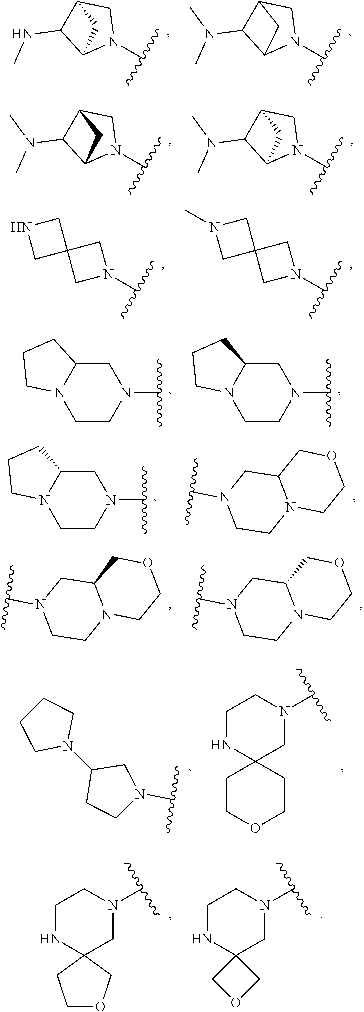

33. The compound of any one of the preceding claims, wherein one of A and B is independently selected from

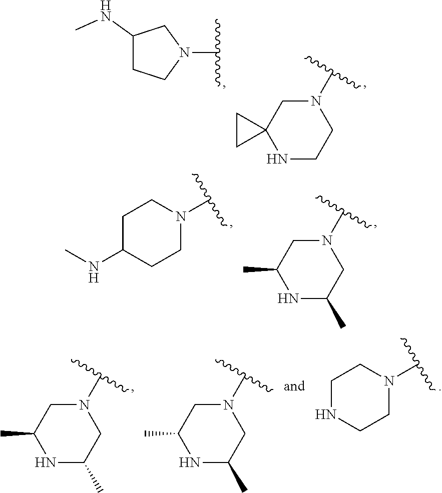

34. The compound of any one of the preceding claims, wherein one of A and B is independently selected from

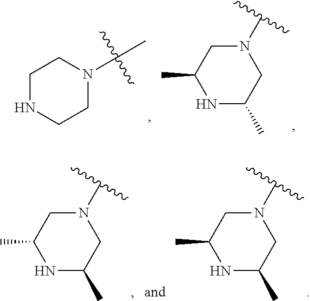

35. The compound of any one of the preceding claims, wherein A is selected from

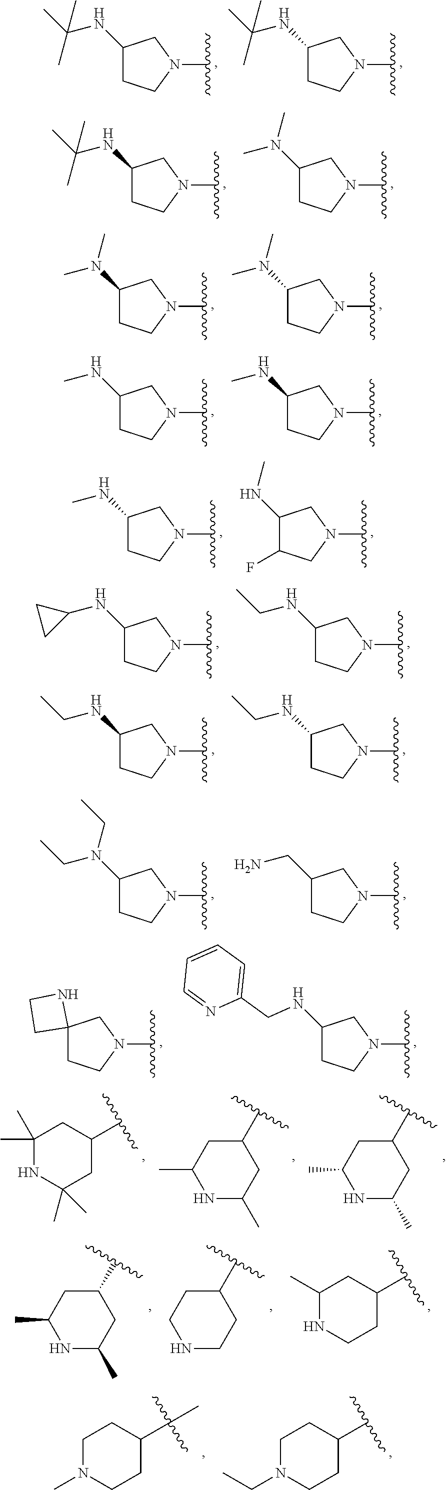

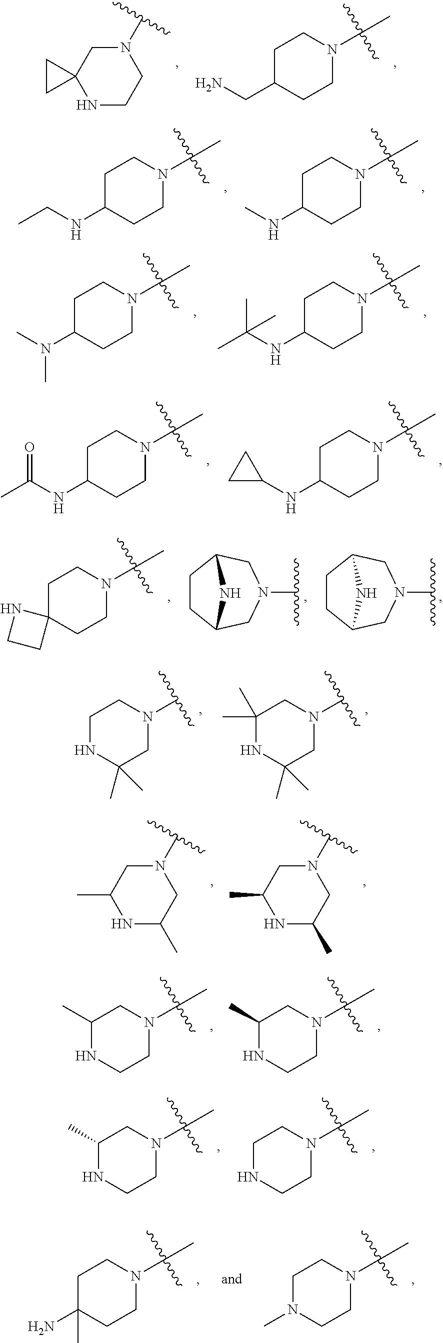

36. The compound of any one of the preceding claims, wherein B is selected from

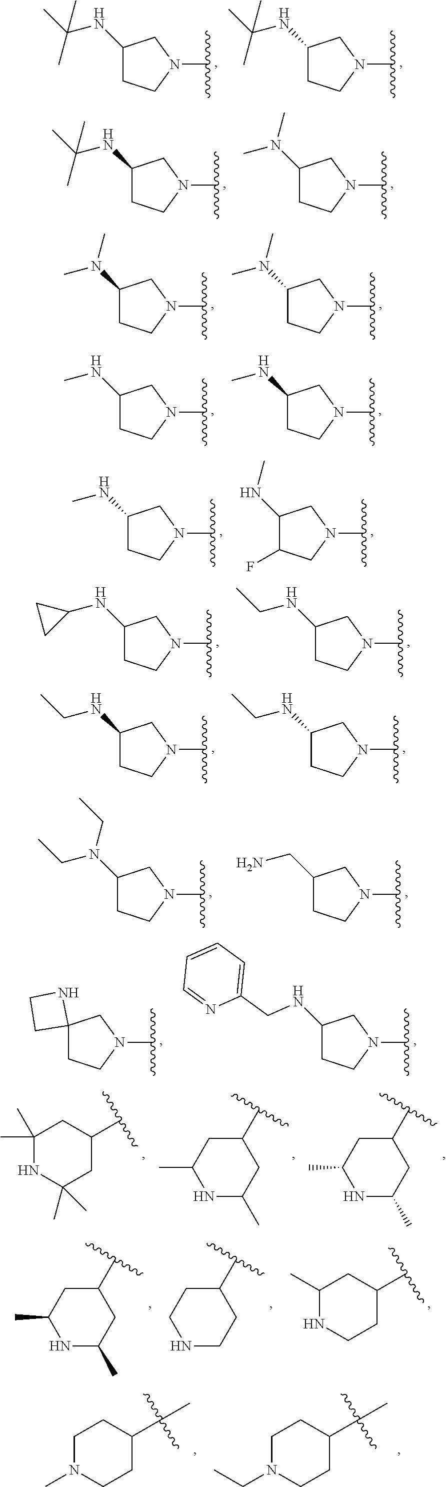

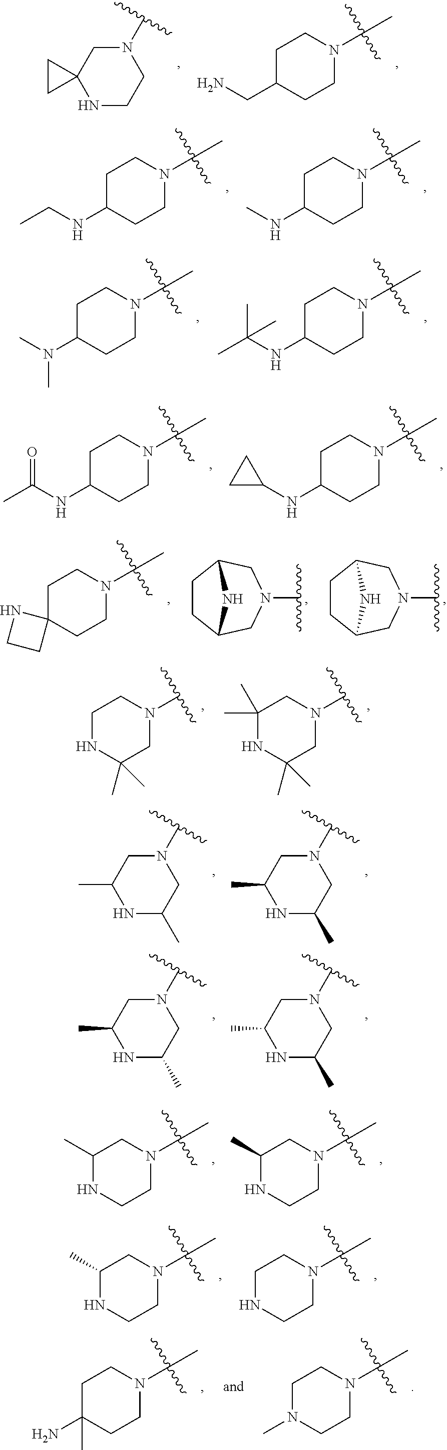

37. The compound of any one of the preceding claims, wherein B is selected from,

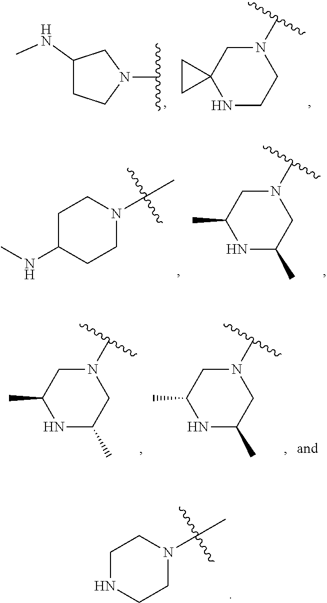

38. The compound of any one of the preceding claims, wherein B is selected from

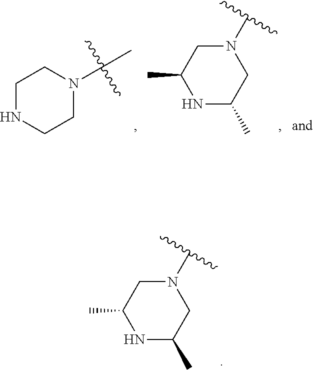

39. The compound of any one of the preceding claims, wherein L¹ is absent, —N(R⁴)C(O)—, or —C(O)N(R⁴)—.

40. The compound of any one of the preceding claims, wherein L¹ is —C(O)N(R⁴)—.

41. The compound of any one of the preceding claims, wherein W is N.

42. The compound of any one of the preceding claims, wherein at least two of W, X, Y, and Z is independently N.

43. The compound of any one of the preceding claims, wherein W and X are each independently N.

44. The compound of any one of the preceding claims, wherein 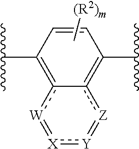
is selected from 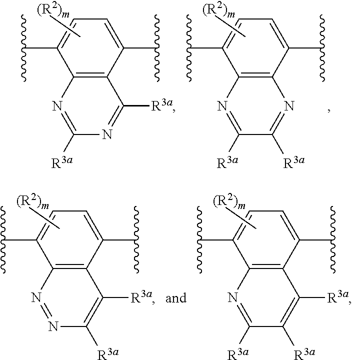
wherein R^{3a} is as defined in claim 1.

45. The compound of any one of the preceding claims, wherein 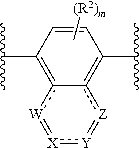
is selected from 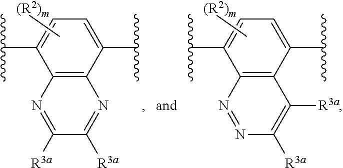
wherein R^{3a} is as defined in claim 1.

46. The compound of any one of the preceding claims, wherein R^{3a} is C₁-C₆-alkyl or C₁-C₆-heteroalkyl.

47. The compound of any one of the preceding claims, wherein the compound of Formula (I) is Formula (I-b): 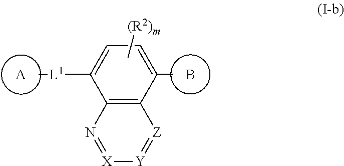
or a pharmaceutically acceptable salt, solvate, hydrate, tautomer, or stereoisomer thereof, wherein each of A, B, L¹, X, Y, Z, R², m, and subvariables thereof is as defined in claim 1.

48. The compound of any one of the preceding claims, wherein the compound of Formula (I) is a compound of Formula (I-c): 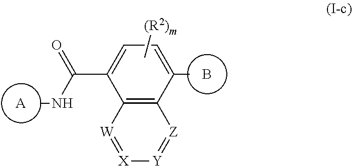
or a pharmaceutically acceptable salt, solvate, hydrate, tautomer, or stereoisomer thereof, wherein each of A, B, W, X, Y, Z, R², m, and subvariables thereof are defined as in claim 1.

49. The compound of any one of the preceding claims, wherein the compound of Formula (I) is a compound of Formula (I-d): 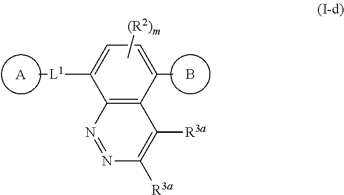
or a pharmaceutically acceptable salt, solvate, hydrate, tautomer, or stereoisomer thereof, wherein each of A, B, L², R², R^{3a}, m, and subvariables thereof are defined as in claim 1.

50. The compound of any one of the preceding claims, wherein the compound of Formula (I) is a compound of Formula (I-e): 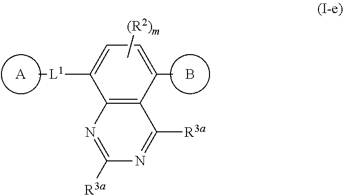
or a pharmaceutically acceptable salt, solvate, hydrate, tautomer, or stereoisomer thereof, wherein each of A, B, L², R², R^{3a}, m, and subvariables thereof are defined as in claim 1.

51. The compound of any one of the preceding claims, wherein the compound of Formula (I) is a compound of Formula (I-f): 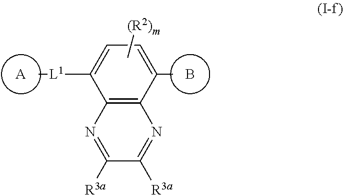
or a pharmaceutically acceptable salt, solvate, hydrate, tautomer, or stereoisomer thereof, wherein each of A, B, L², R², R^{3a}, m, and subvariables thereof are defined as in claim 1.

52. The compound of any one of claims 1-51, wherein:
A is heteroaryl (e.g., bicyclic heteroaryl) optionally substituted with R¹;
B is heterocyclyl (e.g., monocyclic heterocyclyl), optionally substituted with one or more R¹;
L¹ is —C(O)N(R⁴); and
R^{3a} is hydrogen, C₁-C₆-alkyl, or C₁-C₆-heteroalkyl;
wherein each of R¹ and R⁸ are defined as in claim 1.

53. The compound of any one of the preceding claims, wherein the compound of Formula (I) is a compound of Formula (I-g): 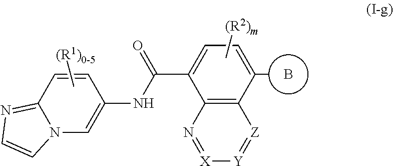
or a pharmaceutically acceptable salt, solvate, hydrate, tautomer, or stereoisomer thereof, wherein each of B, X, Y, Z, R¹, R², m, and subvariables thereof are defined as in claim 1.

54. The compound of any one of the preceding claims, wherein the compound of Formula (I) is a compound of Formula (I-h): 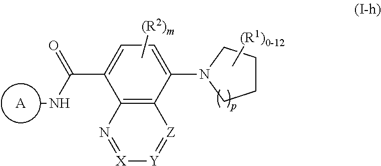
or a pharmaceutically acceptable salt, solvate, hydrate, tautomer, or stereoisomer thereof, wherein p is 0, 1, 2, or 3, and each of A, X, Y, Z, R¹, R², m, p, and subvariables thereof are defined as in claim 1.

55. The compound of any one of the preceding claims, wherein the compound of Formula (I) is a compound of Formula (I-1): 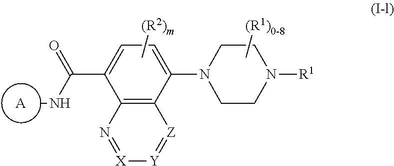
or a pharmaceutically acceptable salt, solvate, hydrate, tautomer, or stereoisomer thereof, wherein p is 0, 1, 2, or 3, and each of A, X, Y, Z, R¹, R², m, and subvariables thereof are defined as in claim 1.

56. The compound of any one of the preceding claims, wherein the compound is selected from a compound listed in Table 1, or a pharmaceutically acceptable salt, solvate, hydrate, tautomer, or stereoisomer thereof.

57. A compound of Formula (II): 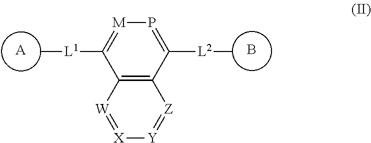
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
M and P are each independently C(R²) or N, wherein at least one of M and P is independently N;
W, X, Y, and Z are each independently C(R³) or N, wherein at least one of W, X, Y, and Z is N;
each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —N^{B}C(O)R^{D}, —NO₂, —C(O)NR^{B}R^{C}, —C(O)OR^{D}, or —S(O)ₓR^{D}, or —C(O)R^{D};
R³ is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —N^{B}C(O)R^{D}, —NO₂, —C(O)NR^{B}R^{C}, —C(O)OR^{D}, or —S(O)ₓR^{D}, or —C(O)R^{D};
each R⁴ is independently hydrogen, C₁-C₆-alkyl, or C₁-C₆-haloalkyl;
each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is C₁-C₆-alkyl, halo, cyano, oxo, or —OR^{A1};
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is hydrogen or C₁-C₆-alkyl; and
x is 0, 1, or 2.

58. The compound of claim 57, wherein each of A and B is independently heteroaryl or heterocyclyl, each of which is optionally substituted with one or more R¹.

59. The compound of any one of the preceding claims, wherein one of A and B is independently a monocyclic heteroaryl or bicyclic heteroaryl, each of which is optionally substituted with one or more R¹.

60. The compound of any one of the preceding claims, wherein one of A and B is independently a bicyclic heteroaryl optionally substituted with one or more R¹.

61. The compound of any one of the preceding claims, wherein one of A and B is independently a nitrogen-containing heteroaryl optionally substituted with one or more R¹.

62. The compound of any one of the preceding claims, wherein one of A and B is a 5-10 membered heteroaryl optionally substituted with one or more R¹.

63. The compound of any one of the preceding claims, wherein one of A and B is independently selected from 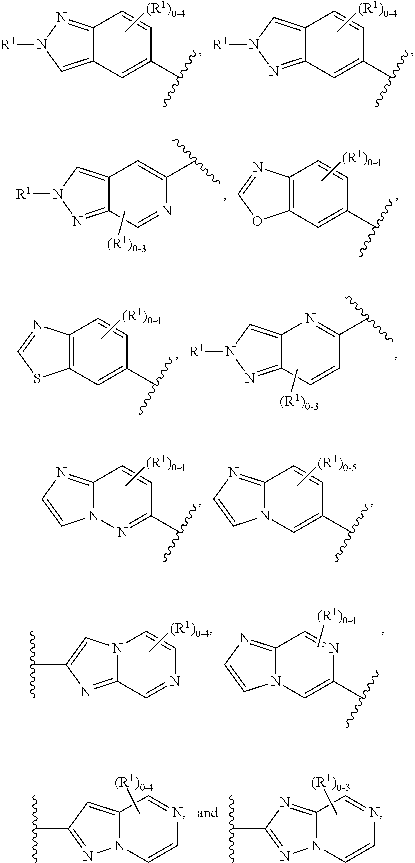
wherein R¹ is as described in claim 57.

64. The compound of any one of the preceding claims, wherein one of A and B is independently selected from 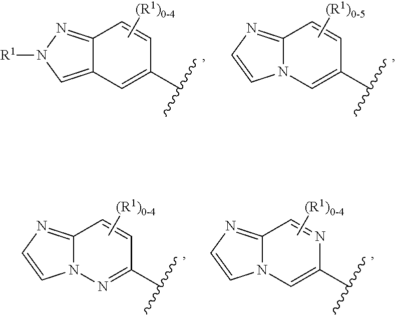
wherein R¹ is as described in claim 57.

65. The compound of any one of the preceding claims, wherein one of A and B is independently selected from 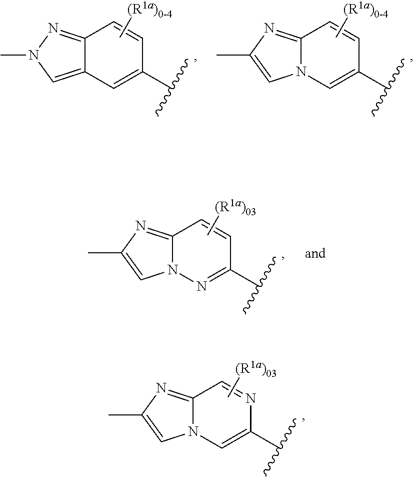
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

66. The compound of any one of the preceding claims, wherein one of A and B is independently 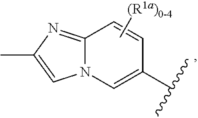
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

67. The compound of any one of the preceding claims, wherein A is selected from 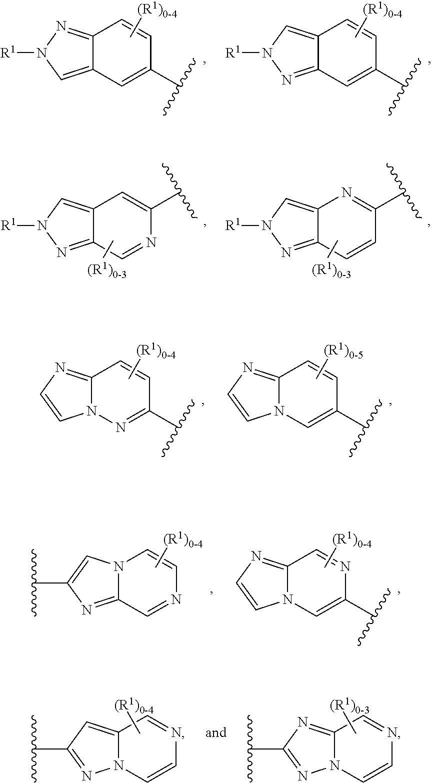
wherein R¹ is as described in claim 57.

68. The compound of any one of the preceding claims, wherein B is selected from 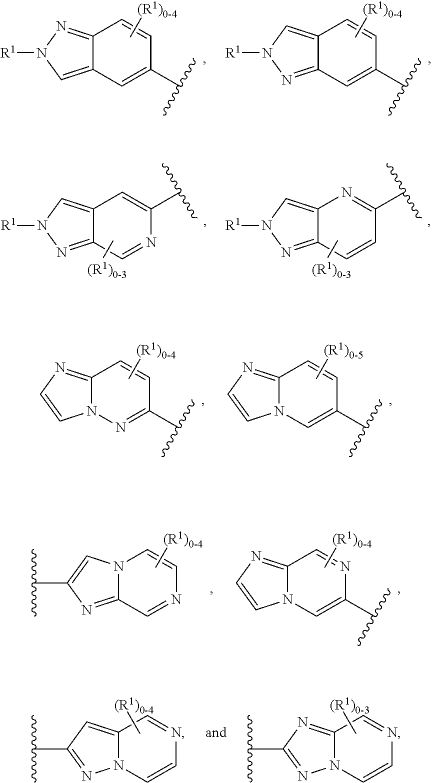
wherein R¹ is as described in claim 57.

69. The compound of any one of the preceding claims, wherein A is selected from 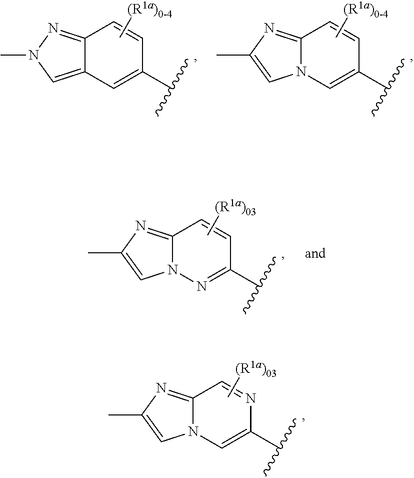
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

70. The compound of any one of the preceding claims, wherein A is 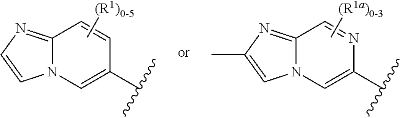
wherein R¹ is as described in claim 57.

71. The compound of any one of the preceding claims, wherein one of A and B is independently selected from

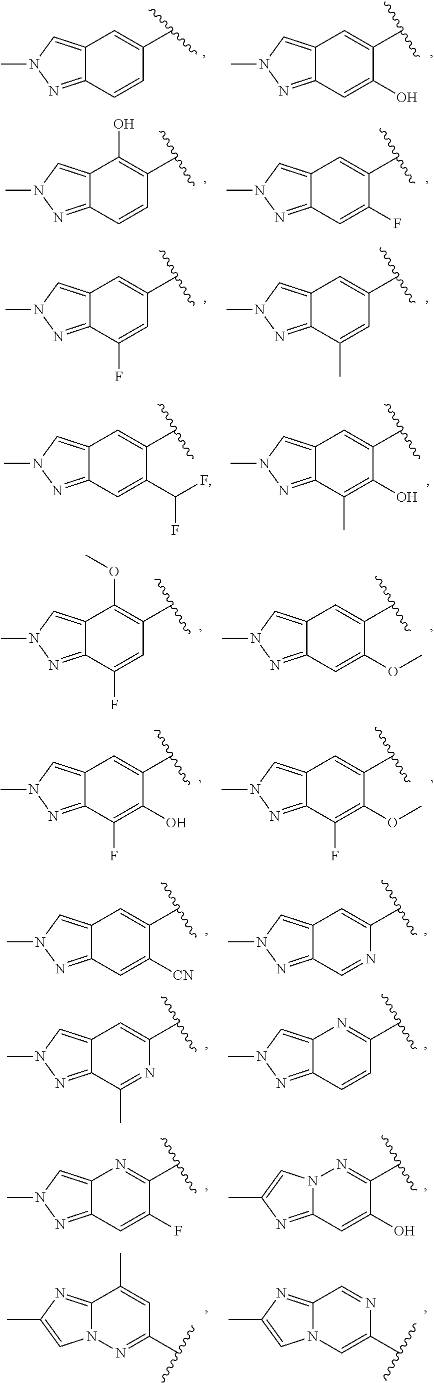

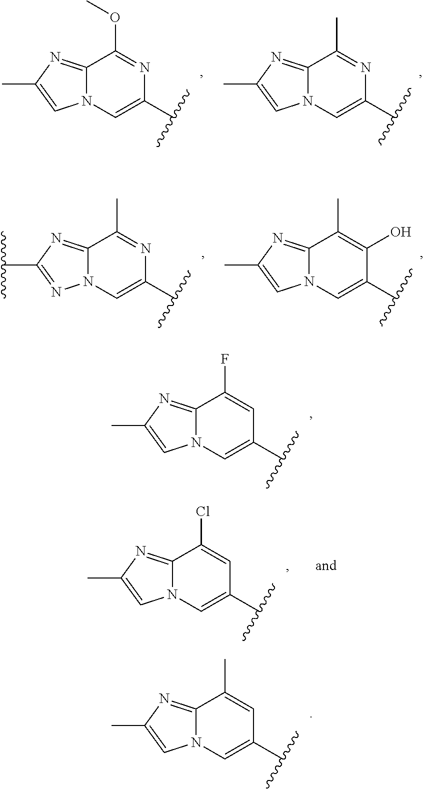

72. The compound of any one of the preceding claims, wherein one of A and B is independently selected from

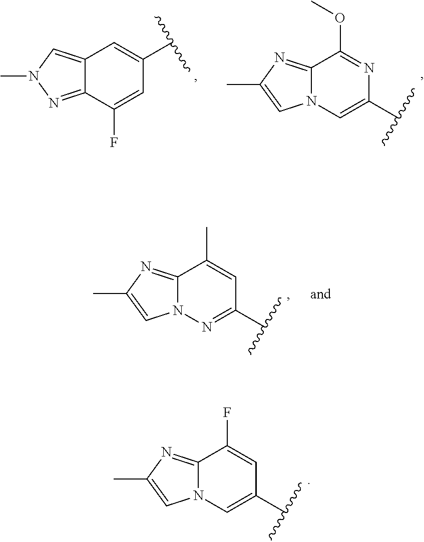 [SMILES: *c1cc(F)c2nn(C)cc2c1 ; *c1cn2cc(C)nc2c(OC)n1 ; *c1cc(C)c2nc(C)cn2n1 ; *c1cc(F)c2nc(C)cn2c1]

73. The compound of any one of the preceding claims, wherein one of A and B is independently

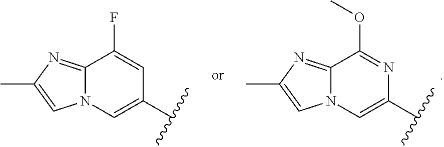 [SMILES: *c1cc(F)c2nc(C)cn2c1 ; *c1cn2cc(C)nc2c(OC)n1]

74. The compound of any one of the preceding claims, wherein A is selected from

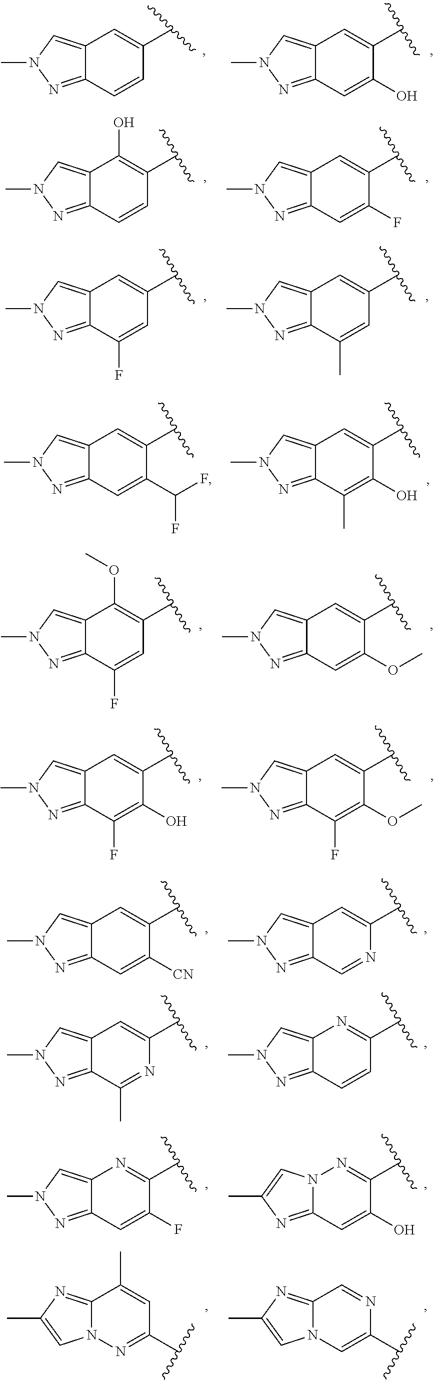

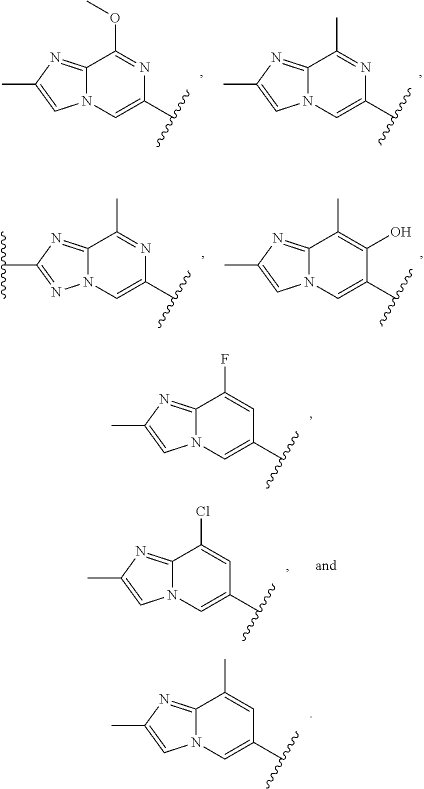

75. The compound of any one of the preceding claims, wherein B is selected from

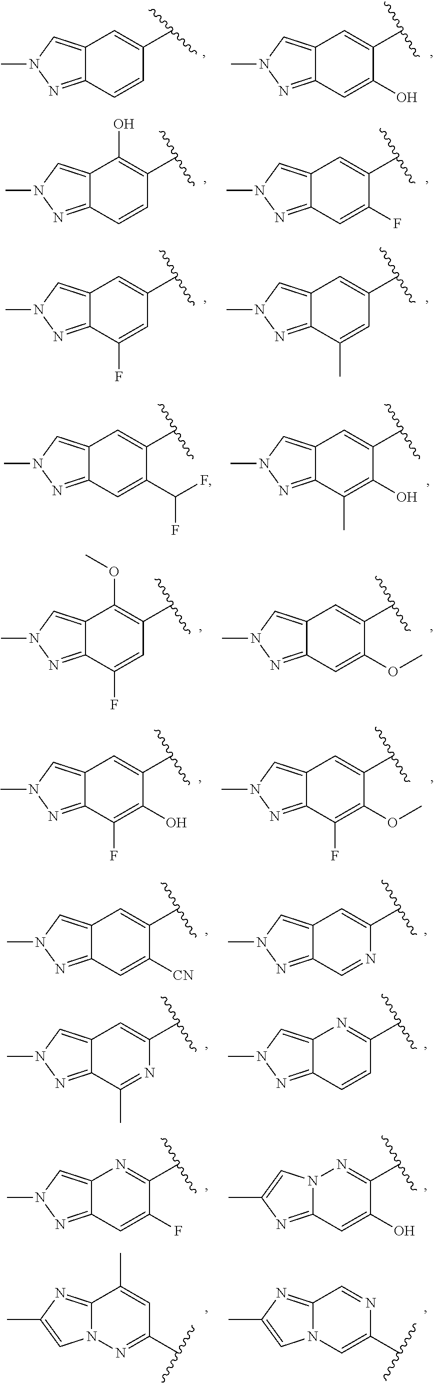

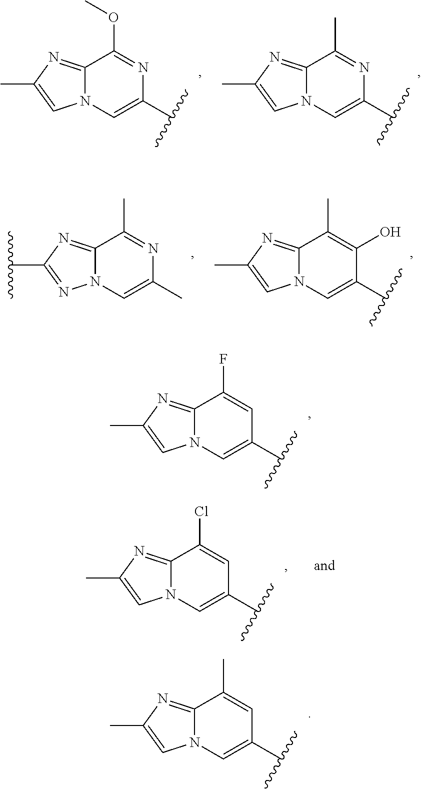

76. The compound of any one of the preceding claims, wherein A selected from

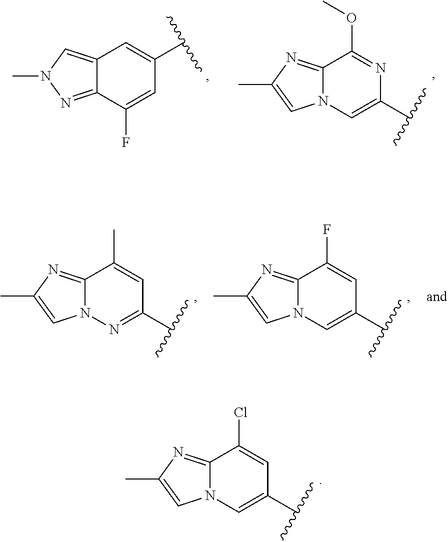 [SMILES: *c1cc(F)c2nn(C)cc2c1 ; *c1cn2cc(C)nc2c(OC)n1 ; *c1cc(C)c2nc(C)cn2n1 ; *c1cc(F)c2nc(C)cn2c1 ; *c1cc(Cl)c2nc(C)cn2c1]

77. The compound of any one of the preceding claims, wherein A is

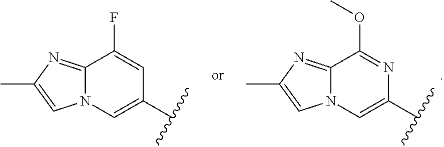 [SMILES: *c1cc(F)c2nc(C)cn2c1 ; *c1cn2cc(C)nc2c(OC)n1]

78. The compound of any one of the preceding claims, wherein one of A and B is independently a monocyclic heterocyclyl or bicyclic heterocyclyl, each of which is optionally substituted with one or more R¹.

79. The compound of any one of the preceding claims, wherein one of A and B is independently a nitrogen-containing heterocyclyl optionally substituted with one or more R¹.

80. The compound of any one of the preceding claims, wherein one of A and B is independently a 4-8 membered heterocyclyl optionally substituted with one or more R¹.

81. The compound of any one of the preceding claims, wherein one of A and B is independently selected from 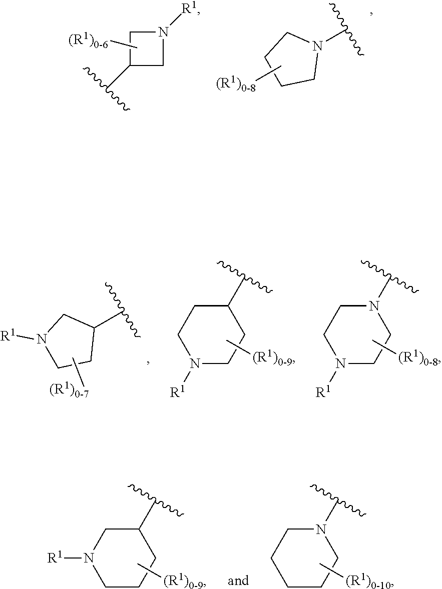
wherein R¹ is as described in claim 1.

82. The compound of any one of the preceding claims, wherein one of A and B is independently selected from 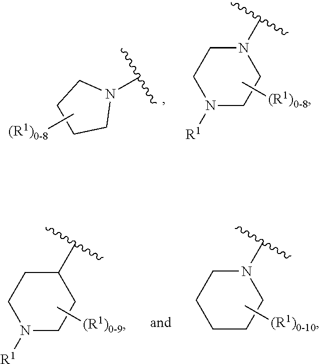
wherein R¹ is as described in claim 57.

83. The compound of any one of the preceding claims, wherein one of A and B is selected from 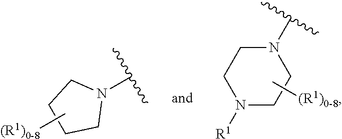
wherein R¹ is as described in claim 57.

84. The compound of any one of the preceding claims, wherein A is selected from 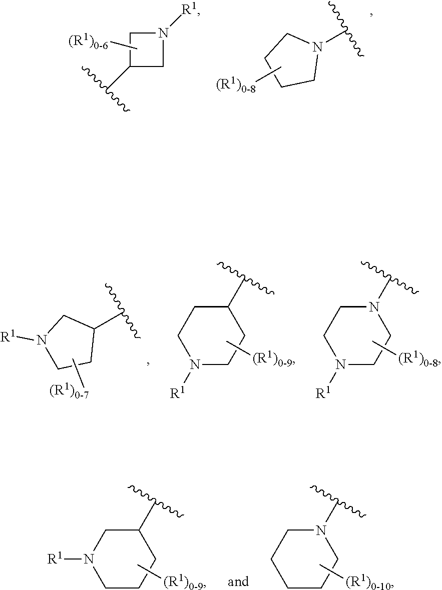
wherein R¹ is as described in claim 57.

85. The compound of any one of the preceding claims, wherein B is selected from 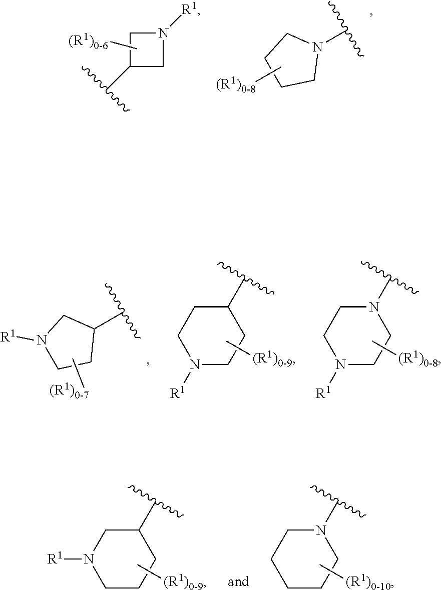
wherein R¹ is as described in claim 57.

86. The compound of any one of the preceding claims, wherein B is selected from 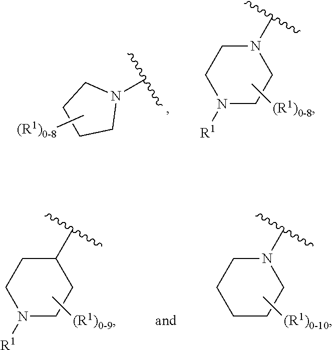
wherein R¹ is as described in claim 57.

87. The compound of any one of the preceding claims, wherein B is 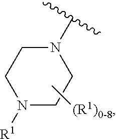
wherein R¹ is as described in claim 57.

88. The compound of any one of the preceding claims, wherein one of A and B is independently selected from

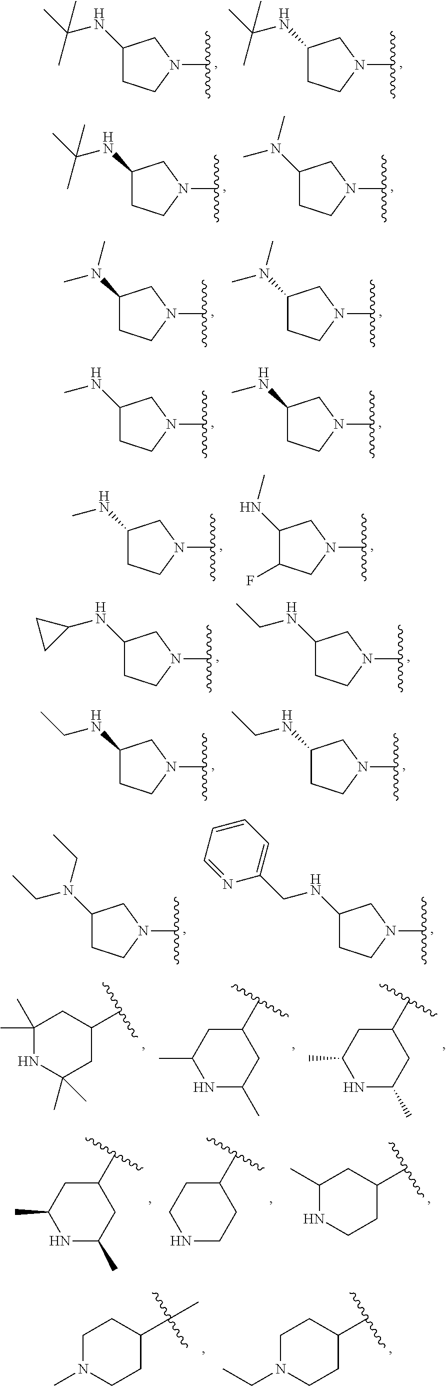

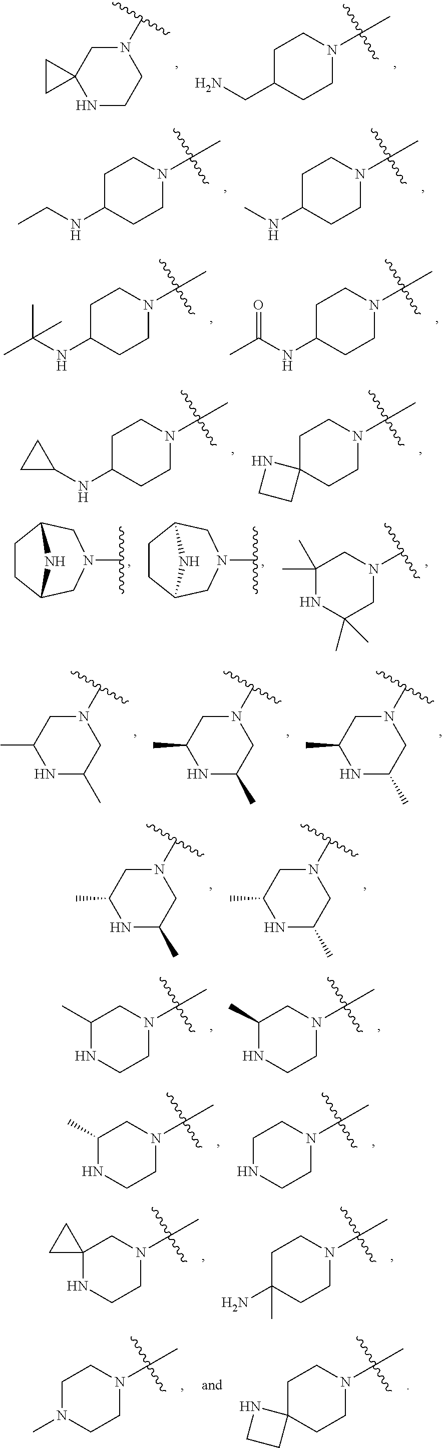

89. The compound of any one of the preceding claims, wherein one of A and B is independently selected from,

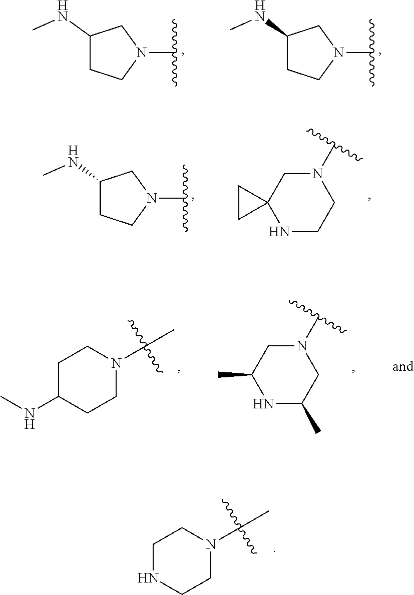

90. The compound of any one of the preceding claims, wherein one of A and B is selected from

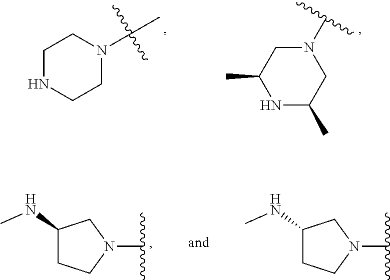

91. The compound of any one of the preceding claims, wherein A is selected from

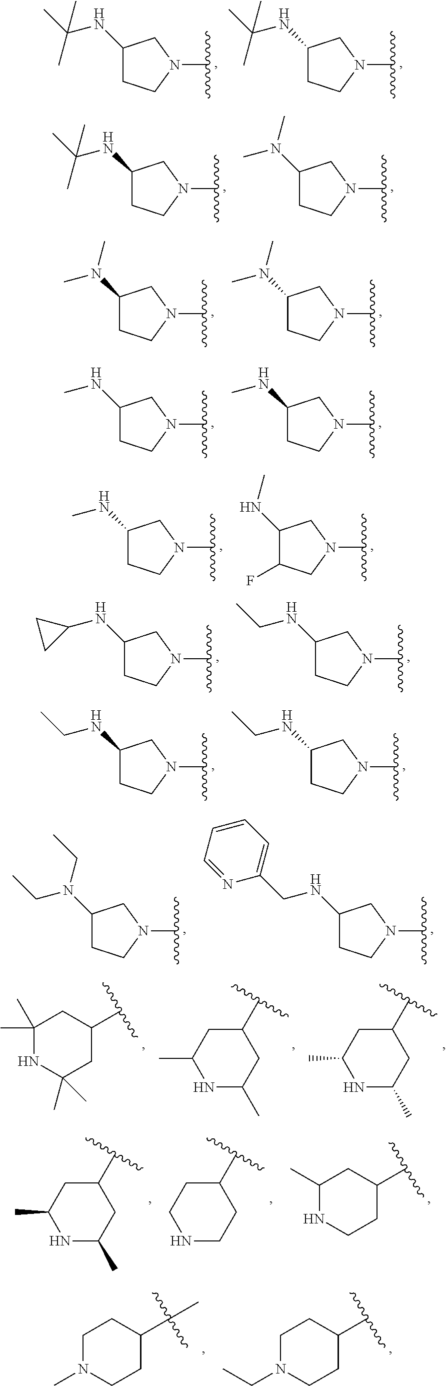

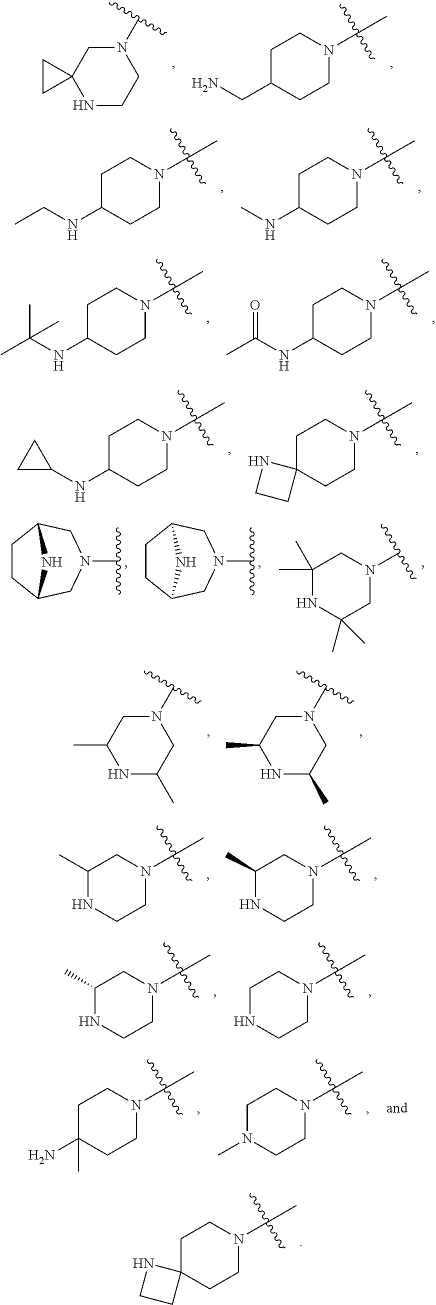

92. The compound of any one of the preceding claims, wherein B is selected from

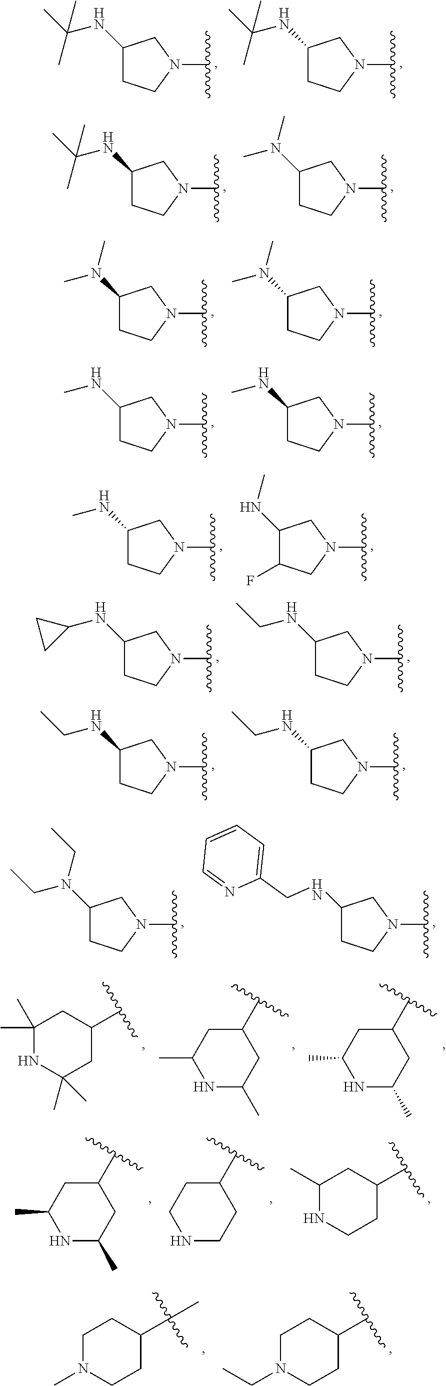

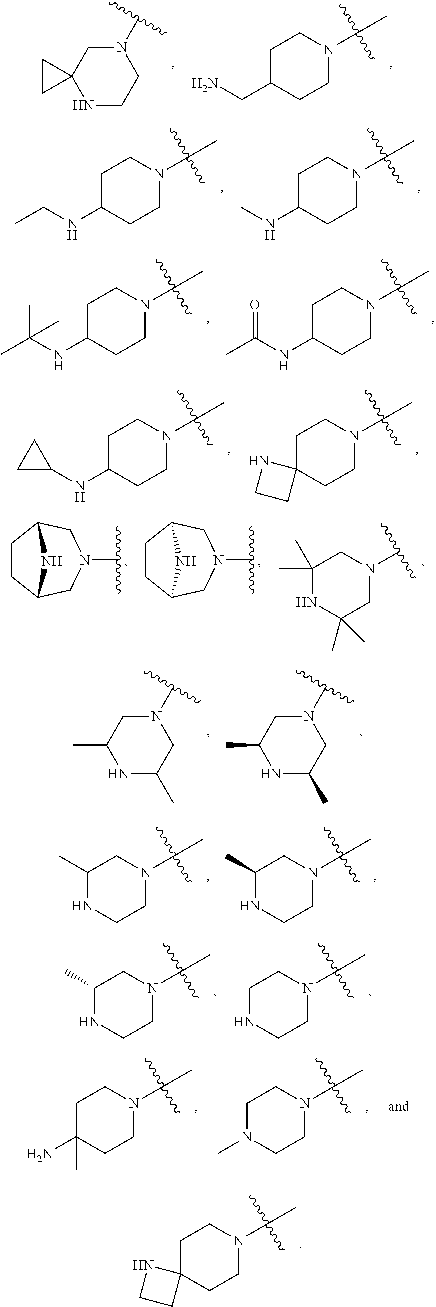

93. The compound of any one of the preceding claims, wherein B is selected from,

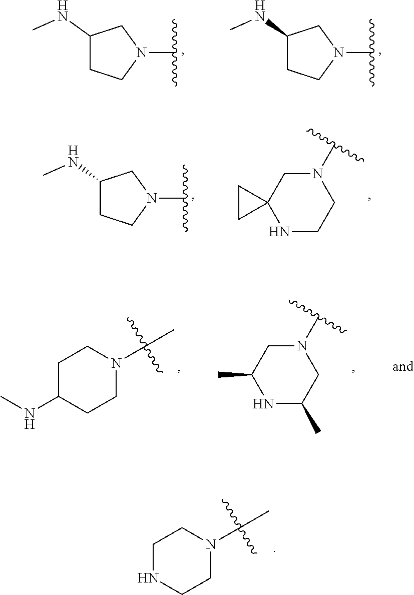

94. The compound of any one of the preceding claims, wherein B is selected from

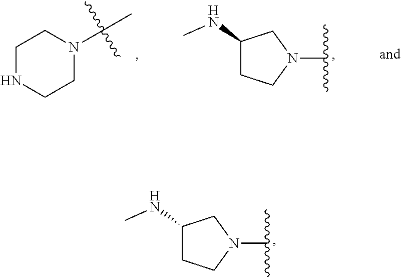

95. The compound of any one of the preceding claims, wherein one of L^{C} and L² is independently absent, —N(R⁴)C(O)—, or —C(O)N(R⁴)—.

96. The compound of any one of the preceding claims, wherein each of L¹ and L² is independently absent, —N(R⁴)C(O)—, or —C(O)N(R⁴)—.

97. The compound of any one of the preceding claims, wherein each of L¹ and L² is independently absent.

98. The compound of any one of the preceding claims, wherein L¹ is —C(O)N(R⁴)—.

99. The compound of any one of the preceding claims, wherein L² is absent.

100. The compound of any one of the preceding claims, wherein L¹ is —C(O)NH— and L² is absent.

101. The compound of any one of the preceding claims, wherein each of M and P is independently C(R²) (e.g., CH).

102. The compound of any one of the preceding claims, wherein M is C(R²) (e.g., CH) and P is N.

103. The compound of any one of the preceding claims, wherein M is N and P is C(R²) (e.g., CH).

104. The compound of any one of the preceding claims, wherein W is independently N.

105. The compound of any one of the preceding claims, wherein X is N.

106. The compound of any one of the preceding claims, wherein Y is N.

107. The compound of any one of the preceding claims, wherein Z is N.

108. The compound of any one of the preceding claims, wherein one of W and X is independently N.

109. The compound of any one of the preceding claims, wherein two of W, X, Y, and Z are independently N.

110. The compound of any one of the preceding claims, wherein 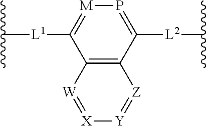
is selected from N and N

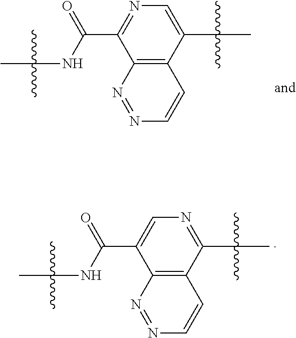 [SMILES: *NC(=O)c1ncc(*)c2ccnnc12 ; *NC(=O)c1cnc(*)c2ccnnc12]

111. The compound of any one of the preceding claims, wherein the compound of Formula (II) is Formula (II-a): 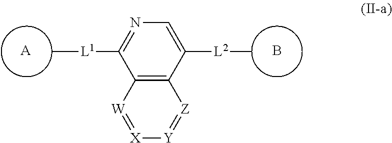
or a pharmaceutically acceptable salt, solvate, hydrate, tautomer, or stereoisomer thereof, wherein each of A, B, W, X, Y, Z, M, P, L¹, L², and subvariables thereof are defined as in claim 57.

112. The compound of any one of the preceding claims, wherein the compound of Formula (II) is Formula (II-b): 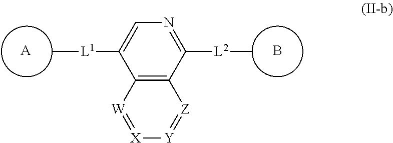
or a pharmaceutically acceptable salt, solvate, hydrate, tautomer, or stereoisomer thereof, wherein each of A, B, W, X, Y, Z, L¹, L², and subvariables thereof are defined as in claim 57.

113. The compound of any one of the preceding claims, wherein the compound of Formula (II) is Formula (II-c): 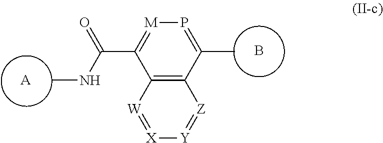
or a pharmaceutically acceptable salt, solvate, hydrate, tautomer, or stereoisomer thereof, wherein each of A, B, W, X, Y, Z, M, P, and subvariables thereof are defined as in claim 57.

114. The compound of any one of the preceding claims, wherein the compound of Formula (II) is Formula (II-d): 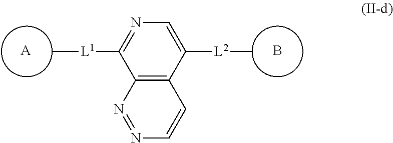
or a pharmaceutically acceptable salt, solvate, hydrate, tautomer, or stereoisomer thereof, wherein each of A, B, L¹, L², and subvariables thereof are defined as in claim 57.

115. The compound of any one of the preceding claims, wherein the compound of Formula (II) is Formula (II-e): 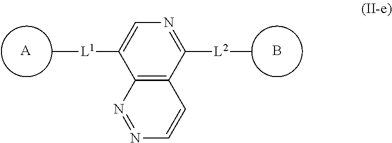
or a pharmaceutically acceptable salt, solvate, hydrate, tautomer, or stereoisomer thereof, wherein each of A, B, L¹, L², and subvariables thereof are defined as in claim 1.

116. The compound of any one of the preceding claims, wherein the compound is selected from any one of the compounds shown in Table 2 or a pharmaceutically acceptable salt, solvate, hydrate, tautomer, or stereoisomer thereof.

117. A pharmaceutical composition comprising a compound of any one of the preceding claims and a pharmaceutically acceptable excipient.

118. The compound of any one of claims 1-118, or the pharmaceutical composition of claim 117, wherein the compound alters a target nucleic acid (e.g., an RNA, e.g., a pre-mRNA).

119. The compound of any one of claims 1-118, or the pharmaceutical composition of claim 117, wherein the compound binds to a target nucleic acid (e.g., an RNA, e.g., a pre-mRNA).

120. The compound of any one of claims 1-118, or the pharmaceutical composition of claim 117, wherein the compound stabilizes a target nucleic acid (e.g., an RNA, e.g., a pre-mRNA).

121. The compound of any one of claims 1-118, or the pharmaceutical composition of claim 117, wherein the compound increases splicing at splice site on a target nucleic acid (e.g., an RNA, e.g., a pre-mRNA), by about 0.5%, 1%, 2%, 3%, 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

122. The compound of any one of claims 1-118, or the pharmaceutical composition of claim 117, wherein the compound decreases splicing at splice site on a target nucleic acid (e.g., an RNA, e.g., a pre-mRNA), by about 0.5%, 1%, 2%, 3% 4%, 5%, 6%, 7% 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

123. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), or (III), described herein:
comprising contacting the nucleic acid (e.g., a DNA, RNA, e.g., a pre-mRNA) with a compound of Formula (I), (II), or (III).

124. The method of claim 123, wherein the component of a spliceosome is recruited to the nucleic acid in the presence of the compound of Formula (I), (II), or (III) or a pharmaceutically acceptable salt or a composition thereof.

125. A method of altering the conformation of a nucleic acid (e.g., a DNA, RNA, e.g., a pre-mRNA) comprising contacting the nucleic acid with a compound of Formula (I), (II), or (III) or a pharmaceutically acceptable salt or a composition thereof.

126. The method of claim 125, wherein the altering comprises forming a bulge in the nucleic acid.

127. The method of claim 125, wherein the altering comprises stabilizing a bulge in the nucleic acid.

128. The method of claim 125, wherein the altering comprises reducing a bulge in the nucleic acid.

129. The method of any one of any one of claims 64-67, wherein the nucleic acid comprises a splice site.

130. A method for treating a disease or disorder in a subject comprising administering to the subject a a compound of Formula (I), (II), or (III) or a pharmaceutically acceptable salt or a composition thereof.

131. The method of claim 130, wherein the disease or disorder comprises a proliferative disease (e.g., cancer, a benign neoplasm, or angiogenesis).

132. The method of any one of claims 130-131, wherein the proliferative disease is cancer.

133. The method of any one of claims 130-132, wherein the proliferative disease comprises adenoid cystic carcinoma, colorectal cancer, leukemia, lung cancer, prostate cancer, breast cancer, or ovarian cancer

134. The method of claim 130, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

135. The method of claim 134, wherein the disease or disorder comprises neurological disease or disorder.

136. The method of claim 134, wherein the disease or disorder comprises Huntington's disease.

137. A method of treating a proliferative disease in a subject comprising administering to the subject a compound of Formula (III) or a composition comprising a compound of Formula (III) and a pharmaceutically acceptable excipient, wherein the compound of Formula (III) is: 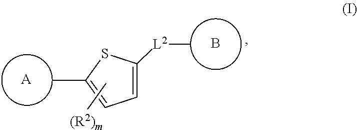
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L² is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A};
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R⁷ is independently C₁-C₆-alkyl or halo;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁷; or
R^{B} and R^{C} together with the nitrogen atom to which they are attached form a 3-7-membered heterocyclyl or heteroaryl, wherein each heterocyclyl or heteroaryl is optionally substituted with one or more R⁷;
each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
m is 0, 1, or 2; and
x is 0, 1, or 2.

138. The method of claim 137, wherein one of A and B is independently a monocyclic heterocyclyl or bicyclic heterocyclyl, each of which is optionally substituted with one or more R¹.

139. The method of any one of the preceding claims, wherein one of A and B is independently a nitrogen-containing heterocyclyl optionally substituted with one or more R¹.

140. The method of any one of the preceding claims, wherein one of A and B is independently a 4-8 membered heterocyclyl optionally substituted with one or more R¹.

141. The method of any one of the preceding claims, wherein one of A and B is independently selected from 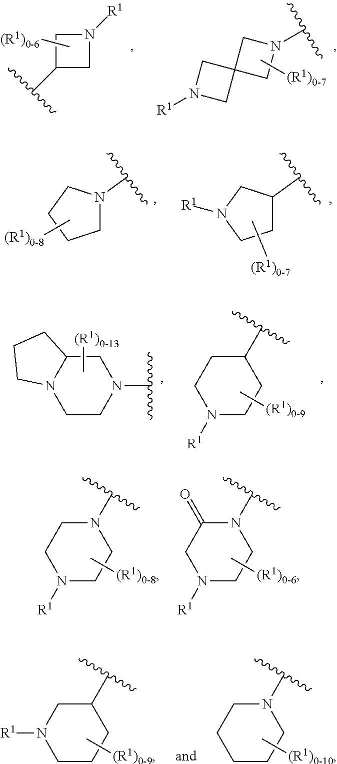
wherein R¹ is as described in claim 137.

142. The method of any one of the preceding claims, wherein one of A and B is 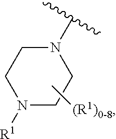
wherein R¹ is as described in claim 137.

143. The method of any one of the preceding claims, wherein A is selected from 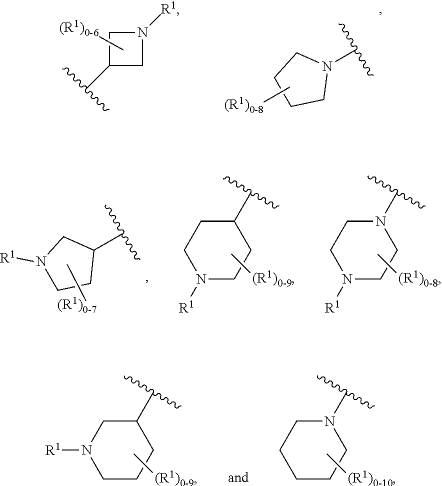
and R¹ is as described in claim 137.

144. The method of any one of the preceding claims, wherein B is selected from 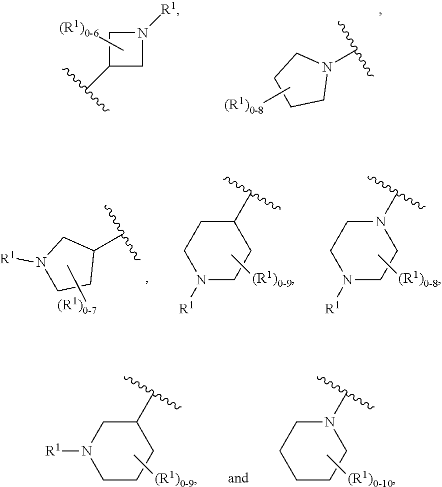
and R¹ is as described in claim 137.

145. The method of any one of the preceding claims, wherein A is 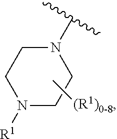
and R¹ is as described in claim 137.

146. The method of any one of the preceding claims, wherein one of A and B is independently selected from

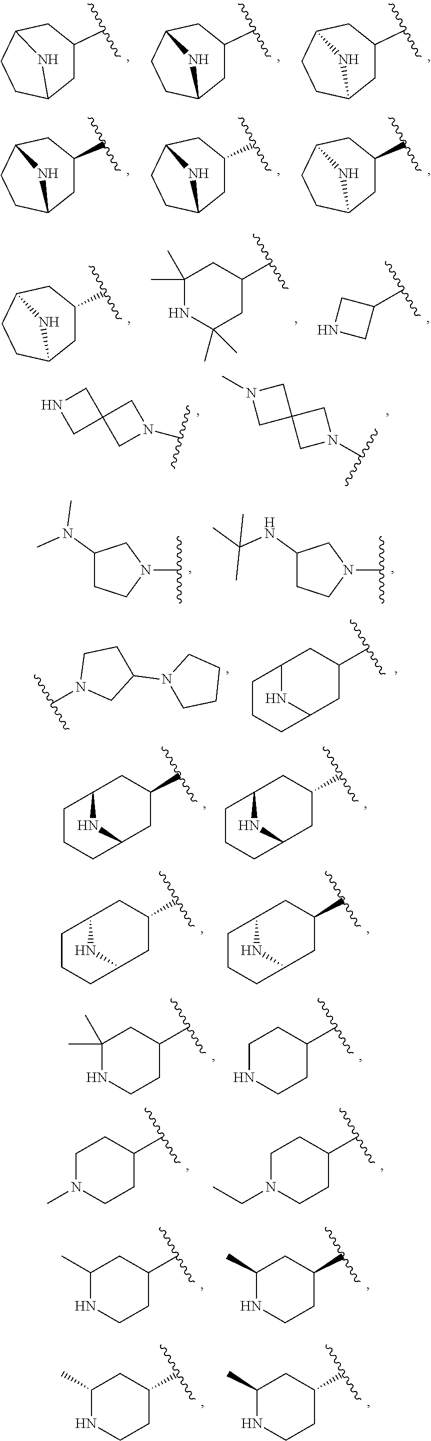

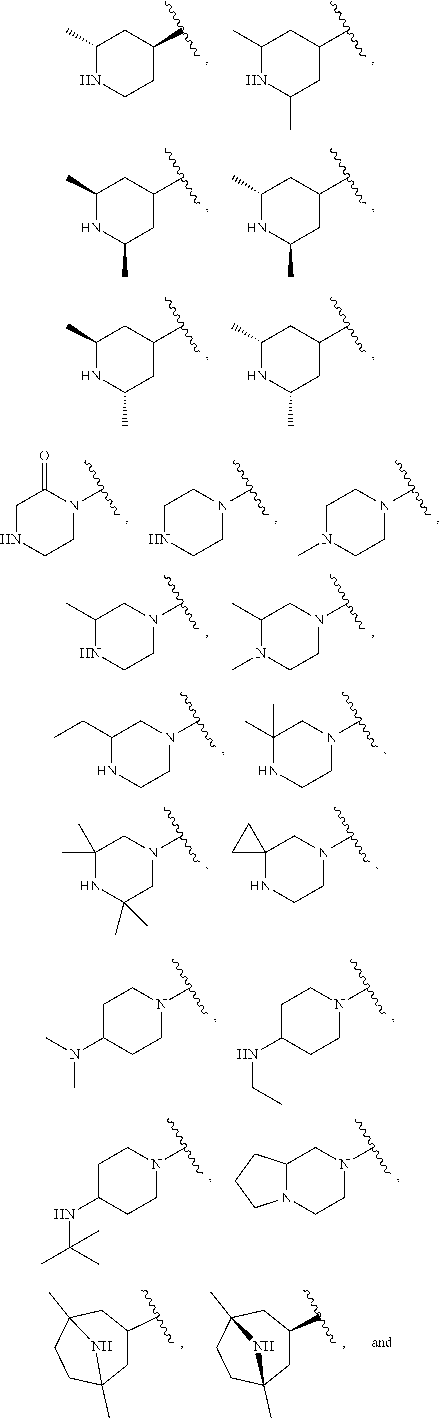

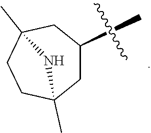

147. The method of any one of the preceding claims, wherein one of A and B is independently selected from

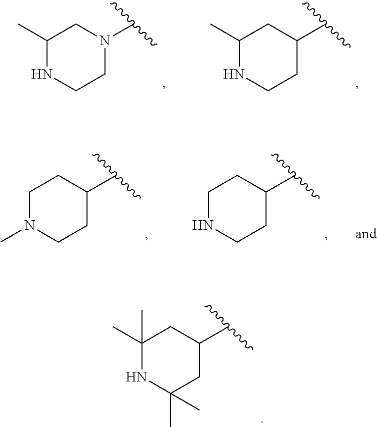

150. The method of any one of the preceding claims, wherein A is selected from

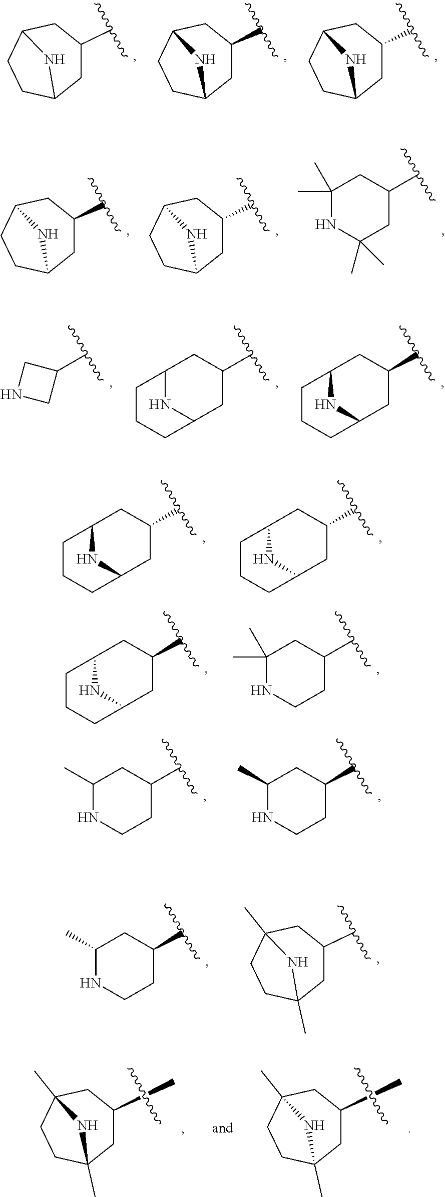

151. The method of any one of the preceding claims, wherein B is selected from

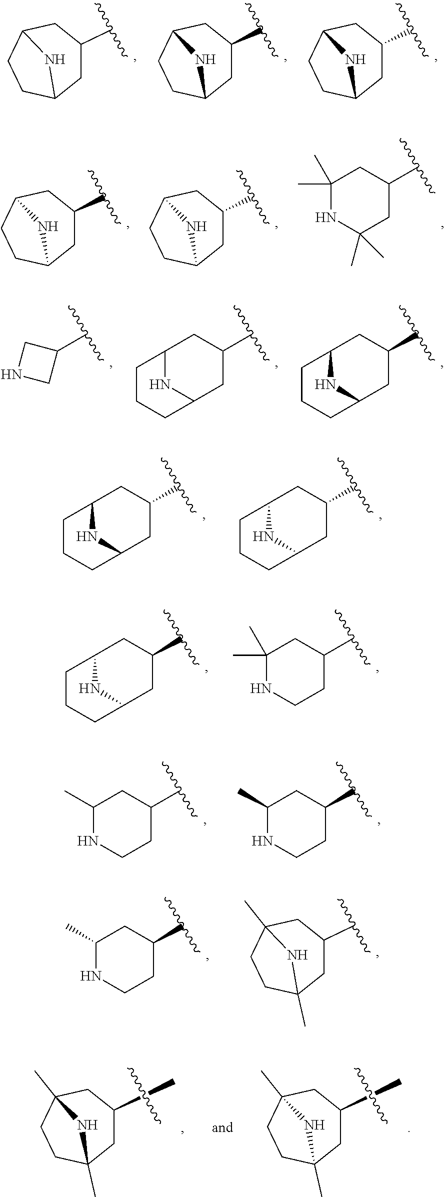

152. The method of any one of the preceding claims, wherein each of A and B is independently heteroaryl or heterocyclyl, each of which is optionally substituted with one or more R¹.

153. The method of any one of the preceding claims, wherein one of A and B is independently a monocyclic heteroaryl or bicyclic heteroaryl, each of which is optionally substituted with one or more R¹.

154. The method of any one of the preceding claims, wherein one of A and B is independently a bicyclic heteroaryl optionally substituted with one or more R¹.

155. The method of any one of the preceding claims, wherein one of A and B is independently a nitrogen-containing heteroaryl optionally substituted with one or more R¹.

156. The method of any one of the preceding claims, wherein one of A and B is a 5-10 membered heteroaryl optionally substituted with one or more R¹.

157. The method of any one of the preceding claims, wherein one of A and B is independently selected from RN

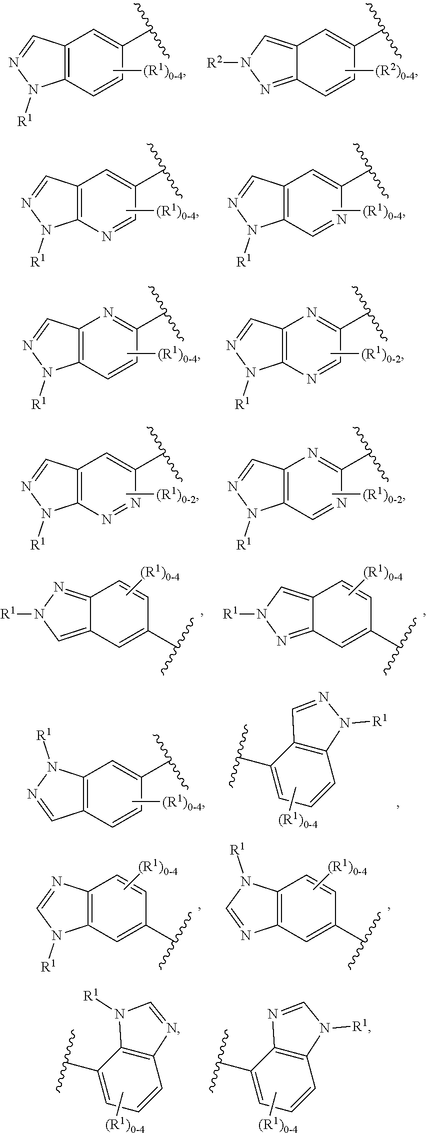

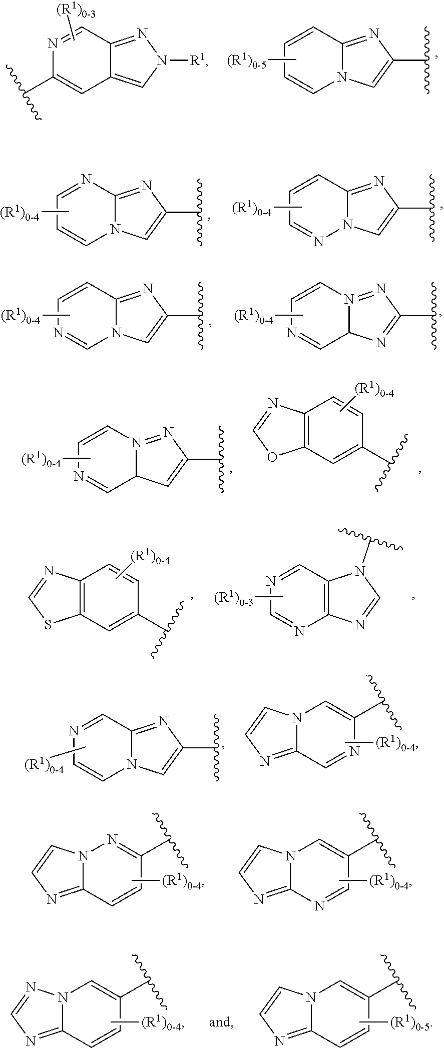

158. The method of any one of the preceding claims, wherein one of A and B is independently selected from 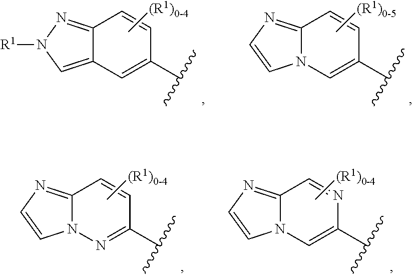
wherein R¹ is as described in claim 137.

159. The method of any one of the preceding claims, wherein one of A and B is independently 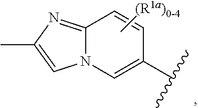
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

160. The method of any one of the preceding claims, wherein A is independently selected from 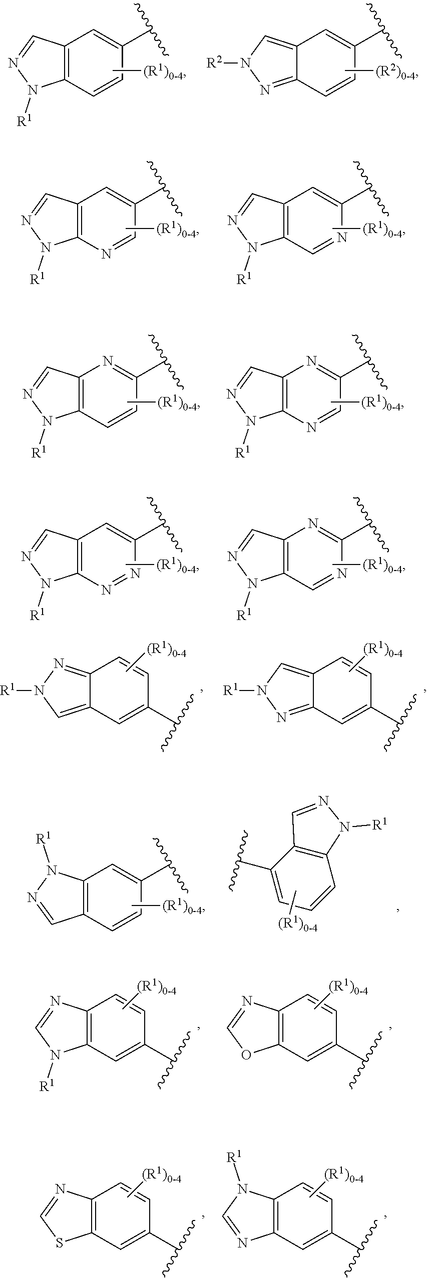 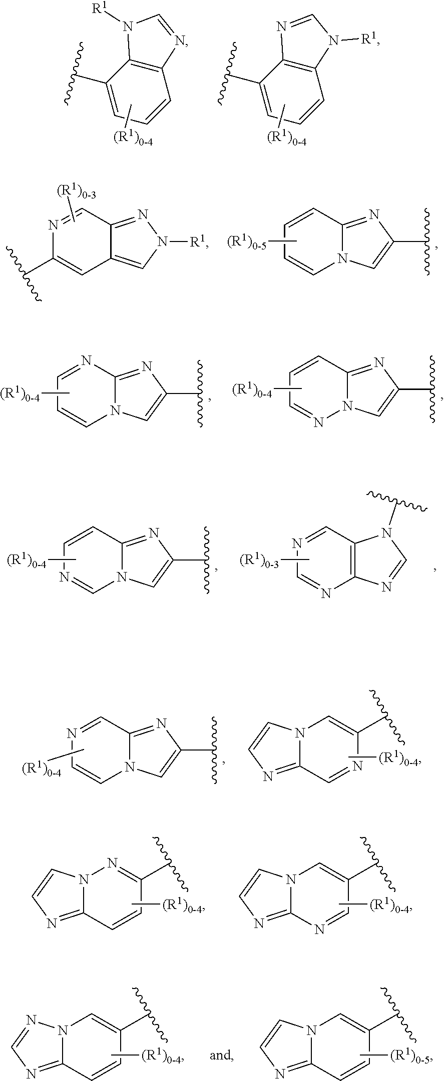
and R¹ is as described in claim 137.

161. The method of any one of the preceding claims, wherein B is independently selected from 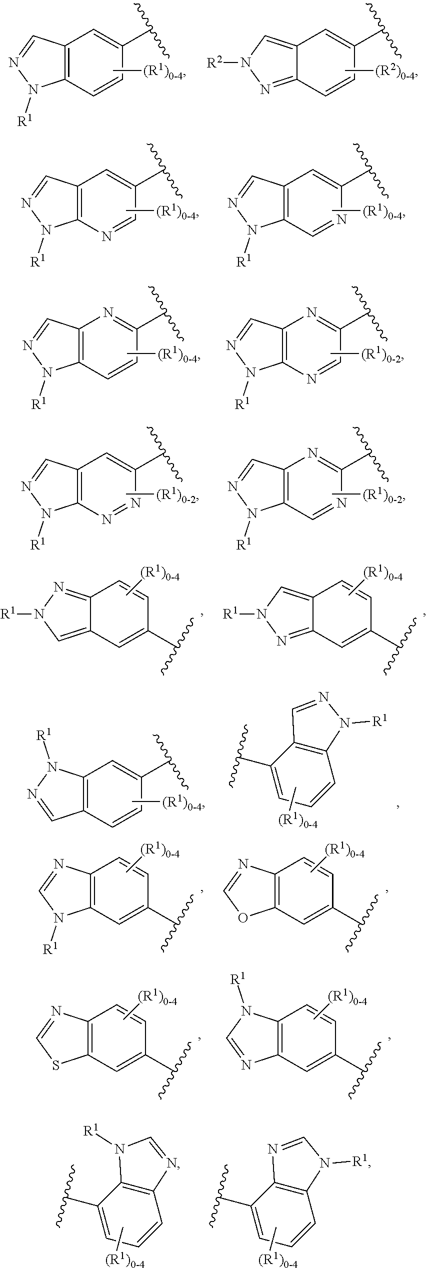 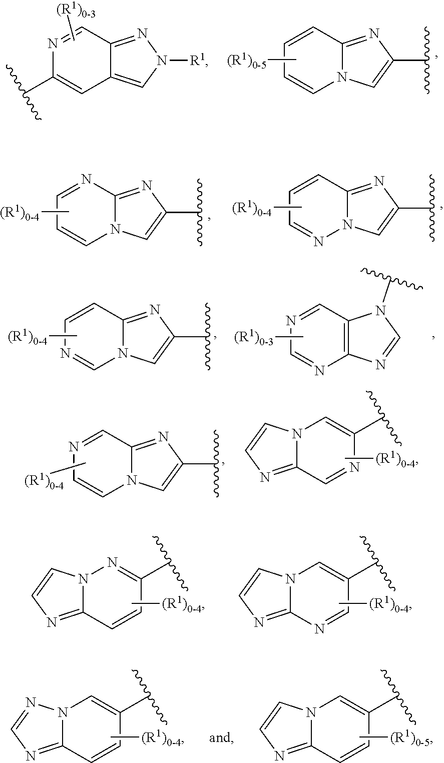
and R¹ is as described in claim 137.

162. The method of any one of the preceding claims, wherein one of A and B is independently is selected from

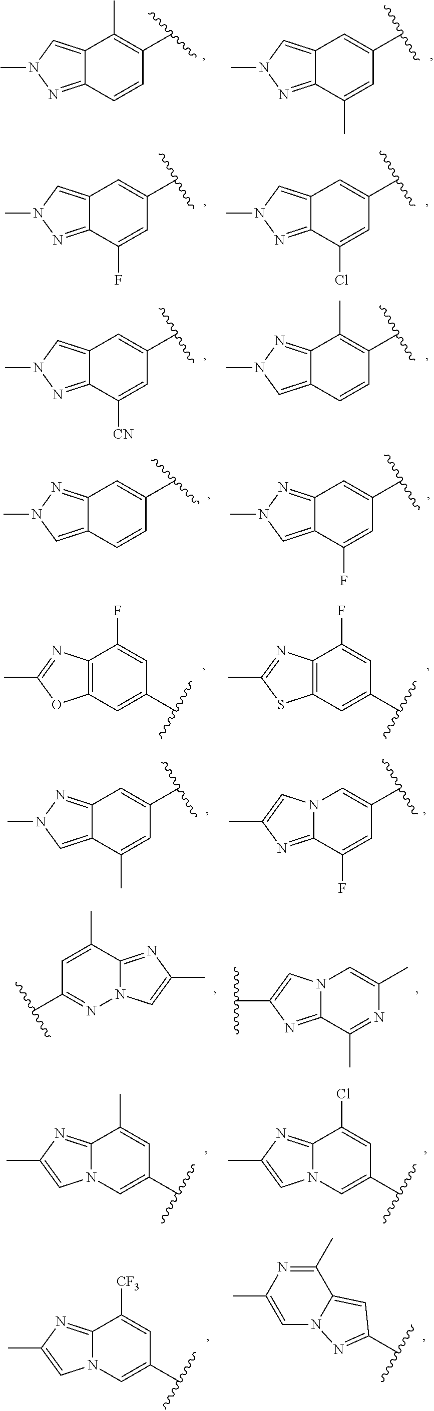

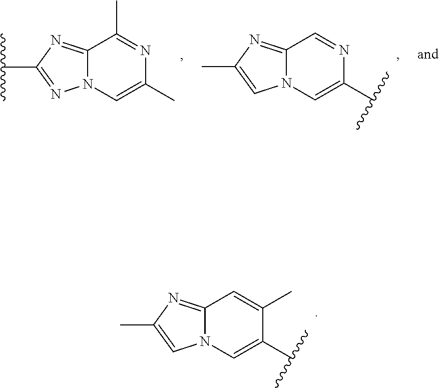

163. The method of any one of the preceding claims, wherein A is independently is selected from

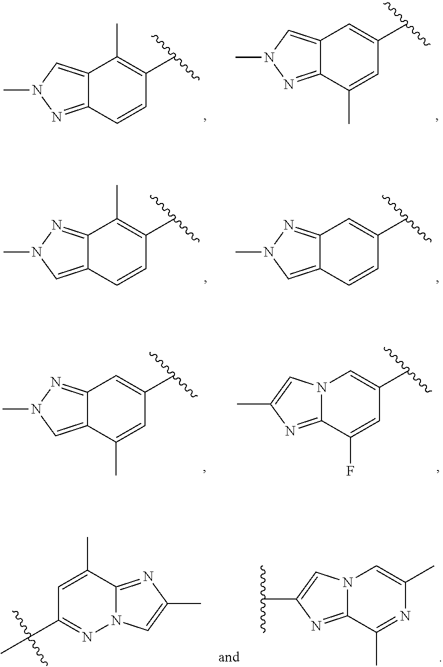

164. The method of any one of the preceding claims, wherein B is independently is selected from

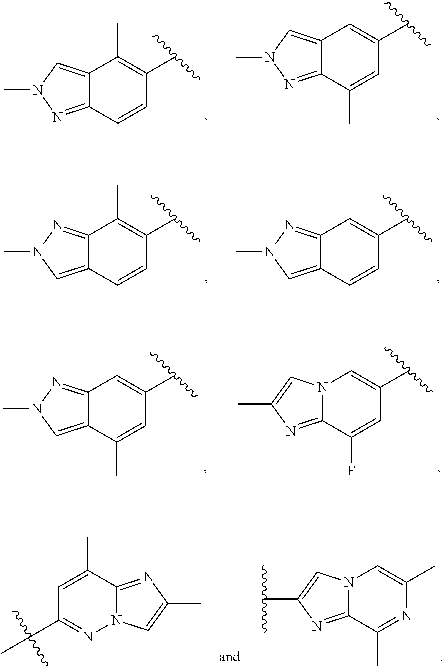

165. The method of any one of the preceding claims, wherein L² is absent, C₁-C₆-alkylene, C₁-C₆-heteroalkylene, —N(R³)C(O)—, or —C(O)N(R³)—, wherein each alkylene and heteroalkylene is optionally substituted with one or more R⁴.

166. The method of any one of the preceding claims, wherein L² is absent or —C(O)N(R³)—.

167. The method of any one of the preceding claims, wherein 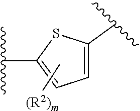
is selected from 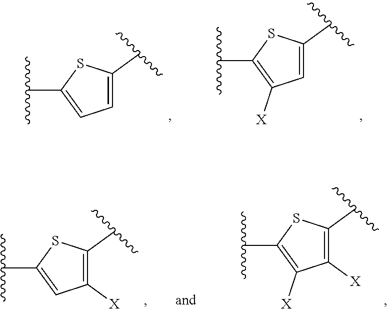
wherein each X is independently halo (e.g., fluoro, chloro, bromo, or iodo).

168. The method of any one of the preceding claims, wherein 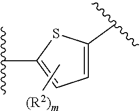
is

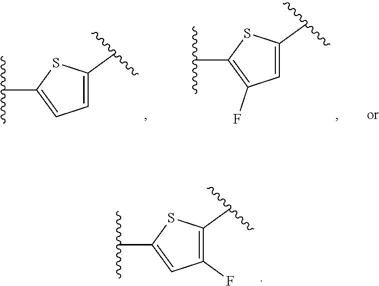 [SMILES: *c1ccc(*)s1 ; *c1cc(F)c(*)s1 ; *c1cc(F)c(*)s1]

169. The method of any one of the preceding claims, wherein m is 1 and R² is halo (e.g., fluoro).

170. The method of any one of the preceding claims, wherein the compound is a compound of Formula (III-a): 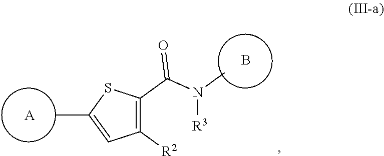
or a pharmaceutically acceptable salt, solvate, hydrate, tautomer, or stereoisomer thereof, wherein: A, R², R³, and B are as described in claim 137.

171. The method of any one of the preceding claims, wherein the compound is a compound of Formula (III-b): 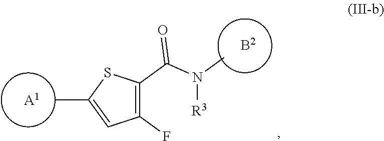
or a pharmaceutically acceptable salt, solvate, hydrate, tautomer, or stereoisomer thereof, wherein R³ is as described in claim 137, A¹ is monocyclic or bicyclic heterocyclyl, each of which is optionally substituted with one or more R¹, and B¹ is monocyclic or bicyclic heteroaryl, each of which is optionally substituted with one or more R¹.

172. The method of any one of the preceding claims, wherein the compound is selected from a compound listed in Table 3 or a pharmaceutically acceptable salt, solvate, hydrate, tautomer, or stereoisomer thereof.

173. The method of any one of the preceding claims, wherein the proliferative disease is selected from a cancer, a benign neoplasm, or angiogenesis.

174. The method of any one of the preceding claims, wherein the proliferative disease is cancer.

175. The method of any one of claims 173-174, wherein the cancer is selected form adenoid cystic carcinoma, colorectal cancer, leukemia, lung cancer, prostate cancer, breast cancer, or ovarian cancer.

176. The method of any one of claims 137-175, wherein the compound is formulated as a pharmaceutical composition further comprising a pharmaceutically acceptable excipient.

177. The method of any one of claims 137-176, wherein the compound alters a target nucleic acid (e.g., an RNA, e.g., a pre-mRNA).

178. The method of any one of claims 137-177, wherein the compound binds to a target nucleic acid (e.g., an RNA, e.g., a pre-mRNA).

179. The method of any one of claims 137-178, wherein the compound stabilizes a target nucleic acid (e.g., an RNA, e.g., a pre-mRNA).

180. The method of any one of claims 137-179, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

181. The method of any one of claims 137-180, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

182. A composition for use in treating a proliferative disease in a subject comprising administering to the subject a compound of Formula (III): 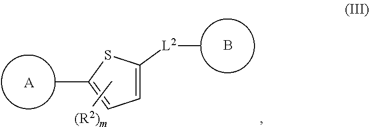
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L² is absent, C₁-C₆-alkylene, C₁-C₆-heteroalkylene, —O—, —C(O)—, —N(R³)—, —N(R³)C(O)—, or —C(O)N(R³)—, wherein alkylene and heteroalkylene are optionally substituted with one or more R⁴;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁵; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁵;
each R² is independently C₁-C₆-alkyl, C₂-C₆-alkenyl, C₂-C₆-alkynyl, C₁-C₆-heteroalkyl, C₁-C₆-haloalkyl, halo, cyano, or —OR^{A};
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁵ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, oxo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R⁷ is independently C₁-C₆-alkyl or halo;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆-heteroalkyl, C₁-C₆ haloalkyl, cycloalkyl, heterocyclyl, —OR^{A}, wherein each alkyl, heteroalkyl, haloalkyl, cycloalkyl, heterocyclyl is optionally substituted with one or more R⁷; or
R^{B} and R^{C} together with the nitrogen atom to which they are attached form a 3-7-membered heterocyclyl or heteroaryl, wherein each heterocyclyl or heteroaryl is optionally substituted with one or more R⁷;
each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
m is 0, 1, or 2; and
x is 0, 1, or 2.

183. The composition for use of claim 182, wherein the compound is formulated as a pharmaceutical composition.

184. The composition for use of any one of claims 182-183, wherein the proliferative disease is cancer.

185. The composition for use of any one of claims 182-184f, wherein the cancer is selected form adenoid cystic carcinoma, colorectal cancer, leukemia, lung cancer, prostate cancer, breast cancer, or ovarian cancer.
